(19) United States
(12) Patent Application Publication
(10) Pub. No.: US 2026/0116868 A1
(43) Pub. Date: Apr. 30, 2026

(54) CBL-B INHIBITORS AND METHODS OF USE THEREOF

(71) Applicant: Arcus Biosciences, Inc., Hayward, CA (US)

(72) Inventors: Srikanth Kumar Gangam, Fremont, CA (US); Clayton Hardman, San Francisco, CA (US); Kenneth Victor Lawson, San Francisco, CA (US); Manmohan Reddy Leleti, Dublin, CA (US); Dongdong Liu, Fremont, CA (US); Artur Karenovich Mailyan, Hayward, CA (US); Masa Podunavac, Hayward, CA (US); Shiwei Qu, Alameda, CA (US); Xianglin Yin, Hayward, CA (US); Kai Yu, Hayward, CA (US); Tzu-Yu Yu, Hayward, CA (US)

(73) Assignee: Arcus Biosciences, Inc., Hayward, CA (US)

(21) Appl. No.: 18/906,525

(22) Filed: Oct. 4, 2024

Related U.S. Application Data
(60) Provisional application No. 63/542,921, filed on Oct. 6, 2023.
(60) Provisional application No. 63/555,807, filed on Feb. 20, 2024.
(60) Provisional application No. 63/641,373, filed on May 1, 2024.

(51) Int. Cl. C07D 401/14; A61K 31/4427; A61K 31/444; A61K 31/4545; A61K 31/4995; A61K 31/5377; C07D 213/82; C07D 401/12; C07D 405/14; C07D 413/14; C07D 491/107

(52) U.S. Cl. CPC C07D 401/14; A61K 31/4427; A61K 31/444; A61K 31/4545; A61K 31/4995; A61K 31/5377; C07D 213/82; C07D 401/12; C07D 405/14; C07D 413/14; C07D 491/107

(57) ABSTRACT

Disclosed herein are compounds that are Cbl-b inhibitors having a structure according to Formula I, and compositions containing those compounds. Methods of preparing the compounds, and methods of using the compounds for the treatment of diseases, disorders, or conditions are also described.

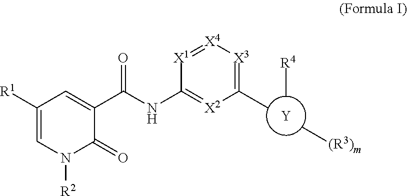

### CROSS-REFERENCE TO RELATED APPLICATIONS

This application claims the benefit of priority to U.S. Provisional Patent Application No. 63/542,921, filed on Oct. 6, 2023, U.S. Provisional Patent Application No. 63/555,807, filed on Feb. 20, 2024, and U.S. Provisional Patent Application No. 63/641,373, filed on May 1, 2024, the entire contents of each of which is incorporated by reference herein.

### BACKGROUND

The following discussion is provided to aid the reader in understanding the disclosure and is not admitted to describe or constitute prior art thereto.

Crucial to the proper regulation of immune responses is a balance between immune activating and inhibitory signals. One cellular mechanism that regulates diverse aspects of the immune system is ubiquitination. Ubiquitination involves covalent conjugation of monoubiquitin or polyubiquitin chains onto amino acid residues of target proteins. Protein ubiquitination can alter the activity and/or stability of a molecule, and in some instances can also alter localization of the molecule into different cellular compartments. The ubiquitination process is catalyzed by sequential actions of ubiquitin-activating (E1), ubiquitin-conjugating (E2) and ubiquitin-ligating (E3) enzymes. The process of protein ubiquitination is counteracted by deubiquitinases (DUBs), a large family of proteases that cleaves ubiquitin chains. Mammalian cells express more than 600 E3 ligases and about 100 DUBs, which display substrate specificities and regulate specific cellular functions.

An increasing number of E3 ligases and DUBs have been identified as important regulators of immune responses. For example, small-molecule inhibitors that are antagonists of the IAP family of E3 ligases, including cIAP1, cIAP2, and X-linked IAP (XIAP), have been developed as small-molecule mimetics of the endogenous IAP inhibitor Smac. Small molecule inhibitors have also been developed against MDM2, an E3 ligase that promotes tumor growth and progression by mediating ubiquitin-dependent degradation of the tumor suppressor p53 and p53-independent functions. Casitas B-lineage lymphoma (Cbl) proteins, a family of E3 ubiquitin ligases, have been previously identified as potential targets; and so has VHL E3 complex, which mediates ubiquitin-dependent degradation of HIF1α and controls metabolic activities and effector function of T cells. Small molecule inhibitors for several DUBs have also been developed, and some of them have been shown to inhibit tumor growth in animal models.

While these potential targets provide exciting opportunities for the immunotherapy field, there remains a need in the art for effective inhibitors.

### SUMMARY

Casitas B-lineage lymphoma (Cbl) proteins are a family of E3 ubiquitin ligases. The mammalian Cbl family contains three homologs—c-Cbl, Cbl-b, and Cbl-3. Cbl-b and c-Cbl share some structural similarities but may have distinct physiological functions.

A compound having a structure according to Formula I:

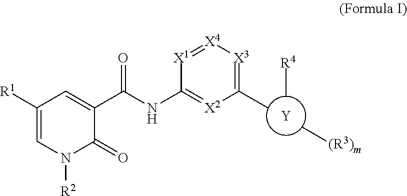

or a pharmaceutically acceptable salt thereof, wherein:
R¹ is selected from the group consisting of —H, —C₁-C₆ haloalkyl, —C₁-C₆ hydroxyalkyl, —C(O)NH₂, —C(O)—(C₁-C₆-alkyl), -(Q¹)-NR^{1a}R^{1b}, -(Q¹)-(C₃-C₇ cycloalkyl), -(Q¹)-phenyl, -(Q¹)-(5- to 6-membered heteroaryl), and -(Q¹)-(4- to 8-membered heterocycloalkyl); wherein said 5- to 6-membered heteroaryl has 1-3 ring heteroatoms independently selected from N, O, and S; said 4- to 8-membered heterocycloalkyl has 1-3 ring heteroatom or heteroatom groups independently selected from N, O, S, and S(O)₂; and said C₃-C₇ cycloalkyl, phenyl, 4- to 8-membered heterocycloalkyl, and 5- to 6-membered heteroaryl are unsubstituted or substituted with 1-2 substituents independently selected from halo, —OH, —C₁-C₃ alkyl, —C₁-C₃ alkoxy, and NH₂;
Q¹ is absent, unsubstituted —(C₁-C₃ alkylene)-, or —(C₁-C₃ alkylene)- substituted with 1-3 R^{q};
each R^{q} is independently halo, —OH, or —NH₂;
R^{1a} and R^{1b} are independently selected from the group consisting of —H, —C₁-C₆ alkyl, —C₁-C₆ haloalkyl, phenyl, —(C₁-C₆ alkylene)-O—(C₁-C₃ alkyl), —C₃-C₆ cycloalkyl, —(C₁-C₃ alkylene)-(C₃-C₆ cycloalkyl), —(C₁-C₃ alkylene)-O—(C₃-C₆ cycloalkyl), —S(O)₂(C₁-C₆ alkyl), 5- to 6-membered heteroaryl having 1-3 ring heteroatoms independently selected from N, O, and S, 4- to 8-membered heterocycloalkyl having 1-3 ring heteroatom or heteroatom groups independently selected from N, O, S, and S(O)₂, and —(C₁-C₃ alkylene)-(4- to 8-membered heterocycloalkyl) wherein said 4- to 8-membered heterocycloalkyl has 1-3 ring heteroatom or heteroatom groups independently selected from N, O, S, and S(O)₂; wherein said phenyl, —(C₁-C₆ alkylene)-O—(C₁-C₃ alkyl), —C₃-C₆ cycloalkyl, —(C₁-C₃ alkylene)-(C₃-C₆ cycloalkyl), 5- to 6-membered heteroaryl, 4- to 8-membered heterocycloalkyl, and —(C₁-C₃ alkylene)-(4- to 8-membered heterocycloalkyl) are unsubstituted or substituted with 1-3 R^{1c};
each R^{1c}, when present, is independently halo, —OH, —C₁-C₃ alkyl, —C₁-C₃ hydroxyalkyl, —C₁-C₃ haloalkyl, —C₁-C₃ alkoxy, or —(C₁-C₃ alkylene)-O—(C₁-C₃ alkyl);
R² is —H, —C₁-C₆ alkyl, —C₂-C₆ alkenyl, —C₁-C₆ haloalkyl, —C₁-C₆ hydroxyalkyl, —C₃-C₈ cycloalkyl, —(C₁-C₃ alkylene)-(C₃-C₈-cycloalkyl), and —(C₁-C₃ alkylene)-(3- to 8-membered heterocycloalkyl), wherein said 3- to 8-membered heterocycloalkyl has 1-3 ring heteroatoms independently selected from N, O, and S;
X¹, X² and X³ are each independently N or CH;
X⁴ is N or CR^{X};
R^{X}, when present, is —H, halo, —CN, —OH, —C₁-C₆ alkyl, —C₁-C₆ haloalkyl, —C₁-C₆ alkoxy, —C₁-C₆ haloalkoxy, —NR^{Xa}R^{Xb}, or —C₃-C₈ cycloalkyl;
R^{Xa} and R^{Xb} are independently —H, —C₁-C₃ alkyl, or —(C₁-C₃ alkylene)-NR^{Xc}R^{Xd};
R^{Xc} and R^{Xd} are independently —H, or —C₁-C₃ alkyl;
ring Y is —C₃-C₆ cycloalkylene, 3- to 6-membered heterocycloalkylene having 1-2 ring heteroatoms independently selected from N, O, and S, phenylene, or 5- to 6-membered heteroarylene having 1-2 ring heteroatoms independently selected from N, O, and S;
m is 0, 1, 2, or 3;
each R³ when present, is independently halo, —CN, —C₁-C₆ alkyl, —C₁-C₆ haloalkyl, —C(O)OR^{3a}, —C(O)NR^{3b}R^{3c}, or —C₁-C₆ alkoxy, or —C₁-C₆ haloalkoxy;
R^{3a} is H or C₁-C₃ alkyl;
R^{3b} and R^{3c} are each independently —H or C₁-C₃ alkyl;
R⁴ is —C(O)NR^{4a}R^{4b}, or a 5- to 6-membered heteroaryl having 1 to 4 ring heteroatoms independently selected from N, O, and S, wherein said 5- to 6-membered heteroaryl is unsubstituted or substituted with 1-3 R^{4c};
R^{4a} and R^{4b} are each independently H, —C₁-C₆ alkyl, —C₁-C₆ haloalkyl, phenyl, or —(C₁-C₃ alkylene)-O—(C₁-C₃ alkyl); or
R^{4a} and R^{4b} taken together with the N atom to which they are attached form a 4- to 8-membered heterocycloalkyl having 0-1 additional ring heteroatom selected from N, O, and S; wherein said 4- to 8-membered heterocycloalkyl is unsubstituted or substituted with 1-3 substituents independently selected from the group consisting of halo, —CN, —C₁-C₆ alkyl, and —C₁-C₆ alkoxy; and
each R^{4c} is independently selected from the group consisting of —CN, —C₁-C₃ alkyl, —C₁-C₃ haloalkyl, and —C₁-C₃ hydroxyalkyl.

In another aspect, this disclosure is directed to methods of inhibiting Cbl-b in a subject comprising administering to the subject an effective amount of a compound described herein.

In another aspect, this disclosure is directed to methods of increasing immune cell activity in a subject comprising administering to the subject an effective amount of a compound described herein.

In yet another aspect, this disclosure provides methods for treating a disease, disorder, or condition mediated at least in part by Cbl-b in a subject, comprising administering to the subject a therapeutically effective amount of a compound described herein. Diseases, disorders, and conditions mediated by Cbl-b include cancer and cancer-related disorders.

Certain aspects of the present disclosure further comprise the administration of one or more additional therapeutic agents as set forth herein below.

### DETAILED DESCRIPTION OF THE DISCLOSURE

Before the present disclosure is further described, it is to be understood that the disclosure is not limited to the particular embodiments set forth herein, and it is also to be understood that the terminology used herein is for the purpose of describing particular embodiments only and is not intended to be limiting.

### Definitions

Unless otherwise defined, all terms of art, notations and other scientific terms or terminology used herein are intended to have the meanings commonly understood by those of skill in the art to which this disclosure pertains.

The term “about” as used herein has its original meaning of approximately and is to provide literal support for the exact number that it precedes, as well as a number that is near to or approximately the number that the term precedes. In general, the term “about” refers to the usual error range for the respective value readily known to the skilled person in this technical field. If the degree of approximation is not otherwise clear from the context, “about” means either within plus or minus 10% of the provided value, or rounded to the nearest significant FIGURE, in all cases inclusive of the provided value. Where ranges are provided, they are inclusive of the boundary values.

The term “alkyl”, by itself or as part of another substituent, means, unless otherwise stated, a saturated monovalent hydrocarbon radical, having, in some embodiments, one to eight (e.g., C₁-C₈ alkyl), or one to six (e.g., C₁-C₆ alkyl), or one to three (e.g., C₁-C₃ alkyl) carbon atoms, respectively. The term “alkyl” encompasses straight and branched-chain hydrocarbon groups. Examples of alkyl groups include, but are not limited to, methyl (Me), ethyl (Et), n-propyl, isopropyl, n-butyl, t-butyl, isobutyl, sec-butyl, isopentyl, tert-pentyl, n-pentyl, isohexyl, n-hexyl, n-heptyl, 4-isopropylheptane, n-octyl, and the like. In some embodiments, the alkyl groups are C₁-C₄ alkyl groups (e.g., methyl, ethyl, isopropyl, isobutyl, or t-butyl). In some embodiments, the alkyl groups are C₁-C₃ alkyl groups (e.g., methyl, ethyl, n-propyl, or isopropyl).

The term “alkenyl”, as used herein, refers to a straight or branched monovalent hydrocarbon radical having, in some embodiments, two to eight carbon atoms (e.g., C₂-C₈ alkenyl), or two to six carbon atoms (e.g., C₂-C₆ alkenyl), or two to three carbon atoms (e.g., C₂-C₃ alkenyl), and having at least one carbon-carbon double bond. Examples of alkenyl groups include, but are not limited to, ethenyl, propenyl, isobutenyl, butadienyl and the like.

The term “alkylene” refers to a straight or branched, saturated, hydrocarbon radical having, in some embodiments, one to six (e.g., C₁-C₆ alkylene), one to four (e.g., C₁-C₄ alkylene), one to three (e.g., C₁-C₃ alkylene), or one to two (e.g., C₁-C₂ alkylene) carbon atoms, and linking at least two other groups, i.e., a divalent hydrocarbon radical. The two moieties linked to the alkylene can be attached to the same carbon atom (i.e., geminal), or different carbon atoms of the alkylene group. For instance, a straight chain alkylene can be the bivalent radical of —(CH₂)ₙ—, where n is 1, 2, 3, 4, 5 or 6 (i.e., a C₁-C₆ alkylene). Representative alkylene groups include, but are not limited to, methylene, ethylene, propylene, isopropylene, butylene, isobutylene, secbutylene, pentylene, hexylene and the like. In some embodiments, the alkylene groups are C₁-C₂ alkylene groups (e.g., methylene, or ethylene). In some embodiments, the alkylene groups are C₁-C₄ alkylene groups (e.g., methylene, ethylene, propylene, isopropylene, butylene, isobutylene, sec-butylene, and the like).

As used herein, the term “alkoxy” refers to an alkyl group, as defined herein, that is attached to the remainder of the molecule via an oxygen atom (e.g., —O—C₁-C₁₂ alkyl, —O—C₁-C₈ alkyl, —O—C₁-C₆ alkyl, or —O—C₁-C₃ alkyl). Non-limiting examples of alkoxy groups include methoxy, ethoxy, n-propoxy, iso-propoxy, n-butoxy, sec-butoxy, n-pentoxy, n-hexoxy, and the like. In some embodiments, the alkoxy groups are C₁-C₃ alkoxy groups (e.g., methoxy, ethoxy, n-propoxy, or iso-propoxy).

The term “cycloalkyl” refers to a monocyclic, bicyclic or polycyclic hydrocarbon ring system having, in some embodiments, 3 to 14 carbon atoms (e.g., C₃-C₁₄ cycloalkyl), or 3 to 10 carbon atoms (e.g., C₃-C₁₀ cycloalkyl), or 3 to 8 carbon atoms (e.g., C₃-C₈ cycloalkyl), or 3 to 6 carbon atoms (e.g., C₃-C₆ cycloalkyl), or 3 to 4 carbon atoms (e.g., C₃-C₄ cycloalkyl). Cycloalkyl groups can be saturated or characterized by one or more points of unsaturation (i.e., carbon-carbon double and/or triple bonds), provided that the points of unsaturation do not result in an aromatic system. Examples of monocyclic cycloalkyl groups include, but are not limited to, cyclopropyl, cyclobutyl, cyclopentyl, cyclohexyl, cyclohexenyl, cycloheptyl, cycloheptenyl, cycloheptadienyl, cyclooctyl, cyclooctenyl, cyclooctadienyl, and the like. The rings of bicyclic and polycyclic cycloalkyl groups can be fused, bridged, or spirocyclic. Non-limiting examples of bicyclic, spirocyclic and polycyclic cycloalkyl groups include bicyclo[2.2.1]heptane, bicyclo[2.2.2]octane, adamantyl, indanyl, spiro[5.5]undecane, spiro[2.2]pentane, spiro[2.2]pentadiene, spiro[2.3]hexane, spiro[2.5]octane, spiro[2.2]pentadiene, and the like. In some embodiments, the cycloalkyl groups of the present disclosure are monocyclic C₃-C₆ cycloalkyl moieties (e.g., cyclopropyl, cyclobutyl, cyclopentyl, or cyclohexyl). In some embodiments, the cycloalkyl groups of the present disclosure are monocyclic C₃-C₄ cycloalkyl moieties (e.g., cyclopropyl, or cyclobutyl).

As used herein, the term “cycloalkoxy” refers to an cycloalkyl group, as defined herein, that is attached to the remainder of the molecule via an oxygen atom (e.g., —O—C₃-C₈ cycloalkyl, —O—C₃-C₆ cycloalkyl, —O—C₃-C₅ cycloalkyl, or —O—C₃-C₄ cycloalkyl). Non-limiting examples of cycloalkoxy groups include cyclopropoxy, cyclobutoxy, cyclopentoxy, cyclohexoxy, and the like. In some embodiments, the alkoxy group is cyclopropoxy).

The term “cycloalkylene” refers to a cycloalkyl group as defined herein that links at least two other moieties, i.e., a divalent cycloalkyl group. The two moieties linked to the cycloalkylene group can be attached to the same carbon atom, or different carbon atoms of the cycloalkylene group. Examples of cycloalkyl groups include, but are not limited to, cyclopropylene, cyclobutylene, cyclopentylene, cyclohexylene, cyclohexenylene, and the like. In some embodiments, the cycloalkylene groups are C₃-C₄ cycloalkylene moieties (e.g., cyclopropylene, or cyclobutylene).

The term “heterocycloalkyl” refers to a non-aromatic monocyclic, bicyclic or polycyclic cycloalkyl ring having, in some embodiments, 3 to 14 members (e.g., 3- to 14-membered heterocycle), or 3 to 10 members (e.g., 3- to 10-membered heterocycle), or 3 to 8 members (e.g., 3- to 8-membered heterocycle), or 3 to 6 members (e.g., 3- to 6-membered heterocycle), or 5 to 6 members (e.g., 5- to 6-membered heterocycle), and having from one to five, one to four, one to three, one to two, or one heteroatom or heteroatom groups independently selected from nitrogen (N), oxygen (O), sulfur (S), sulfoxide (S(O)), and sulfone (S(O)₂). Heterocycloalkyl groups are saturated or characterized by one or more points of unsaturation (e.g., one or more carbon-carbon double bonds, carbon-carbon triple bonds, carbon-nitrogen double bonds, and/or nitrogen-nitrogen double bonds), provided that the points of unsaturation do not result in an aromatic system. The rings of bicyclic and polycyclic heterocycloalkyl groups can be fused, bridged, or spirocyclic. Non-limiting examples of heterocycloalkyl groups include aziridine, oxirane, thiirane, pyrrolidine, imidazolidine, pyrazolidine, dioxolane, phthalimide, piperidine, 1,4-dioxane, morpholine, thiomorpholine, thiomorpholine-S-oxide, thiomorpholine-S,S-oxide, piperazine, 3,4,5,6-tetrahydropyridazine, tetrahydropyran, pyran, decahydroisoquinoline, 3-pyrroline, thiopyran, tetrahydrofuran, tetrahydrothiophene, tetrahydro-1,1-dioxido-2H-thiopyran, quinuclidine, 1,4-oxazepane, 2-azabicyclo[4.1.0]heptane, 2-oxa-5-azabicyclo[2.2.1]heptane, 2-azabicyclo[2.2.1]heptane, 2,5-diazabicyclo[2.2.1]heptane, 6-oxa-3-azabicyclo[3.1.1]heptane, 3-oxa-6-azabicyclo[3.1.1]heptane, 2,5-diazabicyclo[2.2.1]heptane, 2-thia-6-azaspiro[3.3]heptane 2,2-dioxide, 2,6-diazaspiro[3.3]heptane, 2-azaspiro[3.3]heptane, 2-oxa-6-azaspiro[3.3]heptane, 1-oxaspiro[3.3]heptane, 5-azaspiro[2.4]heptane, 6-azaspiro[3.4]octane, 6-azaspiro[2.5]octane, 4-oxa-7-azaspiro[2.5]octane, 3-oxa-8-azabicyclo[3.2.1]octane, and the like. A heterocycloalkyl group can be attached to the remainder of the molecule through a ring carbon atom, or a ring heteroatom, when chemically permissible. In some embodiments, the heterocycloalkyl groups of the present disclosure are monocyclic 4- to 8-membered heterocycloalkyl moieties having one or two heteroatom or heteroatom groups independently selected from N and O (e.g., azetidine, piperidine, piperazine, morpholine, pyrrolidine, imidazolidine, pyrazolidine, tetrahydrofuran, tetrahydropyran, 2-oxa-6-azaspiro[3.3]heptane, 5-azaspiro[2.4]heptane, 2-azabicyclo[2.2.1]heptane, 2,5-diazabicyclo[2.2.1]heptane, and the like).

The term “heterocycloalkylene” refers to a heterocycloalkyl group as defined herein that links at least two other moieties, i.e., a divalent heterocycloalkyl group. The two moieties linked to the heterocycloalkylene group can be attached to the same carbon atom, or different atoms (i.e., carbon atoms and/or heteroatoms when chemically permissible) of the heterocycloalkylene group. Examples of heterocycloalkylene groups include, but are not limited to, azetidinylene, oxetanylene, tetrahydrofuranylene, pyrrolidinylene, piperidinylene, piperazinylene, and the like. In some embodiments, the heterocycloalkylene groups of the present disclosure are 4-membered heterocycloalkylene moieties having 1 ring heteroatom selected from N and O (e.g., oxetanylene).

The term “aryl” refers to an aromatic ring system containing one ring, or two, or three rings fused together, and having, in some embodiments, six to fourteen (i.e., C₆-C₁₄ aryl), or six to ten (i.e., C₆-C₁₀ aryl), or six (i.e., C₆ aryl) carbon atoms. Non-limiting examples of aryl groups include phenyl, naphthyl and anthracenyl. In some embodiments, aryl groups are phenyl.

The term “phenylene” refers to a phenyl group that links at least two other moieties, i.e., a divalent phenyl group. The two moieties linked to the phenylene group are attached to different carbon atoms of the phenylene group.

The term “heteroaryl” refers to monocyclic or fused bicyclic aromatic groups (or rings) having, in some embodiments, from 5 to 14 (i.e., 5- to 14-membered heteroaryl), or from 5 to 10 (i.e., 5- to 10-membered heteroaryl), or from 5 to 6 (i.e., 5- to 6-membered heteroaryl) members (i.e., ring vertices), and containing from one to five, one to four, one to three, one to two, or one heteroatom independently selected from nitrogen (N), oxygen (O), and sulfur (S). A heteroaryl group can be attached to the remainder of the molecule through a carbon atom or a heteroatom of the heteroaryl group, when chemically permissible. Non-limiting examples of heteroaryl groups include pyridyl, pyridazinyl, pyrazinyl, pyrimidinyl, triazinyl, purinyl, thienopyridinyl, thienopyrimidinyl, pyrazolopyrimidinyl, imidazopyridines, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like. In some embodiments, the heteroaryl groups of the present disclosure are monocyclic 5- to 6-membered heteroaryl moieties having 1-3 heteroatoms independently selected from N, O, and S (e.g., pyridinyl, pyrimidinyl, pyridazinyl, triazolyl, imidazolyl, pyrazolyl, oxazolyl, oxadiazolyl, or thiazolyl).

The term “heteroarylene” refers to a heteroaryl group as defined herein that links at least two other moieties, i.e., a divalent heteroaryl group. The two moieties linked to the heteroarylene group are attached to different atoms (i.e., carbon atoms and/or heteroatoms when chemically permissible) of the heteroarylene group. Exemplary heteroarylene groups include, but are not limited to pyrazolylene, oxazolylene, oxadiazolylene, imidazolylene, triazolylene, thiazolylene, pyrrolylene, furanylene, thiophenylene, pyridylene, pyrimidinylene, pyridazinylene, and the like.

As used herein, a wavy line, “ [symbol] ”, that intersects a single, double or triple bond in any chemical structure depicted herein, represents that the point of attachment of the single, double, or triple bond to the remainder of the molecule is through either one of the atoms that make up the single, double or triple bond. Additionally, a bond extending from a substituent to the center of a ring (e.g., a phenyl ring) is meant to indicate attachment of that substituent to the ring at any of the available ring vertices, i.e., such that attachment of the substituent to the ring results in a chemically stable arrangement.

The term “halogen”, by itself or as part of another substituent, means, unless otherwise stated, a fluorine, chlorine, bromine, or iodine atom. Additionally, terms such as “haloalkyl” and “haloalkoxy” refer to alkyl groups and alkoxy groups, respectively, as defined herein, that are substituted with one or more halogen(s) (e.g., 1-3 halogen(s)). For example, the term “C₁-C₄ haloalkyl” is meant to include trifluoromethyl, difluoromethyl, 2,2,2-trifluoroethyl, 4-chlorobutyl, 3-bromopropyl, and the like. Additionally, exemplary C₁-C₃ haloalkoxy groups include, but are not limited to, trifluoromethoxy (—OCF₃), difluoromethoxy (—OCF₂H), trifluoroethoxy (—OCH₂CF₃), difluoroethoxy (—OCH₂CF₂H), 2,2-difluoropropoxy (—OCH₂CF₂CH₃) and the like.

The term “hydroxyalkyl” refers to an alkyl group, as defined herein, that is substituted with one or more hydroxyl groups (e.g., 1-3 hydroxyl groups). Exemplary hydroxyalkyl groups include methanol, ethanol, 1,2-propanediol, 1,2-hexanediol, glycerol, and the like.

As referred to herein, “pharmaceutically acceptable salt” is meant to include salts of the compounds according to this disclosure that are prepared with suitably nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from suitably nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present disclosure may contain both basic and acidic functionalities that allow the compounds to be converted into either base or acid addition salts.

The neutral forms of the compounds may be regenerated by contacting the salt with a base or acid and isolating the parent compound.

This disclosure also contemplates isomers of the compounds described herein (e.g., stereoisomers, geometric isomers, and atropisomers). For example, certain compounds of the present disclosure possess asymmetric carbon atoms (chiral centers); poly-substituted non-aromatic cyclic moieties; or hindered rotation about a single bond; the racemates, diastereomers, enantiomers, geometric isomers (e.g., cis and trans isomers), and atropisomers (e.g., Rₐ, Sₐ, P and M isomers) of which are all intended to be encompassed within the scope of the present disclosure. Stereoisomeric forms may be defined, in terms of absolute stereochemistry, as (R) or (S), and/or depicted uses dashes and/or wedges. When a stereochemical depiction (e.g., using dashes, [symbol] , and/or wedges, [symbol] ) is shown in a chemical structure, or a stereochemical assignment (e.g., using (R) and (S) notation) is made in a chemical name, it is meant to indicate that the depicted isomer is present and substantially free of one or more other isomer(s) (e.g., enantiomers and diastereomers, when present). For poly-substituted cyclic moieties, the relative stereochemistry of the geometric isomers may also be depicted uses dashes and/or wedges. When a geometric depiction (e.g., using dashes, [symbol] , and/or wedges, [symbol] ) is shown in a chemical structure, it is meant to indicate that the depicted isomer is present and substantially free of one or more other isomer(s) (e.g., cis-isomers or trans-isomers). “Substantially free of” other isomer(s) indicates at least an 70/30 ratio of the indicated isomer to the other isomer(s), more preferably 80/20, 90/10, or 95/5 or more. In some embodiments, the indicated isomer will be present in an amount of at least 99%. When the absolute (R or S) or relative (cis or trans) stereochemistry of a stereoisomer and/or geometric isomer is not depicted in a structure using dashes and/or wedges, and is instead denoted with a solid line ( [symbol] ), all possible stereoisomers (e.g., enantiomers, diastereomers, [symbol] racemic mixtures, etc.), and/or all possible geometric isomers (cis-isomers, trans-isomers, or mixtures thereof) are contemplated. In such instances, the compound may be present as a racemic mixture, scalemic mixture, a mixture of diastereomers, or a mixture of geometric isomers.

The compounds of the present disclosure may also contain unnatural proportions of atomic isotopes at one or more of the atoms that constitute such compounds. Unnatural proportions of an isotope may be defined as ranging from the amount found in nature to an amount consisting of 100% of the atom in question. For example, the compounds may incorporate radioactive isotopes, such as for example tritium (³H), iodine-125 (¹²⁵I) or carbon-14 (¹⁴C), or non-radioactive isotopes, such as deuterium (²H) or carbon-13 (¹³C). Such isotopic variations can provide additional utilities to those described elsewhere herein. For instance, isotopic variants of the compounds of the disclosure may find additional utility, including but not limited to, as diagnostic and/or imaging reagents, or as cytotoxic/radiotoxic therapeutic agents. Additionally, isotopic variants of the compounds of the disclosure can have altered pharmacokinetic and pharmacodynamic characteristics which can contribute to enhanced safety, tolerability or efficacy during treatment. In some embodiments, the compounds according to this disclosure are characterized by one or more deuterium atoms.

The terms “treat”, “treating”, treatment” and the like refer to a course of action that eliminates, reduces, suppresses, mitigates, ameliorates, or prevents the worsening of, either temporarily or permanently, a disease, disorder or condition to which the term applies, or at least one of the symptoms associated therewith. Treatment includes alleviation of symptoms, diminishment of extent of disease, inhibiting (e.g., arresting the development or further development of the disease, disorder or condition or clinical symptoms association therewith) an active disease, delaying or slowing of disease progression, improving the quality of life, and/or prolonging survival of a subject as compared to expected survival if not receiving treatment or as compared to a published standard of care therapy for a particular disease.

The term “in need of treatment” as used herein refers to a judgment made by a physician or similar professional that a subject requires or will benefit from treatment. This judgment is made based on a variety of factors that are in the realm of the physician's expertise, which may include a positive diagnosis of a disease, disorder or condition.

The terms “prevent”, “preventing”, “prevention”, “prophylaxis” and the like refer to a course of action initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting progression thereof to a harmful or otherwise undesired state. Prevention also refers to a course of action initiated in a subject after the subject has been treated for a disease, disorder, condition or a symptom associated therewith in order to prevent relapse of that disease, disorder, condition or symptom.

The term “in need of prevention” as used herein refers to a judgment made by a physician or other caregiver that a subject requires or will benefit from preventative care. This judgment is made based on a variety of factors that are in the realm of a physician's or caregiver's expertise.

“Substantially pure” indicates that a component (e.g., a compound according to this disclosure) makes up greater than about 50% of the total content of the composition, and typically greater than about 60% of the total content. More typically, “substantially pure” refers to compositions in which at least 75%, at least 85%, at least 90% or more of the total composition is the component of interest. In some cases, the component of interest will make up greater than about 90%, or greater than about 95% of the total content of the composition.

Compounds that are selective may be particularly useful in the treatment of certain disorders or may offer a reduced likelihood of undesired side effects.

Compounds provided herein may have advantageous pharmacokinetic profiles including, for example, low efflux, hepatocyte stability, clearance, and/or inhibition against CYP.

Compounds provided herein may have favorable pharmacokinetic profiles as assessed in a rat model. In some embodiments, compounds according to this disclosure have low clearance in rat. In some embodiments, compounds according to this disclosure have rat clearance (CL) lower than 3 L/hr/kg, or lower than 2 L/hr/kg, or lower than 1 L/hr/kg. In some embodiments, compounds according to this disclosure have long half-life (t½) in rat. In some embodiments, the compounds have a t½ greater than 1 hr, or greater than 1.5 hr, or greater than 2 hr, or greater than 2.5 hr, or greater than 3 hr, or greater than 3.5 hr, or greater than 4 hr, or greater than 4.5 hr, or greater than 5 hr.

### Compounds of the Disclosure

The present disclosure relates to compounds that inhibit the activity of Cbl-b.

In one aspect, this disclosure is directed to a compound having a structure according to Formula I:

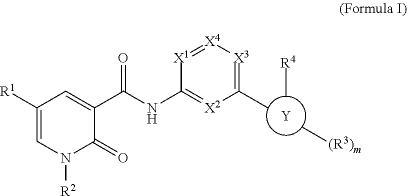

or a pharmaceutically acceptable salt thereof, wherein:
R¹ is selected from the group consisting of —H, —C₁-C₆ haloalkyl, —C₁-C₆ hydroxyalkyl, —C(O)NH₂, —C(O)—(C₁-C₆-alkyl), -(Q¹)-NR^{1a}R^{1b}, -(Q¹)-(C₃-C₇ cycloalkyl), -(Q¹)-phenyl, -(Q¹)-(5- to 6-membered heteroaryl), and -(Q¹)-(4- to 8-membered heterocycloalkyl); wherein said 5- to 6-membered heteroaryl has 1-3 ring heteroatoms independently selected from N, O, and S; said 4- to 8-membered heterocycloalkyl has 1-3 ring heteroatom or heteroatom groups independently selected from N, O, S, and S(O)₂; and said C₃-C₇ cycloalkyl, phenyl, 4- to 8-membered heterocycloalkyl, and 5- to 6-membered heteroaryl are unsubstituted or substituted with 1-2 substituents independently selected from halo, —OH, —C₁-C₃ alkyl, —C₁-C₃ alkoxy, and NH₂;
Q¹ is absent, unsubstituted —(C₁-C₃ alkylene)-, or —(C₁-C₃ alkylene)- substituted with 1-3 R^{q};
each R^{q} is independently halo, —OH, or —NH₂;
R^{1a} and R^{1b} are independently selected from the group consisting of —H, —C₁-C₆ alkyl, —C₁-C₆ haloalkyl, phenyl, —(C₁-C₆ alkylene)-O—(C₁-C₃ alkyl), —C₃-C₆ cycloalkyl, —(C₁-C₃ alkylene)-(C₃-C₆ cycloalkyl), —(C₁-C₃ alkylene)-O—(C₃-C₆ cycloalkyl), —S(O)₂(C₁-C₆ alkyl), 5- to 6-membered heteroaryl having 1-3 ring heteroatoms independently selected from N, O, and S, 4- to 8-membered heterocycloalkyl having 1-3 ring heteroatom or heteroatom groups independently selected from N, O, S, and S(O)₂, and —(C₁-C₃ alkylene)-(4- to 8-membered heterocycloalkyl) wherein said 4- to 8-membered heterocycloalkyl has 1-3 ring heteroatom or heteroatom groups independently selected from N, O, S, and S(O)₂; wherein said phenyl, —(C₁-C₆ alkylene)-O—(C₁-C₃ alkyl), —C₃-C₆ cycloalkyl, —(C₁-C₃ alkylene)-(C₃-C₆ cycloalkyl), 5- to 6-membered heteroaryl, 4- to 8-membered heterocycloalkyl, and —(C₁-C₃ alkylene)-(4- to 8-membered heterocycloalkyl) are unsubstituted or substituted with 1-3 R^{1c};
each R^{1c}, when present, is independently halo, —OH, —C₁-C₃ alkyl, —C₁-C₃ hydroxyalkyl, —C₁-C₃ haloalkyl, —C₁-C₃ alkoxy, or —(C₁-C₃ alkylene)-O—(C₁-C₃ alkyl);
R² is —H, —C₁-C₆ alkyl, —C₂-C₆ alkenyl, —C₁-C₆ haloalkyl, —C₁-C₆ hydroxyalkyl, —C₃-C₈ cycloalkyl, —(C₁-C₃ alkylene)-(C₃-C₈-cycloalkyl), and —(C₁-C₃ alkylene)-(3- to 8-membered heterocycloalkyl), wherein said 3- to 8-membered heterocycloalkyl has 1-3 ring heteroatoms independently selected from N, O, and S;
X¹, X² and X³ are each independently N or CH;
X⁴ is N or CR^{X};
R^{X}, when present, is —H, halo, —CN, —OH, —C₁-C₆ alkyl, —C₁-C₆ haloalkyl, —C₁-C₆ alkoxy, —C₁-C₆ haloalkoxy, —NR^{Xa}R^{Xb}, or —C₃-C₈ cycloalkyl;
R^{Xa} and R^{Xb} are independently —H, —C₁-C₃ alkyl, or —(C₁-C₃ alkylene)-NR^{Xc}R^{Xd};
R^{Xc} and R^{Xd} are independently —H, or —C₁-C₃ alkyl;
ring Y is —C₃-C₆ cycloalkylene, 3- to 6-membered heterocycloalkylene having 1-2 ring heteroatoms independently selected from N, O, and S, phenylene, or 5- to 6-membered heteroarylene having 1-2 ring heteroatoms independently selected from N, O, and S;
m is 0, 1, 2, or 3;
each R³ when present, is independently halo, —CN, —C₁-C₆ alkyl, —C₁-C₆ haloalkyl, —C(O)OR^{3a}, —C(O)NR^{3b}R^{3c}, —C₁-C₆ alkoxy, or —C₁-C₆ haloalkoxy;
R^{3a} is H or C₁-C₃ alkyl;
R^{3b} and R^{3c} are each independently —H or C₁-C₃ alkyl;
R⁴ is —C(O)NR^{4a}R^{4b}, or a 5- to 6-membered heteroaryl having 1 to 4 ring heteroatoms independently selected from N, O, and S, wherein said 5- to 6-membered heteroaryl is unsubstituted or substituted with 1-3 R^{4c};
R^{4a} and R^{4b} are each independently H, —C₁-C₆ alkyl, —C₁-C₆ haloalkyl, phenyl, or —(C₁-C₃ alkylene)-O—(C₁-C₃ alkyl); or
R^{4a} and R^{4b} taken together with the N atom to which they are attached form a 4- to 8-membered heterocycloalkyl having 0-1 additional ring heteroatom selected from N, O, and S; wherein said 4- to 8-membered heterocycloalkyl is unsubstituted or substituted with 1-3 substituents independently selected from the group consisting of halo, —CN, —C₁-C₆ alkyl, and —C₁-C₆ alkoxy; and
each R^{4c} is independently selected from the group consisting of —CN, —C₁-C₃ alkyl, —C₁-C₃ haloalkyl, and —C₁-C₃ hydroxyalkyl.

In some embodiments, the compound has a structure according to Formula I:

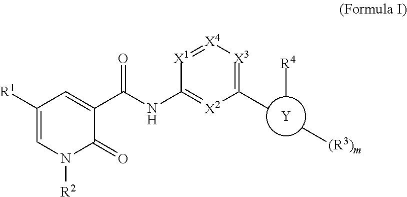

or a pharmaceutically acceptable salt thereof, wherein:
R¹ is selected from the group consisting of —H, —C₁-C₆ haloalkyl, —C₁-C₆ hydroxyalkyl, —C(O)NH₂, —C(O)—(C₁-C₆-alkyl), -(Q¹)-NR^{1a}R^{1b}, -(Q¹)-(C₃-C₇ cycloalkyl), -(Q¹)-phenyl, -(Q¹)-(5- to 6-membered heteroaryl), and -(Q¹)-(4- to 8-membered heterocycloalkyl); wherein said 5- to 6-membered heteroaryl has 1-3 ring heteroatoms independently selected from N, O, and S; said 4- to 8-membered heterocycloalkyl has 1-3 ring heteroatom or heteroatom groups independently selected from N, O, S, and S(O)₂; and said C₃-C₇ cycloalkyl, phenyl, 4- to 8-membered heterocycloalkyl, and 5- to 6-membered heteroaryl are unsubstituted or substituted with 1-2 substituents independently selected from halo, —OH, —C₁-C₃ alkyl, —C₁-C₃ alkoxy, and NH₂;
Q¹ is absent, unsubstituted —(C₁-C₃ alkylene)-, or —(C₁-C₃ alkylene)- substituted with 1-3 R^{q};
each R^{q} is independently halo, —OH, or —NH₂;
R^{1a} and R^{1b} are independently selected from the group consisting of —H, —C₁-C₆ alkyl, —C₁-C₆ haloalkyl, phenyl, —(C₁-C₆ alkylene)-O—(C₁-C₃ alkyl), —C₃-C₆ cycloalkyl, —(C₁-C₃ alkylene)-(C₃-C₆ cycloalkyl), —(C₁-C₃ alkylene)-O—(C₃-C₆ cycloalkyl), —S(O)₂(C₁-C₆ alkyl), 5- to 6-membered heteroaryl having 1-3 ring heteroatoms independently selected from N, O, and S, and 4- to 8-membered heterocycloalkyl having 1-3 ring heteroatom or heteroatom groups independently selected from N, O, S, and S(O)₂; wherein said phenyl, —(C₁-C₆ alkylene)-O—(C₁-C₃ alkyl), —C₃-C₆ cycloalkyl, —(C₁-C₃ alkylene)-(C₃-C₆ cycloalkyl), 5- to 6-membered heteroaryl, and 4- to 8-membered heterocycloalkyl are unsubstituted or substituted with 1-3 R^{1c};
each R^{1c}, when present, is independently halo, —OH, —C₁-C₃ alkyl, —C₁-C₃ hydroxyalkyl, —C₁-C₃ haloalkyl, —C₁-C₃ alkoxy, or —(C₁-C₃ alkylene)-O—(C₁-C₃ alkyl);
R² is —H, —C₁-C₆ alkyl, —C₂-C₆ alkenyl, —C₁-C₆ haloalkyl, —C₁-C₆ hydroxyalkyl, —C₃-C₈ cycloalkyl, —(C₁-C₃ alkylene)-(C₃-C₈-cycloalkyl), and —(C₁-C₃ alkylene)-(3- to 8-membered heterocycloalkyl), wherein said 3- to 8-membered heterocycloalkyl has 1-3 ring heteroatoms independently selected from N, O, and S;
X¹, X² and X³ are each independently N or CH;
X⁴ is N or CR^{X};
R^{X}, when present, is —H, halo, —CN, —OH, —C₁-C₆ alkyl, —C₁-C₆ haloalkyl, —C₁-C₆ alkoxy, —NR^{Xa}R^{Xb}, or —C₃-C₈ cycloalkyl;
R^{Xa} and R^{Xb} are independently —H, —C₁-C₃ alkyl, or —(C₁-C₃ alkylene)-NR^{Xc}R^{Xd};
R^{Xc} and R^{Xd} are independently —H, or —C₁-C₃ alkyl;
ring Y is —C₃-C₆ cycloalkylene, 3- to 6-membered heterocycloalkylene having 1-2 ring heteroatoms independently selected from N, O, and S, phenylene, or 5- to 6-membered heteroarylene having 1-2 ring heteroatoms independently selected from N, O, and S;
m is 0, 1, 2, or 3;
each R³ when present, is independently halo, —CN, —C₁-C₆ alkyl, —C₁-C₆ haloalkyl, —C(O)OR^{3a}, —C(O)NR^{3b}R^{3c}, or —C₁-C₆ alkoxy;
R^{3a} is H or C₁-C₃ alkyl;
R^{3b} and R^{3c} are each independently —H or C₁-C₃ alkyl;
R⁴ is —C(O)NR^{4a}R^{4b}, or a 5- to 6-membered heteroaryl having 1 to 4 ring heteroatoms independently selected from N, O, and S, wherein said 5- to 6-membered heteroaryl is unsubstituted or substituted with 1-3 R^{4c};
R^{4a} and R^{4b} are each independently H, —C₁-C₆ alkyl, —C₁-C₆ haloalkyl, phenyl, or —(C₁-C₃ alkylene)-O—(C₁-C₃ alkyl); or
R^{4a} and R^{4b} taken together with the N atom to which they are attached form a 4- to 8-membered heterocycloalkyl having 0-1 additional ring heteroatom selected from N, O, and S; wherein said 4- to 8-membered heterocycloalkyl is unsubstituted or substituted with 1-3 substituents independently selected from the group consisting of halo, —CN, —C₁-C₆ alkyl, and —C₁-C₆ alkoxy; and
each R^{4c} is independently selected from the group consisting of —CN, —C₁-C₃ alkyl, —C₁-C₃ haloalkyl, and —C₁-C₃ hydroxyalkyl.

In some embodiments, R¹ is selected from the group consisting of —C₁-C₆ hydroxyalkyl, -(Q¹)-NR^{1a}R^{1b}, -(Q¹)-(C₃-C₇ cycloalkyl), -(Q¹)-phenyl, -(Q¹)-(5- to 6-membered heteroaryl), and -(Q¹)-(4- to 8-membered heterocycloalkyl); wherein said 5- to 6-membered heteroaryl has 1-3 ring heteroatoms independently selected from N, O, and S; said 4- to 8-membered heterocycloalkyl has 1-3 ring heteroatom or heteroatom groups independently selected from N, O, S, and S(O)₂; and said C₃-C₇ cycloalkyl, phenyl, 4- to 8-membered heterocycloalkyl, and 5- to 6-membered heteroaryl are unsubstituted or substituted with 1-2 substituents independently selected from —C₁-C₃ alkyl, —C₁-C₃ alkoxy, and NH₂.

In some embodiments, R¹ is —H. In some embodiments, R¹ is —C₁-C₆ haloalkyl. In some embodiments, R¹ is —C₁-C₆ hydroxyalkyl. In some embodiments, R¹ is —C(O)NH₂. In some embodiments, R¹ is —C(O)—(C₁-C₆-alkyl). In some embodiments, R¹ is -(Q¹)-NR^{1a}R^{1b}. In some embodiments, R¹ is -(Q¹)-(C₃-C₇ cycloalkyl), wherein said C₃-C₇ cycloalkyl is unsubstituted or substituted with 1-2 substituents independently selected from halo, —OH, —C₁-C₃ alkyl, —C₁-C₃ alkoxy, and NH₂. In some embodiments, R¹ is -(Q¹)-phenyl, wherein said phenyl is unsubstituted or substituted with 1-2 substituents independently selected from halo, —OH, —C₁-C₃ alkyl, —C₁-C₃ alkoxy, and NH₂. In some embodiments, R¹ is -(Q¹)-(5- to 6-membered heteroaryl), wherein said 5- to 6-membered heteroaryl has 1-3 ring heteroatoms independently selected from N, O, and S, and said 5- to 6-membered heteroaryl is unsubstituted or substituted with 1-2 substituents independently selected from halo, —OH, —C₁-C₃ alkyl, —C₁-C₃ alkoxy, and NH₂. In some embodiments, R¹ is -(Q¹)-(5- to 6-membered heteroaryl), wherein said 5- to 6-membered heteroaryl has 1-3 ring heteroatoms independently selected from N, and O, and said 5- to 6-membered heteroaryl is unsubstituted or substituted with 1-2 substituents independently selected from halo, —OH, —C₁-C₃ alkyl, —C₁-C₃ alkoxy, and NH₂. In some embodiments, R¹ is -(Q¹)-(4- to 8-membered heterocycloalkyl), wherein said 4- to 8-membered heterocycloalkyl has 1-3 ring heteroatom or heteroatom groups independently selected from N, O, S, and S(O)₂, and wherein said 4- to 8-membered heterocycloalkyl is unsubstituted or substituted with 1-2 substituents independently selected from halo, —OH, —C₁-C₃ alkyl, —C₁-C₃ alkoxy, and NH₂. In some embodiments, R¹ is -(Q¹)-(4- to 8-membered heterocycloalkyl), wherein said 4- to 8-membered heterocycloalkyl has 1-3 ring heteroatom or heteroatom groups independently selected from N, and O, and wherein said 4- to 8-membered heterocycloalkyl is unsubstituted or substituted with 1-2 substituents independently selected from halo, —OH, —C₁-C₃ alkyl, —C₁-C₃ alkoxy, and NH₂.

In some embodiments, R¹ is —(CH₂)—NR^{1a}R^{1b}, or —(CH₂)-(4- to 8-membered heterocycloalkyl), wherein said 4- to 8-membered heterocycloalkyl has 1-2 ring N atoms, and is unsubstituted or substituted with 1-2 substituents independently selected from —C₁-C₃ alkyl and —C₁-C₃ alkoxy;
R^{1a} and R^{1b} are independently selected from the group consisting of —H, —C₁-C₆ alkyl, —C₁-C₆ haloalkyl, —(C₁-C₃ alkylene)-O—(C₁-C₃ alkyl), —C₃-C₆ cycloalkyl, —(C₁-C₃ alkylene)-(C₃-C₆ cycloalkyl), 4- to 8-membered heterocycloalkyl having 1-3 ring heteroatoms independently selected from N, O, and S, and —(C₁-C₃ alkylene)-(4- to 8-membered heterocycloalkyl) wherein said 4- to 8-membered heterocycloalkyl has 1-2 ring heteroatoms independently selected from N and O; wherein said —(C₁-C₃ alkylene)-O—(C₁-C₃ alkyl), —C₃-C₆ cycloalkyl, —(C₁-C₃ alkylene)-(C₃-C₆ cycloalkyl), 4- to 8-membered heterocycloalkyl, and —(C₁-C₃ alkylene)-(4- to 8-membered heterocycloalkyl) are unsubstituted or substituted with 1-3 R^{1c}; and
each R^{1c}, when present, is independently halo, —C₁-C₃ alkyl, or —(C₁-C₃ alkylene)-O—(C₁-C₃ alkyl).

In some embodiments, R¹ is —(CH₂)—NR^{1a}R^{1b}, or —(CH₂)-(4- to 8-membered heterocycloalkyl), wherein said 4- to 8-membered heterocycloalkyl has 1-2 ring N atoms, and is unsubstituted or substituted with 1-2 substituents independently selected from —C₁-C₃ alkyl and —C₁-C₃ alkoxy;
R^{1a} and R^{1b} are independently selected from the group consisting of —H, —C₁-C₆ alkyl, —C₁-C₆ haloalkyl, —(C₁-C₃ alkylene)-O—(C₁-C₃ alkyl), —C₃-C₆ cycloalkyl, —(C₁-C₃ alkylene)-(C₃-C₆ cycloalkyl), and 4- to 8-membered heterocycloalkyl having 1-3 ring heteroatoms independently selected from N, O, and S, wherein said —(C₁-C₃ alkylene)-O—(C₁-C₃ alkyl), —C₃-C₆ cycloalkyl, —(C₁-C₃ alkylene)-(C₃-C₆ cycloalkyl), and 4- to 8-membered heterocycloalkyl are unsubstituted or substituted with 1-3 R^{1c}; and
each R^{1c}, when present, is independently halo, —C₁-C₃ alkyl, or —(C₁-C₃ alkylene)-O—(C₁-C₃ alkyl).

In some embodiments, R¹ is —(CH₂)—NR^{1a}R^{1b}; and one of R^{1a} and R^{1b} is —H.

In some embodiments, R¹ is —(CH₂)-(4- to 6-membered heterocycloalkyl); wherein said 4- to 6-membered heterocycloalkyl has 1-2 ring N atoms and is unsubstituted or substituted with a —C₁-C₃ alkyl.

In some embodiments, R¹ is selected from the group consisting of:

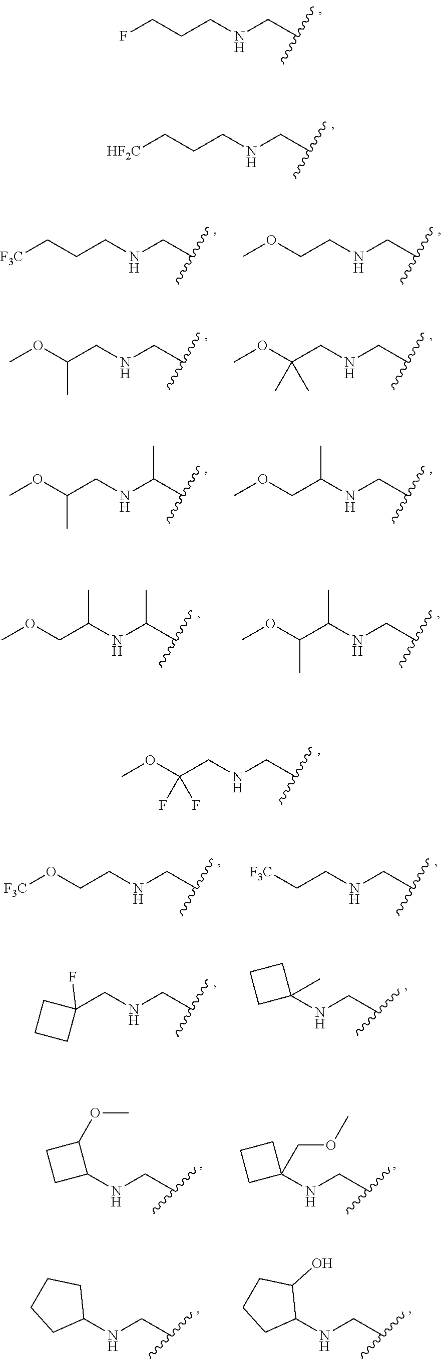

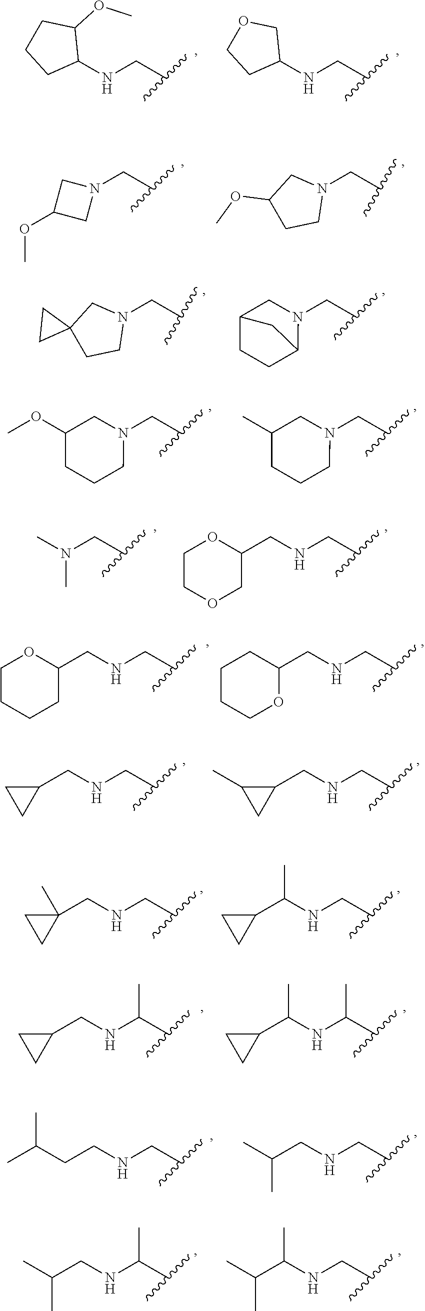

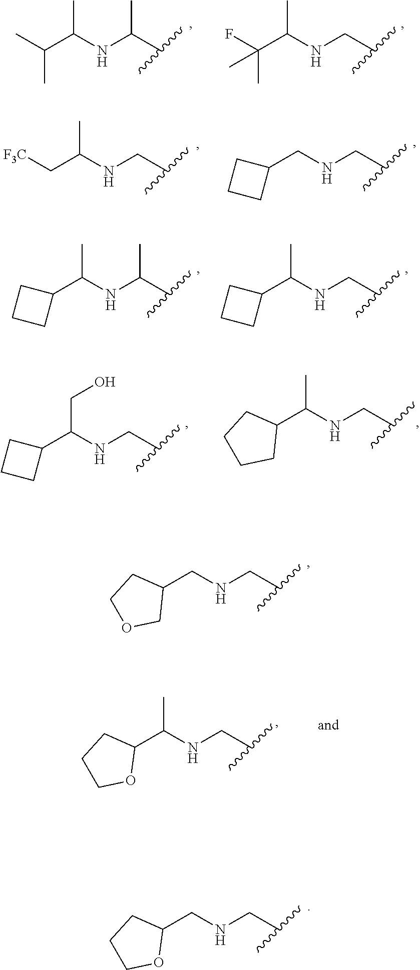

In some embodiments, R¹ is selected from the group consisting of:

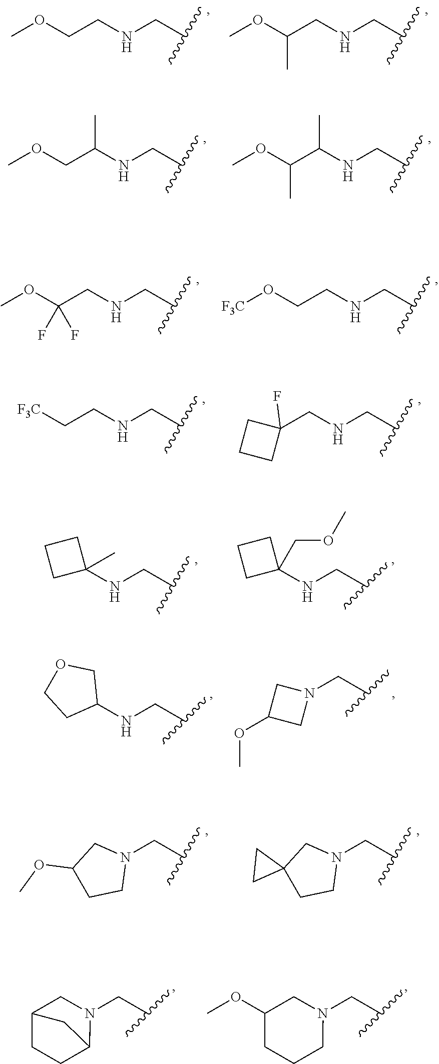

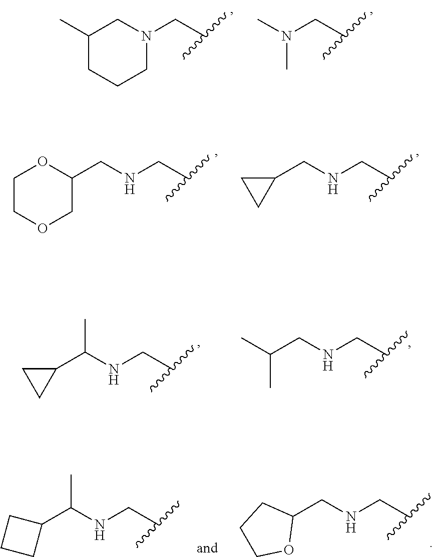

In some embodiments, R¹ is selected from the group consisting of:

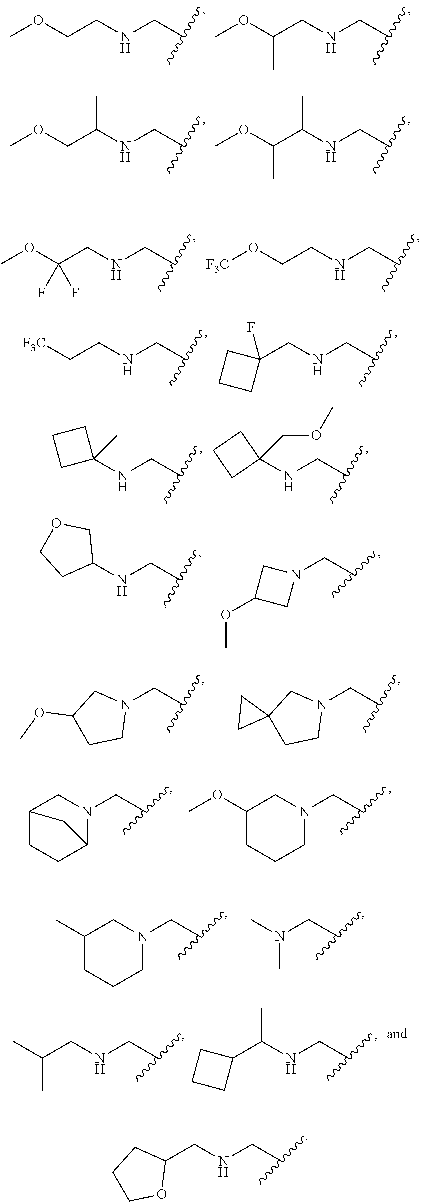

In some embodiments, R¹ is selected from the group consisting of:

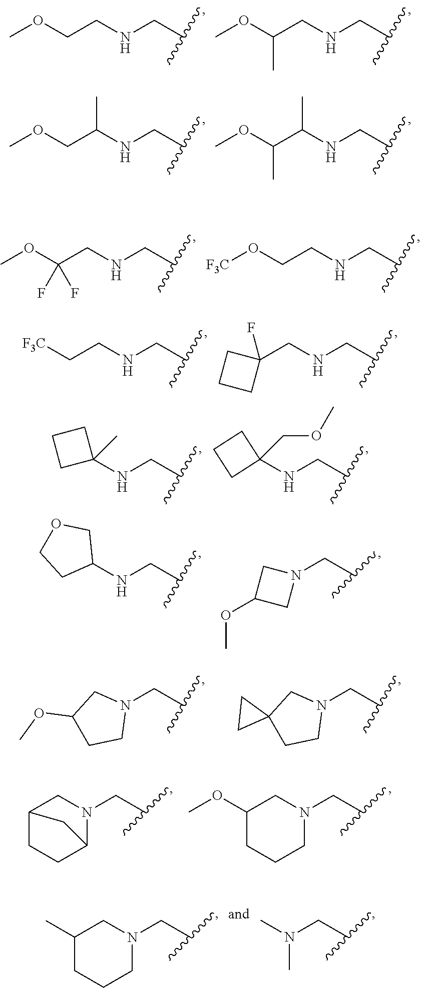

In some embodiments, R¹ is selected from the group consisting of:

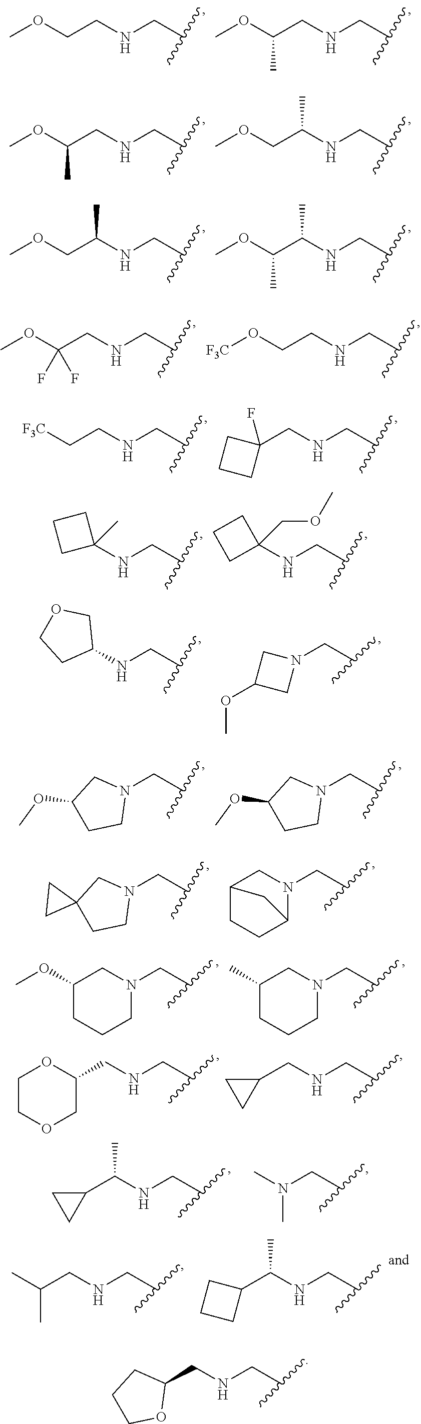

In some embodiments, R¹ is selected from the group consisting of:

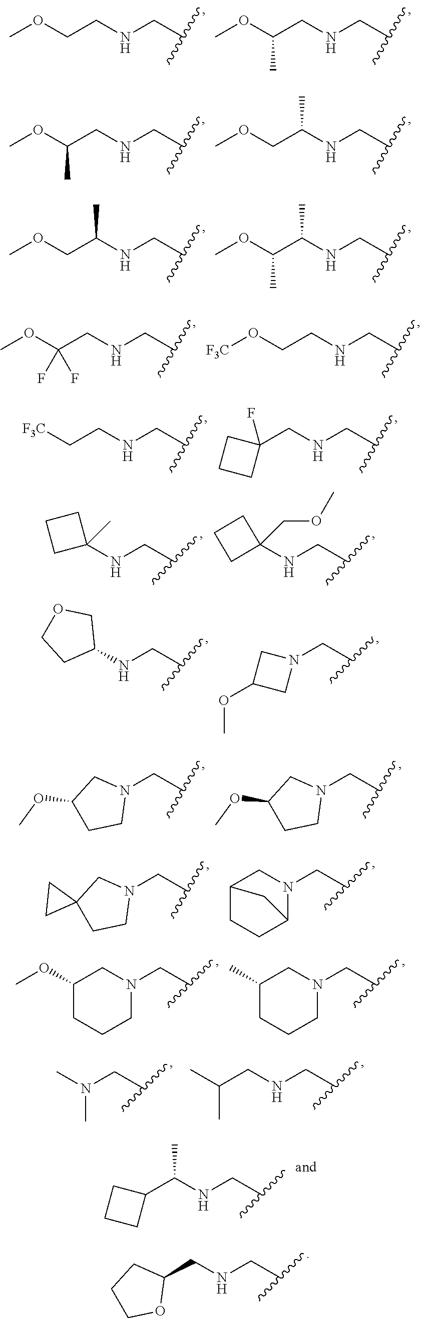

In some embodiments, R¹ is selected from the group consisting of:

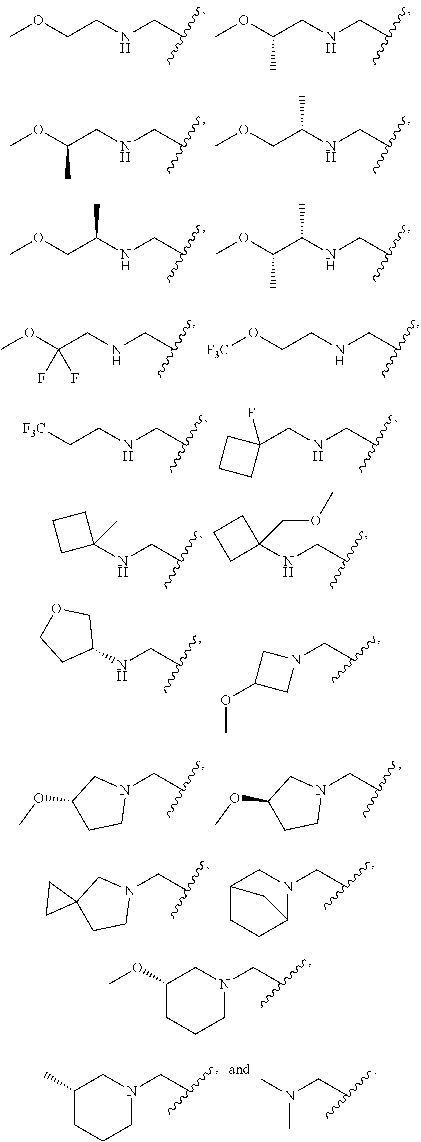

In some embodiments, Q¹ is absent. In some embodiments, Q¹ is unsubstituted —(C₁-C₃ alkylene)-. In some embodiments, Q¹ is —(C₁-C₃ alkylene)- substituted with 1-3 R^{q}. In some embodiments, Q¹ is —(CH₂)—.

In some embodiments, R^{1a} and R^{1b} are independently selected from the group consisting of —H, —C₁-C₆ alkyl, —C₁-C₆ haloalkyl, —(C₁-C₆ alkylene)-O—(C₁-C₃ alkyl), —C₃-C₆ cycloalkyl, —(C₁-C₃ alkylene)-(C₃-C₆ cycloalkyl), and 4- to 8-membered heterocycloalkyl having 1-3 ring heteroatom or heteroatom groups independently selected from N, O, S, and S(O)₂; wherein said phenyl, —(C₁-C₆ alkylene)-O—(C₁-C₃ alkyl), —C₃-C₆ cycloalkyl, —(C₁-C₃ alkylene)-(C₃-C₆ cycloalkyl), and 4- to 8-membered heterocycloalkyl are unsubstituted or substituted with 1-3 R^{1c}.

In some embodiments, R^{1a} and R^{1b} are independently selected from the group consisting of —H, —C₁-C₄ alkyl, —C₁-C₄ haloalkyl, —(C₁-C₄ alkylene)-O—(C₁-C₃ alkyl), —C₃-C₄ cycloalkyl, —(C₁-C₃ alkylene)-(C₃-C₆ cycloalkyl), 4- to 8-membered heterocycloalkyl having 1-3 ring heteroatoms independently selected from N, O, and S, and —(C₁-C₃ alkylene)-(4- to 8-membered heterocycloalkyl) wherein said 4- to 8-membered heterocycloalkyl has 1-3 ring heteroatoms independently selected from N, O, and S; wherein said —(C₁-C₆ alkylene)-O—(C₁-C₃ alkyl), —C₃-C₆ cycloalkyl, —(C₁-C₃ alkylene)-(C₃-C₆ cycloalkyl), 4- to 8-membered heterocycloalkyl, and —(C₁-C₃ alkylene)-(4- to 8-membered heterocycloalkyl) are unsubstituted or substituted with 1-3 R^{1c}. In some embodiments, R^{1a} and R^{1b} are independently selected from the group consisting of —H, —C₁-C₄ alkyl, —C₁-C₄ haloalkyl, —(C₁-C₄ alkylene)-O—(C₁-C₃ alkyl), —C₃-C₄ cycloalkyl, —(C₁-C₃ alkylene)-(C₃-C₆ cycloalkyl), 4- to 8-membered heterocycloalkyl having 1-3 ring heteroatoms independently selected from N and O, and —(C₁-C₃ alkylene)-(4- to 8-membered heterocycloalkyl) wherein said 4- to 8-membered heterocycloalkyl has 1-3 ring heteroatoms independently selected from N, O, and S; wherein said —(C₁-C₆ alkylene)-O—(C₁-C₃ alkyl), —C₃-C₆ cycloalkyl, —(C₁-C₃ alkylene)-(C₃-C₆ cycloalkyl), 4- to 8-membered heterocycloalkyl, and —(C₁-C₃ alkylene)-(4- to 8-membered heterocycloalkyl) are unsubstituted or substituted with 1-3 R^{1c}.

In some embodiments, one of R^{1a} and R^{1b} is H, and the other is selected from the group consisting of —H, —C₁-C₄ alkyl, —C₁-C₄ haloalkyl, —(C₁-C₄ alkylene)-O—(C₁-C₃ alkyl), —C₃-C₄ cycloalkyl, —(C₁-C₃ alkylene)-(C₃-C₆ cycloalkyl), 4- to 8-membered heterocycloalkyl having 1-3 ring heteroatoms independently selected from N, O, and S, and —(C₁-C₃ alkylene)-(4- to 8-membered heterocycloalkyl) wherein said 4- to 8-membered heterocycloalkyl has 1-3 ring heteroatoms independently selected from N, O, and S; wherein said —(C₁-C₆ alkylene)-O—(C₁-C₃ alkyl), —C₃-C₆ cycloalkyl, —(C₁-C₃ alkylene)-(C₃-C₆ cycloalkyl), 4- to 8-membered heterocycloalkyl, and —(C₁-C₃ alkylene)-(4- to 8-membered heterocycloalkyl) are unsubstituted or substituted with 1-3 R^{1c}. In some embodiments, one of R^{1a} and R^{1b} is H, and the other is selected from the group consisting of —H, —C₁-C₄ alkyl, —C₁-C₄ haloalkyl, —(C₁-C₄ alkylene)-O—(C₁-C₃ alkyl), —C₃-C₄ cycloalkyl, —(C₁-C₃ alkylene)-(C₃-C₆ cycloalkyl), 4- to 8-membered heterocycloalkyl having 1-3 ring heteroatoms independently selected from N, and O, and —(C₁-C₃ alkylene)-(4- to 8-membered heterocycloalkyl) wherein said 4- to 8-membered heterocycloalkyl has 1-3 ring heteroatoms independently selected from N, O, and S; wherein said —(C₁-C₆ alkylene)-O—(C₁-C₃ alkyl), —C₃-C₆ cycloalkyl, —(C₁-C₃ alkylene)-(C₃-C₆ cycloalkyl), 4- to 8-membered heterocycloalkyl, and —(C₁-C₃ alkylene)-(4- to 8-membered heterocycloalkyl) are unsubstituted or substituted with 1-3 R^{1c}

In some embodiments, each R^{1c}, when present, is independently halo. In some embodiments, each R^{1c}, when present, is independently —OH. In some embodiments, each R^{1c}, when present, is independently —C₁-C₃ alkyl. In some embodiments, each R^{1c}, when present, is independently —C₁-C₃ hydroxyalkyl. In some embodiments, each R^{1c}, when present, is independently —C₁-C₃ haloalkyl. In some embodiments, each R^{1c}, when present, is independently —C₁-C₃ alkoxy. In some embodiments, each R^{1c}, when present, is independently —(C₁-C₃ alkylene)-O—(C₁-C₃ alkyl). In some embodiments, each R^{1c}, when present, is independently halo, —C₁-C₃ alkyl, or —(C₁-C₃ alkylene)-O—(C₁-C₃ alkyl).

In some embodiments, R² is H. In some embodiments, R² is —C₁-C₆ alkyl. In some embodiments, R² is —C₂-C₆ alkenyl. In some embodiments, R² is —C₁-C₆ haloalkyl. In some embodiments, R² is —C₁-C₆ hydroxyalkyl. In some embodiments, R² is —C₃-C₈ cycloalkyl. In some embodiments, R² is —(C₁-C₃ alkylene)-(C₃-C₈-cycloalkyl). In some embodiments, R² is —(C₁-C₃ alkylene)-(3- to 8-membered heterocycloalkyl), wherein said 3- to 8-membered heterocycloalkyl has 1-3 ring heteroatoms independently selected from N, O, and S. In some embodiments, R² is —(C₁-C₃ alkylene)-(3- to 6-membered heterocycloalkyl), wherein said 3- to 6-membered heterocycloalkyl has 1-3 ring heteroatoms independently selected from N, and O.

In some embodiments, R² is —H, —C₁-C₆ alkyl, —C₂-C₆ alkenyl, —C₁-C₆ haloalkyl, —C₁-C₆ hydroxyalkyl, —C₃-C₈ cycloalkyl, —(C₁-C₃ alkylene)-(C₃-C₈-cycloalkyl), or —(C₁-C₃ alkylene)-(3- to 8-membered heterocycloalkyl), wherein said 3- to 8-membered heterocycloalkyl has 1-3 ring heteroatoms independently selected from N, O, and S. In some embodiments, R² is —C₁-C₆ alkyl, —C₁-C₆ haloalkyl, —C₁-C₆ hydroxyalkyl, —C₃-C₈ cycloalkyl, —(C₁-C₃ alkylene)-(C₃-C₈-cycloalkyl), or —(C₁-C₃ alkylene)-(3- to 8-membered heterocycloalkyl), wherein said 3- to 8-membered heterocycloalkyl has 1-3 ring heteroatoms independently selected from N, O, and S.

In some embodiments, R² is —C₁-C₆ alkyl, —C₁-C₆ haloalkyl, or —C₃-C₈ cycloalkyl.

In some embodiments, R² is selected from the group consisting of: H,

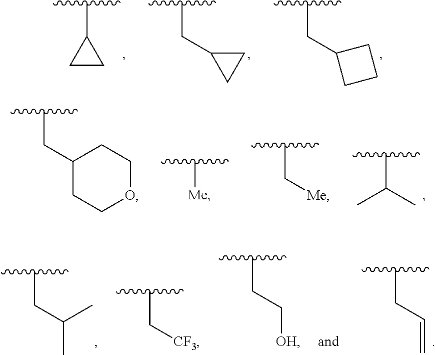

In some embodiments, R² is selected from the group consisting of: H,

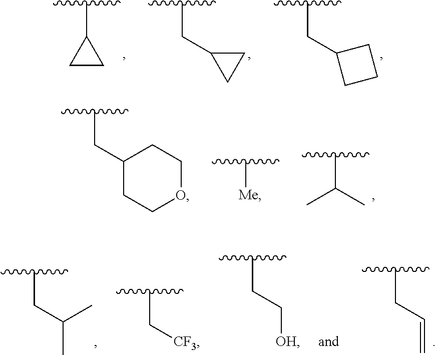

In some embodiments, R² is selected from the group consisting of:

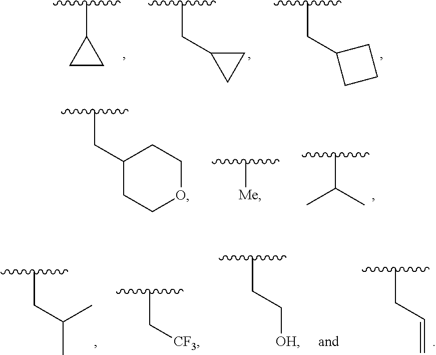

In some embodiments, R² is selected from the group consisting of:

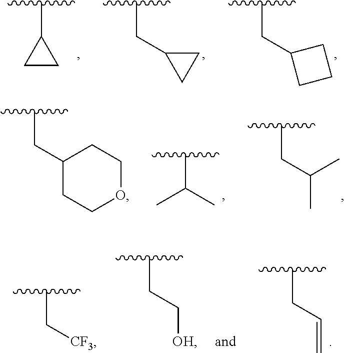

In some embodiments, R² is

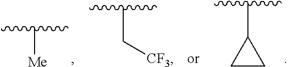

In some embodiments, R² is

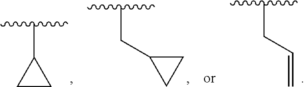

In some embodiments, R² is cyclopropyl.

In some embodiments, each R³, when present, is halo, —CN, —C₁-C₆ alkyl, —C(O)OR^{3a}, —C(O)NH₂, or —C₁-C₆ alkoxy. In some embodiments, each R³, when present, is independently selected from: a) halo, —CN, —C(O)OR^{3a}, —C(O)NH₂, and —C₁-C₆ haloalkoxy; and b) halo, —C₁-C₃ alkyl, and —C₁-C₃ alkoxy. In some embodiments, each R³, when present, is independently halo, —CN, —C(O)OR^{3a}, —C(O)NH₂ or —C₁-C₆ haloalkoxy. In some embodiments, each R³, when present, is independently halo, —C(O)OR^{3a}, —C(O)NH₂ or —C₁-C₆ haloalkoxy. In some embodiments, each R³, when present, is independently: a) halo, —CN, —C(O)OR^{3a}, or —C(O)NH₂; or b) halo, —C₁-C₃ alkyl, or —C₁-C₃ alkoxy. In some embodiments, each R³, when present, is independently halo, —CN, —C(O)OR^{3a}, or —C(O)NH₂. In some embodiments, each R³, when present, is independently halo, —C₁-C₃ alkyl, or —C₁-C₃ alkoxy. In some embodiments, each R³, when present, is independently halo, —CN, or —C₁-C₃ haloalkoxy. In some embodiments, each R³, when present, is independently halo, —CN, or —OCF₂H. In some embodiments, each R³, when present, is independently halo or —CN. In some embodiments, each R³, when present, is independently —F, —Cl, —CN, —CH₃, —OCH₃, —OCF₂H, —C(O)OCH₃, or —C(O)NH₂. In some embodiments, each R³, when present, is independently —F, —Cl, —CN, —OCF₂H, —C(O)OCH₃, or —C(O)NH₂. In some embodiments, each R³, when present, is independently —F, —Cl, —CN, or —OCF₂H. In some embodiments, each R³, when present, is independently F, —CN, —C(O)OCH₃, or —C(O)NH₂. In some embodiments, each R³, when present, is independently F, —CH₃, or —OCH₃.

In some embodiments, each R³, when present, is independently halo. In some embodiments, each R³, when present, is independently —CN. In some embodiments, each R³, when present, is independently —C₁-C₆ alkyl. In some embodiments, each R³, when present, is independently —C₁-C₆ haloalkyl. In some embodiments, each R³, when present, is independently —C(O)OR^{3a}. In some embodiments, each R³, when present, is independently —C(O)NR^{3b}R^{3c}. In some embodiments, each R³, when present, is independently —C₁-C₆ alkoxy. In some embodiments each R³, when present, is independently —C₁-C₆ haloalkoxy. In some embodiments, R³ is halo or —CN.

In some embodiments, m is 1, and R³ is halo, —CN, —C(O)OR^{3a}, —C(O)NH₂, or —C₁-C₆ haloalkoxy. In some embodiments, m is 1, and R³ is halo, —CN, —C(O)OR^{3a}, or —C(O)NH₂. In some embodiments, m is 1, and R³ is —C₁-C₃ alkyl or —C₁-C₃ alkoxy. In some embodiments, m is 2, and each R³ is independently halo. In some embodiments, m is 1 or 2, and each R³ is independently halo, —CN, or —C₁-C₃ haloalkoxy.

In some embodiments, R^{3a} is H. In some embodiments, R^{3a} is C₁-C₃ alkyl.

In some embodiments, R^{3b} and R^{3c} are each independently —H. In some embodiments, R^{3b} and R^{3c} are each independently C₁-C₃ alkyl. In some embodiments, R^{3b} is —H, and R^{3c} is C₁-C₃ alkyl.

In some embodiments, X¹ is CH. In some embodiments, X¹ is N. In some embodiments, X² is CH. In some embodiments, X² is N. In some embodiments, X³ is CH. In some embodiments, X³ is N. In some embodiments, X⁴ is CR^{X}. In some embodiments, X⁴ is N. In some embodiments, X¹ is CH or N, X² and X³ are CH, and X⁴ is CR^{X}. In some embodiments, X¹ is CH, X² and X³ are CH, and X⁴ is CR^{X}. In some embodiments, X¹ is N, X² and X³ are CH, and X⁴ is CR^{X}.

In some embodiments, R^{X} is —H, halo, —CN, —C₁-C₆ alkoxy, —NH₂, or —C₃-C₈ cycloalkyl. In some embodiments, R^{X}, when present, is —C₃-C₈ cycloalkyl. In some embodiments, R^{X}, when present, is —H, —Cl, —CN, —OCH₃, —NH₂, or cyclopropyl.

In some embodiments, R^{X} is halo, —CN, —C₁-C₆ alkoxy, —C₁-C₆ haloalkoxy, —NR^{Xa}R^{Xb}, or —C₃-C₈ cycloalkyl. In some embodiments, R^{X} is halo, —C₁-C₆ alkoxy, —NH₂, or —C₃-C₈ cycloalkyl. In some embodiments, R^{X} is —Cl, —CN, —OCH₃, —OCH₂CH₃, —OCH(CH₃)₂, —OCH₂F, —OCH₂CF₃, —OCH₂CF₂H, —NH₂, —N(CH₃)₂, —NH(CH(CH₃)₂), —NHCH₂CH₃, or cyclopropyl. In some embodiments, R^{X} is —Cl, —CN, —OCH₂CH₃, —OCH₂CF₃, —OCH₂CF₂H, —NH₂, —NH(CH(CH₃)₂), or cyclopropyl. In some embodiments, R^{X} is —Cl, —OCH₂CH₃, —OCH₂CF₃, —NH₂, —NH(CH(CH₃)₂), or cyclopropyl. In some embodiments, R^{X}, when present, is —H. In some embodiments, R^{X}, when present, is halo. In some embodiments, R^{X}, when present, is —CN. In some embodiments, R^{X}, when present, is —OH. In some embodiments, R^{X}, when present, is —C₁-C₆ alkyl.

In some embodiments, R^{X} when present is methyl. In some embodiments, R^{X}, when present, is —C₁-C₆ haloalkyl. In some embodiments, R^{X}, when present, is —C₁-C₆ haloalkoxy. In some embodiments, R^{X}, when present, is —C₁-C₆ alkoxy. In some embodiments, R^{X}, when present, is —NR^{Xa}R^{Xb}. In further embodiments, R^{X} is —C₃-C₆ cycloalkoxy. In some embodiments, R^{X} is cyclopropoxy.

In some embodiments, R^{Xa} and R^{Xb} are independently —H. In some embodiments, R^{Xa} and R^{Xb} are independently —C₁-C₃ alkyl. In some embodiments, R^{Xa} is —H, and R^{Xb} is —C₁-C₃ alkyl. In some embodiments, R^{Xa} is —H, and R^{Xb} is —(C₁-C₃ alkylene)-NR^{Xc}R^{Xd}.

In some embodiments, R^{Xc} and R^{Xd} are independently —H. In some embodiments, R^{Xc} and R^{Xd} are independently —C₁-C₃ alkyl. In some embodiments, R^{Xc} is —H, and R^{Xd} is —C₁-C₃ alkyl.

In some embodiments, ring Y is —C₃-C₆ cycloalkylene, or phenylene. In some embodiments, ring Y is —C₃-C₆ cycloalkylene. In some embodiments, ring Y is cyclobutylene. In some embodiments, ring Y is 3- to 6-membered heterocycloalkylene having 1-2 ring heteroatoms independently selected from N, O, and S. In some embodiments, ring Y is oxetanylene. In some embodiments, ring Y is phenylene. In some embodiments, ring Y is 5- to 6-membered heteroarylene having 1-2 ring heteroatoms independently selected from N, O, and S. In some embodiments, ring Y is 5- to 6-membered heteroarylene having 1-2 ring heteroatoms independently selected from N, and O.

In some embodiments, m is 0, 1, 2, or 3. In some embodiments, m is 1 or 2. In some embodiments, m is 0. In some embodiments, m is 1. In some embodiments, m is 2. In some embodiments, m is 3.

In some embodiments, R⁴ is —C(O)NR^{4a}R^{4b}, or a 5- to 6-membered heteroaryl having 1 to 3 heteroatoms independently selected from N, O, and S, wherein said 5- to 6-membered heteroaryl is unsubstituted or substituted with 1-3 R^{4c}.

In some embodiments, R⁴ is —C(O)NR^{4a}R^{4b}.

In some embodiments, R^{4a} and R^{4b} are each independently H, —C₁-C₆ alkyl, —C₁-C₆ haloalkyl, phenyl, or —(C₁-C₃ alkylene)-O—(C₁-C₃ alkyl). In some embodiments, R^{4a} and R^{4b} are methyl.

In some embodiments, R^{4a} and R^{4b} taken together with the N atom to which they are attached form a 4- to 8-membered heterocycloalkyl having 0-1 additional ring heteroatom selected from N, O, and S; wherein said 4- to 8-membered heterocycloalkyl is unsubstituted or substituted with 1-3 substituents independently selected from the group consisting of halo, —CN, —C₁-C₆ alkyl, and —C₁-C₆ alkoxy. In some embodiments, R^{4a} and R^{4b} are taken together with the N atom to which they are attached to form a 4- to 8-membered heterocycloalkyl having 0-1 additional ring heteroatom selected from N, O, and S; wherein said 4- to 8-membered heterocycloalkyl is unsubstituted or substituted with 1-3 halo. In some embodiments, R^{4a} and R^{4b} are taken together with the N atom to which they are attached to form an azetidine ring, wherein said azetidine ring is unsubstituted or substituted with 1-2 substituents independently selected from halo, —CN, and —C₁-C₆ alkyl. In some embodiments, R⁴ is

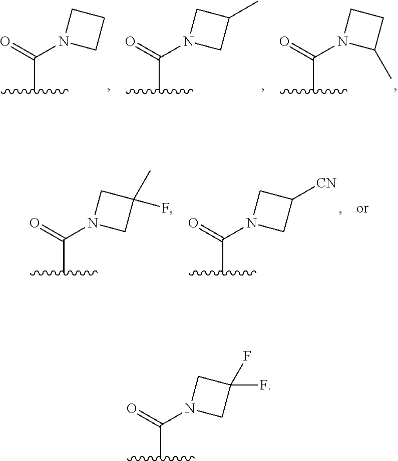

In one embodiment, R⁴ is

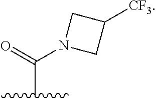 [SMILES: *C(=O)N1CC(C(F)(F)F)C1]

In some embodiments, R^{4a} and R^{4b} are taken together with the N atom to which they are attached to form an azetidine ring, wherein said azetidine ring is substituted with 1-2 halo. In some embodiments, R⁴ is

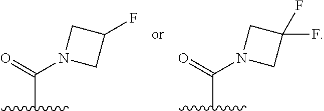 [SMILES: *C(=O)N1CC(F)C1 ; *C(=O)N1CC(F)(F)C1]

In some embodiments R⁴ is a 5- to 6-membered heteroaryl having 1 to 4 ring heteroatoms independently selected from N, O, and S, wherein said 5- to 6-membered heteroaryl is unsubstituted or substituted with 1-3 R^{4c}. In some embodiments, R⁴ is a 5- to 6-membered heteroaryl having 1-3 ring heteroatoms independently selected from N, and O, wherein said heteroaryl is substituted with 1-3 R^{4c}.

In some embodiments, R⁴ is triazolyl, oxazolyl, imidazolyl, pyrazolyl, pyridinyl, or pyridazinyl, each of which is substituted with 1-3 R^{4c4}. In some embodiments, R⁴ is selected from the group consisting of:

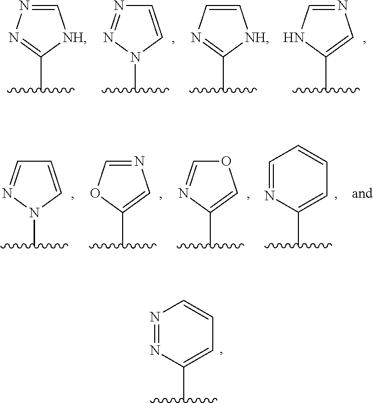

each of which is substituted with 1-2 R^{4c}. In some embodiments, R⁴ is selected from the group consisting of:

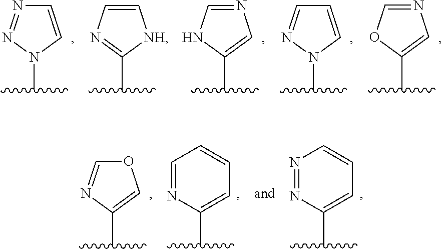

each of which is substituted with 1-2 R^{4c}. In some embodiments, R⁴ is

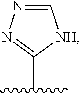 [SMILES: *c1nnc[nH]1]

which is substituted with 1-2 R^{4c}.

In some embodiments, each R^{4c} is independently selected from the group consisting of —CN, —C₁-C₃ alkyl, and —C₁-C₃ haloalkyl.

In some embodiments, each R^{4c} is independently —CN. In some embodiments, each R^{4c} is independently —C₁-C₃ alkyl. In some embodiments, each R^{4c} is independently —C₁-C₃ haloalkyl. In some embodiments, each R^{4c} is independently —C₁-C₃ hydroxyalkyl. In some embodiments, each R^{4c} is independently —CH₃, —CH₂F, —CF₂H, —CF₃, or —CN. In some embodiments, each R^{4c} is —CH₃.

In some embodiments, the compound, or pharmaceutically acceptable salt thereof, has a structure according to Formula Ia:

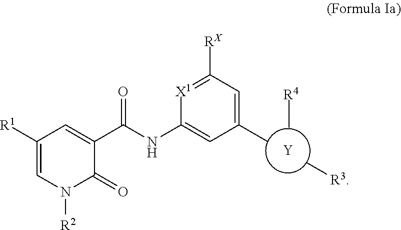

In some embodiments of a compound having a structure according to Formula Ia,
R¹ is selected from the group consisting of —C₁-C₆ hydroxyalkyl, -(Q¹)-NR^{1a}R^{1b}, -(Q¹)-(C₃-C₇ cycloalkyl), -(Q¹)-phenyl, -(Q¹)-(5- to 6-membered heteroaryl), and -(Q¹)-(4- to 8-membered heterocycloalkyl); wherein said 5- to 6-membered heteroaryl and 4- to 8-membered heterocycloalkyl have 1-3 ring heteroatoms independently selected from N, O, and S; and wherein said phenyl, 4- to 8-membered heterocycloalkyl, and 5- to 6-membered heteroaryl are unsubstituted or substituted with 1-2 substituents independently selected from the group consisting of —C₁-C₃ alkyl, —C₁-C₃ alkoxy, and —NH₂;
Q¹ is absent, or unsubstituted —(C₁-C₃ alkylene)-;
R^{1a} and R^{1b} are independently selected from the group consisting of —H, —C₁-C₆ alkyl, —C₁-C₆ haloalkyl, —(C₁-C₃ alkylene)-O—(C₁-C₃ alkyl), —C₃-C₆ cycloalkyl, —(C₁-C₃ alkylene)-(C₃-C₆ cycloalkyl), and 4- to 8-membered heterocycloalkyl having 1-3 ring heteroatoms independently selected from N, O, and S, wherein said —(C₁-C₃ alkylene)-O—(C₁-C₃ alkyl), —C₃-C₆ cycloalkyl, —(C₁-C₃ alkylene)-(C₃-C₆ cycloalkyl), and 4- to 8-membered heterocycloalkyl are unsubstituted or substituted with 1-3 R^{1c};
each R^{1c}, when present, is independently halo, —C₁-C₃ alkyl, or —(C₁-C₃ alkylene)-O—(C₁-C₃ alkyl);
R² is —H, —C₁-C₆ alkyl, —C₂-C₆ alkenyl, —C₁-C₆ haloalkyl, —C₁-C₆ hydroxyalkyl, —C₃-C₈ cycloalkyl, —(C₁-C₃ alkylene)-(C₃-C₈ cycloalkyl), or —(C₁-C₃ alkylene)-(3- to 8-membered heterocycloalkyl), wherein said 3- to 8-membered heterocycloalkyl has 1-3 ring heteroatoms independently selected from N, O, and S;
X¹ is N or CH;
R^{X} is —H, halo, —CN, —C₁-C₆ alkoxy, —NH₂, or —C₃-C₈ cycloalkyl;
ring Y is —C₃-C₆ cycloalkylene, or phenylene;
R³ is halo, —CN, —C₁-C₆ alkyl, —C(O)OR^{3a}, —C(O)NH₂, or —C₁-C₆ alkoxy;
R^{3a} is C₁-C₃ alkyl;
R⁴ is —C(O)NR^{4a}R^{4b}, or a 5- to 6-membered heteroaryl having 1 to 3 heteroatoms independently selected from N, O, and S, wherein said 5- to 6-membered heteroaryl is unsubstituted or substituted with 1-3 R^{4c};
R^{4a} and R^{4b} taken together with the N atom to which they are attached form a 4- to 8-membered heterocycloalkyl having 0-1 additional ring heteroatom selected from N, O, and S; wherein said 4- to 8-membered heterocycloalkyl is unsubstituted or substituted with 1-3 halo; and
each R^{4c} is independently selected from the group consisting of —CN, —C₁-C₃ alkyl, and —C₁-C₃ haloalkyl.

In some embodiments, the compound, or pharmaceutically acceptable salt thereof, has a structure according to Formula Ib:

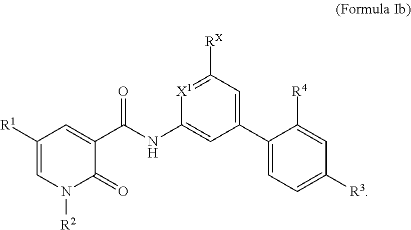

In some embodiments, the compound, or pharmaceutically acceptable salt thereof, has a structure according to Formula Ic:

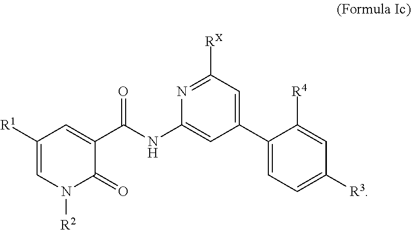

In some embodiments, the compound, or pharmaceutically acceptable salt thereof, has a structure according to Formula Ic:

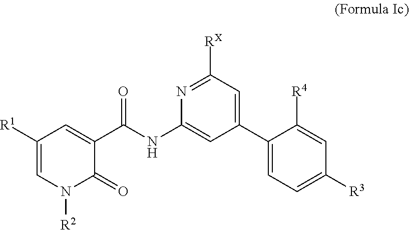

wherein:
R¹ is selected from the group consisting of —C₁-C₆ hydroxyalkyl, -(Q¹)-NR^{1a}R^{1b}, -(Q¹)-(C₃-C₇ cycloalkyl), -(Q¹)-phenyl, -(Q¹)-(5- to 6-membered heteroaryl), and -(Q¹)-(4- to 8-membered heterocycloalkyl); wherein said 5- to 6-membered heteroaryl and 4- to 8-membered heterocycloalkyl have 1-3 ring heteroatoms independently selected from N, O, and S; and wherein said phenyl, 4- to 8-membered heterocycloalkyl, and 5- to 6-membered heteroaryl are unsubstituted or substituted with 1-2 substituents independently selected from the group consisting of —C₁-C₃ alkyl, —C₁-C₃ alkoxy, and —NH₂;
Q¹ is absent, or unsubstituted —(C₁-C₃ alkylene)-;
R^{1a} and R^{1b} are independently selected from the group consisting of —H, —C₁-C₆ alkyl, —C₁-C₆ haloalkyl, —(C₁-C₃ alkylene)-O—(C₁-C₃ alkyl), —C₃-C₆ cycloalkyl, —(C₁-C₃ alkylene)-(C₃-C₆ cycloalkyl), and 4- to 8-membered heterocycloalkyl having 1-3 ring heteroatoms independently selected from N, O, and S, wherein said —(C₁-C₃ alkylene)-O—(C₁-C₃ alkyl), —C₃-C₆ cycloalkyl, —(C₁-C₃ alkylene)-(C₃-C₆ cycloalkyl), and 4- to 8-membered heterocycloalkyl are unsubstituted or substituted with 1-3 R^{1c};
each R^{1c}, when present, is independently halo, —C₁-C₃ alkyl, or —(C₁-C₃ alkylene)-O—(C₁-C₃ alkyl);
R² is —H, —C₁-C₆ alkyl, —C₂-C₆ alkenyl, —C₁-C₆ haloalkyl, —C₁-C₆ hydroxyalkyl, —C₃-C₈ cycloalkyl, —(C₁-C₃ alkylene)-(C₃-C₈ cycloalkyl), or —(C₁-C₃ alkylene)-(3- to 8-membered heterocycloalkyl), wherein said 3- to 8-membered heterocycloalkyl has 1-3 ring heteroatoms independently selected from N, O, and S;
R^{X} is halo, —C₁-C₆ alkoxy, —NH₂, or —C₃-C₈ cycloalkyl;
R³ is halo, —CN, —C(O)OR^{3a}, or —C(O)NH₂;
R^{3a} is C₁-C₃ alkyl;
R⁴ is 5- to 6-membered heteroaryl having 1 to 3 heteroatoms independently selected from N, O, and S, wherein said 5- to 6-membered heteroaryl is unsubstituted or substituted with 1-3 R^{4c}; and
each R^{4c} is independently selected from the group consisting of —CN, —C₁-C₃ alkyl, and —C₁-C₃ haloalkyl.

In some embodiments, the compound, or pharmaceutically acceptable salt thereof, has a structure according to Formula Id:

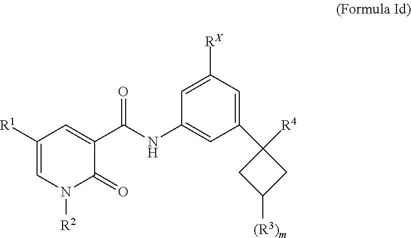

wherein m is 1 or 2.

In some embodiments, the compound, or pharmaceutically acceptable salt thereof, has a structure according to Formula Id:

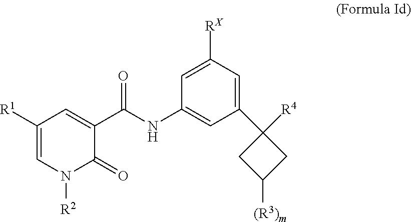

wherein:
R¹ is —(C₁-C₃ alkylene)-(4- to 8-membered heterocycloalkyl) having 1-3 ring heteroatoms independently selected from N, O, and S; and wherein said 4- to 8-membered heterocycloalkyl is substituted with 1-2 —C₁-C₃ alkyl;
R² is —C₂-C₆ alkenyl, —C₃-C₈ cycloalkyl, or —(C₁-C₃ alkylene)-(C₃-C₈-cycloalkyl);
R^{X} is —H;
m is 1 or 2;
each R³ is independently halo, —C₁-C₆ alkyl, or —C₁-C₆ alkoxy;
R⁴ is 5-membered heteroaryl having 1 to 3 heteroatoms independently selected from N, O, and S, wherein said 5-membered heteroaryl is substituted with 1-3 R^{4c}; and
each R^{4c} is independently —C₁-C₃ alkyl.

In some embodiments, the compound, or pharmaceutically acceptable salt thereof, has a structure according to according to Formula Ie:

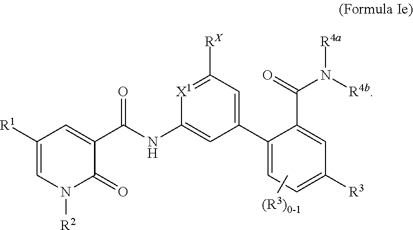

In some embodiments, the compound, or pharmaceutically acceptable salt thereof, has a structure according to according to Formula If:

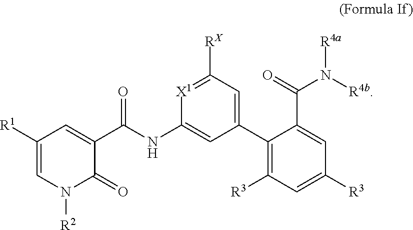

In some embodiments, the compound, or pharmaceutically acceptable salt thereof, has a structure according to according to Formula Ig:

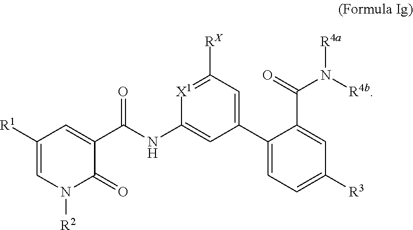

In some embodiments, the compound, or pharmaceutically acceptable salt thereof, has a structure according to Formula Ie:

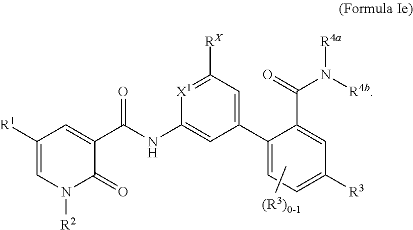

wherein:
R¹ is selected from the group consisting of -(Q¹)-NR^{1a}R^{1b} and -(Q¹)-(4- to 8-membered heterocycloalkyl) having 1-3 ring heteroatoms independently selected from N, O, and S; and wherein said 4- to 8-membered heterocycloalkyl is substituted with 1-2 —C₁-C₃ alkyl;
Q¹ is unsubstituted —(C₁-C₃ alkylene)-;
R^{1a} and R^{1b} are independently selected from the group consisting of —H, —C₁-C₆ alkyl, —(C₁-C₃ alkylene)-O—(C₁-C₃ alkyl), —(C₁-C₃ alkylene)-(C₃-C₆ cycloalkyl), and —(C₁-C₃ alkylene)-(4- to 8-membered heterocycloalkyl) wherein said 4- to 8-membered heterocycloalkyl has 1-2 ring heteroatoms independently selected from N and O;
R² is —C₁-C₆ alkyl, —C₁-C₆ haloalkyl, or —C₃-C₈ cycloalkyl;
X¹ is N or CH;
R^{X} is —CN, —C₁-C₆ alkoxy, —C₁-C₆ haloalkoxy, —NR^{Xa}R^{Xb}, —C₃-C₈ cycloalkyl;
R^{Xa} and R^{Xb} are independently —H, or —C₁-C₃ alkyl;
each R³ is independently halo, —CN, or —C₁-C₃ haloalkoxy; and
R^{4a} and R^{4b} taken together with the N atom to which they are attached form a 4- to 8-membered heterocycloalkyl having 0-1 additional ring heteroatom selected from N, O, and S; wherein said 4- to 8-membered heterocycloalkyl is substituted with 1-3 halo.

In some embodiments, the compound, or pharmaceutically acceptable salt thereof, has a structure according to Formula Ie:

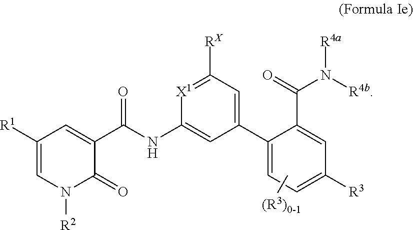

wherein:
R¹ is selected from the group consisting of -(Q¹)-NR^{1a}R^{1b}, and -(Q¹)-(4- to 8-membered heterocycloalkyl) having 1-3 ring heteroatoms independently selected from N, O, and S; and wherein said 4- to 8-membered heterocycloalkyl is substituted with 1-2 —C₁-C₃ alkyl;
Q¹ is unsubstituted —(C₁-C₃ alkylene)-;
R^{1a} and R^{1b} are independently selected from the group consisting of —H, —C₁-C₆ alkyl, —C₁-C₆ haloalkyl —C₃-C₆ cycloalkyl, —(C₁-C₄ alkylene)-O—(C₁-C₃ alkyl), —(C₁-C₃ alkylene)-(C₃-C₆ cycloalkyl), and —(C₁-C₃ alkylene)-(4- to 8-membered heterocycloalkyl) wherein said 4- to 8-membered heterocycloalkyl has 1-2 ring heteroatoms independently selected from N and O, wherein said —C₃-C₆ cycloalkyl, and —(C₁-C₃ alkylene)-(C₃-C₆ cycloalkyl), are unsubstituted or substituted with 1-3 R^{1c};
each R^{1c}, when present, is independently halo, —OH, —C₁-C₃ alkyl, or —C₁-C₃ alkoxy;
R² is —C₁-C₆ alkyl, —C₁-C₆ haloalkyl, or —C₃-C₈ cycloalkyl;
X¹ is N or CH;
R^{X} is halo, —CN, —C₁-C₆ alkyl, —C₁-C₆ alkoxy, —C₁-C₆ haloalkoxy, —NR^{Xa}R^{Xb}, or —C₃-C₈ cycloalkyl;
R^{Xa} and R^{Xb} are independently —H, or —C₁-C₃ alkyl;
each R³ is independently halo, —CN, —C₁-C₃ alkyl, —C₁-C₃ haloalkyl, —C₁-C₃ alkoxy, or —C₁-C₃ haloalkoxy; and
R^{4a} and R^{4b} are independently H or —C₁-C₆ alkyl; or
R^{4a} and R^{4b} taken together with the N atom to which they are attached form a 4- to 8-membered heterocycloalkyl having 0-1 additional ring heteroatom selected from N, O, and S; wherein said 4- to 8-membered heterocycloalkyl is unsubstituted or substituted with 1-3 substituents independently selected from the group consisting of halo, —CN, and —C₁-C₃ alkyl.

In some embodiments, R¹ is —(C₁-C₂ alkylene)-NR^{1a}R^{1b}, or —(C₁-C₂ alkylene)-(4- to 8-membered heterocycloalkyl), wherein said 4- to 8-membered heterocycloalkyl has 1-2 ring N atoms, and is unsubstituted or substituted with —C₁-C₃ alkyl; R^{1a} and R^{1b} are independently selected from the group consisting of —H, —C₁-C₆ alkyl, —C₁-C₆ haloalkyl, —(C₁-C₄ alkylene)-O—(C₁-C₃ alkyl), —C₃-C₆ cycloalkyl, —(C₁-C₃ alkylene)-(C₃-C₆ cycloalkyl), and —(C₁-C₃ alkylene)-(4- to 8-membered heterocycloalkyl) wherein said 4- to 8-membered heterocycloalkyl has 1-2 ring heteroatoms independently selected from N and O; wherein said —(C₁-C₃ alkylene)-O—(C₁-C₃ alkyl), —C₃-C₆ cycloalkyl, —(C₁-C₃ alkylene)-(C₃-C₆ cycloalkyl), 4- to 8-membered heterocycloalkyl, and —(C₁-C₃ alkylene)-(4- to 8-membered heterocycloalkyl) are unsubstituted or substituted with 1-3 R^{1c}; and
each R^{1c}, when present, is independently halo, —OH, —C₁-C₃ alkyl, or —C₁-C₃ alkoxyl.

In some embodiments, R¹ is

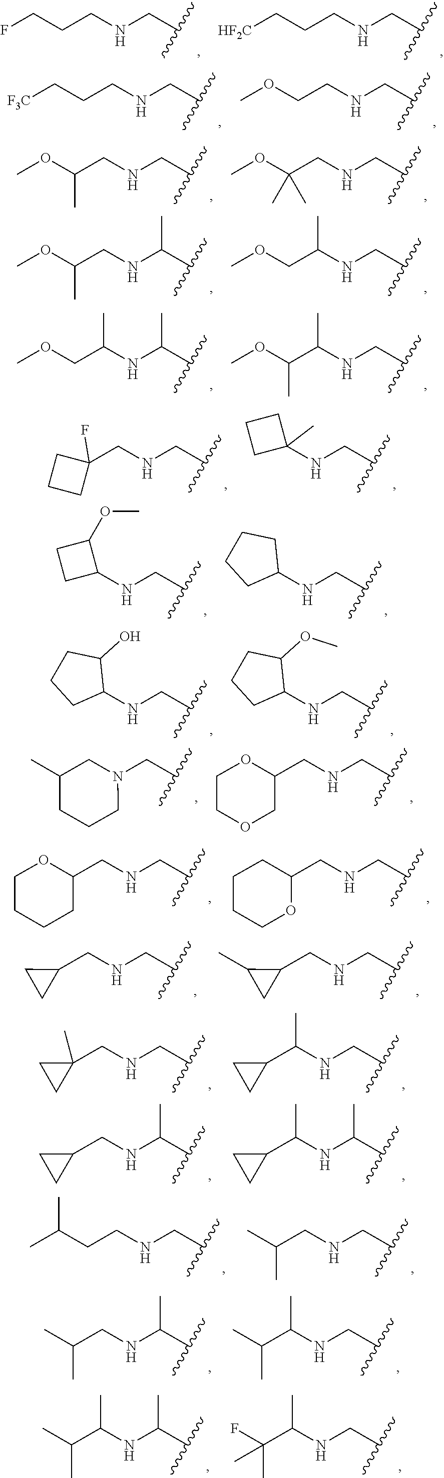

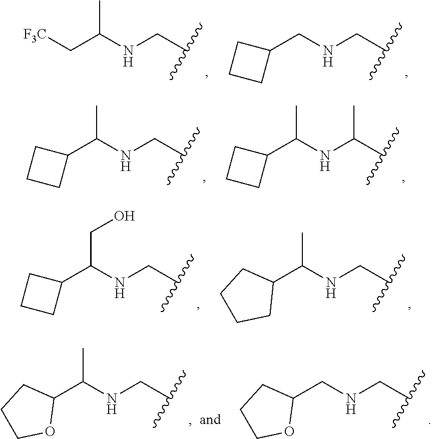

In some embodiments, R² is —C₃-C₆ cycloalkyl, C₁-C₃ alkyl, or C₁-C₃ haloalkyl. In some embodiments R² is

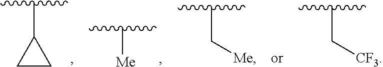

In some embodiments R^{X} is halo, —CN, —C₁-C₃ alkyl, —C₁-C₃ alkoxy, —C₁-C₃ haloalkoxy, —NR^{Xa}R^{Xb}, or —C₃-C₆ cycloalkyl. In some embodiments, R^{X} is —Cl, —CN, —CH₃, —OCH₂, —OCH₂CH₃, —OCH(CH₃)₂, —OCF₂H, —OCH₂CF₂H, —OCH₂CF₃, NR^{Xa}R^{Xb}, or cyclopropyl; wherein R^{Xa} and R^{Xb} are independently —H, —CH₃, —CH₂CH₃, or —CH(CH₃)₂.

In some embodiments, each R³ is independently —Cl, —F, —CN, —CF₂H, —OCH₃, or —OCF₂H.

In some embodiments, R^{4a} and R^{4b} are independently H or —C₁-C₃ alkyl. In some embodiments, R^{4a} and R^{4b} are independently C₁-C₃ alkyl. In some embodiments, R^{4a} and R^{4b} are indpependently —CH₃.

In some embodiments, R^{4a} and R^{4b} are taken together with the N atom to which they are attached to form a 4- to 8-membered heterocycloalkyl having 0-1 additional ring heteroatom selected from N, O, and S; wherein said 4- to 8-membered heterocycloalkyl is unsubstituted or substituted with 1-2 substituents independently selected from halo, —CN, and —C₁-C₆ alkyl. In some embodiments, R⁴ is

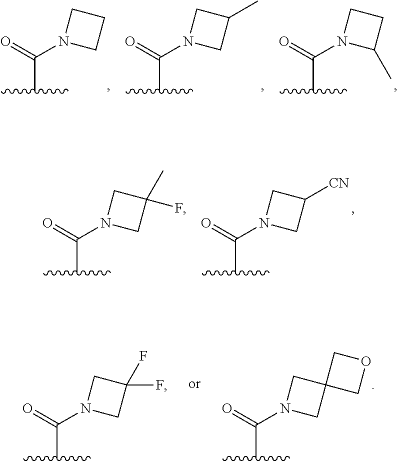

In some embodiments R^{4a} and R^{4b} are taken together with the N atom to which they are attached to form an azetidine ring, wherein said azetidine ring is unsubstituted or substituted with 1-2 substituents independently selected from halo, —CN, and —C₁-C₆ alkyl. In some embodiments, R⁴ is

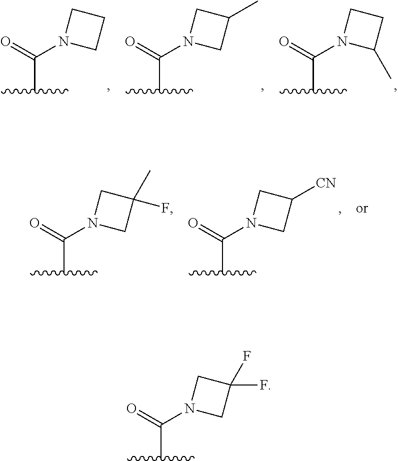

In some embodiments, the compound, or pharmaceutically acceptable salt thereof, has a structure according to Formula Ig:

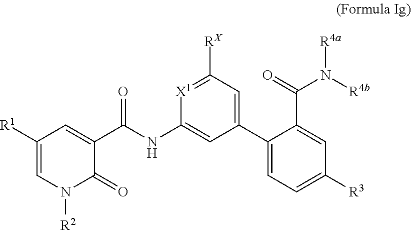

wherein:
R¹ is selected from the group consisting of -(Q¹)-NR^{1a}R^{1b} and -(Q¹)-(4- to 8-membered heterocycloalkyl) having 1-3 ring heteroatoms independently selected from N, O, and S; and wherein said 4- to 8-membered heterocycloalkyl is substituted with 1-2 —C₁-C₃ alkyl;
Q¹ is unsubstituted —(C₁-C₃ alkylene)-;
R^{1a} and R^{1b} are independently selected from the group consisting of —H, —C₁-C₆ alkyl, —(C₁-C₃ alkylene)-O—(C₁-C₃ alkyl), —(C₁-C₃ alkylene)-(C₃-C₆ cycloalkyl), and —(C₁-C₃ alkylene)-(4- to 8-membered heterocycloalkyl) wherein said 4- to 8-membered heterocycloalkyl has 1-2 ring heteroatoms independently selected from N and O;
R² is —C₁-C₆ alkyl, —C₁-C₆ haloalkyl, or —C₃-C₈ cycloalkyl;
X¹ is N or CH;
R^{X} is —CN, —C₁-C₆ alkoxy, —C₁-C₆ haloalkoxy, —NR^{Xa}R^{Xb}, —C₃-C₈ cycloalkyl;
R^{Xa} and R^{Xb} are independently —H, or —C₁-C₃ alkyl;
R³ is halo, —CN, or —C₁-C₃ haloalkoxy; and
R^{4a} and R^{4b} taken together with the N atom to which they are attached form a 4- to 8-membered heterocycloalkyl having 0-1 additional ring heteroatom selected from N, O, and S; wherein said 4- to 8-membered heterocycloalkyl is substituted with 1-3 halo.

In some embodiments, the compound, or pharmaceutically acceptable salt thereof, has a structure according to Formula Ig:

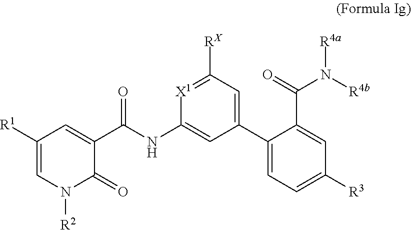

wherein:
R¹ is selected from the group consisting of -(Q¹)-NR^{1a}R^{1b}, and -(Q¹)-(4- to 8-membered heterocycloalkyl) having 1-3 ring heteroatoms independently selected from N, O, and S; and wherein said 4- to 8-membered heterocycloalkyl is substituted with 1-2 —C₁-C₃ alkyl;
Q¹ is unsubstituted —(C₁-C₃ alkylene)-;
R^{1a} and R^{1b} are independently selected from the group consisting of —H, and —(C₁-C₃ alkylene)-O—(C₁-C₃ alkyl);
R² is —C₃-C₈ cycloalkyl;
X¹ is N or CH;
R^{X} is —CN or —C₃-C₈ cycloalkyl;
R³ is halo; and
R^{4a} and R^{4b} taken together with the N atom to which they are attached form a 4- to 8-membered heterocycloalkyl having 0-1 additional ring heteroatom selected from N, O, and S; wherein said 4- to 8-membered heterocycloalkyl is substituted with 1-3 halo.

In one or more embodiments, the compound, or pharmaceutically acceptable salt or solvate thereof, according to this disclosure is selected from the compounds provided in Table 1, Table 2, Table 3, Table 4 or Table 5. In some embodiments, the compound is selected from the compounds provided in Table 1, Table 2, Table 3, Table 4, or Table 5.

TABLE 1

| Example | Structure |
|---|---|
| 1 | 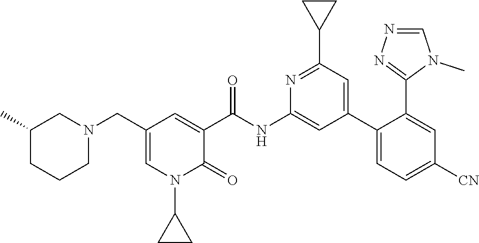 |
| 2 | 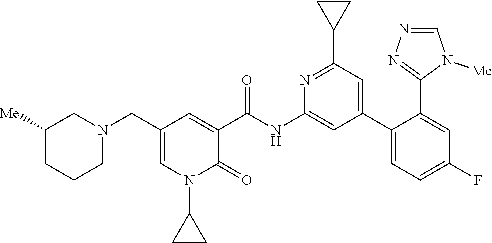 [SMILES: C[C@H]1CCCN(Cc2cc(C(=O)Nc3cc(-c4ccc(F)cc4-c4nncn4C)cc(C4CC4)n3)c(=O)n(C3CC3)c2)C1] |
| 3 | 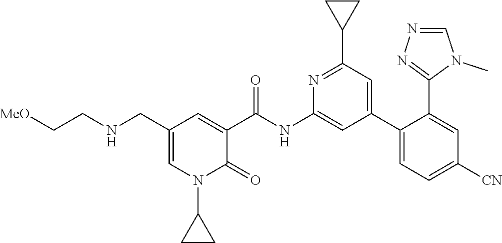 [SMILES: COCCNCc1cc(C(=O)Nc2cc(-c3ccc(C#N)cc3-c3nncn3C)cc(C3CC3)n2)c(=O)n(C2CC2)c1] |
| 4 | 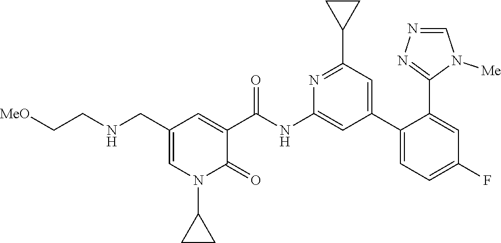 [SMILES: COCCNCc1cc(C(=O)Nc2cc(-c3ccc(F)cc3-c3nncn3C)cc(C3CC3)n2)c(=O)n(C2CC2)c1] |
| 5 | 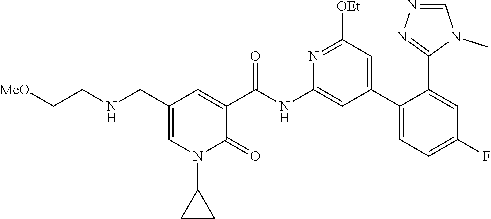 [SMILES: CCOc1cc(-c2ccc(F)cc2-c2nncn2C)cc(NC(=O)c2cc(CNCCOC)cn(C3CC3)c2=O)n1] |
| 6 | 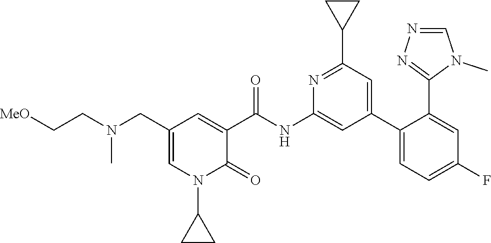 [SMILES: COCCN(C)Cc1cc(C(=O)Nc2cc(-c3ccc(F)cc3-c3nncn3C)cc(C3CC3)n2)c(=O)n(C2CC2)c1] |
| 7 | 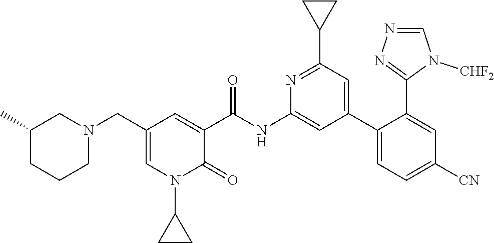 [SMILES: C[C@H]1CCCN(Cc2cc(C(=O)Nc3cc(-c4ccc(C#N)cc4-c4nncn4C(F)F)cc(C4CC4)n3)c(=O)n(C3CC3)c2)C1] |
| 8 | 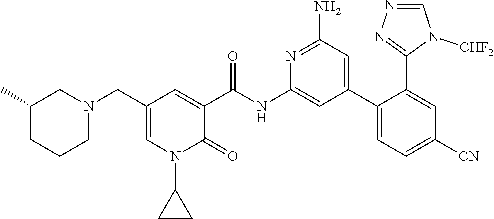 [SMILES: C[C@H]1CCCN(Cc2cc(C(=O)Nc3cc(-c4ccc(C#N)cc4-c4nncn4C(F)F)cc(N)n3)c(=O)n(C3CC3)c2)C1] |
| 9 | 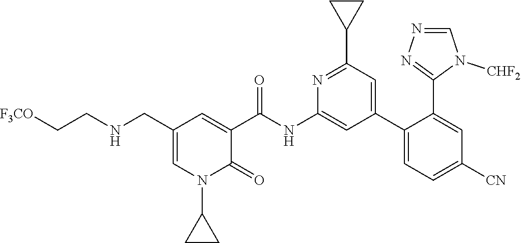 [SMILES: N#Cc1ccc(-c2cc(NC(=O)c3cc(CNCCOC(F)(F)F)cn(C4CC4)c3=O)nc(C3CC3)c2)c(-c2nncn2C(F)F)c1] |
| 10 | 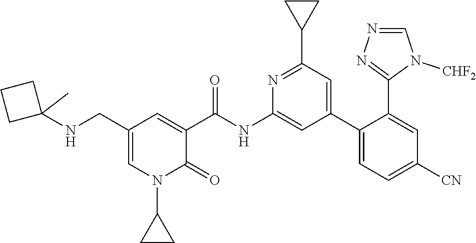 [SMILES: CC1(NCc2cc(C(=O)Nc3cc(-c4ccc(C#N)cc4-c4nncn4C(F)F)cc(C4CC4)n3)c(=O)n(C3CC3)c2)CCC1] |
| 11 | 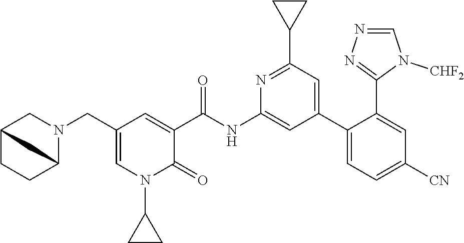 [SMILES: N#Cc1ccc(-c2cc(NC(=O)c3cc(CN4C[C@H]5CC[C@@H]4C5)cn(C4CC4)c3=O)nc(C3CC3)c2)c(-c2nncn2C(F)F)c1] |
| 12 | 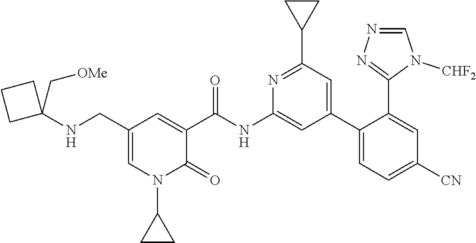 [SMILES: COCC1(NCc2cc(C(=O)Nc3cc(-c4ccc(C#N)cc4-c4nncn4C(F)F)cc(C4CC4)n3)c(=O)n(C3CC3)c2)CCC1] |
| 13 | 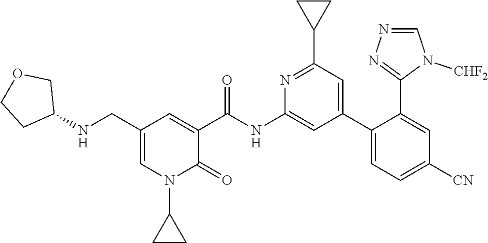 [SMILES: N#Cc1ccc(-c2cc(NC(=O)c3cc(CN[C@@H]4CCOC4)cn(C4CC4)c3=O)nc(C3CC3)c2)c(-c2nncn2C(F)F)c1] |
| 14 | 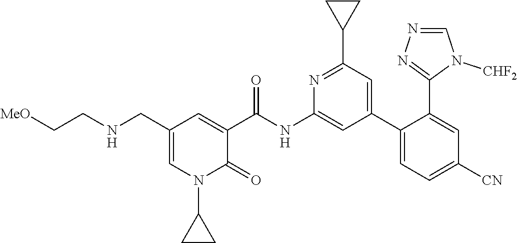 [SMILES: COCCNCc1cc(C(=O)Nc2cc(-c3ccc(C#N)cc3-c3nncn3C(F)F)cc(C3CC3)n2)c(=O)n(C2CC2)c1] |
| 15 | 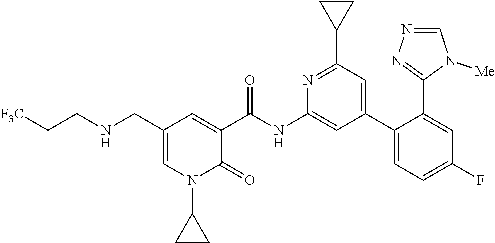 [SMILES: Cn1cnnc1-c1cc(F)ccc1-c1cc(NC(=O)c2cc(CNCCC(F)(F)F)cn(C3CC3)c2=O)nc(C2CC2)c1] |
| 16 | 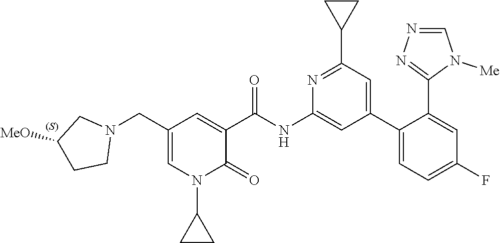 |
| 17 | 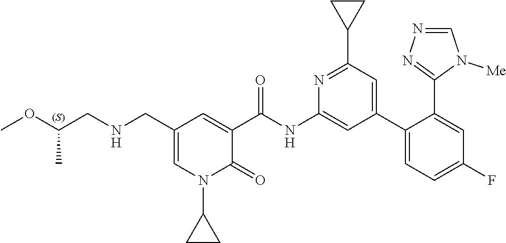 |
| 18 | 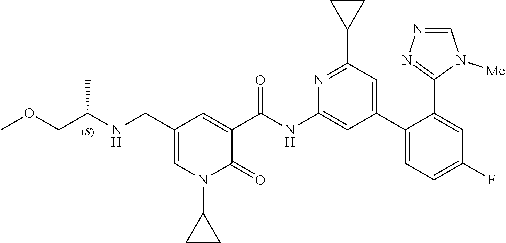 |
| 19 | 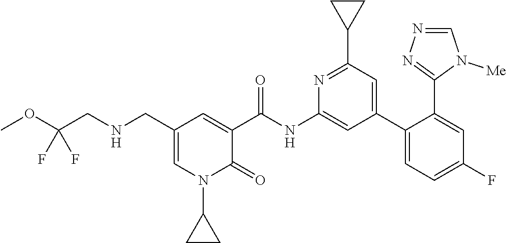 [SMILES: COC(F)(F)CNCc1cc(C(=O)Nc2cc(-c3ccc(F)cc3-c3nncn3C)cc(C3CC3)n2)c(=O)n(C2CC2)c1] |
| 20 | 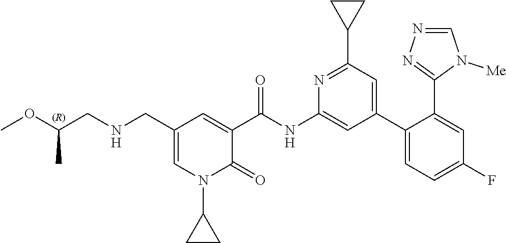 |
| 21 | 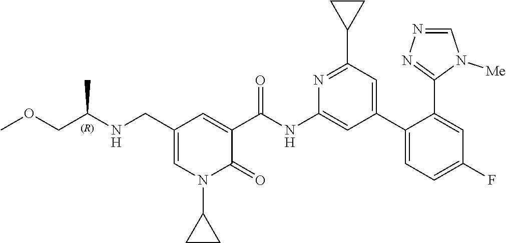 |
| 22 | 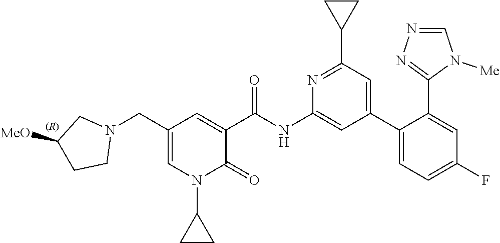 |
| 23 | 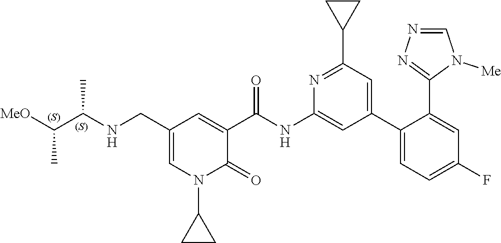 |
| 24 | 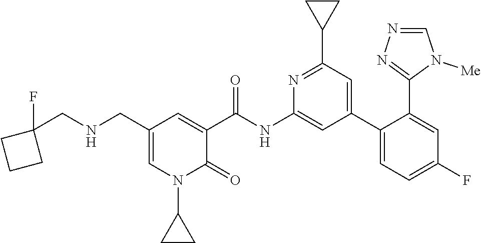 [SMILES: Cn1cnnc1-c1cc(F)ccc1-c1cc(NC(=O)c2cc(CNCC3(F)CCC3)cn(C3CC3)c2=O)nc(C2CC2)c1] |
| 25 | 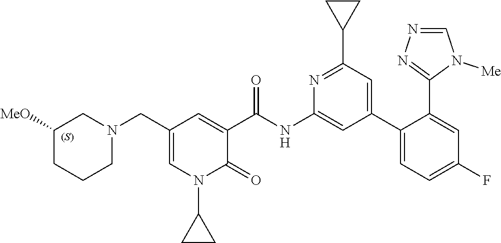 |
| 26 | 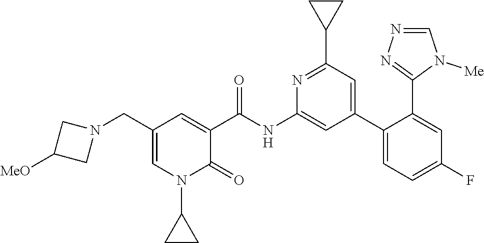 [SMILES: COC1CN(Cc2cc(C(=O)Nc3cc(-c4ccc(F)cc4-c4nncn4C)cc(C4CC4)n3)c(=O)n(C3CC3)c2)C1] |
| 27 | 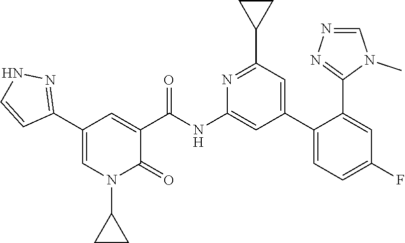 [SMILES: Cn1cnnc1-c1cc(F)ccc1-c1cc(NC(=O)c2cc(-c3cc[nH]n3)cn(C3CC3)c2=O)nc(C2CC2)c1] |
| 28 | 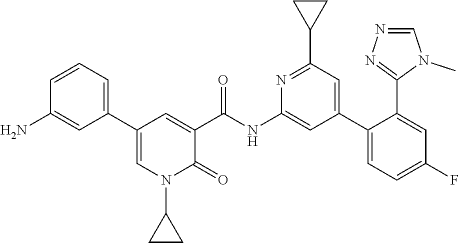 [SMILES: Cn1cnnc1-c1cc(F)ccc1-c1cc(NC(=O)c2cc(-c3cccc(N)c3)cn(C3CC3)c2=O)nc(C2CC2)c1] |
| 29 | 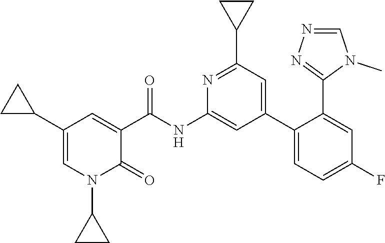 [SMILES: Cn1cnnc1-c1cc(F)ccc1-c1cc(NC(=O)c2cc(C3CC3)cn(C3CC3)c2=O)nc(C2CC2)c1] |
| 30 | 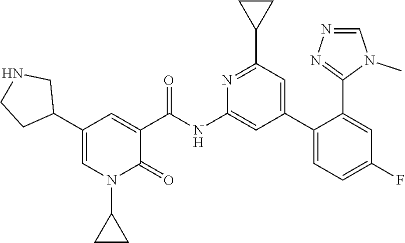 [SMILES: Cn1cnnc1-c1cc(F)ccc1-c1cc(NC(=O)c2cc(C3CCNC3)cn(C3CC3)c2=O)nc(C2CC2)c1] |
| 31 | 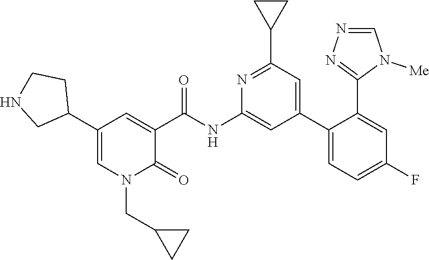 [SMILES: Cn1cnnc1-c1cc(F)ccc1-c1cc(NC(=O)c2cc(C3CCNC3)cn(CC3CC3)c2=O)nc(C2CC2)c1] |
| 32 | 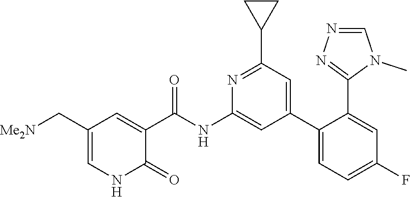 [SMILES: CN(C)Cc1c[nH]c(=O)c(C(=O)Nc2cc(-c3ccc(F)cc3-c3nncn3C)cc(C3CC3)n2)c1] |
| 33 | 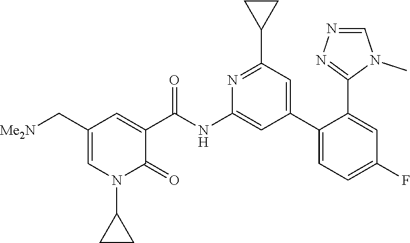 [SMILES: CN(C)Cc1cc(C(=O)Nc2cc(-c3ccc(F)cc3-c3nncn3C)cc(C3CC3)n2)c(=O)n(C2CC2)c1] |
| 34 | 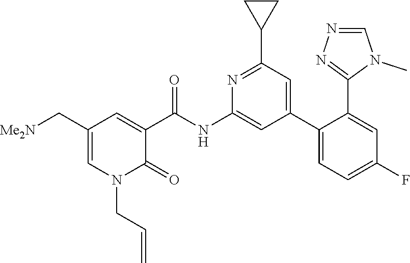 [SMILES: C=CCn1cc(CN(C)C)cc(C(=O)Nc2cc(-c3ccc(F)cc3-c3nncn3C)cc(C3CC3)n2)c1=O] |
| 35 | 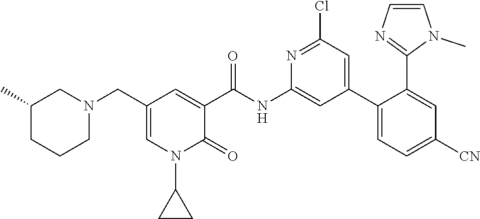 |
| 36 | 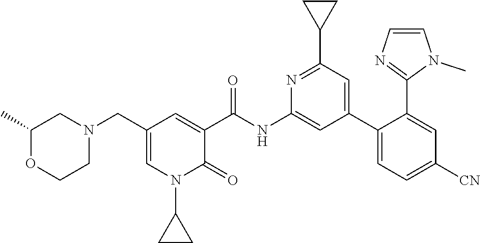 |
| 37 | 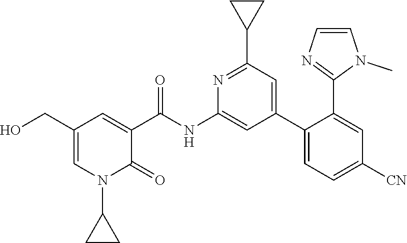 [SMILES: Cn1ccnc1-c1cc(C#N)ccc1-c1cc(NC(=O)c2cc(CO)cn(C3CC3)c2=O)nc(C2CC2)c1] |
| 38 | 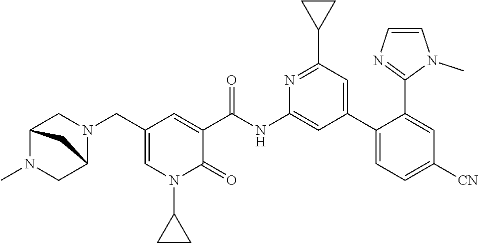 |
| 39 | 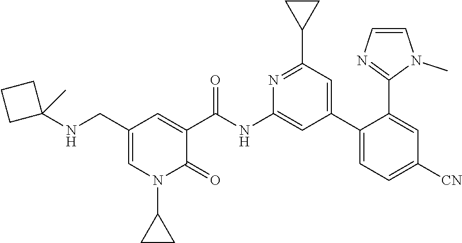 [SMILES: Cn1ccnc1-c1cc(C#N)ccc1-c1cc(NC(=O)c2cc(CNC3(C)CCC3)cn(C3CC3)c2=O)nc(C2CC2)c1] |
| 40 | 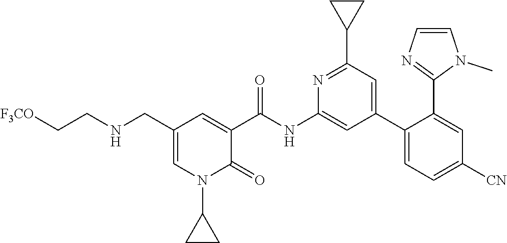 [SMILES: Cn1ccnc1-c1cc(C#N)ccc1-c1cc(NC(=O)c2cc(CNCCOC(F)(F)F)cn(C3CC3)c2=O)nc(C2CC2)c1] |
| 41 | 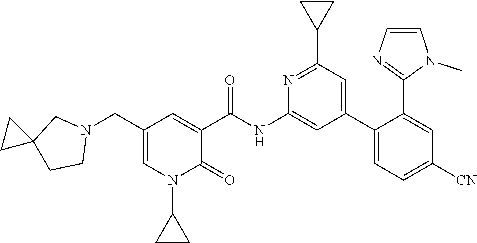 [SMILES: Cn1ccnc1-c1cc(C#N)ccc1-c1cc(NC(=O)c2cc(CN3CCC4(CC4)C3)cn(C3CC3)c2=O)nc(C2CC2)c1] |
| 42 | 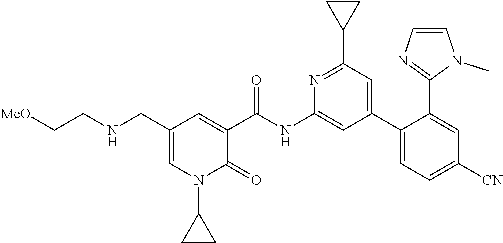 [SMILES: COCCNCc1cc(C(=O)Nc2cc(-c3ccc(C#N)cc3-c3nccn3C)cc(C3CC3)n2)c(=O)n(C2CC2)c1] |
| 43 | 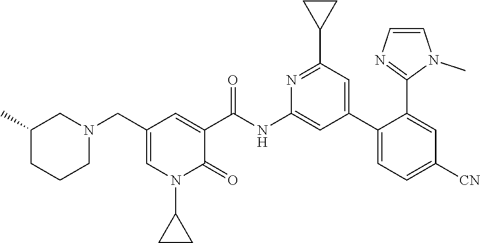 |
| 44 | 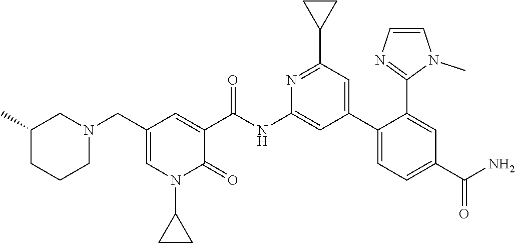 [SMILES: C[C@H]1CCCN(Cc2cc(C(=O)Nc3cc(-c4ccc(C(N)=O)cc4-c4nccn4C)cc(C4CC4)n3)c(=O)n(C3CC3)c2)C1] |
| 45 | 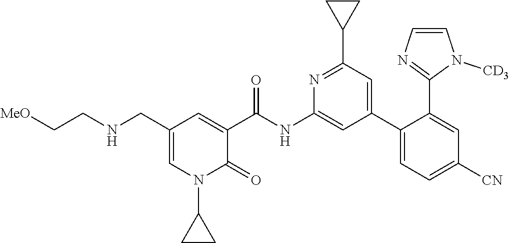 [SMILES: [2H]C([2H])([2H])n1ccnc1-c1cc(C#N)ccc1-c1cc(NC(=O)c2cc(CNCCOC)cn(C3CC3)c2=O)nc(C2CC2)c1] |
| 46 | 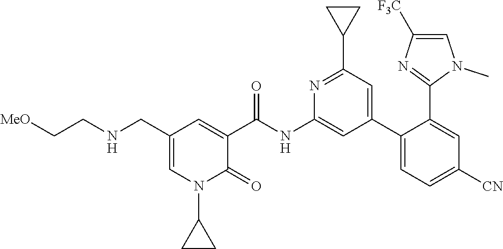 [SMILES: COCCNCc1cc(C(=O)Nc2cc(-c3ccc(C#N)cc3-c3nc(C(F)(F)F)cn3C)cc(C3CC3)n2)c(=O)n(C2CC2)c1] |
| 47 | 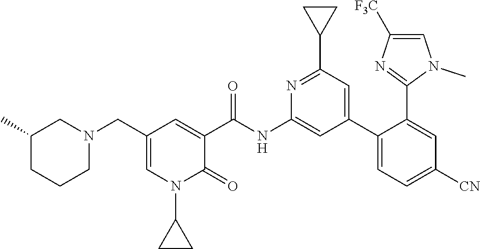 |
| 48 | 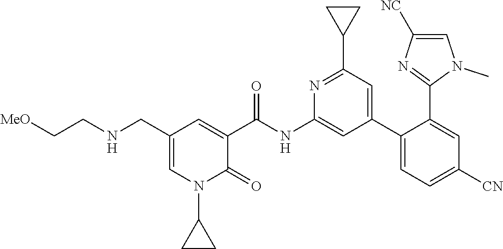 [SMILES: COCCNCc1cc(C(=O)Nc2cc(-c3ccc(C#N)cc3-c3nc(C#N)cn3C)cc(C3CC3)n2)c(=O)n(C2CC2)c1] |
| 49 | 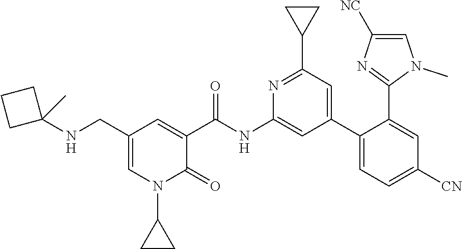 [SMILES: Cn1cc(C#N)nc1-c1cc(C#N)ccc1-c1cc(NC(=O)c2cc(CNC3(C)CCC3)cn(C3CC3)c2=O)nc(C2CC2)c1] |
| 50 | 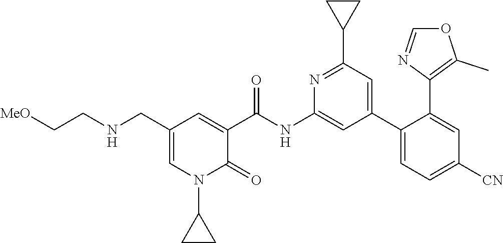 [SMILES: COCCNCc1cc(C(=O)Nc2cc(-c3ccc(C#N)cc3-c3ncoc3C)cc(C3CC3)n2)c(=O)n(C2CC2)c1] |
| 51 | 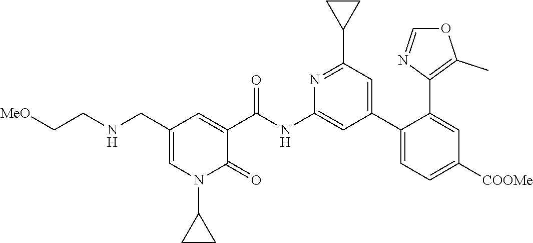 [SMILES: COCCNCc1cc(C(=O)Nc2cc(-c3ccc(C(=O)OC)cc3-c3ncoc3C)cc(C3CC3)n2)c(=O)n(C2CC2)c1] |
| 52 | 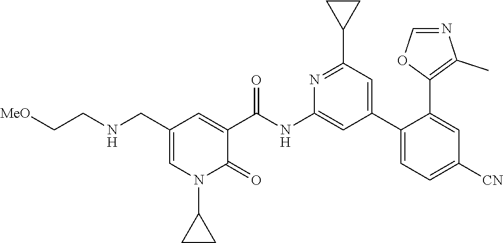 [SMILES: COCCNCc1cc(C(=O)Nc2cc(-c3ccc(C#N)cc3-c3ocnc3C)cc(C3CC3)n2)c(=O)n(C2CC2)c1] |
| 53 | 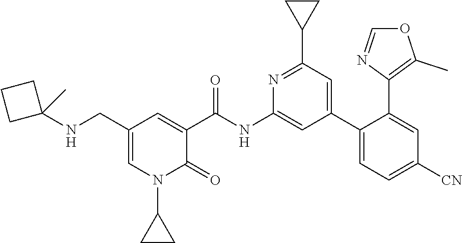 [SMILES: Cc1ocnc1-c1cc(C#N)ccc1-c1cc(NC(=O)c2cc(CNC3(C)CCC3)cn(C3CC3)c2=O)nc(C2CC2)c1] |
| 54 | 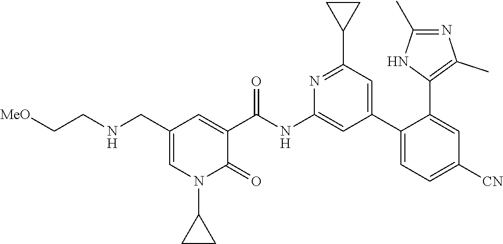 [SMILES: COCCNCc1cc(C(=O)Nc2cc(-c3ccc(C#N)cc3-c3[nH]c(C)nc3C)cc(C3CC3)n2)c(=O)n(C2CC2)c1] |
| 55 | 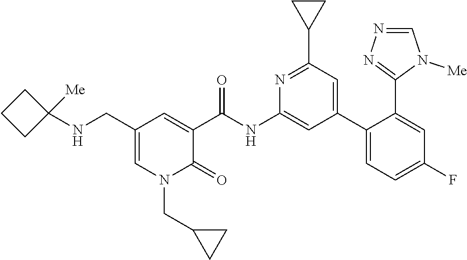 [SMILES: Cn1cnnc1-c1cc(F)ccc1-c1cc(NC(=O)c2cc(CNC3(C)CCC3)cn(CC3CC3)c2=O)nc(C2CC2)c1] |
| 56 | 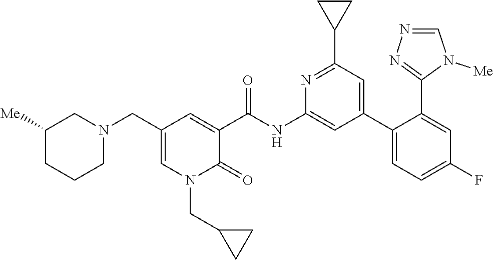 [SMILES: C[C@H]1CCCN(Cc2cc(C(=O)Nc3cc(-c4ccc(F)cc4-c4nncn4C)cc(C4CC4)n3)c(=O)n(CC3CC3)c2)C1] |
| 57 | 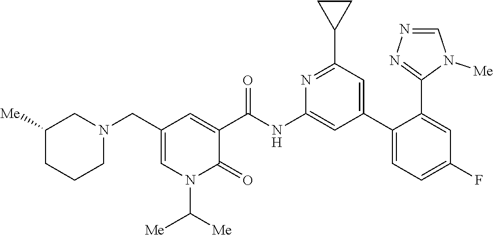 [SMILES: CC(C)n1cc(CN2CCC[C@H](C)C2)cc(C(=O)Nc2cc(-c3ccc(F)cc3-c3nncn3C)cc(C3CC3)n2)c1=O] |
| 58 | 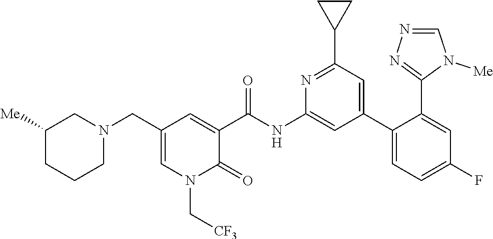 [SMILES: C[C@H]1CCCN(Cc2cc(C(=O)Nc3cc(-c4ccc(F)cc4-c4nncn4C)cc(C4CC4)n3)c(=O)n(CC(F)(F)F)c2)C1] |
| 59 | 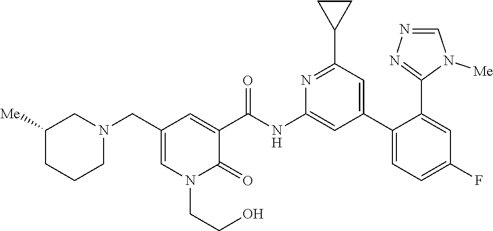 [SMILES: C[C@H]1CCCN(Cc2cc(C(=O)Nc3cc(-c4ccc(F)cc4-c4nncn4C)cc(C4CC4)n3)c(=O)n(CCO)c2)C1] |
| 60 | 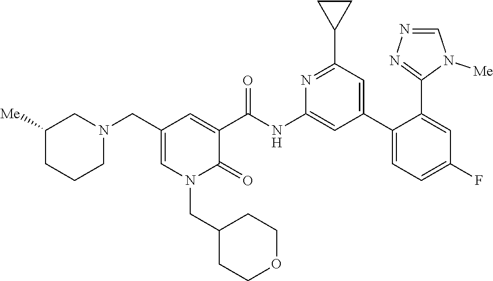 [SMILES: C[C@H]1CCCN(Cc2cc(C(=O)Nc3cc(-c4ccc(F)cc4-c4nncn4C)cc(C4CC4)n3)c(=O)n(CC3CCOCC3)c2)C1] |
| 61 | 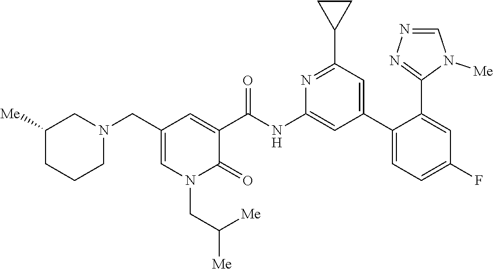 [SMILES: CC(C)Cn1cc(CN2CCC[C@H](C)C2)cc(C(=O)Nc2cc(-c3ccc(F)cc3-c3nncn3C)cc(C3CC3)n2)c1=O] |
| 62 | 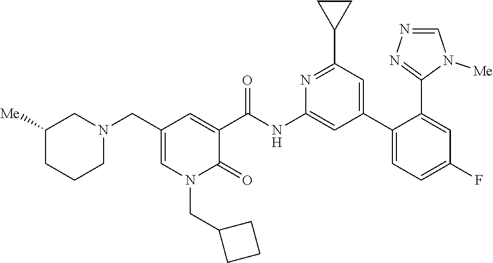 [SMILES: C[C@H]1CCCN(Cc2cc(C(=O)Nc3cc(-c4ccc(F)cc4-c4nncn4C)cc(C4CC4)n3)c(=O)n(CC3CCC3)c2)C1] |
| 63 | 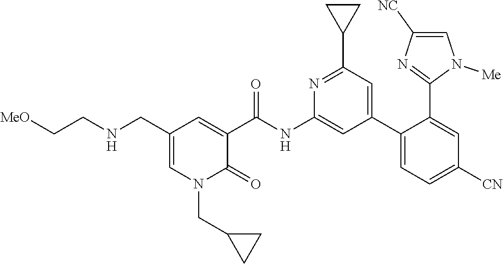 [SMILES: COCCNCc1cc(C(=O)Nc2cc(-c3ccc(C#N)cc3-c3nc(C#N)cn3C)cc(C3CC3)n2)c(=O)n(CC2CC2)c1] |
| 64 | 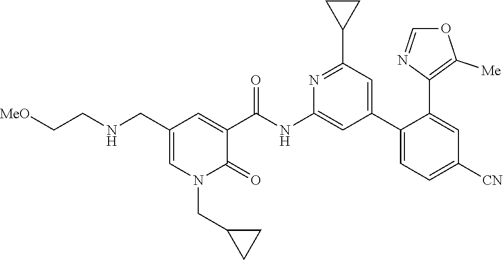 [SMILES: COCCNCc1cc(C(=O)Nc2cc(-c3ccc(C#N)cc3-c3ncoc3C)cc(C3CC3)n2)c(=O)n(CC2CC2)c1] |
| 65 | 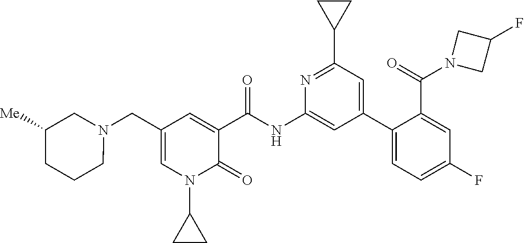 [SMILES: C[C@H]1CCCN(Cc2cc(C(=O)Nc3cc(-c4ccc(F)cc4C(=O)N4CC(F)C4)cc(C4CC4)n3)c(=O)n(C3CC3)c2)C1] |
| 66 | 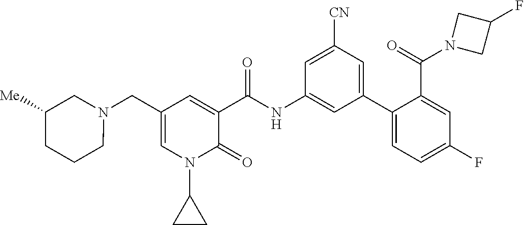 [SMILES: C[C@H]1CCCN(Cc2cc(C(=O)Nc3cc(C#N)cc(-c4ccc(F)cc4C(=O)N4CC(F)C4)c3)c(=O)n(C3CC3)c2)C1] |
| 67 | 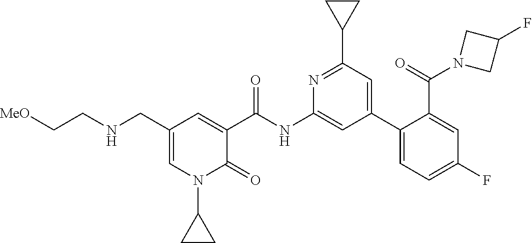 [SMILES: COCCNCc1cc(C(=O)Nc2cc(-c3ccc(F)cc3C(=O)N3CC(F)C3)cc(C3CC3)n2)c(=O)n(C2CC2)c1] |
| 68 | 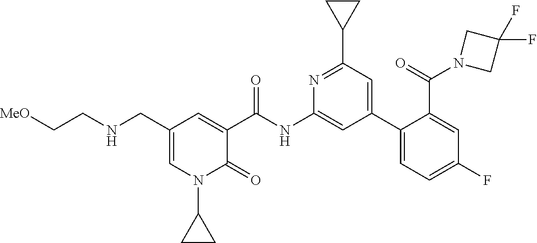 [SMILES: COCCNCc1cc(C(=O)Nc2cc(-c3ccc(F)cc3C(=O)N3CC(F)(F)C3)cc(C3CC3)n2)c(=O)n(C2CC2)c1] |
| 69 | 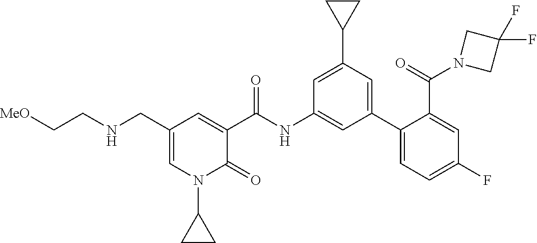 [SMILES: COCCNCc1cc(C(=O)Nc2cc(-c3ccc(F)cc3C(=O)N3CC(F)(F)C3)cc(C3CC3)c2)c(=O)n(C2CC2)c1] |
| 70 | 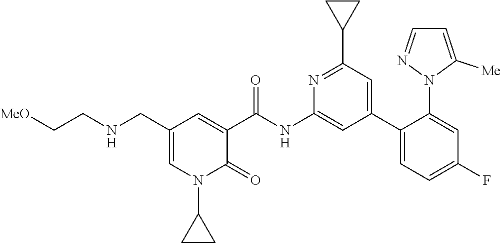 [SMILES: COCCNCc1cc(C(=O)Nc2cc(-c3ccc(F)cc3-n3nccc3C)cc(C3CC3)n2)c(=O)n(C2CC2)c1] |
| 71 | 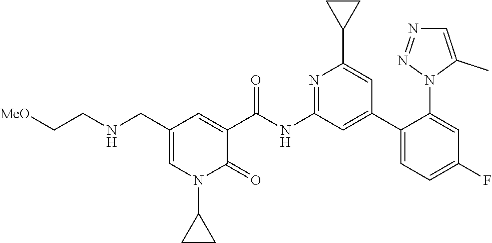 [SMILES: COCCNCc1cc(C(=O)Nc2cc(-c3ccc(F)cc3-n3nncc3C)cc(C3CC3)n2)c(=O)n(C2CC2)c1] |
| 72 | 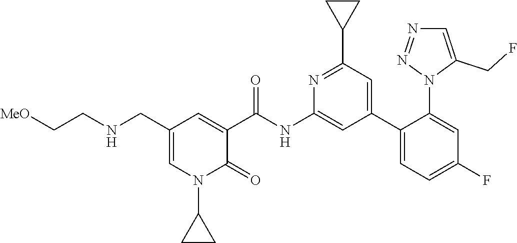 [SMILES: COCCNCc1cc(C(=O)Nc2cc(-c3ccc(F)cc3-n3nncc3CF)cc(C3CC3)n2)c(=O)n(C2CC2)c1] |
| 73 | 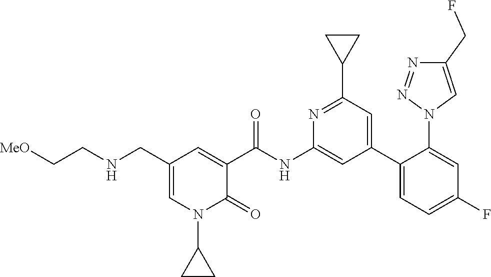 [SMILES: COCCNCc1cc(C(=O)Nc2cc(-c3ccc(F)cc3-n3cc(CF)nn3)cc(C3CC3)n2)c(=O)n(C2CC2)c1] |
| 74 | 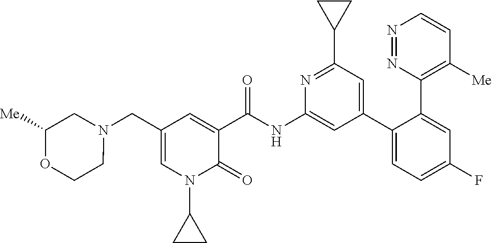 [SMILES: Cc1ccnnc1-c1cc(F)ccc1-c1cc(NC(=O)c2cc(CN3CCO[C@H](C)C3)cn(C3CC3)c2=O)nc(C2CC2)c1] |
| 75 | 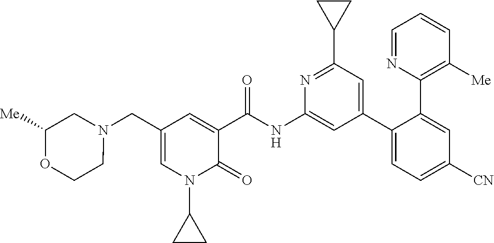 [SMILES: Cc1cccnc1-c1cc(C#N)ccc1-c1cc(NC(=O)c2cc(CN3CCO[C@H](C)C3)cn(C3CC3)c2=O)nc(C2CC2)c1] |
| 76 | 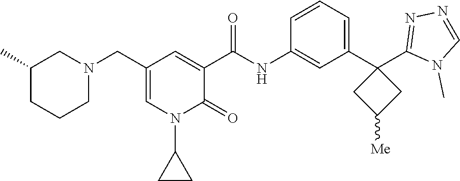 [SMILES: CC1CC(c2cccc(NC(=O)c3cc(CN4CCC[C@H](C)C4)cn(C4CC4)c3=O)c2)(c2nncn2C)C1] |
| 77 | 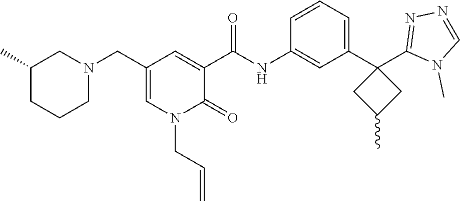 [SMILES: C=CCn1cc(CN2CCC[C@H](C)C2)cc(C(=O)Nc2cccc(C3(c4nncn4C)CC(C)C3)c2)c1=O] |
| 78 | 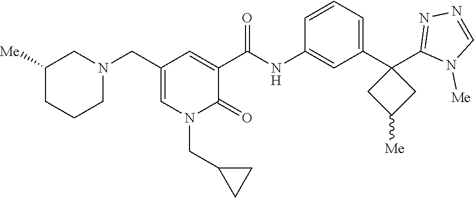 [SMILES: CC1CC(c2cccc(NC(=O)c3cc(CN4CCC[C@H](C)C4)cn(CC4CC4)c3=O)c2)(c2nncn2C)C1] |
| 79 | 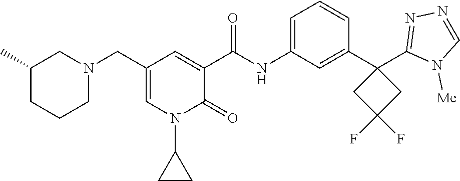 [SMILES: C[C@H]1CCCN(Cc2cc(C(=O)Nc3cccc(C4(c5nncn5C)CC(F)(F)C4)c3)c(=O)n(C3CC3)c2)C1] |
| 80 | 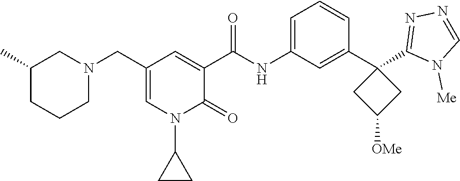 [SMILES: CO[C@H]1C[C@@](c2cccc(NC(=O)c3cc(CN4CCC[C@H](C)C4)cn(C4CC4)c3=O)c2)(c2nncn2C)C1] |
| 81 | 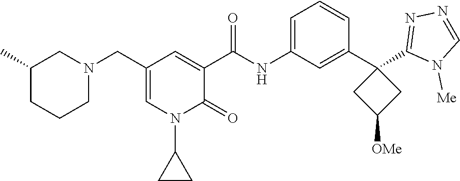 [SMILES: CO[C@H]1C[C@](c2cccc(NC(=O)c3cc(CN4CCC[C@H](C)C4)cn(C4CC4)c3=O)c2)(c2nncn2C)C1] |

TABLE 2

| Example | Structure |
|---|---|
| 82 | 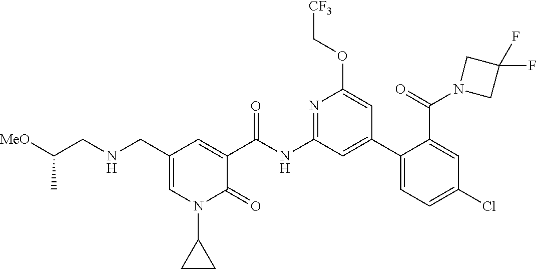 [SMILES: CO[C@@H](C)CNCc1cc(C(=O)Nc2cc(-c3ccc(Cl)cc3C(=O)N3CC(F)(F)C3)cc(OCC(F)(F)F)n2)c(=O)n(C2CC2)c1] |
| 83 | 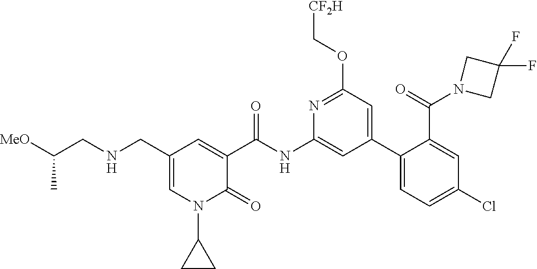 [SMILES: CO[C@@H](C)CNCc1cc(C(=O)Nc2cc(-c3ccc(Cl)cc3C(=O)N3CC(F)(F)C3)cc(OCC(F)F)n2)c(=O)n(C2CC2)c1] |
| 84 | 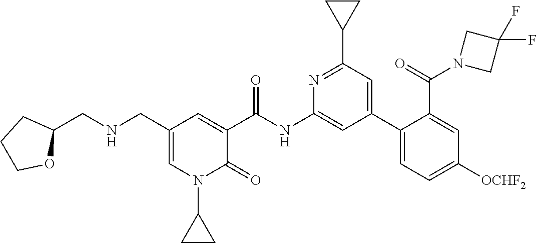 [SMILES: O=C(Nc1cc(-c2ccc(OC(F)F)cc2C(=O)N2CC(F)(F)C2)cc(C2CC2)n1)c1cc(CNC[C@@H]2CCCO2)cn(C2CC2)c1=O] |
| 85 | 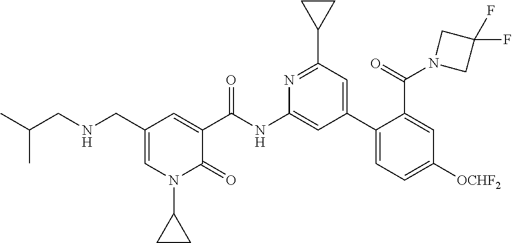 [SMILES: CC(C)CNCc1cc(C(=O)Nc2cc(-c3ccc(OC(F)F)cc3C(=O)N3CC(F)(F)C3)cc(C3CC3)n2)c(=O)n(C2CC2)c1] |
| 86 | 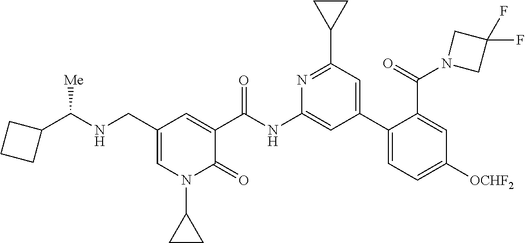 [SMILES: C[C@H](NCc1cc(C(=O)Nc2cc(-c3ccc(OC(F)F)cc3C(=O)N3CC(F)(F)C3)cc(C3CC3)n2)c(=O)n(C2CC2)c1)C1CCC1] |
| 87 | 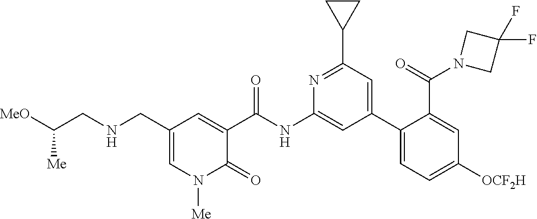 |
| 88 | 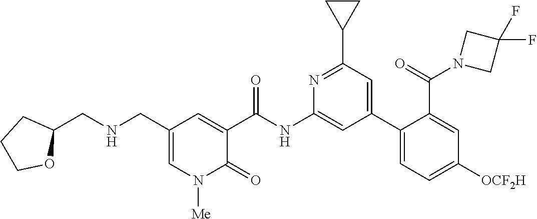 |
| 89 | 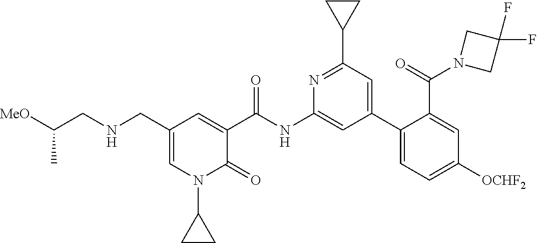 [SMILES: CO[C@@H](C)CNCc1cc(C(=O)Nc2cc(-c3ccc(OC(F)F)cc3C(=O)N3CC(F)(F)C3)cc(C3CC3)n2)c(=O)n(C2CC2)c1] |
| 90 | 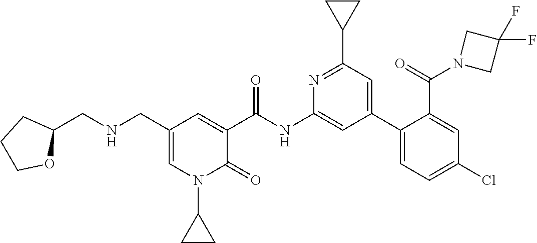 [SMILES: O=C(Nc1cc(-c2ccc(Cl)cc2C(=O)N2CC(F)(F)C2)cc(C2CC2)n1)c1cc(CNC[C@@H]2CCCO2)cn(C2CC2)c1=O] |
| 91 | 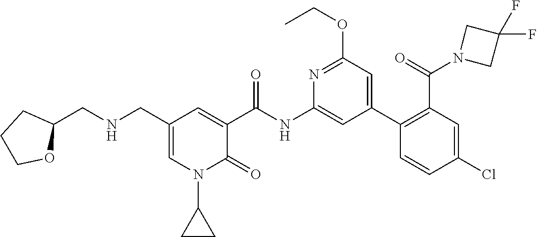 [SMILES: CCOc1cc(-c2ccc(Cl)cc2C(=O)N2CC(F)(F)C2)cc(NC(=O)c2cc(CNC[C@@H]3CCCO3)cn(C3CC3)c2=O)n1] |
| 92 | 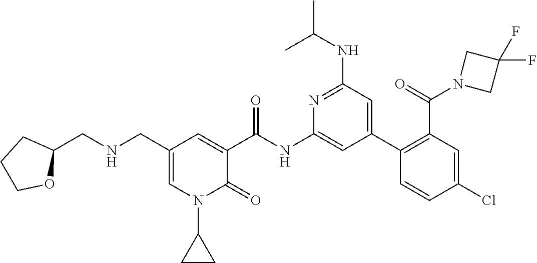 [SMILES: CC(C)Nc1cc(-c2ccc(Cl)cc2C(=O)N2CC(F)(F)C2)cc(NC(=O)c2cc(CNC[C@@H]3CCCO3)cn(C3CC3)c2=O)n1] |
| 93 | 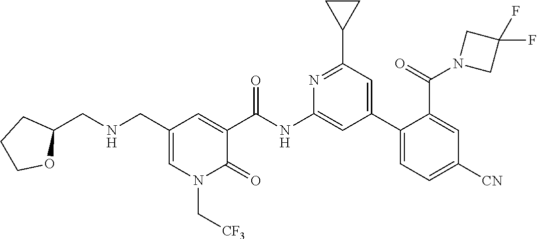 [SMILES: N#Cc1ccc(-c2cc(NC(=O)c3cc(CNC[C@@H]4CCCO4)cn(CC(F)(F)F)c3=O)nc(C3CC3)c2)c(C(=O)N2CC(F)(F)C2)c1] |
| 94 | 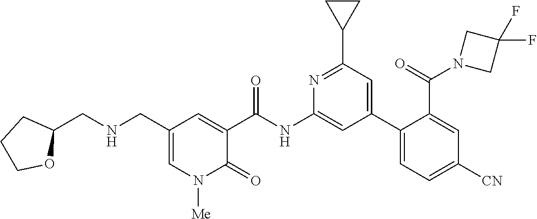 [SMILES: Cn1cc(CNC[C@@H]2CCCO2)cc(C(=O)Nc2cc(-c3ccc(C#N)cc3C(=O)N3CC(F)(F)C3)cc(C3CC3)n2)c1=O] |

TABLE 3

| Example | Structure |
|---|---|
| 95 | 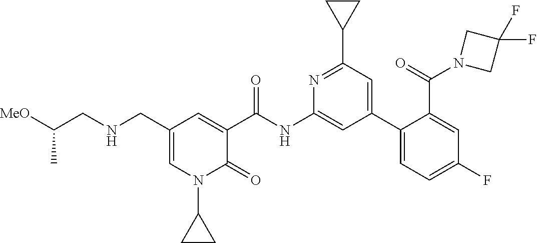 [SMILES: CO[C@@H](C)CNCc1cc(C(=O)Nc2cc(-c3ccc(F)cc3C(=O)N3CC(F)(F)C3)cc(C3CC3)n2)c(=O)n(C2CC2)c1] |
| 96 | 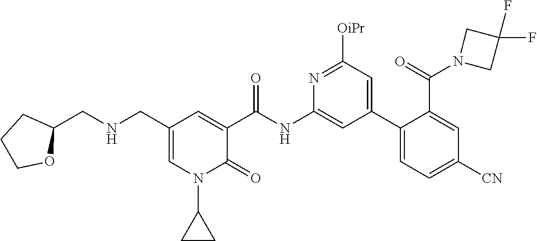 [SMILES: CC(C)Oc1cc(-c2ccc(C#N)cc2C(=O)N2CC(F)(F)C2)cc(NC(=O)c2cc(CNC[C@@H]3CCCO3)cn(C3CC3)c2=O)n1] |
| 97 | 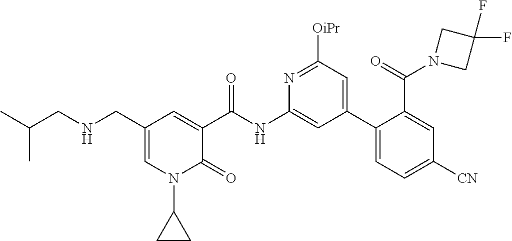 [SMILES: CC(C)CNCc1cc(C(=O)Nc2cc(-c3ccc(C#N)cc3C(=O)N3CC(F)(F)C3)cc(OC(C)C)n2)c(=O)n(C2CC2)c1] |
| 98 | 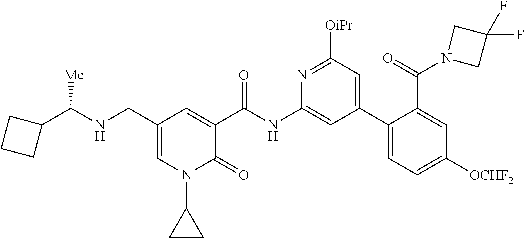 [SMILES: CC(C)Oc1cc(-c2ccc(OC(F)F)cc2C(=O)N2CC(F)(F)C2)cc(NC(=O)c2cc(CN[C@@H](C)C3CCC3)cn(C3CC3)c2=O)n1] |
| 99 | 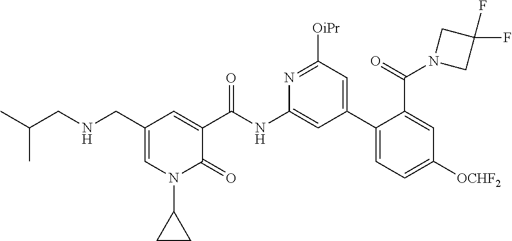 [SMILES: CC(C)CNCc1cc(C(=O)Nc2cc(-c3ccc(OC(F)F)cc3C(=O)N3CC(F)(F)C3)cc(OC(C)C)n2)c(=O)n(C2CC2)c1] |
| 100 | 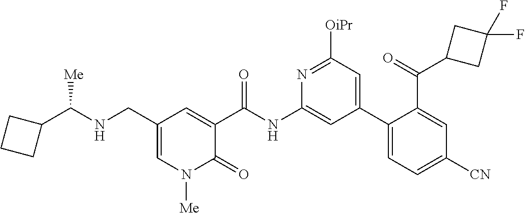 [SMILES: CC(C)Oc1cc(-c2ccc(C#N)cc2C(=O)C2CC(F)(F)C2)cc(NC(=O)c2cc(CN[C@@H](C)C3CCC3)cn(C)c2=O)n1] |
| 101 | 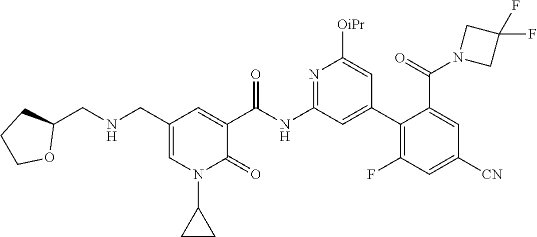 [SMILES: CC(C)Oc1cc(-c2c(F)cc(C#N)cc2C(=O)N2CC(F)(F)C2)cc(NC(=O)c2cc(CNC[C@@H]3CCCO3)cn(C3CC3)c2=O)n1] |
| 102 | 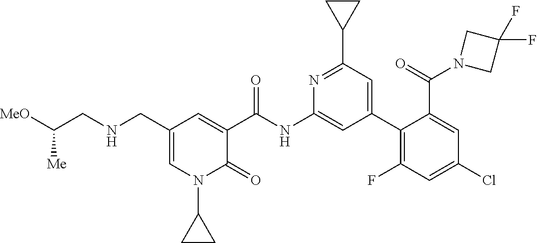 [SMILES: CO[C@@H](C)CNCc1cc(C(=O)Nc2cc(-c3c(F)cc(Cl)cc3C(=O)N3CC(F)(F)C3)cc(C3CC3)n2)c(=O)n(C2CC2)c1] |
| 103 | 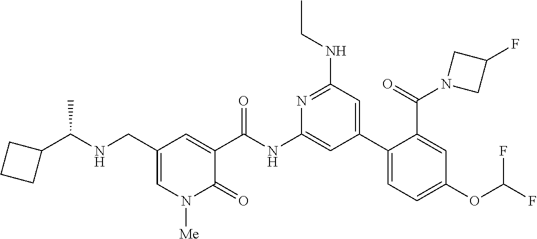 [SMILES: CCNc1cc(-c2ccc(OC(F)F)cc2C(=O)N2CC(F)C2)cc(NC(=O)c2cc(CN[C@@H](C)C3CCC3)cn(C)c2=O)n1] |
| 104 | 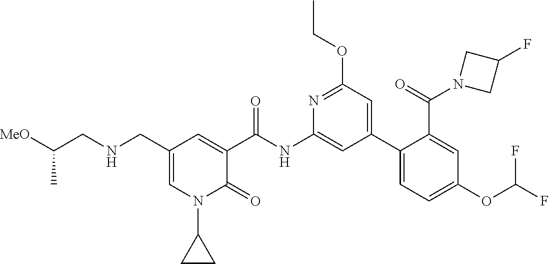 [SMILES: CCOc1cc(-c2ccc(OC(F)F)cc2C(=O)N2CC(F)C2)cc(NC(=O)c2cc(CNC[C@H](C)OC)cn(C3CC3)c2=O)n1] |

TABLE 4

| Example | Structure |
|---|---|
| 105 | 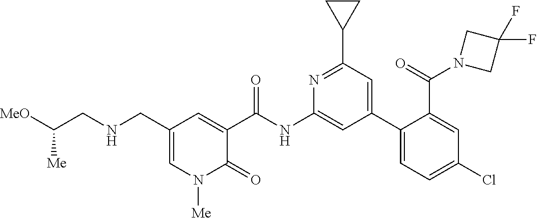 [SMILES: CO[C@@H](C)CNCc1cc(C(=O)Nc2cc(-c3ccc(Cl)cc3C(=O)N3CC(F)(F)C3)cc(C3CC3)n2)c(=O)n(C)c1] |
| 106 | 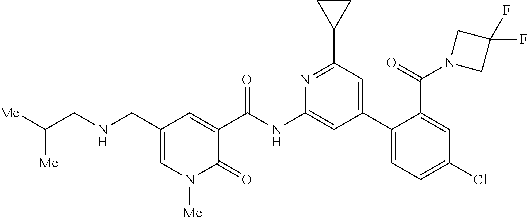 [SMILES: CC(C)CNCc1cc(C(=O)Nc2cc(-c3ccc(Cl)cc3C(=O)N3CC(F)(F)C3)cc(C3CC3)n2)c(=O)n(C)c1] |
| 107 | 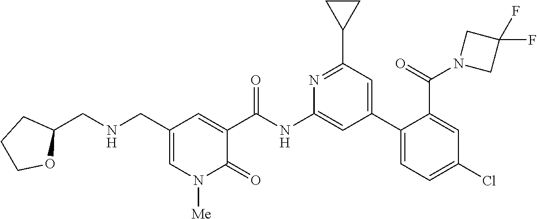 [SMILES: Cn1cc(CNC[C@@H]2CCCO2)cc(C(=O)Nc2cc(-c3ccc(Cl)cc3C(=O)N3CC(F)(F)C3)cc(C3CC3)n2)c1=O] |
| 108 | 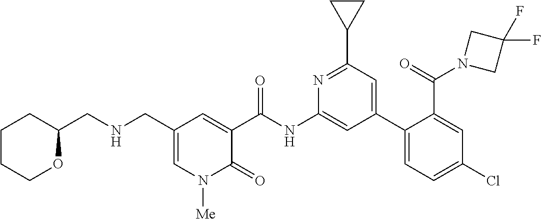 [SMILES: Cn1cc(CNC[C@@H]2CCCCO2)cc(C(=O)Nc2cc(-c3ccc(Cl)cc3C(=O)N3CC(F)(F)C3)cc(C3CC3)n2)c1=O] |
| 109 | 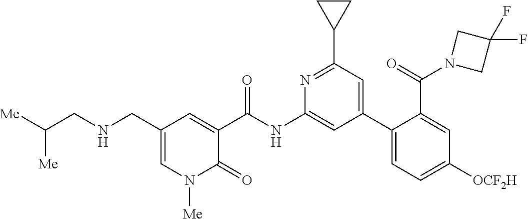 |
| 110 | 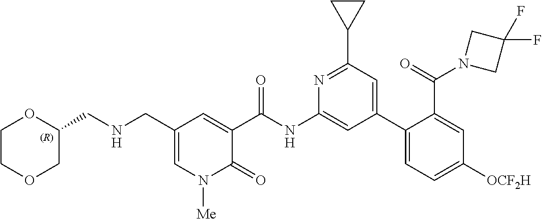 |
| 111 | 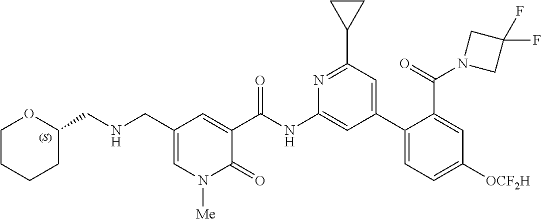 |
| 112 | 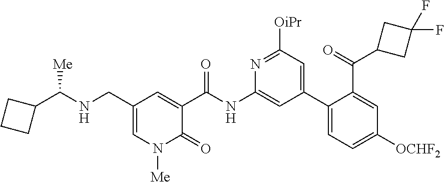 [SMILES: CC(C)Oc1cc(-c2ccc(OC(F)F)cc2C(=O)C2CC(F)(F)C2)cc(NC(=O)c2cc(CN[C@@H](C)C3CCC3)cn(C)c2=O)n1] |
| 113 | 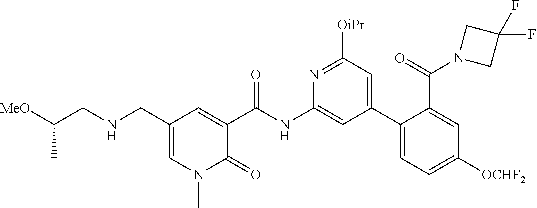 [SMILES: CO[C@@H](C)CNCc1cc(C(=O)Nc2cc(-c3ccc(OC(F)F)cc3C(=O)N3CC(F)(F)C3)cc(OC(C)C)n2)c(=O)n(C)c1] |
| 114 | 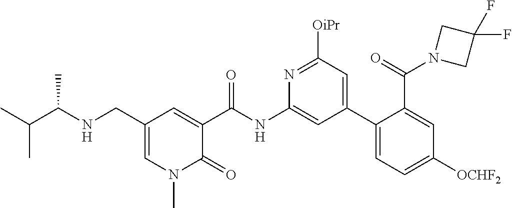 [SMILES: CC(C)Oc1cc(-c2ccc(OC(F)F)cc2C(=O)N2CC(F)(F)C2)cc(NC(=O)c2cc(CN[C@@H](C)C(C)C)cn(C)c2=O)n1] |
| 115 | 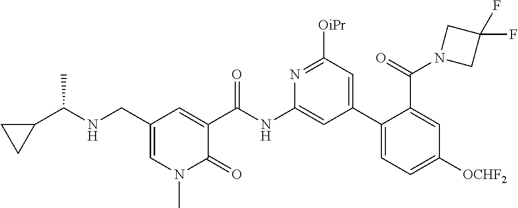 [SMILES: CC(C)Oc1cc(-c2ccc(OC(F)F)cc2C(=O)N2CC(F)(F)C2)cc(NC(=O)c2cc(CN[C@@H](C)C3CC3)cn(C)c2=O)n1] |
| 116 | 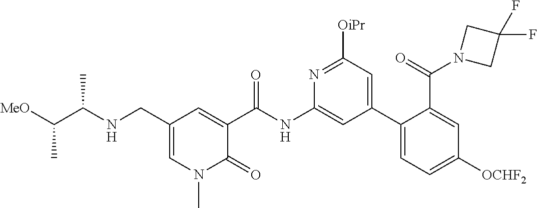 [SMILES: CO[C@@H](C)[C@H](C)NCc1cc(C(=O)Nc2cc(-c3ccc(OC(F)F)cc3C(=O)N3CC(F)(F)C3)cc(OC(C)C)n2)c(=O)n(C)c1] |
| 117 | 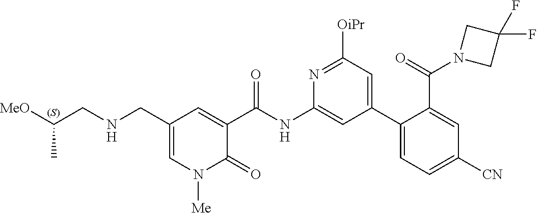 |
| 118 | 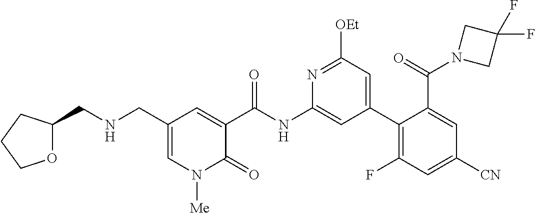 [SMILES: CCOc1cc(-c2c(F)cc(C#N)cc2C(=O)N2CC(F)(F)C2)cc(NC(=O)c2cc(CNC[C@@H]3CCCO3)cn(C)c2=O)n1] |
| 119 | 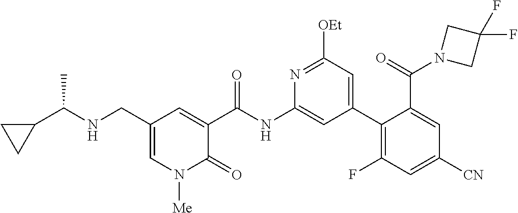 [SMILES: CCOc1cc(-c2c(F)cc(C#N)cc2C(=O)N2CC(F)(F)C2)cc(NC(=O)c2cc(CN[C@@H](C)C3CC3)cn(C)c2=O)n1] |
| 120 | 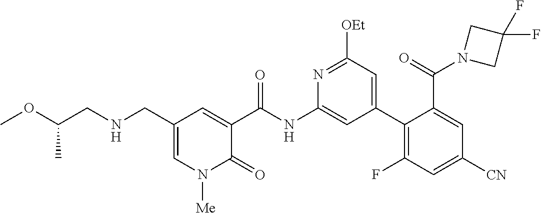 [SMILES: CCOc1cc(-c2c(F)cc(C#N)cc2C(=O)N2CC(F)(F)C2)cc(NC(=O)c2cc(CNC[C@H](C)OC)cn(C)c2=O)n1] |
| 121 | 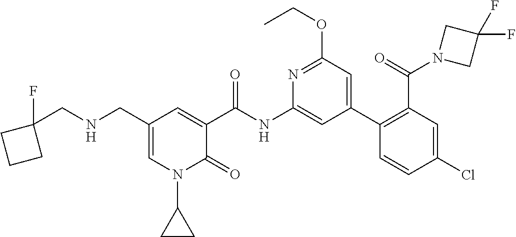 [SMILES: CCOc1cc(-c2ccc(Cl)cc2C(=O)N2CC(F)(F)C2)cc(NC(=O)c2cc(CNCC3(F)CCC3)cn(C3CC3)c2=O)n1] |
| 122 | 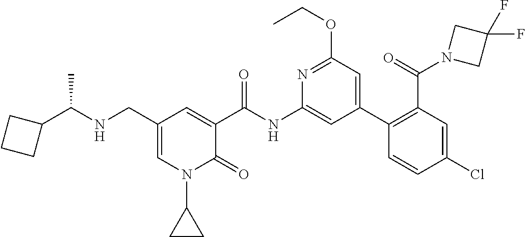 [SMILES: CCOc1cc(-c2ccc(Cl)cc2C(=O)N2CC(F)(F)C2)cc(NC(=O)c2cc(CN[C@@H](C)C3CCC3)cn(C3CC3)c2=O)n1] |
| 123 | 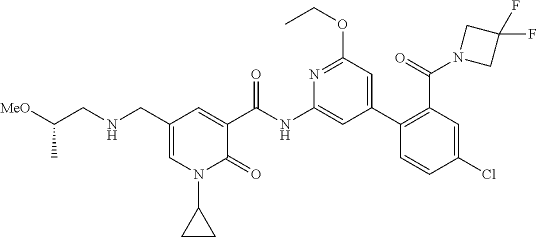 [SMILES: CCOc1cc(-c2ccc(Cl)cc2C(=O)N2CC(F)(F)C2)cc(NC(=O)c2cc(CNC[C@H](C)OC)cn(C3CC3)c2=O)n1] |
| 124 | 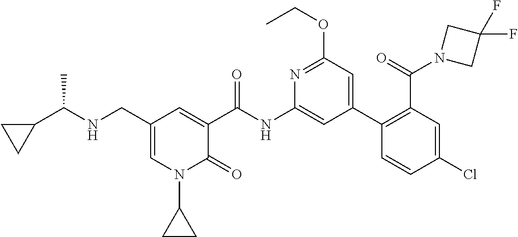 [SMILES: CCOc1cc(-c2ccc(Cl)cc2C(=O)N2CC(F)(F)C2)cc(NC(=O)c2cc(CN[C@@H](C)C3CC3)cn(C3CC3)c2=O)n1] |
| 125 | 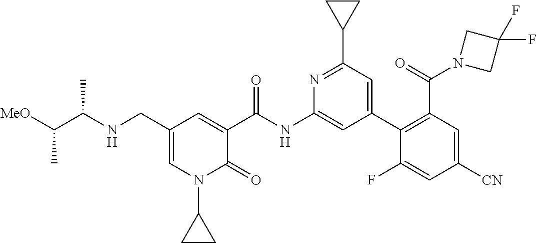 [SMILES: CO[C@@H](C)[C@H](C)NCc1cc(C(=O)Nc2cc(-c3c(F)cc(C#N)cc3C(=O)N3CC(F)(F)C3)cc(C3CC3)n2)c(=O)n(C2CC2)c1] |
| 126 | 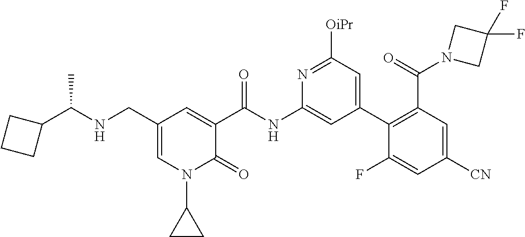 [SMILES: CC(C)Oc1cc(-c2c(F)cc(C#N)cc2C(=O)N2CC(F)(F)C2)cc(NC(=O)c2cc(CN[C@@H](C)C3CCC3)cn(C3CC3)c2=O)n1] |
| 127 | 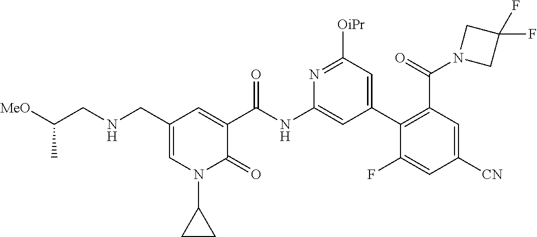 [SMILES: CO[C@@H](C)CNCc1cc(C(=O)Nc2cc(-c3c(F)cc(C#N)cc3C(=O)N3CC(F)(F)C3)cc(OC(C)C)n2)c(=O)n(C2CC2)c1] |
| 128 | 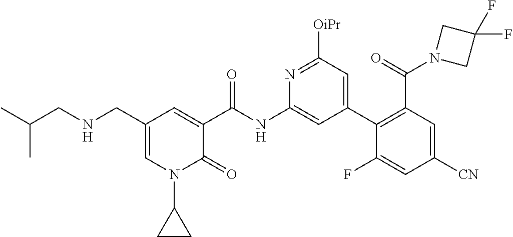 [SMILES: CC(C)CNCc1cc(C(=O)Nc2cc(-c3c(F)cc(C#N)cc3C(=O)N3CC(F)(F)C3)cc(OC(C)C)n2)c(=O)n(C2CC2)c1] |
| 129 | 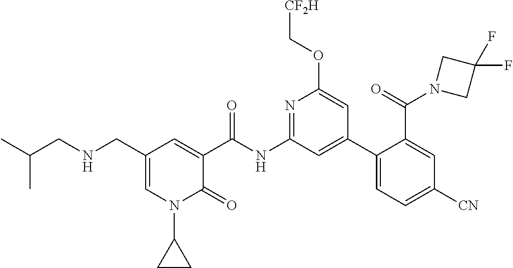 [SMILES: CC(C)CNCc1cc(C(=O)Nc2cc(-c3ccc(C#N)cc3C(=O)N3CC(F)(F)C3)cc(OCC(F)F)n2)c(=O)n(C2CC2)c1] |
| 130 | 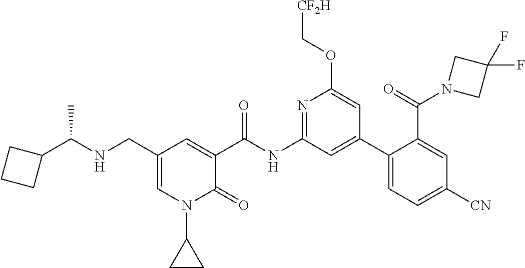 [SMILES: C[C@H](NCc1cc(C(=O)Nc2cc(-c3ccc(C#N)cc3C(=O)N3CC(F)(F)C3)cc(OCC(F)F)n2)c(=O)n(C2CC2)c1)C1CCC1] |
| 131 | 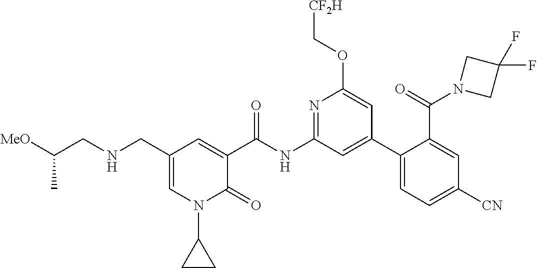 [SMILES: CO[C@@H](C)CNCc1cc(C(=O)Nc2cc(-c3ccc(C#N)cc3C(=O)N3CC(F)(F)C3)cc(OCC(F)F)n2)c(=O)n(C2CC2)c1] |
| 132 | 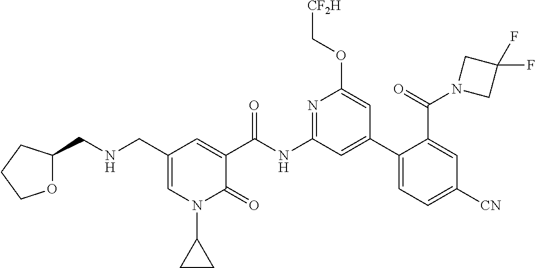 [SMILES: N#Cc1ccc(-c2cc(NC(=O)c3cc(CNC[C@@H]4CCCO4)cn(C4CC4)c3=O)nc(OCC(F)F)c2)c(C(=O)N2CC(F)(F)C2)c1] |
| 133 | 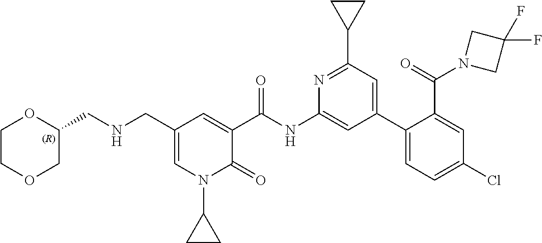 |
| 134 | 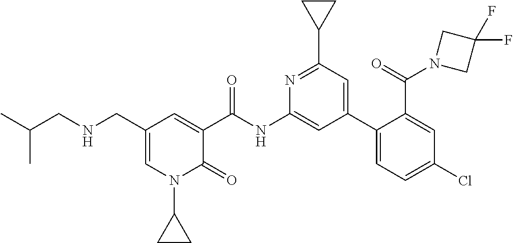 [SMILES: CC(C)CNCc1cc(C(=O)Nc2cc(-c3ccc(Cl)cc3C(=O)N3CC(F)(F)C3)cc(C3CC3)n2)c(=O)n(C2CC2)c1] |
| 135 | 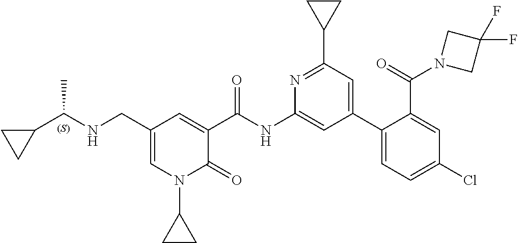 |
| 136 | 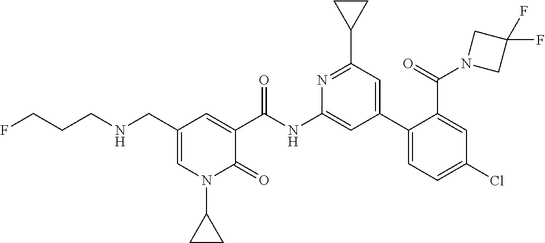 [SMILES: O=C(Nc1cc(-c2ccc(Cl)cc2C(=O)N2CC(F)(F)C2)cc(C2CC2)n1)c1cc(CNCCCF)cn(C2CC2)c1=O] |
| 137 | 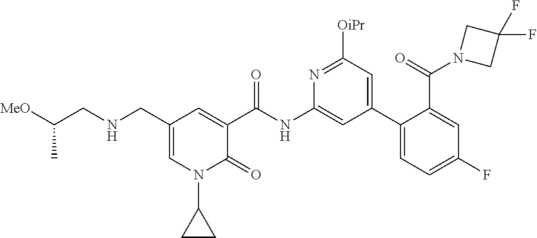 [SMILES: CO[C@@H](C)CNCc1cc(C(=O)Nc2cc(-c3ccc(F)cc3C(=O)N3CC(F)(F)C3)cc(OC(C)C)n2)c(=O)n(C2CC2)c1] |
| 138 | 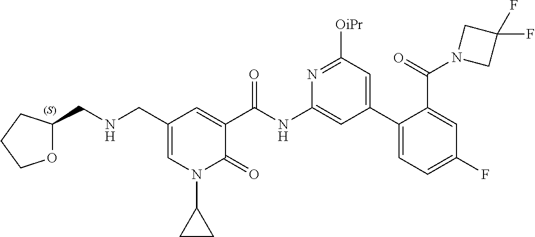 |
| 139 | 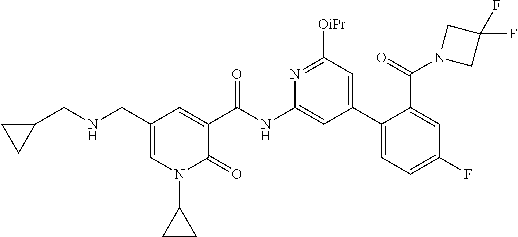 [SMILES: CC(C)Oc1cc(-c2ccc(F)cc2C(=O)N2CC(F)(F)C2)cc(NC(=O)c2cc(CNCC3CC3)cn(C3CC3)c2=O)n1] |
| 140 | 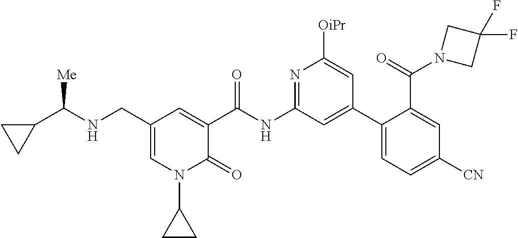 [SMILES: CC(C)Oc1cc(-c2ccc(C#N)cc2C(=O)N2CC(F)(F)C2)cc(NC(=O)c2cc(CN[C@H](C)C3CC3)cn(C3CC3)c2=O)n1] |
| 141 | 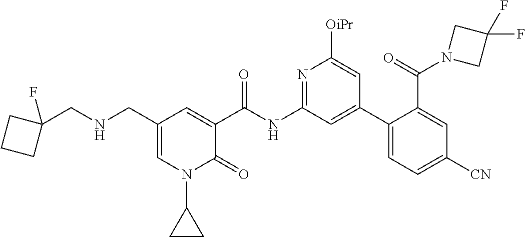 [SMILES: CC(C)Oc1cc(-c2ccc(C#N)cc2C(=O)N2CC(F)(F)C2)cc(NC(=O)c2cc(CNCC3(F)CCC3)cn(C3CC3)c2=O)n1] |
| 142 | 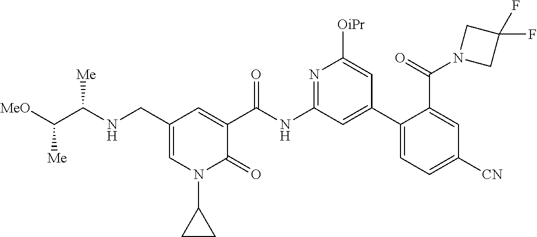 [SMILES: CO[C@@H](C)[C@H](C)NCc1cc(C(=O)Nc2cc(-c3ccc(C#N)cc3C(=O)N3CC(F)(F)C3)cc(OC(C)C)n2)c(=O)n(C2CC2)c1] |
| 143 | 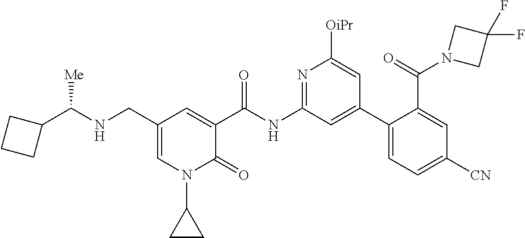 [SMILES: CC(C)Oc1cc(-c2ccc(C#N)cc2C(=O)N2CC(F)(F)C2)cc(NC(=O)c2cc(CN[C@@H](C)C3CCC3)cn(C3CC3)c2=O)n1] |
| 144 | 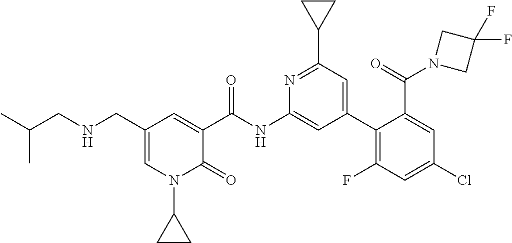 [SMILES: CC(C)CNCc1cc(C(=O)Nc2cc(-c3c(F)cc(Cl)cc3C(=O)N3CC(F)(F)C3)cc(C3CC3)n2)c(=O)n(C2CC2)c1] |
| 145 | 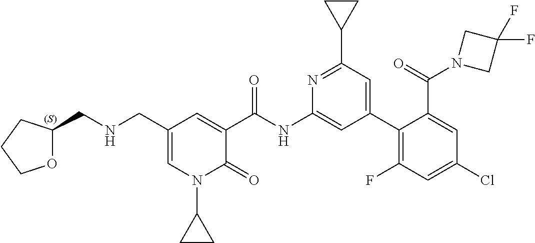 |
| 146 | 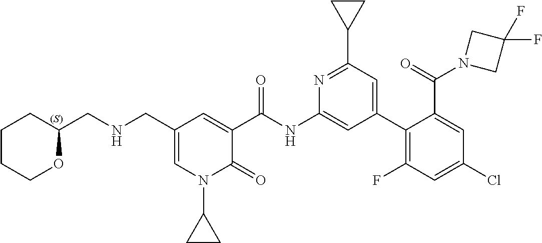 |
| 147 | 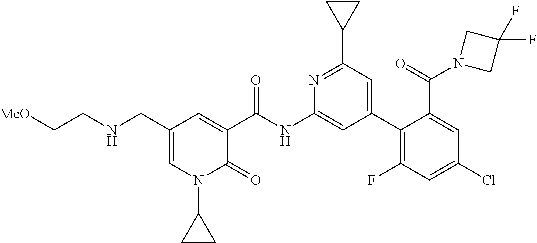 [SMILES: COCCNCc1cc(C(=O)Nc2cc(-c3c(F)cc(Cl)cc3C(=O)N3CC(F)(F)C3)cc(C3CC3)n2)c(=O)n(C2CC2)c1] |
| 148 | 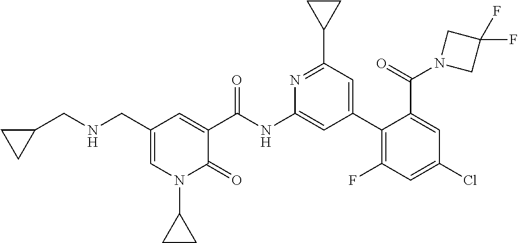 [SMILES: O=C(Nc1cc(-c2c(F)cc(Cl)cc2C(=O)N2CC(F)(F)C2)cc(C2CC2)n1)c1cc(CNCC2CC2)cn(C2CC2)c1=O] |
| 149 | 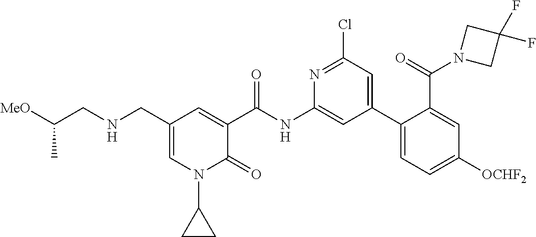 [SMILES: CO[C@@H](C)CNCc1cc(C(=O)Nc2cc(-c3ccc(OC(F)F)cc3C(=O)N3CC(F)(F)C3)cc(Cl)n2)c(=O)n(C2CC2)c1] |
| 150 | 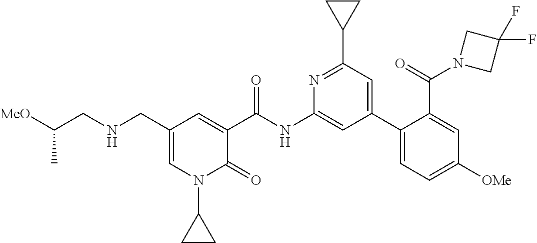 [SMILES: COc1ccc(-c2cc(NC(=O)c3cc(CNC[C@H](C)OC)cn(C4CC4)c3=O)nc(C3CC3)c2)c(C(=O)N2CC(F)(F)C2)c1] |
| 151 | 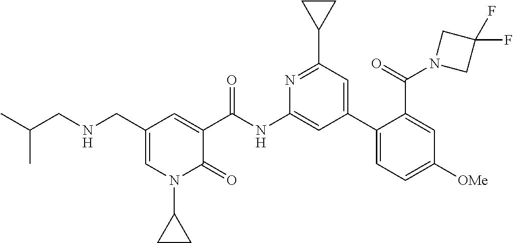 [SMILES: COc1ccc(-c2cc(NC(=O)c3cc(CNCC(C)C)cn(C4CC4)c3=O)nc(C3CC3)c2)c(C(=O)N2CC(F)(F)C2)c1] |
| 152 | 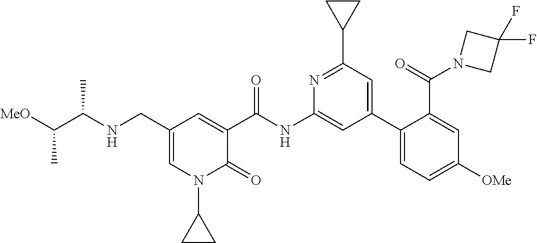 [SMILES: COc1ccc(-c2cc(NC(=O)c3cc(CN[C@@H](C)[C@H](C)OC)cn(C4CC4)c3=O)nc(C3CC3)c2)c(C(=O)N2CC(F)(F)C2)c1] |
| 153 | 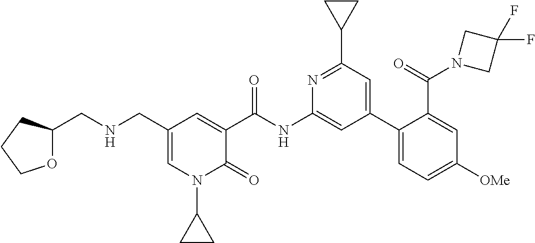 [SMILES: COc1ccc(-c2cc(NC(=O)c3cc(CNC[C@@H]4CCCO4)cn(C4CC4)c3=O)nc(C3CC3)c2)c(C(=O)N2CC(F)(F)C2)c1] |
| 154 | 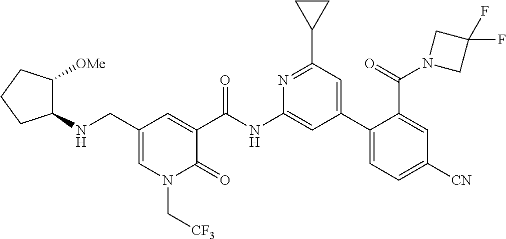 [SMILES: CO[C@H]1CCC[C@@H]1NCc1cc(C(=O)Nc2cc(-c3ccc(C#N)cc3C(=O)N3CC(F)(F)C3)cc(C3CC3)n2)c(=O)n(CC(F)(F)F)c1] |
| 155 | 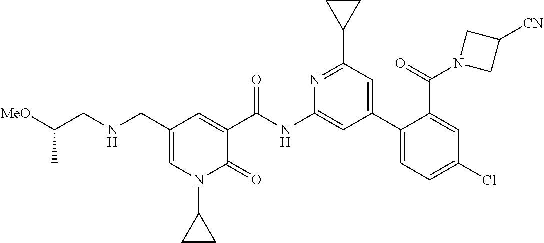 [SMILES: CO[C@@H](C)CNCc1cc(C(=O)Nc2cc(-c3ccc(Cl)cc3C(=O)N3CC(C#N)C3)cc(C3CC3)n2)c(=O)n(C2CC2)c1] |
| 156 | 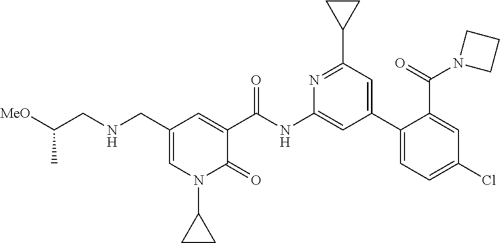 [SMILES: CO[C@@H](C)CNCc1cc(C(=O)Nc2cc(-c3ccc(Cl)cc3C(=O)N3CCC3)cc(C3CC3)n2)c(=O)n(C2CC2)c1] |
| 157 | 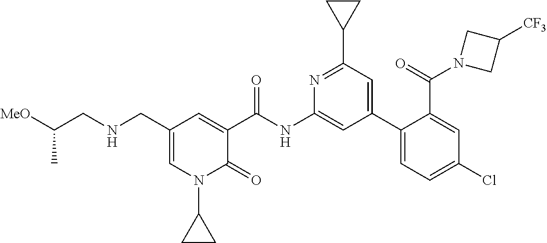 [SMILES: CO[C@@H](C)CNCc1cc(C(=O)Nc2cc(-c3ccc(Cl)cc3C(=O)N3CC(C(F)(F)F)C3)cc(C3CC3)n2)c(=O)n(C2CC2)c1] |
| 158 | 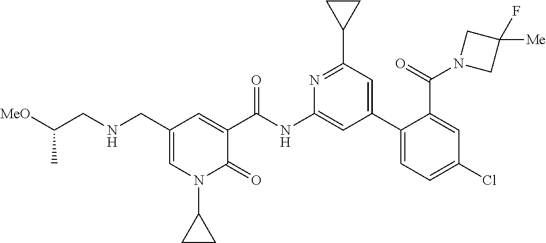 [SMILES: CO[C@@H](C)CNCc1cc(C(=O)Nc2cc(-c3ccc(Cl)cc3C(=O)N3CC(C)(F)C3)cc(C3CC3)n2)c(=O)n(C2CC2)c1] |
| 159 | 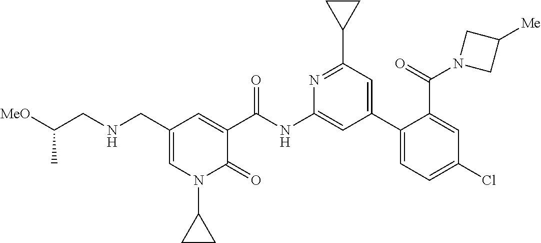 [SMILES: CO[C@@H](C)CNCc1cc(C(=O)Nc2cc(-c3ccc(Cl)cc3C(=O)N3CC(C)C3)cc(C3CC3)n2)c(=O)n(C2CC2)c1] |
| 160 | 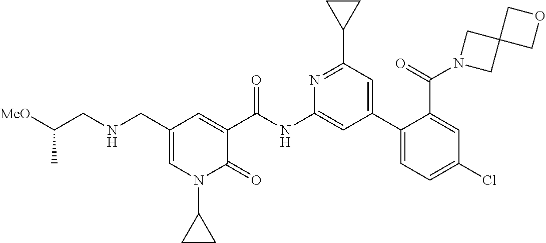 [SMILES: CO[C@@H](C)CNCc1cc(C(=O)Nc2cc(-c3ccc(Cl)cc3C(=O)N3CC4(COC4)C3)cc(C3CC3)n2)c(=O)n(C2CC2)c1] |
| 161 | 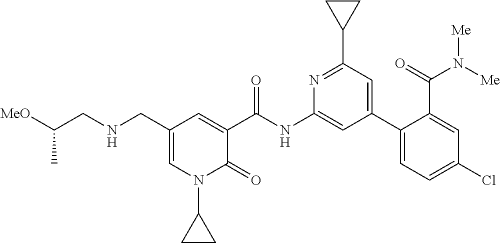 [SMILES: CO[C@@H](C)CNCc1cc(C(=O)Nc2cc(-c3ccc(Cl)cc3C(=O)N(C)C)cc(C3CC3)n2)c(=O)n(C2CC2)c1] |

TABLE 5

| Example | Structure |
|---|---|
| 162 | 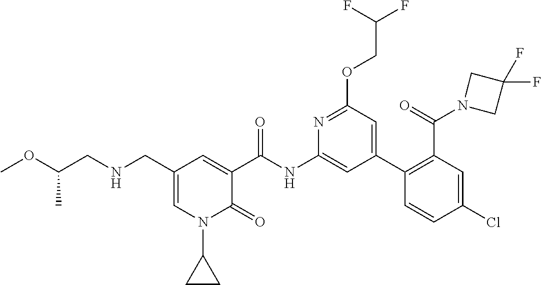 [SMILES: CO[C@@H](C)CNCc1cc(C(=O)Nc2cc(-c3ccc(Cl)cc3C(=O)N3CC(F)(F)C3)cc(OCC(F)F)n2)c(=O)n(C2CC2)c1] |
| 163 | 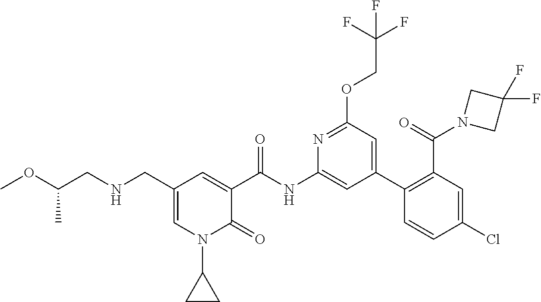 [SMILES: CO[C@@H](C)CNCc1cc(C(=O)Nc2cc(-c3ccc(Cl)cc3C(=O)N3CC(F)(F)C3)cc(OCC(F)(F)F)n2)c(=O)n(C2CC2)c1] |
| 164 | 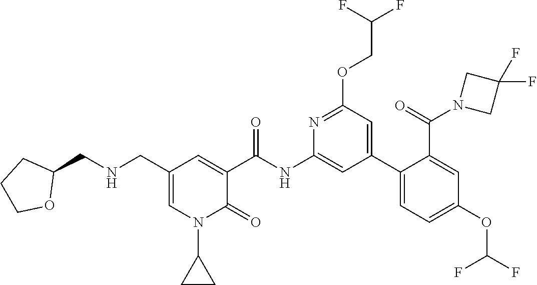 [SMILES: O=C(Nc1cc(-c2ccc(OC(F)F)cc2C(=O)N2CC(F)(F)C2)cc(OCC(F)F)n1)c1cc(CNC[C@@H]2CCCO2)cn(C2CC2)c1=O] |
| 165 | 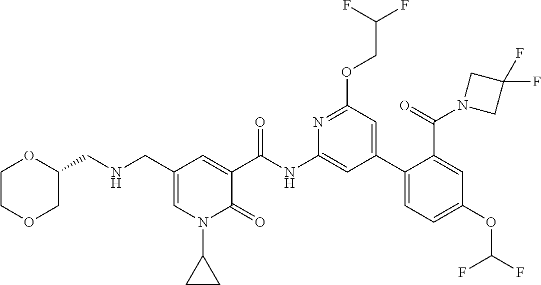 [SMILES: O=C(Nc1cc(-c2ccc(OC(F)F)cc2C(=O)N2CC(F)(F)C2)cc(OCC(F)F)n1)c1cc(CNC[C@@H]2COCCO2)cn(C2CC2)c1=O] |
| 166 | 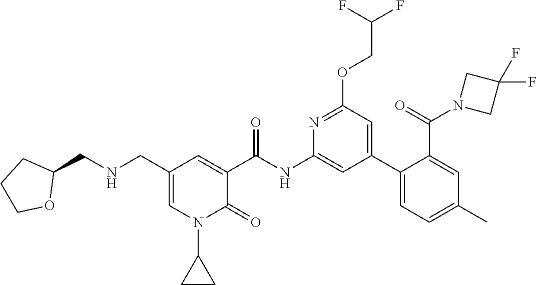 [SMILES: Cc1ccc(-c2cc(NC(=O)c3cc(CNC[C@@H]4CCCO4)cn(C4CC4)c3=O)nc(OCC(F)F)c2)c(C(=O)N2CC(F)(F)C2)c1] |
| 167 | 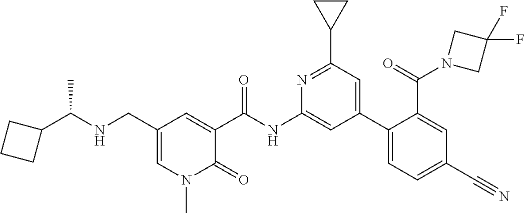 [SMILES: C[C@H](NCc1cc(C(=O)Nc2cc(-c3ccc(C#N)cc3C(=O)N3CC(F)(F)C3)cc(C3CC3)n2)c(=O)n(C)c1)C1CCC1] |
| 168 | 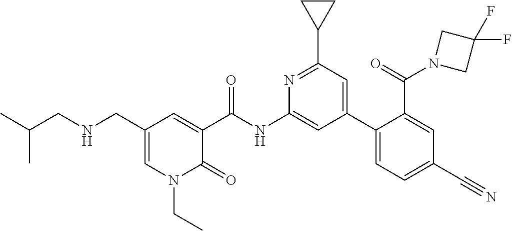 [SMILES: CCn1cc(CNCC(C)C)cc(C(=O)Nc2cc(-c3ccc(C#N)cc3C(=O)N3CC(F)(F)C3)cc(C3CC3)n2)c1=O] |
| 169 | 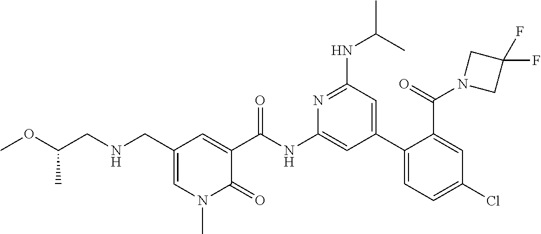 [SMILES: CO[C@@H](C)CNCc1cc(C(=O)Nc2cc(-c3ccc(Cl)cc3C(=O)N3CC(F)(F)C3)cc(NC(C)C)n2)c(=O)n(C)c1] |
| 170 | 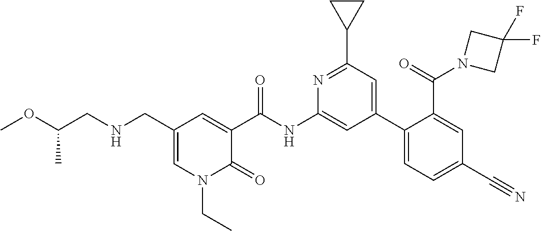 [SMILES: CCn1cc(CNC[C@H](C)OC)cc(C(=O)Nc2cc(-c3ccc(C#N)cc3C(=O)N3CC(F)(F)C3)cc(C3CC3)n2)c1=O] |
| 171 | 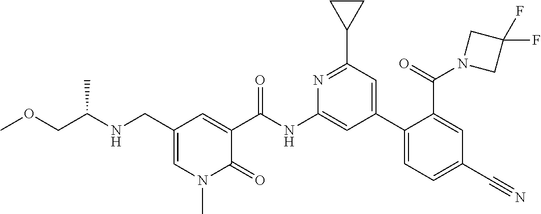 [SMILES: COC[C@H](C)NCc1cc(C(=O)Nc2cc(-c3ccc(C#N)cc3C(=O)N3CC(F)(F)C3)cc(C3CC3)n2)c(=O)n(C)c1] |
| 172 | 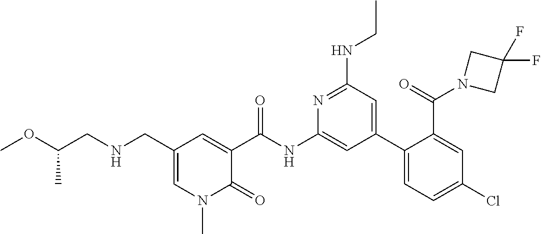 [SMILES: CCNc1cc(-c2ccc(Cl)cc2C(=O)N2CC(F)(F)C2)cc(NC(=O)c2cc(CNC[C@H](C)OC)cn(C)c2=O)n1] |
| 173 | 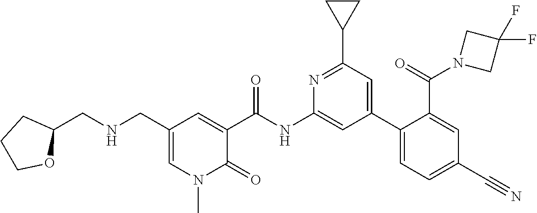 [SMILES: Cn1cc(CNC[C@@H]2CCCO2)cc(C(=O)Nc2cc(-c3ccc(C#N)cc3C(=O)N3CC(F)(F)C3)cc(C3CC3)n2)c1=O] |
| 174 | 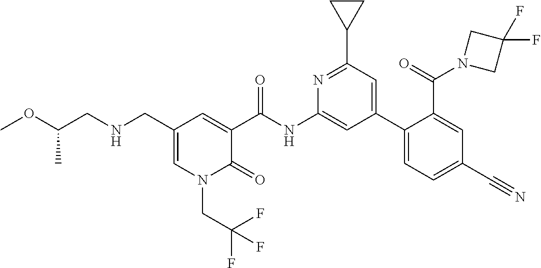 [SMILES: CO[C@@H](C)CNCc1cc(C(=O)Nc2cc(-c3ccc(C#N)cc3C(=O)N3CC(F)(F)C3)cc(C3CC3)n2)c(=O)n(CC(F)(F)F)c1] |
| 175 | 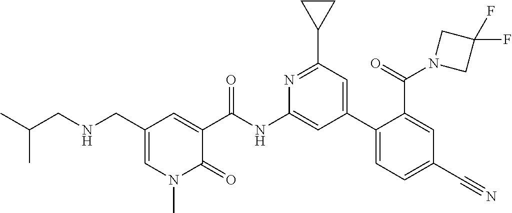 [SMILES: CC(C)CNCc1cc(C(=O)Nc2cc(-c3ccc(C#N)cc3C(=O)N3CC(F)(F)C3)cc(C3CC3)n2)c(=O)n(C)c1] |
| 176 | 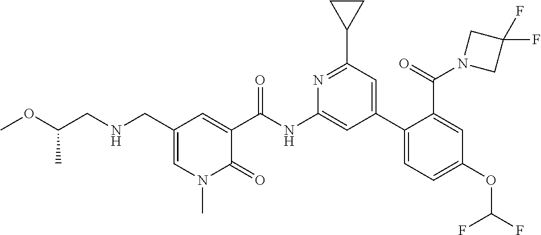 [SMILES: CO[C@@H](C)CNCc1cc(C(=O)Nc2cc(-c3ccc(OC(F)F)cc3C(=O)N3CC(F)(F)C3)cc(C3CC3)n2)c(=O)n(C)c1] |
| 177 | 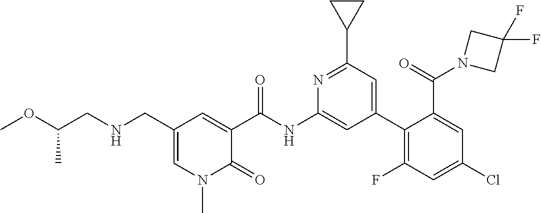 [SMILES: CO[C@@H](C)CNCc1cc(C(=O)Nc2cc(-c3c(F)cc(Cl)cc3C(=O)N3CC(F)(F)C3)cc(C3CC3)n2)c(=O)n(C)c1] |
| 178 | 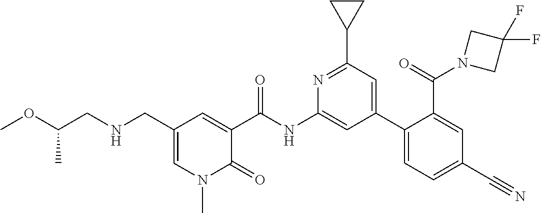 [SMILES: CO[C@@H](C)CNCc1cc(C(=O)Nc2cc(-c3ccc(C#N)cc3C(=O)N3CC(F)(F)C3)cc(C3CC3)n2)c(=O)n(C)c1] |
| 179 | 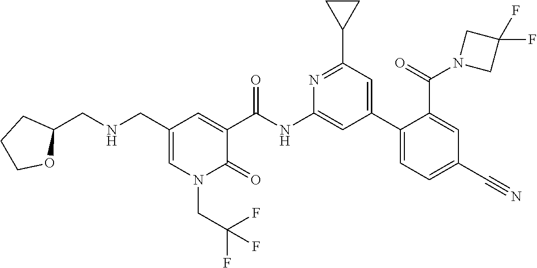 [SMILES: N#Cc1ccc(-c2cc(NC(=O)c3cc(CNC[C@@H]4CCCO4)cn(CC(F)(F)F)c3=O)nc(C3CC3)c2)c(C(=O)N2CC(F)(F)C2)c1] |
| 180 | 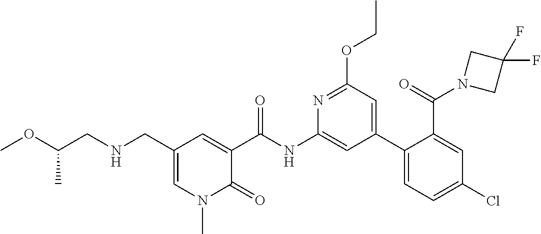 [SMILES: CCOc1cc(-c2ccc(Cl)cc2C(=O)N2CC(F)(F)C2)cc(NC(=O)c2cc(CNC[C@H](C)OC)cn(C)c2=O)n1] |
| 181 | 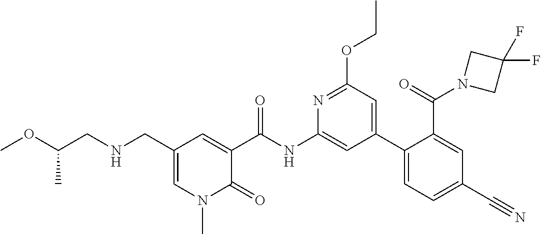 [SMILES: CCOc1cc(-c2ccc(C#N)cc2C(=O)N2CC(F)(F)C2)cc(NC(=O)c2cc(CNC[C@H](C)OC)cn(C)c2=O)n1] |
| 182 | 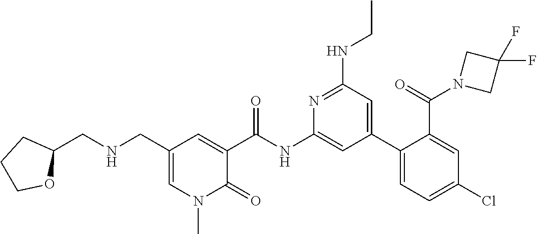 [SMILES: CCNc1cc(-c2ccc(Cl)cc2C(=O)N2CC(F)(F)C2)cc(NC(=O)c2cc(CNC[C@@H]3CCCO3)cn(C)c2=O)n1] |
| 183 | 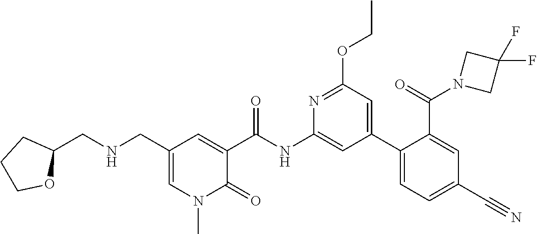 [SMILES: CCOc1cc(-c2ccc(C#N)cc2C(=O)N2CC(F)(F)C2)cc(NC(=O)c2cc(CNC[C@@H]3CCCO3)cn(C)c2=O)n1] |
| 184 | 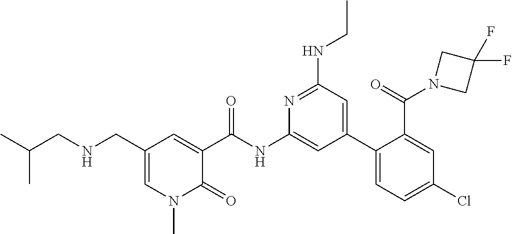 [SMILES: CCNc1cc(-c2ccc(Cl)cc2C(=O)N2CC(F)(F)C2)cc(NC(=O)c2cc(CNCC(C)C)cn(C)c2=O)n1] |
| 185 | 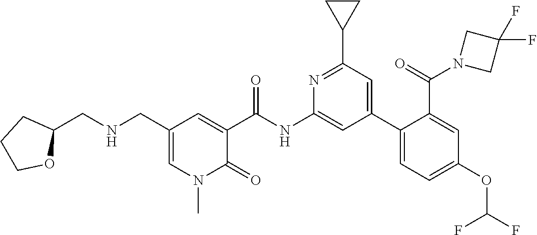 [SMILES: Cn1cc(CNC[C@@H]2CCCO2)cc(C(=O)Nc2cc(-c3ccc(OC(F)F)cc3C(=O)N3CC(F)(F)C3)cc(C3CC3)n2)c1=O] |
| 186 | 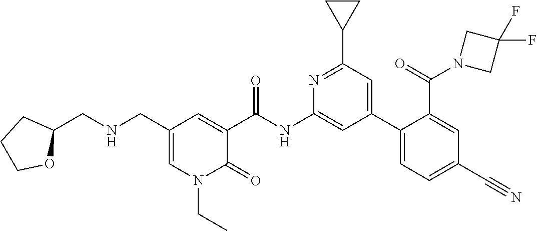 [SMILES: CCn1cc(CNC[C@@H]2CCCO2)cc(C(=O)Nc2cc(-c3ccc(C#N)cc3C(=O)N3CC(F)(F)C3)cc(C3CC3)n2)c1=O] |
| 187 | 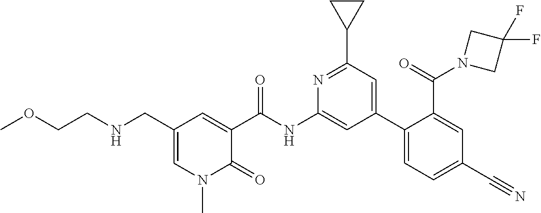 [SMILES: COCCNCc1cc(C(=O)Nc2cc(-c3ccc(C#N)cc3C(=O)N3CC(F)(F)C3)cc(C3CC3)n2)c(=O)n(C)c1] |
| 188 | 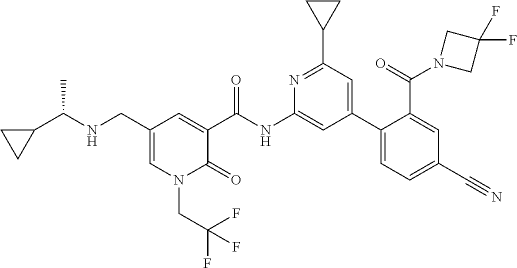 [SMILES: C[C@H](NCc1cc(C(=O)Nc2cc(-c3ccc(C#N)cc3C(=O)N3CC(F)(F)C3)cc(C3CC3)n2)c(=O)n(CC(F)(F)F)c1)C1CC1] |
| 189 | 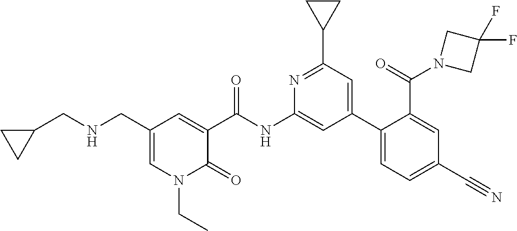 [SMILES: CCn1cc(CNCC2CC2)cc(C(=O)Nc2cc(-c3ccc(C#N)cc3C(=O)N3CC(F)(F)C3)cc(C3CC3)n2)c1=O] |
| 190 | 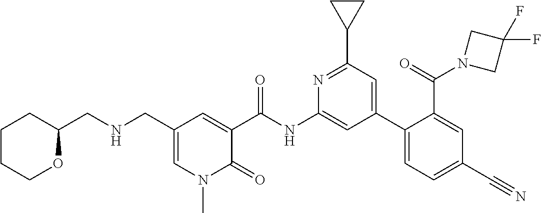 [SMILES: Cn1cc(CNC[C@@H]2CCCCO2)cc(C(=O)Nc2cc(-c3ccc(C#N)cc3C(=O)N3CC(F)(F)C3)cc(C3CC3)n2)c1=O] |
| 191 | 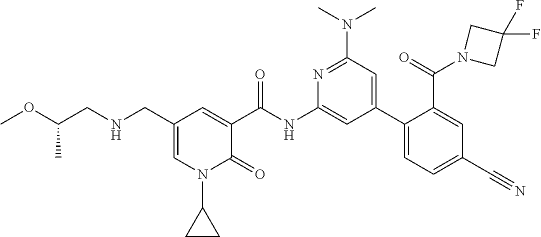 [SMILES: CO[C@@H](C)CNCc1cc(C(=O)Nc2cc(-c3ccc(C#N)cc3C(=O)N3CC(F)(F)C3)cc(N(C)C)n2)c(=O)n(C2CC2)c1] |
| 192 | 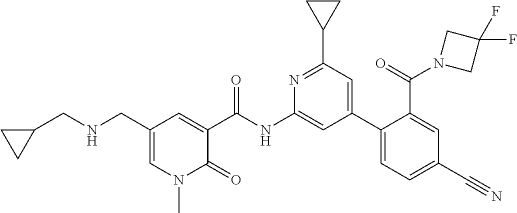 [SMILES: Cn1cc(CNCC2CC2)cc(C(=O)Nc2cc(-c3ccc(C#N)cc3C(=O)N3CC(F)(F)C3)cc(C3CC3)n2)c1=O] |
| 193 | 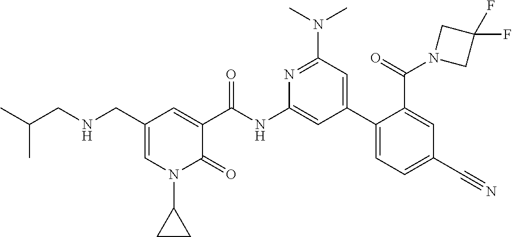 [SMILES: CC(C)CNCc1cc(C(=O)Nc2cc(-c3ccc(C#N)cc3C(=O)N3CC(F)(F)C3)cc(N(C)C)n2)c(=O)n(C2CC2)c1] |
| 194 | 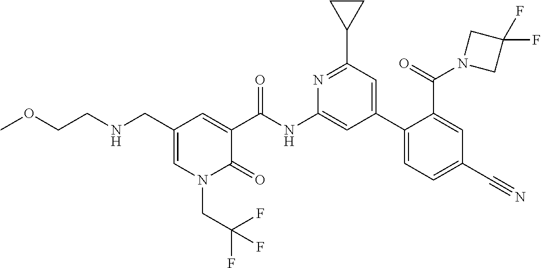 [SMILES: COCCNCc1cc(C(=O)Nc2cc(-c3ccc(C#N)cc3C(=O)N3CC(F)(F)C3)cc(C3CC3)n2)c(=O)n(CC(F)(F)F)c1] |
| 195 | 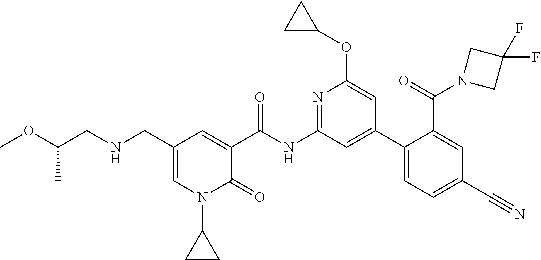 [SMILES: CO[C@@H](C)CNCc1cc(C(=O)Nc2cc(-c3ccc(C#N)cc3C(=O)N3CC(F)(F)C3)cc(OC3CC3)n2)c(=O)n(C2CC2)c1] |
| 196 | 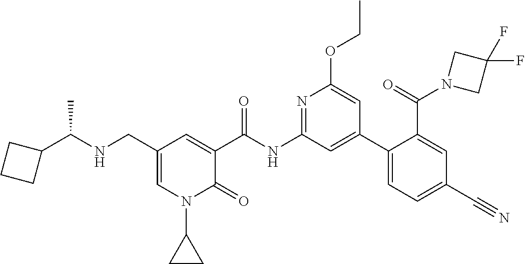 [SMILES: CCOc1cc(-c2ccc(C#N)cc2C(=O)N2CC(F)(F)C2)cc(NC(=O)c2cc(CN[C@@H](C)C3CCC3)cn(C3CC3)c2=O)n1] |
| 197 | 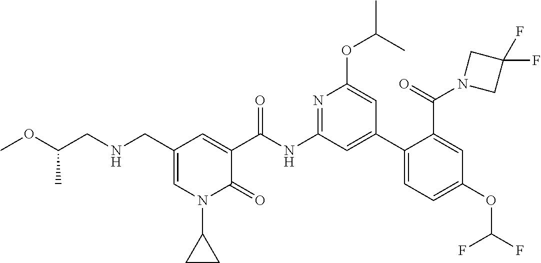 [SMILES: CO[C@@H](C)CNCc1cc(C(=O)Nc2cc(-c3ccc(OC(F)F)cc3C(=O)N3CC(F)(F)C3)cc(OC(C)C)n2)c(=O)n(C2CC2)c1] |
| 198 | 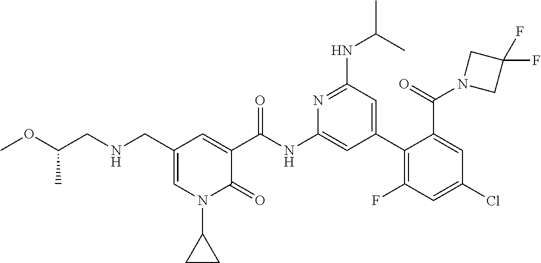 [SMILES: CO[C@@H](C)CNCc1cc(C(=O)Nc2cc(-c3c(F)cc(Cl)cc3C(=O)N3CC(F)(F)C3)cc(NC(C)C)n2)c(=O)n(C2CC2)c1] |
| 199 | 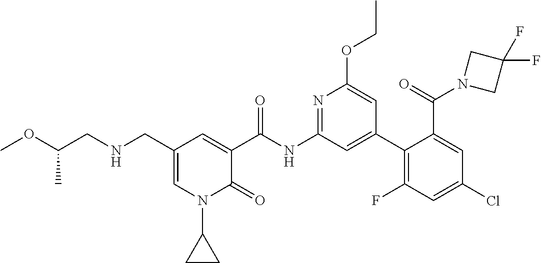 [SMILES: CCOc1cc(-c2c(F)cc(Cl)cc2C(=O)N2CC(F)(F)C2)cc(NC(=O)c2cc(CNC[C@H](C)OC)cn(C3CC3)c2=O)n1] |
| 200 | 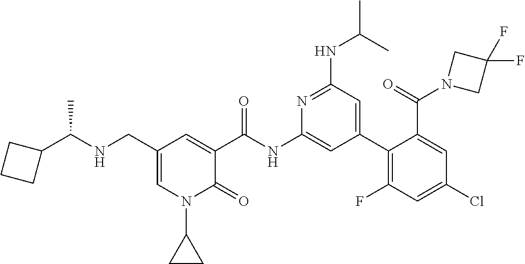 [SMILES: CC(C)Nc1cc(-c2c(F)cc(Cl)cc2C(=O)N2CC(F)(F)C2)cc(NC(=O)c2cc(CN[C@@H](C)C3CCC3)cn(C3CC3)c2=O)n1] |
| 201 | 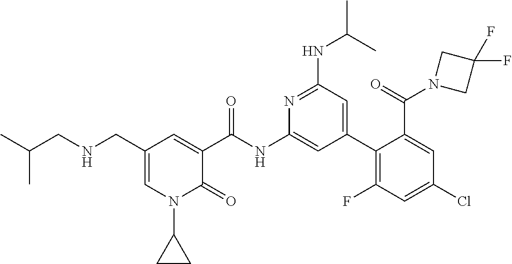 [SMILES: CC(C)CNCc1cc(C(=O)Nc2cc(-c3c(F)cc(Cl)cc3C(=O)N3CC(F)(F)C3)cc(NC(C)C)n2)c(=O)n(C2CC2)c1] |
| 202 | 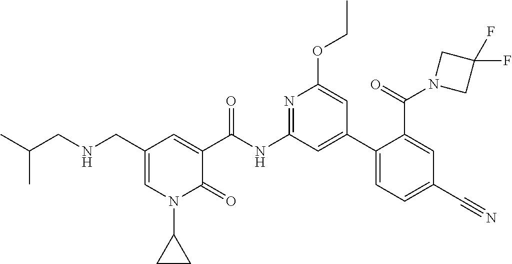 [SMILES: CCOc1cc(-c2ccc(C#N)cc2C(=O)N2CC(F)(F)C2)cc(NC(=O)c2cc(CNCC(C)C)cn(C3CC3)c2=O)n1] |
| 203 | 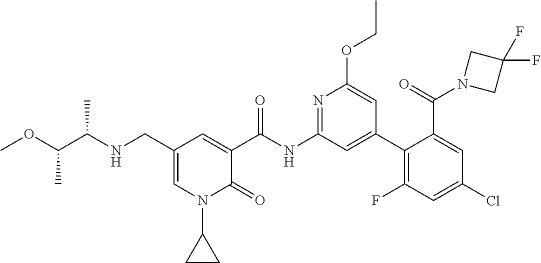 [SMILES: CCOc1cc(-c2c(F)cc(Cl)cc2C(=O)N2CC(F)(F)C2)cc(NC(=O)c2cc(CN[C@@H](C)[C@H](C)OC)cn(C3CC3)c2=O)n1] |
| 204 | 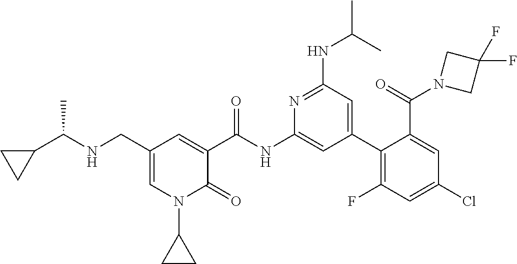 [SMILES: CC(C)Nc1cc(-c2c(F)cc(Cl)cc2C(=O)N2CC(F)(F)C2)cc(NC(=O)c2cc(CN[C@@H](C)C3CC3)cn(C3CC3)c2=O)n1] |
| 205 | 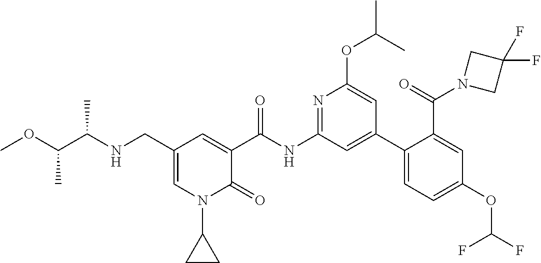 [SMILES: CO[C@@H](C)[C@H](C)NCc1cc(C(=O)Nc2cc(-c3ccc(OC(F)F)cc3C(=O)N3CC(F)(F)C3)cc(OC(C)C)n2)c(=O)n(C2CC2)c1] |
| 206 | 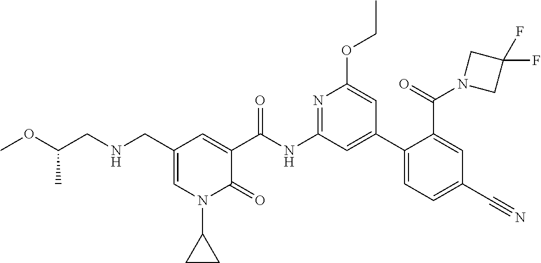 [SMILES: CCOc1cc(-c2ccc(C#N)cc2C(=O)N2CC(F)(F)C2)cc(NC(=O)c2cc(CNC[C@H](C)OC)cn(C3CC3)c2=O)n1] |
| 207 | 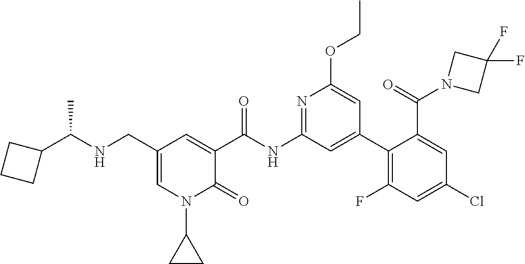 [SMILES: CCOc1cc(-c2c(F)cc(Cl)cc2C(=O)N2CC(F)(F)C2)cc(NC(=O)c2cc(CN[C@@H](C)C3CCC3)cn(C3CC3)c2=O)n1] |
| 208 | 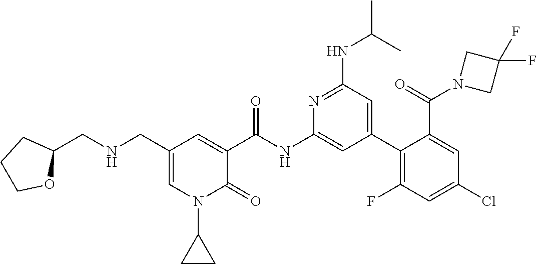 [SMILES: CC(C)Nc1cc(-c2c(F)cc(Cl)cc2C(=O)N2CC(F)(F)C2)cc(NC(=O)c2cc(CNC[C@@H]3CCCO3)cn(C3CC3)c2=O)n1] |
| 209 | 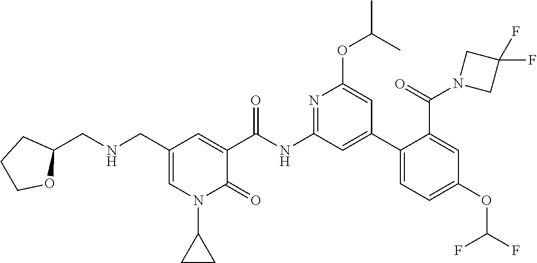 [SMILES: CC(C)Oc1cc(-c2ccc(OC(F)F)cc2C(=O)N2CC(F)(F)C2)cc(NC(=O)c2cc(CNC[C@@H]3CCCO3)cn(C3CC3)c2=O)n1] |
| 210 | 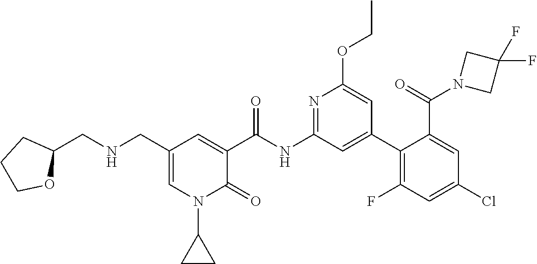 [SMILES: CCOc1cc(-c2c(F)cc(Cl)cc2C(=O)N2CC(F)(F)C2)cc(NC(=O)c2cc(CNC[C@@H]3CCCO3)cn(C3CC3)c2=O)n1] |
| 211 | 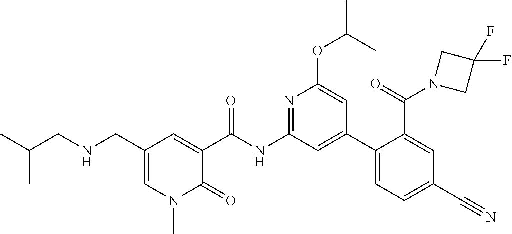 [SMILES: CC(C)CNCc1cc(C(=O)Nc2cc(-c3ccc(C#N)cc3C(=O)N3CC(F)(F)C3)cc(OC(C)C)n2)c(=O)n(C)c1] |
| 212 | 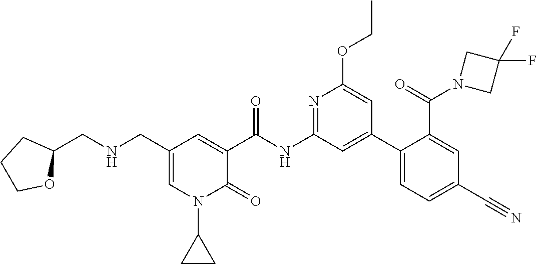 [SMILES: CCOc1cc(-c2ccc(C#N)cc2C(=O)N2CC(F)(F)C2)cc(NC(=O)c2cc(CNC[C@@H]3CCCO3)cn(C3CC3)c2=O)n1] |
| 213 | 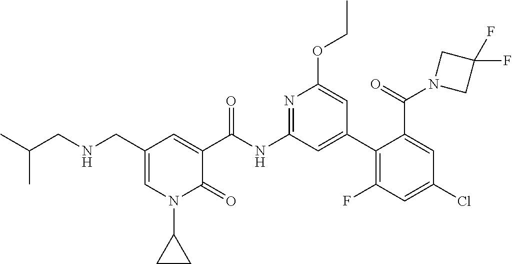 [SMILES: CCOc1cc(-c2c(F)cc(Cl)cc2C(=O)N2CC(F)(F)C2)cc(NC(=O)c2cc(CNCC(C)C)cn(C3CC3)c2=O)n1] |
| 214 | 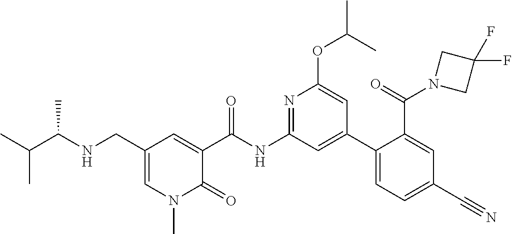 [SMILES: CC(C)Oc1cc(-c2ccc(C#N)cc2C(=O)N2CC(F)(F)C2)cc(NC(=O)c2cc(CN[C@@H](C)C(C)C)cn(C)c2=O)n1] |
| 215 | 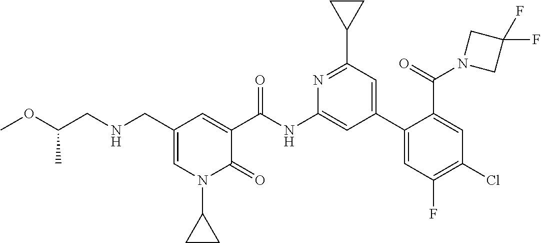 [SMILES: CO[C@@H](C)CNCc1cc(C(=O)Nc2cc(-c3cc(F)c(Cl)cc3C(=O)N3CC(F)(F)C3)cc(C3CC3)n2)c(=O)n(C2CC2)c1] |
| 216 | 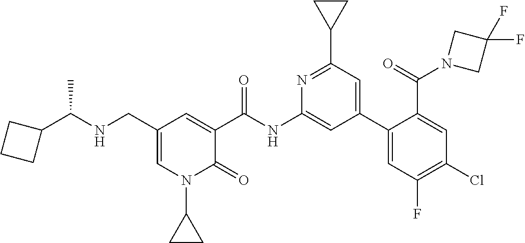 [SMILES: C[C@H](NCc1cc(C(=O)Nc2cc(-c3cc(F)c(Cl)cc3C(=O)N3CC(F)(F)C3)cc(C3CC3)n2)c(=O)n(C2CC2)c1)C1CCC1] |
| 217 | 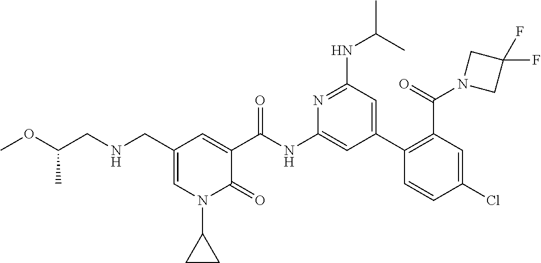 [SMILES: CO[C@@H](C)CNCc1cc(C(=O)Nc2cc(-c3ccc(Cl)cc3C(=O)N3CC(F)(F)C3)cc(NC(C)C)n2)c(=O)n(C2CC2)c1] |
| 218 | 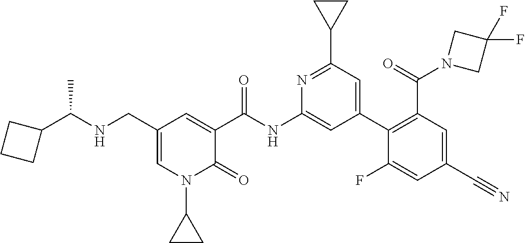 [SMILES: C[C@H](NCc1cc(C(=O)Nc2cc(-c3c(F)cc(C#N)cc3C(=O)N3CC(F)(F)C3)cc(C3CC3)n2)c(=O)n(C2CC2)c1)C1CCC1] |
| 219 | 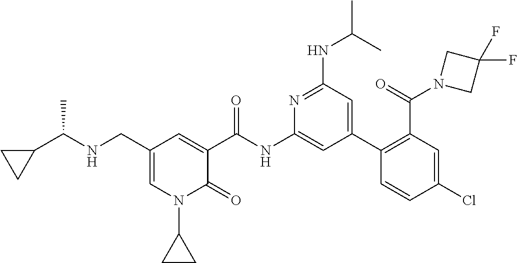 [SMILES: CC(C)Nc1cc(-c2ccc(Cl)cc2C(=O)N2CC(F)(F)C2)cc(NC(=O)c2cc(CN[C@@H](C)C3CC3)cn(C3CC3)c2=O)n1] |
| 220 | 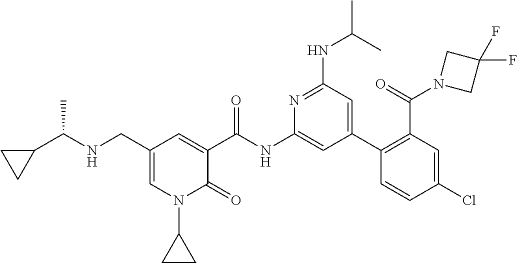 [SMILES: CC(C)Nc1cc(-c2ccc(Cl)cc2C(=O)N2CC(F)(F)C2)cc(NC(=O)c2cc(CN[C@@H](C)C3CC3)cn(C3CC3)c2=O)n1] |
| 221 | 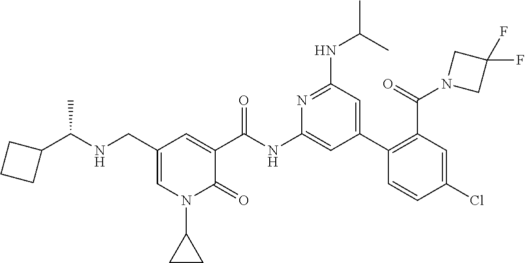 [SMILES: CC(C)Nc1cc(-c2ccc(Cl)cc2C(=O)N2CC(F)(F)C2)cc(NC(=O)c2cc(CN[C@@H](C)C3CCC3)cn(C3CC3)c2=O)n1] |
| 222 | 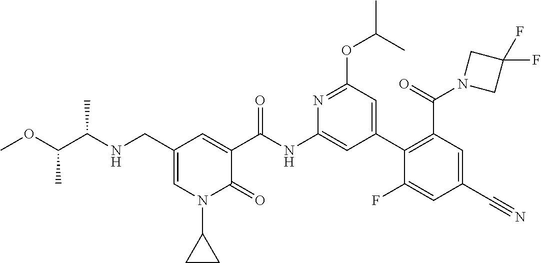 [SMILES: CO[C@@H](C)[C@H](C)NCc1cc(C(=O)Nc2cc(-c3c(F)cc(C#N)cc3C(=O)N3CC(F)(F)C3)cc(OC(C)C)n2)c(=O)n(C2CC2)c1] |
| 223 | 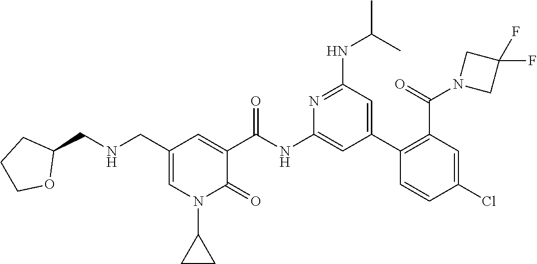 [SMILES: CC(C)Nc1cc(-c2ccc(Cl)cc2C(=O)N2CC(F)(F)C2)cc(NC(=O)c2cc(CNC[C@@H]3CCCO3)cn(C3CC3)c2=O)n1] |
| 224 | 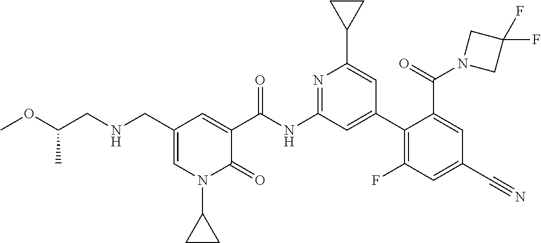 [SMILES: CO[C@@H](C)CNCc1cc(C(=O)Nc2cc(-c3c(F)cc(C#N)cc3C(=O)N3CC(F)(F)C3)cc(C3CC3)n2)c(=O)n(C2CC2)c1] |
| 225 | 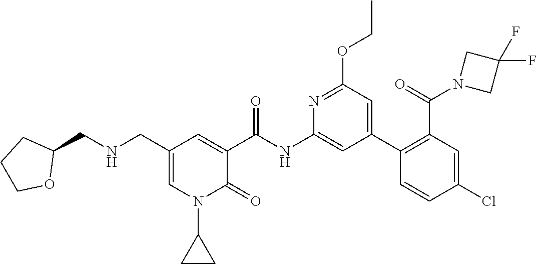 [SMILES: CCOc1cc(-c2ccc(Cl)cc2C(=O)N2CC(F)(F)C2)cc(NC(=O)c2cc(CNC[C@@H]3CCCO3)cn(C3CC3)c2=O)n1] |
| 226 | 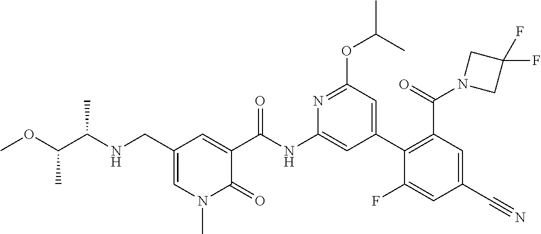 [SMILES: CO[C@@H](C)[C@H](C)NCc1cc(C(=O)Nc2cc(-c3c(F)cc(C#N)cc3C(=O)N3CC(F)(F)C3)cc(OC(C)C)n2)c(=O)n(C)c1] |
| 227 | 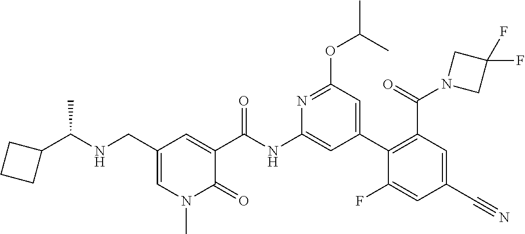 [SMILES: CC(C)Oc1cc(-c2c(F)cc(C#N)cc2C(=O)N2CC(F)(F)C2)cc(NC(=O)c2cc(CN[C@@H](C)C3CCC3)cn(C)c2=O)n1] |
| 228 | 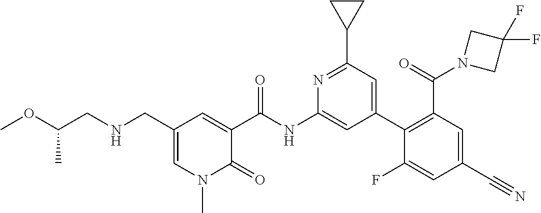 [SMILES: CO[C@@H](C)CNCc1cc(C(=O)Nc2cc(-c3c(F)cc(C#N)cc3C(=O)N3CC(F)(F)C3)cc(C3CC3)n2)c(=O)n(C)c1] |
| 229 | 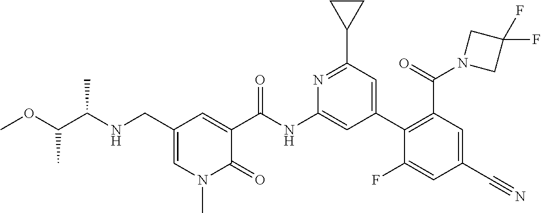 |
| 230 | 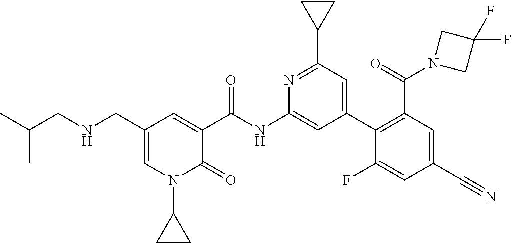 [SMILES: CC(C)CNCc1cc(C(=O)Nc2cc(-c3c(F)cc(C#N)cc3C(=O)N3CC(F)(F)C3)cc(C3CC3)n2)c(=O)n(C2CC2)c1] |
| 231 | 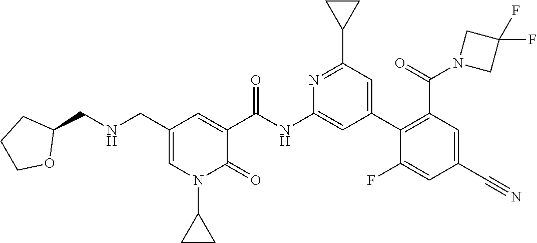 [SMILES: N#Cc1cc(F)c(-c2cc(NC(=O)c3cc(CNC[C@@H]4CCCO4)cn(C4CC4)c3=O)nc(C3CC3)c2)c(C(=O)N2CC(F)(F)C2)c1] |
| 232 | 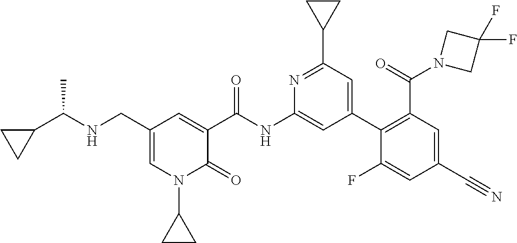 [SMILES: C[C@H](NCc1cc(C(=O)Nc2cc(-c3c(F)cc(C#N)cc3C(=O)N3CC(F)(F)C3)cc(C3CC3)n2)c(=O)n(C2CC2)c1)C1CC1] |
| 233 | 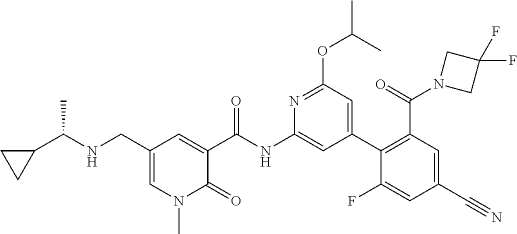 [SMILES: CC(C)Oc1cc(-c2c(F)cc(C#N)cc2C(=O)N2CC(F)(F)C2)cc(NC(=O)c2cc(CN[C@@H](C)C3CC3)cn(C)c2=O)n1] |
| 234 | 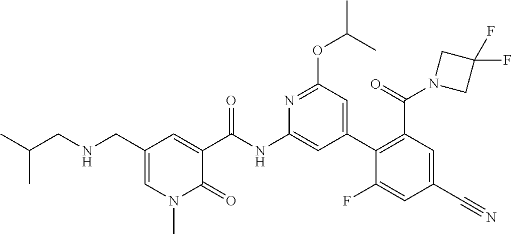 [SMILES: CC(C)CNCc1cc(C(=O)Nc2cc(-c3c(F)cc(C#N)cc3C(=O)N3CC(F)(F)C3)cc(OC(C)C)n2)c(=O)n(C)c1] |
| 235 | 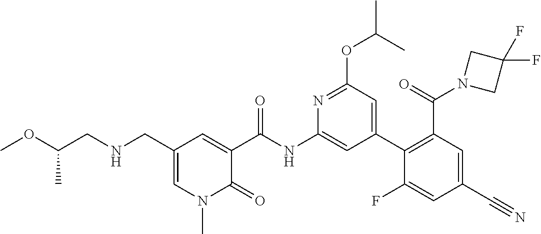 [SMILES: CO[C@@H](C)CNCc1cc(C(=O)Nc2cc(-c3c(F)cc(C#N)cc3C(=O)N3CC(F)(F)C3)cc(OC(C)C)n2)c(=O)n(C)c1] |
| 236 | 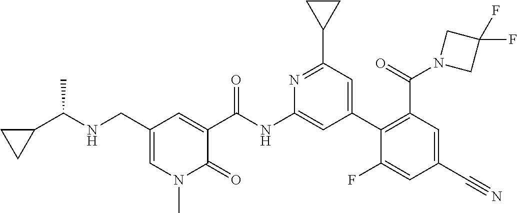 [SMILES: C[C@H](NCc1cc(C(=O)Nc2cc(-c3c(F)cc(C#N)cc3C(=O)N3CC(F)(F)C3)cc(C3CC3)n2)c(=O)n(C)c1)C1CC1] |
| 237 | 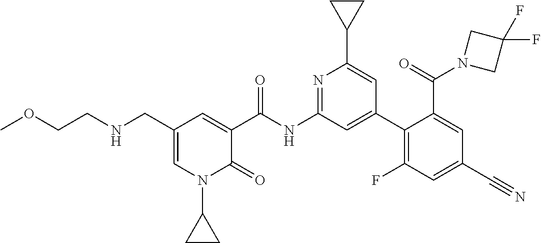 [SMILES: COCCNCc1cc(C(=O)Nc2cc(-c3c(F)cc(C#N)cc3C(=O)N3CC(F)(F)C3)cc(C3CC3)n2)c(=O)n(C2CC2)c1] |
| 238 | 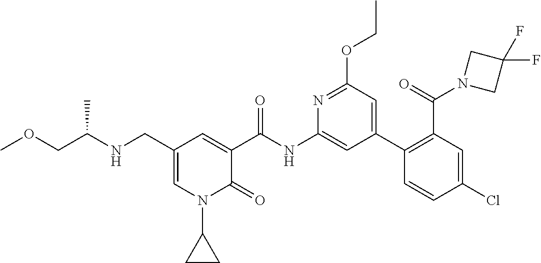 [SMILES: CCOc1cc(-c2ccc(Cl)cc2C(=O)N2CC(F)(F)C2)cc(NC(=O)c2cc(CN[C@@H](C)COC)cn(C3CC3)c2=O)n1] |
| 239 | 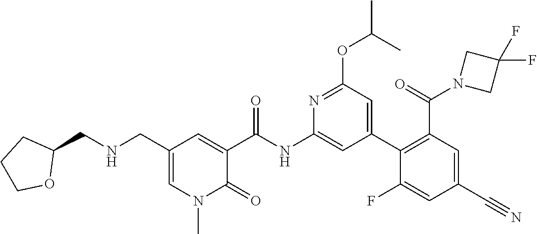 [SMILES: CC(C)Oc1cc(-c2c(F)cc(C#N)cc2C(=O)N2CC(F)(F)C2)cc(NC(=O)c2cc(CNC[C@@H]3CCCO3)cn(C)c2=O)n1] |
| 240 | 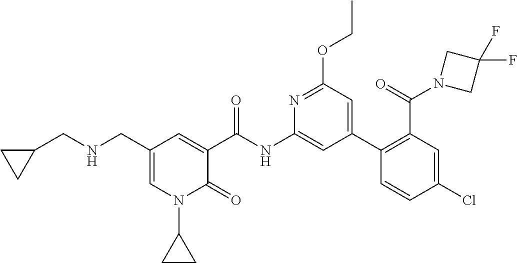 [SMILES: CCOc1cc(-c2ccc(Cl)cc2C(=O)N2CC(F)(F)C2)cc(NC(=O)c2cc(CNCC3CC3)cn(C3CC3)c2=O)n1] |
| 241 | 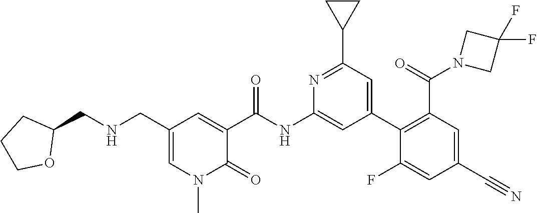 [SMILES: Cn1cc(CNC[C@@H]2CCCO2)cc(C(=O)Nc2cc(-c3c(F)cc(C#N)cc3C(=O)N3CC(F)(F)C3)cc(C3CC3)n2)c1=O] |
| 242 | 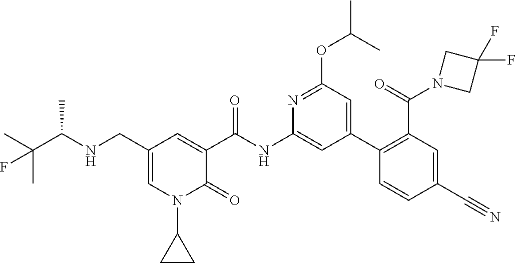 [SMILES: CC(C)Oc1cc(-c2ccc(C#N)cc2C(=O)N2CC(F)(F)C2)cc(NC(=O)c2cc(CN[C@@H](C)C(C)(C)F)cn(C3CC3)c2=O)n1] |
| 243 | 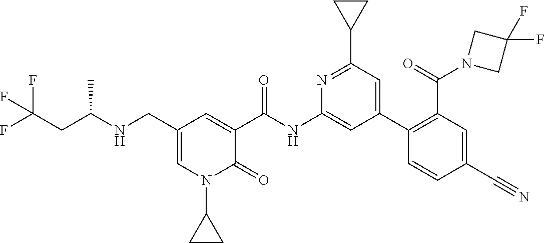 [SMILES: C[C@@H](CC(F)(F)F)NCc1cc(C(=O)Nc2cc(-c3ccc(C#N)cc3C(=O)N3CC(F)(F)C3)cc(C3CC3)n2)c(=O)n(C2CC2)c1] |
| 244 | 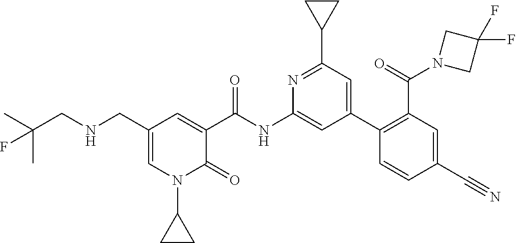 [SMILES: CC(C)(F)CNCc1cc(C(=O)Nc2cc(-c3ccc(C#N)cc3C(=O)N3CC(F)(F)C3)cc(C3CC3)n2)c(=O)n(C2CC2)c1] |
| 245 | 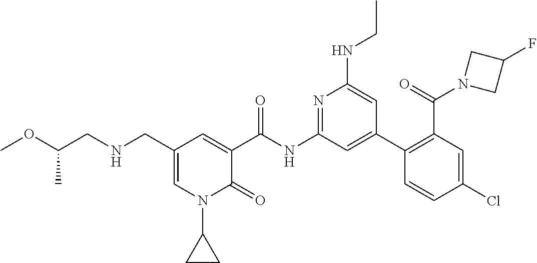 [SMILES: CCNc1cc(-c2ccc(Cl)cc2C(=O)N2CC(F)C2)cc(NC(=O)c2cc(CNC[C@H](C)OC)cn(C3CC3)c2=O)n1] |
| 246 | 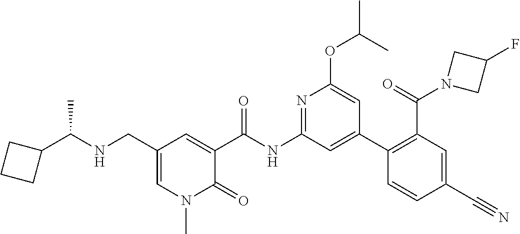 [SMILES: CC(C)Oc1cc(-c2ccc(C#N)cc2C(=O)N2CC(F)C2)cc(NC(=O)c2cc(CN[C@@H](C)C3CCC3)cn(C)c2=O)n1] |
| 247 | 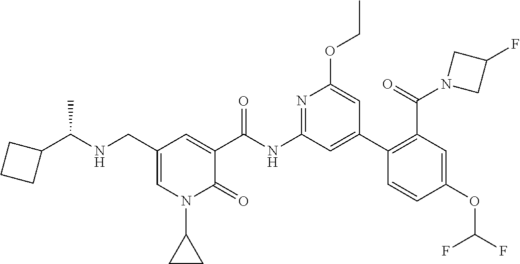 [SMILES: CCOc1cc(-c2ccc(OC(F)F)cc2C(=O)N2CC(F)C2)cc(NC(=O)c2cc(CN[C@@H](C)C3CCC3)cn(C3CC3)c2=O)n1] |
| 248 | 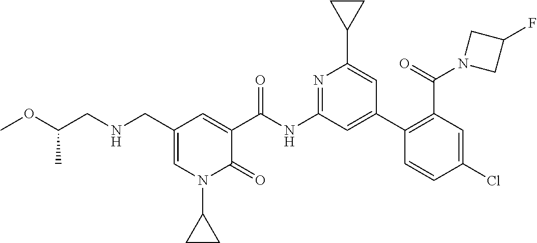 [SMILES: CO[C@@H](C)CNCc1cc(C(=O)Nc2cc(-c3ccc(Cl)cc3C(=O)N3CC(F)C3)cc(C3CC3)n2)c(=O)n(C2CC2)c1] |
| 249 | 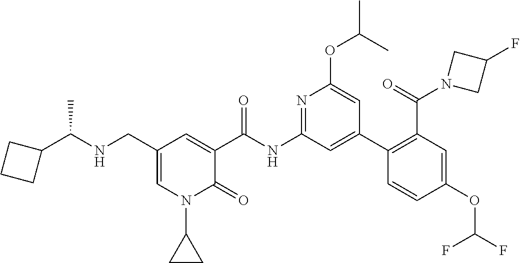 [SMILES: CC(C)Oc1cc(-c2ccc(OC(F)F)cc2C(=O)N2CC(F)C2)cc(NC(=O)c2cc(CN[C@@H](C)C3CCC3)cn(C3CC3)c2=O)n1] |
| 250 | 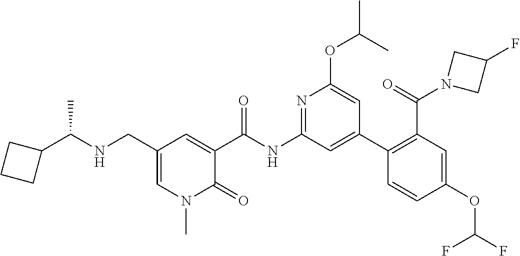 [SMILES: CC(C)Oc1cc(-c2ccc(OC(F)F)cc2C(=O)N2CC(F)C2)cc(NC(=O)c2cc(CN[C@@H](C)C3CCC3)cn(C)c2=O)n1] |
| 251 | 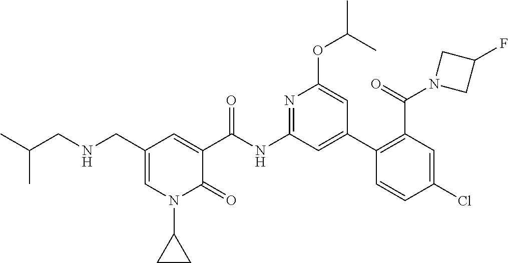 [SMILES: CC(C)CNCc1cc(C(=O)Nc2cc(-c3ccc(Cl)cc3C(=O)N3CC(F)C3)cc(OC(C)C)n2)c(=O)n(C2CC2)c1] |
| 252 | 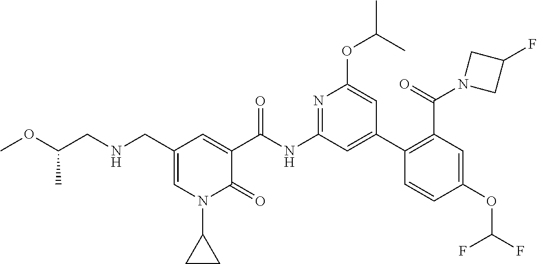 [SMILES: CO[C@@H](C)CNCc1cc(C(=O)Nc2cc(-c3ccc(OC(F)F)cc3C(=O)N3CC(F)C3)cc(OC(C)C)n2)c(=O)n(C2CC2)c1] |
| 253 | 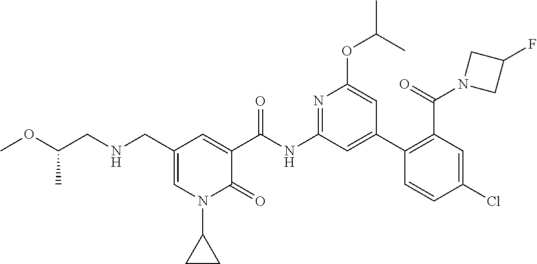 [SMILES: CO[C@@H](C)CNCc1cc(C(=O)Nc2cc(-c3ccc(Cl)cc3C(=O)N3CC(F)C3)cc(OC(C)C)n2)c(=O)n(C2CC2)c1] |
| 254 | 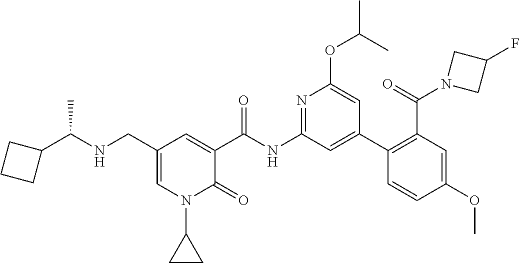 [SMILES: COc1ccc(-c2cc(NC(=O)c3cc(CN[C@@H](C)C4CCC4)cn(C4CC4)c3=O)nc(OC(C)C)c2)c(C(=O)N2CC(F)C2)c1] |
| 255 | 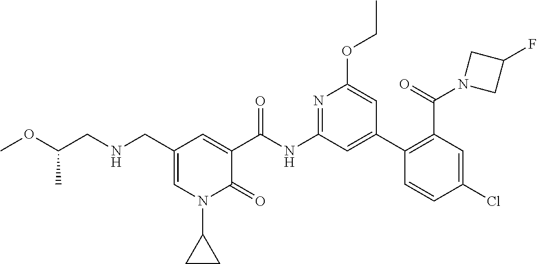 [SMILES: CCOc1cc(-c2ccc(Cl)cc2C(=O)N2CC(F)C2)cc(NC(=O)c2cc(CNC[C@H](C)OC)cn(C3CC3)c2=O)n1] |
| 256 | 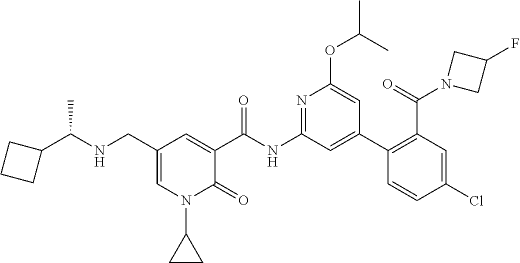 [SMILES: CC(C)Oc1cc(-c2ccc(Cl)cc2C(=O)N2CC(F)C2)cc(NC(=O)c2cc(CN[C@@H](C)C3CCC3)cn(C3CC3)c2=O)n1] |
| 257 | 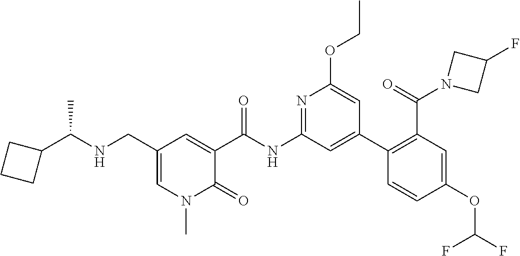 [SMILES: CCOc1cc(-c2ccc(OC(F)F)cc2C(=O)N2CC(F)C2)cc(NC(=O)c2cc(CN[C@@H](C)C3CCC3)cn(C)c2=O)n1] |
| 258 | 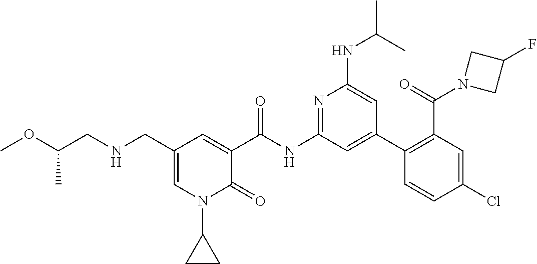 [SMILES: CO[C@@H](C)CNCc1cc(C(=O)Nc2cc(-c3ccc(Cl)cc3C(=O)N3CC(F)C3)cc(NC(C)C)n2)c(=O)n(C2CC2)c1] |
| 259 | 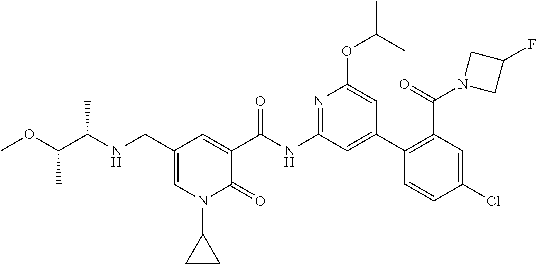 [SMILES: CO[C@@H](C)[C@H](C)NCc1cc(C(=O)Nc2cc(-c3ccc(Cl)cc3C(=O)N3CC(F)C3)cc(OC(C)C)n2)c(=O)n(C2CC2)c1] |
| 260 | 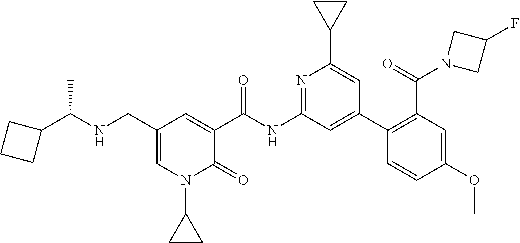 [SMILES: COc1ccc(-c2cc(NC(=O)c3cc(CN[C@@H](C)C4CCC4)cn(C4CC4)c3=O)nc(C3CC3)c2)c(C(=O)N2CC(F)C2)c1] |
| 261 | 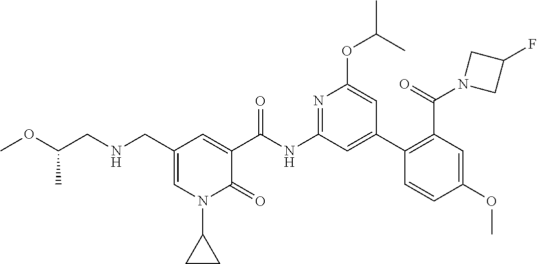 [SMILES: COc1ccc(-c2cc(NC(=O)c3cc(CNC[C@H](C)OC)cn(C4CC4)c3=O)nc(OC(C)C)c2)c(C(=O)N2CC(F)C2)c1] |
| 262 | 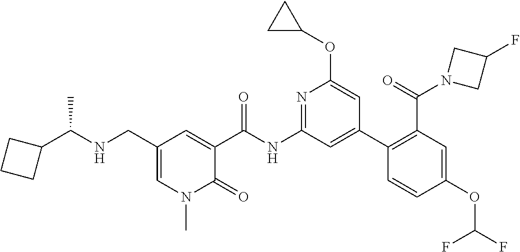 [SMILES: C[C@H](NCc1cc(C(=O)Nc2cc(-c3ccc(OC(F)F)cc3C(=O)N3CC(F)C3)cc(OC3CC3)n2)c(=O)n(C)c1)C1CCC1] |
| 263 | 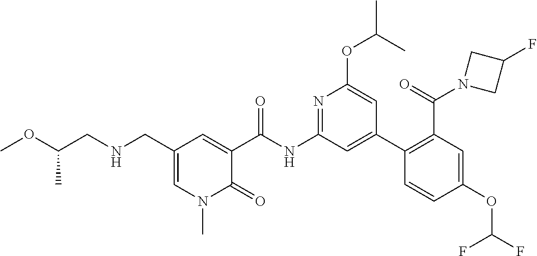 [SMILES: CO[C@@H](C)CNCc1cc(C(=O)Nc2cc(-c3ccc(OC(F)F)cc3C(=O)N3CC(F)C3)cc(OC(C)C)n2)c(=O)n(C)c1] |
| 264 | 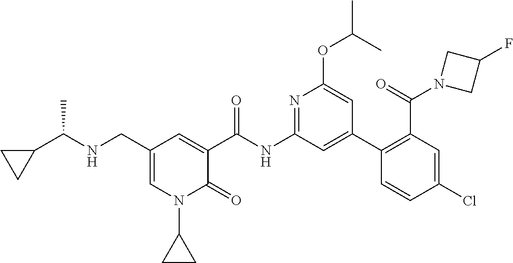 [SMILES: CC(C)Oc1cc(-c2ccc(Cl)cc2C(=O)N2CC(F)C2)cc(NC(=O)c2cc(CN[C@@H](C)C3CC3)cn(C3CC3)c2=O)n1] |
| 265 | 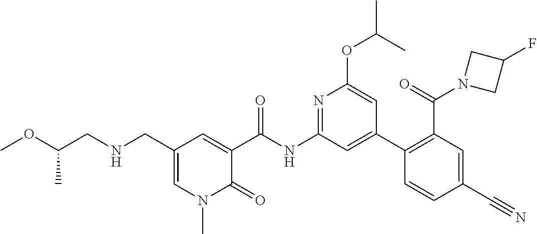 [SMILES: CO[C@@H](C)CNCc1cc(C(=O)Nc2cc(-c3ccc(C#N)cc3C(=O)N3CC(F)C3)cc(OC(C)C)n2)c(=O)n(C)c1] |
| 266 | 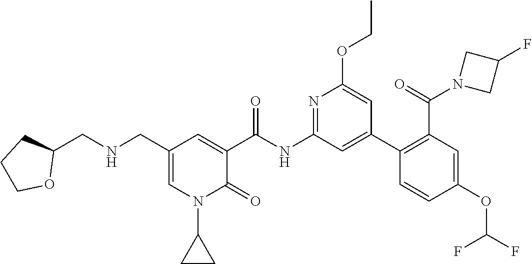 [SMILES: CCOc1cc(-c2ccc(OC(F)F)cc2C(=O)N2CC(F)C2)cc(NC(=O)c2cc(CNC[C@@H]3CCCO3)cn(C3CC3)c2=O)n1] |
| 267 | 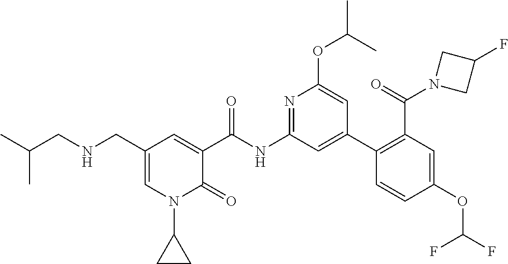 [SMILES: CC(C)CNCc1cc(C(=O)Nc2cc(-c3ccc(OC(F)F)cc3C(=O)N3CC(F)C3)cc(OC(C)C)n2)c(=O)n(C2CC2)c1] |
| 268 | 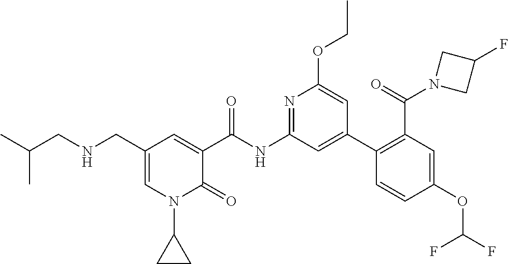 [SMILES: CCOc1cc(-c2ccc(OC(F)F)cc2C(=O)N2CC(F)C2)cc(NC(=O)c2cc(CNCC(C)C)cn(C3CC3)c2=O)n1] |
| 269 | 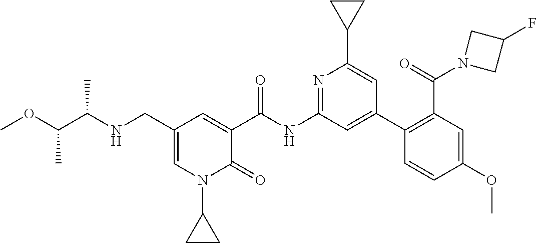 [SMILES: COc1ccc(-c2cc(NC(=O)c3cc(CN[C@@H](C)[C@H](C)OC)cn(C4CC4)c3=O)nc(C3CC3)c2)c(C(=O)N2CC(F)C2)c1] |
| 270 | 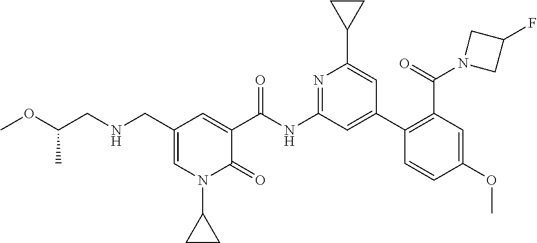 [SMILES: COc1ccc(-c2cc(NC(=O)c3cc(CNC[C@H](C)OC)cn(C4CC4)c3=O)nc(C3CC3)c2)c(C(=O)N2CC(F)C2)c1] |
| 271 | 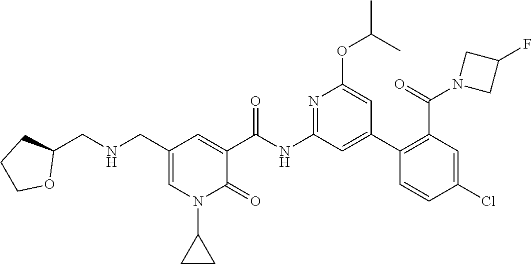 [SMILES: CC(C)Oc1cc(-c2ccc(Cl)cc2C(=O)N2CC(F)C2)cc(NC(=O)c2cc(CNC[C@@H]3CCCO3)cn(C3CC3)c2=O)n1] |
| 272 | 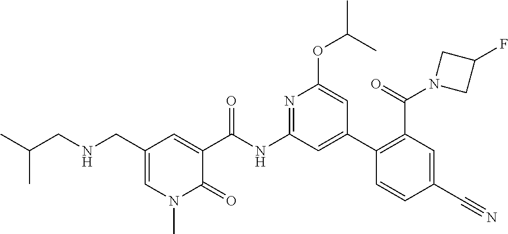 [SMILES: CC(C)CNCc1cc(C(=O)Nc2cc(-c3ccc(C#N)cc3C(=O)N3CC(F)C3)cc(OC(C)C)n2)c(=O)n(C)c1] |
| 273 | 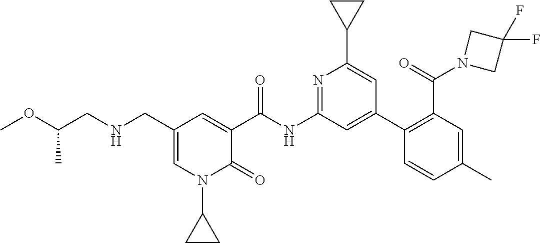 [SMILES: CO[C@@H](C)CNCc1cc(C(=O)Nc2cc(-c3ccc(C)cc3C(=O)N3CC(F)(F)C3)cc(C3CC3)n2)c(=O)n(C2CC2)c1] |
| 274 | 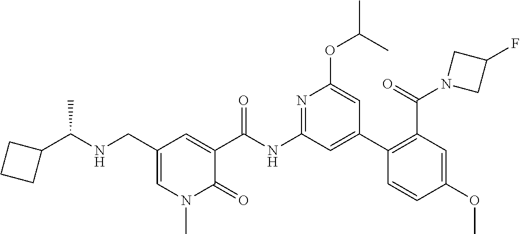 [SMILES: COc1ccc(-c2cc(NC(=O)c3cc(CN[C@@H](C)C4CCC4)cn(C)c3=O)nc(OC(C)C)c2)c(C(=O)N2CC(F)C2)c1] |
| 275 | 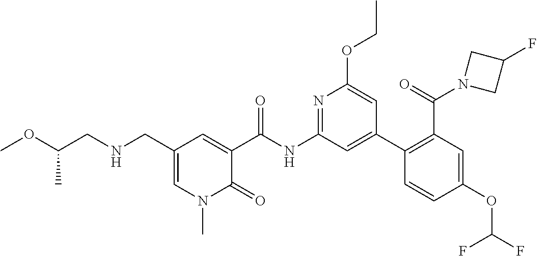 [SMILES: CCOc1cc(-c2ccc(OC(F)F)cc2C(=O)N2CC(F)C2)cc(NC(=O)c2cc(CNC[C@H](C)OC)cn(C)c2=O)n1] |
| 276 | 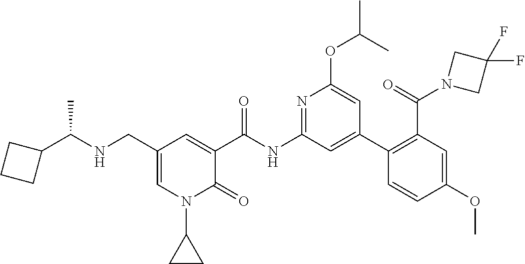 [SMILES: COc1ccc(-c2cc(NC(=O)c3cc(CN[C@@H](C)C4CCC4)cn(C4CC4)c3=O)nc(OC(C)C)c2)c(C(=O)N2CC(F)(F)C2)c1] |
| 277 | 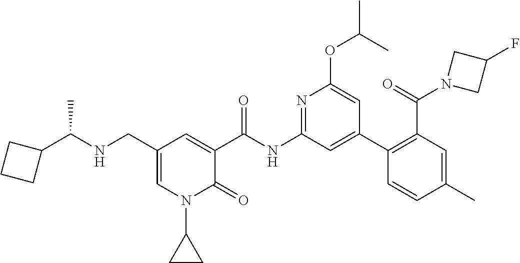 [SMILES: Cc1ccc(-c2cc(NC(=O)c3cc(CN[C@@H](C)C4CCC4)cn(C4CC4)c3=O)nc(OC(C)C)c2)c(C(=O)N2CC(F)C2)c1] |
| 278 | 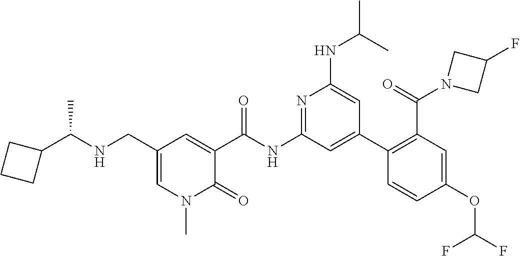 [SMILES: CC(C)Nc1cc(-c2ccc(OC(F)F)cc2C(=O)N2CC(F)C2)cc(NC(=O)c2cc(CN[C@@H](C)C3CCC3)cn(C)c2=O)n1] |
| 279 | 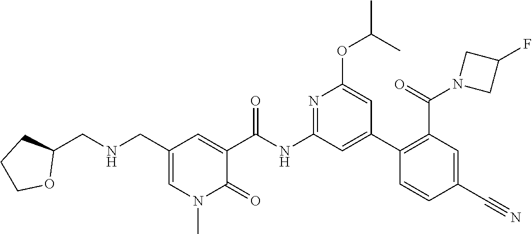 [SMILES: CC(C)Oc1cc(-c2ccc(C#N)cc2C(=O)N2CC(F)C2)cc(NC(=O)c2cc(CNC[C@@H]3CCCO3)cn(C)c2=O)n1] |
| 280 | 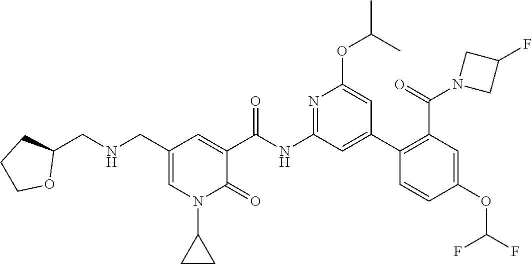 [SMILES: CC(C)Oc1cc(-c2ccc(OC(F)F)cc2C(=O)N2CC(F)C2)cc(NC(=O)c2cc(CNC[C@@H]3CCCO3)cn(C3CC3)c2=O)n1] |
| 281 | 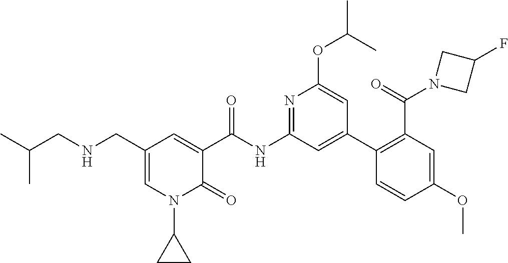 [SMILES: COc1ccc(-c2cc(NC(=O)c3cc(CNCC(C)C)cn(C4CC4)c3=O)nc(OC(C)C)c2)c(C(=O)N2CC(F)C2)c1] |
| 282 | 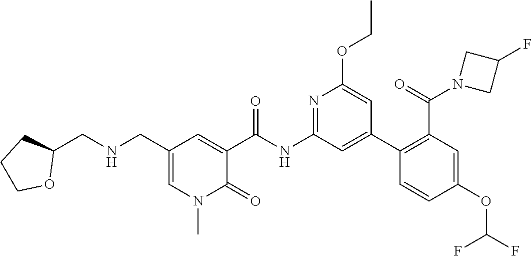 [SMILES: CCOc1cc(-c2ccc(OC(F)F)cc2C(=O)N2CC(F)C2)cc(NC(=O)c2cc(CNC[C@@H]3CCCO3)cn(C)c2=O)n1] |
| 283 | 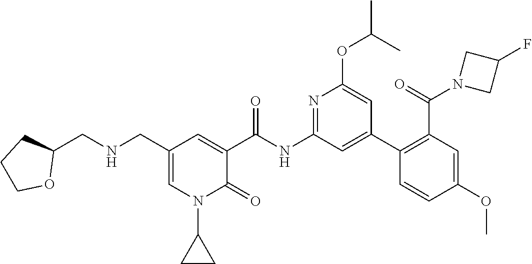 [SMILES: COc1ccc(-c2cc(NC(=O)c3cc(CNC[C@@H]4CCCO4)cn(C4CC4)c3=O)nc(OC(C)C)c2)c(C(=O)N2CC(F)C2)c1] |
| 284 | 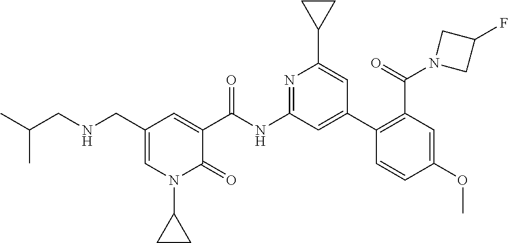 [SMILES: COc1ccc(-c2cc(NC(=O)c3cc(CNCC(C)C)cn(C4CC4)c3=O)nc(C3CC3)c2)c(C(=O)N2CC(F)C2)c1] |
| 285 | 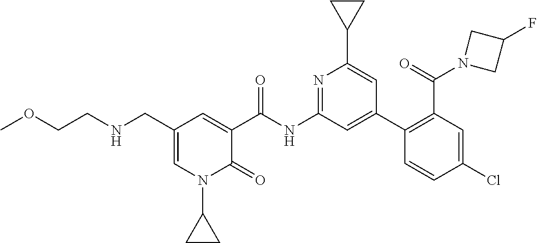 [SMILES: COCCNCc1cc(C(=O)Nc2cc(-c3ccc(Cl)cc3C(=O)N3CC(F)C3)cc(C3CC3)n2)c(=O)n(C2CC2)c1] |
| 286 | 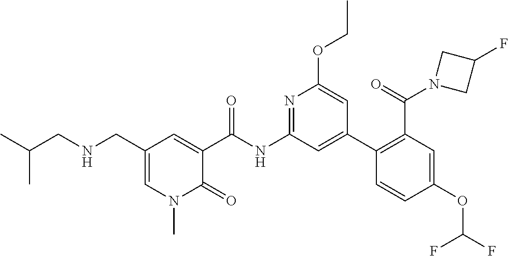 [SMILES: CCOc1cc(-c2ccc(OC(F)F)cc2C(=O)N2CC(F)C2)cc(NC(=O)c2cc(CNCC(C)C)cn(C)c2=O)n1] |
| 287 | 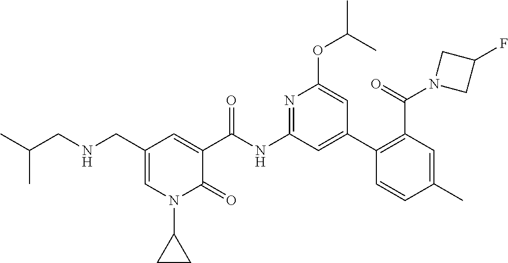 [SMILES: Cc1ccc(-c2cc(NC(=O)c3cc(CNCC(C)C)cn(C4CC4)c3=O)nc(OC(C)C)c2)c(C(=O)N2CC(F)C2)c1] |
| 288 | 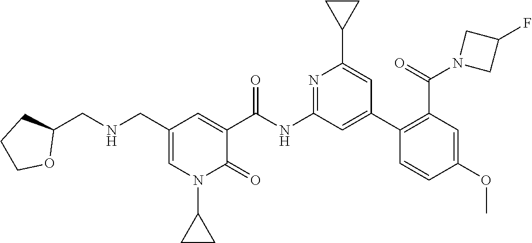 [SMILES: COc1ccc(-c2cc(NC(=O)c3cc(CNC[C@@H]4CCCO4)cn(C4CC4)c3=O)nc(C3CC3)c2)c(C(=O)N2CC(F)C2)c1] |
| 289 | 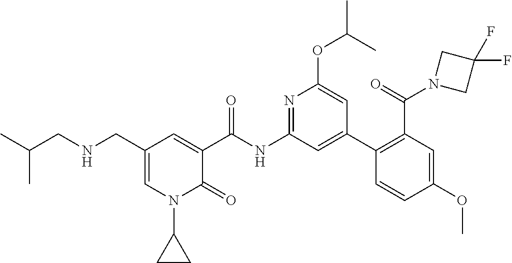 [SMILES: COc1ccc(-c2cc(NC(=O)c3cc(CNCC(C)C)cn(C4CC4)c3=O)nc(OC(C)C)c2)c(C(=O)N2CC(F)(F)C2)c1] |
| 290 | 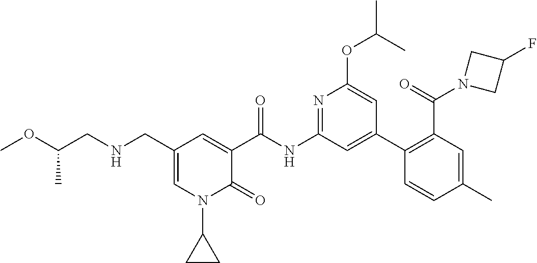 [SMILES: CO[C@@H](C)CNCc1cc(C(=O)Nc2cc(-c3ccc(C)cc3C(=O)N3CC(F)C3)cc(OC(C)C)n2)c(=O)n(C2CC2)c1] |
| 291 | 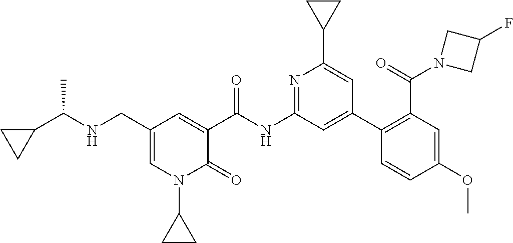 [SMILES: COc1ccc(-c2cc(NC(=O)c3cc(CN[C@@H](C)C4CC4)cn(C4CC4)c3=O)nc(C3CC3)c2)c(C(=O)N2CC(F)C2)c1] |
| 292 | 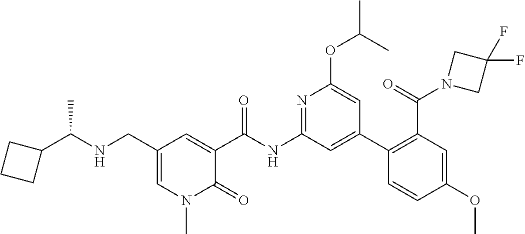 [SMILES: COc1ccc(-c2cc(NC(=O)c3cc(CN[C@@H](C)C4CCC4)cn(C)c3=O)nc(OC(C)C)c2)c(C(=O)N2CC(F)(F)C2)c1] |
| 293 | 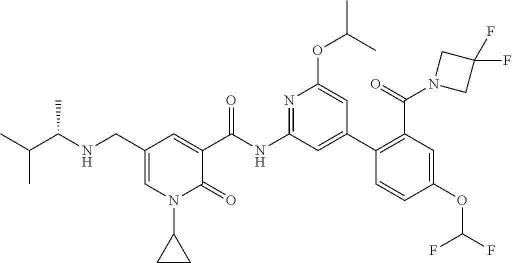 [SMILES: CC(C)Oc1cc(-c2ccc(OC(F)F)cc2C(=O)N2CC(F)(F)C2)cc(NC(=O)c2cc(CN[C@@H](C)C(C)C)cn(C3CC3)c2=O)n1] |
| 294 | 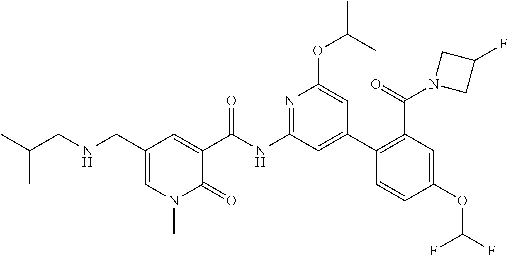 [SMILES: CC(C)CNCc1cc(C(=O)Nc2cc(-c3ccc(OC(F)F)cc3C(=O)N3CC(F)C3)cc(OC(C)C)n2)c(=O)n(C)c1] |
| 295 | 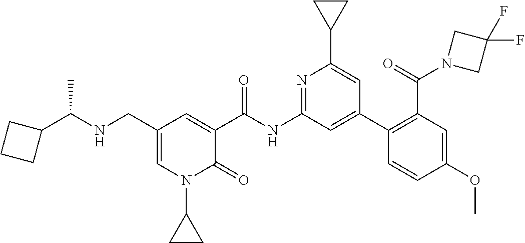 [SMILES: COc1ccc(-c2cc(NC(=O)c3cc(CN[C@@H](C)C4CCC4)cn(C4CC4)c3=O)nc(C3CC3)c2)c(C(=O)N2CC(F)(F)C2)c1] |
| 296 | 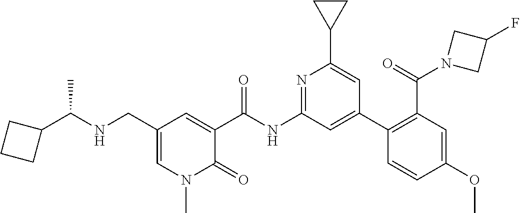 [SMILES: COc1ccc(-c2cc(NC(=O)c3cc(CN[C@@H](C)C4CCC4)cn(C)c3=O)nc(C3CC3)c2)c(C(=O)N2CC(F)C2)c1] |
| 297 | 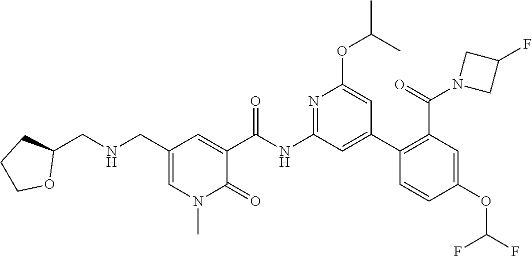 [SMILES: CC(C)Oc1cc(-c2ccc(OC(F)F)cc2C(=O)N2CC(F)C2)cc(NC(=O)c2cc(CNC[C@@H]3CCCO3)cn(C)c2=O)n1] |
| 298 | 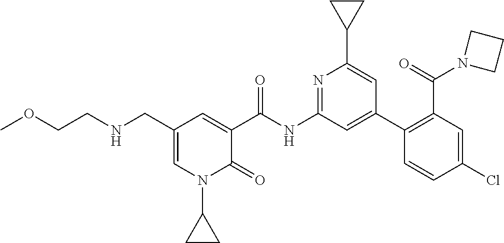 [SMILES: COCCNCc1cc(C(=O)Nc2cc(-c3ccc(Cl)cc3C(=O)N3CCC3)cc(C3CC3)n2)c(=O)n(C2CC2)c1] |
| 299 | 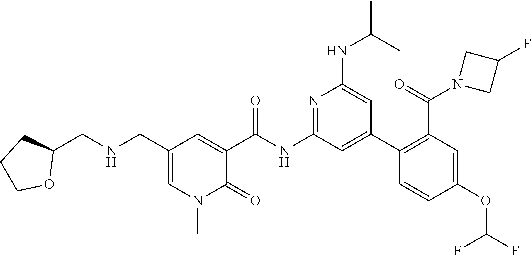 [SMILES: CC(C)Nc1cc(-c2ccc(OC(F)F)cc2C(=O)N2CC(F)C2)cc(NC(=O)c2cc(CNC[C@@H]3CCCO3)cn(C)c2=O)n1] |
| 300 | 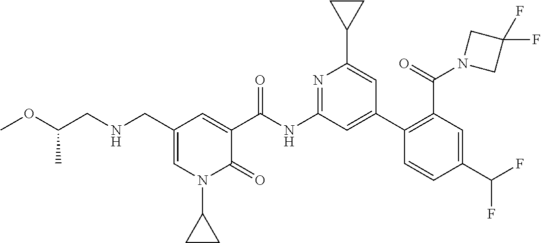 [SMILES: CO[C@@H](C)CNCc1cc(C(=O)Nc2cc(-c3ccc(C(F)F)cc3C(=O)N3CC(F)(F)C3)cc(C3CC3)n2)c(=O)n(C2CC2)c1] |
| 301 | 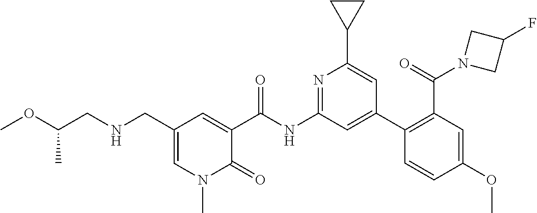 [SMILES: COc1ccc(-c2cc(NC(=O)c3cc(CNC[C@H](C)OC)cn(C)c3=O)nc(C3CC3)c2)c(C(=O)N2CC(F)C2)c1] |
| 302 | 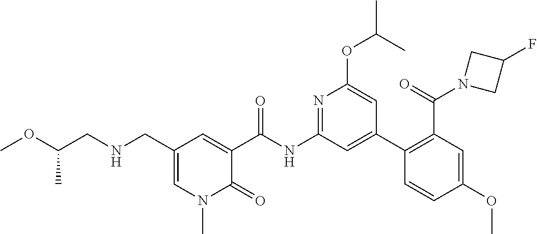 [SMILES: COc1ccc(-c2cc(NC(=O)c3cc(CNC[C@H](C)OC)cn(C)c3=O)nc(OC(C)C)c2)c(C(=O)N2CC(F)C2)c1] |
| 303 | 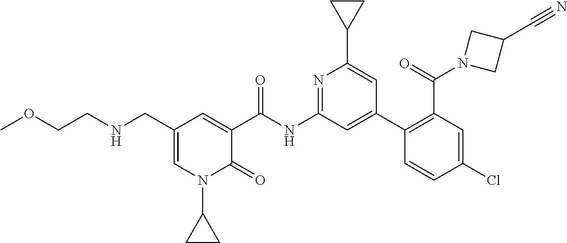 [SMILES: COCCNCc1cc(C(=O)Nc2cc(-c3ccc(Cl)cc3C(=O)N3CC(C#N)C3)cc(C3CC3)n2)c(=O)n(C2CC2)c1] |
| 304 | 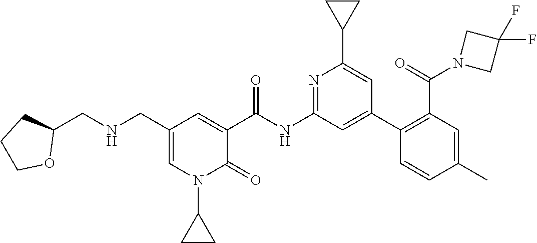 [SMILES: Cc1ccc(-c2cc(NC(=O)c3cc(CNC[C@@H]4CCCO4)cn(C4CC4)c3=O)nc(C3CC3)c2)c(C(=O)N2CC(F)(F)C2)c1] |
| 305 | 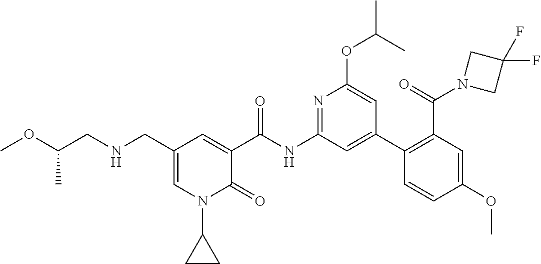 [SMILES: COc1ccc(-c2cc(NC(=O)c3cc(CNC[C@H](C)OC)cn(C4CC4)c3=O)nc(OC(C)C)c2)c(C(=O)N2CC(F)(F)C2)c1] |
| 306 | 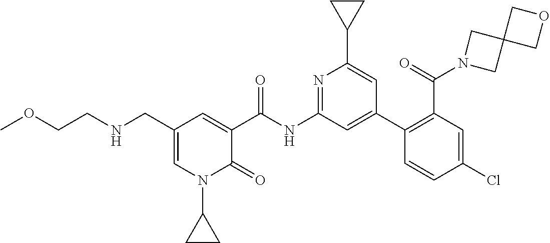 [SMILES: COCCNCc1cc(C(=O)Nc2cc(-c3ccc(Cl)cc3C(=O)N3CC4(COC4)C3)cc(C3CC3)n2)c(=O)n(C2CC2)c1] |
| 307 | 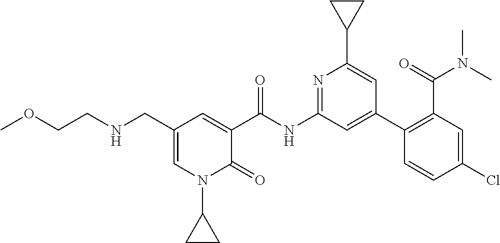 |
| 308 | 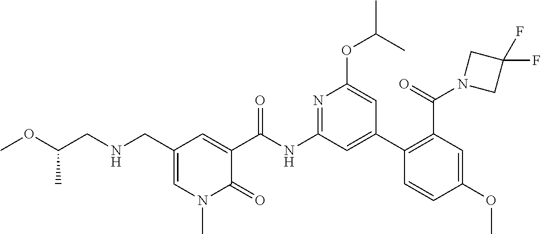 [SMILES: COc1ccc(-c2cc(NC(=O)c3cc(CNC[C@H](C)OC)cn(C)c3=O)nc(OC(C)C)c2)c(C(=O)N2CC(F)(F)C2)c1] |
| 309 | 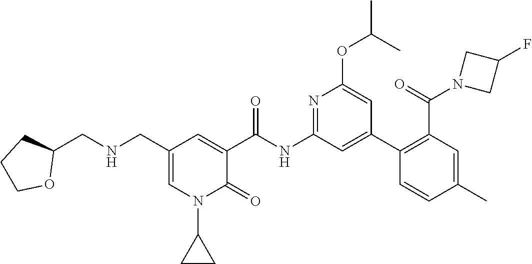 [SMILES: Cc1ccc(-c2cc(NC(=O)c3cc(CNC[C@@H]4CCCO4)cn(C4CC4)c3=O)nc(OC(C)C)c2)c(C(=O)N2CC(F)C2)c1] |
| 310 | 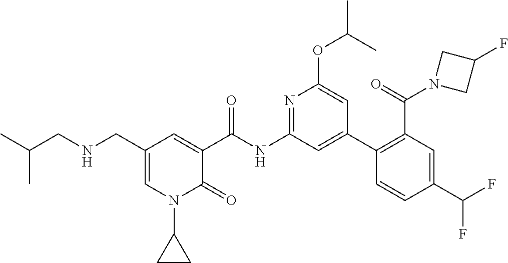 [SMILES: CC(C)CNCc1cc(C(=O)Nc2cc(-c3ccc(C(F)F)cc3C(=O)N3CC(F)C3)cc(OC(C)C)n2)c(=O)n(C2CC2)c1] |
| 311 | 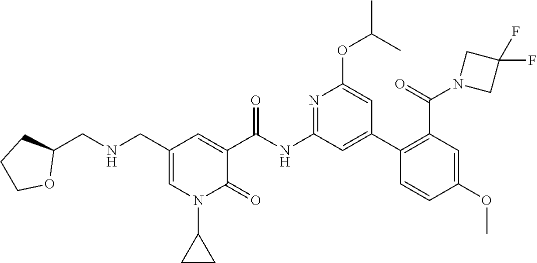 [SMILES: COc1ccc(-c2cc(NC(=O)c3cc(CNC[C@@H]4CCCO4)cn(C4CC4)c3=O)nc(OC(C)C)c2)c(C(=O)N2CC(F)(F)C2)c1] |
| 312 | 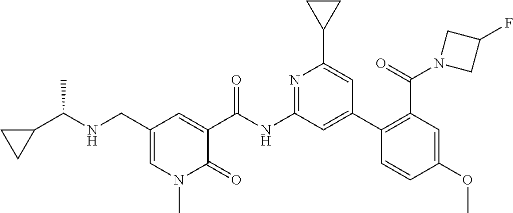 [SMILES: COc1ccc(-c2cc(NC(=O)c3cc(CN[C@@H](C)C4CC4)cn(C)c3=O)nc(C3CC3)c2)c(C(=O)N2CC(F)C2)c1] |
| 313 | 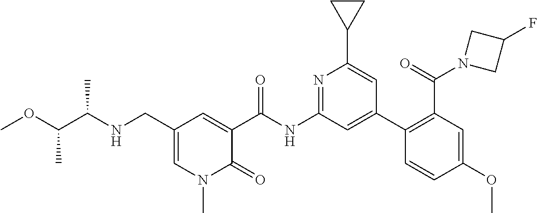 [SMILES: COc1ccc(-c2cc(NC(=O)c3cc(CN[C@@H](C)[C@H](C)OC)cn(C)c3=O)nc(C3CC3)c2)c(C(=O)N2CC(F)C2)c1] |
| 314 | 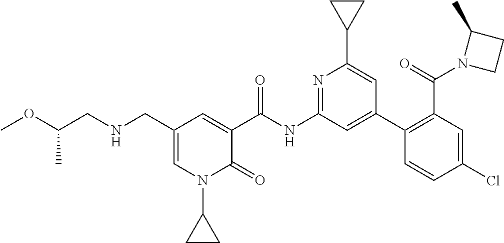 [SMILES: CO[C@@H](C)CNCc1cc(C(=O)Nc2cc(-c3ccc(Cl)cc3C(=O)N3CC[C@@H]3C)cc(C3CC3)n2)c(=O)n(C2CC2)c1] |
| 315 | 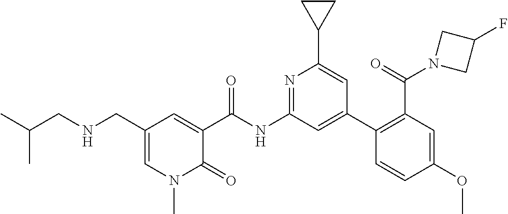 [SMILES: COc1ccc(-c2cc(NC(=O)c3cc(CNCC(C)C)cn(C)c3=O)nc(C3CC3)c2)c(C(=O)N2CC(F)C2)c1] |
| 316 | 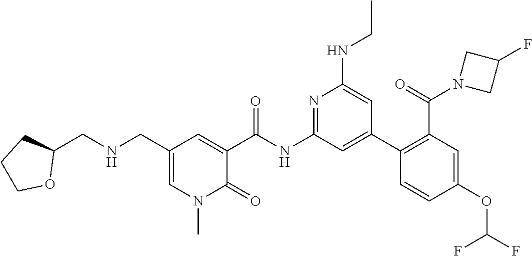 [SMILES: CCNc1cc(-c2ccc(OC(F)F)cc2C(=O)N2CC(F)C2)cc(NC(=O)c2cc(CNC[C@@H]3CCCO3)cn(C)c2=O)n1] |
| 317 | 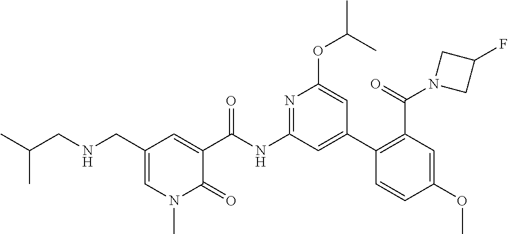 [SMILES: COc1ccc(-c2cc(NC(=O)c3cc(CNCC(C)C)cn(C)c3=O)nc(OC(C)C)c2)c(C(=O)N2CC(F)C2)c1] |
| 318 | 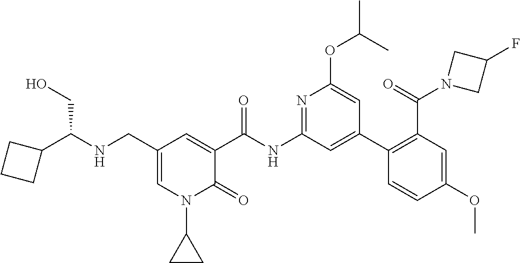 [SMILES: COc1ccc(-c2cc(NC(=O)c3cc(CN[C@@H](CO)C4CCC4)cn(C4CC4)c3=O)nc(OC(C)C)c2)c(C(=O)N2CC(F)C2)c1] |
| 319 | 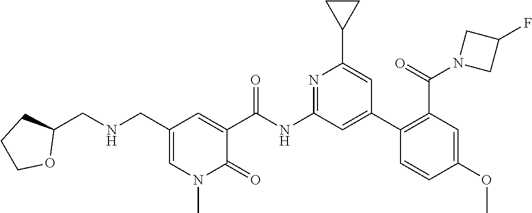 [SMILES: COc1ccc(-c2cc(NC(=O)c3cc(CNC[C@@H]4CCCO4)cn(C)c3=O)nc(C3CC3)c2)c(C(=O)N2CC(F)C2)c1] |
| 320 | 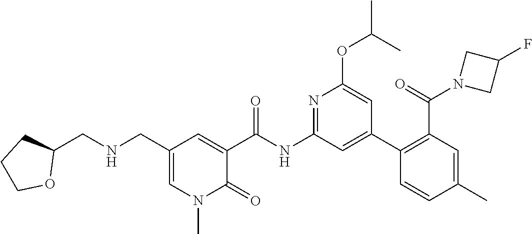 [SMILES: Cc1ccc(-c2cc(NC(=O)c3cc(CNC[C@@H]4CCCO4)cn(C)c3=O)nc(OC(C)C)c2)c(C(=O)N2CC(F)C2)c1] |
| 321 | 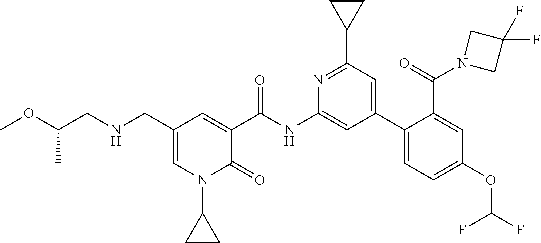 [SMILES: CO[C@@H](C)CNCc1cc(C(=O)Nc2cc(-c3ccc(OC(F)F)cc3C(=O)N3CC(F)(F)C3)cc(C3CC3)n2)c(=O)n(C2CC2)c1] |
| 322 | 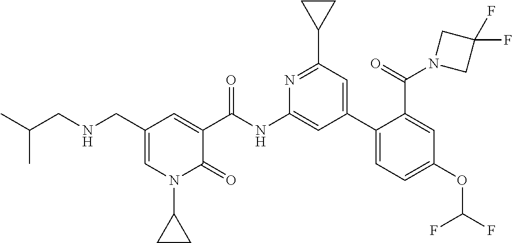 [SMILES: CC(C)CNCc1cc(C(=O)Nc2cc(-c3ccc(OC(F)F)cc3C(=O)N3CC(F)(F)C3)cc(C3CC3)n2)c(=O)n(C2CC2)c1] |
| 323 | 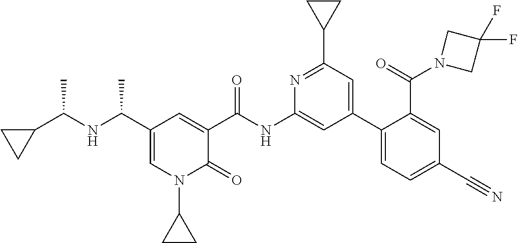 [SMILES: C[C@H](N[C@H](C)c1cc(C(=O)Nc2cc(-c3ccc(C#N)cc3C(=O)N3CC(F)(F)C3)cc(C3CC3)n2)c(=O)n(C2CC2)c1)C1CC1] |
| 324 | 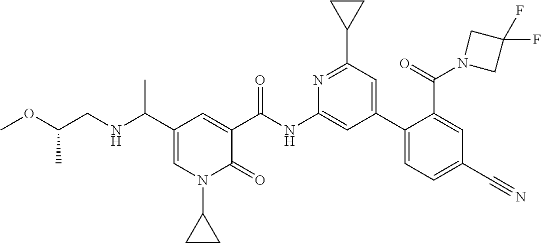 [SMILES: CO[C@@H](C)CNC(C)c1cc(C(=O)Nc2cc(-c3ccc(C#N)cc3C(=O)N3CC(F)(F)C3)cc(C3CC3)n2)c(=O)n(C2CC2)c1] |
| 325 | 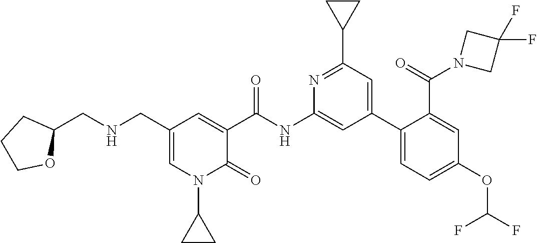 [SMILES: O=C(Nc1cc(-c2ccc(OC(F)F)cc2C(=O)N2CC(F)(F)C2)cc(C2CC2)n1)c1cc(CNC[C@@H]2CCCO2)cn(C2CC2)c1=O] |
| 326 | 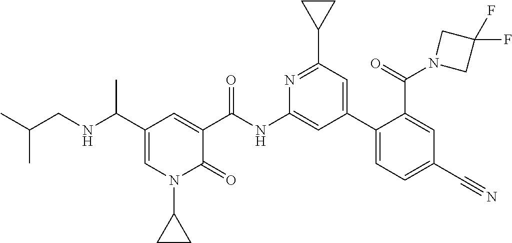 [SMILES: CC(C)CNC(C)c1cc(C(=O)Nc2cc(-c3ccc(C#N)cc3C(=O)N3CC(F)(F)C3)cc(C3CC3)n2)c(=O)n(C2CC2)c1] |
| 327 | 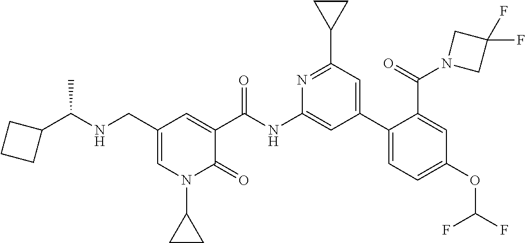 [SMILES: C[C@H](NCc1cc(C(=O)Nc2cc(-c3ccc(OC(F)F)cc3C(=O)N3CC(F)(F)C3)cc(C3CC3)n2)c(=O)n(C2CC2)c1)C1CCC1] |
| 328 | 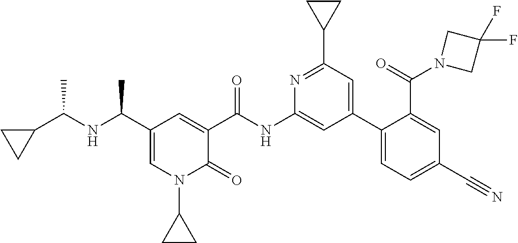 [SMILES: C[C@H](N[C@@H](C)C1CC1)c1cc(C(=O)Nc2cc(-c3ccc(C#N)cc3C(=O)N3CC(F)(F)C3)cc(C3CC3)n2)c(=O)n(C2CC2)c1] |
| 329 | 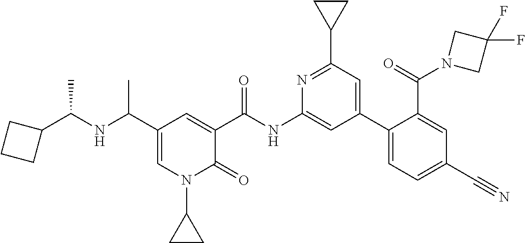 [SMILES: CC(N[C@@H](C)C1CCC1)c1cc(C(=O)Nc2cc(-c3ccc(C#N)cc3C(=O)N3CC(F)(F)C3)cc(C3CC3)n2)c(=O)n(C2CC2)c1] |
| 330 | 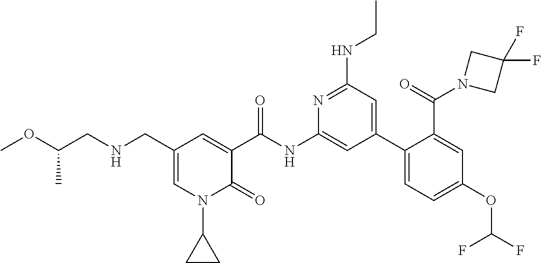 [SMILES: CCNc1cc(-c2ccc(OC(F)F)cc2C(=O)N2CC(F)(F)C2)cc(NC(=O)c2cc(CNC[C@H](C)OC)cn(C3CC3)c2=O)n1] |
| 331 | 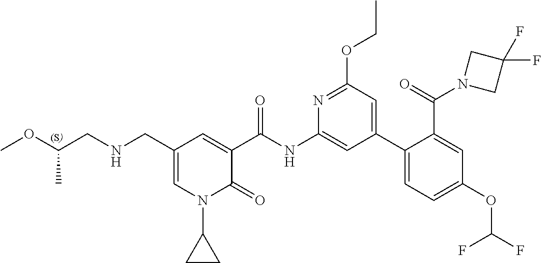 |
| 332 | 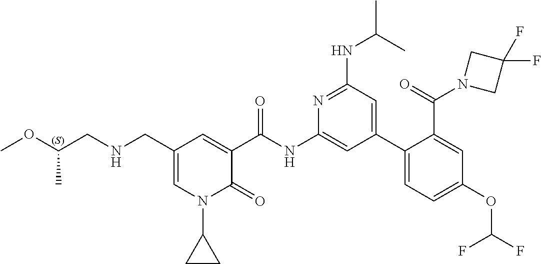 |
| 333 | 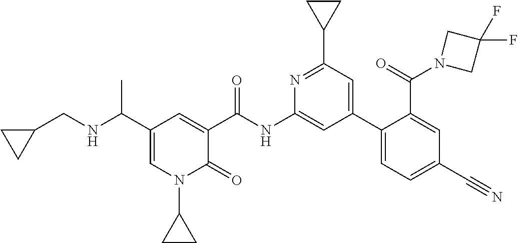 [SMILES: CC(NCC1CC1)c1cc(C(=O)Nc2cc(-c3ccc(C#N)cc3C(=O)N3CC(F)(F)C3)cc(C3CC3)n2)c(=O)n(C2CC2)c1] |
| 334 | 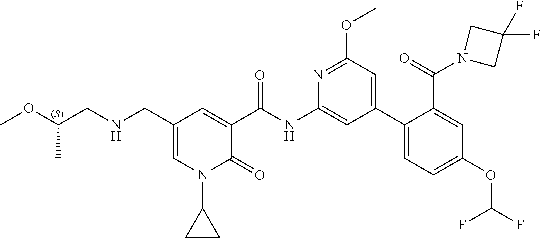 |
| 335 | 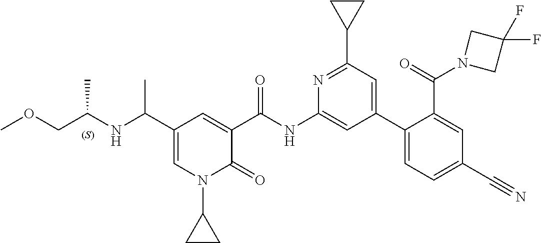 |
| 336 | 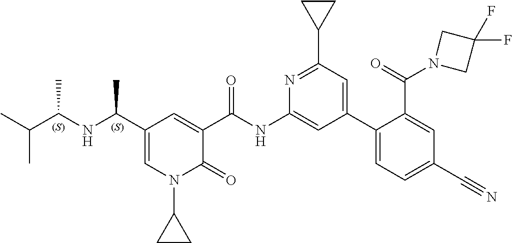 |
| 337 | 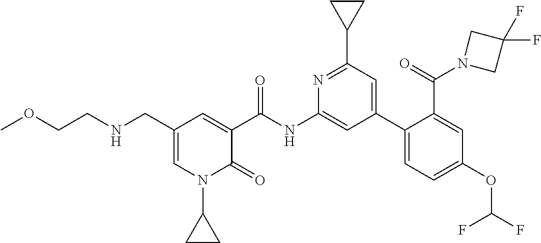 [SMILES: COCCNCc1cc(C(=O)Nc2cc(-c3ccc(OC(F)F)cc3C(=O)N3CC(F)(F)C3)cc(C3CC3)n2)c(=O)n(C2CC2)c1] |
| 338 | 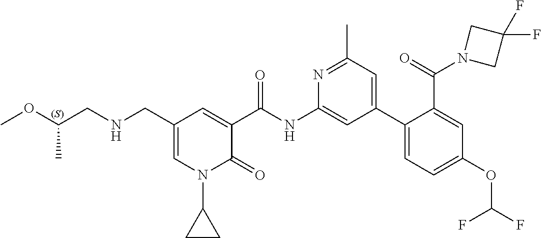 |
| 339 | 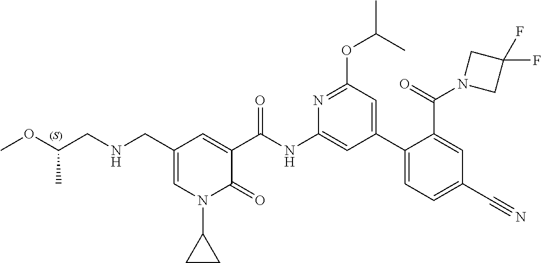 |
| 340 | 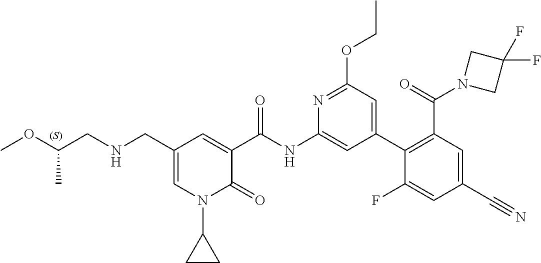 |
| 341 | 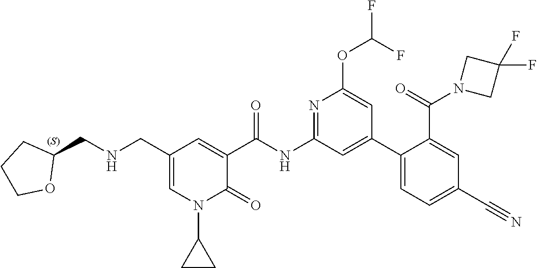 |
| 342 | 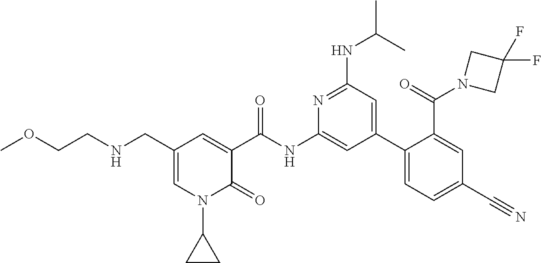 [SMILES: COCCNCc1cc(C(=O)Nc2cc(-c3ccc(C#N)cc3C(=O)N3CC(F)(F)C3)cc(NC(C)C)n2)c(=O)n(C2CC2)c1] |
| 343 | 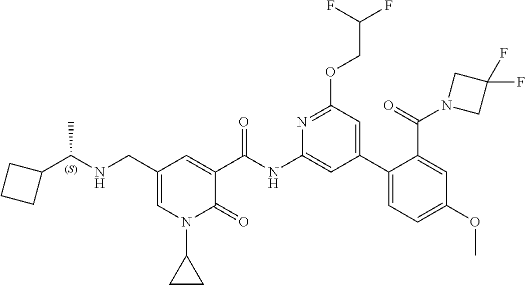 |
| 344 | 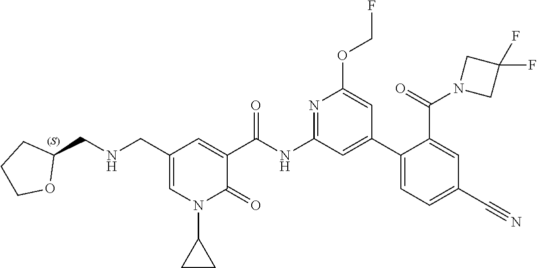 |
| 345 | 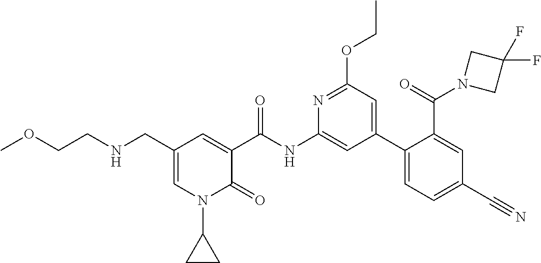 [SMILES: CCOc1cc(-c2ccc(C#N)cc2C(=O)N2CC(F)(F)C2)cc(NC(=O)c2cc(CNCCOC)cn(C3CC3)c2=O)n1] |
| 346 | 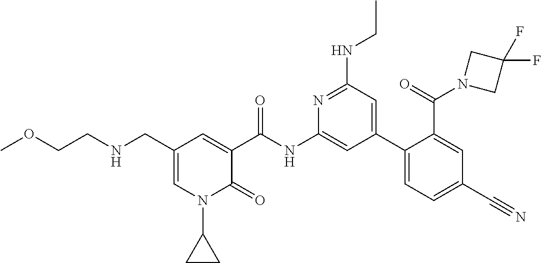 [SMILES: CCNc1cc(-c2ccc(C#N)cc2C(=O)N2CC(F)(F)C2)cc(NC(=O)c2cc(CNCCOC)cn(C3CC3)c2=O)n1] |
| 347 | 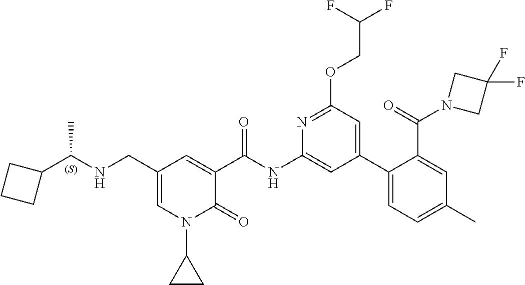 |
| 348 | 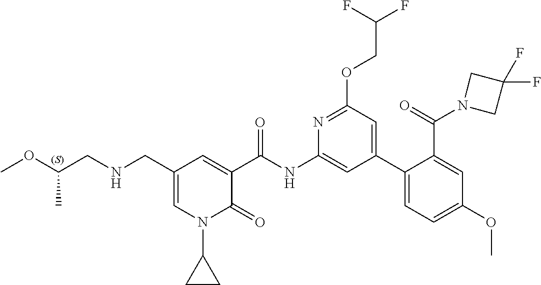 |
| 349 | 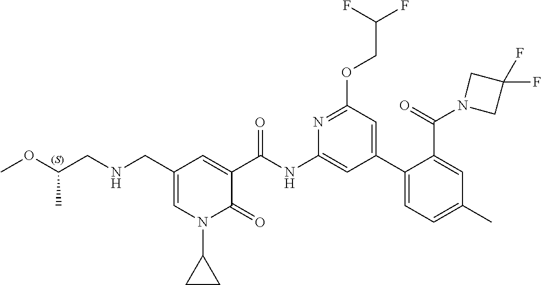 |
| 350 | 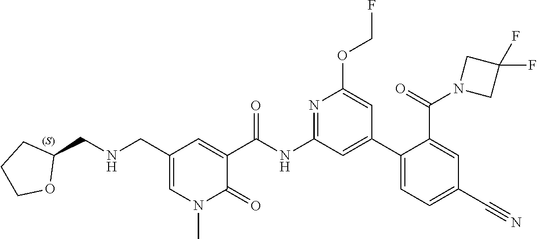 |
| 351 | 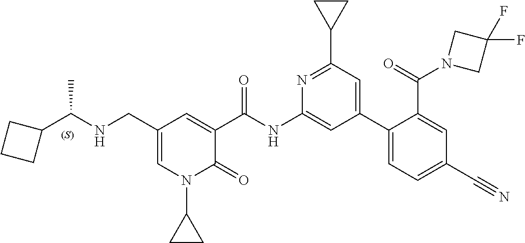 |
| 352 | 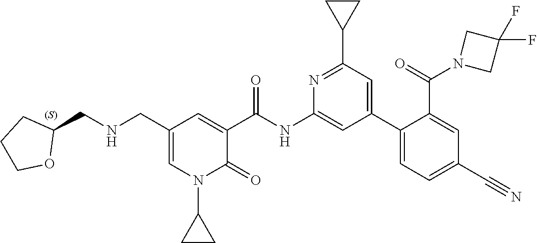 |
| 353 | 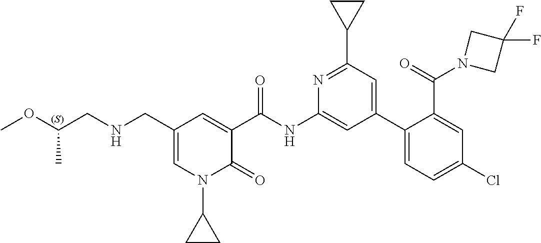 |
| 354 | 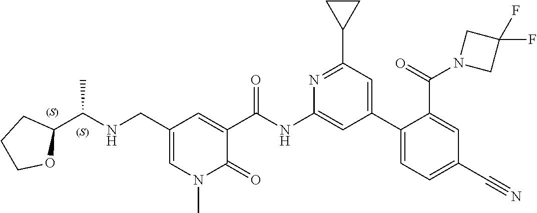 |
| 355 | 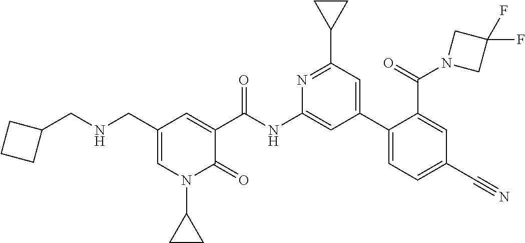 [SMILES: N#Cc1ccc(-c2cc(NC(=O)c3cc(CNCC4CCC4)cn(C4CC4)c3=O)nc(C3CC3)c2)c(C(=O)N2CC(F)(F)C2)c1] |
| 356 | 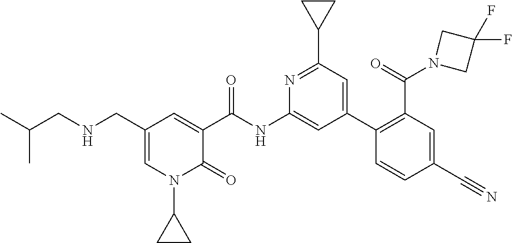 [SMILES: CC(C)CNCc1cc(C(=O)Nc2cc(-c3ccc(C#N)cc3C(=O)N3CC(F)(F)C3)cc(C3CC3)n2)c(=O)n(C2CC2)c1] |
| 357 | 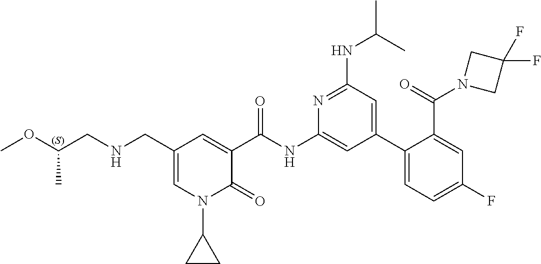 |
| 358 | 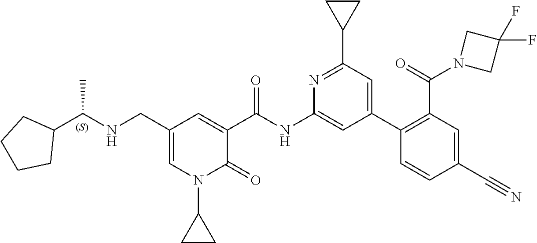 |
| 359 | 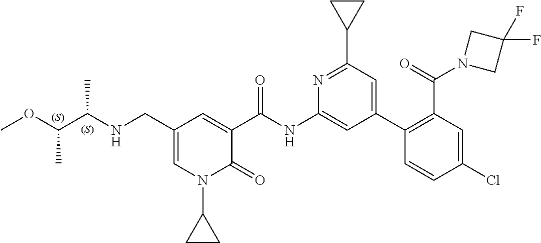 |
| 360 | 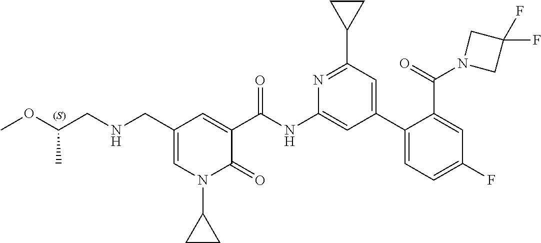 |
| 361 | 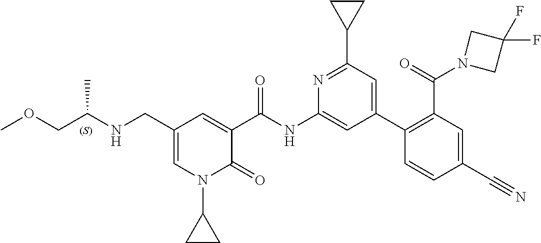 |
| 362 | 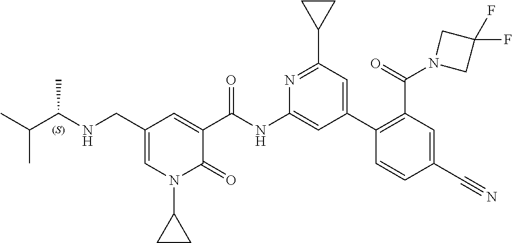 |
| 363 | 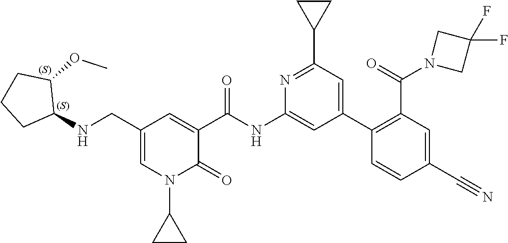 |
| 364 | 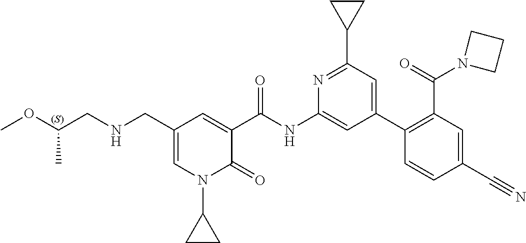 |
| 365 | 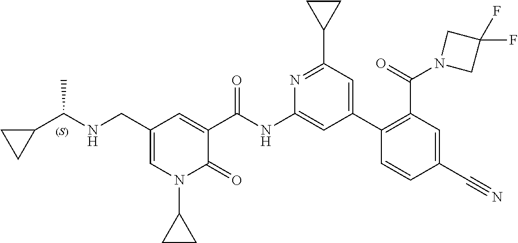 |
| 366 | 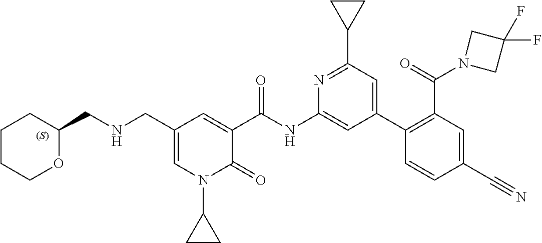 |
| 367 | 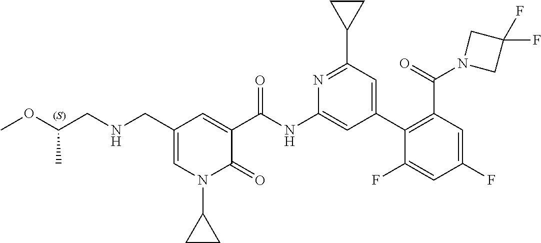 |
| 368 | 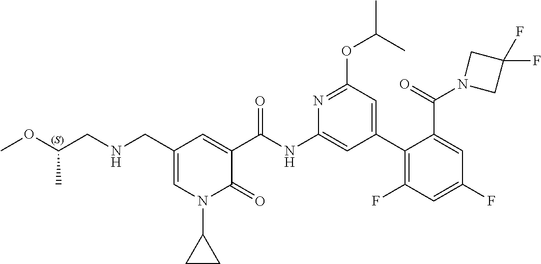 |
| 369 | 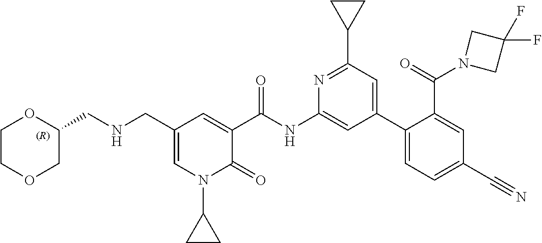 |
| 370 | 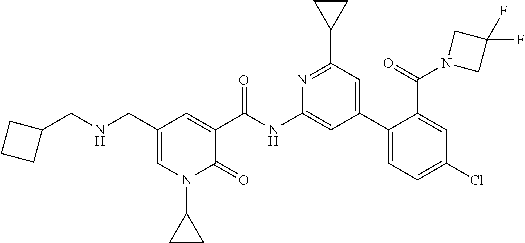 [SMILES: O=C(Nc1cc(-c2ccc(Cl)cc2C(=O)N2CC(F)(F)C2)cc(C2CC2)n1)c1cc(CNCC2CCC2)cn(C2CC2)c1=O] |
| 371 | 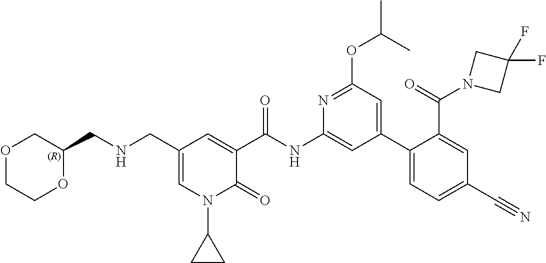 |
| 372 | 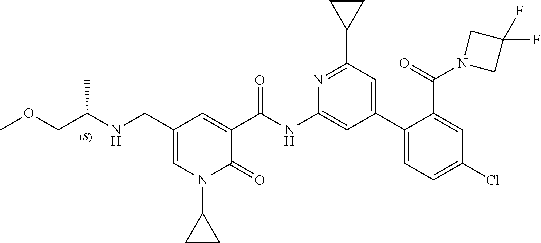 |
| 373 | 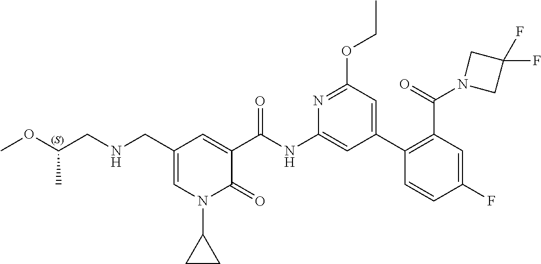 |
| 374 | 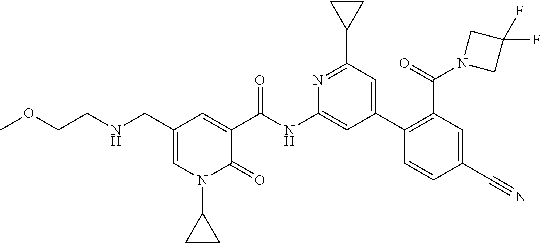 [SMILES: COCCNCc1cc(C(=O)Nc2cc(-c3ccc(C#N)cc3C(=O)N3CC(F)(F)C3)cc(C3CC3)n2)c(=O)n(C2CC2)c1] |
| 375 | 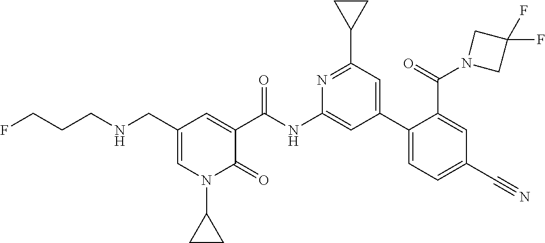 [SMILES: N#Cc1ccc(-c2cc(NC(=O)c3cc(CNCCCF)cn(C4CC4)c3=O)nc(C3CC3)c2)c(C(=O)N2CC(F)(F)C2)c1] |
| 376 | 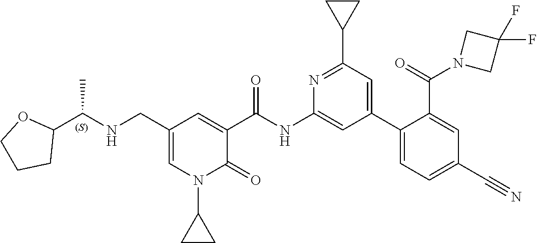 |
| 377 | 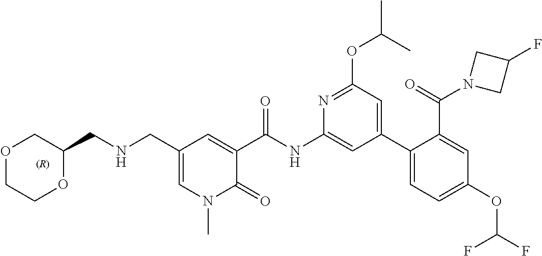 |
| 378 | 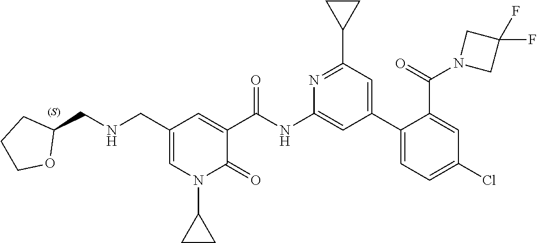 |
| 379 | 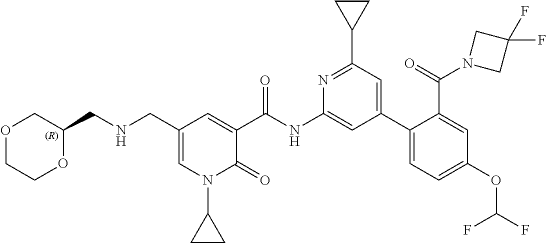 |
| 380 | 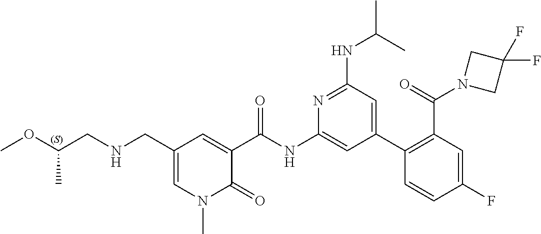 |
| 381 | 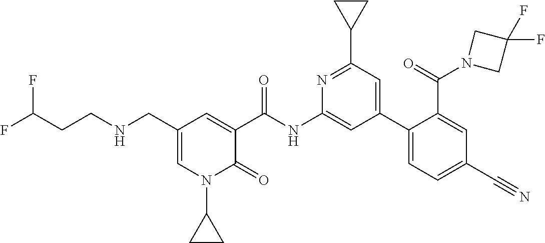 [SMILES: N#Cc1ccc(-c2cc(NC(=O)c3cc(CNCCC(F)F)cn(C4CC4)c3=O)nc(C3CC3)c2)c(C(=O)N2CC(F)(F)C2)c1] |
| 382 | 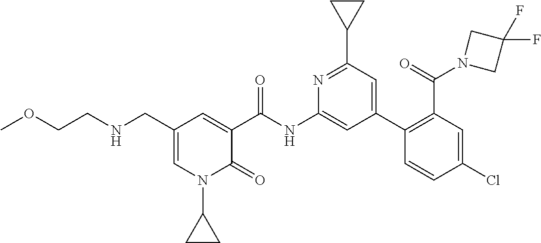 [SMILES: COCCNCc1cc(C(=O)Nc2cc(-c3ccc(Cl)cc3C(=O)N3CC(F)(F)C3)cc(C3CC3)n2)c(=O)n(C2CC2)c1] |
| 383 | 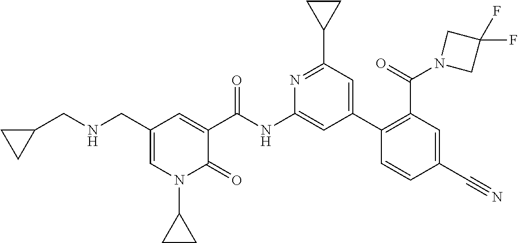 [SMILES: N#Cc1ccc(-c2cc(NC(=O)c3cc(CNCC4CC4)cn(C4CC4)c3=O)nc(C3CC3)c2)c(C(=O)N2CC(F)(F)C2)c1] |
| 384 | 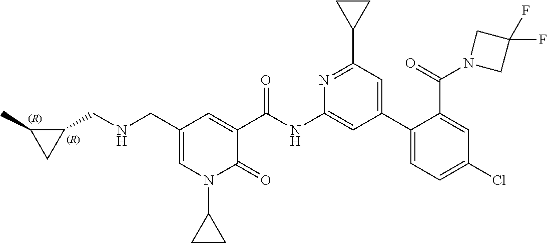 |
| 385 | 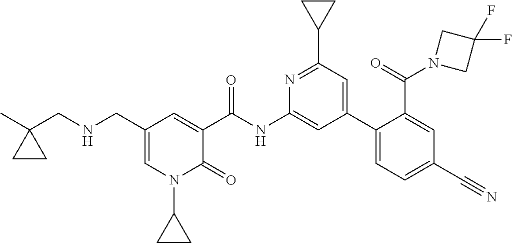 [SMILES: CC1(CNCc2cc(C(=O)Nc3cc(-c4ccc(C#N)cc4C(=O)N4CC(F)(F)C4)cc(C4CC4)n3)c(=O)n(C3CC3)c2)CC1] |
| 386 | 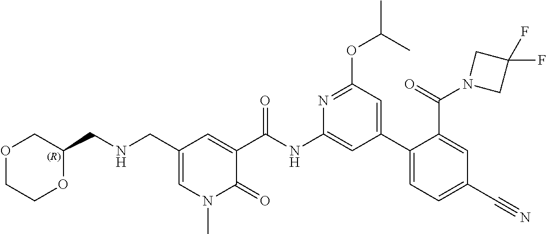 |
| 387 | 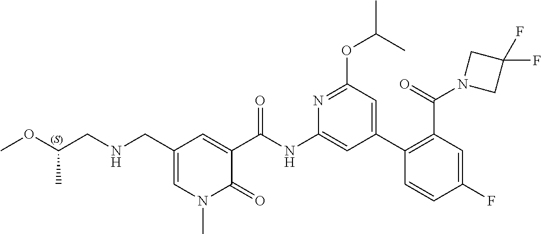 |
| 388 | 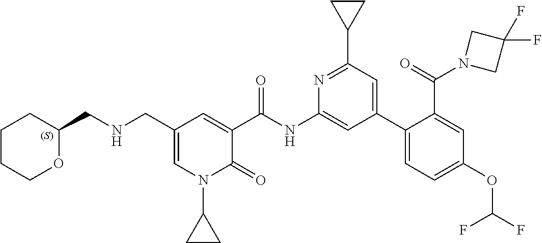 |
| 389 | 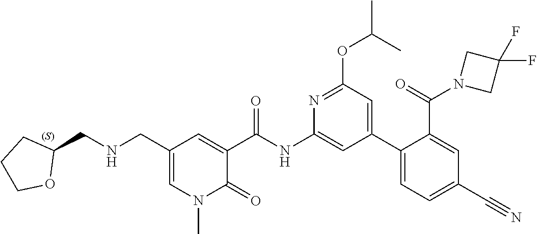 |
| 390 | 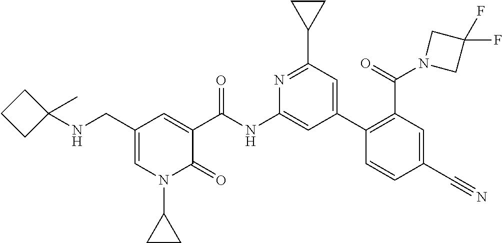 [SMILES: CC1(NCc2cc(C(=O)Nc3cc(-c4ccc(C#N)cc4C(=O)N4CC(F)(F)C4)cc(C4CC4)n3)c(=O)n(C3CC3)c2)CCC1] |
| 391 | 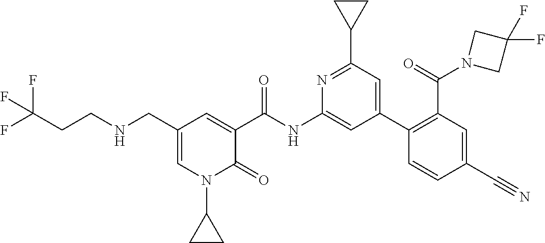 [SMILES: N#Cc1ccc(-c2cc(NC(=O)c3cc(CNCCC(F)(F)F)cn(C4CC4)c3=O)nc(C3CC3)c2)c(C(=O)N2CC(F)(F)C2)c1] |
| 392 | 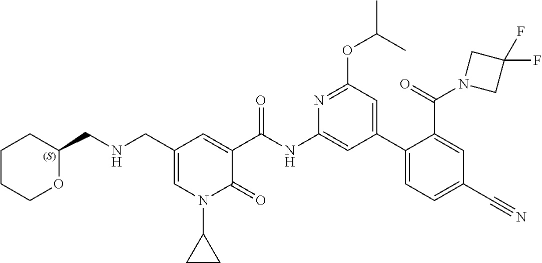 |
| 393 | 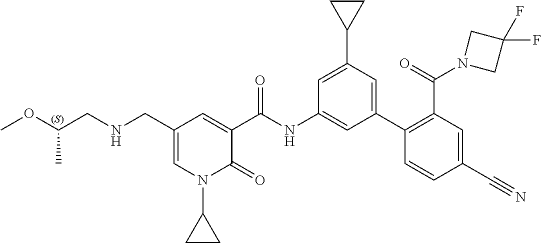 |
| 394 | 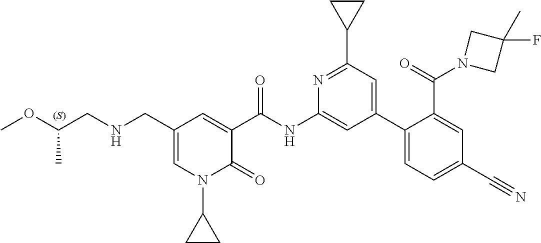 |
| 395 | 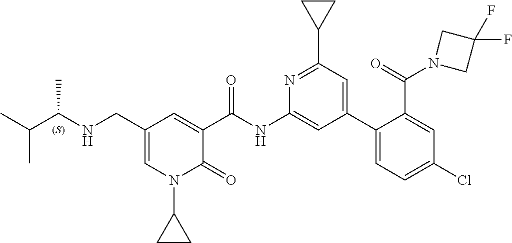 |
| 396 | 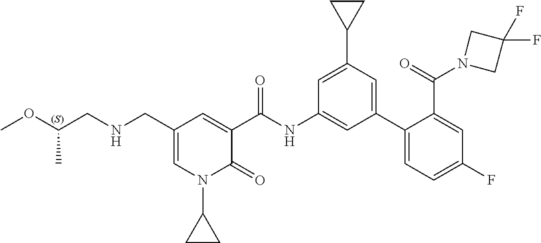 |
| 397 | 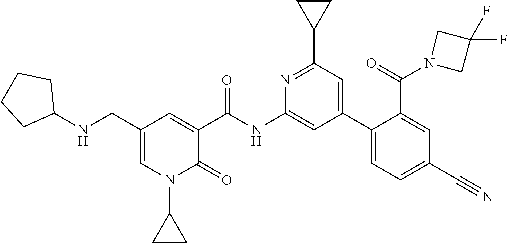 [SMILES: N#Cc1ccc(-c2cc(NC(=O)c3cc(CNC4CCCC4)cn(C4CC4)c3=O)nc(C3CC3)c2)c(C(=O)N2CC(F)(F)C2)c1] |
| 398 | 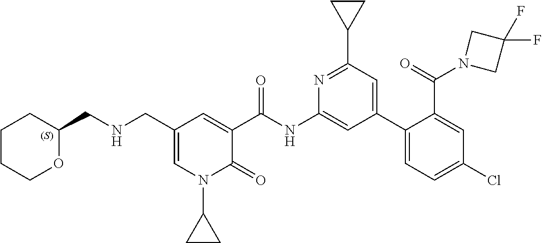 |
| 399 | 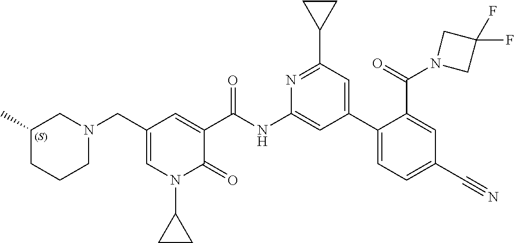 |
| 400 | 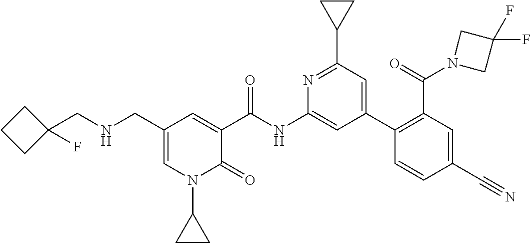 [SMILES: N#Cc1ccc(-c2cc(NC(=O)c3cc(CNCC4(F)CCC4)cn(C4CC4)c3=O)nc(C3CC3)c2)c(C(=O)N2CC(F)(F)C2)c1] |
| 401 | 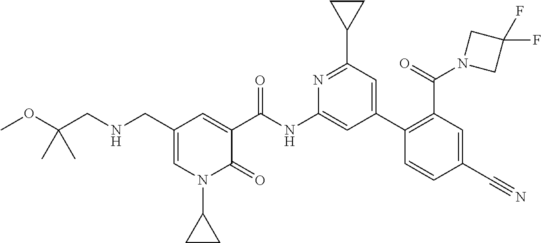 [SMILES: COC(C)(C)CNCc1cc(C(=O)Nc2cc(-c3ccc(C#N)cc3C(=O)N3CC(F)(F)C3)cc(C3CC3)n2)c(=O)n(C2CC2)c1] |
| 402 | 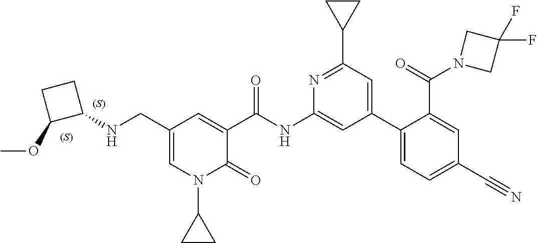 |
| 403 | 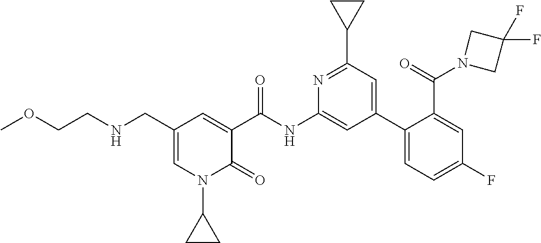 [SMILES: COCCNCc1cc(C(=O)Nc2cc(-c3ccc(F)cc3C(=O)N3CC(F)(F)C3)cc(C3CC3)n2)c(=O)n(C2CC2)c1] |
| 404 | 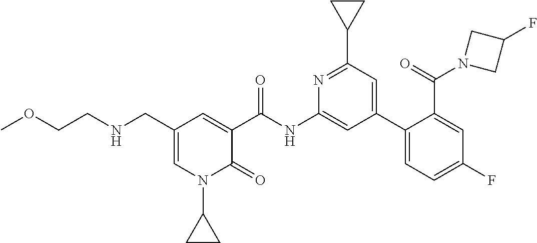 [SMILES: COCCNCc1cc(C(=O)Nc2cc(-c3ccc(F)cc3C(=O)N3CC(F)C3)cc(C3CC3)n2)c(=O)n(C2CC2)c1] |
| 405 | 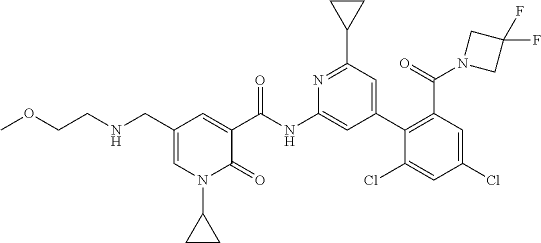 [SMILES: COCCNCc1cc(C(=O)Nc2cc(-c3c(Cl)cc(Cl)cc3C(=O)N3CC(F)(F)C3)cc(C3CC3)n2)c(=O)n(C2CC2)c1] |
| 406 | 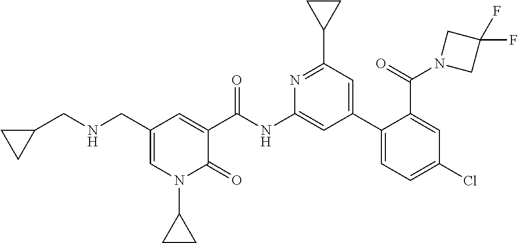 [SMILES: O=C(Nc1cc(-c2ccc(Cl)cc2C(=O)N2CC(F)(F)C2)cc(C2CC2)n1)c1cc(CNCC2CC2)cn(C2CC2)c1=O] |
| 407 | 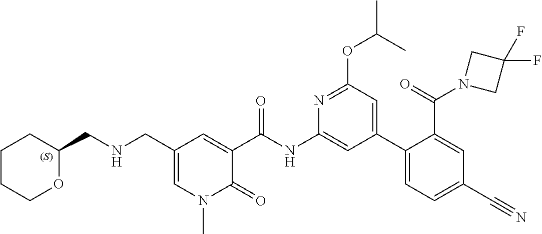 |
| 408 | 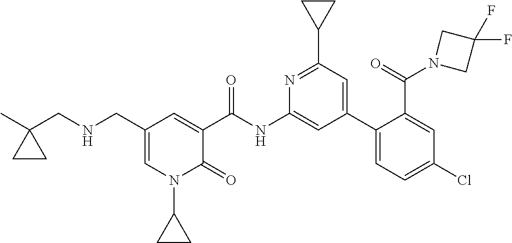 [SMILES: CC1(CNCc2cc(C(=O)Nc3cc(-c4ccc(Cl)cc4C(=O)N4CC(F)(F)C4)cc(C4CC4)n3)c(=O)n(C3CC3)c2)CC1] |
| 409 | 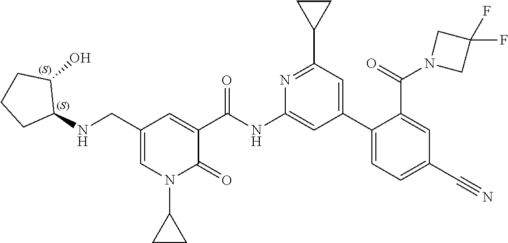 |
| 410 | 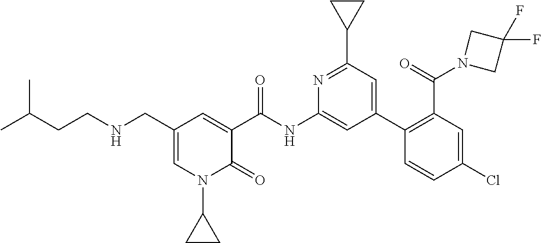 [SMILES: CC(C)CCNCc1cc(C(=O)Nc2cc(-c3ccc(Cl)cc3C(=O)N3CC(F)(F)C3)cc(C3CC3)n2)c(=O)n(C2CC2)c1] |
| 411 | 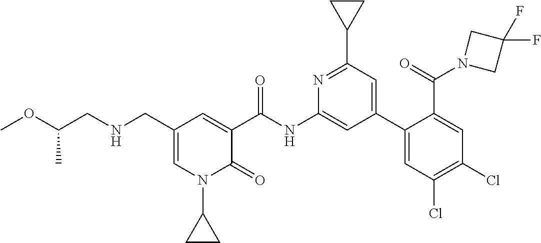 [SMILES: CO[C@@H](C)CNCc1cc(C(=O)Nc2cc(-c3cc(Cl)c(Cl)cc3C(=O)N3CC(F)(F)C3)cc(C3CC3)n2)c(=O)n(C2CC2)c1] |
| 412 | 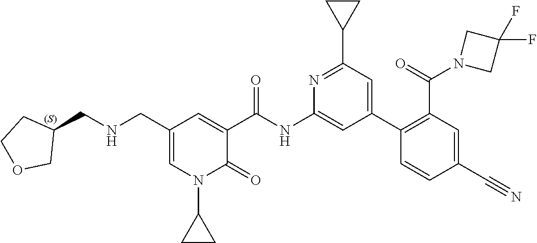 |

In some embodiments, the compound according to this disclosure is a pharmaceutically acceptable salt of a compound selected from the compounds provided in Table 1, Table 2, Table 3, Table 4, or Table 5.

### Therapeutic and Prophylactic Uses

The present disclosure provides methods for using compounds described herein in the preparation of a medicament. In some embodiments, the medicament is for inhibiting Cbl-b. As used herein, the terms “inhibit”, “inhibition” and the like refer to the ability of a compound to decrease the function or activity of a particular target, e.g., Cbl-b. The decrease is preferably at least 50% and may be, for example, at least about 55%, at least about 60%, at least about 65%, at least about 70%, at least about 75%, at least about 80%, at least about 85%, at least about 90%, or at least about 95%. The present disclosure also encompasses the use of the compounds described herein in the preparation of a medicament for the treatment or prevention of diseases, disorders, and/or conditions that would benefit from inhibition of Cbl-b. As one example, the present disclosure encompasses the use of the compounds described herein in the preparation of a medicament for the treatment of cancer. In another example, the present disclosure encompasses the use of the compounds described herein in the preparation of a medicament for the treatment of an infectious disease, optionally a viral infection. In some embodiments of the aforementioned methods, the compounds described herein are used in combination with at least one additional therapy, examples of which are set forth elsewhere herein.

Cbl-b is an E3 ubiquitin ligase that acts by ubiquitinating proteins leading to their degradation or altered subcellular localization. More specifically, Cbl-b acts by binding ubiquitin-conjugating enzyme (E2) loaded with ubiquitin and substrate to facilitate formation of an isopeptide bond between the C-terminal carboxyl of ubiquitin and the F-amino group of a substrate lysine side chain or free N-terminal amino group. Through this activity, Cbl-b functions, in one aspect, as a negative regulator of immune cell activation. For example, Cbl-b inhibits T cell activation through ubiquitination of intracellular signaling proteins, including but not limited to pTYR-containing proteins (e.g., ZAP-70, etc.), p85 regulatory subunit of phosphatidynlinositol 3 kinase (PI3K), PLCγ1, and PKCθ. Cbl-b is also believed to negatively regulate cytokine-induced or target-induced NK cell cytotoxicity and cytokine production. Cbl-b has also been implicated in immunosuppressive signaling pathways, such as PD-1, CTLA-4, CD155, and TGF-β.

As demonstrated herein, the use of compounds described herein potently inhibits Cbl-b activity, resulting in increased immune cell activity. Diseases, disorders, and/or conditions that would benefit from Cbl-b inhibition may include those where greater immune cell (e.g., T cell, NK cell, etc.) activation is desired and/or there is limited immune cell stimulation, for example, due to low antigen density, poor quality neoantigen, high PD-L1 expression, or combinations thereof.

Accordingly, in some embodiments, the compounds described herein are administered to a subject in need thereof in an amount effective to inhibit Cbl-b activity. In one example, a measure of Cbl-b inhibition may be decreased ubiquitination of intracellular signaling proteins targeted by Cbl-b. Non-limiting examples of intracellular signaling proteins targeted by Cbl-b include pTYR-containing proteins (e.g., ZAP-70, etc.), p85 regulatory subunit of phosphatidynlinositol 3 kinase (PI3K), PLCγ1, and PKCθ. Cbl-b activity may be assessed using primary immune cells (e.g., T cells, NK cells) obtained from a peripheral blood sample or a tissue sample (e.g., a tumor sample) that was obtained from the subject. Activity may be determined, for example, by comparison to a previous sample obtained from the subject (i.e., prior to administration of the compound) or by comparison to a reference value for a control group (e.g., standard of care, a placebo, etc.).

Alternatively or in addition, in some embodiments, the compounds described herein are administered to a subject in need thereof in an amount effective to increase immune cell expansion, proliferation, activation and/or activity, as compared to a suitable control (e.g., a subject receiving standard of care, a subject receiving no treatment or a placebo treatment, etc.). Immune cell expansion, proliferation, activation and activity may be assessed using cells obtained from a peripheral blood sample or a tissue sample (e.g., a tumor sample) that was obtained from the subject. Immune cell numbers in tissue or blood may be quantified (absolute numbers or relative numbers) by immunophenotyping, i.e., a process of using antibodies (or other antigen-specific reagent) to detect and quantify cell-associated antigens. Lymphoid cell markers may include but are not limited to CD3, CD4, CD8, CD16, CD25, CD39, CD45, CD56, CD103, CD127, and FOXP3. CD4 and CD8 can distinguish T cell with different effector functions (e.g., CD4+ T cells and CD8+ T cells). Co-expression of different cell markers can further distinguish sub-groups. For example, co-expression of CD39 and CD103 can differentiate tumor-specific T cells (CD8+CD39+CD103+ T cells) from bystander T cells in the tumor microenvironment (TME). For myeloid cells, suitable markers may include but are not limited to CD14, CD68, CD80, CD83, CD86, CD163, and CD206. Ki67 is a non-limiting example of a suitable marker of cell proliferation, such that an increase in Ki67 positive cells (e.g., CD8+ T cells, NK cells, etc.) as compared to a reference sample indicate cell proliferation. The term “activation” refers to the state of an immune cell that has been sufficiently primed to induce detectable effector functions (i.e., immune cell activity) upon stimulation. For example, T cells may be stimulated through the TCR/CD3 complex alone or with one or more secondary costimulatory signals. Non-limiting examples of measures of increased immune cell activity (i.e. effector function) may include increased expression, production and/or secretion of chemokines, pro-inflammatory cytokines and/or cytotoxic factors, increased cytotoxic activity, and increased gene expression and/or cell surface markers related to immune cell function and immune signaling. Examples of pro-inflammatory cytokines include, but are not limited to, IL-1a, IL-1b, IL-2, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of cytotoxic factors include, but are not limited to, granzyme A, granzyme B, soluble Fas ligand (sFasL), and perforin.

In some embodiments, the compounds described herein are administered to a subject in need thereof in an amount effective to increase T cell expansion, proliferation, activity, or any combination thereof. In certain embodiments, the T cells are CD8+ T cells, optionally tumor infiltrating CD8+ T cells and/or antigen experienced CD8+ T cells. In some embodiments, the T cells are CD8+CD39+CD103+ T cells. In embodiments directed to increased T cell activation and/or activity, measures of increased T cell activity may be increased T cell expression, production or secretion of chemokines, pro-inflammatory cytokines (e.g., IFNγ, TNF-α, IL-2, etc.) and/or cytotoxic factors (e.g. perforin, Granzyme B, etc.); increased pro-inflammatory cytokine levels in the tumor microenvironment; increased T cell receptor (TCR) signaling; increased glucose uptake; increased glycolysis; and increased killing of cancer cells. In some embodiments, the compounds described herein are administered to a subject in need thereof in an amount effective to increase activity, optionally wherein a measure of T cell activity is production and/or secretion of one or more pro-inflammatory cytokine, optionally wherein one or more pro-inflammatory cytokine is IFNγ, TNF-α, or IL-2.

In some embodiments, the compounds described herein are administered to a subject in need thereof in an amount effective to increase NK cell expansion, proliferation, activity, or any combination thereof. In embodiments directed to increased NK cell activity, measures of increased NK cell activity may be increased NK cell expression, production or secretion of chemokines, inflammatory cytokines (e.g., IFNγ, TNF-α, IL-2, etc.) and/or cytotoxic factors (e.g. perforin, Granzyme B, etc.); increased inflammatory cytokine levels in the tumor microenvironment; and increased killing of cancer cells.

Alternatively or in addition, in some embodiments, the compounds described herein are administered to a subject in need thereof to treat and/or prevent cancer or a cancer-related disease, disorder or condition. In some embodiments, the compounds described herein are administered to a subject in need thereof to treat cancer, optionally in combination with at least one additional therapy, examples of which are set forth elsewhere herein.

Alternatively or in addition, in some embodiments, the compounds described herein are administered to a subject in need thereof to treat and/or prevent an infection. In some embodiments, the compounds described herein are administered to a subject in need thereof to treat and/or prevent a viral infection. In some embodiments, the viral infection is a disease caused by hepatitis C virus (HCV), human papilloma virus (HPV), cytomegalovirus (CMV), herpes simplex virus (HSV), Epstein-Barr virus (EBV), varicella zoster virus, coxsackie virus, human immunodeficiency virus (HIV), or lymphocytic choriomeningitis virus (LCMV).

Alternatively or in addition, in some embodiments, the compounds described herein are brought into contact with an immune cell or a plurality of immune cells, in vitro or ex vivo, in an amount effective to increase proliferation, activation or activity of the immune cell(s). In some embodiments, the immune cell(s) may be allogenic immune cell(s) collected from one or more subjects. In some embodiments, the immune cell(s) may be autologous immune cell(s) collected from a subject in need of treatment. In certain embodiments, the cells may be “(re)programmed” allogenic immune cells produced from immune precursor cells (e.g., lymphoid progenitor cells, myeloid progenitor cells, common dendritic cell precursor cells, stem cells, induced pluripotent stem cells, etc.). In various embodiments, the immune cells may be genetically modified to target the cells to a specific antigen and/or enhance the cells' anti-tumor effects (e.g., engineered T cell receptor (TCR) cellular therapies, chimeric antigen receptor (CAR) cellular therapies, etc.). In some embodiments, the immune cell(s) are then administered to a subject in need thereof to treat and/or prevent cancer or a cancer-related disease, disorder or condition. In some embodiments, the immune cells are administered to a subject in need thereof to treat cancer, optionally in combination with at least one additional therapy, examples of which are set forth elsewhere herein.

In one or more embodiments, the compounds described herein are useful in the treatment and/or prophylaxis of cancer (e.g., carcinomas, sarcomas, leukemias, lymphomas, myelomas, etc.). In certain embodiments, the cancer may be locally advanced and/or unresectable, metastatic, or at risk of becoming metastatic. Alternatively, or in addition, the cancer may be recurrent or no longer responding to a treatment, such as a standard of care treatment known to one of skill in the art. In some embodiments, the cancer is resistant to treatment with immune checkpoint inhibitors (e.g., anti-PD-1 therapy), and/or chemotherapy (e.g., platinum-based chemotherapy). Exemplary types of cancer contemplated by this disclosure include cancer of the genitourinary tract (e.g., gynecologic, bladder, kidney, renal cell, penile, prostate, testicular, etc.), breast, gastrointestinal tract (e.g., esophagus, oropharynx, stomach, small or large intestines, colon, or rectum), bone, bone marrow, skin (e.g., melanoma), head and neck, liver, gall bladder, bile ducts, heart, lung, pancreas, salivary gland, adrenal gland, thyroid, brain (e.g., gliomas), ganglia, central nervous system (CNS), peripheral nervous system (PNS), the hematopoietic system (i.e., hematological malignancies), the immune system (e.g., spleen or thymus), and cancers associated with Von Hippel-Lindau disease (VHL).

In some embodiments, the compounds according to this disclosure are useful in the treatment and/or prophylaxis of hematological malignancies. Exemplary types of cancer affecting the hematopoietic system include leukemias, lymphomas and myelomas, including acute myeloid leukemia, adult T-cell leukemia, T-cell large granular lymphocyte leukemia, acute lymphoblastic leukemia, chronic lymphocytic leukemia, chronic myelogenous leukemia, acute monocytic leukemia, Hodgkin's and Non-Hodgkin's lymphoma, Diffuse large B Cell lymphoma, and multiple myeloma. In a specific embodiment, the compounds according to this disclosure are useful in the treatment of Diffuse large B Cell lymphoma, optionally Diffuse large B Cell lymphoma with Richter transformation.

In another embodiment, the compounds according to this disclosure are useful in the treatment and/or prophylaxis of solid tumors. The solid tumor may be, for example, ovarian cancer, endometrial cancer, breast cancer, lung cancer (small cell or non-small cell), colon cancer, prostate cancer, cervical cancer, biliary cancer, pancreatic cancer, gastric cancer, esophageal cancer, liver cancer (hepatocellular carcinoma), kidney cancer (renal cell carcinoma), head-and-neck tumors, mesothelioma, melanoma, sarcomas, central nervous system (CNS) hemangioblastomas, and brain tumors (e.g., gliomas, such as astrocytoma, oligodendroglioma and glioblastomas).

In another embodiment, the compounds according to this disclosure are useful in the treatment and/or prophylaxis of breast cancer, genitourinary cancer, gastrointestinal cancer, lung cancer, skin cancer, or a combination thereof.

In some embodiments, the compounds according to this disclosure are useful in the treatment of breast cancer. In further embodiments, the breast cancer is hormone receptor positive (e.g., Erα-positive breast cancer, PR-positive breast cancer, Erα-positive and PR-positive breast cancer), HER2 positive breast cancer, HER2 over-expressing breast cancer, or any combination thereof. In still further embodiments, the breast cancer is triple negative breast cancer (TNBC).

In some embodiments, the compounds according to this disclosure are useful in the treatment of genitourinary cancer. In further embodiments, the genitourinary cancer is gynecologic cancer. In still further embodiments, the gynecologic cancer is cervical cancer, ovarian cancer (e.g., epithelial ovarian cancer (EOC)), vaginal cancer, vulvar cancer, endometrial cancer, peritoneal cancer, or fallopian tube carcinoma. In still further embodiments, the genitourinary cancer is urothelial cancer. In still further embodiments, the genitourinary cancer is prostate cancer, optionally castration-resistant prostate cancer. In further embodiments, the genitourinary cancer is bladder cancer. In still further embodiments, the genitourinary cancer is peritoneal cancer, optionally primary peritoneal cancer.

In some embodiments, the compounds according to this disclosure are useful in the treatment of head and neck cancer. In further embodiments, the head and neck cancer is head and neck squamous cell carcinoma (HNSCC).

In some embodiments, the compounds according to this disclosure are useful in the treatment of skin cancer. In further embodiments, the skin cancer is melanoma.

In some embodiments, the compounds according to this disclosure are useful in the treatment of lung cancer. In further embodiments, the lung cancer is mesothelioma or non-small cell lung cancer (NSCLC). In still further embodiments, the NSCLC is lung squamous cell carcinoma or lung adenocarcinoma. In further embodiments, the mesothelioma is malignant pleural mesothelioma (MPM).

In some embodiments, the compounds according to this disclosure are useful in the treatment of gastrointestinal (GI) cancer. In some embodiments, the gastrointestinal cancer is upper GI cancer, such as esophageal or gastric cancer. In further embodiments, the upper GI cancer is an adenocarcinoma, a squamous cell carcinoma, or any combination thereof. In still further embodiments, the upper GI cancer is esophageal adenocarcinoma (EAC), esophageal squamous cell carcinoma (ESCC), gastroesophageal junction adenocarcinoma (GEJ), gastric adenocarcinoma (also referred to herein as “gastric cancer”) or any combination thereof. In some embodiments, the gastrointestinal cancer is lower GI cancer. In further embodiments, the lower GI cancer is colorectal cancer.

In some embodiments, the compounds according to this disclosure are useful in the treatment of a neuroendocrine tumor. In further embodiments, the neuroendocrine tumor is pancreatic neuroendocrine tumor, pheochromocytoma, paraganglioma, or a tumor of the adrenal gland.

In some embodiments, the compounds according to this disclosure are useful in the treatment of brain cancer. In further embodiments, the brain cancer is a glioma. In still further embodiments, the glioma is an astrocytoma, an oligodendroglioma, or a glioblastoma.

In some embodiments, the compounds according to this disclosure are useful in the treatment of kidney cancer. In further embodiments, the kidney cancer is renal cell carcinoma. In still further embodiments, the renal cell carcinoma is clear cell renal carcinoma.

In some embodiments, the compounds according to this disclosure are useful in the treatment of pancreatic cancer. In further embodiments, the pancreatic cancer is pancreatic neuroendocrine tumor or pancreatic adenocarcinoma.

In the aforementioned embodiments, the methods of the present disclosure may be practiced in an adjuvant setting or neoadjuvant setting, optionally in the treatment of locally advanced, unresectable, or metastatic cancer. Alternatively or in addition, the methods described herein may be indicated as a first line, second line, third line, or greater line of treatment, optionally in the treatment of locally advanced, unresectable, or metastatic cancer.

The present disclosure also provides methods of treating or preventing other cancer-related diseases, disorders or conditions. The use of the term(s) cancer-related diseases, disorders and conditions is meant to refer broadly to conditions that are associated, directly or indirectly, with cancer and non-cancerous proliferative disease, and includes, e.g., angiogenesis, precancerous conditions such as dysplasia, and non-cancerous proliferative diseases disorders or conditions, such as benign proliferative breast disease and papillomas. For clarity, the term(s) cancer-related disease, disorder and condition do not include cancer per se.

In general, the disclosed methods for treating or preventing cancer, or a cancer-related disease, disorder or condition, in a subject in need thereof comprise administering to the subject a compound disclosed herein, or a pharmaceutically acceptable salt thereof. In some embodiments, the present disclosure provides methods for treating or preventing cancer, or a cancer-related disease, disorder or condition with a compound disclosed herein, or a pharmaceutically acceptable salt thereof, and at least one additional therapy, examples of which are set forth elsewhere herein.

In particular embodiments of the present disclosure, the compounds are used to increase or enhance an immune response to an antigen by providing adjuvant activity. In a particular embodiment, at least one antigen or vaccine is administered to a subject in combination with at least one compound of the present disclosure to prolong an immune response to the antigen or vaccine. Therapeutic compositions are also provided which include at least one antigenic agent or vaccine component, including, but not limited to, viruses, bacteria, and fungi, or portions thereof, proteins, peptides, tumor-specific antigens, and nucleic acid vaccines, in combination with at least one compound of the present disclosure.

In some instances, the methods according to this disclosure may be provided in selected patients, for example subjects identified as having in a relevant tissue or sample, e.g., detectable PD-L1 expression, high microsatellite instability, high tumor mutational burden, or any combination thereof. In some instances, the subject is identified as having an oncogene driven cancer that has a mutation in at least one gene associated with the cancer.

In some embodiments, patients are selected by assessing the expression of relevant biomarkers, e.g., PD-L1 expression, microsatellite instability markers, etc., in a relevant sample, such as a peripheral blood sample or a tumor biopsy, using immunohistochemistry, immunophenotyping, PCR-based amplification, RNA sequencing, or other clinically validated assay. In one embodiment, the disclosure provides a method of treating cancer in a patient having (i) detectable PD-L1 expression, (ii) elevated PD-L1 expression, (iii) variability in the size of one, two, or more microsatellite repeats compared to normal cells, or (iv) any combination of (i) to (iii) by administering a compound as described herein. In another embodiment, the disclosure provides a method of treating cancer in a patient having (i) detectable PD-L1 expression, (ii) elevated PD-L1 expression, (iii) variability in the size of one, two, or more microsatellite repeats compared to normal cells, or (iv) any combination of (i) to (iii) by administering a therapeutically effective amount of a compound as described herein. In still another embodiment, the disclosure provides a method of administering a therapeutically effective amount of a compound as described herein to an individual for the treatment of cancer based on a determination of the relative amount of PD-L1 expression. In yet another embodiment, the disclosure provides a method of administering a therapeutically effective amount of a compound described herein to an individual for the treatment of cancer, the method comprising measuring PD-L1expression and/or microsatellite instability in a sample obtained from an individual, for example by immunohistochemistry, immunophenotyping, PCR-based amplification, or other clinically validated test, and administering a therapeutically effective amount of the compound to the individual whose sample contained detectable PD-L1 expression.

### Routes of Administration

In some embodiments, pharmaceutical compositions containing a compound according to this disclosure may be in a form suitable for oral administration. Oral administration may involve swallowing the formulation thereby allowing the compound to be absorbed into the bloodstream in the gastrointestinal tract. Alternatively, oral administration may involve buccal, lingual or sublingual administration, thereby allowing the compound to be absorbed into the blood stream through oral mucosa.

In another embodiment, the pharmaceutical compositions containing a compound according to this disclosure may be in a form suitable for parenteral administration. Forms of parenteral administration include, but are not limited to, intravenous, intraarterial, intramuscular, intradermal, intraperitoneal, intrathecal, intracisternal, intracerebral, intracerebroventricular, intraventricular, and subcutaneous. Pharmaceutical compositions suitable for parenteral administration may be formulated using suitable aqueous or non-aqueous carriers. Depot injections, which are generally administered subcutaneously or intramuscularly, may also be utilized to release the compounds disclosed herein over a defined period of time.

Other routes of administration are also contemplated by this disclosure, including, but not limited to, nasal, vaginal, intraocular, rectal, topical (e.g., transdermal), and inhalation.

Particular embodiments of the present disclosure contemplate oral administration or parenteral administration.

### Pharmaceutical Compositions

The compounds of the present disclosure may be in the form of compositions suitable for administration to a subject. In general, such compositions are pharmaceutical compositions comprising a compound according to this disclosure or a pharmaceutically acceptable salt thereof and one or more pharmaceutically acceptable excipients. In some embodiments, the pharmaceutical composition comprises a compound according to this disclosure and one or more pharmaceutically acceptable excipients. In certain embodiments, the compound may be present in an effective amount. The pharmaceutical compositions may be used in the methods of the present disclosure; thus, for example, the pharmaceutical compositions comprising a compound according to this disclosure can be administered to a subject in order to practice the therapeutic and prophylactic methods and uses described herein.

The pharmaceutical compositions of the present disclosure can be formulated to be compatible with the intended method or route of administration. Routes of administration may include those known in the art. Exemplary routes of administration are oral and parenteral. Furthermore, the pharmaceutical compositions may be used in combination with one or more other therapies described herein in order to treat or prevent the diseases, disorders and conditions as contemplated by the present disclosure. In one embodiment, one or more other therapeutic agents contemplated by this disclosure are included in the same pharmaceutical composition that comprises the compound according to this disclosure. In another embodiment, the one or more other therapeutical agents are in a composition that is separate from the pharmaceutical composition comprising the compound according to this disclosure.

In one aspect, the compounds described herein may be administered orally. Oral administration may be via, for example, capsule or tablets. In making the pharmaceutical compositions that include the compounds of the present disclosure (e.g., a compound of Formula I), or a pharmaceutically acceptable salt thereof, the tablet or capsule includes at least one pharmaceutically acceptable excipient. Non-limiting examples of pharmaceutically acceptable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, sterile water, syrup, and methyl cellulose. Additional pharmaceutically acceptable excipients include lubricating agents such as talc, magnesium stearate, and mineral oil; wetting agents; emulsifying and suspending agents; preserving agents such as methyl and propylhydroxy-benzoates.

In another aspect, the compounds of the present disclosure, or a pharmaceutically acceptable salt thereof, may be administered parenterally, for example by intravenous injection. A pharmaceutical composition appropriate for parenteral administration may be formulated in solution for injection or may be reconstituted for injection in an appropriate system such as a physiological solution. Such solutions may include sterile water for injection, salts, buffers, and tonicity excipients in amounts appropriate to achieve isotonicity with the appropriate physiology.

The pharmaceutical compositions described herein may be stored in an appropriate sterile container or containers. In some embodiments, the container is designed to maintain stability for the pharmaceutical composition over a given period of time.

### Administering

In general, the disclosed methods comprise administering a compound described herein, or a composition thereof, in an effective amount to a subject in need thereof. An “effective amount” with reference to a Cbl-b inhibitor of the present disclosure means an amount of the compound that is sufficient to engage the target (e.g., by inhibiting the target) at a level that is indicative of the potency of the compound. For Cbl-b, target engagement can be determined by one or more biochemical or cellular assays resulting in an EC50, ED50, EC90, IC50, or similar value which can be used as one assessment of the potency of the compound. Assays for determining target engagement include, but are not limited to, those described in the Examples. The effective amount may be administered as a single quantity or as multiple, smaller quantities (e.g., as one tablet with “x” amount, as two tablets each with “x/2” amount, etc.).

In some embodiments, the disclosed methods comprise administering a therapeutically effective amount of a compound described herein to a subject in need thereof. As used herein, the phrase “therapeutically effective amount” with reference to compound disclosed herein means a dose regimen (i.e., amount and interval) of the compound that provides the specific pharmacological effect for which the compound is administered to a subject in need of such treatment. For prophylactic use, a therapeutically effective amount may be effective to eliminate or reduce the risk, lessen the severity, or delay the onset of the disease, including biochemical, histological and/or behavioral signs or symptoms of the disease. For treatment, a therapeutically effective amount may be effective to reduce, ameliorate, or eliminate one or more signs or symptoms associated with a disease, delay disease progression, prolong survival, decrease the dose of other medication(s) required to treat the disease, or a combination thereof. With respect to cancer specifically, a therapeutically effective amount may, for example, result in the killing of cancer cells, reduce cancer cell counts, reduce tumor burden, eliminate tumors or metastasis, or reduce metastatic spread. A therapeutically effective amount may vary based on, for example, one or more of the following: the age and weight of the subject, the subject's overall health, the stage of the subject's disease, the route of administration, and prior or concomitant treatments.

Administration may comprise one or more (e.g., one, two, or three or more) dosing cycles.

In certain embodiments, the compounds contemplated by the present disclosure may be administered (e.g., orally, parenterally, etc.) at about 0.01 mg/kg to about 50 mg/kg, or about 1 mg/kg to about 25 mg/kg, of subject's body weight per day, one or more times a day, a week, or a month, to obtain the desired effect. In some embodiments, once daily or twice daily administration is contemplated. In some embodiments, a suitable weight-based dose of a compound contemplated by the present disclosure is used to determine a dose that is administered independent of a subject's body weight. In certain embodiments, the compounds of the present disclosure are administered (e.g., orally, parenterally, etc.) at fixed dosage levels of about 1 mg to about 1000 mg, particularly 1, 3, 5, 10, 15, 20, 25, 50, 75, 100, 150, 200, 250, 300, 400, 500, 600, 750, 800, 900, or 1000 mg, one or more times a day, a week, or a month, to obtain the desired effect.

In certain embodiments, the compound is contained in a “unit dosage form”. The phrase “unit dosage form” refers to physically discrete units, each unit containing a predetermined amount of the compound, either alone or in combination with one or more additional agents, sufficient to produce the desired effect. It will be appreciated that the parameters of a unit dosage form will depend on the particular agent and the effect to be achieved.

### Combination Therapy

The present disclosure contemplates the use of compounds disclosed herein alone or in combination with one or more additional therapy. Each additional therapy can be a therapeutic agent or another treatment modality. In embodiments comprising one or more additional therapeutic agents, each agent may target a different, but complementary, mechanism of action. The additional therapeutic agents can be small chemical molecules; macromolecules such as proteins, antibodies, peptibodies, peptides, DNA, RNA or fragments of such macromolecules; or cellular or gene therapies. Non-limiting examples of additional treatment modalities include surgical resection of a tumor, bone marrow transplant, radiation therapy, and photodynamic therapy. The use of a compound disclosed herein in combination with one or more additional therapies may have a synergistic therapeutic or prophylactic effect on the underlying disease, disorder, or condition. In addition to or alternatively, the combination therapy may allow for a dose reduction of one or more of the therapies, thereby ameliorating, reducing or eliminating adverse effects associated with one or more of the agents.

In embodiments comprising one or more additional treatment modality, the compound can be administered before, after or during treatment with the additional treatment modality. In embodiments comprising one or more additional therapeutic agents, the therapeutic agents used in such combination therapy can be formulated as a single composition or as separate compositions. If administered separately, each therapeutic agent in the combination can be given at or around the same time, or at different times. Furthermore, the therapeutic agents are administered “in combination” even if they have different forms of administration (e.g., oral capsule and intravenous), they are given at different dosing intervals, one therapeutic agent is given at a constant dosing regimen while another is titrated up, titrated down or discontinued, or each therapeutic agent in the combination is independently titrated up, titrated down, increased or decreased in dosage, or discontinued and/or resumed during a subject's course of therapy. If the combination is formulated as separate compositions, in some embodiments, the separate compositions are provided together in a kit.

### Cancer Therapies

The present disclosure contemplates the use of the compounds described herein in combination with one or more additional therapies useful in the treatment of cancer.

In some embodiments, one or more of the additional therapies is an additional treatment modality. Exemplary treatment modalities include but are not limited to surgical resection of a tumor, bone marrow transplant, radiation therapy, and photodynamic therapy.

In some embodiments, one or more of the additional therapies is a therapeutic agent. Exemplary therapeutic agents include chemotherapeutic agents, radiopharmaceuticals, hormone therapies, epigenetic modulators, ATP-adenosine axis-targeting agents, targeted therapies, signal transduction inhibitors, RAS signaling inhibitors, PI3K inhibitors, arginase inhibitors, HIF inhibitors, AXL inhibitors, PAK4 inhibitors, immunotherapeutic agents, cellular therapies, gene therapies, immune checkpoint inhibitors, and agonists of stimulatory or co-stimulatory immune checkpoints.

In some embodiments, one or more of the additional therapeutic agents is a chemotherapeutic agent. Examples of chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, pomalidomide, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pemetrexed, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel, nab paclitaxel, and docetaxel; chlorambucil; gemcitabine; 6-thioguanine; mercaptopurine; methotrexate; platinum and platinum coordination complexes such as cisplatin, carboplatin and oxaliplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; proteasome inhibitors such as bortezomib, carfilzomib and ixazomib; topoisomerase inhibitors such as irinotecan, topotecan, etoposide, mitoxantrone, teniposide; difluoromethylornithine (DMFO); retinoic acid; esperamicins; capecitabine; anthracyclines and pharmaceutically acceptable salts, acids or derivatives of any of the above. In certain embodiments, combination therapy comprises a chemotherapy regimen that includes one or more chemotherapeutic agents. In one embodiment, combination therapy comprises a chemotherapeutic regimen comprising one or more of FOLFOX (folinic acid, fluorouracil, and oxaliplatin), FOLFIRI (e.g., folinic acid, fluorouracil, and irinotecan), FOLFIRINOX (folinic acid, fluorouracil, irinotecan, and oxaliplatin), CAPOX (capecitabine and oxaliplatin), NALIRIFOX (fluorouracil, leucovorin, liposomal irinotecan, and oxaliplatin), .a taxoid (e.g., docetaxel, paclitaxel, nab-paclitaxel,etc.), a fluoropyrimidine-containing chemotherapeutic agent (e.g., fluorouracil, capecitabine, floxuridine), a platinum-containing chemotherapeutic agent, and/or gemcitabine.

In some embodiments, one or more of the additional therapeutic agents is a radiopharmaceutical. A radiopharmaceutical is a form of internal radiation therapy in which a source of radiation (i.e., one or more radionuclide) is put inside a subject's body. The radiation source can be in solid or liquid form. Non-limiting examples of radiopharmaceuticals include sodium iodide I-131, radium-223 dichloride, lobenguane iodine-131, radioiodinated vesicles (e.g., saposin C-dioleoylphosphatidylserine (SapC-DOPS) nanovesicles), various forms of brachytherapy, and various forms of targeted radionuclides. Targeted radionuclides comprise a radionuclide associated (e.g., by covalent or ionic interactions) with a molecule (“a targeting agent”) that specifically binds to a target on a cell, typically a cancer cell or an immune cell. The targeting agent may be a small molecule, a saccharide (inclusive of oligosaccharides and polysaccharides), an antibody, a lipid, a protein, a peptide, a non-natural polymer, or an aptamer. In some embodiments, the targeting agent is a saccharide (inclusive of oligosaccharides and polysaccharides), a lipid, a protein, or a peptide and the target is a tumor-associated antigen (enriched but not specific to a cancer cell), a tumor-specific antigen (minimal to no expression in normal tissue), or a neo-antigen (an antigen specific to the genome of a cancer cell generated by non-synonymous mutations in the tumor cell genome). In some embodiments, the targeting agent is an antibody and the target is a tumor-associated antigen (i.e., an antigen enriched but not specific to a cancer cell), a tumor-specific antigen (i.e., an antigen with minimal to no expression in normal tissue), or a neo-antigen (i.e., an antigen specific to the genome of a cancer cell generated by non-synonymous mutations in the tumor cell genome). Non-limiting examples of targeted radionuclides include radionuclides attached to: somatostatin or peptide analogs thereof (e.g., 177Lu-Dotatate, etc.); prostate specific membrane antigen or peptide analogs thereof (e.g., 177Lu-PSMA-617, 225Ac-PSMA-617, 177Lu-PSMA-I&T, 177Lu-MIP-1095, etc.); a receptor's cognate ligand, peptide derived from the ligand, or variants thereof (e.g., 188Re-labeled VEGF₁₂₅₋₁₃₆ or variants thereof with higher affinity to VEGF receptor, etc.); antibodies targeting tumor antigens (e.g., 131I-tositumomab, 90Y-ibritumomab tiuxetan, CAM-H2-I131 (Precirix NV), I131-omburtamab, etc.).

In some embodiments, one or more of the additional therapeutic agents is a hormone therapy. Hormone therapies act to regulate or inhibit hormonal action on tumors. Examples of hormone therapies include, but are not limited to: selective estrogen receptor degraders such as fulvestrant, giredestrant, SAR439859, RG6171, AZD9833, rintodestrant, ZN-c5, LSZ102, D-0502, LY3484356, SHR9549; selective estrogen receptor modulators such as tamoxifen, raloxifene, 4-hydroxytamoxifen, trioxifene, keoxifene, toremifene; aromatase inhibitors such as anastrozole, exemestane, letrozole and other aromatase inhibiting 4(5)-imidazoles; gonadotropin-releasing hormone agonists such as nafarelin, triptorelin, goserelin; gonadotropin-releasing hormone antagonists such as degarelix; antiandrogens such as abiraterone, enzalutamide, apalutamide, darolutamide, flutamide, nilutamide, bicalutamide, leuprolide; 5α-reductase inhibitors such as finasteride, dutasteride; and the like. In certain embodiments, combination therapy comprises administration of a hormone or related hormonal agent. In one embodiment, combination therapy comprises administration of enzalutamide.

In some embodiments, one or more of the additional therapeutic agents is an epigenetic modulator. An epigenetic modulator alters an epigenetic mechanism controlling gene expression, and may be, for example, an inhibitor or activator of an epigenetic enzyme. Non-limiting examples of epigenetic modulators include DNA methyltransferase (DNMT) inhibitors, hypomethylating agents, and histone deacetylase (HDAC) inhibitors. In one or more embodiments, the compounds according to this disclosure are combined with DNA methyltransferase (DNMT) inhibitors or hypomethylating agents. Exemplary DNMT inhibitors include decitabine, zebularine and azacitadine. In one or more embodiments, combinations of the compounds according to this disclosure with a histone deacetylase (HDAC) inhibitor is also contemplated. Exemplary HDAC inhibitors include vorinostat, givinostat, abexinostat, panobinostat, belinostat and trichostatin A.

In some embodiments, one or more of the additional therapeutic agents is an ATP-adenosine axis-targeting agent. ATP-adenosine axis-targeting agents alter signaling mediated by adenine nucleosides and nucleotides (e.g., adenosine, AMP, ADP, ATP), for example by modulating the level of adenosine or targeting adenosine receptors. Adenosine and ATP, acting at different classes of receptors, often have opposite effects on inflammation, cell proliferation and cell death. For instance, ATP and other adenine nucleotides have antitumor effects via activation of the PS2Y1 receptor subtype, while accumulation of adenosine in the tumor microenvironment has been shown to inhibit the antitumor function of various immune cells and to augment the immunosuppressive activity of myeloid and regulatory T cells by binding to cell surface adenosine receptors. In certain embodiments, an ATP-adenosine axis-targeting agent is an inhibitor of an ectonucleotidase involved in the conversion of ATP to adenosine or an antagonist of adenosine receptor. Ectonucleotidases involved in the conversion of ATP to adenosine include the ectonucleoside triphosphate diphosphohydrolase 1 (ENTPD1, also known as CD39 or Cluster of Differentiation 39) and the ecto-5′-nucleotidase (NT5E or 5NT, also known as CD73 or Cluster of Differentiation 73). Exemplary small molecule CD73 inhibitors include CB-708, ORIC-533, LY3475070 and quemliclustat. Exemplary anti-CD39 and anti-CD73 antibodies include ES002023, TTX-030, IPH-5201, SRF-617, CPI-006, oleclumab (MEDI9447), NZV930, IPH5301, GS-1423, uliledlimab (TJD5, TJ004309), AB598, and BMS-986179. In one embodiment, the present disclosure contemplates combination of the compounds described herein with a CD73 inhibitor such as those described in WO 2017/120508, WO 2018/067424, WO 2018/094148, and WO 2020/046813. In further embodiments, the CD73 inhibitor is quemliclustat (AB680). Adenosine can bind to and activate four different G-protein coupled receptors: A₁R, A_{2A}R, A_{2B}R, and A₃R. A₂R antagonists include etrumadenant, inupadenant, taminadenant, caffeine citrate, NUV-1182, TT-702, DZD-2269, INCB-106385, EVOEXS-21546, AZD-4635, imaradenant, RVU-330, ciforadenant, PBF-509, PBF-999, PBF-1129, and CS-3005. In some embodiments, the present disclosure contemplates the combination of the compounds described herein with an A2_{A}R antagonist, an A2_{B}R antagonist, or an antagonist of A2_{A}R and A2_{B}R. In some embodiments, the present disclosure contemplates the combination of the compounds described herein with the adenosine receptor antagonists described in WO 2018/136700, WO 2018/204661, WO 2018/213377, or WO 2020/023846. In one embodiment, the adenosine receptor antagonist is etrumadenant.

In some embodiments, one or more of the additional therapeutic agents is a targeted therapy. In one aspect, a targeted therapy may comprise a targeting agent and a drug. The drug may be a chemotherapeutic agent, a radionuclide, a hormone therapy, or another small molecule drug attached to a targeting agent. The targeting agent may be a small molecule, a saccharide (inclusive of oligosaccharides and polysaccharides), an antibody, a lipid, a protein, a peptide, a non-natural polymer, or an aptamer. In some embodiments, the targeting agent is a saccharide (inclusive of oligosaccharides and polysaccharides), a lipid, a protein, or a peptide and the target is a tumor-associated antigen (enriched but not specific to a cancer cell), a tumor-specific antigen (minimal to no expression in normal tissue), or a neo-antigen (an antigen specific to the genome of a cancer cell generated by non-synonymous mutations in the tumor cell genome). In some embodiments, the targeting agent is an antibody and the target is a tumor-associated antigen, a tumor-specific antigen, or a neo-antigen. In some embodiments, the targeted therapy is an antibody-drug conjugate comprising an antibody and a drug, wherein the antibody specifically binds to HER2, HER3, nectin-4, or Trop-2. Specific examples of a targeted therapy comprising an antibody and a drug include but are not limited to patritumab deruxtecan, sacituzumab govitecan-hziy, telisotuzumab vedotin, and trastuzumab deruxtecan. Specific examples include but are not limited to patritumab deruxtecan and telisotuzumab vedotin. In other aspects, a targeted therapy may inhibit or interfere with a specific protein that helps a tumor grow and/or spread. Non-limiting examples of such targeted therapies include signal transduction inhibitors, RAS signaling inhibitors, inhibitors of oncogenic transcription factors, activators of oncogenic transcription factor repressors, angiogenesis inhibitors, immunotherapeutic agents, ATP-adenosine axis-targeting agents, AXL inhibitors, PARP inhibitors, PAK4 inhibitors, PI3K inhibitors, HIF-2α inhibitors, CD39 inhibitors, CD73 inhibitors, A2R antagonists, TIGIT antagonists, and PD-1 antagonists. ATP-adenosine axis-targeting agents are described above, while other agents are described in further detail below.

In some embodiments, one or more of the additional therapeutic agents is a signal transduction inhibitor. Signal transduction inhibitors are agents that selectively inhibit one or more steps in a signaling pathway. Signal transduction inhibitors (STIs) contemplated by the present disclosure include but are not limited to: (i) BCR-ABL kinase inhibitors (e.g., imatinib); (ii) epidermal growth factor receptor tyrosine kinase inhibitors (EGFR TKIs), including small molecule inhibitors (e.g., CLN-081, gefitinib, erlotinib, afatinib, icotinib, and osimertinib), and anti-EGFR antibodies; (iii) inhibitors of the human epidermal growth factor (HER) family of transmembrane tyrosine kinases, e.g., HER-2/neu receptor inhibitors (e.g., trastuzumab) and HER-3 receptor inhibitors; (iv) vascular endothelial growth factor receptor (VEGFR) inhibitors including small molecule inhibitors (e.g., axitinib, sunitinib and sorafenib), VEGF kinase inhibitors (e.g., lenvatinib, cabozantinib, pazopanib, tivozanib, XL092, etc.) and anti-VEGF antibodies (e.g., bevacizumab), and anti-VEGFR antibodies (e.g., ramucirumab); (v) inhibitors of AKT family kinases or the AKT pathway (e.g., rapamycin); (vi) inhibitors of mTOR, such as, for example, everolimus, sirolimus, temsirolimus; (vii) inhibitors of serine/threonine-protein kinase B-Raf (BRAF), such as, for example, vemurafenib, dabrafenib and encorafenib; (viii) inhibitors of rearranged during transfection (RET), including, for example, selpercatinib and pralsetinib; (ix) tyrosine-protein kinase Met (MET) inhibitors (e.g., tepotinib, tivantinib, cabozantinib and crizotinib); (x) anaplastic lymphoma kinase (ALK) inhibitors (e.g., ensartinib, ceritinib, lorlatinib, crizotinib, and brigatinib); (xi) inhibitors of the RAS signaling pathway (e.g., inhibitors of KRAS, HRAS, RAF, MEK, ERK) as described elsewhere herein; (xii) FLT-3 inhibitors (e.g., gilteritinib);(xiii) inhibitors of Trop-2; (xiv) inhibitors of the JAK/STAT pathway, e.g., JAK inhibitors including tofacitinib and ruxolitinib, or STAT inhibitors such as napabucasin; (xv) inhibitors of NF-κB; (xvi) cell cycle kinase inhibitors (e.g., flavopiridol); (xvii) phosphatidyl inositol kinase (PI3K) inhibitors; (xiii) protein kinase B (AKT) inhibitors (e.g., capivasertib, miransertib); (xviii) platelet-derived growth factor receptor (PDGFR) inhibitors (e.g., imatinib, sunitinib, regorafenib, avapritinib, lenvatinib, nintedanib, famitinib, ponatinib, axitinib, repretinib, etc.); (xix) insulin-like growth factor receptor (IGFR) inhibitors (e.g., erlotinib, afatinib, gefitinib, osimertinib, dacomitinib); (xx) fibroblast growth factor receptor (FGFR) inhibitors (e.g., futibatinib, erdafitinib, pemigatinib); and (xxi) receptor tyrosine kinase KIT inhibitors (e.g., imatinib, sorafenib, sunitinib, masitinib, repretinib, avapritinib). In one or more embodiments, the additional therapeutic agent comprises an inhibitor of EGFR, VEGFR, HER-2, HER-3, BRAF, RET, MET, ALK, RAS (e.g., KRAS, MEK, ERK), FLT-3, JAK, STAT, NF-κB, PI3K, AKT, FGFR, KIT, or any combinations thereof. In some embodiments, one or more of the additional therapeutic agents is a RAS signaling inhibitor. Oncogenic mutations in the RAS family of genes, e.g., HRAS, KRAS, and NRAS, are associated with a variety of cancers. For example, mutations of G12C, G12D, G12V, G12A, G13D, Q61H, G13C and G12S, among others, in the KRAS family of genes have been observed in multiple tumor types. Direct and indirect inhibition strategies have been investigated for the inhibition of mutant RAS signaling. Indirect inhibitors target effectors other than RAS in the RAS signaling pathway, and include, but are not limited to, inhibitors of RAF, MEK, ERK, PI3K, PTEN, SOS (e.g., SOS1), mTORC1, SHP2 (PTPN11), and AKT. Non-limiting examples of indirect inhibitors under development include RMC-4630, RMC-5845, RMC-6291, RMC-6236, JAB-3068, JAB-3312, TNO155, RLY-1971, BI1701963. Direct inhibitors of RAS mutants have also been explored, and generally target the KRAS-GTP complex or the KRAS-GDP complex. Exemplary direct RAS inhibitors under development include, but are not limited to, sotorasib (AMG510), adagrasib (MRTX849), mRNA-5671 and ARS1620. In some embodiments, the one or more RAS signaling inhibitors are selected from the group consisting of RAF inhibitors, MEK inhibitors, ERK inhibitors, PI3K inhibitors, PTEN inhibitors, SOS1 inhibitors, mTORC1 inhibitors, SHP2 inhibitors, and AKT inhibitors. In other embodiments the one or more RAS signaling inhibitors directly inhibit RAS mutants.

In some embodiments one or more of the additional therapeutic agents is an inhibitor of a phosphatidylinositol 3-kinase (PI3K), particularly an inhibitor of the PI3Kγ isoform. PI3Kγ inhibitors can stimulate an anti-cancer immune response through the modulation of myeloid cells, such as by inhibiting suppressive myeloid cells, dampening immune-suppressive tumor-infiltrating macrophages or by stimulating macrophages and dendritic cells to make cytokines that contribute to effective T cell responses thereby decreasing cancer development and spread. Exemplary PI3Kγ inhibitors include copanlisib, duvelisib, AT-104, ZX-101, tenalisib, eganelisib, SF-1126, AZD3458, and pictilisib. In some embodiments, the compounds according to this disclosure are combined with one or more PI3Kγ inhibitors described in WO 2020/0247496A1.

In some embodiments, one or more of the additional therapeutic agents is an inhibitor of arginase. Arginase has been shown to be either responsible for or participate in inflammation-triggered immune dysfunction, tumor immune escape, immunosuppression and immunopathology of infectious disease. Exemplary arginase compounds include CB-1158 and OAT-1746. In some embodiments, the compounds according to this disclosure are combined with one or more arginase inhibitors described in WO/2019/173188 and WO 2020/102646.

In some embodiments, one or more of the additional therapeutic agents is an inhibitor of an oncogenic transcription factor or an activator of an oncogenic transcription factor repressor. Suitable agents may act at the expression level (e.g., RNAi, siRNA, etc.), through physical degradation, at the protein/protein level, at the protein/DNA level, or by binding in an activation/inhibition pocket. Non-limiting examples include inhibitors of one or more subunit of the MLL complex (e.g., HDAC, DOT1L, BRD4, Menin, LEDGF, WDR5, KDM4C (JMJD2C) and PRMT1), inhibitors of hypoxia-inducible factor (HIF) transcription factor, and the like.

In some embodiments, one or more of the additional therapeutic agents is an inhibitor of a hypoxia-inducible factor (HIF) transcription factor, particularly HIF-2α. Exemplary HIF-2α inhibitors include belzutifan, ARO-HIF2, PT-2385, AB521, NKT-2152, DFF332, and those described in WO 2021113436, WO 2021188769, and WO 2023077046. In some embodiments, the HIF-2α inhibitor is AB521.

In some embodiments, one or more of the additional therapeutic agents is an inhibitor of anexelekto (AXL). The AXL signaling pathway is associated with tumor growth and metastasis and is believed to mediate resistance to a variety of cancer therapies. There are a variety of AXL inhibitors under development that also inhibit other kinases in the TAM family (i.e., TYRO3, MERTK), as well as other receptor tyrosine kinases including MET, FLT3, RON and AURORA, among others. Exemplary multikinase inhibitors include sitravatinib, rebastinib, glesatinib, gilteritinib, merestinib, cabozantinib, foretinib, BMS777607, LY2801653, S49076, and RXDX-106. AXL specific inhibitors have also been developed, e.g., small molecule inhibitors including DS-1205, SGI-7079, SLC-391, dubermatinib, bemcentinib, DP3975, and AB801; anti-AXL antibodies such as ADCT-601; and antibody drug conjugates (ADCs) such as BA3011. Another strategy to inhibit AXL signaling involves targeting AXL's ligand, GAS6. For example, batiraxcept is under development as is a Fc fusion protein that binds the GAS6 ligand thereby inhibiting AXL signaling. In some embodiments, the compounds according to this disclosure are combined with one or more AXL inhibitors described in WO2022246177, WO2022246179, or WO2024006726. In some embodiments, the AXL inhibitor is AB801.

In some embodiments, one or more of the additional therapeutic agents is an inhibitor of p21-activated kinase 4 (PAK4). PAK4 overexpression has been shown across a variety of cancer types, notably including those resistant to PD-1 therapies.

In some embodiments, one or more of the additional therapeutic agents is (i) an agent that inhibits the enzyme poly (ADP-ribose) polymerase (e.g., olaparib, niraparib and rucaparib, etc.); (ii) an inhibitor of the Bcl-2 family of proteins (e.g., venetoclax, navitoclax, etc.); (iii) an inhibitor of MCL-1; (iv) an inhibitor of the CD47-SIRPα pathway (e.g., an anti-CD47 antibody); (v) an isocitrate dehydrogenase (IDH) inhibitor, e.g., IDH-1 or IDH-2 inhibitor (e.g., ivosidenib, enasidenib, etc.).

In some embodiments, one or more of the additional therapeutic agents is an immunotherapeutic agent. Immunotherapeutic agents treat a disease by stimulating or suppressing the immune system. Immunotherapeutic agents useful in the treatment of cancers typically elicit or amplify an immune response to cancer cells. Non-limiting examples of suitable immunotherapeutic agents include: immunomodulators; cellular immunotherapies; vaccines; gene therapies; ATP-adenosine axis-targeting agents; immune checkpoint modulators; and certain signal transduction inhibitors. ATP-adenosine axis-targeting agents and signal transduction inhibitors are described above. Immunomodulators, cellular immunotherapies, vaccines, gene therapies, and immune checkpoint modulators are described further below.

In some embodiments, one or more of the additional therapeutic agents is an immunotherapeutic agent, more specifically a cytokine or chemokine, such as, IL-1, IL-2, IL-12, IL-18, ELC/CCL19, SLC/CCL21, MCP-1, IL-4, TNF, IL-15, MDC, IFNα, IFNβ, IFNγ, M-CSF, IL-3, GM-CSF, IL-13, and anti-IL-10; bacterial lipopolysaccharides (LPS); an organic or inorganic adjuvant that activates antigen-presenting cells and promote the presentation of antigen epitopes on major histocompatibility complex molecules agonists including, but not limited to Toll-like receptor (TLR) agonists, antagonists of the mevalonate pathway, agonists of STING; indoleamine 2,3-dioxygenase 1 (IDO1) inhibitors and immune-stimulatory oligonucleotides, as well as other T cell adjuvants.

In some embodiments, one or more of the additional therapeutic agents is an immunotherapeutic agent, more specifically a cellular therapy. Cellular therapies are a form of treatment in which viable cells are administered to a subject. In certain embodiments, one or more of the additional therapeutic agents is a cellular immunotherapy that activates or suppresses the immune system. Cellular immunotherapies useful in the treatment of cancers typically elicit or amplify an immune response. The cells can be autologous or allogenic immune cells (e.g., monocytes, macrophages, dendritic cells, NK cells, T cells, etc.) collected from one or more subject. Alternatively, the cells can be “(re)programmed” allogenic immune cells produced from immune precursor cells (e.g., lymphoid progenitor cells, myeloid progenitor cells, common dendritic cell precursor cells, stem cells, induced pluripotent stem cells, etc.). In some embodiments, such cells may be an expanded subset of cells with distinct effector functions and/or maturation markers (e.g., adaptive memory NK cells, tumor infiltrating lymphocytes, immature dendritic cells, monocyte-derived dendritic cells, plasmacytoid dendritic cells, conventional dendritic cells (sometimes referred to as classical dendritic cells), M1 macrophages, M2 macrophages, etc.), may be genetically modified to target the cells to a specific antigen and/or enhance the cells' anti-tumor effects (e.g., engineered T cell receptor (TCR) cellular therapies, chimeric antigen receptor (CAR) cellular therapies, lymph node homing of antigen-loaded dendritic cells, etc.), may be engineered to express of have increased expression of a tumor-associated antigen, or may be any combination thereof. Non-limiting types of cellular therapies include CAR-T cell therapy, CAR-NK cell therapy, TCR therapy, and dendritic cell vaccines. Exemplary cellular immunotherapies include sipuleucel-T, tisagenlecleucel, lisocabtagene maraleucel, idecabtagene vicleucel, brexucabtagene autoleucel, and axicabtagene ciloleucel, as well as CTX110, JCAR015, JCAR017, MB-CART19.1, MB-CART20.1, MB-CART2019.1, UniCAR02-T-CD123, BMCA-CAR-T, JNJ-68284528, BNT211, and NK-92/5.28.z.

In some embodiments, one or more of the additional therapeutic agents is an immunotherapeutic agent, more specifically a gene therapy. Gene therapies comprise recombinant nucleic acids administered to a subject or to a subject's cells ex vivo in order to modify the expression of an endogenous gene or to result in heterologous expression of a protein (e.g., small interfering RNA (siRNA) agents, double-stranded RNA (dsRNA) agents, micro RNA (miRNA) agents, viral or bacterial gene delivery, etc.), as well as gene editing therapies that may or may not comprise a nucleic acid component (e.g., meganucleases, zinc finger nucleases, TAL nucleases, CRISPR/Cas nucleases, etc.), oncolytic viruses, and the like. Non-limiting examples of gene therapies that may be useful in cancer treatment include rAd-p53, rAD5-H101, talimogene laherparepvec, Mx-dnG1, ARO-HIF2 (Arrowhead), quaratusugene ozeplasmid (Immunogene), CTX110 (CRISPR Therapeutics), CTX120 (CRISPR Therapeutics), and CTX130 (CRISPR Therapeutics).

In some embodiments, one or more of the additional therapeutic agents is an immunotherapeutic agent, more specifically an agent that modulates an immune checkpoint. Immune checkpoints are a set of inhibitory and stimulatory pathways that directly affect the function of immune cells (e.g., B cells, T cells, NK cells, etc.). Immune checkpoints engage when proteins on the surface of immune cells recognize and bind to their cognate ligands. The present invention contemplates the use of compounds described herein in combination with agonists of stimulatory or co-stimulatory pathways and/or antagonists of inhibitory pathways. Agonists of stimulatory or co-stimulatory pathways and antagonists of inhibitory pathways may have utility as agents to overcome distinct immune suppressive pathways within the tumor microenvironment, inhibit T regulatory cells, reverse/prevent T cell anergy or exhaustion, trigger innate immune activation and/or inflammation at tumor sites, or combinations thereof.

In some embodiments, one or more of the additional therapeutic agents is an immune checkpoint inhibitor. As used herein, the term “immune checkpoint inhibitor” refers to an antagonist of an inhibitory or co-inhibitory immune checkpoint. The terms “immune checkpoint inhibitor”, “checkpoint inhibitor” and “CPI” may be used herein interchangeably. Immune checkpoint inhibitors may antagonize an inhibitory or co-inhibitory immune checkpoint by interfering with receptor-ligand binding and/or altering receptor signaling. Examples of immune checkpoints (ligands and receptors), some of which are selectively upregulated in various types of cancer cells, that can be antagonized include PD-1 (programmed cell death protein 1); PD-L1 (PD1 ligand); BTLA (B and T lymphocyte attenuator); CTLA-4 (cytotoxic T-lymphocyte associated antigen 4); TIM-3 (T cell immunoglobulin and mucin domain containing protein 3); LAG-3 (lymphocyte activation gene 3); TIGIT (T cell immunoreceptor with Ig and ITIM domains); CD276 (B7-H3), PD-L2, Galectin 9, CEACAM-1, CD69, Galectin-1, CD113, GPR56, VISTA, 2B4, CD48, GARP, PD1H, LAIR1, TIM-1, and TIM-4, and Killer Inhibitory Receptors, which can be divided into two classes based on their structural features: i) killer cell immunoglobulin-like receptors (KIRs), and ii) C-type lectin receptors (members of the type II transmembrane receptor family). Also contemplated are other less well-defined immune checkpoints that have been described in the literature, including both receptors (e.g., the 2B4 (also known as CD244) receptor) and ligands (e.g., certain B7 family inhibitory ligands such B7-H3 (also known as CD276) and B7-H4 (also known as B7-S1, B7x and VCTN1)). [See Pardoll, (April 2012) Nature Rev. Cancer 12:252-64].

In some embodiments, an immune checkpoint inhibitor is a CTLA-4 antagonist. In further embodiments, the CTLA-4 antagonist can be an antagonistic CTLA-4 antibody. Suitable antagonistic CTLA-4 antibodies include, for example, monospecific antibodies such as ipilimumab or tremelimumab, as well as bispecific antibodies such as MEDI5752 and KN046.

In some embodiments, an immune checkpoint inhibitor is a PD-1 antagonist. In further embodiments, the PD-1 antagonist can be an antagonistic PD-1 antibody, small molecule or peptide. Suitable antagonistic PD-1 antibodies include, for example, monospecific antibodies such as balstilimab, budigalimab, camrelizumab, cosibelimab, dostarlimab, cemiplimab, ezabenlimab, MEDI-0680 (AMP-514; WO2012/145493), nivolumab, pembrolizumab, pidilizumab (CT-011), pimivalimab, retifanlimab, sasanlimab, spartalizumab, sintilimab, tislelizumab, toripalimab, and zimberelimab; as well as bi-specific antibodies such as LY3434172, IBI321, ivonescimab, rilvegostomig, tebotelimab, and tobemstomig. In still further embodiments, the PD-1 antagonist can be a recombinant protein composed of the extracellular domain of PD-L2 (B7-DC) fused to the Fc portion of IgGI (AMP-224). In certain embodiments, an immune checkpoint inhibitor is zimberelimab.

In some embodiments, an immune checkpoint inhibitor is a PD-L1 antagonist. In further embodiments, the PD-L1 antagonist can be an antagonistic PD-L1 antibody. Suitable antagonistic PD-L1 antibodies include, for example, monospecific antibodies such as avelumab, atezolizumab, durvalumab, BMS-936559, and envafolimab as well as bi-specific antibodies such as LY3434172 and KN046.

In some embodiments, an immune checkpoint inhibitor is a TIGIT antagonist. In further embodiments, the TIGIT antagonist can be an antagonistic TIGIT antibody. Suitable antagonistic anti-TIGIT antibodies include monospecific antibodies such as AGEN1327, AB308 (WO2021247591), BMS 986207, COM902, domvanalimab, belrestotug, etigilimab, IBI-929, JS006, dargistotug, ociperlimab, SEA-TGT, tiragolumab, vibostolimab; as well as bi-specific antibodies such as AGEN1777 and rilvegostomig. In certain embodiments, an immune checkpoint inhibitor is an antagonistic anti-TIGIT antibody disclosed in WO2017152088 or WO2021247591. In certain embodiments, an immune checkpoint inhibitor is domvanalimab or AB308.

In some embodiments, an immune checkpoint inhibitor is a LAG-3 antagonist. In further embodiments, the LAG-3 antagonist can be an antagonistic LAG-3 antibody. Suitable antagonistic LAG-3 antibodies include, for example, BMS-986016 (WO10/19570, WO14/08218), or IMP-731 or IMP-321 (WO08/132601, WO09/44273).

In certain embodiments, an immune checkpoint inhibitor is a B7-H3 antagonist. In further embodiments, the B7-H3 antagonist is an antagonistic B7-H3 antibody. Suitable antagonist B7-H3 antibodies include, for example, enoblituzumab (WO11/109400), omburtumab, DS-7300a, ABBV-155, and SHR-A1811.

In some embodiments, an immune checkpoint inhibitor is a TIM-3 antagonist. In further embodiments, the TIM-3 antagonist can be an antagonistic TIM-3 antibody. Suitable antagonistic TIM-3 antibodies include, for example, sabatolimab, BMS-986258, and RG7769/RO7121661.

In some embodiments, one or more of the additional therapeutic agents activates a stimulatory or co-stimulatory immune checkpoint. Examples of stimulatory or co-stimulatory immune checkpoints (ligands and receptors) include B7-1, B7-2, CD28, 4-1BB (CD137), 4-1BBL, ICOS, ICOS-L, OX40, OX40L, GITR, GITRL, CD70, CD27, CD40, DR3 and CD2.

In some embodiments, an agent that activates a stimulatory or co-stimulatory immune checkpoint is a CD137 (4-1BB) agonist. In further embodiments, the CD137 agonist can be an agonistic CD137 antibody. Suitable CD137 antibodies include, for example, urelumab and utomilumab (WO12/32433). In some embodiments, an agent that activates a stimulatory or co-stimulatory immune checkpoint is a GITR agonist. In further embodiments, the GITR agonist can be an agonistic GITR antibody. Suitable GITR antibodies include, for example, BMS-986153, BMS-986156, TRX-518 (WO06/105021, WO09/009116) and MK-4166 (WO11/028683). In some embodiments, an agent that activates a stimulatory or co-stimulatory immune checkpoint is an OX40 agonist. In further embodiments, the OX40 agonist can be an agonistic OX40 antibody. Suitable OX40 antibodies include, for example, MEDI-6383, MEDI-6469, MEDI-0562, PF-04518600, GSK3174998, BMS-986178, and MOXR0916. In some embodiments, an agent that activates a stimulatory or co-stimulatory immune checkpoint is a CD40 agonist. In further embodiments, the CD40 agonist can be an agonistic CD40 antibody. In some embodiments, an agent that activates a stimulatory or co-stimulatory immune checkpoint is a CD27 agonist. In further embodiments, the CD27 agonist can be an agonistic CD27 antibody. Suitable CD27 antibodies include, for example, varlilumab.

In some embodiments, one or more of the additional therapies is an immunotherapeutic agent, more specifically an intracellular signaling molecule that influences immune cell function. For example, one or more of the additional therapies may be an inhibitor of hematopoietic progenitor kinase 1 (HPK1). HPK1 is serine/threonine kinase that functions as a negative regulator of activation signals generated by the T cell antigen receptor. As another example, one or more of the additional therapies may be an inhibitor of diacylglycerol kinase (DGK). In some embodiments, the inhibitor is a small molecule. Non-limiting examples of small molecule HPK1 inhibitors in clinical development include NDI-101150, PRJ1-3024, PF-07265028, GRC 54276, CFI-402411 and BGB-15025. Non-limiting examples of small molecule DGK inhibitors include ASP1570, BAY2965501.

In some embodiments, one or more of the additional therapeutic agents is an agent that inhibits or depletes immune-suppressive immune cells. For example, to inhibit or deplete immunosuppressive macrophages or monocytes the agent may be CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264), or CSF-1R antagonists disclosed in WO14/036357. In another example, to inhibit or deplete regulatory T cells (Tregs) the agent may be an anti-CD25 antibody or immunotoxin targeting CD25.

In some embodiments, each additional therapeutic agent can independently be a chemotherapeutic agent, a radiopharmaceutical, a hormone therapy, an epigenetic modulator, a targeted agent, an immunotherapeutic agent, a cellular therapy, or a gene therapy. For example, in one embodiment, the present disclosure contemplates the use of the compounds described herein in combination with one or more chemotherapeutic agent and optionally one or more additional therapeutic agents, wherein each additional therapeutic agent is independently a radiopharmaceutical, a hormone therapy, a targeted agent, an immunotherapeutic agent, a cellular therapy, or a gene therapy. In another embodiment, the present disclosure contemplates the use of the compounds described herein in combination with one or more chemotherapeutic agent and optionally one or more additional therapeutic agents, wherein each additional therapeutic agent is independently a targeted agent, an immunotherapeutic agent, or a cellular therapy. In another embodiment, the present disclosure contemplates the use of the compounds described herein in combination with one or more immunotherapeutic agents and optionally one or more additional therapeutic agent, wherein each additional therapeutic agent is independently a radiopharmaceutical, a hormone therapy, a targeted agent, a chemotherapeutic agent, a cellular therapy, or a gene therapy. In another embodiment, the present disclosure contemplates the use of the compounds described herein in combination with one or more immunotherapeutic agents and optionally one or more additional therapeutic agents, wherein each additional therapeutic agent is independently a chemotherapeutic agent, a targeted agent, or a cellular therapy. In another embodiment, the present disclosure contemplates the use of the compounds described herein in combination with one or more immune checkpoint inhibitors and/or one or more ATP-adenosine axis-targeting agents, and optionally one or more additional therapeutic agents, wherein each additional therapeutic agent is independently a chemotherapeutic agent, a targeted agent, an immunotherapeutic agent, or a cellular therapy. In further embodiments of the above (a) the targeted agent can be a PI3K inhibitor, an arginase inhibitor, a HIF2α inhibitor, an AXL inhibitor, or a PAK4 inhibitor; (b) the immunotherapeutic agent is an ATP-adenosine axis-targeting agent or an immune checkpoint inhibitor; (c) the ATP-adenosine axis-targeting agent is an A2_{A}R and/or A2_{B}R antagonist, a CD73 inhibitor, or a CD39 inhibitor; (d) the ATP-adenosine axis-targeting agent is etrumadenant, quemliclustat, or AB598; (e) the immunotherapeutic agent is an anti-PD-1 antagonist antibody or an anti-TIGIT antagonist antibody; (f) the immunotherapeutic agent is zimberelimab, domvanalimab, or AB308; or (g) any combination thereof. In still further embodiments of the above, the present disclosure contemplates the use of the compounds described herein in combination with domvanalimab, etrumadenant, quemliclustat, zimberelimab, AB308, AB521, AB598, AB610, AB801 or any combination thereof.

Selection of the additional therapeutic agent(s) may be informed by current standard of care for a particular cancer and/or mutational status of a subject's cancer and/or stage of disease. Detailed standard of care guidelines are published, for example, by National Comprehensive Cancer Network (NCCN). See, for instance, NCCN Colon Cancer v1.2022, NCCN Hepatobiliary Cancer v1.2022, NCCN Kidney Cancer, v3.2022, NCCN NSCLC v3.2022, NCCN Pancreatic Adenocarcinoma v1.2022, NCCN Esophageal and Esophagogastric Junction Cancers v2.2022, NCCN Gastric Cancer v2.2022, Cervical Cancer v1.2022, Ovarian Cancer/Fallopian Tube Cancer/Primary Peritoneal Cancer v1.2022.

### CERTAIN EMBODIMENTS OF THE DISCLOSURE

Embodiment 1: A method of treating a disease, disorder, or condition mediated at least in part by Cbl-b in a subject in need thereof, the method comprising administering to the subject a therapeutically effective amount of the compound according to this disclosure, or pharmaceutically acceptable salt thereof, or a pharmaceutical composition of comprising a compound according to this disclosure and a pharmaceutically acceptable excipient.

Embodiment 2: The method of embodiment 1, wherein the compound or pharmaceutical composition is administered in a therapeutically effective amount to inhibit Cbl-b.

Embodiment 3: The method of embodiment 1 or 2, wherein the disease, disorder, or condition is cancer.

Embodiment 4: The method of embodiment 3, wherein the cancer is cancer of the genitourinary tract (e.g., gynecologic, bladder, kidney, renal cell, penile, prostate, or testicular), breast, gastrointestinal tract (e.g., esophagus, oropharynx, stomach, small or large intestines, colon, or rectum), bone, bone marrow, skin (e.g., melanoma), head and neck, liver, gall bladder, bile ducts, heart, lung, pancreas, salivary gland, adrenal gland, thyroid, brain (e.g., gliomas), ganglia, central nervous system (CNS), peripheral nervous system (PNS), the hematopoietic system (i.e., hematological malignancies), or the immune system (e.g., spleen or thymus), or any combination thereof.

Embodiment 5: The method of embodiment 3, wherein the cancer is breast cancer, genitourinary cancer, gastrointestinal cancer, lung cancer, skin cancer, or a combination thereof.

Embodiment 6: The method of embodiment 3, wherein the cancer is a hematological malignancy, optionally selected from leukemias, lymphomas and myelomas.

Embodiment 7: The method of any one of embodiments 1-6, further comprising administering at least one additional therapeutic agent to the subject.

Embodiment 8: The method of embodiment 7, wherein said at least one additional therapeutic agent comprises one or more agents independently selected from the groups consisting of immune checkpoint inhibitors, agents that target the extracellular production of adenosine, inhibitors of HIF (e.g., a HIF-2α inhibitor), kinase inhibitors, radiation therapy, and chemotherapeutic agents.

Embodiment 9: The method of embodiment 8, wherein said at least one additional therapeutic agent comprises one or more immune checkpoint inhibitors that antagonizes at least one of PD-1, PD-L1, BTLA, LAG-3, a B7 family member, TIM-3, TIGIT, or CTLA-4.

Embodiment 10: The method of embodiment 9, wherein said one or more immune checkpoint inhibitors comprise an immune checkpoint inhibitor that antagonizes PD-1 or PD-L1.

Embodiment 11: The method of embodiment 10, wherein said immune checkpoint inhibitor that antagonizes PD-1 or PD-L1 is selected from the group consisting of avelumab, atezolizumab, balstilimab, budigalimab, camrelizumab, cosibelimab, dostarlimab, durvalumab, emiplimab, envafolimab ezabenlimab, nivolumab, pembrolizumab, pidilizumab, pimivalimab, retifanlimab, sasanlimab, spartalizumab, sintilimab, tislelizumab, toripalimab, and zimberelimab.

Embodiment 12: The method of embodiment 11, wherein said immune checkpoint inhibitor is zimberelimab.

Embodiment 13: The method of any one of embodiments 8-12, wherein said one or more immune checkpoint inhibitors comprise an immune checkpoint inhibitor that antagonizes TIGIT.

Embodiment 14: The method of embodiment 13, wherein said immune checkpoint inhibitor that antagonizes TIGIT is selected from the group consisting of AB308, domvanalimab, etigilimab, ociperlimab, tiragolumab, and vibostolimab.

Embodiment 15: The method of embodiment 14, wherein said immune checkpoint inhibitor is domvanalimab or AB308.

Embodiment 16: The method of any one of embodiments 7-15, wherein said at least one additional therapeutic agent comprises one or more agents that target the extracellular production of adenosine selected from the group consisting of an A2aR/A2bR antagonist, a CD73 inhibitor, and a CD39 inhibitor.

Embodiment 17: The method of embodiment 16, wherein the one or more agents that target the extracellular production of adenosine are selected from the group consisting of AB598, etrumadenant, inupadenant, taminadenant, caffeine citrate, imaradenant, ciforadenant, and quemliclustat.

Embodiment 18: The method of embodiment 17, wherein the one or more agents that target the extracellular production of adenosine are AB598, etrumadenant and/or quemliclustat.

Embodiment 19: The method of any one of embodiments 7-18, wherein said at least one additional therapeutic agent comprises an inhibitor of HIF-2α selected from the group consisting of belzutifan, ARO-HIF2, PT-2385, and AB521.

Embodiment 20: The method of embodiment 19, wherein said inhibitor of HIF-2a is AB521.

Embodiment 21: The method of any one of embodiments 7-20, wherein said at least one additional therapeutic agent comprises a chemotherapeutic agent.

Embodiment 22: The method of embodiment 21, wherein said chemotherapeutic agent comprises a purine-based, platinum-based, taxoid-based, or anthracycline-based chemotherapeutic agent.

Embodiment 23: The method of embodiment 22, wherein the chemotherapeutic agent is selected from the group consisting of cyclophosphamide, fludarabine, cisplatin, carboplatin, oxaliplatin, doxorubicin, docetaxel, and paclitaxel.

Embodiment 24: The method of any one of embodiments 7-23, wherein the at least one additional therapeutic agent comprises radiation therapy.

Embodiment 25: A combination comprising a compound according to this disclosure, or a pharmaceutically acceptable salt thereof, and at least one additional therapeutic agent.

Embodiment 26: The combination of embodiment 25, wherein said at least one additional therapeutic agent comprises one or more agents independently selected from the groups consisting of immune checkpoint inhibitors, agents that target the extracellular production of adenosine, inhibitors of HIF (e.g., a HIF-2α inhibitor), kinase inhibitors, radiation therapy, and chemotherapeutic agents.

Embodiment 27: The combination of embodiment 25 or 26, wherein said at least one additional therapeutic agent comprises one or more immune checkpoint inhibitors that antagonizes at least one of PD-1, PD-L1, BTLA, LAG-3, a B7 family member, TIM-3, TIGIT, or CTLA-4.

Embodiment 28: The combination of embodiment 27, wherein said one or more immune checkpoint inhibitors comprise an immune checkpoint inhibitor that antagonizes PD-1 or PD-L1.

Embodiment 29: The combination of embodiment 28, wherein said immune checkpoint inhibitor that antagonizes PD-1 or PD-L1 is selected from the group consisting of avelumab, atezolizumab, balstilimab, budigalimab, camrelizumab, cosibelimab, dostarlimab, durvalumab, emiplimab, envafolimab ezabenlimab, nivolumab, pembrolizumab, pidilizumab, pimivalimab, retifanlimab, sasanlimab, spartalizumab, sintilmab, tislelizumab, toripalimab, and zimberelimab.

Embodiment 30: The combination of embodiment 29, wherein said immune checkpoint inhibitor is zimberelimab.

Embodiment 31: The combination of any one of embodiments 26-30, wherein said one or more immune checkpoint inhibitors comprise an immune checkpoint inhibitor that antagonizes TIGIT.

Embodiment 32: The combination of embodiment 31, wherein said immune checkpoint inhibitor that antagonizes TIGIT is selected from the group consisting of AB308, domvanalimab, etigilimab, ociperlimab, tiragolumab, and vibostolimab.

Embodiment 33: The combination of embodiment 32, wherein said immune checkpoint inhibitor is domvanalimab or AB308.

Embodiment 34: The combination of any one of embodiments 25-33, wherein said at least one additional therapeutic agent comprises one or more agents that target the extracellular production of adenosine selected from the group consisting of an A2aR/A2bR antagonist, a CD73 inhibitor, and a CD39 inhibitor.

Embodiment 35: The combination of embodiment 34, wherein the one or more agents that target the extracellular production of adenosine are selected from the group consisting of AB598, etrumadenant, inupadenant, taminadenant, caffeine citrate, imaradenant, ciforadenant, and quemliclustat.

Embodiment 36: The combination of embodiment 35, wherein the one or more agents that target the extracellular production of adenosine are AB598, etrumadenant and/or quemliclustat.

Embodiment 37: The combination of any one of embodiments 25-36, wherein said at least one additional therapeutic agent comprises an inhibitor of HIF-2a selected from the group consisting of belzutifan, ARO-HIF2, PT-2385, and AB521.

Embodiment 38: The combination of embodiment 37, wherein said inhibitor of HIF-2a is AB521.

Embodiment 39: The combination of any one of embodiments 25-38, wherein said at least one additional therapeutic agent comprises a chemotherapeutic agent.

Embodiment 40: The combination of embodiment 39, wherein said chemotherapeutic agent comprises a platinum-based, taxoid-based, or anthracycline-based chemotherapeutic agent.

Embodiment 41: The combination of embodiment 40, wherein the chemotherapeutic agent is selected from the group consisting of cisplatin, carboplatin, oxaliplatin, doxorubicin, docetaxel, and paclitaxel.

Embodiment 42: The combination of any one of embodiments 25-41, wherein the at least one additional therapeutic agent comprises radiation therapy.

### EXPERIMENTAL

The following examples are put forth so as to provide those of ordinary skill in the art with a complete disclosure and description of how to make and use the present disclosure, and are not intended to limit the scope of what the inventors regard as their invention. Efforts have been made to ensure accuracy with respect to numbers used (e.g., amounts, temperature, etc.), but some experimental errors and deviations should be accounted for.

All reactions were performed using a Teflon-coated magnetic stir bar at the indicated temperature and were conducted under an inert atmosphere when stated. Purchased starting materials and reagents were generally used as received. Reactions were monitored by TLC (silica gel 60 with fluorescence F254, visualized with a short wave/long wave UV lamp) and/or LCMS (AGILENT® 1100 or 1200 series LCMS with UV detection at 254 or 280 nm using a binary solvent system [0.1% formic acid in MeCN/0.1% formic acid in H₂O] using one of the following columns: AGILENT® Eclipse Plus C18 [3.5 μm, 4.6 mm i.d.×100 mm], WATERS™ XSelect HSS C18 [3.5 μm, 2.1 mm i.d.×75 mm]). Flash chromatography was conducted on silica gel using an automated system (COMBIFLASH® RF+ manufactured by Teledyne ISCO), with detection wavelengths of 254 and 280 nm, and optionally equipped with an evaporative light scattering detector. Reverse phase preparative HPLC was conducted on an AGILENT® 1260 or 1290 Infinity series HPLC. Samples were eluted using a binary solvent system (MeCN/H₂O with an acid modifier as needed—for example 0.1% TFA or 0.1% formic acid) with gradient elution on a Gemini C18 110 Å column (21.2 mm i.d.××250 mm) with variable wavelength detection. Final compounds obtained through preparative HPLC were concentrated through lyophilization. All assayed compounds were purified to ≥95% purity as determined by ¹H NMR or LCMS (AGILENT® 1100 or 1200 series LCMS with UV detection at 254 or 280 nm using a binary solvent system [0.1% formic acid in MeCN/0.1% formic acid in H₂O] using one of the following columns: AGILENT® Eclipse Plus C18 [3.5 μm, 4.6 mm i.d.×100 mm], WATERS™ XSelect HSS C18 [3.5 μm, 2.1 mm i.d.×75 mm]). ¹H NMR spectra were recorded on a Varian 400 MHz NMR spectrometer equipped with an Oxford AS400 magnet or a BRUKER® AVANCE NEO 400 MHz NMR. Chemical shifts (6) are reported as parts per million (ppm) relative to residual undeuterated solvent as an internal reference. The abbreviations s, br s, d, t, q, dd, dt, ddd, and m stand for singlet, broad singlet, doublet, triplet, quartet, doublet of doublets, doublet of triplets, doublet of doublet of doublets, and multiplet, respectively.

Unless indicated otherwise, temperature is in degrees Celsius (° C.), and pressure is at or near atmospheric. Standard abbreviations are used, including the following: rt=room temperature; min(s)=minute(s); h=hour(s); mg=milligram; g=gram; kg=kilogram; μL=microliter; ml or mL=milliliter; M=molar; mol=mole; mmol=millimole; N=normality; sat.=saturated; aq.=aqueous; psi=pounds per square inch; calcd=calculated; equiv.=equivalents; NMP=N-methyl-2-pyrrolidone; PhMe=toluene; DCM and CH₂CL₂=dichloromethane; THF=tetrahydrofuran; EtOAc=ethyl acetate; TFA=trifluoroacetic acid; MeCN or CH₃CN=acetonitrile; DMF=N,N-dimethylformamide; DMSO=dimethyl sulfoxide; DMA=dimethylacetamide; DMF·DMA=N,N-dimethylformamide dimethyl acetal; EDC·HCl=N-(3-dimethylaminopropyl)-N′-ethylcarbodiimide hydrochloride; NBS=N-bromosuccinimide; CHCl₃=chloroform; CF₂HI=iododifluoromethane; MeOH=methanol; MTBE=methyl tert-butyl ether; N₂=nitrogen gas; H₂O₂=hydrogen peroxide; Boc₂O=di-tert-butyl decarbonate; T₃P=1-propanephosphonic anhydride solution; DIPEA and i-Pr₂NEt=N,N-diisopropylethylamine; DMAP=4-dimethylaminopyridine; DMEDA=N,N-dimethylethane-1,2-diamine; MeI=iodomethane; Et₃N=triethylamine; NaBH(OAc)₃=sodium triacetoxyborohydride; NH₄Cl=ammonium chloride; NH₃=ammonia; LiBF₄=lithium tetrafluoroborate; NaIO₄=sodium periodate; Na₂SO₄=sodium sulfate; NaNO₂=sodium nitrite; Na₂S₂O₃=sodium thiosulfate; NaHCO₃=sodium bicarbonate; NaH=sodium hydride; NaOH=sodium hydroxide; KI=potassium iodide; K₂CO₃=potassium carbonate; K₃PO₄=potassium phosphate; K₂OsO₄*2H₂O=potassium osmate(VI) dihydrate; LiI=lithium iodide; LiOH·H₂O=lithium hydroxide monohydrate; CuBr=copper bromide; CuI=copper iodide; Ag₂CO₃=silver carbonate; CuBr₂=copper(II) bromide; Ti(OiPr)₄=titanium(IV) isopropoxide; HCl=hydrochloric acid; TMSN₃=azidotrimethylsilane; HATU=1-[bis(dimethylamino)methylene]-1H-1,2,3-triazolo[4,5-b]pyridinium 3-oxide hexafluorophosphate; PhNTf₂=N-phenyl-bis(trifluoromethanesulfonimide); Deoxo-Fluor®=bis(2-methoxyethyl)aminosulfur trifluoride; L-Selectride®=lithium tri-sec-butylborohydride; XantPhos=(9,9-dimethyl-9H-xanthene-4,5-diyl)bis(diphenylphosphane); dppf=1,1′-bis(diphenylphosphino)ferrocene; Pd(dppf)Cl₂-[1,1′-bis(diphenylphosphino)ferrocene]palladium(II) dichloride; Xphos Pd G3=(2-dicyclohexylphosphino-2′,4′,6′-triisopropyl-1,1′-biphenyl)[2-(2′-amino-1,1′-biphenyl)]palladium(II) methanesulfonate; Xphos=dicyclohexyl[2′,4′,6′-tris(propan-2-yl)[1,1′-biphenyl]-2-yl]phosphane; Pd(dtBPF)Cl₂=[1,1′-bis(di-tert-butylphosphino)ferrocene]dichloropalladium(II); Pd(OAc)₂=palladium(II) acetate; Pd(PPh₃)₄=tetrakis(triphenylphosphine)palladium(0); Cp*Ru(COD)Cl=chloro(pentamethylcyclopentadienyl)(cyclooctadiene)ruthenium(II); NiCl₂·6H₂O=Nickel(II) chloride hexahydrate; Zn(CN)₂=zinc cyanide; MHz=megahertz; Hz=hertz; ppm=parts per million; TLC=thin layer chromatography; ESI MS=electrospray ionization mass spectrometry; LCMS=liquid chromatography-mass spectrometry; NMR=nuclear magnetic resonance; HPLC=high pressure liquid chromatography.

### Example 1: N-{4-[4-cyano-2-(4-methyl-4H-1,2,4-triazol-3-yl)phenyl]-6-cyclopropyl-2-pyridyl}-5-{[(S)-3-methyl-1-piperidyl]methyl}-1-cyclopropyl-2-oxo-1,2-dihydronicotinamide

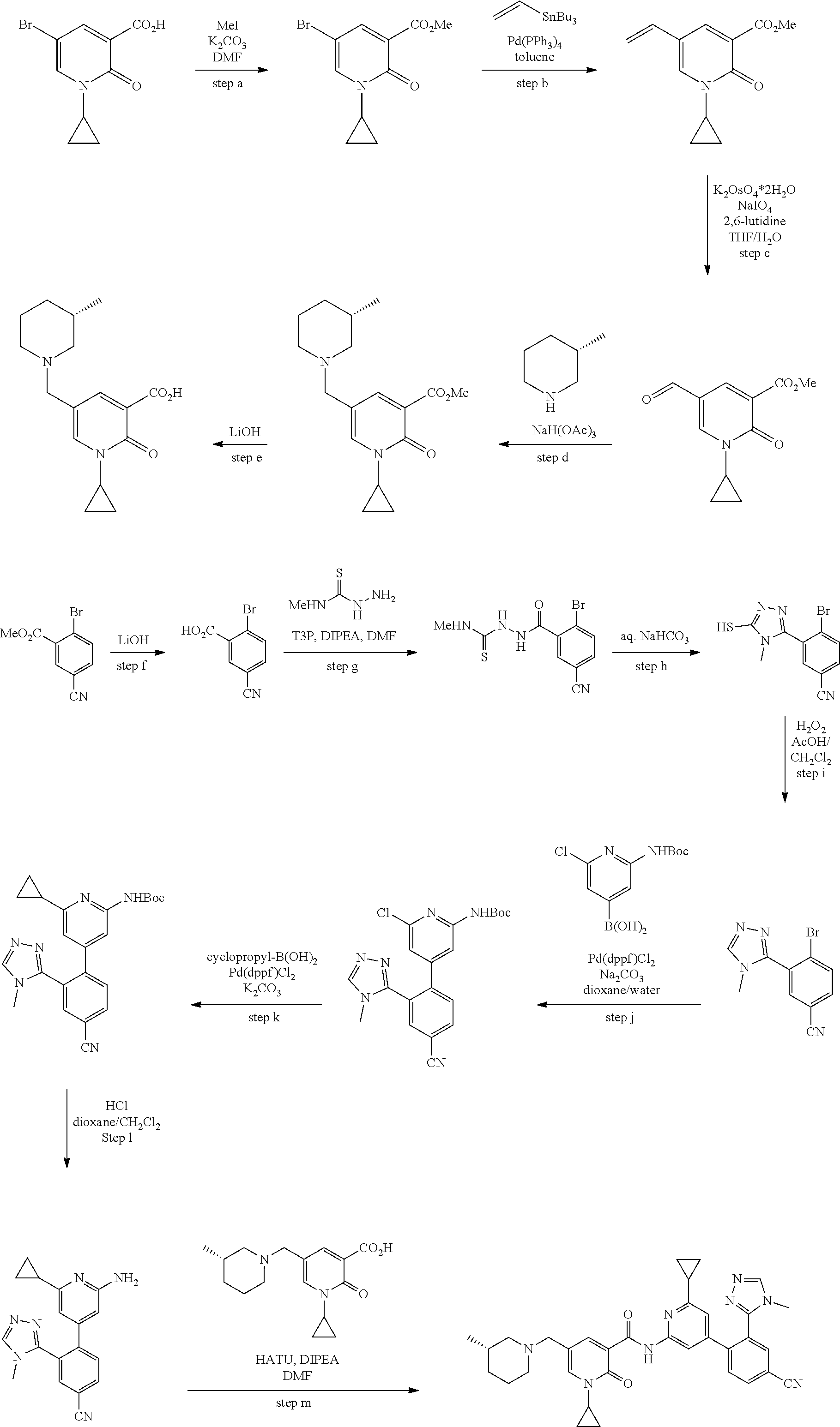

Step a: To a solution of 5-bromo-1-cyclopropyl-2-oxopyridine-3-carboxylic acid (2.55 g, 9.9 mmol, 1 equiv.) in DMF (50 mL, 0.2 M) anhydrous potassium carbonate (2.7 g, 19.8 mmol, 2 equiv.) was added. The mixture was cooled to 0° C., and MeI (0.7 mL, 10.9 mmol, 1.1 equiv.) was added in one portion. The resulting mixture was vigorously stirred at 0° C. for 10 min. Then the ice bath was removed, and the resulting suspension was stirred at room temperature for 4 h. Upon full consumption of starting material (TLC monitoring) the reaction mixture was poured in aq. sat. NH₄Cl (150 mL). The product was extracted with EtOAc (3×70 mL). The combined organic extract was washed with water (2×100 mL) and brine (150 mL), dried over Na₂SO₄ and concentrated to dryness under reduced pressure to produce crude methyl 5-bromo-1-cyclopropyl-2-oxopyridine-3-carboxylate. This material was used for the next step without further purification.

Step b: A mixture of the product from step a (2.05 g, 7.4 mmol, 1 equiv.), vinyl tributyltin (2.6 mL, 9.04 mmol, 1.2 equiv.), Pd(PPh₃)₄ (0.53 g, 0.75 mmol, 0.1 equiv.) and toluene (38 mL, 0.2 M) was loaded in a 100 mL round bottom flask equipped with a stirring bar, reflux condensed and nitrogen inlet. The resulting mixture was degassed under vacuum and backfilled with nitrogen (repeated 3 times), then refluxed for 3 h. Once TLC analysis indicated complete conversion of the starting material the heating bath was removed, and the reaction mixture was allowed to cool to room temperature. Then 1M aq. potassium fluoride solution was added (50 mL), and the resulting mixture was vigorously stirred at room temperature overnight. The formed precipitate was removed by filtration through a pad of CELITE®. The filtrate was transferred into separatory funnel, the organic layer was separated, and the aqueous phase was additionally extracted with EtOAc (2×40 mL). Combined organic solution was dried over Na₂SO₄ and concentrated to dryness under reduced pressure. The crude product was purified by column chromatography (SiO₂, 0-100% EtOAc gradient in hexanes) to produce methyl 1-cyclopropyl-5-ethenyl-2-oxopyridine-3-carboxylate as a yellow solid.

Step c: To a solution the product of step b (0.9 g, 3.6 mmol, 1 equiv.) in a mixture of THF (9 mL) and water (9 mL), 2,4-lutidine (0.85 mL, 7.2 mmol, 2 equiv.) was added. The resulting mixture was placed in cold water bath (˜15° C.), then sodium periodate (3.1 g, 14.4 mmol, 4 equiv.) and K₂OsO₄*2H₂O (70 mg, 0.18 mmol, 0.05 equiv.) were added sequentially. The resulting mixture was vigorously stirred for 4 h. During the reaction time a significant amount of white precipitate formed. Once TLC analysis confirmed complete reaction the mixture was diluted with water (30 mL) and EtOAc (40 mL). The aqueous phase was extracted with EtOAc (3×30 mL). Combined organic phase was washed with aq. 1M HCl (100 mL) and brine (100 mL), dried over Na₂SO₄ and concentrated to dryness to produce methyl 1-cyclopropyl-5-formyl-2-oxopyridine-3-carboxylate as a white powder.

Step d: To the product of step c (0.5 g, 2.3 mmol, 1.0 equiv.) in CH₂Cl₂ (23 mL, 0.1 M) was added (S)-3-methylpiperidine hydrochloride (0.31 g, 2.3 mmol, 1.0 equiv.) and DIPEA (0.8 mL, 4.6 mmol, 2.0 equiv.) and the mixture was stirred at 23° C. for 10 mins. Then NaBH(OAc)₃ (0.73 g, 3.5 mmol, 1.5 equiv.) was added, and the mixture was stirred for 12 h at ambient temperature. The reaction was quenched with aq. sat. NaHCO₃, the organic phase was separated, and the aqueous layer was additionally extracted with CH₂Cl₂ (2×20 mL). The combined organic phase was dried over Na₂SO₄, concentrated under reduced pressure, and the crude residue was purified by column chromatography (SiO₂, MeOH in CH₂Cl₂, 0 to 10%) to yield methyl 1-cyclopropyl-5-[[(3S)-3-methylpiperidin-1-yl]methyl]-2-oxopyridine-3-carboxylate.

Step e: To the product of step d (0.47 g, 1.54 mmol, 1.0 equiv.) in MeOH (7 mL) and water (1 mL) was added lithium hydroxide monohydrate (0.32 g, 7.7 mmol, 5 equiv.). The resulting mixture was stirred at ambient temperature for 3 h. The resulting mixture was carefully acidified with 12 M HCl to pH˜3 and concentrated to dryness. The residue was co-evaporated with dry MeOH (3×20 mL), then dissolved in dry MeOH, filtered through CELITE® pad to remove NaCl and concentrated to dryness under reduced pressure to produce 1-cyclopropyl-5-[[(3S)-3-methylpiperidin-1-yl]methyl]-2-oxopyridine-3-carboxylic acid that was used for the next step without purification.

Step f: To the solution of methyl 2-bromo-5-cyanobenzoate (1.0 g, 4.2 mmol, 1.0 equiv.) in a mixture of THF and H₂O (10 mL, 4:1 v/v, 0.4 M) was added lithium hydroxide monohydride (0.52 g, 12.5 mmol, 3.0 equiv.) at 23° C. The resulting mixture was stirred at this temperature for 3 h. Once complete reaction was observed (TLC monitoring) the mixture was acidified to pH˜3 with aq. HCl (4 M). The mixture was diluted with EtOAc (20 mL), and the organic phase was separated. The aqueous phase additionally extracted with EtOAc (2×10 mL), combined organic phase was washed with brine, dried over Na₂SO₄ and concentrated to dryness under reduced pressure to yield 2-bromo-5-cyanobenzoic acid.

Step g: To the product of step f (1.6 g, 7.1 mmol, 1.0 equiv.) in DMF (20 mL, 0.35 M) were sequentially added 1-amino-3-methylthiourea (0.9 g, 8.5 mmol, 1.2 equiv.), T₃P (10.6 g, 28.3 mmol, 4.0 equiv., 50 wt % solution in EtOAc) and DIPEA (8.8 mL, 42.48 mmol, 6.0 equiv.). The resulting mixture was stirred at 23° C. for 3 h. Then it was diluted with EtOAc (30 mL) and water (30 mL). The organic phase was separated, and the aqueous layer was additionally extracted with EtOAc (2×15 mL). The combined organic phase was dried over Na₂SO₄ and concentrated to dryness. The crude residue was purified by column chromatography (0-100% EtOAc gradient in hexanes) to yield 1-[(2-bromo-5-cyanobenzoyl)amino]-3-methylthiourea.

Step h: The product of step g (1.2 g, 3.8 mmol, 1.0 equiv.) was mixed with aq. NaHCO₃ (100 mL, 1.0 M), and the resulting suspension was stirred for 1 h at 80° C. Once the reaction was complete (LC/MS monitoring) it was allowed to cool to room temperature, the product was extracted with EtOAc (3×30 mL). Combined organic phase was dried over Na₂SO₄ and concentrated to dryness. The crude residue was purified by column chromatography (0-10% MeOH gradient in CH₂Cl₂) to yield 4-bromo-3-(4-methyl-5-sulfanyl-1,2,4-triazol-3-yl)benzonitrile.

Step i. To a solution of the product of step h (2.36 g, 8.0 mmol, 1.0 equiv.) in CH₂Cl₂ (20 mL, 0.4 M) at 0° C. was added AcOH (961 mg, 16.0 mmol, 2.0 equiv) followed by H₂O₂ (1.36 g, 40 mmol, 5 equiv., 30 wt %). The cooling bath was removed after 10 min, and the mixture was stirred at 23° C. for 2 h. The resulting mixture was diluted with H₂O, the organic phase was separated, and the aqueous phase was extracted with CH₂Cl₂ (2×20 mL). The combined organic phase was dried over Na₂SO₄, concentrated and the crude residue was purified by column chromatography (SiO₂, 0-20% MeOH in dichloromethane gradient) to afford 4-bromo-3-(4-methyl-1,2,4-triazol-3-yl)benzonitrile.

Step j: To a solution of the product of step i (0.17 g, 0.65 mmol, 1.0 equiv.) and [2-chloro-6-[(2-methylpropan-2-yl)oxycarbonylamino]pyridin-4-yl]boronic acid (0.18 g, 0.65 mmol, 1.0 equiv.) in dioxane (5.0 mL) Na₂CO₃ (0.21 g, 2.0 mmol, 3.0 equiv.) in water (1.0 mL) and Pd(dppf)Cl₂ (50 mg, 0.06 mmol, 0.1 equiv.) were added. The resulting mixture was degassed under vacuum and backfilled with nitrogen (repeated 3 times), then heated at 75° C. for 3 h. Once complete reaction was observed by LCMS analysis, the mixture was cooled to room temperature and diluted with sat. aq. NH₄Cl (10 mL) and EtOAc (20 mL). The organic phase was separated, and the aqueous layer was additionally extracted with EtOAc (2×10 mL). The combined organic phase was dried over Na₂SO₄ and concentrated to dryness under reduced pressure. The crude product was purified by column chromatography (SiO₂, 0-60% EtOAc gradient in hexanes) to produce tert-butyl N-[6-chloro-4-[4-cyano-2-(4-methyl-1,2,4-triazol-3-yl)phenyl]pyridin-2-yl]carbamate.

Step k: To a solution of the product of step j (155 mg, 0.38 mmol, 1.0 equiv.), cyclopropylboronic acid (65 mg, 0.76 mmol, 2.0 equiv.) and K₂CO₃ (160 mg, 1.14 mmol, 3.0 equiv.) in a dioxane/water mixture (6 mL, 5:1 v/v, 0.06 M), PdCl₂(dppf) (28 mg, 0.038 mmol, 0.1 equiv.) was added. The mixture was sparged with nitrogen for 3 min and heated at 90° C. for 12 h. The resulting mixture was cooled to room temperature and diluted with water (10 mL) and EtOAc (20 mL). The organic phase was separated, and the aqueous layer was extracted with EtOAc (2×10 mL). The combined organic phase was dried over Na₂SO₄, concentrated, and the crude tert-butyl N-[6-chloro-4-[4-cyano-2-(4-methyl-1,2,4-triazol-3-yl)phenyl]pyridin-2-yl]carbamate was used for the next step without purification.

Step 1: To a solution of the crude product from step j in CH₂Cl₂ (3 ml, 0.04 M) was added HCl solution in dioxane (1 ml, 4 M). The resulting solution was stirred for 1 h at 23° C. The mixture was concentrated to dryness, and the crude residue was used for the next step without purification.

Step m: To a solution of the crude product from step 1 (41 mg, 0.13 mmol, 1.0 equiv.) and the product of step e (43 mg, 0.13 mmol, 1.0 equiv.) in DMF (3 ml, 0.04 M) was add DIPEA (0.12 ml, 0.65 mmol, 5.0 equiv.). After 30 min of stirring at room temperature HATU (0.1 g, 0.26 mmol, 2.0 equiv.) was added to the above solution, and the mixture was stirred at room temperature overnight. The resulting mixture was directly fractionated by reversed-phase preparative HPLC (C18 column, 10-80% CH₃CN gradient in water with 0.1% formic acid) to furnish the title compound. ¹H NMR (400 MHz, CDCl₃) δ 12.31 (s, 1H), 8.51 (d, J=2.6 Hz, 1H), 8.20 (d, J=1.4 Hz, 1H), 8.06 (s, 1H), 7.95 (d, J=1.7 Hz, 1H), 7.91 (dd, J=8.1, 1.8 Hz, 1H), 7.77 (d, J=8.1 Hz, 1H), 7.63 (s, 1H), 6.49 (d, J=1.5 Hz, 1H), 3.46 (tt, J=7.6, 4.2 Hz, 1H), 3.34 (s, 2H), 3.09 (s, 3H), 2.79 (d, J=19.9 Hz, 2H), 1.93 (d, J=11.6 Hz, 1H), 1.81 (td, J=7.9, 4.0 Hz, 2H), 1.67 (q, J=26.1, 20.4 Hz, 4H), 1.25 (t, J=6.9 Hz, 2H), 0.97 (s, 2H), 0.93-0.81 (m, 8H). ESI MS [M+H]⁺ for C₃₄H₃₇N₈O₂, calcd 589.7, found 589.1.

### Example 2: 1-Cyclopropyl-N-[6-cyclopropyl-4-[4-fluoro-2-(4-methyl-1,2,4-triazol-3-yl)phenyl]pyridin-2-yl]-5-[[(3S)-3-methylpiperidin-1-yl]methyl]-2-oxopyridine-3-carboxamide

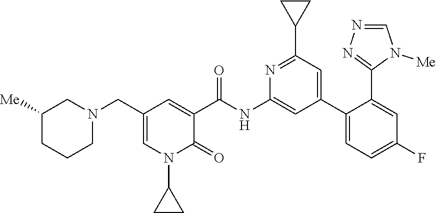 [SMILES: C[C@H]1CCCN(Cc2cc(C(=O)Nc3cc(-c4ccc(F)cc4-c4nncn4C)cc(C4CC4)n3)c(=O)n(C3CC3)c2)C1]

The title compound was prepared in a similar fashion to that described for Example 1 using 2-bromo-5-fluorobenzoic acid in step g. ¹H NMR (400 MHz, DMSO-d₆) δ 12.21 (s, 1H), 8.55-8.43 (m, 2H), 8.15 (d, J=2.6 Hz, 1H), 7.71-7.64 (m, 2H), 7.60-7.49 (m, 2H), 6.78 (d, J=1.5 Hz, 1H), 4.17-4.07 (m, 2H), 3.50-3.34 (m, 2H), 3.28 (s, 3H), 2.77-2.61 (m, 1H), 2.44-2.28 (m, 1H), 1.97 (td, J=8.3, 4.2 Hz, 1H), 1.85-1.48 (m, 4H), 1.14-1.05 (m, 2H), 1.05-0.98 (m, 1H), 0.98-0.89 (m, 5H), 0.86 (d, J=6.4 Hz, 3H), 0.81 (dt, J=4.6, 3.0 Hz, 2H). ESI MS [M+H]⁺ for C₃₃H₃₇FN₇O₂, calcd 582.3, found 582.3.

### Example 3: N-[4-[4-cyano-2-(4-methyl-1,2,4-triazol-3-yl)phenyl]-6-cyclopropylpyridin-2-yl]-1-cyclopropyl-5-[(2-methoxyethylamino)methyl]-2-oxopyridine-3-carboxamide

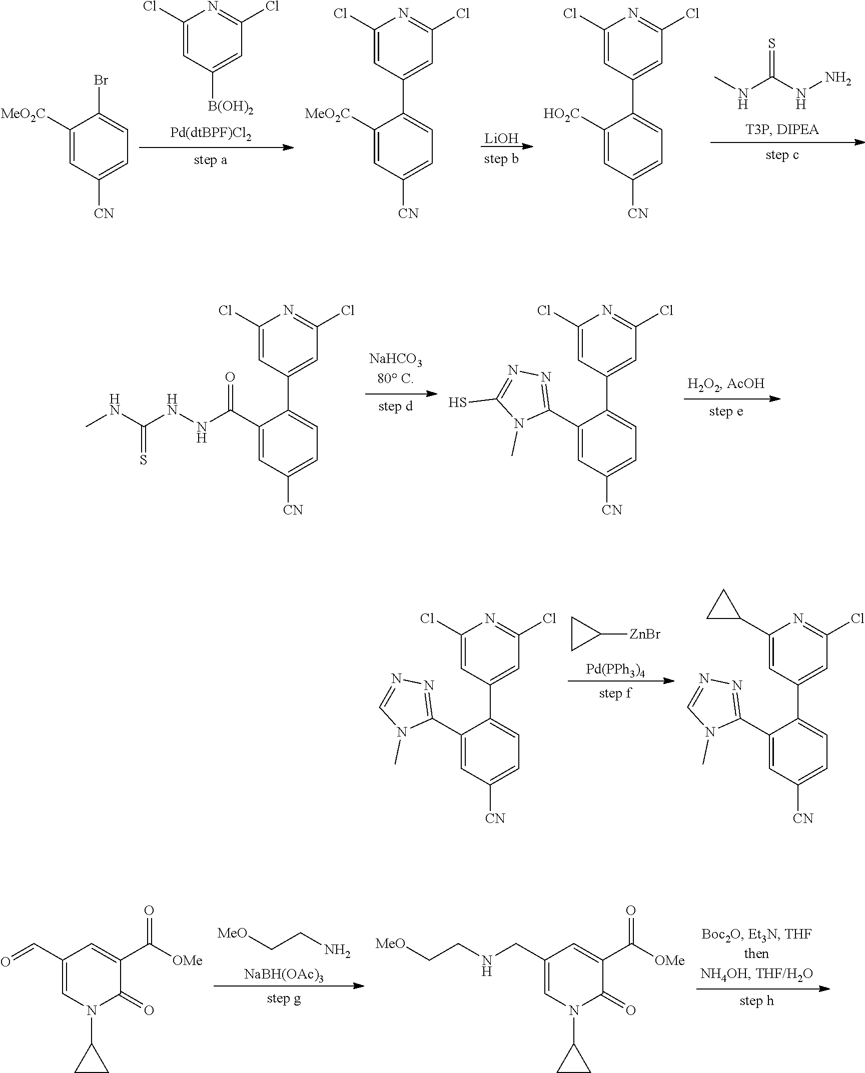

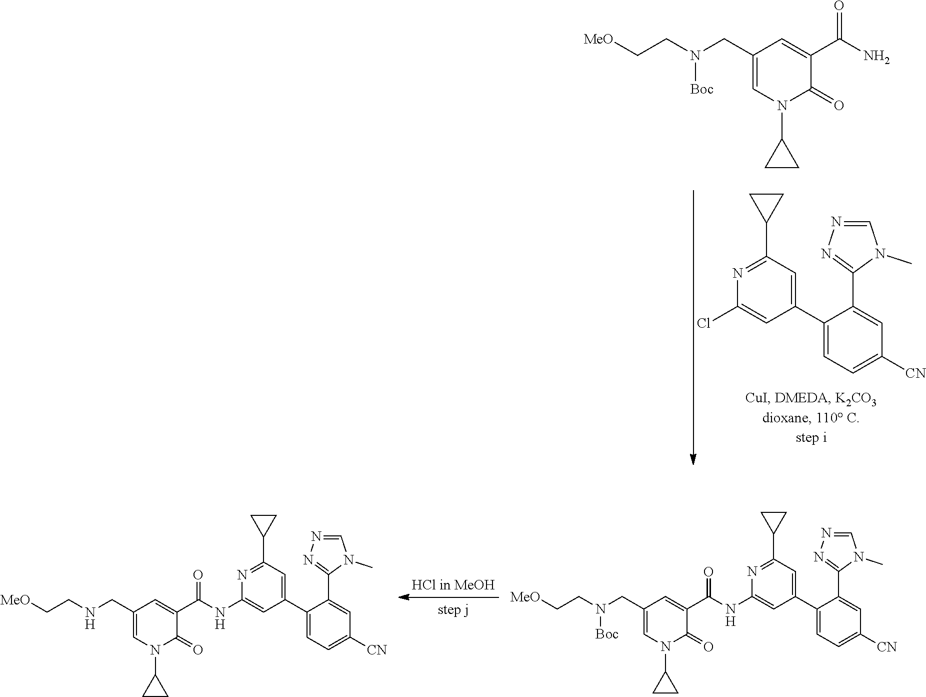

Step a: To a solution of methyl 2-bromo-5-cyanobenzoate (4.0 g, 16.7 mmol, 1.0 equiv.), (2,6-dichloropyridin-4-yl)boronic acid (3.84 g, 16.7 mmol, 1.0 equiv.) and K₂CO₃ (8.3 g, 50.0 mmol, 3.0 equiv.) in dioxane (40 mL) and H₂O (4 mL) was added Pd(dtBPF)Cl₂ (1.3 g, 1.666 mmol, 0.1 equiv.) at room temperature under nitrogen atmosphere. The resulting mixture was stirred for 3 h at 75° C. The resulting mixture was cooled to room temperature and quenched with sat. aq. NH₄Cl (20 mL). The resulting biphasic mixture was diluted with water (100 mL) and EtOAc (150 mL). The organic phase was separated, and the aqueous layer was extracted with EtOAc (2×50 mL). The combined organic phase was dried over Na₂SO₄, concentrated, and the crude residue was purified by column chromatography (SiO₂,0-60% EtOAc gradient in hexanes) to afford methyl 5-cyano-2-(2,6-dichloropyridin-4-yl)benzoate.

Step b: To the product of step a (1.45 g, 4.7 mmol, 1.0 equiv.) in THF/H₂O mixture (25 mL, 4:1 v/v, 0.2 M) was add LiOH (0.6 mg, 23.6 mmol, 5.0 equiv.). The reaction was stirred at room temperature for 3 h, and then acidified to pH˜3 with aq. HCl (4 M) at 0° C. Upon dilution with water (50 mL) and EtOAc (50 mL) the organic phase was separated, and the aqueous layer was extracted with EtOAc (30 mL). The combined organic phase was dried over Na₂SO₄ and concentrated under reduced pressure to give 5-cyano-2-(2,6-dichloropyridin-4-yl)benzoic acid that was directly used for the next step.

Step c: To the product of step b (1.32 g, 4.5 mmol, 1.0 equiv.) in DMF (20 mL, 0.2 M) was added 1-amino-3-methylthiourea (500 mg, 4.5 mmol, 1.0 equiv.), T₃P (6.5 g, 18.5 mmol, 4.0 equiv., 50% in EtOAc), and DIPEA (5.5 mL, 27 mmol, 6.0 equiv.). The mixture was stirred at 23° C. for 3 h. The reaction was diluted with H₂O (100 mL) and EtOAc (100 mL). The organic phase was separated, and the aqueous layer was additionally extracted with EtOAc (2×20 mL). The combined organic phase was dried over Na₂SO₄, concentrated and the crude residue was purified by column chromatography (SiO₂, 0-100% EtOAc in hexane) to yield 1-[[5-cyano-2-(2,6-dichloropyridin-4-yl)benzoyl]amino]-3-methylthiourea.

Step d: The product of step c (1.2 g, 3.158 mmol, 1.0 equiv.) was suspended in aq. sat. NaHCO₃ (100 mL, 1 M), then the reaction was stirred for 1 h at 80° C. Upon cooling it was diluted with EtOAc (150 mL), the organic phase was separated, and the aqueous layer was extracted with EtOAc (30 mL). The combined organic phase was dried over Na₂SO₄, concentrated, and the crude residue was purified by column chromatography (SiO₂, 0-20% MeOH gradient in dichloromethane) to produce 4-(2,6-dichloropyridin-4-yl)-3-(4-methyl-5-sulfanyl-1,2,4-triazol-3-yl)benzonitrile.

Step e: To the product of step d (0.75 g, 2.078 mmol, 1.0 equiv.) in dichloromethane (20 mL, 0.05 M) was added AcOH (0.26 g, 4.156 mmol, 2.0 equiv.). The reaction was cooled to 0° C. followed by the addition of H₂O₂ (1.2 g, 10.38 mmol, 15 equiv., 30 wt %). After 10 min the cooling bath was removed, and the mixture was stirred at 23° C. for 2 h. The reaction was diluted with H₂O, the organic phase was separated, and the aqueous layer was additionally extracted with dichloromethane (2×20 mL). The combined organic phase was dried over Na₂SO₄, concentrated and the crude residue was purified by column chromatography (SiO₂, 0-20% MeOH in dichloromethane) to afford 4-(2,6-dichloropyridin-4-yl)-3-(4-methyl-1,2,4-triazol-3-yl)benzonitrile.

Step f: To a solution of 4-(2,6-dichloropyridin-4-yl)-3-(4-methyl-1,2,4-triazol-3-yl)benzonitrile (0.11 g, 0.34 mmol, 1.0 equiv.) in THF (5 mL, 0.07M) under nitrogen atmosphere was added bromo(cyclopropyl)zinc (1.03 mL, 0.51 mmol, 1.5 equiv., 0.5 M) and Pd(PPh₃)₄ (80 mg, 0.07 mmol, 0.2 equiv.) at 23° C. under nitrogen atmosphere. The resulting mixture was stirred for 2 h at 65° C. in a sealed vial. Then it was cooled to room temperature and quenched with sat. aq. NH₄Cl (1 mL). The reaction was diluted with EtOAc (10 ml) and water (10 ml), the organic phase was separated, and the aqueous layer was extracted with EtOAc (2×10 mL). The combined organic phase was dried over Na₂SO₄, concentrated to dryness under reduced pressure. The crude residue was purified by column chromatography (SiO₂, 0-20% MeOH in dichloromethane) to 4-(2-chloro-6-cyclopropylpyridin-4-yl)-3-(4-methyl-1,2,4-triazol-3-yl)benzonitrile.

Step g: To the solution of methyl 1-cyclopropyl-5-formyl-2-oxopyridine-3-carboxylate (100 mg, 0.45 mmol, 1.0 equiv., prepared according to example 1) in dichloromethane (2.3 mL, 0.02 M) was added 2-methoxyethanamine (68 mg, 0.9 mmol, 2.0 equiv.) and DIPEA (0.2 mL, 1.12 mmol, 2.5 equiv.) and the mixture was stirred at 23° C. for 10 mins. NaBH(OAc)₃ (0.24 g, 1.12 mmol, 2.5 equiv.) was added and the mixture was stirred at 23° C. overnight. The reaction was quenched with aq. sat. NaHCO₃ (3 mL) and diluted with EtOAc (10 mL). The organic phase was separated, and the aqueous layer was additionally extracted with EtOAc (2×10 mL). The combined organic extract was dried over Na₂SO₄ and concentrated to dryness under reduced pressure. The crude residue was purified by column chromatography (SiO₂, 0-25% MeOH in dichlorormethane) to afford the desired amination product.

Step h: To the solution of the product from step g (1.8 g, 6.4 mmol, 1.0 equiv.) in THF (20 mL, 0.3 M) was added triethylamine (3.4 ml, 19.3 mmol, 3.0 equiv.) and Boc₂O (2.1 g, 9.7 mmol, 1.5 equiv.) and the mixture was stirred at 23° C. overnight. Then the mixture was concentrated to dryness under vacuum. The crude residue was dissolved in THF and water (90 mL, 2:1 v/v). Aq. conc. NH₄OH (4 ml) was added dropwise over 5 min, and the mixture was stirred overnight at 23° C. The product was extracted with EtOAc (2×50 mL), the combined organic phase was washed with water, dried over Na₂SO₄ and concentrated to dryness under reduced pressure. The crude residue was purified by column chromatography (SiO₂, 0-80% EtOAc gradient in hextane) to yield methyl 1-cyclopropyl-5-[[2-methoxyethyl-[(2-methylpropan-2-yl)oxycarbonyl]amino]methyl]-2-oxopyridine-3-carboxylate.

Step i: To a solution of the product from step h (80 mg, 0.21 mmol, 1.0 equiv.) and the product of step f (65 mg, 0.209 mmol, 1.0 equiv.) in dioxane (5 mL, 0.04 M) were added CuI (40 mg, 0.209 mmol, 1.0 equiv.), DMEDA (0.1 ml, 0.836 mmol, 4.0 equiv.) and K₂CO₃ (57 mg, 0.627 mmol, 3.0 equiv.). The resulting mixture was degassed under vacuum and backfilled with nitrogen (repeated 3 times). The resulting solution was stirred at 110° C. overnight. The mixture was cooled to room temperature, diluted with EtOAc (15 mL), aq. sat NH₄Cl (5 mL) and water (10 mL). The organic phase was separated, and the aqueous layer was extracted with EtOAc (10 mL). The combined organic phase was dried over Na₂SO₄, concentrated to dryness, and the crude residue was purified by column chromatography (SiO₂, 0-20% MeOH in dichlorormethane) to yield the desired product.

Step j: The product from step i was dissolved in a mixture of CH₂Cl₂ (3 ml) and HCl in MeOH (1 ml, 3M). The resulting solution was stirred at 23° C. for 1 h. The solvent was removed, and the crude product was purified by reversed phase preparative HPLC (C18, 10-90% CH₃CN in water, 0.1% formic acid) to furnish the title compound.¹H NMR (400 MHz, CDCl₃) δ 12.22 (s, 1H), 8.55 (d, J=2.7 Hz, 1H), 8.13 (d, J=1.5 Hz, 1H), 7.93 (d, J=1.7 Hz, 1H), 7.90-7.80 (m, 2H), 7.73 (d, J=8.1 Hz, 1H), 7.13 (d, J=1.3 Hz, 1H), 6.81 (d, J=1.3 Hz, 1H), 3.84 (s, 2H), 3.57 (dd, J=5.6, 4.4 Hz, 2H), 3.43 (tt, J=7.6, 4.2 Hz, 1H), 3.35 (s, 3H), 2.96 (dd, J=5.6, 4.4 Hz, 2H), 1.83 (tt, J=6.9, 5.9 Hz, 1H), 1.32-1.18 (m, 2H), 1.03-0.94 (m, 2H), 0.90-0.80 (m, 4H). ESI MS [M+H]⁺ for C₃₁H₃₃N₈O₃, calcd 565.3, found 565.1

### Example 4: 1-cyclopropyl-N-[6-cyclopropyl-4-[4-fluoro-2-(4-methyl-1,2,4-triazol-3-yl)phenyl]pyridin-2-yl]-5-[(2-methoxyethylamino)methyl]-2-oxopyridine-3-carboxamide

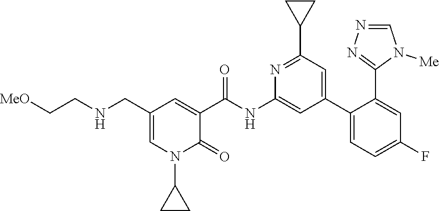 [SMILES: COCCNCc1cc(C(=O)Nc2cc(-c3ccc(F)cc3-c3nncn3C)cc(C3CC3)n2)c(=O)n(C2CC2)c1]

The title compound was prepared in a similar fashion to that described for Example 3 starting from methyl 2-bromo-5-fluorobenzoate in step a. ¹H NMR (400 MHz, DMSO-d₆) δ 12.38 (s, 1H), 8.46 (s, 1H), 8.40 (d, J=2.6 Hz, 1H), 7.89 (d, J=2.6 Hz, 1H), 7.71-7.65 (m, 2H), 7.55 (ddd, J=17.0, 8.6, 2.7 Hz, 2H), 6.70 (d, J=1.5 Hz, 1H), 3.56 (s, 2H), 3.44 (ddd, J=11.7, 7.7, 4.3 Hz, 1H), 3.36 (t, J=5.6 Hz, 3H), 3.31 (s, 3H), 3.21 (s, 3H), 2.62 (t, J=5.6 Hz, 2H), 1.94 (td, J=8.2, 4.2 Hz, 1H), 1.11-1.01 (m, 2H), 0.96-0.85 (m, 4H), 0.83-0.74 (m, 2H). ESI MS [M+H]+ for C30H33FN7O3, calcd 558.3, found 558.2.

### Example 5: 1-cyclopropyl-N-[6-ethoxy-4-[4-fluoro-2-(4-methyl-1,2,4-triazol-3-yl)phenyl]pyridin-2-yl]-5-[(2-methoxyethylamino)methyl]-2-oxopyridine-3-carboxamide

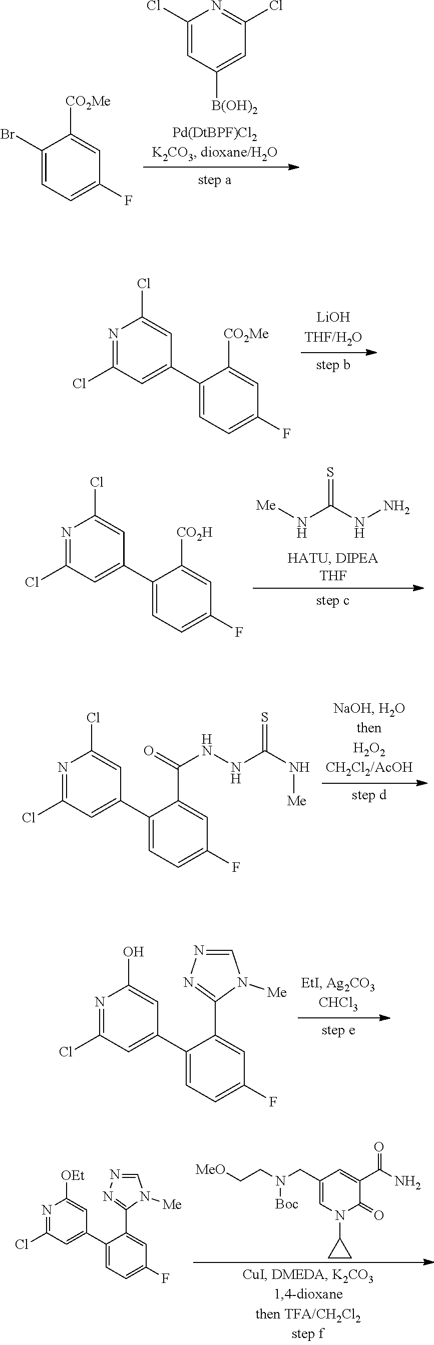

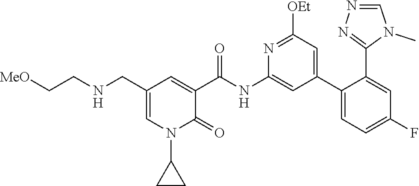 [SMILES: CCOc1cc(-c2ccc(F)cc2-c2nncn2C)cc(NC(=O)c2cc(CNCCOC)cn(C3CC3)c2=O)n1]

Step a: To a mixture of methyl 2-bromo-5-fluorobenzoate (4.66 g, 20 mmol, 1.0 equiv.) and (2,6-dichloropyridin-4-yl)boronic acid (4.60 g, 24 mmol, 1.2 equiv.) in dioxane/H₂O mixture (4:1 v/v, 100 mL) was added Pd(dtBPF)Cl₂ (1.30 g, 2.0 mmol, 0.1 equiv.) and K₂CO₃ (8.29 g, 60 mmol, 3.0 equiv.). After purging with nitrogen for 15 min the reaction was heated at 60° C. for 5 h. Then it was cooled to room temperature and diluted with EtOAc (80 mL) and water (60 mL). The organic phase was separated, and the aqueous layer was additionally extracted with EtOAc (2×30 mL). The combined organic phase was dried over Na₂SO₄, concentrated, and the crude residue was purified by column chromatography (SiO₂, 0-10% EtOAc gradient in hexanes) to afford biaryl coupling product.

Step b: To the solution of the product from step a (3.7 g, 12.5 mmol, 1.0 equiv.) in a THF/H₂O mixture (2:1 v/v, 60 mL) was added LiOH·H₂O (2.10 g, 50 mmol, 4.0 equiv.). The resulting mixture was heated at 60° C. for 2 h. Once complete conversion was observed (LC/MS analysis) the reaction was cooled to room temperature and acidified pH˜5 with 1 M HCl. The product was extracted with EtOAc (3×30 mL), combined organic phase was dried over Na₂SO₄ and concentrated to dryness under reduced pressure to afford corresponding carboxylic acid that used for the next step without purification.

Step c: To a solution of the product from step b (3.6 g, 12.4 mmol, 1.0 equiv.) and 1-amino-3-methylthiourea (1.3 g, 12.4 mmol, 1.0 equiv.) in THF (60 mL), HATU (4.71 g, 12.4 mmol, 1.0 equiv.) and DIPEA (6.5 mL, 37.2 mmol, 3.0 equiv.) were added. The resulting mixture was stirred at room temperature for 2 h. Then it was diluted with water (100 mL) and EtOAc (100 mL). The organic phase was separated, and the aqueous layer was additionally extracted with EtOAc (2×20 mL). The combined organic phase was dried over Na₂SO₄ and concentrated under reduced pressure. The crude product was directly used for the next step without purification.

Step d: To the crude product from step c (˜12.4 mmol) was added aq. 1 M NaOH (40 mL). The resulting homogenous mixture was heated at 100° C. for 1.5 h. Once the desired transformation completed (as judged by LCMS), the reaction mixture was cooled to room temperature, acidified with 1 M HCl to pH˜5, and diluted with EtOAc (60 mL). The organic phase was separated, and the aqueous layer was extracted with EtOAc (2×20 mL) twice. The combined organic phase was dried over Na₂SO₄, and the solvent was evaporated under reduced pressure. The crude residue was dissolved in dichloromethane (60 mL), followed by the addition of acetic acid (2.1 mL, 2.22 g, 37 mmol, 3.0 equiv.) and 30 wt % H₂O₂ aqueous solution (6.3 mL, 62 mmol, 5.0 equiv.) at 0° C. After 10 min the cooling bath was removed, and the resulting mixture was stirred at room temperature for 1 h. The resulting biphasic solution was quenched with aq. sat. NaHCO₃ to pH˜8. The resulting mixture was then extracted with dichloromethane/MeOH mixture (10:1 v/v, 4×25 mL). The combined organic phase was dried over Na₂SO₄, concentrated, and the crude product was purified by column chromatography (SiO₂, 0-10 MeOH gradient in dichloromethane) to afford 6-chloro-4-[4-fluoro-2-(4-methyl-1,2,4-triazol-3-yl)phenyl]pyridin-2-ol.

Step e: To a mixture of the product of step d (100 mg, 0.33 mmol, 1.0 equiv.) and Ag₂CO₃ (138 mg, 0.50 mmol, 1.5 equiv.) in CHCl₃ (3.0 mL) was added iodoethane (94.0 mg, 0.66 mmol, 2.0 equiv.). The resulting mixture was heated at 40° C. overnight before filtering through CELITE® and concentrating under vacuum. The crude residue was purified by column chromatography (SiO₂, 0-10% MeOH gradient in dichloromethane) to afford 2-chloro-6-ethoxy-4-[4-fluoro-2-(4-methyl-1,2,4-triazol-3-yl)phenyl]pyridine.

Step f: To a solution of the product from step e (54 mg, 0.16 mmol, 1.0 equiv.) and 1-cyclopropyl-5-[[2-methoxyethyl-[(2-methylpropan-2-yl)oxycarbonyl]amino]methyl]-2-oxopyridine-3-carboxylate (61 mg, 0.16 mmol, 1.0 equiv.) in dioxane (3 mL, 0.05 M) was added CuI (30 mg, 0.1621 mmol, 1.0 equiv.), DMEDA (0.1 ml, 0.6484 mmol, 4.0 equiv.) and K₂CO₃ (67 mg, 0.4863 mmol, 3.0 equiv.). The resulting solution was sparged with nitrogen for 10 min and stirred at 110° C. for 12 h. The mixture was cooled to room temperature, diluted with EtOAc (15 mL), aq. sat NH₄Cl (5 mL) and water (10 mL). The organic phase was separated, and the aqueous layer was extracted with EtOAc (5 mL). The combined organic phase was dried over Na₂SO₄, the solvent was removed, and the crude residue was purified by column chromatography (SiO₂, 0-20% OMeOH in dichloromethane) to give the crude product. The obtained Boc-protected compound was dissolved in a mixture of dichloromethane (3 ml) and TFA (1 ml). After stirring for 1 h the solution was concentrated to dryness, and the crude product was directly purified by reversed phase preparative HPLC (C18, 10-90% CH₃CN in water with 0.1% formic acid) to afford the title compound. ¹H NMR (400 MHz, CDCl₃) δ 12.16 (s, 1H), 8.53 (d, J=2.6 Hz, 1H), 8.07 (s, 1H), 7.83 (d, J=1.3 Hz, 1H), 7.74 (d, J=2.5 Hz, 1H), 7.62 (dd, J=8.5, 5.4 Hz, 1H), 7.39-7.29 (m, 2H), 6.19 (d, J=1.2 Hz, 1H), 4.26 (q, J=7.1 Hz, 2H), 3.74 (s, 2H), 3.54 (t, J=5.0 Hz, 2H), 3.44 (dt, J=7.3, 3.4 Hz, 1H), 3.36 (s, 3H), 3.19 (s, 3H), 2.87 (t, J=4.9 Hz, 2H), 1.32 (t, J=7.1 Hz, 3H), 1.24 (t, J=7.0 Hz, 2H), 0.97 (dd, J=6.4, 4.3 Hz, 2H). ESI MS [M+H]⁺ for C₂₉H₃₃FN₇O₄, calcd 562.3, found 562.1.

### Example 6: N-{6-cyclopropyl-4-[4-fluoro-2-(4-methyl-4H-1,2,4-triazol-3-yl)phenyl]-2-pyridyl}-1-cyclopropyl-5-{[(2-methoxyethyl)-N-methylamino]methyl}-2-oxo-1,2-dihydronicotinamide

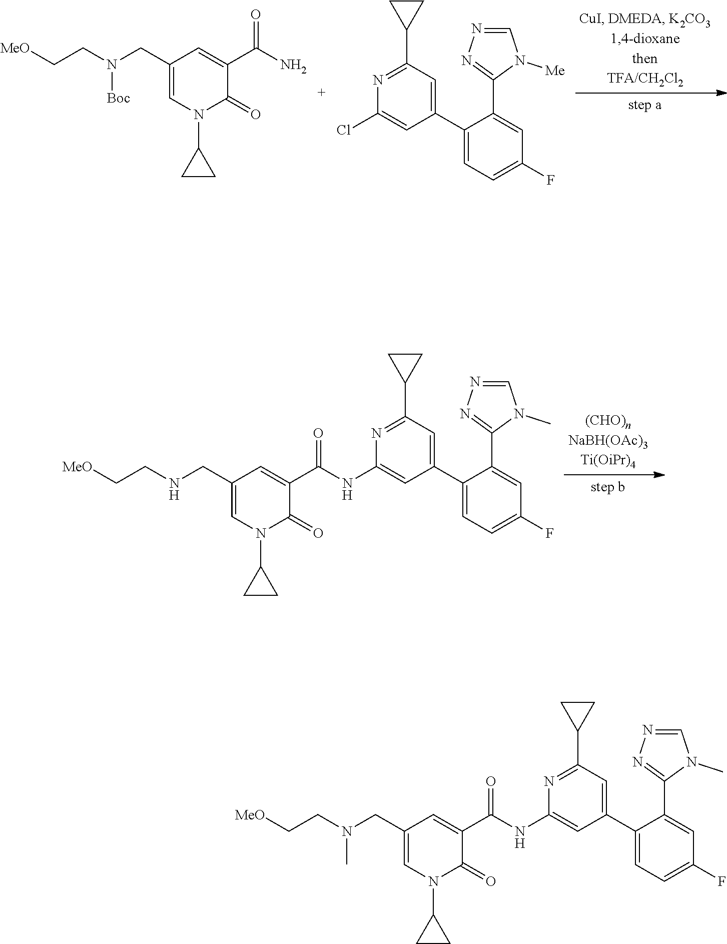 [SMILES: COCCN(Cc1cc(C(N)=O)c(=O)n(C2CC2)c1)C(=O)OC(C)(C)C ; Cn1cnnc1-c1cc(F)ccc1-c1cc(Cl)nc(C2CC2)c1 ; COCCNCc1cc(C(=O)Nc2cc(-c3ccc(F)cc3-c3nncn3C)cc(C3CC3)n2)c(=O)n(C2CC2)c1 ; COCCN(C)Cc1cc(C(=O)Nc2cc(-c3ccc(F)cc3-c3nncn3C)cc(C3CC3)n2)c(=O)n(C2CC2)c1]

Step a was performed in a similar fashion to step f of example 5.

Step b: To the product of step a (50 mg, 0.09 mmol, 1.0 equiv.) in THF (5 mL, 0.1 M) were added paraformaldehyde (6 mg, 0.18 mmol, 2.0 equiv.) and Ti(OiPr)₄ (0.1 mL, 0.4488 mmol, 5.0 equiv.). The reaction was stirred at 23° C. for 1 h, then NaBH(OAc)₃ (60 mg, 0.2693 mmol, 3.0 equiv.) was added, and the mixture was stirred overnight. The resulting solution was quenched with aq. sat. NaHCO₃ (10 mL) and diluted with EtOAc (20 mL). The organic layer was separated and the aqueous layer was extracted with EtOAc (10 mL). The combined organic phase was dried over Na₂SO₄, concentrated, and the crude residue was purified by column chromatography (SiO₂, 0-10% MeOH gradient in dichloromethane) to afford the title compound. ¹H NMR (400 MHz, CDCl₃) δ 12.24 (s, 1H), 8.48 (d, J=2.6 Hz, 1H), 8.15 (d, J=1.4 Hz, 1H), 8.04 (s, 1H), 7.74 (d, J=2.6 Hz, 1H), 7.63 (dd, J=8.6, 5.4 Hz, 1H), 7.41-7.30 (m, 2H), 6.46 (d, J=1.4 Hz, 1H), 3.58-3.41 (m, 5H), 3.37 (s, 3H), 3.09 (s, 3H), 2.66 (t, J=5.3 Hz, 2H), 2.34 (s, 3H), 1.81 (ddd, J=13.3, 8.0, 5.2 Hz, 1H), 1.25 (q, J=6.9 Hz, 2H), 0.99 (dd, J=6.5, 4.2 Hz, 2H), 0.88 (tt, J=8.0, 2.7 Hz, 4H). ESI MS [M+H]⁺ for C₃₁H₃₅FN₇O₃, calcd 572.3, found 572.1.

### Example 7: N-(4-{4-cyano-2-[4-(difluoromethyl)-4H-1,2,4-triazol-3-yl]phenyl}-6-cyclopropyl-2-pyridyl)-5-{[(S)-3-methyl-1-piperidyl]methyl}-1-cyclopropyl-2-oxo-1,2-dihydronicotinamide

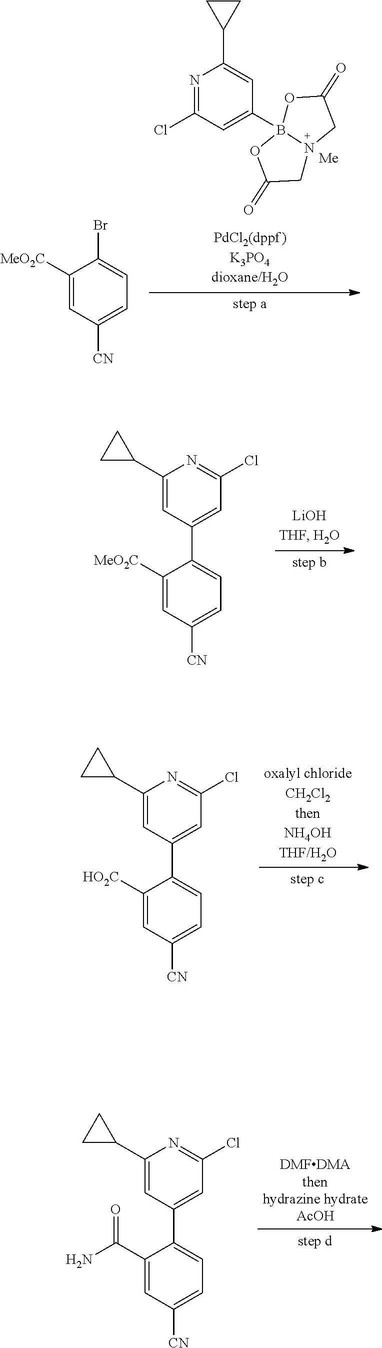

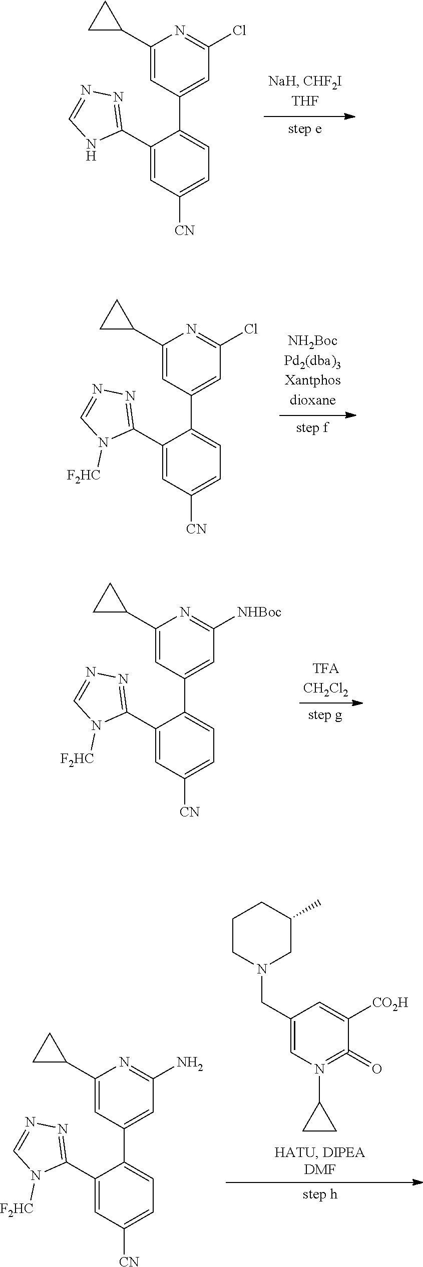

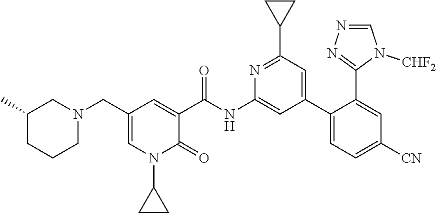 [SMILES: C[C@H]1CCCN(Cc2cc(C(=O)Nc3cc(-c4ccc(C#N)cc4-c4nncn4C(F)F)cc(C4CC4)n3)c(=O)n(C3CC3)c2)C1]

Step a: To a solution of methyl 2-bromo-5-cyanobenzoate (0.78 g, 3.25 mmol, 1.0 equiv.), 1-(2-chloro-6-cyclopropylpyridin-4-yl)-5-methyl-2,8-dioxa-5-azonia-1-boranuidabicyclo[3.3.0]octane-3,7-dione (1.0 g, 3.25 mmol, 1.0 equiv., prepared according to Example 15) and K₃PO₄ (2.1 g, 9.74 mmol, 3.0 equiv.) in dioxane (10 mL) and water (2 mL) mixture was added Pd(dppf)Cl₂ (0.74 g, 0.32 mmol, 0.1 equiv.). The mixture was degassed under vacuum and backfilled with nitrogen (repeated 2 times) and stirred at 90° C. for 1 h. Once near complete consumption of ester starting material was observed by TLC analysis the mixture was cooled to room temperature, diluted with EtOAc (100 mL) and washed with brine (100 ml). The organic phase was dried over Na₂SO₄, the solvent was removed under reduced pressure, and the material was purified by column chromatography (SiO₂, 0-60% EtOAc in hexane) to afford methyl 2-(2-chloro-6-cyclopropylpyridin-4-yl)-5-cyanobenzoate.

Step b: To the solution of the product of step a (1.0 g, 3.2 mmol, 1.0 equiv.) in a THF/H₂O mixture (24 mL, 5:1 v/v, 0.1 M) was added LiOH (0.38 g, 16.0 mmol, 5.0 equiv.). The reaction was stirred at 23° C. for 3 h. Once complete hydrolysis was observed by LC/MS analysis, the mixture was acidified to pH˜3 with aq. HCl (4 M) at 0° C. The reaction was diluted with EtOAc (50 mL), the organic phase was separated, and the aqueous layer was extracted with EtOAc (2×20 mL). The combined organic phase was dried over Na₂SO₄ and concentrated to dryness to afford 2-(2-chloro-6-cyclopropylpyridin-4-yl)-5-cyanobenzoic acid that was directly used for the next step.

Step c: To the solution of carboxylic acid from step b (1.7 g, 5.7 mmol, 1.0 equiv.) in CH₂Cl₂ (100 mL, 0.05 M) were added oxalyl chloride (1.1 ml, 11.4 mmol, 2.0 equiv.) and DMF (100 μL). The resulting mixture was stirred at 23° C. for 3 h. Then the mixture was concentrated to dryness under reduced pressure to afford crude acid chloride. It was dissolved in a THF and H₂O mixture (90 mL, 2:1 v/v) followed by addition of aq. conc. NH₄OH (4 ml). The mixture was stirred at room temperature overnight, then diluted with water (100 mL) and EtOAc (100 mL). The organic phase was separated, the aqueous phase was additionally extracted with EtOAc (2×30 mL). The combined organic extract was dried over Na₂SO₄ and concentrated to dryness under reduced pressure to afford 2-(2-chloro-6-cyclopropylpyridin-4-yl)-5-cyanobenzamide that was used for the next step without further purification.

Step d: The solution of the product of step c (120 mg, 0.36 mmol, 1.0 equiv.) in DMF·DMA (18 mL) was heated at 100° C. for 3 h. The mixture was allowed to cool to room temperature and the excess of DMF·DMA was removed under vacuum. The crude residue was dissolved in AcOH (15 ml), hydrazine hydrate (3.5 ml) was added dropwise over 5 min, and the resulting solution was stirred at room temperature for 2 h. The mixture was slowly basified with aq. sat. NaHCO₃ to pH˜5-6, and the product was extracted with EtOAc (3×25 mL). The combined organic extract was dried over Na₂SO₄ and concentrated to dryness under reduced pressure. The crude product was purified by column chromatography (SiO₂, 0-10% MeOH in dichloromethane) to afford 4-(2-chloro-6-cyclopropylpyridin-4-yl)-3-(4H-1,2,4-triazol-3-yl)benzonitrile.

Step e: To a solution of the product of step d (250 mg, 0.7788 mmol, 1.0 equiv.) in THF (5 mL, 0.15 M) was added NaH (62 mg, 1.5576 mmol, 2.0 equiv., 60% wt in oil) at 0° C. over 10 min. To the above solution was added CF₂HI (416 mg, 2.34 mmol, 3.0 equiv., 10% wt in acetonitrile). The mixture was stirred at rt for 3 h. The reaction was quenched with sat. aq. NH₄Cl solution, the organic phase was separated, and the aqueous layer was extracted with EtOAc. The combined organic phase was dried over Na₂SO₄, concentrated and the crude residue was purified by column chromatography (SiO2, MeOH in DCM, 0 to 10%) to 4-(2-chloro-6-cyclopropylpyridin-4-yl)-3-[4-(difluoromethyl)-1,2,4-triazol-3-yl]benzonitrile.

Step f: To the solution of the product of step e (45 mg, 0.121 mmol, 1.0 equiv.) in dioxane (3 mL, 0.04 M) was added tert-butyl carbamate (17 mg, 0.1452 mmol, 1.2 equiv.), Pd₂(dba)₃ (11 mg, 0.006 mmol, 5 mol %), XantPhos (14 mg, 0.012 mmol, 10 mol %). The resulting mixture was heated under N₂ at 90° C. for 1 h. After cooling to room temperature, the mixture was concentrated and the crude was purified by column chromatography (SiO₂, MeOH in DCM, 0 to 10%) to give tert-butyl N-[4-[4-cyano-2-[4-(difluoromethyl)-1,2,4-triazol-3-yl]phenyl]-6-cyclopropylpyridin-2-yl]carbamate.

Step g: To a solution of the crude product from step f (450 mg, 1.0 mmol, 1.0 equiv.) in DCM (5 ml, 0.2 M) was add TFA (3 ml, excess). The resulting solution was stirred at rt for 1 h. The solvent was removed, and the mixture was concentrated and the crude was purified by column chromatography (SiO₂, MeOH in DCM, 0 to 10%) to give 4-(2-amino-6-cyclopropylpyridin-4-yl)-3-[4-(difluoromethyl)-1,2,4-triazol-3-yl]benzonitrile.

Step h: To a solution of the product from step g (60 mg, 0.1699 mmol, 1.2 equiv.) and 1-cyclopropyl-5-[[(3S)-3-methylpiperidin-1-yl]methyl]-2-oxopyridine-3-carboxylic acid (see the synthetic details in Example 1) (42 mg, 0.1416 mmol, 1.0 equiv.) in DMF (3 ml, 0.05 M) was add DIPEA (0.1 ml, 0.4248 mmol, 3.0 equiv.). The resulting solution was stirred at rt for 0.5 h. Then, HATU (78 mg, 0.2124 mmol, 2.0 equiv.) was added to the above solution and the mixture was stirred for 12 h. The solvent was removed, and the mixture was concentrated, and the crude was purified by column chromatography (SiO2, MeOH in DCM, 0 to 10%) to give the title compound. ¹H NMR (400 MHz, CDCl₃) δ 12.32 (s, 1H), 8.50 (d, J=2.6 Hz, 1H), 8.45 (s, 1H), 8.12 (d, J=1.4 Hz, 1H), 7.97 (dd, J=9.5, 1.3 Hz, 2H), 7.80 (d, J=7.9 Hz, 1H), 7.66 (d, J=2.6 Hz, 1H), 6.73-6.31 (m, 2H), 3.46 (tt, J=7.6, 4.2 Hz, 1H), 3.37 (s, 2H), 2.81 (dd, J=22.5, 9.4 Hz, 2H), 1.95 (d, J=11.5 Hz, 1H), 1.83 (ddd, J=8.1, 5.6, 3.1 Hz, 1H), 1.76-1.48 (m, 6H), 1.26 (t, J=6.9 Hz, 2H), 1.05-0.77 (m, 9H). ESI MS [M+H]+ for C₃₄H₃₅F₂N₈O₂, calcd 600.3, found 600.1.

### Example 8: N-(6-amino-4-{4-cyano-2-[4-(difluoromethyl)-4H-1,2,4-triazol-3-yl]phenyl}-2-pyridyl)-5-{[(S)-3-methyl-1-piperidyl]methyl}-1-cyclopropyl-2-oxo-1,2-dihydronicotinamide

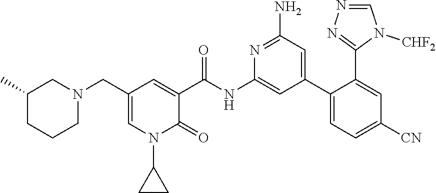 [SMILES: C[C@H]1CCCN(Cc2cc(C(=O)Nc3cc(-c4ccc(C#N)cc4-c4nncn4C(F)F)cc(N)n3)c(=O)n(C3CC3)c2)C1]

The title compound was prepared in a similar fashion to that described for Example 7 using (2,6-dichloropyridin-4-yl)boronic acid in step a. ¹H NMR (400 MHz, CDCl₃) δ 12.36 (s, 1H), 8.48 (s, 2H), 8.07-7.90 (m, 2H), 7.80 (d, J=8.0 Hz, 1H), 7.69 (d, J=7.1 Hz, 2H), 6.57 (t, J=59.5 Hz, 1H), 5.87 (s, 1H), 4.51 (s, 2H), 3.42 (d, J=13.7 Hz, 3H), 2.84 (dd, J=24.0, 9.0 Hz, 2H), 1.99 (s, 2H), 1.81-1.50 (m, 6H), 1.22 (d, J=7.3 Hz, 2H), 0.94 (d, J=5.0 Hz, 2H), 0.93-0.78 (m, 5H). ESI MS [M+H]⁺ for C₃₁H₃₂F₂N₉O₂, calcd 600.3, found 600.1.

### Example 9: N-(4-{4-cyano-2-[4-(difluoromethyl)-4H-1,2,4-triazol-3-yl]phenyl}-6-cyclopropyl-2-pyridyl)-1-cyclopropyl-2-oxo-5-[(2-trifluoromethoxyethylamino)methyl]-1,2-dihydronicotinamide

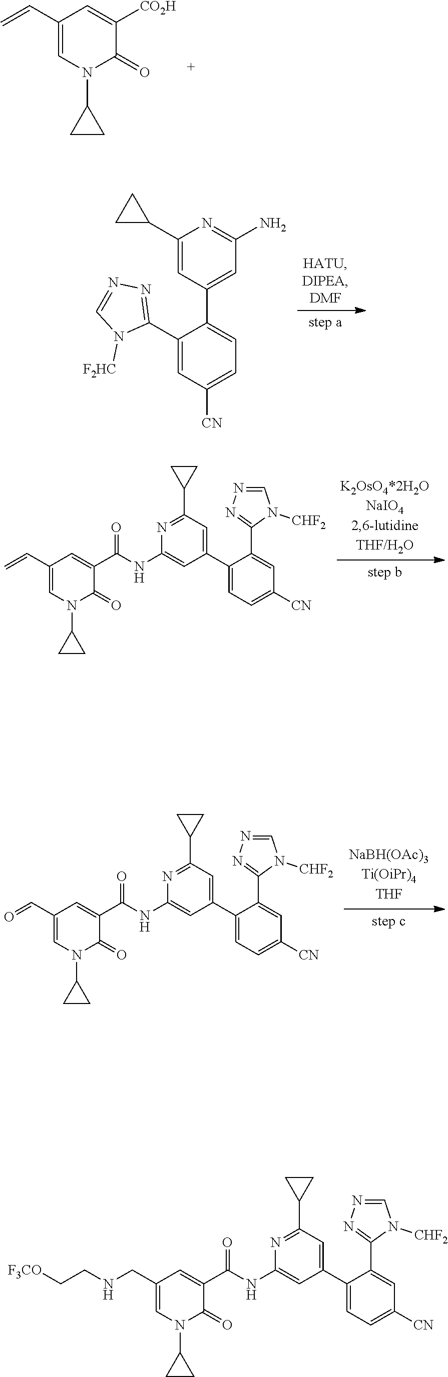 [SMILES: C=Cc1cc(C(=O)O)c(=O)n(C2CC2)c1 ; N#Cc1ccc(-c2cc(N)nc(C3CC3)c2)c(-c2nncn2C(F)F)c1 ; C=Cc1cc(C(=O)Nc2cc(-c3ccc(C#N)cc3-c3nncn3C(F)F)cc(C3CC3)n2)c(=O)n(C2CC2)c1 ; N#Cc1ccc(-c2cc(NC(=O)c3cc(C=O)cn(C4CC4)c3=O)nc(C3CC3)c2)c(-c2nncn2C(F)F)c1 ; N#Cc1ccc(-c2cc(NC(=O)c3cc(CNCCOC(F)(F)F)cn(C4CC4)c3=O)nc(C3CC3)c2)c(-c2nncn2C(F)F)c1]

Step a: To a solution of 1-cyclopropyl-5-ethenyl-2-oxopyridine-3-carboxylic acid (110 mg, 0.5376 mmol, 1.0 equiv.) (see synthetic details from Example 1) and 4-(2-amino-6-cyclopropylpyridin-4-yl)-3-[4-(difluoromethyl)-1,2,4-triazol-3-yl]benzonitrile (200 mg, 0.5376 mmol, 1.0 equiv.) (see synthetic details from Example 7) in DCM (5 ml, 0.1M) was add DIPEA (0.3 ml, 1.6128 mmol, 3.0 equiv.). The resulting solution was stirred at rt for 0.5 h. Then, HATU (306 mg, 0.8064 mmol, 1.5 equiv.) was added and the mixture was stirred for 12 h. The solvent was removed, and the mixture was concentrated and the crude material was purified by column chromatography (SiO₂, MeOH in DCM, 0 to 10%) to give N-[4-[4-cyano-2-[4-(difluoromethyl)-1,2,4-triazol-3-yl]phenyl]-6-cyclopropylpyridin-2-yl]-1-cyclopropyl-5-ethenyl-2-oxopyridine-3-carboxamide.

Step b: To a solution the product of step a (264 mg, 0.5029 mmol, 1.0 equiv.) in a mixture of THF (3 mL, 0.1 M) and water (3 mL, 0.1 M), 2,4-lutidine (110 mg, 1.0057 mmol, 2.0 equiv.) was added. The resulting mixture was placed in water bath, then sodium periodate (430 mg, 2.0114 mmol, 4.0 equiv.) and K₂OsO₄*2H₂O (10 mg, 0.02515 mmol, 0.05 equiv.) were added sequentially. The resulting mixture was vigorously stirred for 4 h. During the reaction time a significant amount of white precipitate formed. Once TLC analysis confirmed complete reaction the mixture was diluted with water (30 mL) and EtOAc (40 mL). The aqueous phase was extracted with EtOAc (3×30 mL). Combined organic phase was washed with brine (100 mL), dried over Na₂SO₄ and concentrated to dryness to produce N-[4-[4-cyano-2-[4-(difluoromethyl)-1,2,4-triazol-3-yl]phenyl]-6-cyclopropylpyridin-2-yl]-1-cyclopropyl-5-formyl-2-oxopyridine-3-carboxamide.

Step c: To the product of step b (60 mg, 0.12 mmol, 1.0 equiv.) in THF (5 mL, 0.1 M) were added 2-(trifluoromethoxy)ethanamine (37 mg, 0.24 mmol, 2.0 equiv.) and Ti(OiPr)₄ (0.16 mL, 0.6 mmol, 5.0 equiv.) and the mixture was stirred at 23° C. for 1 h. NaBH(OAc)₃ (62 mg, 0.3 mmol, 2.5 equiv.) was added, and the reaction was stirred overnight. The obtained mixture was quenched with aq. sat. NaHCO₃ (5 mL) and diluted with EtOAc (20 ml). The organic phase was separated, and the aqueous layer was extracted with EtOAc (2×10 mL). The combined organic phase was dried over Na₂SO₄, concentrated and the crude residue was purified by column chromatography (SiO₂, 0-10% MeOH gradient in dichloromethane) to yield the title compound. ¹H NMR (400 MHz, CDCl₃) δ 12.29 (s, 1H), 8.53 (d, J=2.6 Hz, 1H), 8.46 (s, 1H), 8.08 (d, J=1.4 Hz, 1H), 7.97 (d, J=7.8 Hz, 2H), 7.83-7.73 (m, 1H), 7.66 (d, J=2.6 Hz, 1H), 6.80-6.28 (m, 2H), 4.10 (t, J=5.0 Hz, 2H), 3.70 (s, 2H), 3.45 (tt, J=7.6, 4.2 Hz, 1H), 2.95 (t, J=5.1 Hz, 2H), 1.84 (tt, J=8.0, 4.9 Hz, 1H), 1.26 (dd, J=7.6, 6.2 Hz, 2H), 1.12-0.72 (m, 6H). ESI MS [M+H]⁺ for C₃₁H₂₈F₅N₈O₃, calcd 655.3, found 655.1.

### Example 10: N-(4-{4-cyano-2-[4-(difluoromethyl)-4H-1,2,4-triazol-3-yl]phenyl}-6-cyclopropyl-2-pyridyl)-1-cyclopropyl-5-[(1-methylcyclobutylamino)methyl]-2-oxo-1,2-dihydronicotinamide

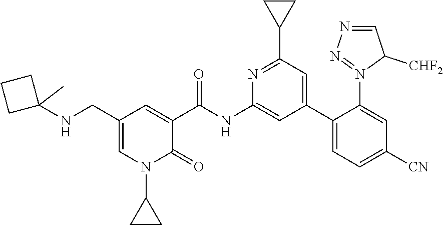

The title compound was prepared in a similar fashion to that described for Example 9) using 1-methylcyclobutan-1-amine hydrochloride in step c. ¹H NMR (400 MHz, CDCl₃) δ 12.29 (s, 1H), 8.52 (d, J=2.6 Hz, 1H), 8.49 (s, 1H), 8.07 (d, J=1.4 Hz, 1H), 8.00-7.93 (m, 2H), 7.83-7.78 (m, 1H), 7.71 (s, 1H), 6.88-6.27 (m, 2H), 3.58 (s, 2H), 3.43 (tt, J=7.6, 4.2 Hz, 1H), 2.02 (q, J=9.7, 9.0 Hz, 3H), 1.83 (m, 4H), 1.35 (s, 3H), 1.24 (q, J=6.9 Hz, 2H), 1.06-0.82 (m, 6H). ESI MS [M+H]⁺ for C₃₃H₃₃F₂N₈O₂, calcd 611.3, found 611.1.

### Example 11: N-(4-{4-cyano-2-[4-(difluoromethyl)-4H-1,2,4-triazol-3-yl]phenyl}-6-cyclopropyl-2-pyridyl)-5-({(1R,4S)-2-azabicyclo[2.2.1]hept-2-yl}methyl)-1-cyclopropyl-2-oxo-1,2-dihydronicotinamide

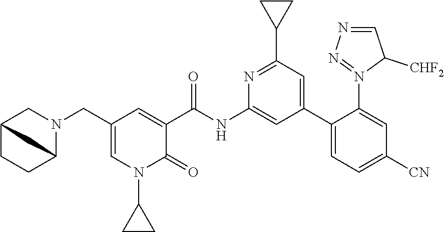

The title compound was prepared in a similar fashion to that described for Example 9 using (1R,4S)-2-aza-bicyclo[2.2.1]heptane hydrochloride in step c. ¹H NMR (400 MHz, CDCl₃) δ 12.21 (s, 1H), 8.58-8.27 (m, 2H), 8.21-8.01 (m, 2H), 8.01-7.90 (m, 2H), 7.79 (d, J=8.0 Hz, 1H), 6.73-6.30 (m, 2H), 3.92-3.73 (m, 2H), 3.58 (s, 1H), 3.48 (tt, J=7.7, 4.3 Hz, 1H), 2.90 (s, 1H), 2.71 (s, 1H), 2.59 (s, 1H), 2.01-1.46 (m, 6H), 1.26 (d, J=7.1 Hz, 2H), 1.12-0.84 (m, 6H). ESI MS [M+H]⁺ for C₃₄H₃₃F₂N₈O₂, calcd 623.3, found 623.1.

### Example 12: N-(4-{4-cyano-2-[4-(difluoromethyl)-4H-1,2,4-triazol-3-yl]phenyl}-6-cyclopropyl-2-pyridyl)-1-cyclopropyl-5-{[1-(methoxymethyl)cyclobutylamino]methyl}-2-oxo-1,2-dihydronicotinamide

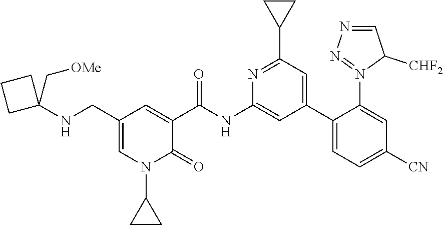

The title compound was prepared in a similar fashion to that described for Example 9 using 1-(methoxymethyl)cyclobutan-1-amine hydrochloride in step c. ¹H NMR (400 MHz, CDCl₃) δ 12.08 (s, 1H), 8.64 (s, 1H), 8.47 (s, 1H), 7.96 (d, J=13.1 Hz, 2H), 7.76 (d, J=9.7 Hz, 2H), 6.98-6.35 (m, 2H), 3.95 (s, 2H), 3.72 (s, 2H), 3.60 (m, 1H), 3.43 (s, 3H), 2.44 (s, 2H), 2.24 (q, J=11.2, 10.1 Hz, 1H), 2.03 (d, J=13.2 Hz, 3H), 1.93-1.73 (m, 2H), 1.18 (d, J=7.1 Hz, 2H), 0.97 (d, J=15.4 Hz, 6H). ESI MS [M+H]+ for C₃₄H₃₅F₂N₈O₃, calcd 641.3, found 641.1.

### Example 13: N-(4-{4-cyano-2-[4-(difluoromethyl)-4H-1,2,4-triazol-3-yl]phenyl}-6-cyclopropyl-2-pyridyl)-5-{[(S)-tetrahydro-3-furylamino]methyl}-1-cyclopropyl-2-oxo-1,2-dihydronicotinamide

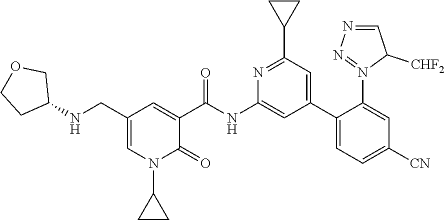

The title compound was prepared in a similar fashion to that described for Example 9 using (3R)-oxolan-3-amine in step c. ¹H NMR (400 MHz, CDCl₃) δ 12.29 (s, 1H), 8.52 (d, J=2.7 Hz, 1H), 8.45 (s, 1H), 8.08 (d, J=1.4 Hz, 1H), 7.97 (d, J=7.6 Hz, 2H), 7.81-7.75 (m, 1H), 7.65 (d, J=2.6 Hz, 1H), 6.90-6.09 (m, 2H), 4.12 (q, J=7.1 Hz, 2H), 4.01-3.91 (m, 1H), 3.86-3.75 (m, 2H), 3.71-3.59 (m, 2H), 3.49 (s, 2H), 3.44 (dq, J=8.1, 4.4, 4.0 Hz, 2H), 2.22-2.09 (m, 1H), 1.89-1.73 (m, 1H), 1.25 (d, J=2.7 Hz, 2H), 0.96 (dt, J=5.1, 2.7 Hz, 4H), 0.91 (dt, J=8.1, 2.8 Hz, 2H). ESI MS [M+H]+ for C₃₂H₃₁F₂N₈O₃, calcd 613.3, found 613.1.

### Example 14: N-(4-{4-cyano-2-[4-(difluoromethyl)-4H-1,2,4-triazol-3-yl]phenyl}-6-cyclopropyl-2-pyridyl)-1-cyclopropyl-5-[(2-methoxyethylamino)methyl]-2-oxo-1,2-dihydronicotinamide

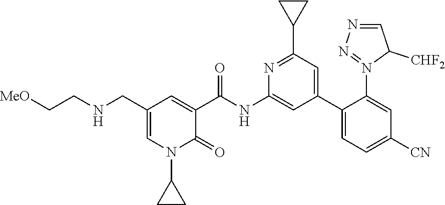

The title compound was prepared in a similar fashion to that described for Example 9 using 2-methoxyethanamine in step c. ¹H NMR (400 MHz, CDCl₃) δ 12.30 (s, 1H), 8.52 (d, J=2.6 Hz, 1H), 8.45 (s, 1H), 8.08 (d, J=1.5 Hz, 1H), 7.97 (d, J=7.5 Hz, 2H), 7.85-7.75 (m, 1H), 7.68 (d, J=2.6 Hz, 1H), 6.90-6.11 (m, 2H), 3.69 (s, 2H), 3.58-3.48 (m, 2H), 3.44 (dq, J=7.6, 3.8 Hz, 1H), 3.37 (s, 3H), 2.93-2.76 (m, 2H), 1.84 (ddd, J=13.2, 8.1, 4.9 Hz, 1H), 1.25 (q, J=6.9 Hz, 2H), 0.96 (dq, J=5.2, 2.7 Hz, 4H), 0.91 (dt, J=8.1, 2.9 Hz, 2H). ESI MS [M+H]⁺ for C₃₁H₃₁F₂N₈O₃, calcd 601.3, found 601.1.

### Example 15: N-{6-cyclopropyl-4-[4-fluoro-2-(4-methyl-4H-1,2,4-triazol-3-yl)phenyl]-2-pyridyl}-1-cyclopropyl-2-oxo-5-[(3,3,3-trifluoropropylamino)methyl]-1,2-dihydronicotinamide

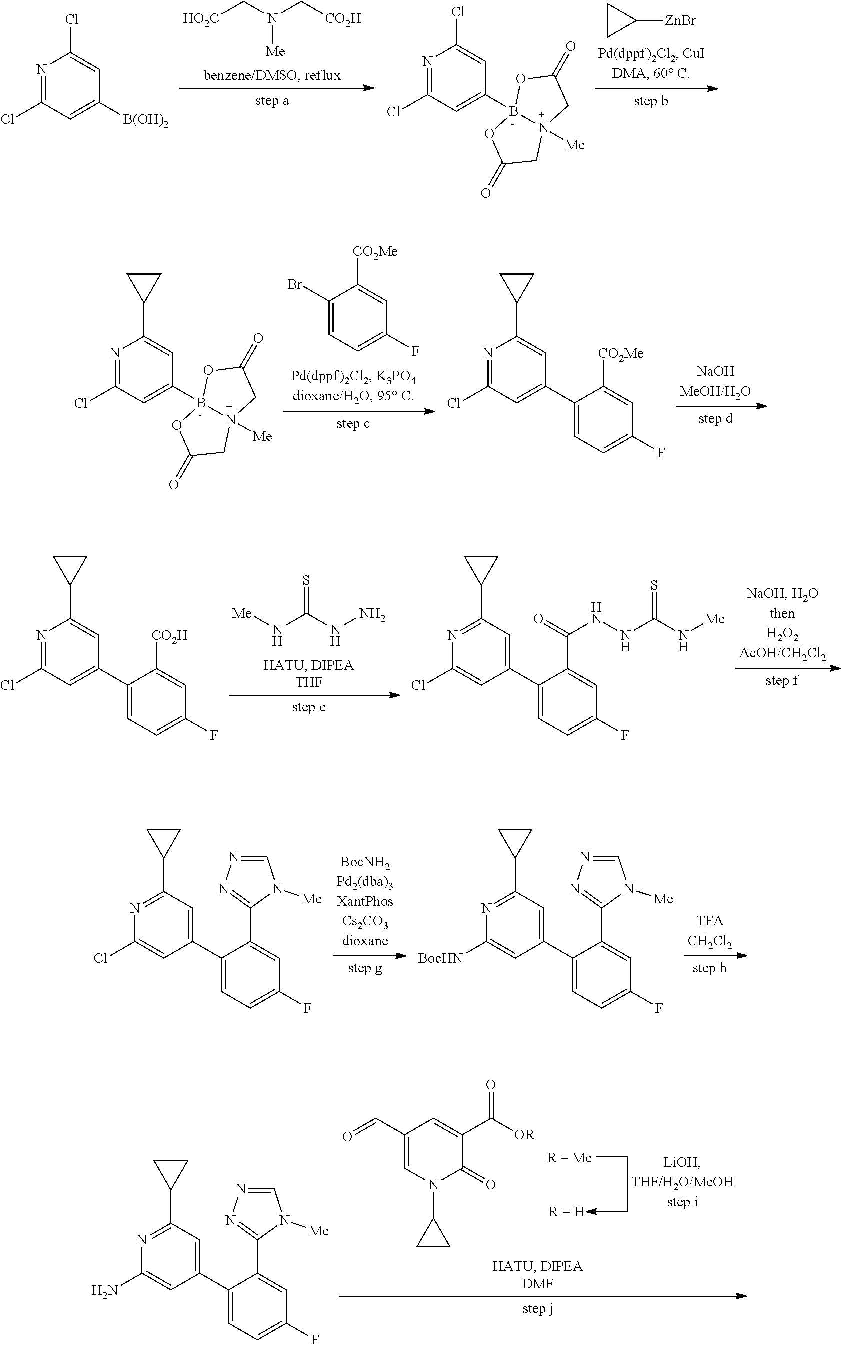

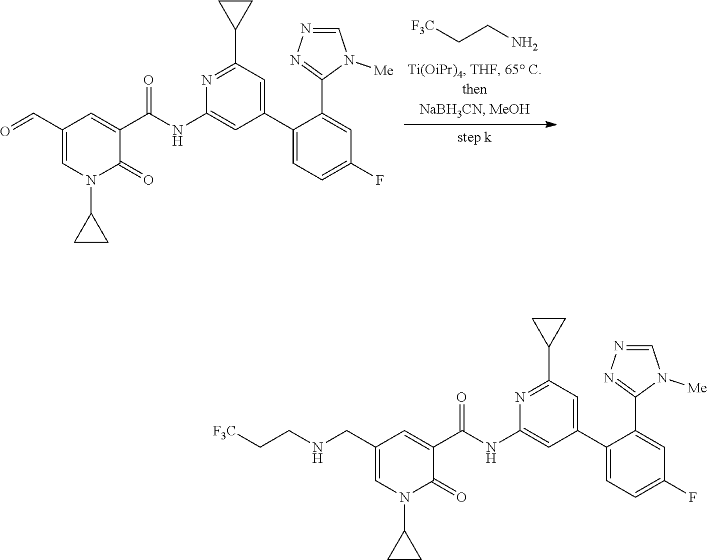 [SMILES: Cn1cnnc1-c1cc(F)ccc1-c1cc(NC(=O)c2cc(C=O)cn(C3CC3)c2=O)nc(C2CC2)c1 ; NCCC(F)(F)F ; Cn1cnnc1-c1cc(F)ccc1-c1cc(NC(=O)c2cc(CNCCC(F)(F)F)cn(C3CC3)c2=O)nc(C2CC2)c1]

Step a: To a 2 L round bottom flask was added (2,6-dichloropyridin-4-yl)boronic acid (13.8 g, 72.2 mmol, 1.0 equiv.) and 2-[carboxymethyl(methyl)amino]acetic acid (10.6 g, 72.2 mmol, 1.0 equiv.). The reagents were dissolved in a 9:1 mixture of benzene/DMSO (720 mL), a Dean-Stark apparatus was attached to the round bottom flask, and the reaction mixture was heated to a vigorous reflux for 16 hours (heating block temperature 130° C., ˜3 mL of water collected in Dean-Stark apparatus). The reaction mixture was cooled to room temperature and the benzene was removed under vacuum. The remaining DMSO solution was poured into 600 mL of water. The resulting solid was filtered, washed with water (2×300 mL), and dried on the filter frit to afford the desired product as a white solid.

Step b: A solution of the product from step a (21.6 g, 71.3 mmol, 1.0 equiv.) in DMA (50 mL) was sparged with N₂ for 15 minutes at which point CuI (552 mg, 2.9 mmol, 0.04 equiv.) and Pd(dppf)₂Cl₂ (1.54 g, 2.1 mmol, 0.03 equiv.) were added. Bromo(cyclopropyl)zinc (0.5 M in THF, 107 mmol, 214 mL, 1.5 equiv.) was added via cannula in a continuous stream and the reaction mixture was heated to 60° C. and stirred for 2 hours under N₂. The reaction mixture was cooled to room temperature and poured into saturated aqueous NH₄Cl (1 L) and extracted with EtOAc (2×350 mL). The combined organics were dried over Na₂SO₄, filtered, and concentrated under vacuum. The crude residue was triturated with CH₂C₂ to afford the desired product as a pale brown solid.

Step c: A 40 mL vial was charged with 1-(2-chloro-6-cyclopropylpyridin-4-yl)-5-methyl-2,8-dioxa-5-azonia-1-boranuidabicyclo[3.3.0]octane-3,7-dione (0.3 g, 1.0 mmol, 1 equiv.), methyl 2-bromo-5-fluorobenzoate (233 mg, 1 mmol. 1 equiv.) and K₃PO₄ (0.64 g, 3.0 mmol, 3 equiv.). The reagents were suspended in a 4:1 mixture of dioxane/water (10 mL) and the resulting solution was sparged with N₂ for 10 minutes. Then Pd(dppf)Cl₂ (73 mg, 0.1 mmol, 0.1 equiv.) was added, and the mixture was heated to 95° C. for 1 h. The reaction mixture was cooled to room temperature and partitioned between EtOAc (50 mL) and water (50 mL). The organic phase was separated, and the aqueous phase was additionally extracted with EtOAc (2×25 mL). The combined organic extract was dried over Na₂SO₄ and concentrated to dryness under reduced pressure. The crude residue was purified via column chromatography (SiO₂, 0-50% EtOAc gradient in hexanes) to afford methyl 2-(2-chloro-6-cyclopropylpyridin-4-yl)-5-fluorobenzoate as the product.

Step d: To a solution of the ester product from step c (2.36 g, 7.76 mmol, 1 equiv.) in MeOH/H₂O (4:1, 50 mL), was added NaOH (1.6 g, 38.8 mmol, 5 equiv.). The resulting mixture was stirred at room temperature for 4 h. Once complete consumption of starting material was observed (TLC analysis) the mixture was then acidified with 1 M HCl to pH˜3 and extracted with EtOAc (3×35 mL). The combined organic extract was dried over Na₂SO₄ and concentrated to dryness under vacuum. The crude carboxylic acid product was used in the subsequent step without further purification.

Step e: To a solution of the product from step d (5.3 g, 18.2 mmol, 1.0 equiv.), 1-amino-3-methylthiourea (2.30 g, 21.84 mmol, 1.2 equiv.) and HATU (9.7 g, 26.0 mmol, 1.4 equiv.) in THF (60 mL) was added DIPEA (9.5 mL, 54.6 mmol, 3.0 equiv.). The resulting mixture was stirred at room temperature for 12 h before being quenched with H₂O (100 mL). The mixture was then diluted with EtOAc (100 mL). The organic phase was separated, and the aqueous layer was additionally extracted with EtOAc (2×30 mL). The combined organic phase was dried over Na₂SO₄, and concentrated. The crude product was directly used in the next step without purification.

Step f: The product from step e (6.1 g, 16.1 mmol) was treated with aq. 1 M NaOH solution (160 mL) for 24 h at 75° C. The reaction mixture was cooled to room temperature, acidified to pH˜1 with 1 M HCl, and extracted with EtOAc (3×70 mL). The combined organic phase was washed with brine, dried over Na₂SO₄, and concentrated to dryness under reduced pressure. The dry residue was suspended in CH₂Cl₂ (72 mL) and acetic acid (9 mL). The reaction was cooled to 0° C. and hydrogen peroxide (5 mL) was added. The reaction was allowed to warm to room temperature and stirred for 3 h. The reaction was diluted with aq. 1 M NaHCO₃ to neutralize acetic acid, and the product was extracted with CH₂Cl₂ (3×40 mL). The combined organic phase was dried over Na₂SO₄ and concentrated to dryness under reduced pressure. The crude material was purified by reverse phase column chromatography (C18 modified silica gel, 10-100% acetonitrile gradient in water with 0.1% formic acid) to afford the desired triazole product.

Step g: A 40 mL vial was charged with the product from step f (1.0 g, 3 mmol, 1.0 equiv.), tert-butyl carbamate (0.42 g, 3.6 mmol, 1.2 equiv.), and Cs₂CO₃ (1.9 g, 6.0 mmol, 2.0 equiv.). The reagents were dissolved in dioxane (15 mL, 0.2 M) and the reaction mixture was sparged with N₂ for 10 minutes. Pd₂(dba)₃ (0.14 g, 0.15 mmol, 0.05 equiv) and XantPhos (0.17 g, 0.3 mmol, 0.1 equiv.) were added and the reaction was heated to 90° C. under stirring for 12 h. The reaction mixture was cooled to room temperature, diluted with EtOAc (100 mL) and washed with water (50 mL) and brine (50 mL). The organic solution was dried over Na₂SO₄, filtered, and concentrated to dryness under vacuum. The crude product was directly used in the next step without purification.

Step h: To a solution of the crude product from step g in CH₂C₂ (3 mL) was added trifluoroacetic acid (1 ml). The resulting solution was stirred at room temperature for 3 h. The solvent was removed under vacuum, and the resulting residue was purified via silica gel flash column chromatography (SiO₂, 0-20% MeOH in dichloromethane) to afford 6-cyclopropyl-4-[4-fluoro-2-(4-methyl-1,2,4-triazol-3-yl)phenyl]pyridin-2-amine.

Step i: To a solution of the methyl 1-cyclopropyl-5-formyl-2-oxopyridine-3-carboxylate (1.0 g, 4.5 mmol, 1 equiv., prepared according to Example 1) in MeOH/THF mixture (45 mL, 1:1 v/v), was added aq. 1 M LiOH solution (5.4 mL, 1.2 equiv.). The resulting mixture was stirred at room temperature for 12 h, then acidified with 1 M HCl to pH˜1, and the product was extracted with EtOAc (3×20 mL). The combined organic extract was dried over Na₂SO₄ and concentrated to dryness under vacuum. The crude carboxylic acid product was used in the next step without further purification.

Step j: To a solution of the product from step i (401.8 mg, 1.93 mmol, 1.2 equiv.), 6-cyclopropyl-4-[4-fluoro-2-(4-methyl-1,2,4-triazol-3-yl)phenyl]pyridin-2-amine (0.5 g, 1.61 mmol, 1.0 equiv.) and HATU (0.67 g, 1.77 mmol, 1.1 equiv.) in DMF (5 mL) was added DIPEA (0.84 mL, 4.8 mmol, 3.0 equiv.). The resulting mixture was left to stir at room temperature overnight. The resulting mixture was directly fractionated by reverse phase column chromatography (C18 modified silica gel, 10-100% acetonitrile gradient in water with 0.1% formic acid) to afford 1-cyclopropyl-N-[6-cyclopropyl-4-[4-fluoro-2-(4-methyl-1,2,4-triazol-3-yl)phenyl]pyridin-2-yl]-5-formyl-2-oxopyridine-3-carboxamide as the desired product.

Step k: To a stirred solution of 1-cyclopropyl-N-[6-cyclopropyl-4-[4-fluoro-2-(4-methyl-1,2,4-triazol-3-yl)phenyl]pyridin-2-yl]-5-formyl-2-oxopyridine-3-carboxamide (40 mg, 0.08 mmol, 1.0 equiv) and 3,3,3-trifluoropropylamine (18.0 mg, 0.16 mmol, 2.0 equiv.) in THF (0.8 mL, 0.1 M) was added Ti(Oi-Pr)₄ (113 mg, 0.4 mmol, 5.0 equiv.) at room temperature. The obtained mixture was stirred at 60° C. for 3 h, then cooled to room temperature, and NaBH₃CN (15 mg, 0.24 mmol, 3.0 equiv) was added. After stirring for 2 h the reaction was quenched with MeOH (1 mL) and concentrated to dryness under reduced pressure. The dry residue was partitioned between CH₂Cl₂ (20 mL) and aq. sat. NaHCO₃ (10 mL). Organic phase was separated, and the aqueous phase was additionally extracted with CH₂Cl₂ (2×10 mL). The combined organic extract was washed with water, dried over Na₂SO₄, filtered, and the solvent was removed under reduced pressure. The crude product was purified by reversed phase preparative HPLC (20-90% CH₃CN in water with 0.1% TFA) to afford the title compound. ¹H NMR (400 MHz, CDCl₃) δ 12.22 (s, 1H), 8.53 (d, J=2.6 Hz, 1H), 8.13 (d, J=1.4 Hz, 1H), 8.04 (s, 1H), 7.67-7.57 (m, 2H), 7.41-7.29 (m, 2H), 6.49 (d, J=1.5 Hz, 1H), 3.68 (s, 2H), 3.49-3.41 (m, 1H), 3.09 (s, 3H), 2.92 (t, J=7.0 Hz, 2H), 2.43-2.26 (m, 2H), 1.84-1.76 (m, 2H), 1.28-1.22 (m, 2H), 0.97-0.92 (m, 2H), 0.91-0.83 (m, 4H). ESI MS [M+H]+ for C₃₀H₂₉F₄N₇O₂, calcd 596.2, found 596.2.

### Example 16: N-{6-cyclopropyl-4-[4-fluoro-2-(4-methyl-4H-1,2,4-triazol-3-yl)phenyl]-2-pyridyl}-5-{[(S)-3-methoxy-1-pyrrolidinyl]methyl}-1-cyclopropyl-2-oxo-1,2-dihydronicotinamide

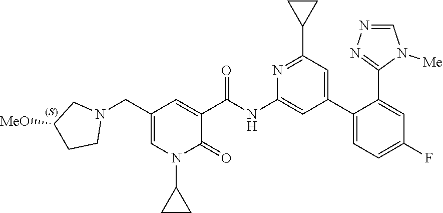

The title compound was prepared in a similar fashion to that described for Example 15 using (3S)-3-methoxypyrrolidine hydrochloride in step k. ¹H NMR (400 MHz, CDCl₃) δ 12.23 (s, 1H), 8.51 (d, J=2.7 Hz, 1H), 8.17 (d, J=1.5 Hz, 1H), 8.04 (s, 1H), 7.68-7.60 (m, 2H), 7.39-7.30 (m, 2H), 6.45 (d, J=1.5 Hz, 1H), 3.99-3.90 (m, 1H), 3.51 (s, 2H), 3.49-3.41 (m, 1H), 3.29 (s, 3H), 3.08 (s, 3H), 2.82-2.70 (m, 2H), 2.66-2.51 (m, 2H), 2.16-2.05 (m, 1H), 1.90-1.76 (m, 2H), 1.29-1.21 (m, 2H), 1.00-0.92 (m, 2H), 0.91-0.83 (m, 4H). ESI MS [M+H]+ for C₃₂H₃₄FN₇O₃, calcd 584.2, found 584.3.

### Example 17: N-{6-cyclopropyl-4-[4-fluoro-2-(4-methyl-4H-1,2,4-triazol-3-yl)phenyl]-2-pyridyl}-5-{[(S)-2-methoxypropylamino]methyl}-1-cyclopropyl-2-oxo-1,2-dihydronicotinamide

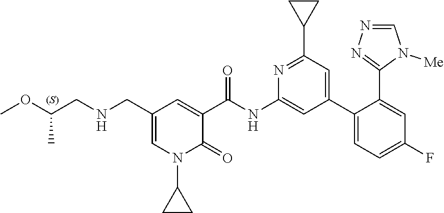

The title compound was prepared in a similar fashion to that described for Example 15 using (2S)-2-methoxypropan-1-amine hydrochloride in step k. ¹H NMR (400 MHz, CDCl₃) δ 12.24 (s, 1H), 8.52 (d, J=2.6 Hz, 1H), 8.13 (d, J=1.4 Hz, 1H), 8.05 (s, 1H), 7.71 (d, J=2.6 Hz, 1H), 7.64 (dd, J=8.5, 5.5 Hz, 1H), 7.39-7.30 (m, 2H), 6.48 (d, J=1.4 Hz, 1H), 3.69 (s, 2H), 3.55-3.40 (m, 2H), 3.34 (s, 3H), 3.10 (s, 3H), 2.72-2.60 (m, 2H), 2.52 (s, 1H), 1.85-1.77 (m, 1H), 1.27-1.20 (m, 2H), 1.14 (d, J=6.2 Hz, 3H), 1.00-0.94 (m, 2H), 0.91-0.84 (m, 4H). ESI MS [M+H]+ for C₃₁H₃₄FN₇O₃, calcd 572.2, found 572.2.

### Example 18: N-{6-cyclopropyl-4-[4-fluoro-2-(4-methyl-4H-1,2,4-triazol-3-yl)phenyl]-2-pyridyl}-5-{[(S)-2-methoxy-1-methylethylamino]methyl}-1-cyclopropyl-2-oxo-1,2-dihydronicotinamide

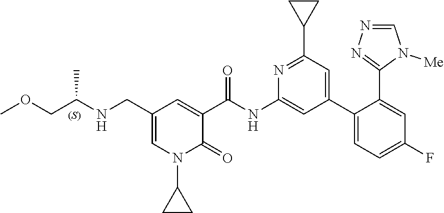

The title compound was prepared in a similar fashion to that described for Example 15 using (2S)-1-methoxypropan-2-amine hydrochloride in step k. ¹H NMR (400 MHz, CDCl₃) δ 12.25 (s, 1H), 8.53 (d, J=2.6 Hz, 1H), 8.15 (d, J=1.5 Hz, 1H), 8.04 (s, 1H), 7.69 (d, J=2.6 Hz, 1H), 7.63 (dd, J=8.6, 5.4 Hz, 1H), 7.40-7.30 (m, 2H), 6.47 (d, J=1.4 Hz, 1H), 3.79-3.61 (m, 2H), 3.48-3.33 (m, 5H), 3.30-3.23 (m, 1H), 3.09 (s, 3H), 3.01-2.90 (m, 1H), 1.98 (s, 1H), 1.84-1.77 (m, 1H), 1.28-1.21 (m, 2H), 1.08 (d, J=6.4 Hz, 3H), 1.00-0.94 (m, 2H), 0.91-0.83 (m, 4H). ESI MS [M+H]+ for C₃₁H₃₄FN₇O₃, calcd 572.2, found 572.2.

### Example 19: N-{6-cyclopropyl-4-[4-fluoro-2-(4-methyl-4H-1,2,4-triazol-3-yl)phenyl]-2-pyridyl}-1-cyclopropyl-5-[(2,2-difluoro-2-methoxyethylamino)methyl]-2-oxo-1,2-dihydronicotinamide

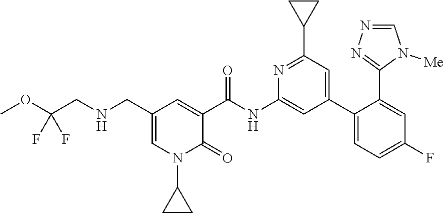 [SMILES: COC(F)(F)CNCc1cc(C(=O)Nc2cc(-c3ccc(F)cc3-c3nncn3C)cc(C3CC3)n2)c(=O)n(C2CC2)c1]

The title compound was prepared in a similar fashion to that described for Example 15 using 2,2-difluoro-2-methoxyethanamine hydrochloride in step k. ¹H NMR (400 MHz, CDCl₃) δ 12.23 (s, 1H), 8.53 (d, J=2.7 Hz, 1H), 8.16-8.12 (m, 1H), 8.04 (s, 1H), 7.67-7.59 (m, 2H), 7.40-7.29 (m, 2H), 6.48 (d, J=1.4 Hz, 1H), 3.76 (s, 2H), 3.59 (s, 3H), 3.49-3.42 (m, 1H), 3.11-3.02 (m, 5H), 1.94-1.76 (m, 2H), 1.30-1.22 (m, 2H), 0.99-0.93 (m, 2H), 0.90-0.84 (m, 4H). ESI MS [M+H]+ for C₃₀H₃₀F₃N₇O₃, calcd 594.2, found 594.2.

### Example 20: N-{6-cyclopropyl-4-[4-fluoro-2-(4-methyl-4H-1,2,4-triazol-3-yl)phenyl]-2-pyridyl}-5-{[(R)-2-methoxypropylamino]methyl}-1-cyclopropyl-2-oxo-1,2-dihydronicotinamide

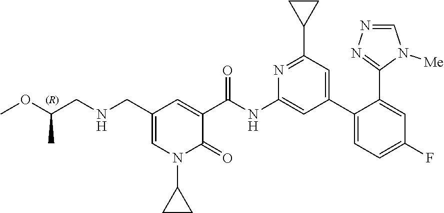

The title compound was prepared in a similar fashion to that described for Example 15 using (2R)-2-methoxypropan-1-amine hydrochloride in step k. ¹H NMR (400 MHz, CDCl₃) δ 12.24 (s, 1H), 8.53 (d, J=2.6 Hz, 1H), 8.14 (d, J=1.5 Hz, 1H), 8.05 (s, 1H), 7.72-7.58 (m, 2H), 7.41-7.31 (m, 2H), 6.48 (d, J=1.5 Hz, 1H), 3.76-3.63 (m, 2H), 3.55-3.39 (m, 2H), 3.34 (s, 3H), 3.10 (s, 3H), 2.73-2.60 (m, 2H), 2.13 (s, 1H), 1.86-1.78 (m, 1H), 1.29-1.22 (m, 2H), 1.15 (d, J=6.1 Hz, 3H), 0.99-0.93 (m, 2H), 0.92-0.80 (m, 4H). ESI MS [M+H]+ for C₃₁H₃₄FN₇O₃, calcd 572.2, found 572.2.

### Example 21: N-{6-cyclopropyl-4-[4-fluoro-2-(4-methyl-4H-1,2,4-triazol-3-yl)phenyl]-2-pyridyl}-5-{[(R)-2-methoxy-1-methylethylamino]methyl}-1-cyclopropyl-2-oxo-1,2-dihydronicotinamide

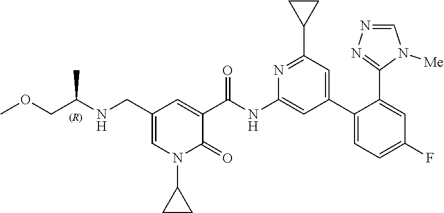

The title compound was prepared in a similar fashion to that described for Example 15 using (2R)-1-methoxypropan-2-amine in step k. ¹H NMR (400 MHz, CDCl₃) δ 12.25 (s, 1H), 8.53 (d, J=2.7 Hz, 1H), 8.15 (d, J=1.5 Hz, 1H), 8.05 (s, 1H), 7.70 (d, J=2.6 Hz, 1H), 7.63 (dd, J=8.5, 5.4 Hz, 1H), 7.41-7.29 (m, 2H), 6.47 (d, J=1.5 Hz, 1H), 3.81-3.63 (m, 2H), 3.48-3.33 (m, 5H), 3.31-3.24 (m, 1H), 3.10 (s, 3H), 3.03-2.93 (m, 1H), 2.24 (s, 1H), 1.86-1.76 (m, 1H), 1.28-1.20 (m, 2H), 1.09 (d, J=6.5 Hz, 3H), 1.00-0.94 (m, 2H), 0.91-0.82 (m, 4H). ESI MS [M+H]+ for C₃₁H₃₄FN₇O₃, calcd 572.2, found 572.2.

### Example 22: N-{6-cyclopropyl-4-[4-fluoro-2-(4-methyl-4H-1,2,4-triazol-3-yl)phenyl]-2-pyridyl}-5-{[(R)-3-methoxy-1-pyrrolidinyl]methyl}-1-cyclopropyl-2-oxo-1,2-dihydronicotinamide

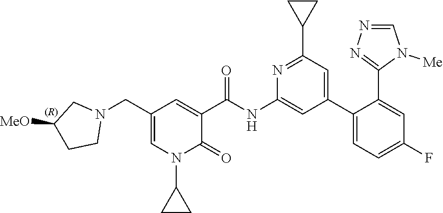

The title compound was prepared in a similar fashion to that described for Example 15 using (3R)-3-methoxypyrrolidine in step k. ¹H NMR (400 MHz, CDCl₃) δ 12.23 (s, 1H), 8.51 (d, J=2.7 Hz, 1H), 8.15 (d, J=1.5 Hz, 1H), 8.04 (s, 1H), 7.73-7.59 (m, 2H), 7.42-7.29 (m, 2H), 6.47 (d, J=1.5 Hz, 1H), 4.01-3.92 (m, 1H), 3.60-3.39 (m, 3H), 3.29 (s, 3H), 3.09 (s, 3H), 2.86-2.72 (m, 2H), 2.70-2.53 (m, 2H), 2.16-2.03 (m, 1H), 1.93-1.77 (m, 2H), 1.28-1.21 (m, 2H), 1.02-0.93 (m, 2H), 0.94-0.81 (m, 4H). ESI MS [M+H]+ for C₃₂H₃₄FN₇O₃, calcd 584.2, found 584.2.

### Example 23: N-{6-cyclopropyl-4-[4-fluoro-2-(4-methyl-4H-1,2,4-triazol-3-yl)phenyl]-2-pyridyl}-5-{[(1S,2S)-2-methoxy-1-methylpropylamino]methyl}-1-cyclopropyl-2-oxo-1,2-dihydronicotinamide

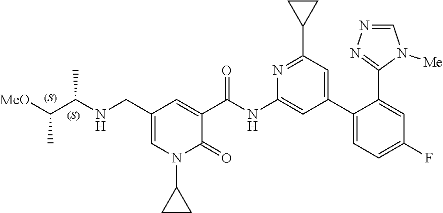

The title compound was prepare in a similar fashion to that described for Example 15 using (2S,3S)-3-methoxybutan-2-amine in step k. ¹H NMR (400 MHz, CDCl₃) δ 12.25 (s, 1H), 8.53 (d, J=2.6 Hz, 1H), 8.16 (d, J=1.5 Hz, 1H), 8.05 (s, 1H), 7.69 (d, J=2.6 Hz, 1H), 7.66-7.61 (m, 1H), 7.41-7.29 (m, 2H), 6.46 (d, J=1.5 Hz, 1H), 3.74 (d, J=13.4 Hz, 1H), 3.58 (d, J=13.5 Hz, 1H), 3.50-3.42 (m, 1H), 3.34 (s, 3H), 3.21-3.04 (m, 4H), 2.72-2.58 (m, 1H), 2.09-1.77 (m, 2H), 1.32-1.21 (m, 2H), 1.14 (d, J=6.2 Hz, 3H), 1.07 (d, J=6.4 Hz, 3H), 1.00-0.81 (m, 6H). ESI MS [M+H]+ for C₃₂H₃₆FN₇O₃, calcd 586.2, found 586.2.

### Example 24: N-{6-cyclopropyl-4-[4-fluoro-2-(4-methyl-4H-1,2,4-triazol-3-yl)phenyl]-2-pyridyl}-1-cyclopropyl-5-({[(1-fluorocyclobutyl)methyl]amino}methyl)-2-oxo-1,2-dihydronicotinamide

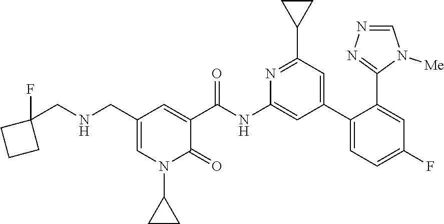 [SMILES: Cn1cnnc1-c1cc(F)ccc1-c1cc(NC(=O)c2cc(CNCC3(F)CCC3)cn(C3CC3)c2=O)nc(C2CC2)c1]

The title compound was prepared in a similar fashion to that described for Example 15 using (1-fluorocyclobutyl)methanamine hydrochloride in step k. ¹H NMR (400 MHz, CDCl₃) δ 12.25 (s, 1H), 8.55 (d, J=2.7 Hz, 1H), 8.15 (d, J=1.5 Hz, 1H), 8.04 (s, 1H), 7.71-7.59 (m, 2H), 7.41-7.30 (m, 2H), 6.47 (d, J=1.5 Hz, 1H), 3.72 (s, 2H), 3.52-3.38 (m, 1H), 3.09 (s, 3H), 2.94-2.78 (m, 2H), 2.40-2.12 (m, 4H), 1.91-1.70 (m, 3H), 1.53-1.43 (m, 1H), 1.30-1.20 (m, 2H), 0.99-0.79 (m, 6H). ESI MS [M+H]+ for C₃₂H₃₃F₂N₇O₂, calcd 586.2, found 586.2.

### Example 25: N-{6-cyclopropyl-4-[4-fluoro-2-(4-methyl-4H-1,2,4-triazol-3-yl)phenyl]-2-pyridyl}-5-{[(S)-3-methoxy-1-piperidyl]methyl}-1-cyclopropyl-2-oxo-1,2-dihydronicotinamide

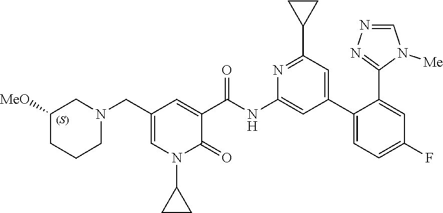

The title compound was prepared in a similar fashion to that described for Example 15 using (3S)-3-methoxypiperidine hydrochloride in step k. ¹H NMR (400 MHz, CDCl₃) δ 12.24 (s, 1H), 8.52 (d, J=2.7 Hz, 1H), 8.17 (d, J=1.5 Hz, 1H), 8.04 (s, 1H), 7.67-7.57 (m, 2H), 7.41-7.29 (m, 2H), 6.45 (d, J=1.5 Hz, 1H), 3.49-3.37 (m, 3H), 3.34 (s, 4H), 3.08 (s, 3H), 2.91-2.83 (m, 1H), 2.68-2.60 (m, 1H), 2.20-2.02 (m, 1H), 1.94-1.89 (m, 2H), 1.84-1.72 (m, 2H), 1.58-1.47 (m, 1H), 1.29-1.23 (m, 3H), 1.00-0.93 (m, 2H), 0.92-0.84 (m, 4H). ESI MS [M+H]+ for C₃₃H₃₆FN₇O₃, calcd 598.2, found 586.3.

### Example 26: N-{6-cyclopropyl-4-[4-fluoro-2-(4-methyl-4H-1,2,4-triazol-3-yl)phenyl]-2-pyridyl}-1-cyclopropyl-5-[(3-methoxy-1-azetidinyl)methyl]-2-oxo-1,2-dihydronicotinamide

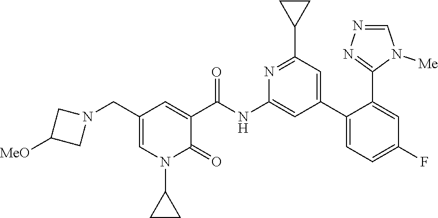 [SMILES: COC1CN(Cc2cc(C(=O)Nc3cc(-c4ccc(F)cc4-c4nncn4C)cc(C4CC4)n3)c(=O)n(C3CC3)c2)C1]

The title compound was prepared in a similar fashion to that described in Example 15 using 3-methoxyazetidine hydrochloride in step k. ¹H NMR (400 MHz, CDCl₃) δ 12.20 (s, 1H), 8.47 (d, J=2.7 Hz, 1H), 8.13 (d, J=1.5 Hz, 1H), 8.05 (s, 1H), 7.67-7.59 (m, 2H), 7.39-7.30 (m, 2H), 6.48 (d, J=1.5 Hz, 1H), 4.11-4.03 (m, 1H), 3.70-3.64 (m, 2H), 3.54 (s, 2H), 3.48-3.43 (m, 1H), 3.27 (s, 3H), 3.13-2.99 (m, 5H), 1.84-1.76 (m, 1H), 1.28-1.23 (m, 2H), 0.99-0.93 (m, 2H), 0.92-0.83 (m, 4H). ESI MS [M+H]+ for C₃₁H₃₂FN₇O₃, calcd 570.2, found 570.2.

### Example 27: N-{6-Cyclopropyl-4-[4-fluoro-2-(4-methyl-4H-1,2,4-triazol-3-yl)phenyl]-2-pyridyl}-1-cyclopropyl-2-oxo-5-(3-pyrazolyl)-1,2-dihydronicotinamide

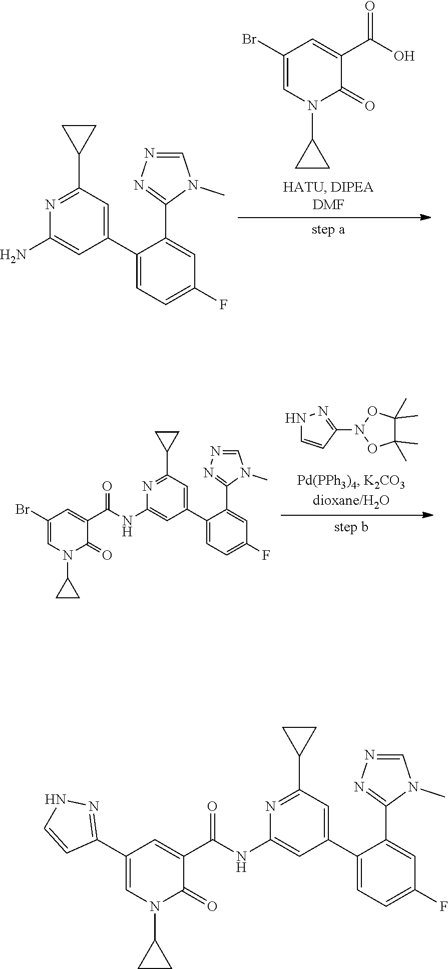 [SMILES: Cn1cnnc1-c1cc(F)ccc1-c1cc(N)nc(C2CC2)c1 ; O=C(O)c1cc(Br)cn(C2CC2)c1=O ; Cn1cnnc1-c1cc(F)ccc1-c1cc(NC(=O)c2cc(Br)cn(C3CC3)c2=O)nc(C2CC2)c1 ; CC1(C)ON(c2cc[nH]n2)OC1(C)C ; Cn1cnnc1-c1cc(F)ccc1-c1cc(NC(=O)c2cc(-c3cc[nH]n3)cn(C3CC3)c2=O)nc(C2CC2)c1]

Step a: To a solution of 5-bromo-1-cyclopropyl-2-oxopyridine-3-carboxylic acid (0.26 g, 1.0 mmol, 1.0 equiv.) and 6-cyclopropyl-4-[4-fluoro-2-(4-methyl-1,2,4-triazol-3-yl)phenyl]pyridin-2-amine (371 mg, 1.2 mmol, 1.2 equiv., prepared according to protocol described for Example 15) in DMF (4 mL) was added HATU (0.46 g, 1.2 mmol, 1.2 equiv.) and diisopropylethylamine (0.35 mL, 259 mg, 2.0 mmol, 2.0 equiv.). The resulting mixture was stirred at room temperature for 1.5 h and then concentrated under vacuum. The crude residue was fractionated by column chromatography (SiO₂, 0-10% MeOH gradient in CH₂Cl₂) to afford corresponding amide product.

Step b: To a mixture of amide product from step a (40.0 mg, 0.070 mmol, 1.0 equiv.) and 3-(4,4,5,5-tetramethyl-1,3,2-dioxaborolan-2-yl)-1H-pyrazole (20 mg, 0.10 mmol, 1.5 equiv.) in dioxane (0.70 mL) was added Pd(PPh₃)₄ (8 mg, 0.007 mmol, 10 mol %) and K₂CO₃ (1 M in H₂O, 0.14 mL, 2.0 equiv.). The resulting mixture was heated at 100° C. under N₂ for 2 h before being cooled to room temperature and diluted with EtOAc (10 mL) and water (10 mL). The organic phase was separated, and the aqueous layer was additionally extracted with EtOAc (2×10 mL). The combined organic extract was dried over Na₂SO₄ and concentrated. The crude residue was then purified by reversed phase preparative HPLC to afford the title compound. ¹H NMR (400 MHz, DMSO-d₆) δ 12.95 (s, 1H), 12.38 (s, 1H), 8.89 (s, 1H), 8.51 (s, 1H), 8.30 (s, 1H), 7.81 (s, 1H), 7.77 (d, J=1.4 Hz, 1H), 7.73 (dd, J=8.6, 5.6 Hz, 1H), 7.67-7.52 (m, 2H), 6.80 (s, 1H), 6.73 (d, J=1.4 Hz, 1H), 3.53 (tt, J=7.1, 4.3 Hz, 1H), 3.36 (s, 3H), 1.98 (td, J=8.2, 4.3 Hz, 1H), 1.22-1.04 (m, 4H), 0.93 (dt, J=8.1, 3.1 Hz, 2H), 0.89-0.77 (m, 2H). ESI MS [M+H]+ for C₂₉H₂₅FN₈O₂, calcd 537.2, found 537.2.

### Example 28: N-{6-Cyclopropyl-4-[4-fluoro-2-(4-methyl-4H-1,2,4-triazol-3-yl)phenyl]-2-pyridyl}-5-(m-aminophenyl)-1-cyclopropyl-2-oxo-1,2-dihydronicotinamide

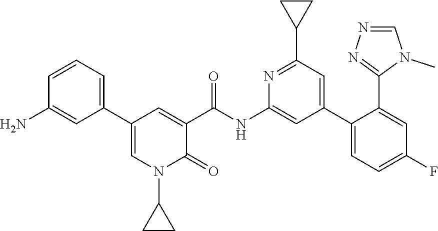 [SMILES: Cn1cnnc1-c1cc(F)ccc1-c1cc(NC(=O)c2cc(-c3cccc(N)c3)cn(C3CC3)c2=O)nc(C2CC2)c1]

The title compound was synthesized according to the procedure described for the Example 27 using 3-(4,4,5,5-tetramethyl-1,3,2-dioxaborolan-2-yl)aniline in step b. ¹H NMR (400 MHz, DMSO-d₆) δ 12.38 (s, 1H), 8.61 (d, J=2.8 Hz, 1H), 8.49 (s, 1H), 8.11 (d, J=2.8 Hz, 1H), 7.78 (d, J=1.5 Hz, 1H), 7.73 (dd, J=8.6, 5.7 Hz, 1H), 7.65-7.52 (m, 2H), 7.11 (t, J=7.8 Hz, 1H), 6.80 (t, J=2.0 Hz, 1H), 6.77 (dt, J=7.6, 1.3 Hz, 1H), 6.73 (d, J=1.5 Hz, 1H), 6.56 (ddd, J=8.0, 2.2, 1.0 Hz, 1H), 5.22 (s, 2H), 3.52 (ddd, J=11.6, 7.4, 4.5 Hz, 1H), 3.33 (s, 3H), 1.98 (td, J=8.2, 4.2 Hz, 1H), 1.16-1.03 (m, 4H), 0.94 (dt, J=7.9, 3.1 Hz, 2H), 0.84 (dt, J=5.0, 3.0 Hz, 2H). ESI MS [M+H]⁺ for C₃₂H₂₈FN₇O₂, calcd 562.2, found 562.2.

### Example 29: N-{6-Cyclopropyl-4-[4-fluoro-2-(4-methyl-4H-1,2,4-triazol-3-yl)phenyl]-2-pyridyl}-1-cyclopropyl-5-cyclopropyl-2-oxo-1,2-dihydronicotinamide

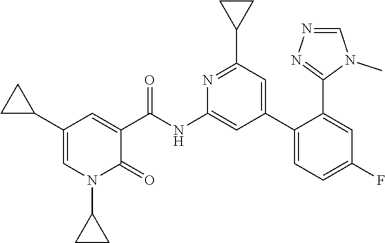 [SMILES: Cn1cnnc1-c1cc(F)ccc1-c1cc(NC(=O)c2cc(C3CC3)cn(C3CC3)c2=O)nc(C2CC2)c1]

The title compound was synthesized according to the procedure described for the Example 27 using cyclopropylboronic acid in step b. ¹H NMR (400 MHz, CDCl₃) δ 12.32 (s, 1H), 8.31 (d, J=2.8 Hz, 1H), 8.18 (d, J=1.5 Hz, 1H), 8.03 (s, 1H), 7.63 (dd, J=8.6, 5.5 Hz, 1H), 7.44 (d, J=2.8 Hz, 1H), 7.40-7.30 (m, 2H), 6.44 (d, J=1.5 Hz, 1H), 3.43 (tt, J=7.6, 4.2 Hz, 1H), 3.07 (s, 3H), 1.77 (dtt, J=18.4, 8.4, 5.2 Hz, 2H), 1.31-1.22 (m, 2H), 1.00-0.91 (m, 2H), 0.89-0.83 (m, 2H), 0.62 (dt, J=6.4, 5.0 Hz, 2H). ESI MS [M+H]⁺ for C₂₉H₂₇FN₆O₂, calcd 511.2, found 511.1.

### Example 30: N-{6-Cyclopropyl-4-[4-fluoro-2-(4-methyl-4H-1,2,4-triazol-3-yl)phenyl]-2-pyridyl}-1-cyclopropyl-2-oxo-5-(3-pyrrolidinyl)-1,2-dihydronicotinamide

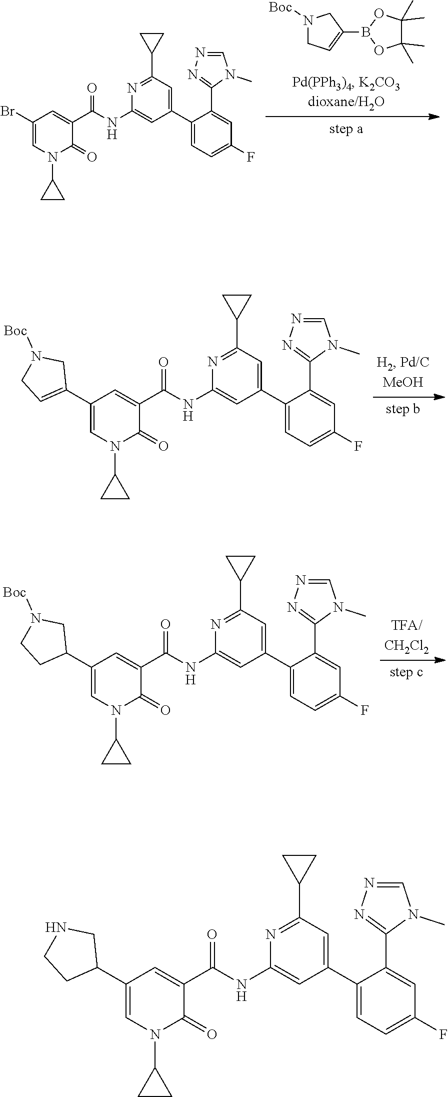 [SMILES: Cn1cnnc1-c1cc(F)ccc1-c1cc(NC(=O)c2cc(Br)cn(C3CC3)c2=O)nc(C2CC2)c1 ; CC(C)(C)OC(=O)N1CC=C(B2OC(C)(C)C(C)(C)O2)C1 ; Cn1cnnc1-c1cc(F)ccc1-c1cc(NC(=O)c2cc(C3=CCN(C(=O)OC(C)(C)C)C3)cn(C3CC3)c2=O)nc(C2CC2)c1 ; Cn1cnnc1-c1cc(F)ccc1-c1cc(NC(=O)c2cc(C3CCN(C(=O)OC(C)(C)C)C3)cn(C3CC3)c2=O)nc(C2CC2)c1 ; Cn1cnnc1-c1cc(F)ccc1-c1cc(NC(=O)c2cc(C3CCNC3)cn(C3CC3)c2=O)nc(C2CC2)c1]

Step a was performed according to protocol described for Example 27 using tert-butyl 3-(4,4,5,5-tetramethyl-1,3,2-dioxaborolan-2-yl)-2,5-dihydropyrrole-1-carboxylate in step b.

Step b: The product of step a (31.1 mg, 0.050 mmol), Pd/C (30 mg, 10 wt % Pd) and MeOH (5 mL) were loaded in a parr shaker glass reactor. The resulting mixture was agitated under an atmosphere of hydrogen (50 psi) overnight. The obtained suspension was filtered through a pad of CELITE® and concentrated to dryness under reduced pressure to afford the desired product.

Step c: To the solution of the crude product from step b (0.050 mmol) in CH₂Cl₂ (1 mL) was added trifluoroacetiuc acid (0.20 mL). The resulting mixture was stirred at room temperature for 2 h and concentrated to dryness. The crude product was purified by reversed phase preparative HPLC to afford the title compound. ¹H NMR (400 MHz, DMSO-d₆) δ 12.35 (s, 1H), 8.42 (s, 1H), 8.34 (d, J=2.7 Hz, 1H), 7.87-7.80 (m, 1H), 7.71 (d, J=1.4 Hz, 1H), 7.63 (dd, J=8.6, 5.6 Hz, 1H), 7.57-7.43 (m, 2H), 6.63 (d, J=1.5 Hz, 1H), 3.39 (td, J=7.5, 3.9 Hz, 1H), 3.24 (s, 3H), 3.15-3.04 (m, 3H), 2.93 (dt, J=10.7, 7.8 Hz, 1H), 2.72 (dd, J=10.4, 8.2 Hz, 1H), 2.16-2.03 (m, 1H), 1.90 (td, J=8.3, 4.2 Hz, 1H), 1.67 (dq, J=12.4, 8.6 Hz, 1H), 1.08-0.97 (m, 2H), 0.97-0.89 (m, 2H), 0.89-0.82 (m, 2H), 0.82-0.68 (m, 2H). ESI MS [M+H]⁺ for C₃₀H₃₀FN₇O₂, calcd 540.3, found 540.2.

### Example 31: N-[6-cyclopropyl-4-[4-fluoro-2-(4-methyl-1,2,4-triazol-3-yl)phenyl]pyridin-2-yl]-1-(cyclopropylmethyl)-2-oxo-5-pyrrolidin-3-ylpyridine-3-carboxamide

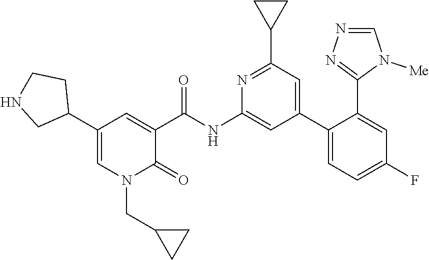 [SMILES: Cn1cnnc1-c1cc(F)ccc1-c1cc(NC(=O)c2cc(C3CCNC3)cn(CC3CC3)c2=O)nc(C2CC2)c1]

The title compound was prepared in a similar fashion to that described for Example 30, using the product of Example 56, step c as the starting material. ¹H NMR (400 MHz, DMSO-d₆) δ 12.36 (s, 1H), 8.42 (s, 1H), 8.36 (d, J=2.5 Hz, 1H), 8.23 (s, 1H), 8.07 (s, 1H), 7.71 (d, J=1.5 Hz, 1H), 7.64 (dd, J=8.5, 5.8 Hz, 1H), 7.57-7.45 (m, 2H), 6.62 (d, J=1.5 Hz, 1H), 3.86 (d, J=7.2 Hz, 2H), 3.24 (s, 3H), 3.18-3.07 (m, 3H), 3.02-2.88 (m, 1H), 2.86-2.70 (m, 1H), 2.21-2.07 (m, 1H), 1.99-1.83 (m, 1H), 1.77-1.64 (m, 1H), 1.34-1.17 (m, 1H), 0.86 (d, J=7.7 Hz, 2H), 0.73 (d, J=3.6 Hz, 2H), 0.49-0.34 (m, 4H),. ESI MS [M+H]⁺ for C₃₁H₃₃FN₇O₂, calcd 554.3, found 554.2.

### Example 32: N-{6-Cyclopropyl-4-[4-fluoro-2-(4-methyl-4H-1,2,4-triazol-3-yl)phenyl]-2-pyridyl}-5-[(dimethylamino)methyl]-2-oxo-1,2-dihydronicotinamide

### Example 33: N-{6-Cyclopropyl-4-[4-fluoro-2-(4-methyl-4H-1,2,4-triazol-3-yl)phenyl]-2-pyridyl}-1-cyclopropyl-5-[(dimethylamino)methyl]-2-oxo-1,2-dihydronicotinamide and

### Example 34: N-{6-Cyclopropyl-4-[4-fluoro-2-(4-methyl-4H-1,2,4-triazol-3-yl)phenyl]-2-pyridyl}-1-allyl-5-[(dimethylamino)methyl]-2-oxo-1,2-dihydronicotinamide

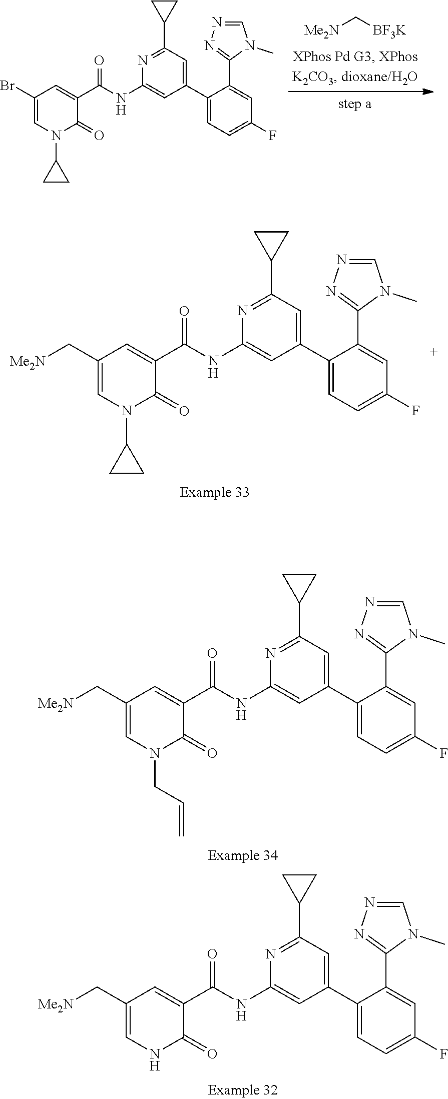

Step a: To a mixture of 5-bromo-1-cyclopropyl-N-[6-cyclopropyl-4-[4-fluoro-2-(4-methyl-1,2,4-triazol-3-yl)phenyl]pyridin-2-yl]-2-oxopyridine-3-carboxamide (54.9 mg, 0.10 mmol, 1.0 equiv., prepared according to Example 27) and potassium dimethylaminomethyltrifluoroboronate (25 mg, 0.15 mmol, 1.5 equiv.) in dioxane (1.0 mL) was added XPhos Pd G3 (9 mg, 0.010 mmol, 10 mol %), XPhos (5 mg, 0.010 mmol, 10 mol %) and K₂CO₃ (1M in H₂O, 0.20 mL, 2.0 equiv.). The resulting mixture was sparged with nitrogen for 5 min and heated at 100° C. under N₂ overnight. The resulting solution was cooled to room temperature and diluted with EtOAc (10 mL) and water (10 mL). The organic phase was separated, dried over Na₂SO₄ and concentrated to dryness under reduced pressure. The crude mixture was purified by reversed phase preparative HPLC (C18, 10-90% CH₃CN gradient in water with 0.1% formic acid) to afford separated title compounds with the order of elution represented by the order of appearance below.

Example 32: ¹H NMR (400 MHz, CDCl₃) δ 11.98 (s, 1H), 8.60 (d, J=2.6 Hz, 1H), 8.12 (s, 1H), 8.03 (d, J=1.6 Hz, 1H), 7.70 (s, 1H), 7.63 (dd, J=9.3, 5.5 Hz, 1H), 7.38-7.31 (m, 2H), 6.52 (s, 1H), 3.49 (s, 2H), 3.17 (s, 3H), 2.39 (s, 6H), 1.86 (tt, J=9.0, 5.0 Hz, 1H), 0.95-0.83 (m, 4H). ESI MS [M+H]⁺ for C₂₆H₂₆FN₇O₂, calcd 488.2, found 488.2.

Example 33: ¹H NMR (400 MHz, CDCl₃) δ 12.16 (s, 1H), 8.49 (d, J=2.7 Hz, 1H), 8.08 (s, 1H), 8.07 (s, 1H), 7.81 (s, 1H), 7.63 (dd, J=8.5, 5.4 Hz, 1H), 7.40-7.30 (m, 2H), 6.53 (s, 1H), 3.68-3.34 (m, 3H), 3.11 (s, 3H), 2.41 (s, 6H), 1.86-1.81 (m, 1H), 1.30-1.20 (m, 2H), 1.06-0.96 (m, 2H), 0.96-0.82 (m, 4H). ESI MS [M+H]⁺ for C₂₉H₃₀FN₇O₂, calcd 528.3, found 528.1.

Example 34: ¹H NMR (400 MHz, CDCl₃) δ 12.14 (s, 1H), 8.52 (d, J=2.6 Hz, 1H), 8.10 (s, 1H), 8.03 (s, 1H), 7.80 (s, 1H), 7.63 (dd, J=8.3, 5.4 Hz, 1H), 7.43-7.30 (m, 2H), 6.55 (s, 1H), 6.01 (ddt, J=16.3, 10.9, 5.8 Hz, 1H), 5.34 (d, J=10.3 Hz, 1H), 5.26 (d, J=17.2 Hz, 1H), 4.71 (d, J=5.8 Hz, 2H), 3.56 (s, 2H), 3.12 (s, 3H), 2.44 (s, 6H), 1.84 (td, J=7.8, 4.0 Hz, 1H), 0.97-0.84 (m, 4H). ESI MS [M+H]⁺ for C₂₉H₃₀FN₇O₂, calcd 528.3, found 528.2.

### Example 35: N-{6-chloro-4-[4-cyano-2-(1-methyl-2-imidazolyl)phenyl]-2-pyridyl}-5-{[(S)-3-methyl-1-piperidyl]methyl}-1-cyclopropyl-2-oxo-1,2-dihydronicotinamide

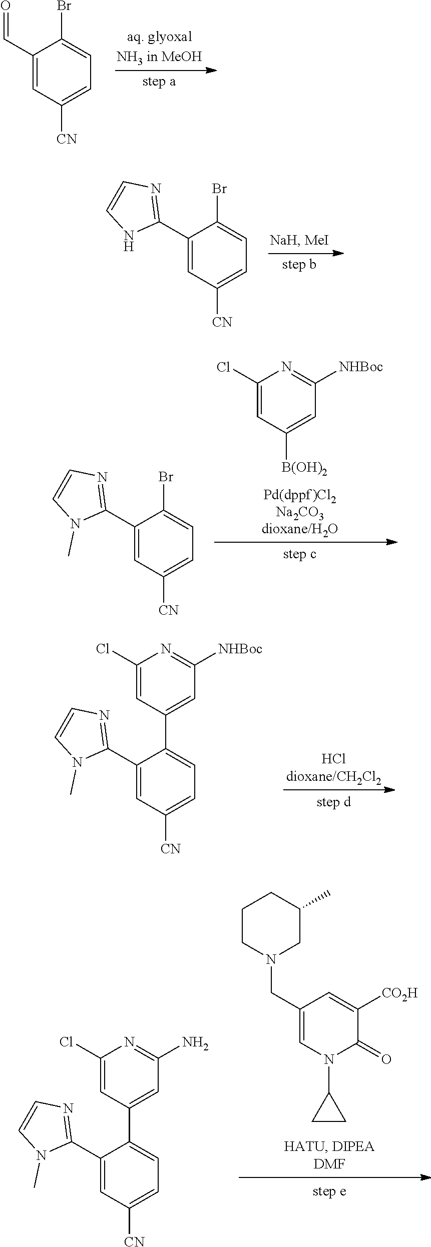

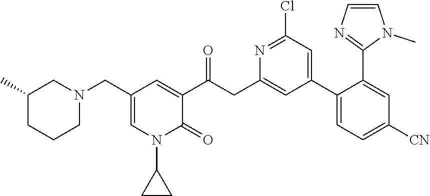

Step a: To the solution of 4-bromo-3-formylbenzonitrile (5.0 g, 9.8 mmol, 1.0 equiv.) in glyoxal aq. (3.0 mL, 29.4 mmol, 3.0 equiv.) was add NH₃ in MeOH (14.0 mL, 98 mmol, 10.0 equiv.) and the mixture was stirred at rt for 3 d. The reaction was diluted with H₂O, the organic phase was separated, and the aqueous layer was extracted with EtOAc. The combined organic phase was dried over Na₂SO₄, concentrated and the crude residue was purified by column chromatography (SiO₂, MeOH in DCM, 0 to 10%) to produce 4-bromo-3-(1H-imidazol-2-yl)benzonitrile.

Step b: To a solution of the product of step a (370 mg, 1.4919 mmol, 1.0 equiv.) in THF (10 mL, 0.15 M) was added NaH (300 mg, 7.4596 mmol, 5.0 equiv., 60% wt in oil) at 0° C. over 10 min. To the above solution was added MeI (0.7 ml, 7.4596 mmol, 5.0 equiv.). The mixture was stirred at rt for 1 h. The reaction was quenched with sat. aq. NH₄Cl solution, the organic phase was separated, and the aqueous layer was extracted with EtOAc. The combined organic phase was dried over Na₂SO₄, concentrated and the crude residue was purified by column chromatography (SiO₂, MeOH in DCM, 0 to 10%) to give 4-bromo-3-(1-methylimidazol-2-yl)benzonitrile.

Steps c-e were performed in similar fashion to that described for steps j, l, and m of example 1. ¹H NMR (400 MHz, CDCl₃) δ 12.64 (s, 1H), 8.49 (d, J=2.6 Hz, 1H), 8.33 (d, J=1.2 Hz, 1H), 7.85 (dd, J=8.1, 1.7 Hz, 1H), 7.71 (d, J=8.0 Hz, 1H), 7.65 (s, 2H), 7.13 (d, J=1.2 Hz, 1H), 6.85 (d, J=1.2 Hz, 1H), 6.59 (d, J=1.2 Hz, 1H), 3.44 (tt, J=7.7, 4.2 Hz, 1H), 3.35 (s, 2H), 3.13 (s, 3H), 2.79 (d, J=17.8 Hz, 2H), 1.94 (d, J=11.2 Hz, 1H), 1.83 (s, 3H), 1.69 (dd, J=25.2, 11.8 Hz, 3H), 1.24 (q, J=6.8 Hz, 2H), 0.95 (d, J=4.5 Hz, 2H), 0.87 (d, J=5.5 Hz, 3H). ESI MS [M+H]⁺ for C₃₂H₃₃ClN₇O₂, calcd 583.1, found 583.1.

### Example 36 and 37: N-{4-[4-cyano-2-(1-methyl-2-imidazolyl)phenyl]-6-cyclopropyl-2-pyridyl}-5-{[(R)-2-methyl-4-morpholinyl]methyl}-1-cyclopropyl-2-oxo-1,2-dihydronicotinamideand N-{4-[4-cyano-2-(1-methyl-2-imidazolyl)phenyl]-6-cyclopropyl-2-pyridyl}-1-cyclopropyl-5-(hydroxymethyl)-2-oxo-1,2-dihydronicotinamide

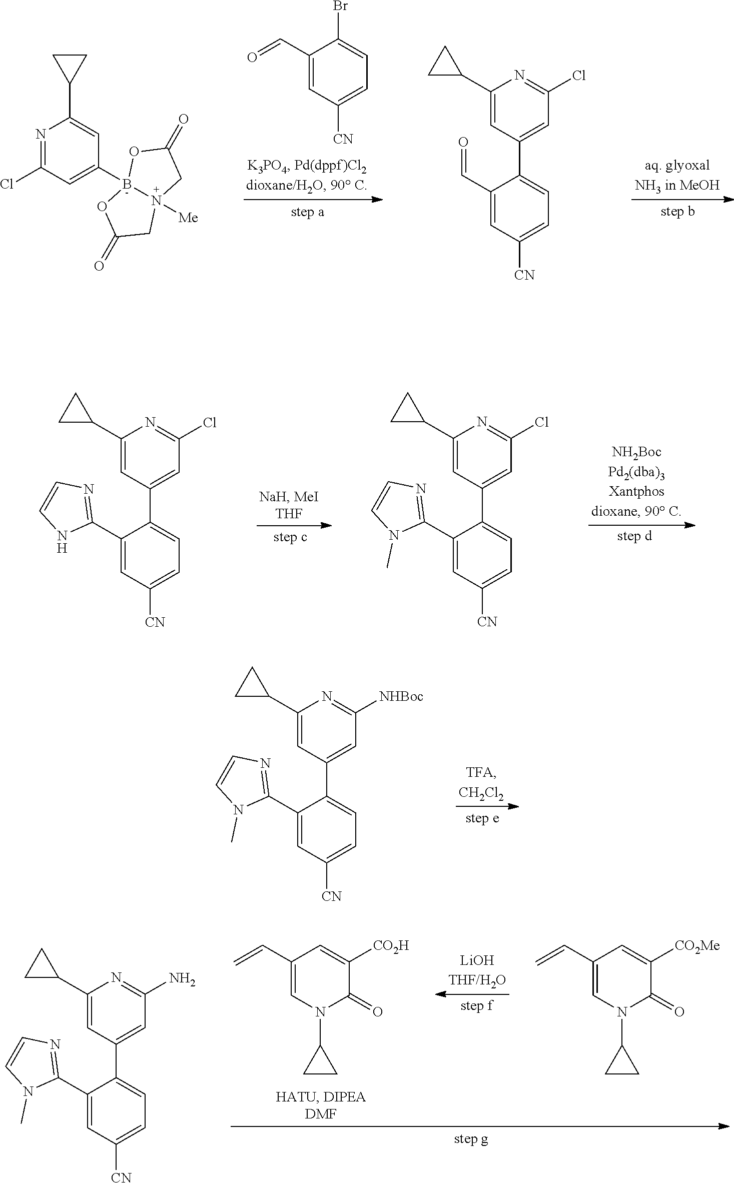

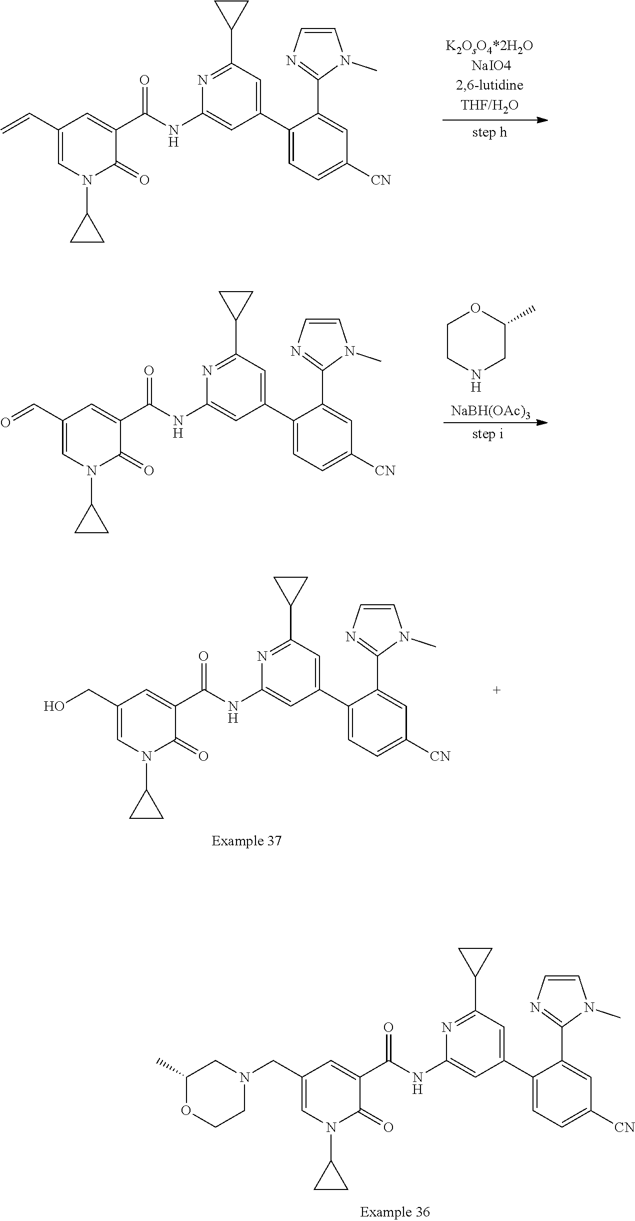

Step a: To a solution of 4-bromo-3-formylbenzonitrile (2.1 g, 10.0 mmol, 1.0 equiv.), 1-(2-chloro-6-cyclopropylpyridin-4-yl)-5-methyl-2,8-dioxa-5-azonia-1-boranuidabicyclo[3.3.0]octane-3,7-dione (3.08 g, 10.0 mmol, 1.0 equiv., prepared according to protocol described for Example 15) and K₃PO₄ (6.4 g, 30.0 mmol, 3.0 equiv.) in a dioxane water mixture (72 mL, 5:1 v/v, 0.2 M) was added Pd(dppf)Cl₂ (0.74 g, 1.0 mmol, 0.1 equiv.). The mixture was sparged with nitrogen for 10 min and stirred at 90° C. for 1 h. The resulting mixture was cooled to room temperature and diluted with sat. aq. NH₄Cl solution (50 mL), water (50 mL) and EtOAc (100 mL). The organic phase was separated, and the aqueous phase was additionally extracted with EtOAc (2×30 mL). The combined organic phase was dried over Na₂SO₄ and concentrated to dryness under reduced pressure. The crude product was purified by column chromatography (SiO₂, 0-60% EtOAc gradient in hexanes) to afford 4-(2-chloro-6-cyclopropylpyridin-4-yl)-3-(1H-imidazol-2-yl)benzonitrile.

Step b: To the solution of the product of step a (1.23 g, 4.3463 mmol, 1.0 equiv.) in glyoxal aq. (1.5 mL, 13.0389 mmol, 3.0 equiv.) was add NH₃ in MeOH (7.0 mL, 43.46 mmol, 10.0 equiv.) and the mixture was stirred at rt for 3 d. The reaction was diluted with H₂O, the organic phase was separated, and the aqueous layer was extracted with EtOAc. The combined organic phase was dried over Na₂SO₄, concentrated, and the crude residue was purified by column chromatography (SiO₂, MeOH in DCM, 0 to 10%) to produce 4-(2-chloro-6-cyclopropylpyridin-4-yl)-3-(1H-imidazol-2-yl)benzonitrile.

Step c: To a solution of the product of step b (84 mg, 0.25 mmol, 1.0 equiv.) in THF (2.5 mL, 0.1 M) was added NaH (26 mg, 0.65 mmol, 2.5 equiv., 60 wt % in oil) at 0° C. The resulting mixture was stirred for 10 min at 0° C. before MeI (111 mg, 0.79 mmol, 3.0 equiv.) was added. The reaction was allowed to warm to room temperature and stirred for 3 h. The reaction was quenched with sat. aq. NH₄Cl solution (1 mL) and diluted with EtOAc (10 mL), the organic phase was separated, and the aqueous layer was extracted with EtOAc (10 mL). The combined organic phase was dried over Na₂SO₄, concentrated to dryness, and the crude residue was purified by column chromatography (SiO₂, 0-10% MeOH gradient in dichloromethane) to yield 4-(2-chloro-6-cyclopropylpyridin-4-yl)-3-(1-methylimidazol-2-yl)benzonitrile.

Step d: To the product of step c (120 mg, 0.36 mmol, 1.0 equiv.) in dioxane (10 mL) was added tert-butyl carbamate (340 mg, 0.36 mmol, 1.1 equiv.), Pd₂(dba)₃ (33 mg, 0.018 mmol, 5 mol %) and XantPhos (42 mg, 0.036 mmol, 10 mol %). The resulting mixture was sparged with nitrogen for 10 min and heated at 90° C. for 1 h. The reaction was cooled to room temperature, the solvent was removed by vacuum, and the residue was purified by column chromatography (SiO₂, 0-10% MeOH gradient in dichloromethane) to furnish tert-butyl N-[4-[4-cyano-2-(1-methylimidazol-2-yl)phenyl]-6-cyclopropylpyridin-2-yl]carbamate.

Step e: To a solution of the crude product from step d in dichloromethane (3 ml, 0.04 M) was added TFA (1 ml, excess). The resulting solution was stirred at 23° C. for 1 h. The solvent was removed under reduced pressure, and the crude product was purified by column chromatography (SiO₂, 0-10% MeOH in CH₂Cl₂) to give 4-(2-amino-6-cyclopropylpyridin-4-yl)-3-(1-methylimidazol-2-yl)benzonitrile.

Step f: To the solution of methyl 1-cyclopropyl-5-ethenyl-2-oxopyridine-3-carboxylate (220 mg, 1.0 mmol, 1.0 equiv.) in a THF/H₂O mixture (5 ml, 4:1 v/v, 0.2 M) was added LiOH monohydrate (210 mg, 5.0 mmol, 5.0 equiv.). The resulting mixture was stirred for 3 h at 23° C. Once complete transformation was observed by LC/MS analysis the reaction was acidified to pH˜3 with aq. HCl (4 M). The product was extracted with EtOAc (2×10 mL), the combined organic extract was dried over Na₂SO₄ and concentrated to dryness under reduced pressure to yield 1-cyclopropyl-5-ethenyl-2-oxopyridine-3-carboxylic acid.

Step g: To a solution of the product from step e (70 mg, 0.32 mmol, 1.0 equiv.) and the product of step f (100 mg, 0.3165 mmol, 1.0 equiv.) in DMF (3 ml, 0.1M) was added DIPEA (0.15 ml, 0.63 mmol, 2.0 equiv.) followed by HATU (0.24 mg, 0.63 mmol, 2.0 equiv.). The reaction mixture was stirred at room temperature overnight. The resulting mixture was concentrated to dryness, and the crude residue was purified by column chromatography (SiO₂, 0-10% MeOH gradient in dichloromethane) to afford N-[4-[4-cyano-2-(1-methylimidazol-2-yl)phenyl]-6-cyclopropylpyridin-2-yl]-1-cyclopropyl-5-ethenyl-2-oxopyridine-3-carboxamide.

Step h: To a solution the product of step g (0.3 g, 0.6 mmol, 1.0 equiv.) in a THF/water mixture (6 mL, 1:1 v/v) 2,4-lutidine (130 mg, 1.1928 mmol, 2.0 equiv.) was added. The resulting mixture was placed in water bath (˜15° C.). Then sodium periodate (0.51 mg, 2.4 mmol, 4.0 equiv.) and K₂OsO₄·2H₂O (12 mg, 0.03 mmol, 0.05 equiv.) were added sequentially. The reaction was vigorously stirred for 4 h. During the reaction time a formation of white precipitate was observed. Once complete transformation was observed by TLC analysis, the mixture was diluted with water (30 mL) and EtOAc (40 mL). The aqueous phase was extracted with EtOAc (3×30 mL). Combined organic phase was sequentially washed with aq. 1 M HCl (100 mL) and brine (100 mL), dried over Na₂SO₄ and concentrated to dryness to produce N-[4-[4-cyano-2-(1-methylimidazol-2-yl)phenyl]-6-cyclopropylpyridin-2-yl]-1-cyclopropyl-5-formyl-2-oxopyridine-3-carboxamide.

Step i. To the solution of the product from step h (30 mg, 0.06 mmol, 1.0 equiv.) in dichloromethane (3 mL, 0.02 M) was added (2R)-2-methylmorpholine (12 mg, 0.12 mmol, 2.0 equiv.) and DIPEA (0.03 mL, 0.15 mmol, 2.5 equiv.) and the mixture was stirred at 23° C. for 10 mins. NaBH(OAc)₃ (33 mg, 0.15 mmol, 2.5 equiv.) was added and the mixture was stirred at 23° C. overnight. The reaction was quenched with aq. sat. NaHCO₃ (5 mL) and diluted with EtOAc (10 mL). The organic phase was separated, and the aqueous layer was additionally extracted with EtOAc (2×10 mL). The combined organic extract was dried over Na₂SO₄ and concentrated to dryness under reduced pressure. The crude residue was purified by column chromatography (SiO₂, 0-25% MeOH in dichlorormethane) to afford a mixture of two products. Individual compounds were isolated after reversed phase preparative HPLC (C18, 10-90% CH₃CN in water with 0.1% formic acid).

Example 36 (first eluting product): ¹H NMR (400 MHz, Chloroform-d) δ 12.29 (s, 1H), 8.56 (d, J=2.6 Hz, 1H), 8.26 (d, J=1.4 Hz, 1H), 7.93 (d, J=1.7 Hz, 1H), 7.83 (dd, J=8.1, 1.8 Hz, 1H), 7.73 (d, J=8.1 Hz, 1H), 7.57 (s, 1H), 7.15 (d, J=1.2 Hz, 1H), 6.81 (d, J=1.2 Hz, 1H), 6.30 (d, J=1.5 Hz, 1H), 3.85 (d, J=11.7 Hz, 1H), 3.71-3.56 (m, 2H), 3.46 (tt, J=7.6, 4.2 Hz, 1H), 3.33 (s, 2H), 3.00 (s, 3H), 2.64 (d, J=17.8 Hz, 2H), 1.95-1.76 (m, 1H), 1.27 (d, J=7.5 Hz, 2H), 1.14 (d, J=6.3 Hz, 3H), 0.96 (t, J=5.5 Hz, 2H), 0.86 (t, J=6.8 Hz, 6H). ESI MS [M+H]⁺ for C₃₄H₃₆N₇O₃, calcd 590.7, found 590.3.

Example 37 (second eluting product): ¹H NMR (400 MHz, CDCl₃) δ 12.25 (s, 1H), 8.54 (d, J=2.7 Hz, 1H), 8.20-8.07 (m, 1H), 7.91 (d, J=1.7 Hz, 1H), 7.84 (dd, J=8.0, 1.7 Hz, 1H), 7.78 (d, J=2.6 Hz, 1H), 7.73 (d, J=8.1 Hz, 1H), 7.12 (d, J=1.2 Hz, 1H), 6.82 (d, J=1.3 Hz, 1H), 6.39 (d, J=1.5 Hz, 1H), 3.65 (s, 2H), 3.42 (tt, J=7.6, 4.2 Hz, 1H), 3.04 (s, 3H), 1.86 (m, 1H), 1.23 (q, J=6.7, 6.3 Hz, 2H), 1.02-0.94 (m, 2H), 0.88 (d, J=6.5 Hz, 4H). ESI MS [M+H]⁺ for C₂₉H₂₇N₆O₃, calcd 507.3, found 507.1.

### Example 38: N-{4-[4-cyano-2-(1-methyl-2-imidazolyl)phenyl]-6-cyclopropyl-2-pyridyl}-5-({(1S,4S)-5-methyl-2,5-diazabicyclo[2.2.1]hept-2-yl}methyl)-1-cyclopropyl-2-oxo-1,2-dihydronicotinamide

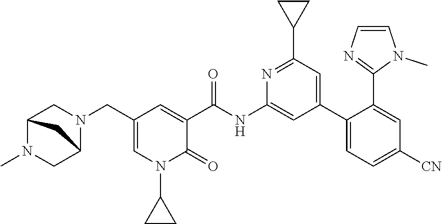

The title compound was prepared in a similar fashion to that described for Example 37, step i starting from N-[4-[4-cyano-2-(1-methylimidazol-2-yl)phenyl]-6-cyclopropylpyridin-2-yl]-1-cyclopropyl-5-formyl-2-oxopyridine-3-carboxamide and (1S,4S)-2-methyl-2,5-diazabicyclo[2.2.1]heptane. ¹H NMR (400 MHz, CDCl₃) δ 12.31 (s, 1H), 8.57 (d, J=2.6 Hz, 1H), 8.25 (d, J=1.4 Hz, 1H), 7.93 (d, J=1.7 Hz, 1H), 7.82 (dd, J=8.0, 1.7 Hz, 1H), 7.73 (d, J=8.1 Hz, 1H), 7.63 (d, J=2.6 Hz, 1H), 7.14 (d, J=1.2 Hz, 1H), 6.81 (d, J=1.2 Hz, 1H), 6.30 (d, J=1.5 Hz, 1H), 3.65 (d, J=13.4 Hz, 1H), 3.59-3.50 (m, 2H), 3.49-3.40 (m, 2H), 3.34 (m, 1H), 3.00 (s, 3H), 2.91 (d, J=10.6 Hz, 1H), 2.73 (m, 2H), 2.57 (s, 3H), 1.91 (s, 2H), 1.81 (tt, J=7.7, 5.3 Hz, 1H), 1.26 (dtd, J=6.6, 4.4, 2.4 Hz, 2H), 0.96 (qd, J=4.2, 1.8 Hz, 2H), 0.86 (ddt, J=7.3, 5.6, 2.6 Hz, 4H). ESI MS [M+H]⁺ for C₃₅H₃₇N₈O₂, calcd 601.3, found 601.1.

### Example 39: N-{4-[4-cyano-2-(1-methyl-2-imidazolyl)phenyl]-6-cyclopropyl-2-pyridyl}-1-cyclopropyl-5-[(1-methylcyclobutylamino)methyl]-2-oxo-1,2-dihydronicotinamide

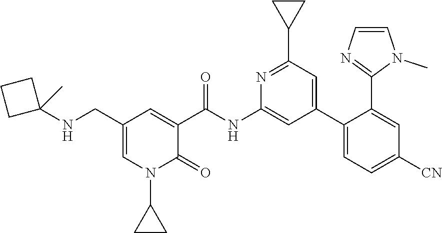 [SMILES: Cn1ccnc1-c1cc(C#N)ccc1-c1cc(NC(=O)c2cc(CNC3(C)CCC3)cn(C3CC3)c2=O)nc(C2CC2)c1]

The title compound was prepared in a similar fashion to that described for example 37, step i from N-[4-[4-cyano-2-(1-methylimidazol-2-yl)phenyl]-6-cyclopropylpyridin-2-yl]-1-cyclopropyl-5-formyl-2-oxopyridine-3-carboxamide and 1-methylcyclobutan-1-amine hydrochloride. ¹H NMR (400 MHz, CDCl₃) δ 12.25 (s, 1H), 8.54 (d, J=2.7 Hz, 1H), 8.15-8.07 (m, 1H), 7.91 (d, J=1.7 Hz, 1H), 7.84 (dd, J=8.0, 1.7 Hz, 1H), 7.78 (d, J=2.6 Hz, 1H), 7.73 (d, J=8.1 Hz, 1H), 7.12 (d, J=1.3 Hz, 1H), 6.82 (d, J=1.3 Hz, 1H), 6.39 (d, J=1.5 Hz, 1H), 3.65 (s, 2H), 3.42 (tt, J=7.6, 4.2 Hz, 1H), 3.04 (s, 3H), 2.13 (d, J=10.3 Hz, 2H), 1.98-1.85 (m, 2H), 1.81 (m, 3H), 1.39 (s, 3H), 1.23 (q, J=6.7, 6.3 Hz, 2H), 1.01-0.95 (m, 2H), 0.88 (d, J=6.5 Hz, 4H). ESI MS [M+H]⁺ for C₃₄H₃₆N₇O₂, calcd 574.3, found 574.1.

### Example 40: N-{4-[4-cyano-2-(1-methyl-2-imidazolyl)phenyl]-6-cyclopropyl-2-pyridyl}-1-cyclopropyl-2-oxo-5-[(2-trifluoromethoxyethylamino)methyl]-1,2-dihydronicotinamide

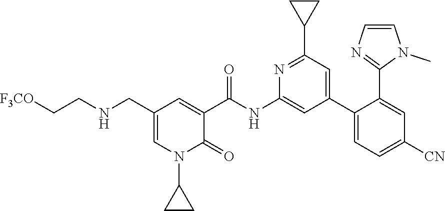 [SMILES: Cn1ccnc1-c1cc(C#N)ccc1-c1cc(NC(=O)c2cc(CNCCOC(F)(F)F)cn(C3CC3)c2=O)nc(C2CC2)c1]

The title compound was prepared in a similar fashion to that described for Example 37, step i from N-[4-[4-cyano-2-(1-methylimidazol-2-yl)phenyl]-6-cyclopropylpyridin-2-yl]-1-cyclopropyl-5-formyl-2-oxopyridine-3-carboxamide and 2-(trifluoromethoxy)ethanamine hydrochloride. ¹H NMR (400 MHz, Chloroform-d) δ 12.25 (s, 1H), 8.54 (d, J=2.7 Hz, 1H), 8.07 (d, J=11.6 Hz, 1H), 7.95-7.81 (m, 2H), 7.73 (d, J=8.0 Hz, 2H), 7.19 (d, J=1.3 Hz, 1H), 6.85 (d, J=1.3 Hz, 1H), 6.42 (s, 1H), 4.13 (t, J=5.1 Hz, 2H), 3.74 (s, 2H), 3.45 (tt, J=7.6, 4.3 Hz, 1H), 3.06 (s, 3H), 3.01 (t, J=5.0 Hz, 2H), 1.84 (p, J=6.5 Hz, 1H), 1.26 (q, J=7.0 Hz, 2H), 0.99 (dd, J=6.3, 4.2 Hz, 2H), 0.89 (d, J=6.5 Hz, 4H). ESI MS [M+H]⁺ for C₃₂H₃₁F₃N₇O₃, calcd 618.3, found 618.1.

### Example 41: N-{4-[4-cyano-2-(1-methyl-2-imidazolyl)phenyl]-6-cyclopropyl-2-pyridyl}-5-{(5-aza-5-spiro[2.4]heptyl)methyl}-1-cyclopropyl-2-oxo-1,2-dihydronicotinamide

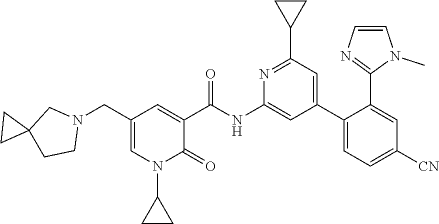 [SMILES: Cn1ccnc1-c1cc(C#N)ccc1-c1cc(NC(=O)c2cc(CN3CCC4(CC4)C3)cn(C3CC3)c2=O)nc(C2CC2)c1]

The title compound was prepared in a similar fashion to that described for example 37, step i from N-[4-[4-cyano-2-(1-methylimidazol-2-yl)phenyl]-6-cyclopropylpyridin-2-yl]-1-cyclopropyl-5-formyl-2-oxopyridine-3-carboxamide and 5-azaspiro[2.4]heptane. ¹H NMR (400 MHz, CDCl₃) δ 12.30 (s, 1H), 8.53 (d, J=2.7 Hz, 1H), 8.26 (d, J=1.5 Hz, 1H), 7.93 (d, J=1.6 Hz, 1H), 7.85-7.82 (m, 1H), 7.75-7.66 (m, 2H), 7.14 (d, J=1.2 Hz, 1H), 6.81 (d, J=1.3 Hz, 1H), 6.29 (d, J=1.5 Hz, 1H), 3.53 (s, 2H), 3.45 (tt, J=7.6, 4.2 Hz, 1H), 3.00 (s, 3H), 2.80 (t, J=6.9 Hz, 2H), 2.55 (s, 2H), 1.90-1.73 (m, 7H), 1.32-1.21 (m, 2H), 1.04-0.94 (m, 2H), 0.86 (ddt, J=7.4, 5.5, 2.5 Hz, 4H). ESI MS [M+H]⁺ for C₃₅H₃₆N₇O₂, calcd 586.3, found 586.1.

### Example 42: N-{4-[4-cyano-2-(1-methyl-2-imidazolyl)phenyl]-6-cyclopropyl-2-pyridyl}-1-cyclopropyl-5-[(2-methoxyethylamino)methyl]-2-oxo-1,2-dihydronicotinamide

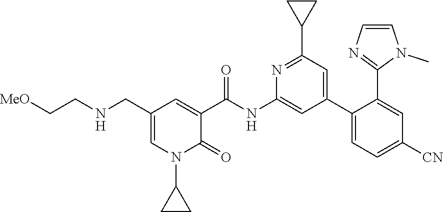 [SMILES: COCCNCc1cc(C(=O)Nc2cc(-c3ccc(C#N)cc3-c3nccn3C)cc(C3CC3)n2)c(=O)n(C2CC2)c1]

The title compound was prepared in a similar fashion to that described for Example 37 using 2-methoxyethylamine in step i. ¹H NMR (400 MHz, CDCl₃) δ 12.27 (s, 1H), 8.54 (d, J=2.7 Hz, 1H), 8.19 (d, J=1.4 Hz, 1H), 7.93 (d, J=1.7 Hz, 1H), 7.83 (dd, J=8.1, 1.7 Hz, 1H), 7.77-7.67 (m, 2H), 7.14 (d, J=1.2 Hz, 1H), 6.81 (d, J=1.2 Hz, 1H), 6.34 (d, J=1.5 Hz, 1H), 3.75 (s, 2H), 3.54 (t, J=5.0 Hz, 2H), 3.45 (tt, J=7.6, 4.3 Hz, 1H), 3.37 (s, 3H), 3.01 (s, 3H), 2.88 (t, J=5.0 Hz, 2H), 1.89-1.72 (m, 1H), 1.24 (q, J=6.9 Hz, 2H), 1.03-0.92 (m, 2H), 0.91-0.82 (m, 4H). ESI MS [M+H]⁺ for C₃₂H₃₄N₇O₃, calcd 564.3, found 564.1.

### Example 43 and 44: N-{4-[4-cyano-2-(1-methyl-2-imidazolyl)phenyl]-6-cyclopropyl-2-pyridyl}-5-{[(S)-3-methyl-1-piperidyl]methyl}-1-cyclopropyl-2-oxo-1,2-dihydronicotinamide and N-{4-[4-carbamoyl-2-(1-methyl-2-imidazolyl)phenyl]-6-cyclopropyl-2-pyridyl}-5-{[(S)-3-methyl-1-piperidyl]methyl}-1-cyclopropyl-2-oxo-1,2-dihydronicotinamide

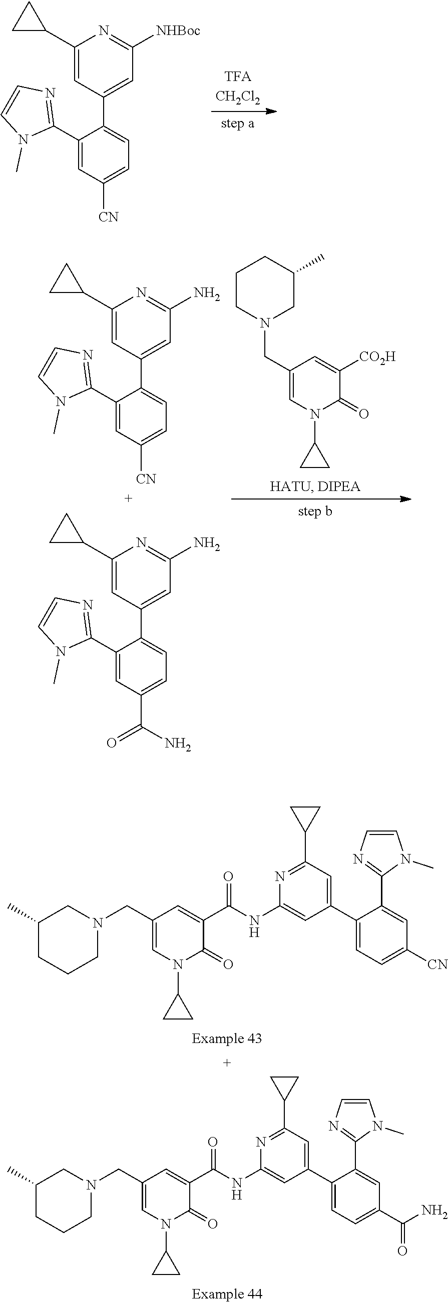

Step a: To a solution of tert-butyl N-[4-[4-cyano-2-(1-methylimidazol-2-yl)phenyl]-6-cyclopropylpyridin-2-yl]carbamate (326 mg, 0.7855 mmol, 1.0 equiv., prepared according to example Examples 37 and 38) in DCM (5 ml, 0.15 M) was add TFA (1 ml, excess). The resulting solution was stirred at rt for 1 h. The solvent was removed, and the mixture was concentrated and the crude was purified by column chromatography (SiO₂, MeOH in DCM, 0 to 10%) to give 4-(2-amino-6-cyclopropylpyridin-4-yl)-3-(1-methylimidazol-2-yl)benzonitrile and 4-(2-amino-6-cyclopropylpyridin-4-yl)-3-(1-methylimidazol-2-yl)benzamide.

Step b: Step b was performed in similar fashion to that described for Example 1, step m. The mixture of two products was separated by reversed phase preparative HPLC (C18, 10-90% gradient CH₃CN in H₂O with 0.1% formic acid).

Example 43: ¹H NMR (400 MHz, CDCl₃) δ 12.30 (s, 1H), 8.51 (d, J=2.6 Hz, 1H), 8.26 (d, J=1.4 Hz, 1H), 7.93 (d, J=1.7 Hz, 1H), 7.82 (dd, J=8.0, 1.7 Hz, 1H), 7.73 (m, 2H), 7.15 (d, J=1.2 Hz, 1H), 6.81 (d, J=1.2 Hz, 1H), 6.30 (d, J=1.4 Hz, 1H), 3.47 (tt, J=7.7, 4.3 Hz, 1H), 3.39 (s, 2H), 3.00 (s, 3H), 2.92-2.74 (m, 2H), 1.99 (m, 2H), 1.81 (tt, J=7.5, 5.3 Hz, 1H), 1.68 (m, 5H), 1.25 (dd, J=7.6, 6.2 Hz, 2H), 0.97 (d, J=4.5 Hz, 2H), 0.86 (dt, J=8.4, 2.8 Hz, 7H). ESI MS [M+H]⁺ for C₃₅H₃₈N₇O₂, calcd 588.7, found 588.1.

Example 44: ¹H NMR (400 MHz, CD₃OD) δ 8.49 (d, J=2.6 Hz, 1H), 8.42-8.28 (m, 1H), 8.06 (dd, J=8.1, 1.9 Hz, 1H), 8.00-7.85 (m, 3H), 7.68 (d, J=8.1 Hz, 1H), 6.95 (dd, J=11.8, 1.4 Hz, 2H), 6.55 (d, J=1.4 Hz, 1H), 4.35 (s, 1H), 3.70 (s, 2H), 3.39 (tt, J=7.8, 4.2 Hz, 1H), 3.14 (s, 3H), 3.10-2.96 (m, 2H), 2.31 (t, J=12.1 Hz, 1H), 2.03 (t, J=11.4 Hz, 1H), 1.82 (tt, J=8.0, 5.0 Hz, 1H), 1.72 (t, J=12.1 Hz, 3H), 1.57 (q, J=14.0, 13.2 Hz, 1H), 1.20-1.08 (m, 2H), 0.93 (td, J=4.8, 4.1, 2.8 Hz, 2H), 0.88-0.73 (m, 7H). ESI MS [M+H]⁺ for C₃₅H₄₀N₇O₃, calcd 606.3, found 606.1.

### Example 45: N-[4-(4-cyano-2-{1-[(²H₃)methyl]-2-imidazolyl}phenyl)-6-cyclopropyl-2-pyridyl]-1-cyclopropyl-5-[(2-methoxyethylamino)methyl]-2-oxo-1,2-dihydronicotinamide

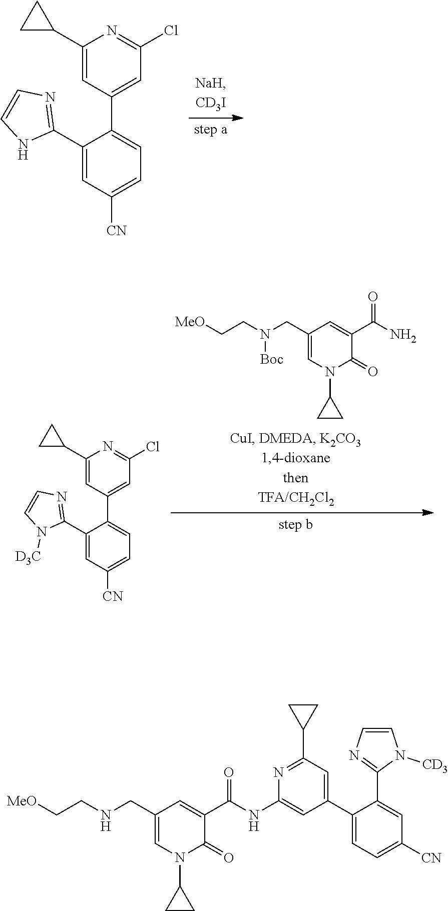 [SMILES: N#Cc1ccc(-c2cc(Cl)nc(C3CC3)c2)c(-c2ncc[nH]2)c1 ; COCCN(Cc1cc(C(N)=O)c(=O)n(C2CC2)c1)C(=O)OC(C)(C)C ; [2H]C([2H])([2H])n1ccnc1-c1cc(C#N)ccc1-c1cc(Cl)nc(C2CC2)c1 ; [2H]C([2H])([2H])n1ccnc1-c1cc(C#N)ccc1-c1cc(NC(=O)c2cc(CNCCOC)cn(C3CC3)c2=O)nc(C2CC2)c1]

Step a: The reaction was performed in a similar fashion to Example 37, step c.

Step b: The reaction was performed in a similar fashion to Example 5, step f.

¹H NMR (400 MHz, CDCl₃) δ 12.18 (s, 1H), 8.56 (d, J=2.6 Hz, 1H), 8.08 (d, J=1.5 Hz, 1H), 7.92 (d, J=1.7 Hz, 1H), 7.88-7.81 (m, 2H), 7.73 (d, J=8.1 Hz, 1H), 7.12 (d, J=1.4 Hz, 1H), 6.81 (d, J=1.3 Hz, 1H), 6.41 (d, J=1.5 Hz, 1H), 3.91 (s, 2H), 3.59 (t, J=5.0 Hz, 2H), 3.42 (tt, J=7.6, 4.2 Hz, 1H), 3.34 (s, 3H), 3.02 (t, J=5.0 Hz, 2H), 1.83 (p, J=6.5 Hz, 1H), 1.21 (dd, J=7.6, 6.1 Hz, 2H), 1.05-0.93 (m, 2H), 0.88 (d, J=6.5 Hz, 4H). ESI MS [M+H]⁺ for C₃₂H₃₁D₃N₇O₃, calcd 567.3, found 567.1.

### Example 46: N-(4-{4-cyano-2-[1-methyl-4-(trifluoromethyl)-2-imidazolyl]phenyl}-6-cyclopropyl-2-pyridyl)-1-cyclopropyl-5-[(2-methoxyethylamino)methyl]-2-oxo-1,2-dihydronicotinamide

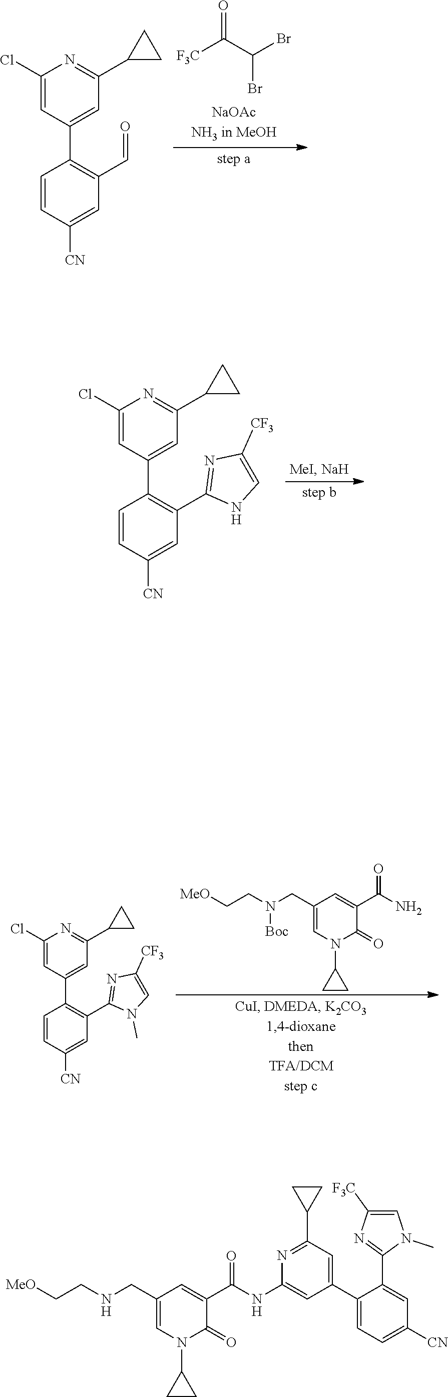

Step a: A mixture of 3,3-dibromo-1,1,1-trifluoropropan-2-one (4.45 g, 2.579 mmol, 1.0 equiv.) and sodium acetate (280 mg, 2.836 mmol, 1.1 equiv.) in H₂O (5 mL) was heated to 100° C. and stirred for 1 h. The resulting mixture was cooled to 0° C. To a solution of 4-(2-chloro-6-cyclopropylpyridin-4-yl)-3-(1H-imidazol-2-yl)benzonitrile (800 mg, 2.836 mmol, 1.1 equiv.) and ammonia solution in methanol (5 ml, 7M) was added the above solution at rt. The mixture was stirred for 4 h. The solution was concentrated, diluting with EtOAc and water. The organic phase was separated, and the aqueous layer was extracted with EtOAc. The crude residue was purified by column chromatography (SiO₂, EtOAc in hexanes, 0 to 20%) to give 4-bromo-3-[4-(trifluoromethyl)-1H-imidazol-2-yl]benzonitrile.

Step b: To a solution of the product of step a (214 mg, 0.5504 mmol, 1.0 equiv.) in THF (5.0 mL, 0.1 M) was added NaH (45 mg, 1.1008 mmol, 2.5 equiv., 60 wt % in oil) at 0° C. The resulting mixture was stirred for 10 min at 0° C. before MeI (0.06 ml, 0.8256 mmol, 1.5 equiv.) was added. The reaction was allowed to warm to room temperature and stirred for 3 h. The reaction was quenched with sat. aq. NH₄Cl solution (1 mL) and diluted with EtOAc (10 mL). The organic phase was separated, and the aqueous layer was extracted with EtOAc (10 mL). The combined organic phase was dried over Na₂SO₄, concentrated to dryness, and the crude residue was purified by column chromatography (SiO₂, 0-10% MeOH gradient in dichloromethane) to give 4-(2-chloro-6-cyclopropylpyridin-4-yl)-3-[1-methyl-4-(trifluoromethyl)imidazol-2-yl]benzonitrile.

Step c: The reaction was performed in a similar fashion to step f of Example 5. ¹H NMR (400 MHz, CDCl₃) δ 12.31 (s, 1H), 8.53 (d, J=2.6 Hz, 1H), 8.22 (d, J=1.4 Hz, 1H), 7.95 (d, J=1.7 Hz, 1H), 7.87 (dd, J=8.0, 1.7 Hz, 1H), 7.79-7.61 (m, 2H), 7.15 (d, J=1.3 Hz, 1H), 6.38 (d, J=1.4 Hz, 1H), 3.71 (s, 2H), 3.60-3.50 (m, 2H), 3.45 (dq, J=7.7, 4.3, 3.8 Hz, 1H), 3.37 (s, 3H), 3.05 (s, 3H), 2.90-2.78 (m, 2H), 1.90-1.67 (m, 2H), 1.38-1.19 (m, 2H), 0.98 (dd, J=6.4, 4.3 Hz, 2H), 0.95-0.79 (m, 4H). ESI MS [M+H]⁺ for C₃₃H₃₃F₃N₇O₂, calcd 632.3, found 632.1.

### Example 47: N-(4-{4-cyano-2-[1-methyl-4-(trifluoromethyl)-2-imidazolyl]phenyl}-6-cyclopropyl-2-pyridyl)-5-{[(S)-3-methyl-1-piperidyl]methyl}-1-cyclopropyl-2-oxo-1,2-dihydronicotinamide

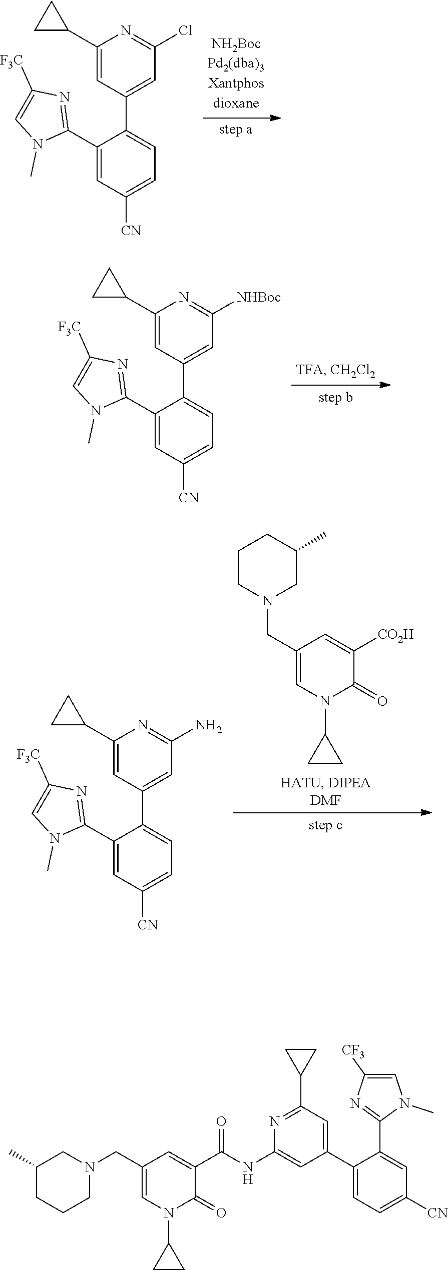

The title compound was prepared via steps a-c in a similar fashion described to Example 7, steps f-h, starting from 4-(2-chloro-6-cyclopropylpyridin-4-yl)-3-[1-methyl-4-(trifluoromethyl)imidazol-2-yl]benzonitrile (prepared according to Example 47). ¹H NMR (400 MHz, CDCl₃) δ 12.25 (d, J=6.1 Hz, 1H), 8.71-8.38 (m, 1H), 8.17 (dd, J=4.6, 1.4 Hz, 1H), 8.08-7.86 (m, 2H), 7.77 (dd, J=17.0, 8.1 Hz, 2H), 7.16-6.95 (m, 1H), 6.39 (dd, J=28.0, 1.5 Hz, 1H), 3.55 (s, 2H), 3.46 (tt, J=7.7, 4.3 Hz, 1H), 3.07 (d, J=29.8 Hz, 3H), 2.95 (s, 1H), 2.15 (d, J=10.4 Hz, 1H), 1.90-1.55 (m, 8H), 1.26 (t, J=6.9 Hz, 2H), 1.00 (d, J=9.2 Hz, 2H), 0.96-0.72 (m, 6H). ESI MS [M+H]⁺ for C₃₆H₃₇F₃N₇O₂, calcd 656.3, found 656.1.

### Example 48: N-{4-[4-cyano-2-(4-cyano-1-methyl-2-imidazolyl)phenyl]-6-cyclopropyl-2-pyridyl}-1-cyclopropyl-5-[(2-methoxyethylamino)methyl]-2-oxo-1,2-dihydronicotinamide

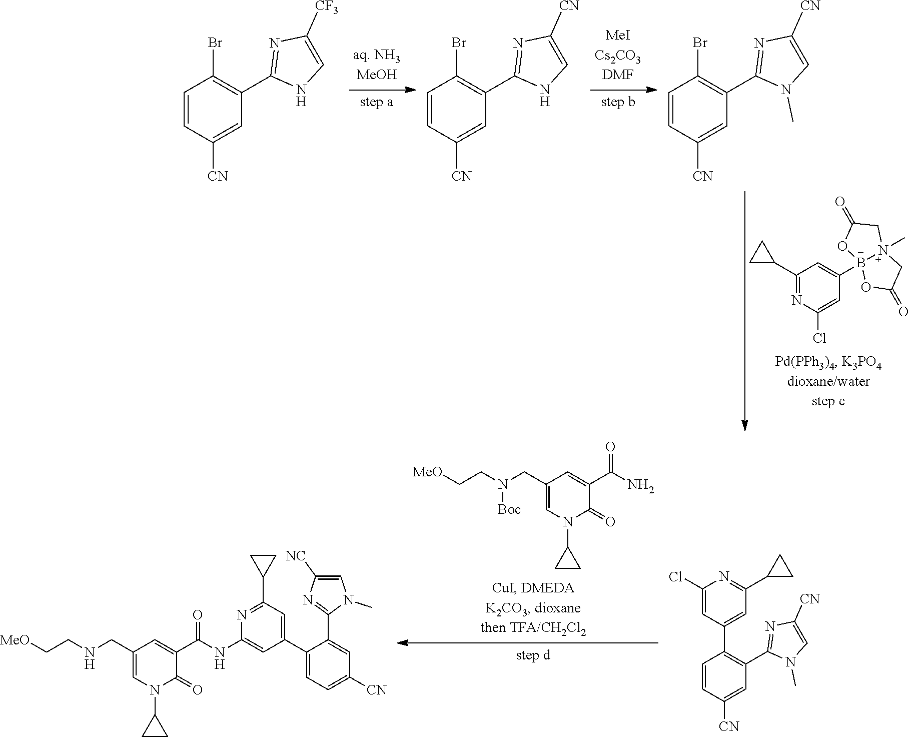

Step a: 4-Bromo-3-[4-(trifluoromethyl)-1H-imidazol-2-yl]benzonitrile (500 mg, 1.6 mmol, 1.0 equiv., obtained in a similar fashion to Example 47, step a, starting from 4-bromo-3-formylbenzonitrile) was suspended in ammonium hydroxide solution (5 mL, excess, 28% wt in H₂O) and placed in a sealed tube. The resulting mixture was heated at 60° C. for overnight. After cooling to room temperature, the solution was concentrated to dryness under reduced pressure, diluted with EtOAc (30 mL) and water (30 mL). The organic phase was separated, and the aqueous layer was extracted with EtOAc (20 mL). The combined organic extract was dried over Na₂SO₄ and concentrated to dryness under reduced pressure. The crude residue was purified by column chromatography (SiO₂, 0-20% EtOAc in hexanes) to yield 2-(2-bromo-5-cyanophenyl)-1H-imidazole-4-carbonitrile.

Step b: To a solution of the product of step a (820 mg, 3.0 mmol, 1.0 equiv.) in DMF (15.0 mL, 0.2 M) was added Cs₂CO₃ (2 g, 6.0 mmol, 2.0 equiv.) at 0° C. The resulting mixture was stirred for 10 min at 0° C. before MeI (0.3 ml, 4.5 mmol, 1.5 equiv.) was added. The reaction was allowed to warm to room temperature and stirred for 1 h. The reaction was quenched with sat. aq. NH₄Cl solution (1 mL) and diluted with EtOAc (10 mL), the organic phase was separated, and the aqueous layer was extracted with EtOAc (10 mL). The combined organic phase was dried over Na₂SO₄, concentrated to dryness, and the crude residue was purified by column chromatography (SiO₂, 0-10% MeOH gradient in dichloromethane) to give 2-(2-bromo-5-cyanophenyl)-1-methylimidazole-4-carbonitrile.

Step c: To a solution of the product of step b (310 mg, 1.080 mmol, 1.0 equiv.), 1-(2-chloro-6-cyclopropylpyridin-4-yl)-5-methyl-2,8-dioxa-5-azonia-1-boranuidabicyclo[3.3.0]octane-3,7-dione (332 mg, 1.080 mmol, 1.0 equiv., prepared according to protocol described for Example 15) and K₃PO₄ (0.69 g, 3.24 mmol, 3.0 equiv.) in dioxane water mixture (6 mL, 5:1 v/v, 0.16 M) was added Pd(PPh₃)₄ (124 mg, 0.108 mmol, 0.1 equiv.). The mixture was sparged with nitrogen for 10 min and stirred at 90° C. for 1 h. The resulting mixture was cooled to room temperature and diluted with sat. aq. NH₄Cl solution (50 mL), water (50 mL) and EtOAc (100 mL). The organic phase was separated, and the aqueous phase was additionally extracted with EtOAc (2×30 mL). The combined organic phase was dried over Na₂SO₄ and concentrated to dryness under reduced pressure. The crude product was purified by column chromatography (SiO₂, 0-60% EtOAc gradient in hexanes) to afford 2-[2-(2-chloro-6-cyclopropylpyridin-4-yl)-5-cyanophenyl]-1-methylimidazole-4-carbonitrile.

Step d: The reaction was performed in a similar fashion to Example 5, step f. ¹H NMR (400 MHz, CDCl₃) δ 12.28 (s, 1H), 8.53 (d, J=2.7 Hz, 1H), 8.10 (d, J=1.5 Hz, 1H), 7.89 (dt, J=4.4, 2.0 Hz, 2H), 7.75 (d, J=8.5 Hz, 1H), 7.70 (d, J=2.7 Hz, 1H), 7.37 (s, 1H), 6.49 (d, J=1.5 Hz, 1H), 3.70 (s, 2H), 3.58-3.50 (m, 2H), 3.45 (tt, J=7.6, 4.2 Hz, 1H), 3.37 (s, 3H), 3.12 (s, 3H), 2.91-2.78 (m, 2H), 1.85 (tt, J=7.5, 5.8 Hz, 1H), 1.25 (dd, J=7.8, 6.2 Hz, 2H), 1.02-0.89 (m, 6H). ESI MS [M+H]⁺ for C₃₃H₃₃N₈O₃, calcd 589.3, found 589.1.

### Example 49: N-{4-[4-cyano-2-(4-cyano-1-methyl-2-imidazolyl)phenyl]-6-cyclopropyl-2-pyridyl}-1-cyclopropyl-5-[(1-methylcyclobutylamino)methyl]-2-oxo-1,2-dihydronicotinamide

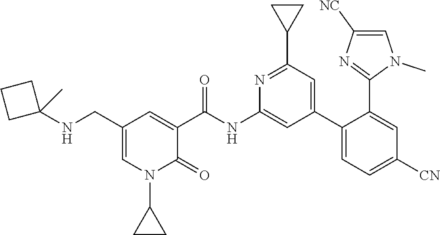 [SMILES: Cn1cc(C#N)nc1-c1cc(C#N)ccc1-c1cc(NC(=O)c2cc(CNC3(C)CCC3)cn(C3CC3)c2=O)nc(C2CC2)c1]

The title compound was prepared in a similar fashion to that described for Example 49, using 1-cyclopropyl-5-[[(1-methylcyclobutyl)amino]methyl]-2-oxopyridine-3-carboxamide in step d. ¹H NMR (400 MHz, CDCl₃) δ 12.26 (s, 1H), 8.55 (d, J=2.7 Hz, 1H), 8.10 (d, J=1.5 Hz, 1H), 7.90 (dd, J=4.3, 2.5 Hz, 2H), 7.81-7.64 (m, 2H), 7.37 (s, 1H), 6.51 (d, J=1.5 Hz, 1H), 3.59 (s, 2H), 3.44 (tt, J=7.6, 4.2 Hz, 1H), 3.11 (s, 3H), 2.02 (q, J=9.5, 9.1 Hz, 2H), 1.94-1.68 (m, 5H), 1.34 (s, 3H), 1.28-1.19 (m, 2H), 1.07-0.86 (m, 6H). ESI MS [M+H]⁺ for C₃₅H₃₅N₈O₂, calcd 599.3, found 600.1.

### Example 50 and 51: N-{4-[4-cyano-2-(5-methyl-1,3-oxazol-4-yl)phenyl]-6-cyclopropyl-2-pyridyl}-1-cyclopropyl-5-[(2-methoxyethylamino)methyl]-2-oxo-1,2-dihydronicotinamide and methyl 4-(6-cyclopropyl-2-{1-cyclopropyl-5-[(2-methoxyethylamino)methyl]-2-oxo-1,2-dihydronicotinoylamino}-4-pyridyl)-3-(5-methyl-1,3-oxazol-4-yl)benzoate

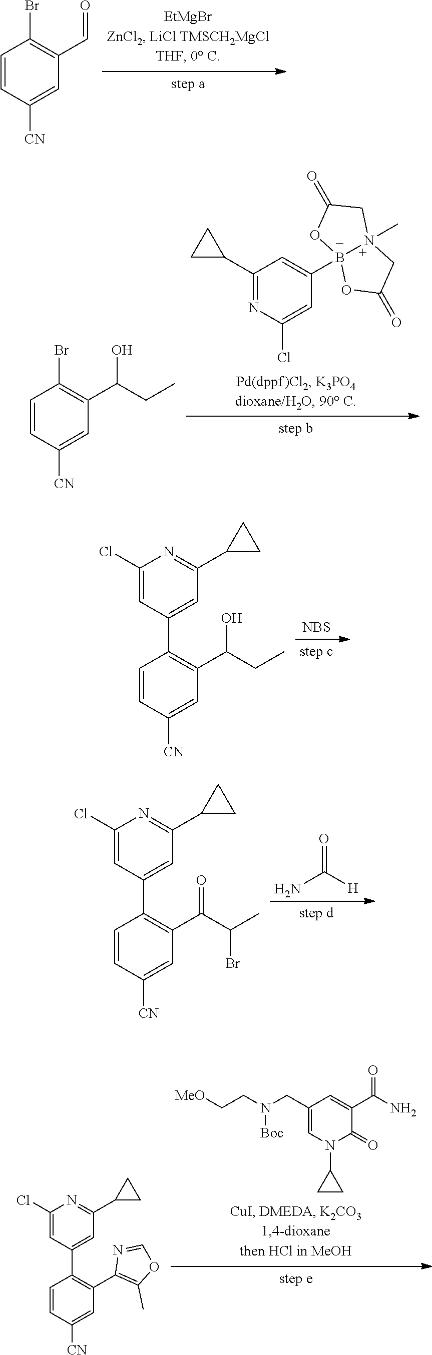

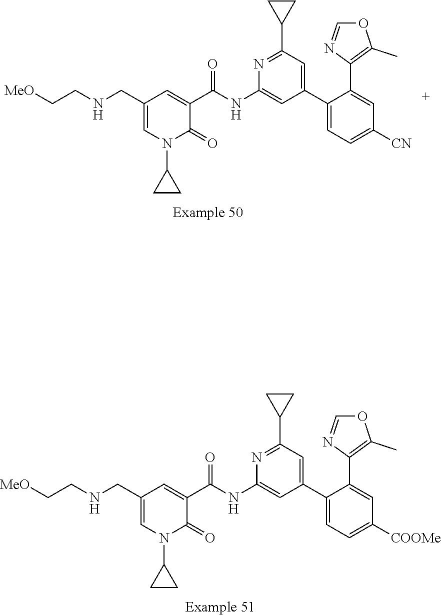 [SMILES: COCCNCc1cc(C(=O)Nc2cc(-c3ccc(C#N)cc3-c3ncoc3C)cc(C3CC3)n2)c(=O)n(C2CC2)c1 ; COCCNCc1cc(C(=O)Nc2cc(-c3ccc(C(=O)OC)cc3-c3ncoc3C)cc(C3CC3)n2)c(=O)n(C2CC2)c1]

Step a: To a Pyrex Schlenk tube was added ZnCl₂ (0.33 g, 2.38 mmol, 0.1 equiv.) and LiCl (1.13 g, 26.2 mmol, 1.1 equiv.), vacuum was applied and the solid mixture was dried under vacuum for 10 min with a heat gun. Then the flask was cooled under nitrogen to room temperature. (Trimethylsilyl)methylmagnesium chloride (4.8 ml, 4.76 mmol, 1M in Et₂O, 0.2 equiv.) was added at room temperature. This mixture was stirred for 15 min. Then EtMgBr (3 M in THF, 8.8 mL, 26.2 mmol) was added, and the solution was stirred at that temperature for 45 min. Then, the solution was cooled at 0° C., and 4-bromo-3-formylbenzonitrile (5.0 g, 23.8 mmol, 1.0 equiv.) in THF (100 ml, 0.2 M) was added over 1 h via a syringe pump. The mixture was stirred at 0° C. for 2 h, then quenched with aq. sat. NH₄Cl (10 mL) and diluted with EtOAc (100 mL) and water (50 mL). The organic phase was separated, the aqueous phase was additionally extracted with EtOAc (2×40 mL). The combined organic extract was washed with brine (100 mL), dried over Na₂SO₄ and concentrated to dryness under reduced pressure. The crude product was purified by column chromatography (SO2, 0-20% EtOAc gradient in hexanes) to afford 4-bromo-3-(1-hydroxypropyl)benzonitrile.

Step b: To a solution of the product of step a (1.1 g, 4.622 mmol, 1.0 equiv.), 1-(2-chloro-6-cyclopropylpyridin-4-yl)-5-methyl-2,8-dioxa-5-azonia-1-boranuidabicyclo[3.3.0]octane-3,7-dione (1.43 g, 4.622 mmol, 1.0 equiv., prepared according to protocol described for Example 15) and K₃PO₄ (3 g, 13.866 mmol, 3.0 equiv.) in dioxane water mixture (35 mL, 6:1 v/v, 0.15 M) was added Pd(dppf)Cl₂ (340 mg, 0.4622 mmol, 0.1 equiv.). The mixture was sparged with nitrogen for 10 min and stirred at 90° C. for 1 h. The resulting mixture was cooled to room temperature and diluted with sat. aq. NH₄Cl solution (100 mL), water (100 mL) and EtOAc (200 mL). The organic phase was separated, and the aqueous phase was additionally extracted with EtOAc (2×100 mL). The combined organic phase was dried over Na₂SO₄ and concentrated to dryness under reduced pressure. The crude product was purified by column chromatography (SiO₂, 0-60% EtOAc gradient in hexanes) to give 4-(2-chloro-6-cyclopropylpyridin-4-yl)-3-(1-hydroxypropyl)benzonitrile.

Step c: To a solution of the product from step b (0.62 g, 2.0 mmol, 1.0 equiv.) in dioxane (20 ml) was added NBS (0.9 g, 5.0 mmol, 2.5 equiv.). The resulting mixture was heated at 70° C. for 6 h. After cooling to room temperature, the solution was concentrated to dryness under reduced pressure, diluted with EtOAc (50 mL) and water (50 mL). The organic phase was separated, and the aqueous layer was extracted with EtOAc (30 mL). The crude residue was purified by column chromatography (SiO₂, 0-20% EtOAc gradient in hexanes) to afford 3-(2-bromopropanoyl)-4-(2-chloro-6-cyclopropylpyridin-4-yl)benzonitrile.

Step d: To a solution of the product from step c (0.5 g, 1.3 mmol, 1.0 equiv.) in DMF (20 ml) was added formamide (2 mL, excess). The resulting mixture was heated at 160° C. for 6 h. The resulting solution was cooled to room temperature and diluted with water (70 mL) and EtOAc (70 mL). The organic phase was separated, and the aqueous layer was extracted with EtOAc (30 mL). The combined organic extract was dried over Na₂SO₄ and concentrated to dryness under reduced pressure. The crude residue was purified by column chromatography (SiO₂, 0-30% EtOAc gradient in hexanes) to yield 4-(2-chloro-6-cyclopropylpyridin-4-yl)-3-(5-methyl-1,3-oxazol-4-yl)benzonitrile.

Step e: The reaction was performed in a similar fashion to step f Example 5. The products were separated by reversed phase preparative HPLC (10-90% CH₃CN in water with 0.1% formic acid).

Example 50: (first eluting product)¹H NMR (400 MHz, CDCl₃) δ 12.18 (s, 1H), 8.52 (d, J=2.7 Hz, 1H), 8.06 (d, J=1.4 Hz, 1H), 7.92-7.78 (m, 2H), 7.73 (dd, J=8.0, 1.9 Hz, 2H), 7.61 (d, J=8.0 Hz, 1H), 6.65 (d, J=1.5 Hz, 1H), 3.75 (s, 2H), 3.60-3.50 (m, 2H), 3.45 (tt, J=7.6, 4.2 Hz, 1H), 3.36 (s, 3H), 2.87 (t, J=5.0 Hz, 2H), 1.87 (s, 4H), 1.24 (d, J=7.1 Hz, 2H), 1.05-0.86 (m, 6H). ESI MS [M+H]⁺ for C₃₂H₃₃N₆O₄, calcd 565.3, found 565.1.

Example 51: (second eluting product)¹H NMR (400 MHz, CDCl₃) δ 12.15 (s, 1H), 8.53 (d, J=2.7 Hz, 1H), 8.21 (d, J=1.8 Hz, 1H), 8.15-8.05 (m, 2H), 7.78 (s, 1H), 7.73 (d, J=2.7 Hz, 1H), 7.58 (d, J=8.1 Hz, 1H), 6.66 (d, J=1.4 Hz, 1H), 3.95 (s, 3H), 3.74 (s, 2H), 3.53 (t, J=5.0 Hz, 2H), 3.49 (s, 2H), 3.45 (dt, J=7.4, 3.3 Hz, 1H), 3.36 (s, 3H), 2.87 (t, J=5.0 Hz, 2H), 1.89 (s, 4H), 1.25 (dd, J=8.0, 6.1 Hz, 2H), 1.06-0.84 (m, 6H). ESI MS [M+H]⁺ for C₃₃H₃₆N₅O₆, calcd 598.3, found 598.1.

### Example 52: N-{4-[4-cyano-2-(4-methyl-1,3-oxazol-5-yl)phenyl]-6-cyclopropyl-2-pyridyl}-1-cyclopropyl-5-[(2-methoxyethylamino)methyl]-2-oxo-1,2-dihydronicotinamide

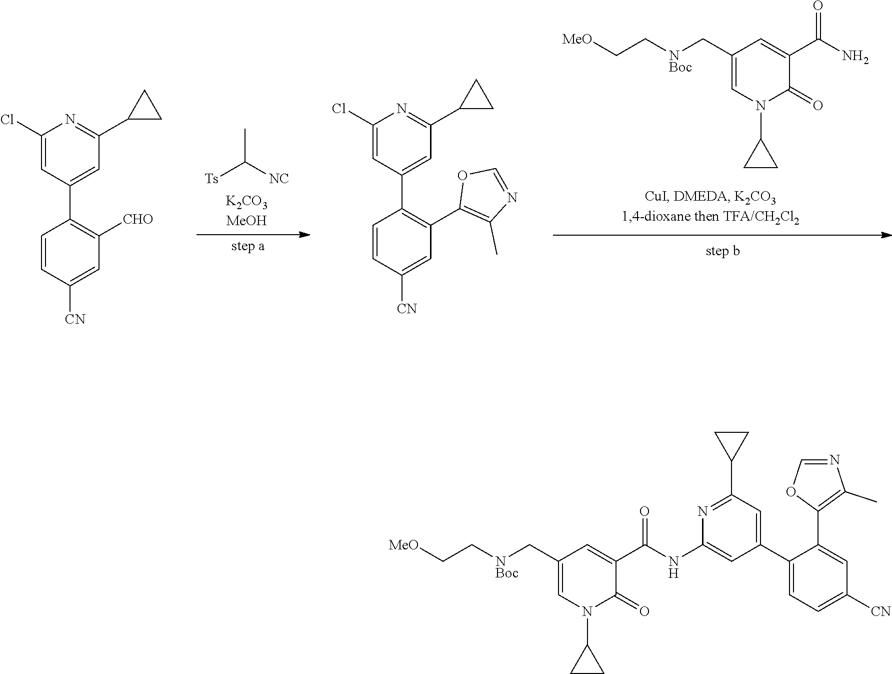

Step a: To a solution of 4-(2-chloro-6-cyclopropylpyridin-4-yl)-3-(1-methylimidazol-2-yl)benzonitrile (450 mg, 1.60 mmol, 1.0 equiv., prepared according to protocol described for Examples 37 and 38) in MeOH (5 ml) was added 1-(1-isocyanoethylsulfonyl)-4-methylbenzene (0.34 g, 1.6 mmol, 1.0 equiv.) and K₂CO₃ (0.27 g, 1.9 mmol, 1.2 equiv.). The resulting mixture was heated to 80° C. for 3 h. After cooling to room temperature, the solution was concentrated and diluted with EtOAc (30 mL) and water (20 mL). The organic phase was separated, and the aqueous layer was extracted with EtOAc (2×15 mL). The crude residue was purified by column chromatography (SiO₂, 0-60% EtOAc gradient in hexanes) to afford 4-(2-chloro-6-cyclopropylpyridin-4-yl)-3-(4-methyl-1,3-oxazol-5-yl)benzonitrile.

Step b: The reaction was performed in a similar fashion to step f Example 5. ¹H NMR (400 MHz, CDCl₃) δ 12.22 (s, 1H), 8.51 (d, J=2.6 Hz, 1H), 8.06 (d, J=1.4 Hz, 1H), 7.82-7.74 (m, 3H), 7.70-7.57 (m, 2H), 6.60 (d, J=1.4 Hz, 1H), 3.69 (s, 2H), 3.59-3.47 (m, 2H), 3.45 (tt, J=7.6, 4.2 Hz, 1H), 3.37 (s, 3H), 2.89-2.72 (m, 2H), 1.94 (s, 3H), 1.91-1.82 (m, 1H), 1.32-1.20 (m, 2H), 1.06-0.85 (m, 6H). ESI MS [M+H]⁺ for C₃₂H₃₃N₆O₄, calcd 565.3, found 565.1.

### Example 53: N-{4-[4-cyano-2-(5-methyl-1,3-oxazol-4-yl)phenyl]-6-cyclopropyl-2-pyridyl}-1-cyclopropyl-5-[(1-methylcyclobutylamino)methyl]-2-oxo-1,2-dihydronicotinamide

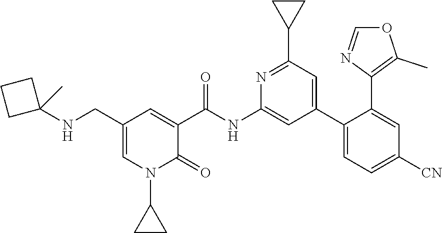 [SMILES: Cc1ocnc1-c1cc(C#N)ccc1-c1cc(NC(=O)c2cc(CNC3(C)CCC3)cn(C3CC3)c2=O)nc(C2CC2)c1]

The title compound was prepared in a similar fashion to that described for Example 53, using 1-cyclopropyl-5-[[(1-methylcyclobutyl)amino]methyl]-2-oxopyridine-3-carboxamide in step b. ¹H NMR (400 MHz, CDCl₃) δ 12.21 (s, 1H), 8.52 (d, J=2.7 Hz, 1H), 8.09 (d, J=1.4 Hz, 1H), 7.86 (d, J=1.7 Hz, 1H), 7.79 (s, 1H), 7.76-7.64 (m, 2H), 7.61 (d, J=8.0 Hz, 1H), 6.63 (d, J=1.4 Hz, 1H), 3.57 (s, 2H), 3.44 (tt, J=7.7, 4.1 Hz, 1H), 2.09-1.93 (m, 2H), 1.87 (s, 3H), 1.76 (m, 4H), 1.33 (s, 3H), 1.25 (m, 2H), 0.96 (h, J=3.8, 3.1 Hz, 6H), 0.90 (dt, J=8.2, 2.9 Hz, 2H). ESI MS [M+H]⁺ for C₃₄H₃₅N₆O₃, calcd 575.3, found 575.1.

### Example 54: N-{4-[4-cyano-2-(2,4-dimethyl-5-imidazolyl)phenyl]-6-cyclopropyl-2-pyridyl}-1-cyclopropyl-5-[(2-methoxyethylamino)methyl]-2-oxo-1,2-dihydronicotinamide

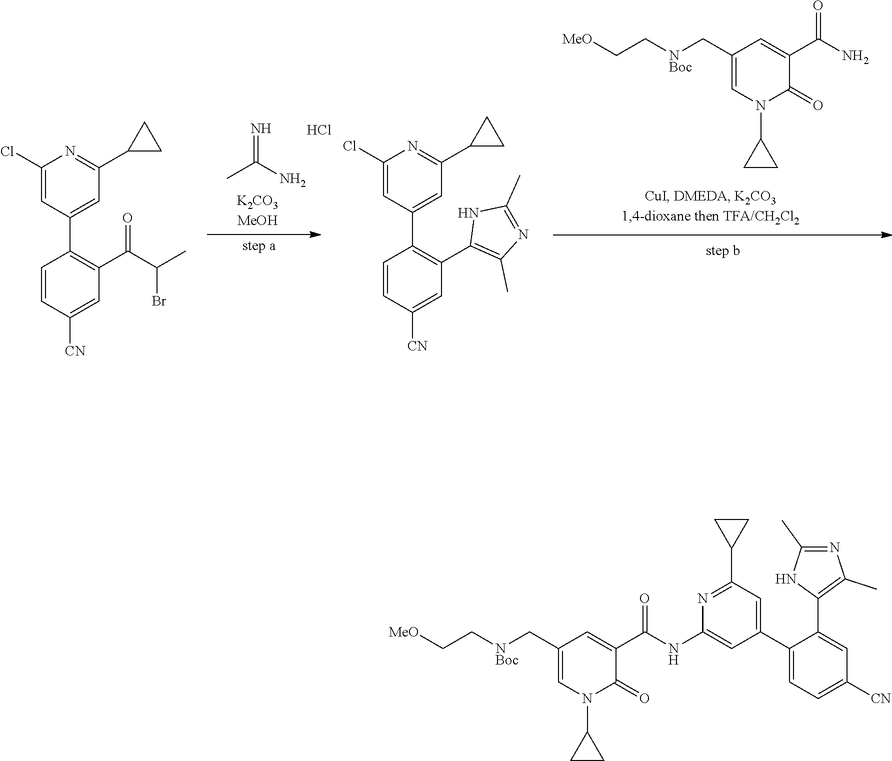

Step a: To a solution of 3-(2-bromopropanoyl)-4-(2-chloro-6-cyclopropylpyridin-4-yl)benzonitrile (0.25 g, 0.65 mmol, 1.0 equiv., prepared according to Examples 51 and 52) in CH₃CN (3 ml) was added acetamidine hydrochloride (0.13 g, 1.3 mmol, 2.0 equiv.) and K₂CO₃ (0.27 g, 1.95 mmol, 3.0 equiv.). The resulting mixture was heated at 80° C. for 15 h, then cooled to room temperature and concentrated to dryness under reduced pressure. The dry residue was dissolved in EtOAc (15 mL), washed with water (15 mL), dried over Na₂SO₄ and concentrated to dryness under reduced pressure. The crude residue was purified by column chromatography (SiO₂, 0-60% EtOAc in hexanes) to afford 4-(2-chloro-6-cyclopropylpyridin-4-yl)-3-(2,4-dimethyl-1H-imidazol-5-yl)benzonitrile.

Step b: The title compound was prepared in a similar fashion to that described for Example 53. ¹H NMR (400 MHz, CDCl₃) δ 12.15 (s, 1H), 8.56 (d, J=2.6 Hz, 1H), 7.95 (s, 1H), 7.74 (dd, J=4.5, 2.2 Hz, 2H), 7.67 (d, J=1.7 Hz, 1H), 7.56 (d, J=8.0 Hz, 1H), 6.60 (s, 1H), 3.75 (s, 2H), 3.55 (t, J=5.0 Hz, 2H), 3.44 (tt, J=7.6, 4.2 Hz, 1H), 3.35 (s, 3H), 2.89 (t, J=5.0 Hz, 2H), 2.26 (s, 3H), 1.97-1.78 (m, 4H), 1.23 (q, J=6.8 Hz, 2H), 1.07-0.81 (m, 6H). ESI MS [M+H]⁺ for C₃₃H₃₆N₇O₃, calcd 578.3, found 578.1.

### Example 55: N-{4-[4-cyano-2-(4-methyl-1,3-oxazol-5-yl)phenyl]-6-cyclopropyl-2-pyridyl}-1-cyclopropyl-5-[(2-methoxyethylamino)methyl]-2-oxo-1,2-dihydronicotinamide

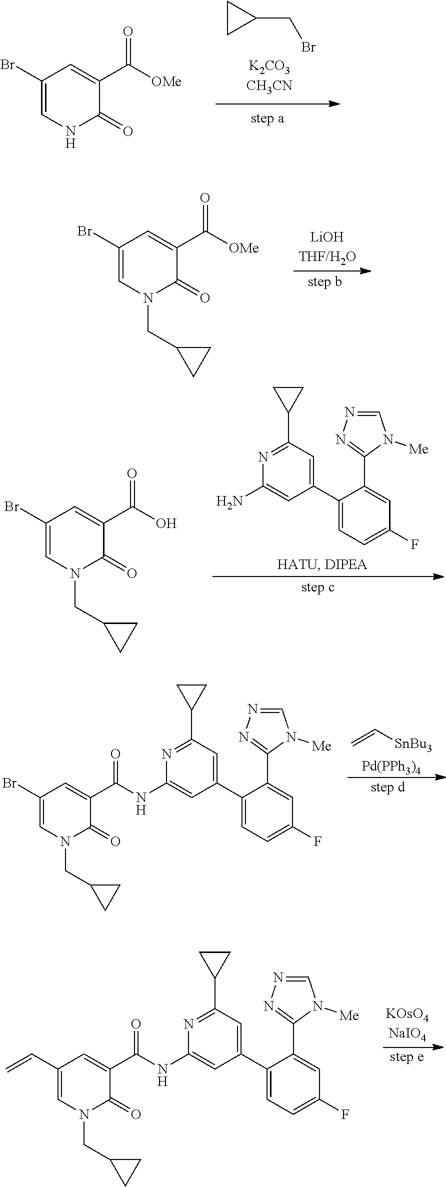

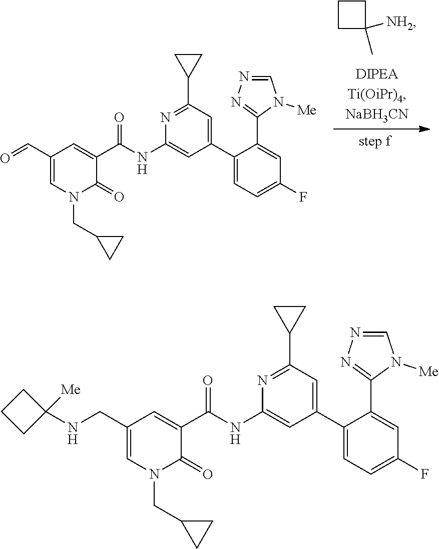

Step a: To a solution of methyl 5-bromo-2-oxo-1H-pyridine-3-carboxylate (2.3 g, 10.0 mmol, 1.0 equiv.) in CH₃CN (30 ml) was added bromomethylcyclopropane (2.0 g, 15.0 mmol, 1.5 equiv.) and K₂CO₃ (3.5 g, 25 mmol, 2.5 equiv.). The resulting mixture was stirred at room temperature for 24 h. Then it was diluted with EtOAc (70 mL) and water (70 mL). The organic phase was separated, and the aqueous layer was extracted with EtOAc (2×25 mL). The combined organic extract was washed with water (100 mL), dried over Na₂SO₄ and concentrated to dryness under reduced pressure. The crude residue was purified by column chromatography (SiO₂, 0-80% EtOAc gradient in hexanes) to afford methyl 5-bromo-1-(cyclopropylmethyl)-2-oxopyridine-3-carboxylate.

Steps b-f were performed in a similar fashion to steps described in example 1. ¹H NMR (400 MHz, CDCl₃) δ 12.37 (s, 1H), 8.56 (d, J=2.7 Hz, 1H), 8.14 (d, J=1.5 Hz, 1H), 8.06 (s, 1H), 7.73 (d, J=2.6 Hz, 1H), 7.64 (dd, J=8.5, 5.4 Hz, 1H), 7.48-7.29 (m, 2H), 6.47 (d, J=1.5 Hz, 1H), 4.67 (s, 1H), 3.94 (d, J=7.2 Hz, 2H), 3.59 (s, 2H), 3.09 (s, 3H), 2.28 (dt, J=12.3, 9.3 Hz, 1H), 2.01-1.92 (m, 2H), 1.92-1.71 (m, 4H), 1.32 (s, 4H), 0.88 (dd, J=6.4, 3.1 Hz, 4H), 0.76-0.62 (m, 2H), 0.44 (q, J=5.2 Hz, 2H). ESI MS [M+H]⁺ for C₃₃H₃₇FN₇O₂, calcd 582.3, found 582.1.

### Example 56: N-[6-cyclopropyl-4-[4-fluoro-2-(4-methyl-1,2,4-triazol-3-yl)phenyl]pyridin-2-yl]-1-(cyclopropylmethyl)-5-[[(3S)-3-methylpiperidin-1-yl]methyl]-2-oxopyridine-3-carboxamide

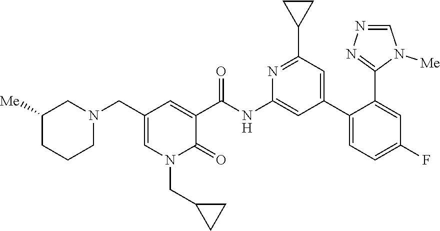 [SMILES: C[C@H]1CCCN(Cc2cc(C(=O)Nc3cc(-c4ccc(F)cc4-c4nncn4C)cc(C4CC4)n3)c(=O)n(CC3CC3)c2)C1]

The title compound was prepared in a similar fashion to that described for Example 56 using (S)-3-methylpiperidine for the reductive amination step and bromomethylcyclopropane for N-alkylation. ¹H NMR (400 MHz, DMSO-d₆) δ 12.38 (s, 1H), 8.46 (s, 1H), 8.35 (d, J=2.6 Hz, 1H), 8.05 (d, J=2.6 Hz, 1H), 7.73 (d, J=1.4 Hz, 1H), 7.68 (dd, J=8.6, 5.7 Hz, 1H), 7.55 (ddd, J=18.7, 9.0, 2.8 Hz, 2H), 6.65 (d, J=1.5 Hz, 1H), 3.91 (d, J=7.3 Hz, 2H), 3.29-3.26 (m, 3H), 2.75-2.60 (m, 3H), 2.33-2.25 (m, 1H), 1.93 (td, J=8.3, 4.4 Hz, 1H), 1.82 (t, J=11.2 Hz, 1H), 1.68-1.50 (m, 4H), 1.49-1.33 (m, 1H), 1.32-1.19 (m, 1H), 0.94-0.83 (m, 2H), 0.83-0.71 (m, 6H), 0.50-0.34 (m, 4H). ESI MS [M+H]⁺ for C₃₄H₃₉FN₇O₂, calcd 596.3, found 596.3.

### Example 57: N-[6-cyclopropyl-4-[4-fluoro-2-(4-methyl-1,2,4-triazol-3-yl)phenyl]pyridin-2-yl]-5-[[(3S)-3-methylpiperidin-1-yl]methyl]-2-oxo-1-propan-2-ylpyridine-3-carboxamide

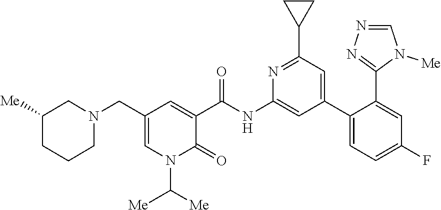 [SMILES: CC(C)n1cc(CN2CCC[C@H](C)C2)cc(C(=O)Nc2cc(-c3ccc(F)cc3-c3nncn3C)cc(C3CC3)n2)c1=O]

The title compound was prepared in a similar fashion to that described for Example 56 using (S)-3-methylpiperidine for the reductive amination step and 2-iodopropane for N-alkylation. ¹H NMR (400 MHz, DMSO-d₆) δ 12.42 (s, 1H), 8.46 (s, 1H), 8.35 (s, 1H), 8.03 (s, 1H), 7.74-7.64 (m, 2H), 7.59-7.47 (m, 2H), 6.67 (d, J=1.4 Hz, 1H), 5.19 (p, J=6.8 Hz, 1H), 3.29 (s, 3H), 2.78-2.58 (m, 3H), 2.29 (t, J=1.9 Hz, 1H), 2.00-1.78 (m, 2H), 1.67-1.48 (m, 4H), 1.48-1.38 (m, 1H), 1.34 (d, J=6.8 Hz, 6H), 0.89 (dq, J=6.3, 3.7 Hz, 2H), 0.83-0.70 (m, 6H). ESI MS [M+H]⁺ for C₃₃H₃₉FN₇O₂, calcd 584.3, found 584.3.

### Example 58: N-[6-cyclopropyl-4-[4-fluoro-2-(4-methyl-1,2,4-triazol-3-yl)phenyl]pyridin-2-yl]-5-[[(3S)-3-methylpiperidin-1-yl]methyl]-2-oxo-1-(2,2,2-trifluoroethyl)pyridine-3-carboxamide

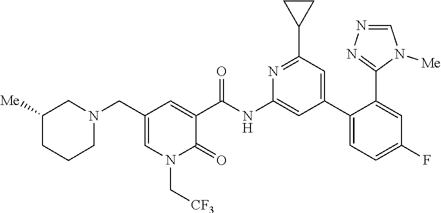

The title compound was prepared in a similar fashion to that described for Example 56 using (S)-3-methylpiperidine for the reductive amination step and 1,1,1-trifluoro-2-iodoethane for N-alkylation. ¹H NMR (400 MHz, DMSO-d₆) δ 12.01 (s, 1H), 8.49-8.36 (m, 2H), 7.99 (s, 1H), 7.75-7.61 (m, 2H), 7.61-7.48 (m, 2H), 6.69 (d, J=1.4 Hz, 1H), 5.06 (q, J=9.0 Hz, 2H), 3.28 (s, 3H), 2.76-2.59 (m, 3H), 2.32-2.26 (m, 1H), 1.93 (td, J=8.3, 4.3 Hz, 1H), 1.83 (t, J=11.1 Hz, 1H), 1.66-1.49 (m, 4H), 1.48-1.32 (m, 1H), 0.89 (dt, J=8.1, 3.1 Hz, 2H), 0.84-0.72 (m, 6H). ESI MS [M+H]⁺ for C₃₂H₃₄F₄N₇O₂, calcd 624.3, found 624.2.

### Example 59: N-[6-cyclopropyl-4-[4-fluoro-2-(4-methyl-1,2,4-triazol-3-yl)phenyl]pyridin-2-yl]-1-(2-hydroxyethyl)-5-[[(3S)-3-methylpiperidin-1-yl]methyl]-2-oxopyridine-3-carboxamide

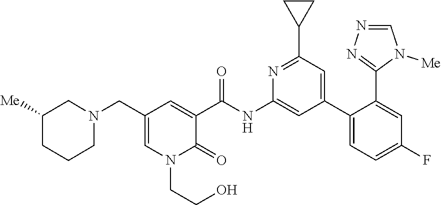 [SMILES: C[C@H]1CCCN(Cc2cc(C(=O)Nc3cc(-c4ccc(F)cc4-c4nncn4C)cc(C4CC4)n3)c(=O)n(CCO)c2)C1]

The title compound was prepared in a similar fashion to that described for Example 56 using (S)-3-methylpiperidine for the reductive amination step and 2-bromoethanol for N-alkylation. ¹H NMR (400 MHz, DMSO-d₆) δ 12.35 (s, 1H), 8.46 (s, 1H), 8.36 (s, 1H), 7.89 (s, 1H), 7.74-7.64 (m, 2H), 7.60-7.48 (m, 2H), 6.67 (s, 1H), 4.96-4.83 (m, 1H), 4.10 (d, J=5.0 Hz, 2H), 3.67 (q, J=5.3 Hz, 2H), 3.25 (s, 3H), 2.80-2.62 (m, 3H), 2.32-2.25 (m, 1H), 1.99-1.89 (m, 2H), 1.67-1.37 (m, 5H), 0.93-0.85 (m, 2H), 0.85-0.69 (m, 6H). ESI MS [M+H]⁺ for C₃₂H₃₇FN₇O₃, calcd 586.3, found 586.3.

### Example 60: N-[6-cyclopropyl-4-[4-fluoro-2-(4-methyl-1,2,4-triazol-3-yl)phenyl]pyridin-2-yl]-5-[[(3S)-3-methylpiperidin-1-yl]methyl]-1-(oxan-4-ylmethyl)-2-oxopyridine-3-carboxamide

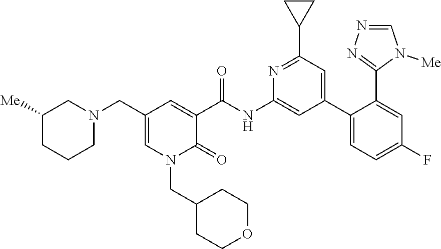 [SMILES: C[C@H]1CCCN(Cc2cc(C(=O)Nc3cc(-c4ccc(F)cc4-c4nncn4C)cc(C4CC4)n3)c(=O)n(CC3CCOCC3)c2)C1]

The title compound was prepared in a similar fashion to that described for Example 56 using (S)-3-methylpiperidine for the reductive amination step and 4-bromomethyltetrahydropyran for N-alkylation. ¹H NMR (400 MHz, DMSO-d₆) δ 12.35 (s, 1H), 8.46 (s, 1H), 8.35 (d, J=2.5 Hz, 1H), 7.98 (d, J=2.5 Hz, 1H), 7.78-7.62 (m, 2H), 7.62-7.44 (m, 2H), 6.67 (d, J=1.4 Hz, 1H), 3.95 (d, J=7.3 Hz, 2H), 3.80 (d, J=11.0 Hz, 2H), 3.28-3.15 (m, 6H), 2.76-2.61 (m, 2H), 2.32-2.26 (m, 1H), 2.13-2.00 (m, 1H), 1.94 (ddd, J=13.0, 8.5, 4.8 Hz, 1H), 1.88-1.74 (m, 1H), 1.65-1.47 (m, 4H), 1.46-1.33 (m, 3H), 1.33-1.19 (m, 2H), 0.93-0.85 (m, 2H), 0.84-0.71 (m, 6H). ESI MS [M+H]⁺ for C₃₆H₄₃FN₇O₃, calcd 640.3, found 640.3.

### Example 61: N-[6-cyclopropyl-4-[4-fluoro-2-(4-methyl-1,2,4-triazol-3-yl)phenyl]pyridin-2-yl]-5-[[(3S)-3-methylpiperidin-1-yl]methyl]-1-(2-methylpropyl)-2-oxopyridine-3-carboxamide

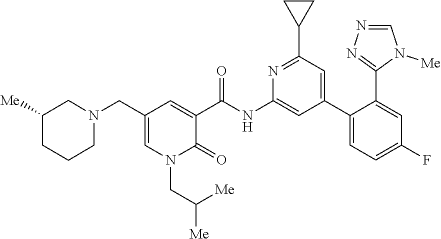

The title compound was prepared in a similar fashion to that described for Example 56 using (S)-3-methylpiperidine for the reductive amination step and 1-bromo-2-methylpropane for N-alkylation. ¹H NMR (400 MHz, DMSO-d₆) δ 12.36 (s, 1H), 8.46 (s, 1H), 8.35 (d, J=2.6 Hz, 1H), 7.97 (d, J=2.6 Hz, 1H), 7.72 (d, J=1.4 Hz, 1H), 7.68 (dd, J=8.6, 5.6 Hz, 1H), 7.55 (ddd, J=18.6, 9.0, 2.8 Hz, 2H), 6.66 (d, J=1.4 Hz, 1H), 3.87 (d, J=7.4 Hz, 2H), 3.27 (s, 3H), 2.74-2.59 (m, 3H), 2.31-2.26 (m, 1H), 2.11 (dt, J=13.4, 6.8 Hz, 1H), 1.93 (tt, J=8.3, 4.7 Hz, 1H), 1.81 (t, J=11.3 Hz, 1H), 1.66-1.47 (m, 4H), 1.47-1.31 (m, 1H), 0.89 (dd, J=8.0, 2.9 Hz, 2H), 0.85 (d, J=6.7 Hz, 6H), 0.77 (t, J=6.1 Hz, 6H). ESI MS [M+H]⁺ for C₃₄H₄₁FN₇O₂, calcd 598.3, found 598.3.

### Example 62: 1-(cyclobutylmethyl)-N-[6-cyclopropyl-4-[4-fluoro-2-(4-methyl-1,2,4-triazol-3-yl)phenyl]pyridin-2-yl]-5-[[(3S)-3-methylpiperidin-1-yl]methyl]-2-oxopyridine-3-carboxamide

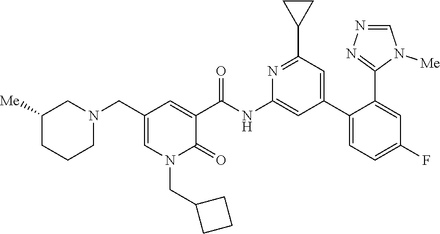 [SMILES: C[C@H]1CCCN(Cc2cc(C(=O)Nc3cc(-c4ccc(F)cc4-c4nncn4C)cc(C4CC4)n3)c(=O)n(CC3CCC3)c2)C1]

The title compound was prepared in a similar fashion to that described for Example 56 using (S)-3-methylpiperidine for the reductive amination step and (bromomethyl)cyclobutane for N-alkylation. ¹HNMR (400 MHz, DMSO-d₆) δ 12.35 (s, 1H), 8.45 (s, 1H), 8.33 (s, 1H), 8.02 (s, 1H), 7.74-7.64 (m, 2H), 7.55 (ddd, J=18.7, 8.9, 2.8 Hz, 2H), 6.67 (s, 1H), 4.08 (d, J=7.4 Hz, 2H), 3.27 (s, 3H), 3.07-2.80 (m, 2H), 2.81-2.59 (m, 3H), 2.31-2.24 (m, 1H), 1.99-1.86 (m, 3H), 1.86-1.69 (m, 5H), 1.68-1.32 (m, 4H), 0.89 (d, J=7.7 Hz, 2H), 0.84-0.66 (m, 6H), ESI MS [M+H]⁺ for C₃₅H₄₁FN₇O₂, calcd 610.3, found 610.3.

### Example 63: N-{4-[4-cyano-2-(4-cyano-1-methyl-2-imidazolyl)phenyl]-6-cyclopropyl-2-pyridyl}-1-(cyclopropylmethyl)-5-[(2-methoxyethylamino)methyl]-2-oxo-1,2-dihydronicotinamide

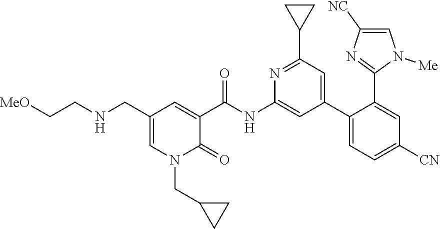 [SMILES: COCCNCc1cc(C(=O)Nc2cc(-c3ccc(C#N)cc3-c3nc(C#N)cn3C)cc(C3CC3)n2)c(=O)n(CC2CC2)c1]

The title compound was prepared in a similar fashion to that described for Example 49 and Example 56. ¹H NMR (400 MHz, CDCl₃) δ 12.40 (s, 1H), 8.55 (d, J=2.6 Hz, 1H), 8.13 (d, J=1.5 Hz, 1H), 7.90 (dq, J=3.8, 1.8 Hz, 2H), 7.76 (d, J=8.5 Hz, 1H), 7.73 (d, J=2.7 Hz, 1H), 7.37 (s, 1H), 6.45 (d, J=1.5 Hz, 1H), 3.94 (d, J=7.2 Hz, 2H), 3.74-3.65 (m, 2H), 3.56-3.47 (m, 2H), 3.37 (s, 3H), 3.13 (s, 3H), 2.85-2.74 (m, 2H), 1.92-1.83 (m, 1H), 1.43-1.27 (m, 1H), 1.00-0.90 (m, 4H), 0.73-0.61 (m, 2H), 0.45 (dt, J=6.2, 4.8 Hz, 2H). ESI MS [M+H]⁺ for C₃₄H₃₅N₈O₃, calcd 603.3, found 603.1.

### Example 64: N-{4-[4-cyano-2-(5-methyl-1,3-oxazol-4-yl)phenyl]-6-cyclopropyl-2-pyridyl}-1-(cyclopropylmethyl)-5-[(2-methoxyethylamino)methyl]-2-oxo-1,2-dihydronicotinamide

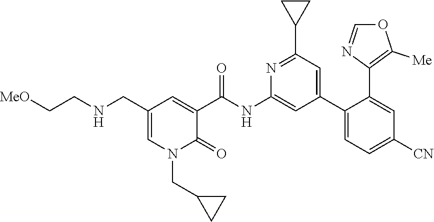 [SMILES: COCCNCc1cc(C(=O)Nc2cc(-c3ccc(C#N)cc3-c3ncoc3C)cc(C3CC3)n2)c(=O)n(CC2CC2)c1]

The title compound was prepared in a similar fashion to that described for Examples 51, 52 and 56. ¹H NMR (400 MHz, CDCl₃) δ 12.31 (s, 1H), 8.54 (d, J=2.7 Hz, 1H), 8.10 (d, J=1.4 Hz, 1H), 7.86 (d, J=1.7 Hz, 1H), 7.79 (s, 1H), 7.73 (dd, J=8.1, 1.9 Hz, 2H), 7.61 (d, J=8.1 Hz, 1H), 6.62 (d, J=1.5 Hz, 1H), 3.93 (m, 2H), 3.71 (s, 2H), 3.58-3.46 (m, 2H), 3.37 (s, 3H), 2.82 (t, J=5.0 Hz, 2H), 1.87 (m, 4H), 1.49-1.29 (m, 1H), 1.03-0.81 (m, 4H), 0.66 (ddd, J=8.2, 5.9, 4.5 Hz, 2H), 0.53-0.32 (m, 2H). ESI MS [M+H]⁺ for C₃₃H₃₅N₆O₄, calcd 579.3, found 579.1.

### Example 65: N-(6-cyclopropyl-4-{4-fluoro-2-[(3-fluoro-1-azetidinyl)carbonyl]phenyl}-2-pyridyl)-5-{[(S)-3-methyl-1-piperidyl]methyl}-1-cyclopropyl-2-oxo-1,2-dihydronicotinamide

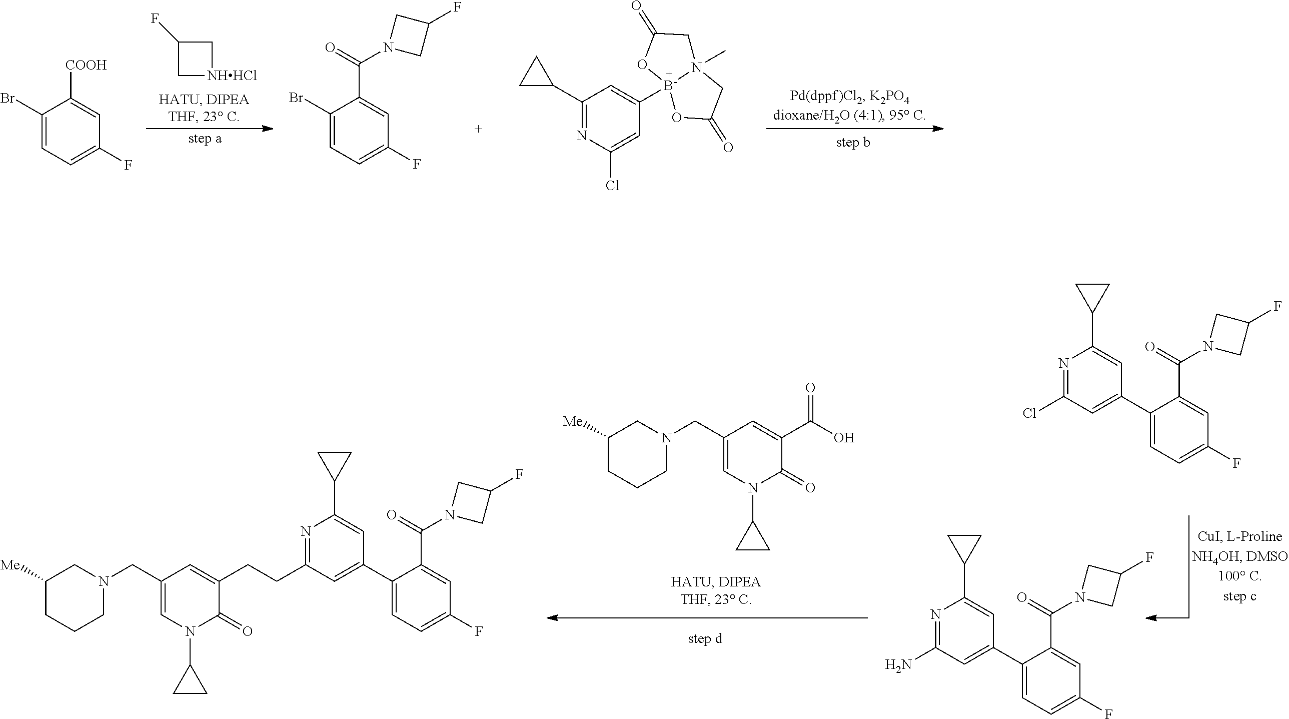

Step a: To a solution of 2-bromo-5-fluorobenzoic acid (3.0 g, 13.7 mmol, 1 equiv.) in THF (68 mL, 0.2 M) were added DIPEA (4.8 mL, 27.4 mmol, 2 equiv.), HATU (6.7 g, 17.8 mmol, 1.3 equiv.) and 3-fluoroazetidine hydrochloride (2.3 g, 20.54 mmol, 1.5 equiv.). The reaction mixture was stirred overnight at room temperature and diluted with EtOAc (100 mL) and water (150 mL). The organic phase was separated, washed with brine (100 mL), dried over Na₂SO₄ and concentrated to dryness under reduced pressure. The crude residue was purified by silica gel flash column chromatography (0-10% MeOH gradient in CH₂Cl₂) to afford the desired product.

Step b: A 40 mL vial was charged with 1-(2-chloro-6-cyclopropylpyridin-4-yl)-5-methyl-2,8-dioxa-5-azonia-1-boranuidabicyclo[3.3.0]octane-3,7-dione (0.62 mg, 2.0 mmol, 1 equiv.), (2-bromo-5-fluorophenyl)-(3-fluoroazetidin-1-yl)methanone (0.55 g, 2.0 mmol. 1 equiv.) and K₃PO₄ (1.27 g, 6.0 mmol, 3 equiv.). The reagents were suspended in a mixture of dioxane/water (20 mL, 4:1 v/v, 0.1 M) and the resulting solution was sparged with N₂ for 10 minutes. Then Pd(dppf)Cl₂ (146 mg, 0.2 mmol, 10%) was added, and the mixture was heated to 95° C. for 6 hours. The reaction mixture was partitioned between EtOAc (50 mL) and water (50 mL). The organic phase was separated, and the aqueous phase was additionally extracted with EtOAc (3×20 mL). The combined organics were dried over Na₂SO₄ and concentrated to dryness under reduced pressure. The crude residue was purified via column chromatography (SiO₂, 0-50% EtOAc gradient in hexane) to afford[2-(2-chloro-6-cyclopropylpyridin-4-yl)-5-fluorophenyl]-(3-fluoroazetidin-1-yl)methanone as the desired product.

Step c: To a solution of the product from step b (0.5 g, 1.47 mmol, 1.0 equiv.) and aq. conc. NH₄OH (1.85 g, 14.7 mmol, 10 equiv.) in DMSO (7.4 mL, 0.2M) were added CuI (0.28 mg, 1.47 mmol, 1.0 equiv.), and L-proline (0.34 g, 2.94 mmol, 2.0 equiv.). The resulting mixture was heated at 100° C. for 16 h. After cooling to room temperature, the reaction mixture was diluted with EtOAc (50 mL) and washed with brine (50 mL). The organic phase was separated, dried over Na₂SO₄ and concentrated to dryness under vacuum. The crude residue was purified by column chromatography (SiO₂, 0-40% EtOAc gradient in hexanes) to produce t [2-(2-amino-6-cyclopropylpyridin-4-yl)-5-fluorophenyl]-(3-fluoroazetidin-1-yl)methanone as the desired product.

Step d: To a solution of a product from step c, (40 mg, 0.12 mmol, 1 equiv.) and 1-cyclopropyl-5-[[(3S)-3-methylpiperidin-1-yl]methyl]-2-oxopyridine-3-carboxylic acid (35.0 mg, 0.24 mmol, 1.0 equiv., obtained according to the protocol described for Example 1). in DMF (1 mL, 0.1 M) were added DIPEA (42 mL, 0.24 mmol, 2 equiv.), and HATU (91 mg, 0.24 mmol, 2.0 equiv.). The reaction mixture was stirred overnight at room temperature. After completion, the mixture was directly fractionated by preparative HPLC (20-90% CH₃CN gradient in water, 0.1% formic acid) to afford the title compound. ¹H NMR (400 MHz, CDCl₃) δ 12.20 (s, 1H), 8.50 (d, J=2.6 Hz, 1H), 8.18 (d, J=1.4 Hz, 1H), 7.85 (d, J=2.7 Hz, 1H), 7.51 (dd, J=9.3, 5.3 Hz, 1H), 7.24-7.16 (m, 2H), 6.99 (d, J=1.4 Hz, 1H), 5.33-5.15 (m, 1H), 4.38-4.25 (m, 1H), 4.18-3.92 (m, 2H), 3.90-3.78 (m, 1H), 3.61 (s, 2H), 3.47 (tt, J=7.6, 4.2 Hz, 1H), 3.12-2.91 (m, 2H), 2.24-2.15 (m, 1H), 2.06-1.96 (m, 1H), 1.93-1.85 (m, 1H), 1.81-1.56 (m, 4H), 1.11-1.06 (m, 2H), 1.03-0.92 (m, 4H), 0.91-0.80 (m, 6H). ESI MS [M+H]⁺ for C₃₄H₃₇F₂N₅O₃, calcd 602.2, found 602.2.

### Example 66: N-{5-cyano-4′-fluoro-2′-[(3-fluoro-1-azetidinyl)carbonyl]-3-biphenylyl}-5-{[(S)-3-methyl-1-piperidyl]methyl}-1-cyclopropyl-2-oxo-1,2-dihydronicotinamide

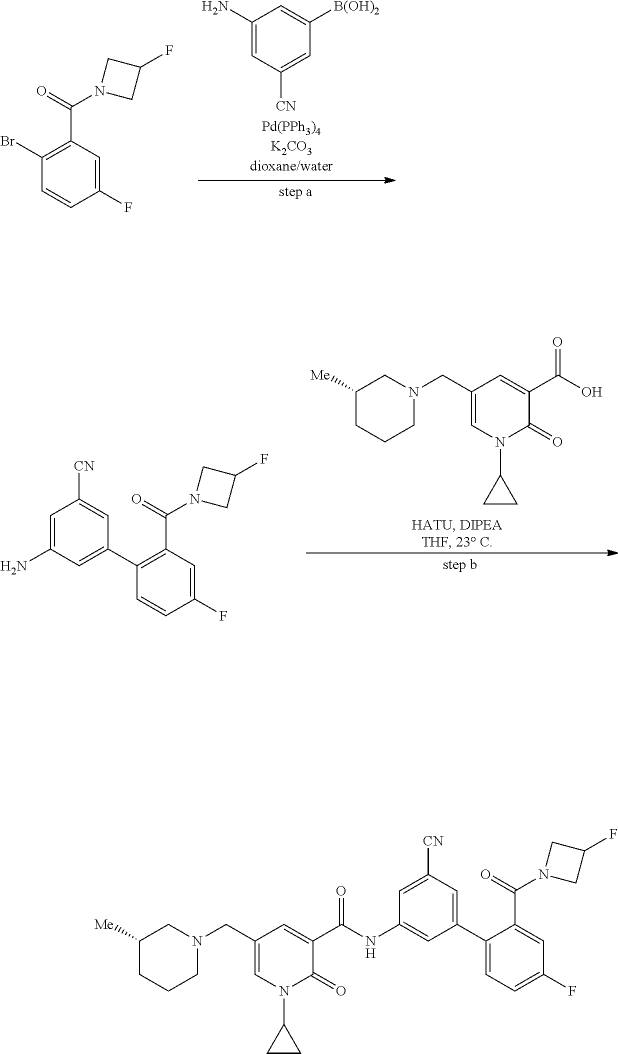 [SMILES: O=C(c1cc(F)ccc1Br)N1CC(F)C1 ; N#Cc1cc(N)cc(B(O)O)c1 ; N#Cc1cc(N)cc(-c2ccc(F)cc2C(=O)N2CC(F)C2)c1 ; C[C@H]1CCCN(Cc2cc(C(=O)O)c(=O)n(C3CC3)c2)C1 ; C[C@H]1CCCN(Cc2cc(C(=O)Nc3cc(C#N)cc(-c4ccc(F)cc4C(=O)N4CC(F)C4)c3)c(=O)n(C3CC3)c2)C1]

Step a: A 40 mL vial was charged with (2-bromo-5-fluorophenyl)-(3-fluoroazetidin-1-yl)methanone (0.5 g, 1.82 mmol, 1 equiv.), (3-amino-5-cyanophenyl)boronic acid (0.3 g, 1.82 mmol. 1.0 equiv.) and K₂CO₃ (0.75 g, 5.4 mmol, 3 equiv.). The reagents were suspended in a dioxane/water mixture (10 mL, 3:1 v/v), and the resulting solution was sparged with N₂ for 10 minutes. Then Pd(PPh₃)₄ (210 mg, 0.182 mmol, 0.1 equiv.) was added, and the mixture was heated to 95° C. for 4 hours. The reaction mixture was partitioned between EtOAc (30 mL) and water (30 mL), the organic phase was separated, and the aqueous layer was additionally extracted with EtOAc (2×15 mL). The combined organic phase was dried over Na₂SO₄ and concentrated to dryness under vacuum. The crude residue was purified via column chromatography (SiO₂, 0-100% EtOAc gradient in hexanes) to afford the desired coupling product.

Step b: The reaction was performed in a similar fashion to Example 66), step d. ¹H NMR (400 MHz, CDCl₃) δ 12.40 (s, 1H), 8.50 (d, J=2.7 Hz, 1H), 8.14 (t, J=1.9 Hz, 1H), 8.05 (dd, J=2.1, 1.4 Hz, 1H), 8.00 (d, J=2.6 Hz, 1H), 7.48-7.38 (m, 2H), 7.25-7.19 (m, 2H), 5.36-5.15 (m, 1H), 4.40-4.25 (m, 1H), 4.19-4.05 (m, 1H), 4.03-3.90 (m, 1H), 3.90-3.77 (m, 1H), 3.72-3.62 (m, 2H), 3.50-3.42 (m, 1H), 3.19-3.00 (m, 2H), 2.27-2.17 (m, 1H), 2.00-1.69 (m, 4H), 1.32-1.21 (m, 4H), 1.04-0.96 (m, 2H), 0.91 (d, J=6.1 Hz, 3H). ESI MS [M+H]⁺ for C₃₃H₃₃F₂N₅O₃, calcd 586.2, found 586.2.

### Example 67: N-(6-cyclopropyl-4-{4-fluoro-2-[(3-fluoro-1-azetidinyl)carbonyl]phenyl}-2-pyridyl)-1-cyclopropyl-5-[(2-methoxyethylamino)methyl]-2-oxo-1,2-dihydronicotinamide

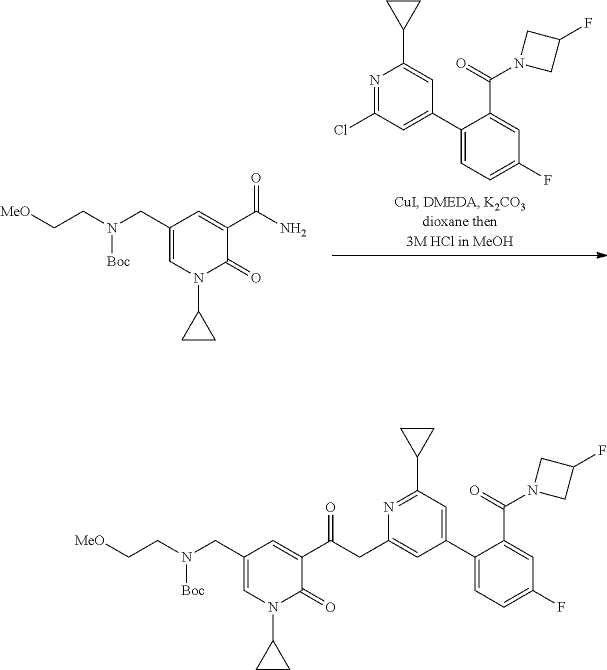 [SMILES: COCCN(Cc1cc(C(N)=O)c(=O)n(C2CC2)c1)C(=O)OC(C)(C)C ; O=C(c1cc(F)ccc1-c1cc(Cl)nc(C2CC2)c1)N1CC(F)C1 ; COCCN(Cc1cc(C(=O)Cc2cc(-c3ccc(F)cc3C(=O)N3CC(F)C3)cc(C3CC3)n2)c(=O)n(C2CC2)c1)C(=O)OC(C)(C)C]

To a solution of the tert-butyl N-[(5-carbamoyl-1-cyclopropyl-6-oxopyridin-3-yl)methyl]-N-(2-methoxyethyl)carbamate (48 mg, 0.13 mmol, 1.0 equiv., prepared as described in Example 3) and [2-(2-chloro-6-cyclopropylpyridin-4-yl)-5-fluorophenyl]-(3-fluoroazetidin-1-yl)methanone (45 mg, 0.13 mmol, 1.0 equiv., prepared according to Example 66) in dioxane (2.6 mL, 0.05M) was added CuI (25 mg, 0.13 mmol, 1.0 equiv.), N,N′-dimethylethylenediamine (23.0 mg, 0.26 mmol, 2.0 equiv.), and K₂CO₃ (54 mg, 0.34 mmol, 3.0 equiv.). The resulting mixture was heated at 110° C. for 12 h in a sealed vial. The resulting mixture was cooled to room temperature and diluted with EtOAc (20 mL) and aq. NH₄Cl (10 mL). The organic phase was separated, and the aqueous phase was additionally extracted with EtOAc (2×10 mL). The combined organic extract was dried over Na₂SO₄ and concentrated under reduced pressure to yield the corresponding crude coupling product. The crude product was dissolved in HCl in MeOH (3 mL, 3 M), and the resulting solution was stirred at room temperature for 3 h, then concentrated to dryness under reduced pressure. The crude product was directly purified by reversed phase preparative HPLC (C18, 20-90% CH₃CN gradient in water with 0.1% formic acid) to afford the title compound. ¹H NMR (400 MHz, CDCl₃) δ 12.27 (s, 1H), 8.53 (d, J=2.6 Hz, 1H), 8.19 (d, J=1.5 Hz, 1H), 7.65 (d, J=2.6 Hz, 1H), 7.56-7.44 (m, 1H), 7.24-7.12 (m, 2H), 6.99 (d, J=1.5 Hz, 1H), 5.36-5.17 (m, 1H), 4.39-4.26 (m, 1H), 4.17-3.96 (m, 2H), 3.93-3.78 (m, 1H), 3.69-3.65 (m, 3H), 3.56-3.41 (m, 2H), 3.37 (s, 3H), 2.81 (dd, J=5.5, 4.6 Hz, 2H), 2.07-1.94 (m, 1H), 1.74 (s, 1H), 1.26-1.23 (m, 2H), 1.11-1.07 (m, 2H), 1.00-0.91 (m, 4H).

### Example 68: N-(6-cyclopropyl-4-{2-[(3,3-difluoro-1-azetidinyl)carbonyl]-4-fluorophenyl}-2-pyridyl)-1-cyclopropyl-5-[(2-methoxyethylamino)methyl]-2-oxo-1,2-dihydronicotinamide

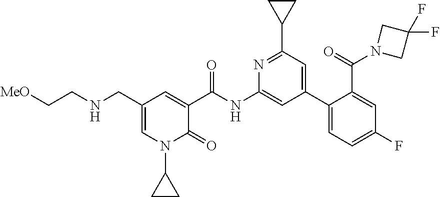 [SMILES: COCCNCc1cc(C(=O)Nc2cc(-c3ccc(F)cc3C(=O)N3CC(F)(F)C3)cc(C3CC3)n2)c(=O)n(C2CC2)c1]

The title compound was prepared in a similar fashion to that described for Example 66 and Example 68 starting from 3,3-difluoroazetidine hydrochloride. ¹H NMR (400 MHz, CDCl₃) δ 12.27 (s, 1H), 8.55 (d, J=2.7 Hz, 1H), 8.24 (d, J=1.5 Hz, 1H), 7.65 (d, J=2.6 Hz, 1H), 7.59-7.48 (m, 1H), 7.26-7.14 (m, 2H), 6.97 (d, J=1.5 Hz, 1H), 4.33 (t, J=11.9 Hz, 2H), 3.95 (t, J=11.6 Hz, 2H), 3.67 (s, 2H), 3.58-3.41 (m, 3H), 3.37 (s, 3H), 2.80 (dd, J=5.4, 4.6 Hz, 2H), 2.06-1.96 (m, 1H), 1.79 (s, 1H), 1.30-1.20 (m, 2H), 1.13-1.06 (m, 2H), 1.01-0.89 (m, 4H). ESI MS [M+H]+ for C₃₁H₃₂F₃N₅O₄ calcd 596.2, found 596.2.

### Example 69: N-{5-cyclopropyl-2′-[(3,3-difluoro-1-azetidinyl)carbonyl]-4′-fluoro-3-biphenylyl}-1-cyclopropyl-5-[(2-methoxyethylamino)methyl]-2-oxo-1,2-dihydronicotinamide

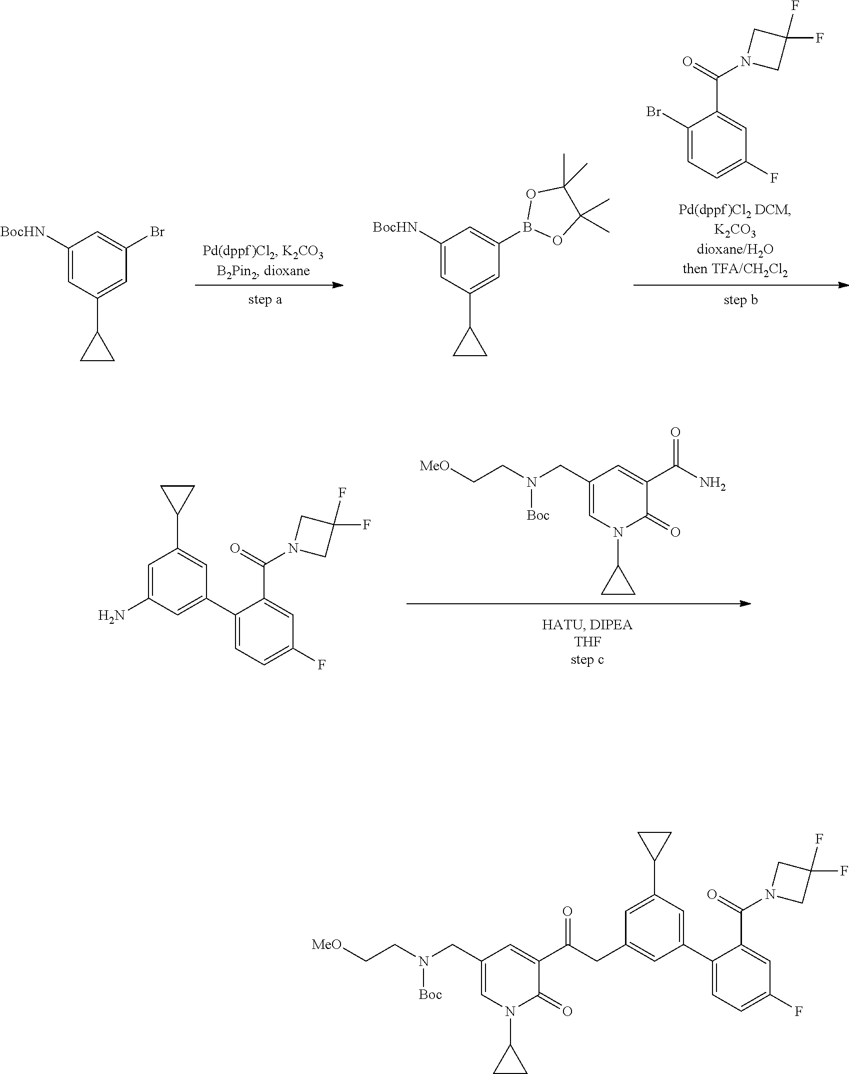 [SMILES: CC(C)(C)OC(=O)Nc1cc(Br)cc(C2CC2)c1 ; CC(C)(C)OC(=O)Nc1cc(B2OC(C)(C)C(C)(C)O2)cc(C2CC2)c1 ; O=C(c1cc(F)ccc1Br)N1CC(F)(F)C1 ; Nc1cc(-c2ccc(F)cc2C(=O)N2CC(F)(F)C2)cc(C2CC2)c1 ; COCCN(Cc1cc(C(N)=O)c(=O)n(C2CC2)c1)C(=O)OC(C)(C)C ; COCCN(Cc1cc(C(=O)Cc2cc(-c3ccc(F)cc3C(=O)N3CC(F)(F)C3)cc(C3CC3)c2)c(=O)n(C2CC2)c1)C(=O)OC(C)(C)C]

Step a: A 40 mL vial was charged with the tert-butyl N-(3-bromo-5-cyclopropylphenyl)carbamate (0.5 g, 2.3 mmol, 1.0 equiv.), bis(pinacolato)diboron (0.42 mg, 3.5 mmol, 1.2 equiv.), KOAc (0.7 g, 7.0 mmol, 3.0 equiv.) and Pd(dppf)Cl₂·CH₂Cl₂ (0.19 g, 0.23 mmol, 0.1 equiv.). Dry dioxane (15 mL, 0.2 M) was added, the reaction mixture was sparged with N₂ for 10 minutes and heated to 90° C. overnight. The resulting solution was cooled to ambient temperature and diluted with brine (10 mL) and EtOAc (75 mL). The organic phase was separated and the aqueous phase was additionally extracted with EtOAc (75 mL). The combined organic extract was dried over Na₂SO₄, filtered, and concentrated to dryness under reduced pressure. The crude residue was purified by silica gel flash column chromatography (0 to 50% EtOAc/hexane) to afford the BPin derivative.

Step b: A 40 mL vial was charged with the tert-butyl N-[3-cyclopropyl-5-(4,4,5,5-tetramethyl-1,3,2-dioxaborolan-2-yl)phenyl]carbamate (0.24 g, 0.93 mmol, 1.1 equiv.), (2-bromo-5-fluorophenyl)-(3,3-difluoroazetidin-1-yl)methanone (0.25 g, 0.85 mmol, 1.0 equiv.) and K₂CO₃ (0.35 g, 2.55 mmol, 3 equiv.). The reagents were suspended in a mixture of dioxane/water (8 mL, 4:1 v/v) and the resulting solution was sparged with N₂ for 10 minutes. Then Pd(dppf)Cl₂·CH₂Cl₂ (69 mg, 0.085 mmol, 0.1 equiv.) was added, and the mixture was heated to 95° C. for 8 h. The reaction mixture was cooled to room temperature and partitioned between EtOAc (50 mL) and water (10 mL). The organic phase was separated, and the aqueous phase was additionally extracted with EtOAc (2×25 mL). The combined organic extract was dried over Na₂SO₄ and concentrated under reduced pressure to yield the corresponding crude coupling product. The resulting crude residue was dissolved in CH₂Cl₂ (3 mL), and trifluoroacetic acid (1 mL) was added. The resulting solution was stirred at 23° C. for 3 h. The solvent was removed under vacuum, and the resulting residue was purified via column chromatography (SiO₂, 0-10% MeOH gradient in CH₂Cl₂) to afford [2-(3-amino-5-cyclopropylphenyl)-5-fluorophenyl]-(3,3-difluoroazetidin-1-yl)methanone as the desired product.

Step c: The title compound was prepared in a similar fashion to that described for Example 66, step d. ¹H NMR (400 MHz, CDCl₃) δ 12.14 (s, 1H), 8.58 (d, J=2.6 Hz, 1H), 7.89 (t, J=1.8 Hz, 1H), 7.66 (d, J=2.6 Hz, 1H), 7.48 (dd, J=8.6, 5.3 Hz, 1H), 7.32 (t, J=1.8 Hz, 1H), 7.25-7.15 (m, 2H), 6.89 (t, J=1.7 Hz, 1H), 4.27 (t, J=11.9 Hz, 2H), 3.79 (t, J=11.7 Hz, 2H), 3.69 (s, 2H), 3.51 (dd, J=5.5, 4.5 Hz, 2H), 3.46-3.39 (m, 1H), 3.37 (s, 3H), 2.84-2.76 (m, 2H), 1.96-1.88 (m, 1H), 1.65 (s, 1H), 1.27-1.23 (m, 2H), 1.00-0.96 (m, 4H), 0.75-0.69 (m, 2H). ESI MS [M+H]+ for C₃₂H₃₃F₃N₄O₄, calcd 596.2, found 596.2.

### Example 70: 1-Cyclopropyl-N-[6-cyclopropyl-4-[4-fluoro-2-(5-methylpyrazol-1-yl)phenyl]pyridin-2-yl]-5-[(2-methoxyethylamino)methyl]-2-oxopyridine-3-carboxamide

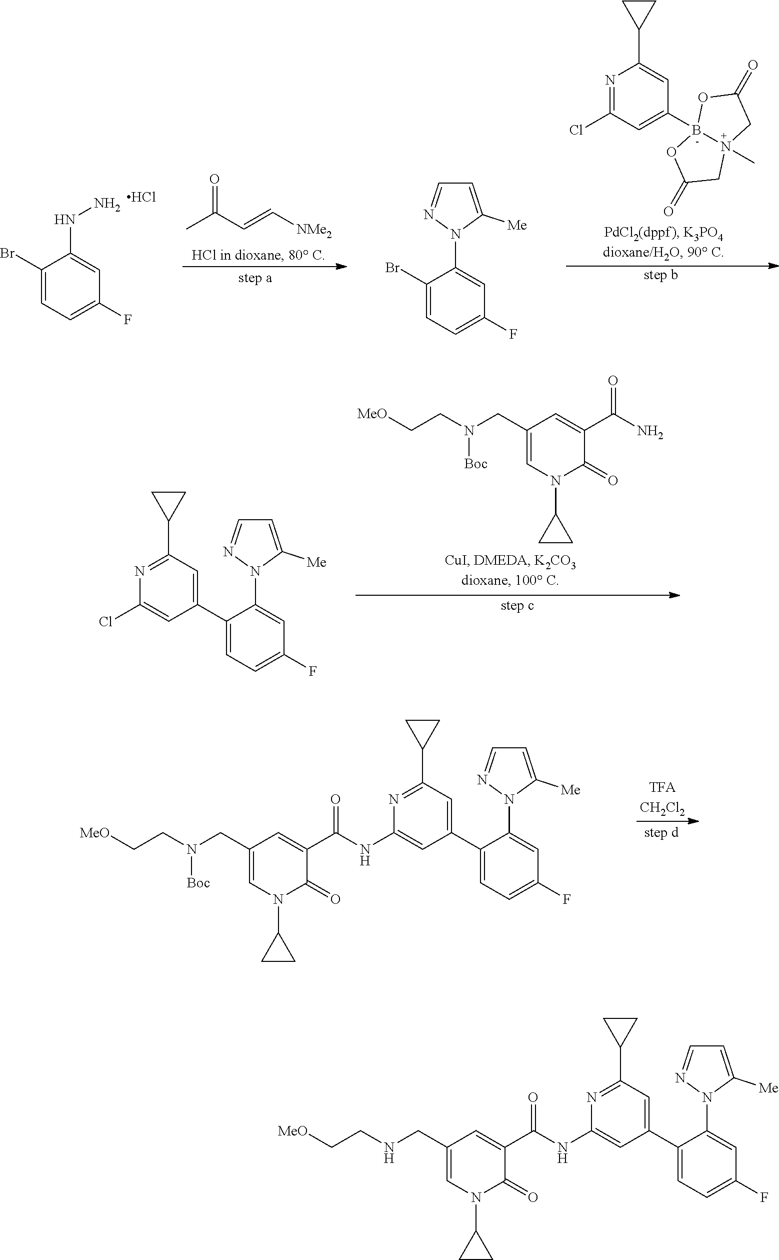

Step a: To the solution of (2-bromo-5-fluorophenyl)hydrazine hydrochloride (0.20 g, 0.97 mmol, 1.0 equiv.) 4-(dimethylamino)-3-buten-2-one (0.12 g, 1.02 mmol, 1.05 equiv.) was added followed by 4 N HCl in dioxane (5 mL). The reaction was then heated to 80° C. for 10 mins, then cooled to room temperature. The resulting solution was neutralized with sat. aq. sodium bicarbonate to pH˜7 and extracted with EtOAc (2×5 mL). The combined organic extract was dried over Na₂SO₄, filtered, and concentrated to dryness under vacuum. The crude residue was purified via column chromatography (SiO₂, 0-20% EtOAc gradient in hexanes) to afford the desired product.

Step b: A solution of the 1-(2-chloro-6-cyclopropylpyridin-4-yl)-5-methyl-2,8-dioxa-5-azonia-1-boranuidabicyclo[3.3.0]octane-3,7-dione (71 mg, 0.36 mol, 1.0 equiv., prepared according to protocol described for Example 15), biaryl product from step a (0.10 g, 0.39 mol, 1.10 equiv.) and K₃PO₄ (0.23 g, 1.08 mol, 3.0 equiv.) in a dioxane/water mixture (3.60 mL, 4:1 v/v) was purged with N₂ for 10 minutes. Pd(dppf)₂Cl₂ (26 mg, 0.036 mmol, 0.1 equiv.) was then added, and the reaction was heated to 90° C. for 1 h. The resulting mixture was cooled to room temperature, quenched with a ˜1:1 mixture of brine and water (2 mL) and extracted with EtOAc (2×5 mL). The combined organics were dried over Na₂SO₄, filtered, and concentrated under vacuum. The crude residue was first purified via column chromatography (C18, 0-30% EtOAc gradient in hexanes). Then the product was repurified using reversed-phase column chromatography (C18, 10-100% CH₃CN gradient in water with 0.1% TFA) to afford the desired product.

Step c: A solution of tert-butyl N-[(5-carbamoyl-1-cyclopropyl-6-oxopyridin-3-yl)methyl]-N-(2-methoxyethyl)carbamate (60 mg, 0.16 mmol, 1.0 equiv., prepared according to protocol described for Example 3), triazole from step b (70 mg, 0.21 mmol, 1.30 equiv.) and K₂CO₃ (50 mg, 0.36 mmol, 3.0 equiv.) in dioxane (3.2 ml) was purged with nitrogen for 10 min. CuI (23 mg, 0.12 mmol, 1.0 equiv.) and DMEDA (24 μL, 0.24 mmol, 2.0 equiv.) were added sequentially, and the reaction was heated for 16 h at 100° C. The reaction mixture was cooled to room temperature, quenched with aq. NH₄Cl (10 mL) and extracted with EtOAc (2×7 mL). The combined organic phase was washed with water (5 mL) and brine (5 mL), dried over Na₂SO₄, and concentrated to dryness under reduced pressure. The crude residue was purified by reversed phase column chromatography (C18, 10-100% CH₃CN gradient in water) to afford the desired product.

Step d: The product from step c (32 mg, 0.049 mmol, 1.0 equiv) was dissolved in CH₂Cl₂ (0.25 mL) and TFA (0.25 mL) was added. The mixture was stirred for 1 h and then concentrated under reduced pressure. The crude residue was purified by reversed phase column chromatography (C18, 10-100% CH₃CN gradient in water) to yield the title compound. ¹H NMR (400 MHz, CDCl₃) δ 12.22 (s, 1H), 8.54 (d, J=2.5 Hz, 1H), 8.15 (t, J=1.2 Hz, 1H), 7.70-7.59 (m, 2H), 7.56 (d, J=1.7 Hz, 1H), 7.33-7.14 (m, 2H), 6.14 (d, J=1.3 Hz, 1H), 6.00 (s, 1H), 3.67 (s, 2H), 3.51 (t, J=5.0 Hz, 2H), 3.43 (tt, J=7.7, 4.3 Hz, 1H), 3.36 (d, J=1.3 Hz, 3H), 2.81 (t, J=5.0 Hz, 2H), 1.78 (s, 4H), 1.34-1.16 (m, 3H), 0.96 (dd, J=6.6, 4.4 Hz, 2H), 0.83 (d, J=6.6 Hz, 4H). ESI MS [M+H]⁺ for C₃₁H₃₃FN₆O₃, calcd 556.3, found 556.3.

### Example 71: N-{6-cyclopropyl-4-[4-fluoro-2-(5-methyl-1H-1,2,3-triazol-1-yl)phenyl]-2-pyridyl}-1-cyclopropyl-5-[(2-methoxyethylamino)methyl]-2-oxo-1,2-dihydronicotinamide

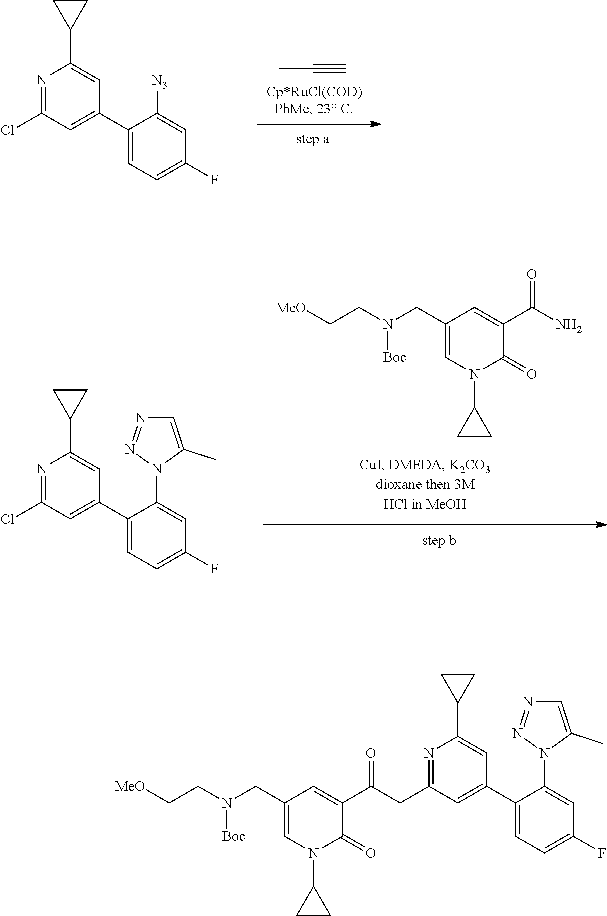

Step a: To a solution of biaryl azide (2.40 g, 8.31 mmol, 1.0 equiv., prepared according to protocol described for Example 73) and propyne (3.33 g, 83.1 mmol, 10 equiv., condensed to the flask at −78° C.) in toluene (70 mL, 0.12 M) in a glass pressure vessel was added Cp*Ru(COD)Cl (0.32 g, 0.83 mmol, 0.1 equiv.) at −78° C. The flask was sealed, and the reaction was stirred for 24 h at room temperature. The resulting solution was concentrated to dryness. The crude product was first purified by column chromatography (SiO₂, 0-60% EtOAc gradient in hexanes), then the second purification was done by reversed-phase column chromatography (C18, 10-100% CH₃CN in water with 0.1% formic acid) to afford corresponding 1,2,3-triazole.

Step b: To a solution of the tert-butyl N-[(5-carbamoyl-1-cyclopropyl-6-oxopyridin-3-yl)methyl]-N-(2-methoxyethyl)carbamate (48 mg, 0.13 mmol, 1.0 equiv., prepared according to protocol described for Example 3) and 2-chloro-6-cyclopropyl-4-[4-fluoro-2-(5-methyltriazol-1-yl)phenyl]pyridine from previous step (43 mg, 0.13 mmol, 1.0 equiv.) in dioxane (2.6 mL, 0.05M) was added CuI (25 mg, 0.13 mmol, 1.0 equiv.), N,N′-dimethylethylenediamine (23.0 mg, 0.26 mmol, 2.0 equiv.) and K₂CO₃ (54 mg, 0.34 mmol, 3.0 equiv.). The resulting mixture was heated overnight at 110° C. in a sealed vial. The mixture was cooled to room temperature and diluted with EtOAc (10 mL) and aq. NH₄Cl (10 mL) and washed with brine. The organic phase was separated, and the aqueous phase was additionally extracted with EtOAc (2×5 mL). The combined organics was dried over Na₂SO₄ and concentrated under reduced pressure to yield the corresponding coupling product. The product was next dissolved in 3 M HCl in MeOH (3 mL) and stirred at room temperature for 3 h. The resulting solution was concentrated to dryness. The crude residue was directly purified by reverse-phase preparative HPLC (C18, 20-90% CH₃CN gradient in water with, 0.1% formic acid) to afford the title compound. ¹H NMR (400 MHz, CDCl₃) δ 12.23 (s, 1H), 8.53 (d, J=2.7 Hz, 1H), 8.10 (d, J=1.4 Hz, 1H), 7.74-7.60 (m, 2H), 7.41 (d, J=0.9 Hz, 1H), 7.40-7.34 (m, 1H), 7.24 (dd, J=8.3, 2.7 Hz, 1H), 6.24 (d, J=1.4 Hz, 1H), 3.69 (s, 2H), 3.56-3.49 (m, 2H), 3.44 (td, J=7.4, 3.8 Hz, 1H), 3.37 (s, 3H), 2.91-2.77 (m, 2H), 2.31 (s, 1H), 1.85 (s, 3H), 1.83-1.71 (m, 1H), 1.31-1.19 (m, 2H), 1.00-0.94 (m, 2H), 0.90-0.81 (m, 4H). ESI MS [M+H]⁺ for C₃₀H₃₂FN₇O₃ calcd 558.2 found 558.2.

### Example 72: 1-Cyclopropyl-N-[6-cyclopropyl-4-[4-fluoro-2-[5-(fluoromethyl)triazol-1-yl]phenyl]pyridin-2-yl]-5-[(2-methoxyethylamino)methyl]-2-oxopyridine-3-carboxamide

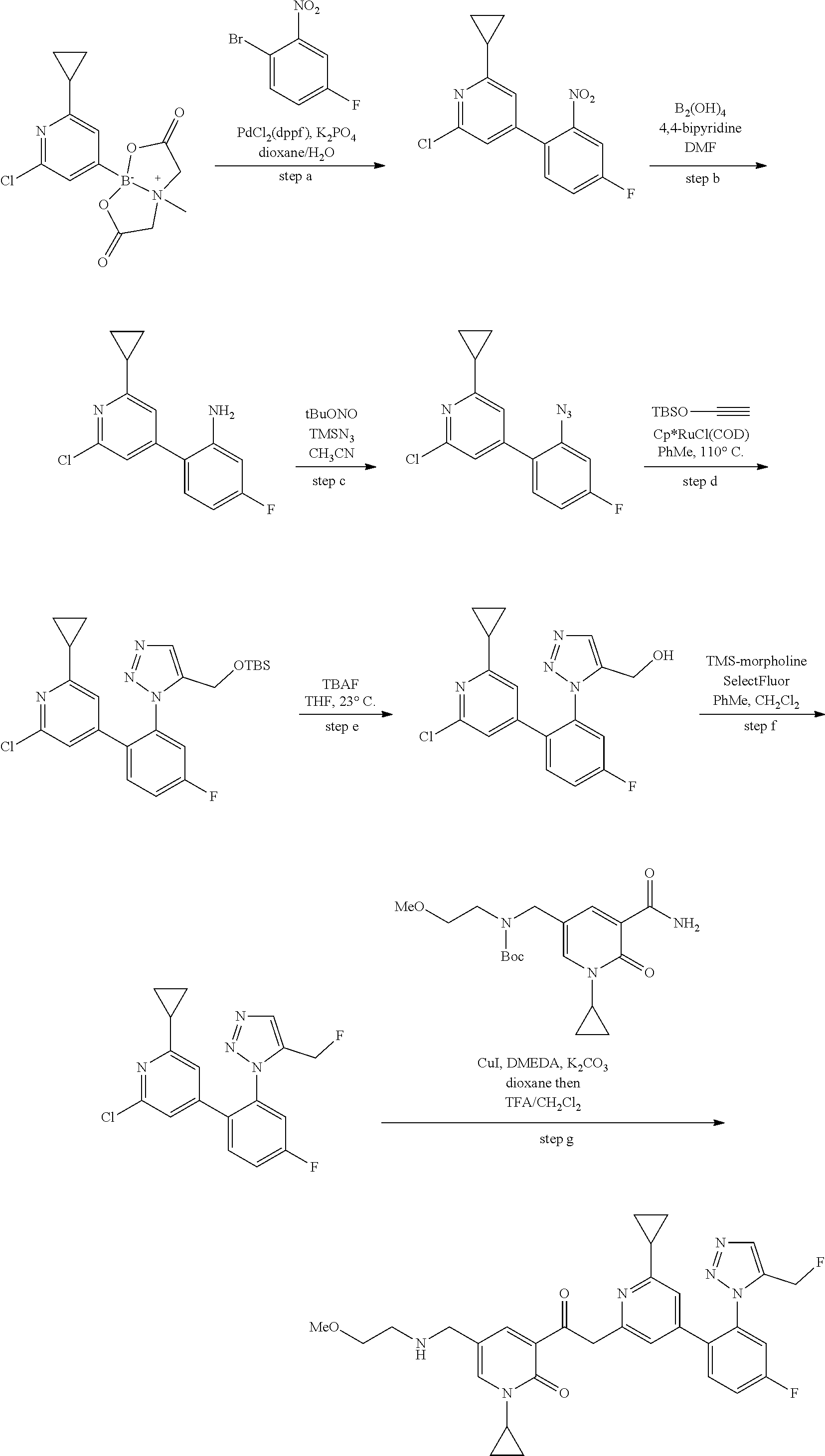

Step a: A suspension pf 1-(2-chloro-6-cyclopropylpyridin-4-yl)-5-methyl-2,8-dioxa-5-azonia-1-boranuidabicyclo[3.3.0]octane-3,7-dione (21.0 g, 0.07 mol, 1.0 equiv., prepared according to protocol described for Example 15), 1-bromo-4-fluoro-2-nitrobenzene (15.7 g, 0.071 mol, 1.05 equiv.) and K₃PO₄ (43.3 g, 0.20 mol, 3.0 equiv.) in dioxane/water mixture (680 mL, 4:1 v/v, 0.1 M) was purged with N₂ for 10 minutes. Pd(dppf)₂Cl₂ (5.0 g, 7.0 mmol, 0.1 equiv.) was added, and the reaction was heated to 90° C. for 1 h under stirring. Then it was cooled to room temperature, diluted with a ˜1:1 mixture of saturated aqueous NaCl/water (300 mL) and extracted with EtOAc (2×400 mL). The combined organics were dried over Na₂SO₄, filtered, and concentrated under vacuum. The crude residue was purified via column chromatography (SiO₂, 0-40% EtOAc gradient in hexanes) to afford the desired biaryl product.

Step b: To a cooled solution of the product from step a (16.4 g, 0.06 mol, 1.0 equiv.) in DMF (280 mL, 0.2 M), tetrahydroxydiboron (15 g, 0.17 mol, 3.0 equiv.) and 4,4-bipyridine (0.88 g, 5.60 mmol, 0.10 equiv.) were added at 0° C. The cooling bath was removed and the mixture was stirred for 1 h at room temperature. Once full conversion was observed by LC/MS analysis the reaction mixture was quenched with water (500 mL), and the product was extracted with EtOAc (2×200 mL). The combined organic extract was dried over Na₂SO₄, filtered, and concentrated under reduced pressure. The crude product was purified by reversed phase column chromatography (C18, 10-100% CH₃CN gradient in water with 0.1% formic acid) to afford the desired aniline product.

Step c: To a solution of the product from step b (14.1 g, 0.054 mol, 1.0 equiv.) in CH₃CN (180 mL, 0.3 M), t-BuONO (8.53 mL, 0.065 mmol, 1.2 equiv.) and TMSN₃ (8.51 mL, 0.065 mol, 1.2 equiv.) were sequentially added at 0° C. The cooling bath was removed, and the resulting mixture solution was stirred at room temperature for 2 h. Once TLC analysis indicated a complete transformation the mixture was concentrated to dryness under reduced pressure. The crude aryl azide was used directly for the next step.

Step d: To a solution of the product from step c (7.16 g, 24.8 mmol, 1.0 equiv.) and t-butyldimethylsilyl propargyl ether (6.34 g, 37.2 mmol, 1.50 equiv.) in toluene (124 mL, 0.2 M) was added Cp*Ru(COD)Cl (0.94 g, 2.48 mmol, 0.1 equiv.). The reaction vial was carefully degassed under vacuum and backfilled with nitrogen 3 times and heated to 110° C. overnight. The resulting solution was cooled to room temperature and concentrated to dryness. The crude product was purified via column chromatography (SiO₂, 0-40% EtOAc gradient in hexanes) to afford corresponding 1,2,3-triazole as a single regioisomer.

Step e: The product from step d (8.0 g, 17.4 mmol, 1.0 equiv.) was dissolved in THF (174 mL, 0.1 M) and TBAF (1.0 M in THF, 23 mL, 1.3 equiv.) was added to the reaction mixture at room temperature. The mixture was stirred for 1 h and quenched with water (50 mL). The product was extracted with EtOAc (2×50 mL). The combined organic extract was dried over Na₂SO₄, concentrated under reduced pressure, and the crude residue was purified by column chromatography (SiO₂, 0-80% EtOAc gradient in hexane) to afford the corresponding benzylic alcohol product.

Step f: TMS-morpholine (7.10 mL, 41.2 mmol, 7.10 equiv.) was added to Deoxo-Fluor® (17.9 g, 40.6 mmol, 7.0 eq, 50% wt solution in PhMe) at 0° C. The ice bath was subsequently removed, and the mixture was warmed to room temperature, during which time it became increasingly heterogeneous, and a white precipitate formed. After 1 h, a solution of the product from step e (2.0 g, 5.80 mmol, 1.0 equiv.) in CH₂Cl₂ (29 mL, 0.2 M) was added to the reaction mixture with pregenerated reagent at 0° C. The reaction was warmed to room temperature, and stirred for 45 min. Upon completion (TLC analysis) the reaction was poured into sat. aq. NaHCO₃ (10 mL) solution, and the product was extracted with CH₂Cl₂ (3×20 mL). The combined organic extract was dried over Na₂SO₄ and concentrated to dryness under reduced pressure. The crude product was purified via column chromatography (SiO₂, 0-80% EtOAc gradient in hexanes) to afford the fluorinated product.

Step g: A solution of tert-butyl N-[(5-carbamoyl-1-cyclopropyl-6-oxopyridin-3-yl)methyl]-N-(2-methoxyethyl)carbamate (43 mg, 0.12 mmol, 1.3 equiv., prepared according to the protocol described for Example 3), fluoromethyltriazole from step f (24 mg, 0.09 mmol, 1.0 equiv.) and K₂CO₃ (37 mg, 0.27 mmol, 3.0 equiv.) in dioxane (2.0 ml) was degassed with a stream of bubbling nitrogen for ten minutes. CuI (17 mg, 0.09 mmol, 1.0 equiv.) and DMEDA (18 μL, 0.18 mmol, 2.0 equiv.) were added, and the reaction was heated for 16 h at 100° C. Then the mixture was cooled to room temperature, diluted with aq. NH₄Cl (10 mL) and extracted with EtOAc (2×10 mL). The combined organic phase was washed with water (2×10 mL) and brine (10 mL), dried over Na₂SO₄, and concentrated to dryness. The crude residue was purified by reversed-phase column chromatography (C18, 10-100% CH₃CN in water with 0.1% formic acid) to afford corresponding coupling product. Then it was dissolved in CH₂Cl₂ (0.30 mL) and TFA (0.3 mL) was added. The mixture was stirred for 1 h and then concentrated to dryness under vacuum. The crude residue was purified by reversed-phase column chromatography (C18, 10-100% CH₃CN in water with 0.1% formic acid) to yield the title compound. ¹H NMR (400 MHz, CDCl₃) δ 12.18 (s, 1H), 8.67-8.42 (m, 1H), 7.93 (s, 1H), 7.82-7.72 (m, 2H), 7.66 (dd, J=8.7, 5.7 Hz, 1H), 7.47-7.35 (m, 1H), 7.34-7.27 (m, 1H), 6.39 (s, 1H), 5.05 (d, J=47.8 Hz, 2H), 3.76 (s, 2H), 3.56 (t, J=5.0 Hz, 2H), 3.43 (dp, J=8.0, 4.3 Hz, 1H), 3.35 (s, 3H), 2.89 (t, J=5.1 Hz, 2H), 1.80 (tt, J=8.7, 5.1 Hz, 1H), 1.38-1.12 (m, 3H), 1.04-0.75 (m, 6H). ESI MS [M+H]⁺ for C₃₀H₃₁F₂N₇O₃ calcd 575.3, found 575.3.

### Example 73: 1-Cyclopropyl-N-[6-cyclopropyl-4-[4-fluoro-2-[4-(fluoromethyl)triazol-1-yl]phenyl]pyridin-2-yl]-5-[(2-methoxyethylamino)methyl]-2-oxopyridine-3-carboxamide

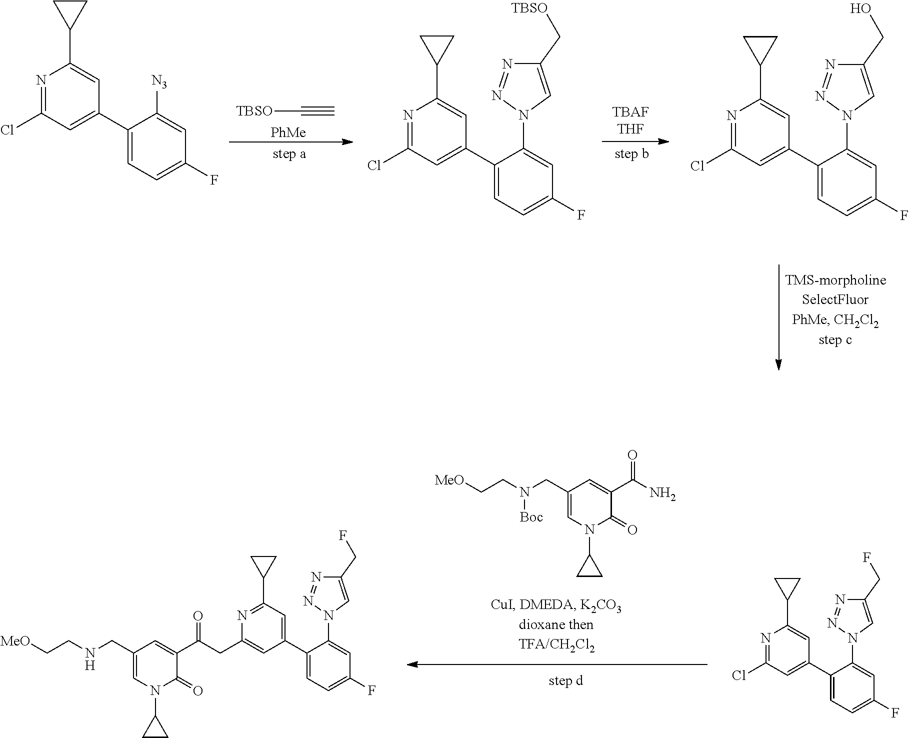

Step a: To a solution of biaryl azide (0.40 g, 1.24 mmol, 1.0 equiv., prepared according to protocol described for Example 73) in toluene (6.2 mL, 0.2 M), t-butyldimethylsilyl propargyl ether (0.32 g, 1.86 mmol, 1.50 equiv.) was added. The reaction vial was flushed with nitrogen, capped and heated to 110° C. for 16 h. The resulting solution was cooled to room temperature and concentrated to dryness. The crude mixture of regioisomers (2:1 favoring corresponding 1,5-disubstituted 1,2,3-triazole) was purified by column chromatography (SiO₂, 0-40% EtOAc gradient in hexanes) to afford the desired 1,5-disubstituted 1,2,3-triazole derivative as a single regioisomer.

Steps b-d were performed according to steps e-g described for Example 73. ¹H NMR (400 MHz, CDCl₃) δ 12.20 (s, 1H), 8.52 (d, J=2.6 Hz, 1H), 8.03-7.94 (m, 1H), 7.78 (d, J=2.6 Hz, 1H), 7.58 (dd, J=8.6, 5.8 Hz, 1H), 7.50 (d, J=2.6 Hz, 1H), 7.42 (dd, J=8.5, 2.6 Hz, 1H), 7.32 (td, J=8.2, 2.6 Hz, 1H), 6.48-6.34 (m, 1H), 5.41 (d, J=48.3 Hz, 2H), 3.80 (s, 2H), 3.56 (t, J=4.9 Hz, 2H), 3.43 (tt, J=7.6, 4.2 Hz, 1H), 3.34 (s, 3H), 2.93 (t, J=5.0 Hz, 2H), 1.80 (tt, J=8.2, 4.8 Hz, 1H), 1.23 (dd, J=14.4, 4.5 Hz, 3H), 1.04-0.74 (m, 6H). ESI MS [M+H]⁺ for C₃₀H₃₁F₂N₇O₃ calcd 575.3, found 575.3.

### Example 74: 1-cyclopropyl-N-[6-cyclopropyl-4-[4-fluoro-2-(4-methylpyridazin-3-yl)phenyl]pyridin-2-yl]-5-[[(2R)-2-methylmorpholin-4-yl]methyl]-2-oxopyridine-3-carboxamide

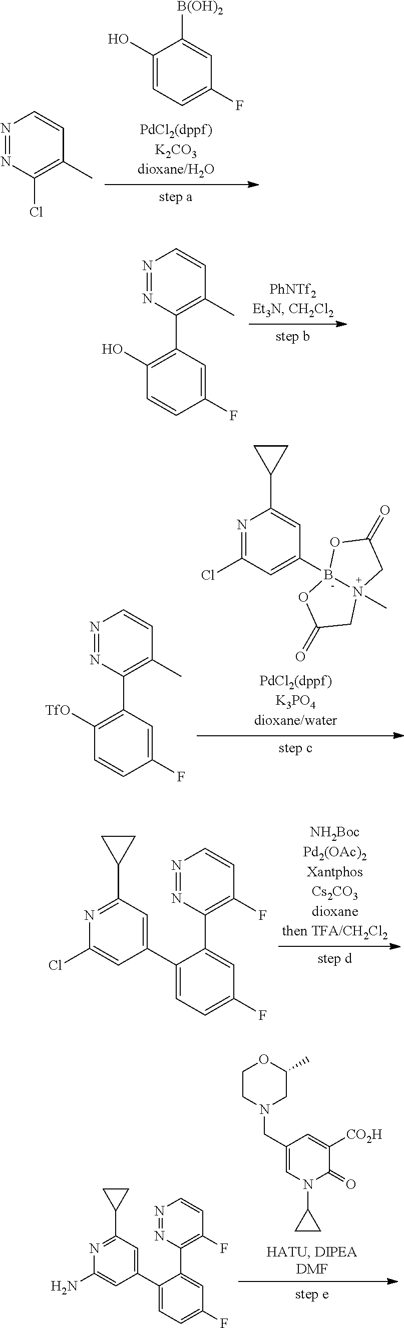

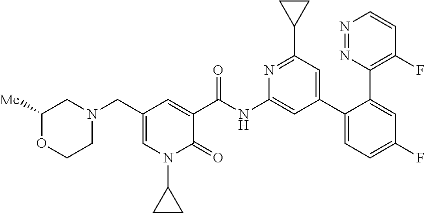 [SMILES: C[C@@H]1CN(Cc2cc(C(=O)Nc3cc(-c4ccc(F)cc4-c4nnccc4F)cc(C4CC4)n3)c(=O)n(C3CC3)c2)CCO1]

Step a: A solution of 3-chloro-4-methylpyridazine (1.3 g, 10.0 mmol, 1 equiv.), 5-fluoro-2-hydroxyphenyl)boronic acid (1.6 g, 10.0 mmol, 1 equiv.) and K₂CO₃ (4.1 g, 30.0 mmol, 3 equiv.) in a dioxane/water mixture (34 mL, 4:1 v/v, 0.3 M) was degassed under vacuum and backfilled with nitrogen (repeated 3 times). Then PdCl₂(dppf) (0.75 g, 1.0 mmol, 0.1 equiv.) was added, and the reaction mixture was heated at 90° C. overnight. The resulting dark solution was cooled to room temperature and diluted with EtOAc (100 mL) and water (100 ml). The organic phase was separated, and the aqueous layer was additionally extracted with EtOAc (2×50 mL). The combined organics were dried over Na₂SO₄ and concentrated to dryness under reduced pressure. The crude product was purified by reversed phase column chromatography (C18, 10-100% CH₃CN in water with 0.1% formic acid) to afford the corresponding biaryl compound.

Step b: To a suspension of the product of step a (1.03 g, 5.0 mmol, 1 equiv.) in dichloromethane (25 mL, 0.2 M) was added PhNTf₂ (2.3 g, 6.6 mmol, 1.3 equiv) followed by triethylamine (2.1 mL, 15.2 mmol, 3 equiv.). The resulting mixture was stirred at room temperature overnight and concentrated to dryness under reduced pressure. The dry residue was directly purified by reversed phase column chromatography (C18, 10-100% CH₃CN in water with 0.1% formic acid) to afford [4-methyl-2-(4-methylpyridazin-3-yl)phenyl]trifluoromethanesulfonate.

Step c: A mixture of the product from step b (1.35 g, 4.0 mmol, 1 equiv.), 1-(2-chloro-6-cyclopropylpyridin-4-yl)-5-methyl-2,8-dioxa-5-azonia-1-boranuidabicyclo[3.3.0]octane-3,7-dione (1.5 g, 4.8 mmol, 1.2 equiv., prepared according to protocol described for Example 15) and K₃PO₄ (1.7 g, 8.0 mmol, 2 equiv.) in dioxane/water (20 mL, 4:1 v/v, 0.2 M) was degassed under vacuum and backfilled with nitrogen (repeated 3 times). PdCl₂(dppf) (0.44 g, 0.6 mmol, 0.15 equiv.) was added. The reaction was heated at 80° C. for 3 h before cooling to room temperature and diluting with EtOAc (50 mL) and brine (50 mL). The organic phase was separated, and the aqueous phase was additionally extracted with EtOAc (25 mL). The combine extract was dried over Na₂SO₄ and the solvent was completely removed under reduced pressure. The residue was purified by reversed phase column chromatography (C18, 10-100% CH₃CN in water with 0.1% formic acid) to afford 4-(2-chloro-6-cyclopropylpyridin-4-yl)-3-(4-methylpyridazin-3-yl)benzonitrile.

Step d: To the solution of 3-[2-(2-chloro-6-cyclopropylpyridin-4-yl)-5-fluorophenyl]-4-methylpyridazine (0.23 g, 0.67 mmol, 1.0 equiv.) in dioxane (6.7 mL, 0.1 M) was added tert-butyl carbamate (87 mg, 0.739 mmol, 1.1 equiv.), Pd(OAc)₂ (15 mg, 0.0672 mmol, 10 mol %), XantPhos (78 mg, 0.13 mmol, 20 mol %) and Cs₂CO₃ (438 mg, 1.344 mmol, 2.0 equiv.). The resulting mixture was heated under N₂ at 100° C. for 1 h. After cooling to room temperature, the mixture was concentrated, and the crude product was purified by column chromatography (SiO₂, 0-100% EtOAc gradient in hexanes) to give tert-butyl N-[6-cyclopropyl-4-[4-fluoro-2-(4-methylpyridazin-3-yl)phenyl]pyridin-2-yl]carbamate. This product (60 mg, 0.143 mmol, 1.0 equiv.) was dissolved in a mixture dichloromethane (1.5 ml) was add TFA (1.5 ml). The resulting solution was stirred at 23° C. for 1 h. All volatiles were removed under reduced pressure to afford 6-cyclopropyl-4-[4-fluoro-2-(4-methylpyridazin-3-yl)phenyl]pyridin-2-amine that was used directly for the next step.

Step e: To a solution of the product from step d (˜0.143 mmol, 1.0 equiv.) and 1-cyclopropyl-5-[[(2R)-2-methylmorpholin-4-yl]methyl]-2-oxopyridine-3-carboxylic acid (42 mg, 0.143 mmol, 1.0 equiv., prepared as described in Example 1) in DMF (1.5 mL, 0.1 M) was added DIPEA (0.10 mL, 0.572 mmol, 4.0 equiv.). The resulting solution was stirred at 23° C. for 30 min before HATU (109 mg, 0.29 mmol, 2.0 equiv.) was added to the above solution. The mixture was stirred for 5 h at room temperature. Upon completion (TLC analysis) the reaction was poured into sat. aq. NaHCO₃ (10 mL) solution, and the product was extracted with CH₂Cl₂ (3×20 mL). The combined organic extract was dried over Na₂SO₄ and concentrated to dryness under reduced pressure. The solvent was removed, and the crude product was purified by reversed phase preparative HPLC (C18, 10-90% CH₃CN gradient in water with 0.1% formic acid) to afford title compound as a white solid. ¹H NMR (400 MHz, DMSO-d₆) δ 12.28 (s, 1H), 8.98 (d, J=5.2 Hz, 1H), 8.31 (d, J=2.6 Hz, 1H), 7.85 (d, J=2.6 Hz, 1H), 7.70-7.60 (m, 2H), 7.53-7.45 (m, 2H), 7.41 (dd, J=9.3, 2.7 Hz, 1H), 6.59 (d, J=1.4 Hz, 1H), 3.69 (d, J=11.7 Hz, 1H), 3.49-3.36 (m, 3H), 2.72-2.53 (m, 3H), 2.33-2.22 (m, 1H), 2.02-1.94 (m, 4H), 1.87 (tt, J=8.3, 4.8 Hz, 1H), 1.67 (t, J=10.6 Hz, 1H), 1.04 (dd, J=7.7, 5.2 Hz, 2H), 1.00 (d, J=6.3 Hz, 3H), 0.92 (dd, J=4.4, 2.5 Hz, 2H), 0.89-0.80 (m, 2H), 0.69 (d, J=4.6 Hz, 2H). ESI MS [M+H]⁺ for C₃₄H₃₆FN₆O₃, calcd 595.3, found 595.3.

### Example 75: N-[4-[4-cyano-2-(3-methylpyridin-2-yl)phenyl]-6-cyclopropylpyridin-2-yl]-1-cyclopropyl-5-[[(2R)-2-methylmorpholin-4-yl]methyl]-2-oxopyridine-3-carboxamide

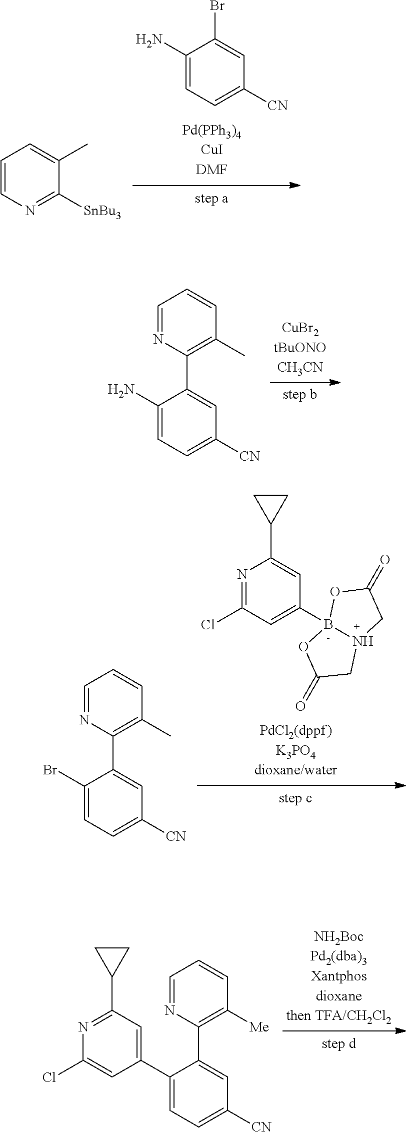

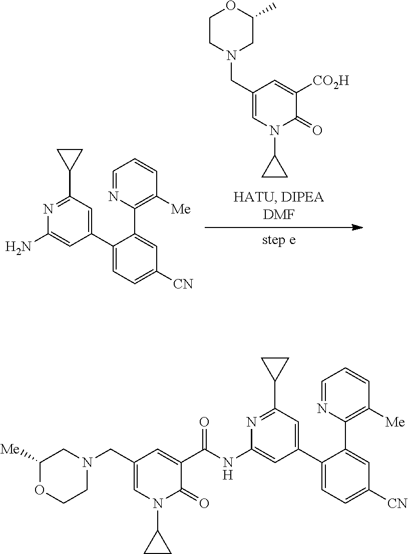 [SMILES: Cc1cccnc1-c1cc(C#N)ccc1-c1cc(N)nc(C2CC2)c1 ; C[C@@H]1CN(Cc2cc(C(=O)O)c(=O)n(C3CC3)c2)CCO1 ; Cc1cccnc1-c1cc(C#N)ccc1-c1cc(NC(=O)c2cc(CN3CCO[C@H](C)C3)cn(C3CC3)c2=O)nc(C2CC2)c1]

Step a: A mixture of tributyl-(3-methylpyridin-2-yl)stannane (5.8 g, 15.2 mmol, 1 equiv.) and 4-amino-3-bromobenzonitrile (3 g, 15.2 mmol, 1 equiv.) in dry DME (76 ml, 0.2 M) was degassed under vacuum and backfilled with nitrogen (repeated 3 times). Then CuI (145 mg, 0.76 mmol, 0.05 equiv.) and Pd(PPh₃)₄ (0.88 g, 0.76 mmol, 0.05 equiv.) were added, and the mixture was stirred at 85° C. overnight. The resulting mixture was cooled to room temperature, diluted with EtOAc (150 mL), filtered through a CELITE® pad, washed with water (2×150 mL), dried over Na₂SO₄ and concentrated to dryness under reduced pressure. The crude product was purified by column chromatography (SiO₂, 0-100% EtOAc in dichloromethane) to afford the desired biaryl coupling product.

Step b: t-BuONO (1.7 mL, 14.4 mmol, 3 equiv.) was added dropwise over 5 min to a 60° C. mixture of the product of step a (1.0 g, 4.8 mmol, 1 equiv.) and CuBr₂ (1.3 g, 5.8 mmol, 1.2 equiv.) in CH₃CN (48 mL, 0.1 M). The resulting dark solution was maintained at 60° C. for 1.5 h, then cooled to room temperature and partitioned between EtOAc (100 mL) and aq. sat. NH₄Cl (50 mL). The mixture was diluted with water (50 mL), the organic phase was separated, and the aqueous phase was additionally extracted with EtOAc (50 mL). The combined organic phase was dried over Na₂SO₄ and concentrated to dryness under reduced pressure. The crude product was purified by column chromatography (SiO₂, 0-80% EtOAc in hexanes) to afford 4-bromo-3-(3-methylpyridin-2-yl)benzonitrile.

Steps c-e were performed similarly to steps c-e of Example 74. ¹H NMR (400 MHz, DMSO-d₆) δ 12.32 (s, 1H), 8.40-8.28 (m, 2H), 8.03 (dd, J=8.1, 1.8 Hz, 1H), 7.91-7.82 (m, 2H), 7.79 (s, 1H), 7.72 (d, J=8.0 Hz, 1H), 7.60-7.50 (m, 1H), 7.22 (dd, J=7.7, 4.8 Hz, 1H), 6.57 (s, 1H), 3.70 (d, J=11.7 Hz, 1H), 3.42 (d, J=8.7 Hz, 3H), 2.73-2.53 (m, 3H), 2.32-2.25 (m, 1H), 2.04-1.78 (m, 5H), 1.73-1.59 (m, 1H), 1.13-0.96 (m, 5H), 0.95-0.89 (m, 2H), 0.89-0.81 (m, 2H), 0.67 (s, 2H). ESI MS [M+H]⁺ for C₃₆H₃₇N₆O₃, calcd 601.3, found 601.3.

### Example 76: 1-cyclopropyl-N-[3-[3-methyl-1-(4-methyl-1,2,4-triazol-3-yl)cyclobutyl]phenyl]-5-[[(3S)-3-methylpiperidin-1-yl]methyl]-2-oxopyridine-3-carboxamide

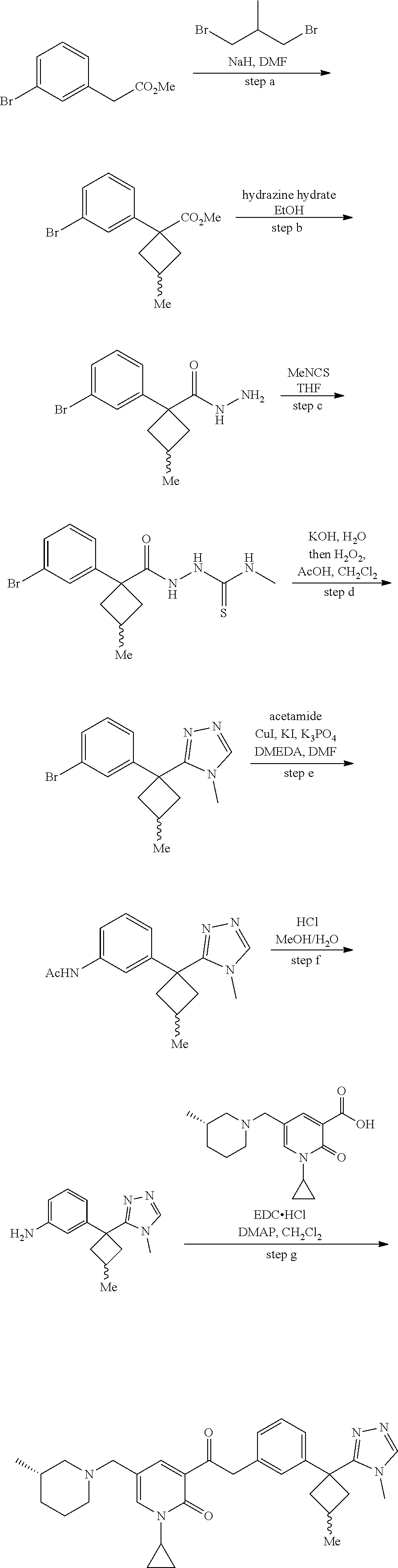

Step a: To a solution of methyl 2-(3-bromophenyl)acetate (10.0 g, 44.05 mmol, 1.0 equiv.) and 1,3-dibromo-2-methylpropane (9.4 g, 44.05 mmol, 1.0 equiv.) in DMF (200 mL, 0.2 M) was added sodium hydride (2.16 g, 88.10 mmol, 2.0 equiv., 60% in mineral oil) at 0° C. The reaction mixture was stirred for 15 min at 0° C. Then the cooling bath was removed and the mixture was stirred overnight at room temperature. The reaction was quenched with aq. sat. NH₄Cl (30 mL), diluted with water (300 mL) and EtOAc (300 mL). The organic phase was separated, and the aqueous phase was additionally extracted with EtOAc (2×100 mL). The combined organic extract was washed with water (2×300 mL) and brine (200 mL), dried over Na₂SO₄ and concentrated to dryness under reduced pressure. The crude product was purified by column chromatography (SiO₂, 0-20% EtOAc gradient in hexane) to yield 1-(3-bromophenyl)-3-methylcyclobutane-1-carboxylate.

Step b: To a solution of the product from step a (8.0 g, 28.4 mmol, 1.0 equiv.) in ethanol (50 mL, 0.5 M) was added hydrazine hydrate (14 ml, 0.28 mol, 10.0 equiv.). The reaction mixture was stirred at 80° C. overnight. Then it was cooled to room temperature and diluted with water (200 mL) and EtOAc (200 mL). The organic phase was separated, and the aqueous phase was extracted with EtOAc (2×50 mL). The combined organic phase was washed with brine (150 mL), dried over Na₂SO₄ and concentrated under reduced pressure to afford 1-(3-bromophenyl)-3-methylcyclobutane-1-carbohydrazide.

Step c: To a solution of the product from step b (8.0 g, 28.2 mmol, 1.0 equiv.) in THF (250 mL, 0.1 M) was added methyl isothiocyanate (6.6 g, 85.1 mmol, 3.0 equiv.). The reaction was stirred at 80° C. for 1 h, then cooled to room temperature, diluted with water (100 mL) and EtOAc (150 mL). The organic phase was separated, and the aqueous phase was extracted with EtOAc (2×50 mL). The combined organic phase was washed with brine (100 mL), dried over Na₂SO₄ and concentrated to afford crude 1-[[1-(3-bromophenyl)-3-methylcyclobutanecarbonyl]amino]-3-methylthiourea that was used for the next step without purification.

Step d: To a solution of the crude urea product from step c (10.0 g, 28.2 mmol, 1.0 equiv.) in H₂O (100 mL, 0.2 M) was added KOH (7.9 g, 0.14 mol, 5.0 equiv.). The resulting solution was stirred at 80° C. for 1 h. Once complete conversion was observed by LC/MS analysis the reaction was cooled to 23° C. and acidified with aq. 1 M HCl to pH˜3 causing product precipitation. The product was collected by vacuum filtration, washed with water (100 mL) and dried on filter under a stream of air. The resulting cyclization product (9.3 g, 27.6 mmol, 1.0 equiv.) was dissolved in dichloromethane (130 mL, 0.2 M) and acetic acid (18 mL) was added. The reaction mixture was cooled to 0° C., and hydrogen peroxide (8.0 mL, 77.6 mmol, 2.8 equiv., 30 wt % in water) was added dropwise over 10 min to maintain a temperature below +10° C. After 20 min the cooling bath was removed, and the reaction was stirred at room temperature for 2 h. Then the resulting solution was diluted with water (200 mL) and dichloromethane (100 mL). The organic phase was separated and sequentially washed with water (2×100 mL) and aq. sat. NaHCO₃ (100 mL). The organic extract was dried over Na₂SO₄ and concentrated to dryness under reduced pressure. The crude mixture was fractionated by column chromatography (SiO₂, 0-10% MeOH in dichloromethane) to yield pure 3-[1-(3-bromophenyl)-3-methylcyclobutyl]-4-methyl-1,2,4-triazole product as an inseparable mixture of diastereomers (3:1 dr).

Step e: To a mixture of 3-[1-(3-bromophenyl)-3-methylcyclobutyl]-4-methyl-1,2,4-triazole (dr=3:1, 0.29 g, 0.94 mmol, 1.0 equiv.) and acetamide (0.23 g, 3.8 mmol, 4.0 equiv.) in DMF (4 mL) was added CuI (36 mg, 0.19 mmol, 0.20 equiv.), KI (110 mg, 0.66 mmol, 0.70 equiv.), K₃PO₄ (0.6 g, 2.8 mmol, 3.0 equiv.) and 1,2-dimethylethylenediamine (66 mg, 0.75 mmol, 0.80 equiv.). The resulting mixture was sparged with nitrogen and heated at 120° C. for 3 h in a sealed vial. It was cooled to room temperature, diluted aq. NH₄Cl (5 mL) and EtOAc (10 mL). The organic phase was separated, and the aqueous phase was additionally extracted with EtOAc (2×10 mL). The combined organic phase was dried over Na₂SO₄ and concentrated to dryness under reduced pressure. The crude product was purified by column chromatography (SiO₂, 0-10% MeOH gradient in dichloromethane) to afford the corresponding coupling product.

Step f: To the solution of the product from step e (50 mg, 0.18 mmol) in MeOH (2 mL) was added aq. HCl (1 mL, 6 M). The resulting mixture was heated at 60° C. overnight, cooled to room temperature and carefully quenched with aq. sat. NaHCO₃ (15 mL). The product was extracted with EtOAc (3×15 mL). The combined organic extract was dried over Na₂SO₄ and concentrated to dryness under reduced pressure to afford crude product that was additionally purified by column chromatography (SiO₂, 0-10% MeOH gradient in dichloromethane).

Step g: To a solution of the product from step f (35 mg, 0.14 mmol, 1.0 equiv.) and 1-cyclopropyl-5-[[(3S)-3-methylpiperidin-1-yl]methyl]-2-oxopyridine-3-carboxylic acid (61.0 mg, 0.21 mmol, 1.5 equiv., obtained according to the protocol described for Example 1) in dichloromethane (2 mL) was added EDC HCl (48 mg, 0.25 mmol, 1.8 equiv.) and DMAP (5 mg, 0.042 mmol, 0.2 equiv.). The resulting mixture was stirred at room temperature overnight, diluted with water (10 mL), and the product was extracted with dichloromethane (3×10 mL). The combined organic extract was dried over Na₂SO₄ and concentrated to dryness under reduced pressure. The crude product was purified by reversed phase preparative HPLC (C18, 10-90% CH₃CN gradient in water with 0.1% formic acid) to afford title compound as a mixture of inseparable diastereomers (dr=3:1). ¹H NMR (400 MHz, DMSO-d₆, major diastereomer) δ 12.23 (s, 1H), 8.41-8.27 (m, 2H), 7.84 (d, J=2.6 Hz, 1H), 7.65 (dd, J=8.0, 2.0 Hz, 1H), 7.59 (t, J=2.0 Hz, 1H), 7.48-7.29 (m, 1H), 7.07 (d, J=7.9 Hz, 0.78H), 6.93 (d, J=7.9 Hz, 0.22H), 3.47 (tt, J=7.7, 4.3 Hz, 1H), 3.30 (s, 2H), 3.21 (s, 0.7H), 3.17 (s, 2.3H), 3.13-2.64 (m, 4H), 2.60-2.49 (m, 2H), 2.40-2.15 (m, 1H), 1.92-1.77 (m, 1H), 1.72-1.34 (m, 5H), 1.16-1.00 (m, 5H), 1.02-0.91 (m, 2H), 0.88-0.74 (m, 4H). ESI MS [M+H]⁺ for C₃₀H₃₈N₆O₂, calcd 515.3, found 515.3.

### Example 77: N-[3-[3-methyl-1-(4-methyl-1,2,4-triazol-3-yl)cyclobutyl]phenyl]-5-[[(3S)-3-methylpiperidin-1-yl]methyl]-2-oxo-1-prop-2-enylpyridine-3-carboxamide

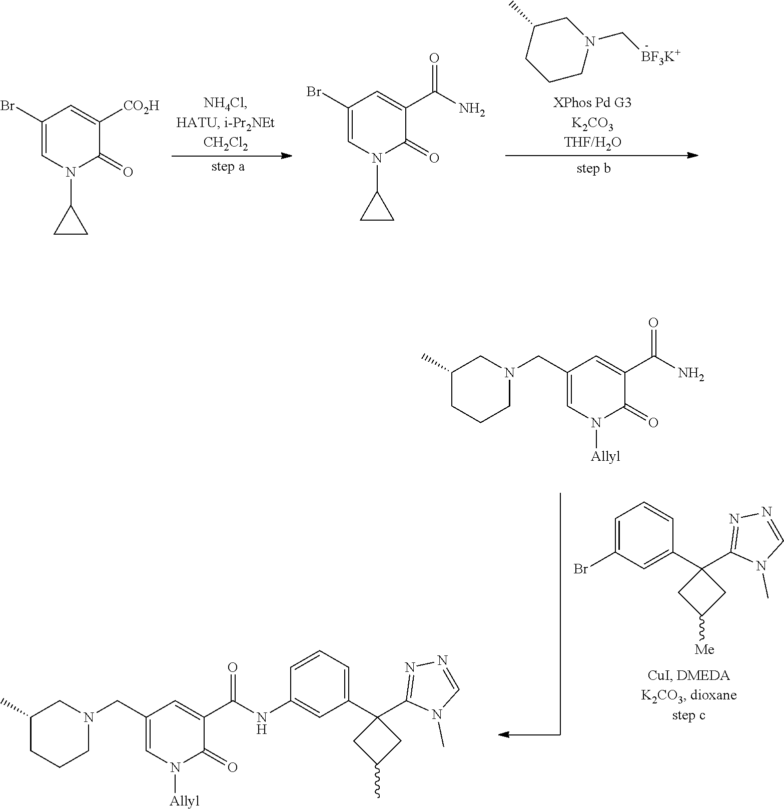

Step a: To mixture of 5-bromo-1-cyclopropyl-2-oxopyridine-3-carboxylic acid (0.3 g, 1.16 mmol, 1.0 equiv.), ammonium chloride (0.1 g, 1.74 mmol, 1.5 equiv.), HATU (0.7 g, 1.7 mmol, 1.5 equiv.) and i-Pr₂NEt (0.6 mL, 3.5 mmol, 3.0 equiv.) in dichloromethane (5 mL, 0.2 M) was stirred overnight at room temperature. The resulting suspension was partitioned between water (15 mL) and dichloromethane (15 mL), the organic phase was separated, and the aqueous phase was additionally extracted with dichloromethane (10 mL). The combined organic extract was dried over Na₂SO₄ and concentrated to dryness under reduced pressure to afford the crude amide product that was purified by column chromatography (SiO₂, 0-20% MeOH in dichloromethane) to yield 5-bromo-1-cyclopropyl-2-oxopyridine-3-carboxamide.

Step b: To the product of step a (0.32 g, 1.3 mmol, 1.0 equiv.) in a dioxane/water mixture (13 mL, 9:1 v/v, 0.1 M), potassium trifluoro-{[(3S)-3-methylcyclohexyl]methyl}borate (0.34 g, 1.9 mmol, 1.5 equiv.), XPhos Pd G3 (53 mg, 0.06 mmol, 0.05 equiv.), XPhos (30 mg, 0.06 mmol, 0.05 equiv) and K₂CO₃ (520 mg, 3.774 mmol, 3.0 equiv.) were added. The resulting mixture was purged with nitrogen for 5 min, then heated at 80° C. overnight under nitrogen atmosphere. The reaction mixture was cooled to room temperature and partitioned between EtOAc (20 mL) and water (10 mL). The organic phase was separated, dried over Na₂SO₄ and concentrated to dryness under reduced pressure. The crude product was purified by column chromatography (SiO₂, 0-10% MeOH gradient in dichloromethane) to afford the 5-[[(3S)-3-methylpiperidin-1-yl]methyl]-2-oxo-1-prop-2-enylpyridine-3-carboxamide.

Step c: The title compound was prepared as a mixture of diastereomers (dr=3:1) in a similar fashion to step f of Example 5 using 3-[1-(3-bromophenyl)-3-methylcyclobutyl]-4-methyl-1,2,4-triazole that was prepared according to Example 77. ¹H NMR (400 MHz, CDCl₃, major diastereomer) δ 12.10 (s, 1H), 8.60 (d, J=2.7 Hz, 1H), 7.97 (t, J=2.0 Hz, 1H), 7.94 (s, 1H), 7.61 (dt, J=8.1, 1.2 Hz, 1H), 7.52 (d, J=2.6 Hz, 1H), 7.29 (d, J=7.9 Hz, 1H), 6.94 (dt, J=7.8, 1.3 Hz, 1H), 6.12-5.90 (m, 1H), 5.35 (dd, J=10.3, 1.1 Hz, 1H), 5.22 (dd, J=17.0, 1.3 Hz, 1H), 4.70 (dt, J=5.8, 1.6 Hz, 2H), 3.32 (s, 2H), 3.19 (s, 3H), 2.87 (q, J=5.1, 4.2 Hz, 2H), 2.74 (dd, J=19.1, 9.5 Hz, 1H), 2.68-2.56 (m, 2H), 1.91 (td, J=11.2, 2.9 Hz, 1H), 1.59-1.47 (m, 8H), 1.13 (d, J=5.8 Hz, 3H), 0.85 (d, J=5.8 Hz, 3H). ESI MS [M+H]⁺ for C₃₀H₃₉N₆O₂, calcd 580.1, found 580.1.

### Example 78: 1-(cyclopropylmethyl)-N-[3-[3-methyl-1-(4-methyl-1,2,4-triazol-3-yl)cyclobutyl]phenyl]-5-[[(3S)-3-methylpiperidin-1-yl]methyl]-2-oxopyridine-3-carboxamide

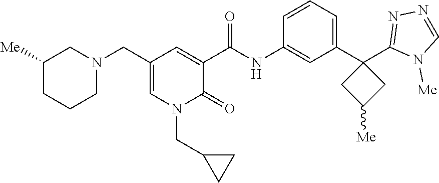 [SMILES: CC1CC(c2cccc(NC(=O)c3cc(CN4CCC[C@H](C)C4)cn(CC4CC4)c3=O)c2)(c2nncn2C)C1]

The title compound was prepared in a similar fashion to that described for Example 78 as a mixture of diastereomers (dr=3:2) using the product of step a, Example 56 as the starting material. ¹H NMR (400 MHz, DMSO-d₆, major diastereomer) δ 12.17 (s, 1H), 8.33 (s, 1H), 8.30-8.20 (m, 1H), 7.99 (s, 1H), 7.60-7.36 (m, 2H), 7.28 (q, J=9.0, 8.5 Hz, 1H), 7.04-6.82 (m, 1H), 3.87 (d, J=7.3 Hz, 2H), 3.16-3.07 (m, 3H), 3.01 (t, J=8.9 Hz, 1H), 2.79-2.55 (m, 4H), 2.50-2.46 (m, 2H), 2.26 (s, 1H), 2.15 (t, J=10.0 Hz, 1H), 1.79 (t, J=11.2 Hz, 1H), 1.62-1.44 (m, 4H), 1.44-1.29 (m, 1H), 1.30-1.18 (m, 1H), 1.02 (t, J=5.4 Hz, 3H), 0.75 (d, J=5.2 Hz, 4H), 0.51-0.32 (m, 4H), ESI MS [M+H]⁺ for C₃₁H₄₁N₆O₂, calcd 529.3, found 529.2.

### Example 79: 1-cyclopropyl-N-[3-[3,3-difluoro-1-(4-methyl-1,2,4-triazol-3-yl)cyclobutyl]phenyl]-5-[[(3S)-3-methylpiperidin-1-yl]methyl]-2-oxopyridine-3-carboxamide

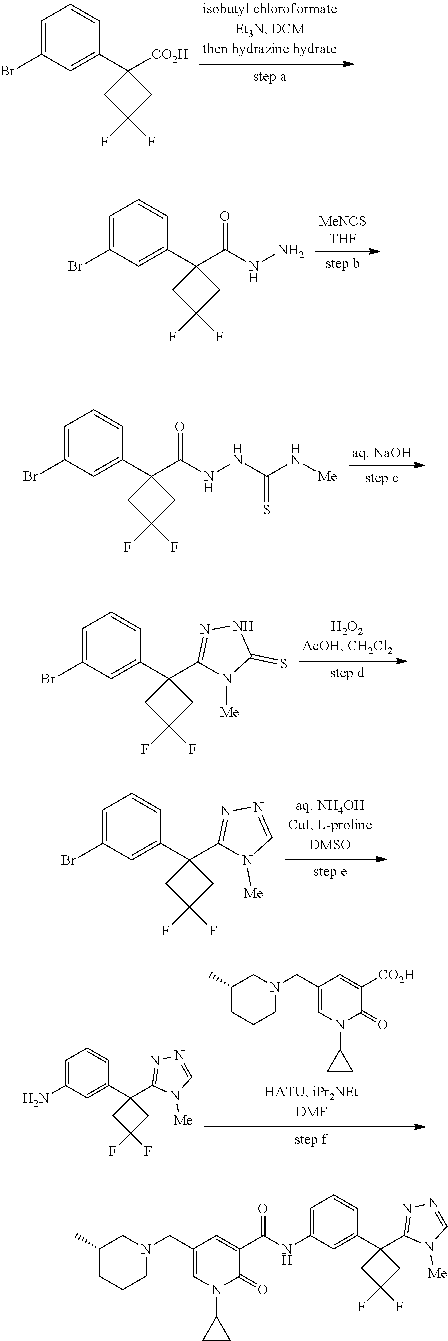

Step a: Triethylamine (1.1 mL, 7.9 mmol) was added to a solution of 1-(3-bromophenyl)-3,3-difluorocyclobutane-1-carboxylic acid (1.0 g, 3.4 mmol) in dichloromethane (35 mL). The resulting solution was placed in 100 mL single neck round bottom flask equipped with magnetic stirring bar and drying tube. The reaction was cooled to 0° C. before isobutyl chloroformate (0.49 mL, 3.8 mmol) was added dropwise over 1 min. The reaction mixture was allowed to warm and stirred for 1 h at 23° C. The solution was cooled back to 0° C. and hydrazine hydrate (0.8 mL, 13.8 mmol, 55% purity) was added in one portion. The reaction mixture was allowed to warm to 23° C. and stirred for 20 min. Then it was diluted with dichloromethane (20 mL) and poured into aq. sat. sodium bicarbonate solution (20 mL). The organic phase was separated, and the aqueous phase was additionally extracted with dichloromethane (2×15 mL). The combined organic extract was dried over sodium sulfate, and all volatiles were removed under reduced pressure. The crude product obtained upon concentration was used directly for step b.

Step b: The acylhydrazine obtained in step a (3.4 mmol) was dissolved in THF and methyl isothiocyanate (0.7 mL, 10.3 mmol) was added. The resulting mixture was maintained at 65° C. for 1 h. Then it was cooled to room temperature, and all volatiles were removed under reduced pressure. The crude product was purified by column chromatography (SiO₂, dichloromethane/EtOAc gradient) to produce the desired thiosemicarbazide.

Step c: Thiosemicarbazide (1.2 g, 3.3 mmol) from step b was dissolved in 16 mL of 1 M aq. NaOH and the resulting clear solution was stirred at 70° C. for 30 min. The reaction mixture was cooled to room temperature, acidified with 1 M aq. hydrochloric acid until pH˜3, and the formed product was extracted with EtOAc (3×25 mL). The combined organic extract was dried over sodium sulfate and concentrated to dryness to produce the cyclized product that was directly used for the next step.

Step d: The cyclized product of step c (3.3 mmol) was dissolved in a mixture of dichloromethane (14.5 mL) and acetic acid (1.8 mL) and placed in a 100 mL single neck round bottom flask equipped with magnetic stirring bar. The reaction mixture was cooled to 0° C., and hydrogen peroxide (0.9 mL, 8.5 mmol, 30% aq. solution) was added. The resulting biphasic mixture was stirred for 20 min, then cooling bath was removed, and the reaction mixture was maintained at room temperature for an additional 1 h. The mixture was diluted with dichloromethane (30 mL) and 1 M aq. NaOH (30 mL). The organic phase was separated, and the aqueous phase was additionally extracted with dichloromethane (2×20 mL). The combined organic extract was washed with 1 M aq. NaOH (40 mL), water (40 mL) and brine (40 mL), dried over sodium sulfate and concentrated to dryness under reduced pressure to produce the desired 1,3,4-triazole.

Step e: A mixture of the product from step d (0.3 g, 0.9 mmol, 1 equiv.), L-proline (42 mg, 0.36 mmol, 0.4 equiv.) and DMSO (1.8 mL, 0.5 M) was carefully degassed under vacuum and backfilled with nitrogen (repeated 3 times), then CuI (35 mg, 0.18 mmol, 0.2 equiv.) was added. The resulting mixture was stirred at room temperature for 15 min before aq. conc. NH₄OH (0.3 mL) was added. The reaction mixture was stirred at 80° C. overnight, then cooled to room temperature and partitioned between water (15 mL) and EtOAc (15 mL). The organic phase was separated, and the aqueous phase was additionally extracted with EtOAc (2×15 mL). The combined organic extract was washed with water (30 mL) and brine (30 mL), dried over Na₂SO₄ and concentrated to dryness under reduced pressure. The crude residue was fractionated by reversed phase column chromatography (C18, 10-100% CH₃CN in water, 0.1% formic acid) to yield the desired product.

Step f: The reaction was performed according to the protocol described for step e of Example 75. ¹H NMR (400 MHz, CDCl₃) δ 12.18 (s, 1H), 8.55 (d, J=2.6 Hz, 1H), 8.03 (s, 1H), 7.90 (t, J=2.0 Hz, 1H), 7.71-7.54 (m, 2H), 7.32 (t, J=8.0 Hz, 1H), 6.92 (ddd, J=7.8, 2.0, 1.0 Hz, 1H), 3.72 (dt, J=14.5, 12.1 Hz, 2H), 3.46-3.23 (m, 8H), 2.75 (dd, J=19.2, 9.7 Hz, 2H), 1.97-1.87 (m, 1H), 1.79-1.47 (m, 5H), 1.32-1.21 (m, 2H), 1.02-0.95 (m, 2H), 0.94-0.81 (m, 4H). ESI MS [M+H]⁺ for C₂₉H₃₄F₂N₆O₂, calcd 537.3, found 537.3.

### Example 80: 1-cyclopropyl-N-[3-[3-methoxy-1-(4-methyl-1,2,4-triazol-3-yl)cyclobutyl]phenyl]-5-[[(3S)-3-methylpiperidin-1-yl]methyl]-2-oxopyridine-3-carboxamide

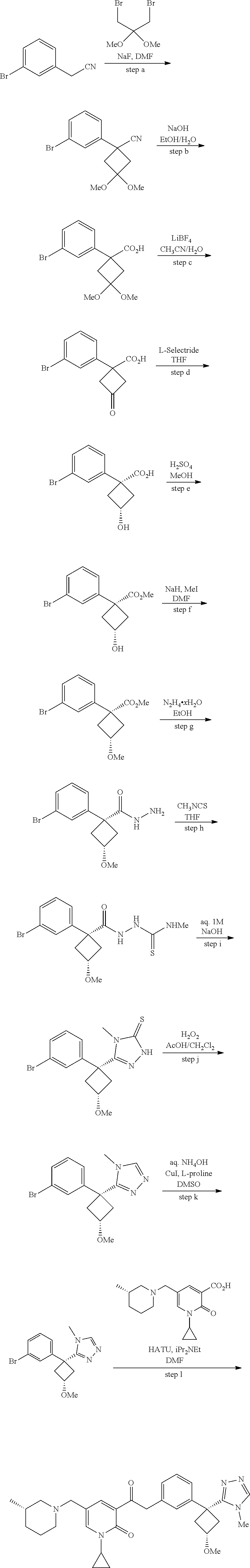

Step a: Sodium hydride (2.3 g, 57.0 mmol, 2.2 equiv.) was suspended in dry DMF (50 mL), and the resulting mixture was placed in 100 mL round bottom flask equipped with a magnetic stirring bar and reflux condenser with a drying tube. 2-(3-Bromophenyl)acetonitrile (5.0 g, 26.0 mmol, 1 equiv.) was added to the reaction mixture dropwise over 20 min at 0° C. After stirring for 20 min 1,3-dibromo-2,2-dimethoxypropane (6.7 g, 26 mmol, 1 equiv.) was added. The cooling bath was removed, and the suspension was heated at 65° C. overnight. The resulting solution was carefully poured in aq. sat. NH₄Cl (100 mL). The biphasic mixture was diluted with ethyl acetate (100 mL) and water (100 mL). The organic phase was separated, and the aqueous phase was additionally extracted with ethyl acetate (2×50 mL). The combined organic extract was washed with water (2×70 mL) and brine (100 mL), dried over sodium sulfate and concentrated to dryness under reduced pressure. The crude product was purified by column chromatography (SiO₂, hexanes/EtOAc gradient) to produce the desired nitrile.

Step b: The nitrile from step a (3.5 g, 12.0 mmol, 1 equiv.) was refluxed overnight in a mixture of ethanol (20 mL) and water (20 mL) containing NaOH (4.8 g, 0.12 mol, 10 equiv.). The mixture was cooled to room temperature and EtOH was removed under reduced pressure. The resulting aqueous solution was diluted with water (50 mL), extracted with diethyl ether (3×50 mL) and acidified with 1 M aq. hydrochloric acid to pH˜1. The product was extracted with ethyl acetate (3×50 mL). The combined organic extract was dried over sodium sulfate and concentrated to dryness under reduced pressure to provide the corresponding carboxylic acid.

Step c: A mixture of acid from step b (1.0 g, 3.2 mmol, 1 equiv.) and LiBF₄ (0.33 g, 3.5 mmol, 1.1 equiv.) was dissolved in acetonitrile (10 mL) containing water (0.2 mL), and the solution was stirred at 60° C. for 1 h. The reaction was cooled to room temperature and diluted with water (30 mL) and ethyl acetate (20 mL). The organic phase was separated, additionally washed with water (30 mL) and brine (15 mL), dried over sodium sulfate and concentrated to dryness under reduced pressure to yield the corresponding γ-ketoacid.

Step d: A solution of γ-ketoacid from step c (0.5 g, 1.9 mmol, 1 equiv.) in THF (3 mL) was added to a solution of L-Selectride® (4.2 ml, 4.1 mmol, 2.2 equiv., 1 M solution in THF) preheated to 50° C. The reaction mixture was maintained at 50° C. for 2 h before it was cooled to room temperature. The reaction mixture was diluted with EtOAc (30.0 mL) and washed with 1 M aq. hydrochloric acid (15 mL) and brine (15 mL). The organic phase was dried over sodium sulfate and concentrated to dryness to produce the desired γ-hydroxyacid as cis-isomer (dr=20:1).

Step e: γ-Hydroxyacid from step d (1.0 g, 3.7 mmol, 1 equiv.) was dissolved in MeOH (18.0 mL), concentrated sulfuric acid (0.1 mL) was added, and the reaction mixture was refluxed for 1 h. The resulting solution was cooled to room temperature and concentrated to ˜3.0 mL under reduced pressure. The concentrate was diluted with water (25 mL) and ethyl acetate (25 mL), the organic phase was separated, and the aqueous phase was additionally extracted with EtOAc (2×10 mL). The combined organic extract was washed with aq. sat. sodium bicarbonate (10 mL) and brine (10 mL), dried over sodium sulfate and concentrated to dryness under reduced pressure to provide cis-methyl 1-(3-bromophenyl)-3-hydroxycyclobutane-1-carboxylate.

Step f: Methyl iodide (0.26 mL, 4.25 mmol, 1.1 equiv.) was added to a suspension of cis-methyl 1-(3-bromophenyl)-3-hydroxycyclobutane-1-carboxylate (1.10 g, 3.86 mmol, 1 equiv.) and NaH (0.17 g, 4.25 mmol, 1 equiv.) in DMF (7.7 mL) at 0° C. The reaction was warmed up to room temperature, stirred for 2 h, then quenched with methanol, and diluted with EtOAc. The mixture was washed with water (3×5 mL), then brine, and the combined organic phase was dried over Na₂SO₄ and concentrated to dryness. The crude material was purified by flash chromatography (SiO₂, 0-30% EtOAc gradient in hexanes) to give the desired alkylation product.

Step g: To a solution of product from step f (0.76 g, 2.5 mmol, 1 equiv.) in EtOH (5.0 mL, 0.5 M) was added hydrazine hydrate (1.20 mL, 24.0 mmol, 10 equiv.) at ambient temperature. The resulting mixture was stirred for 16 h at 80° C. The mixture was cooled down to room temperature and evaporated under vacuum. The residue was diluted with water and extracted with EtOAc (3×5 mL). The combined organic phase was washed with brine, dried over Na₂SO₄, concentrated and the dry residue was used in the next step without further purification.

Step h: The product from step g (˜2.5 mmol, 1 equiv.) was dissolved in THF (16 mL), and methylisothiocyanate (0.51 mL, 7.5 mmol, 3 equiv.) was added. The reaction was stirred for 2 h at 65° C. Upon completion, the reaction was concentrated to dryness. The dry residue was triturated with MTBE (20 mL), and the formed precipitate was collected by filtration, dried under vacuum and used for the next step without purification.

Step i: The product from step h (0.80 g, 2.1 mmol, 1 equiv.) was treated with aq. 1 M NaOH solution (12 mL) for 30 min at 65° C. The reaction mixture was cooled to room temperature, acidified to pH˜1 with 1 M HCl and extracted with EtOAc (2×15 mL). The combined organic phase was washed with brine, dried over Na₂SO₄ and concentrated to dryness under reduced pressure. The crude residue product was used for the next step without further purification.

Step j: Acetic acid (1.2 mL) and hydrogen peroxide (0.20 mL, 6.3 mmol, 3 equiv, 30 wt % in water) were sequentially added to a solution of the product from step i (˜2.1 mmol, 1 equiv.) in CH₂Cl₂ (8 mL) at 0° C. The reaction mixture was stirred for 30 min at 0° C., then cooling bath was removed, and the resulting biphasic solution was stirred overnight at room temperature. The mixture was diluted with dichloromethane (25 mL) and basified by addition of aq. 1 M NaOH to pH˜12. The organic phase was separated, and the aqueous phase was additionally extracted with dichloromethane (2×10 mL). The combined organic extract was washed with water (20 mL) and brine (20 mL), dried over Na₂SO₄ and concentrated to dryness under reduced pressure. The crude material was purified by reversed phase column chromatography (C18, 10-100% CH₃CN in water with 0.1% formic acid) to afford cis-3-[1-(3-bromophenyl)-3-methoxycyclobutyl]-4-methyl-1,2,4-triazole.

Steps k-l were performed according to protocols described for step e of Example 80 and step e of Example 75, respectively. ¹H NMR (400 MHz, CDCl₃) δ 12.15 (s, 1H), 8.57 (d, J=2.6 Hz, 1H), 7.95 (d, J=1.7 Hz, 2H), 7.65-7.51 (m, 2H), 7.39-7.27 (m, 1H), 6.94 (ddd, J=7.9, 2.0, 1.0 Hz, 1H), 4.16 (p, J=7.4 Hz, 1H), 3.48-3.40 (m, 1H), 3.32-3.28 (m, 2H), 3.26 (s, 3H), 3.24 (s, 3H), 3.14-3.07 (m, 2H), 2.99-2.92 (m, 2H), 2.74 (dd, J=18.8, 9.8 Hz, 2H), 1.95-1.85 (m, 1H), 1.75-1.48 (m, 5H), 1.31-1.20 (m, 2H), 1.02-0.95 (m, 2H), 0.93-0.81 (m, 4H).

### Example 81: 1-cyclopropyl-N-[3-[3-methoxy-1-(4-methyl-1,2,4-triazol-3-yl)cyclobutyl]phenyl]-5-[[(3S)-3-methylpiperidin-1-yl]methyl]-2-oxopyridine-3-carboxamide

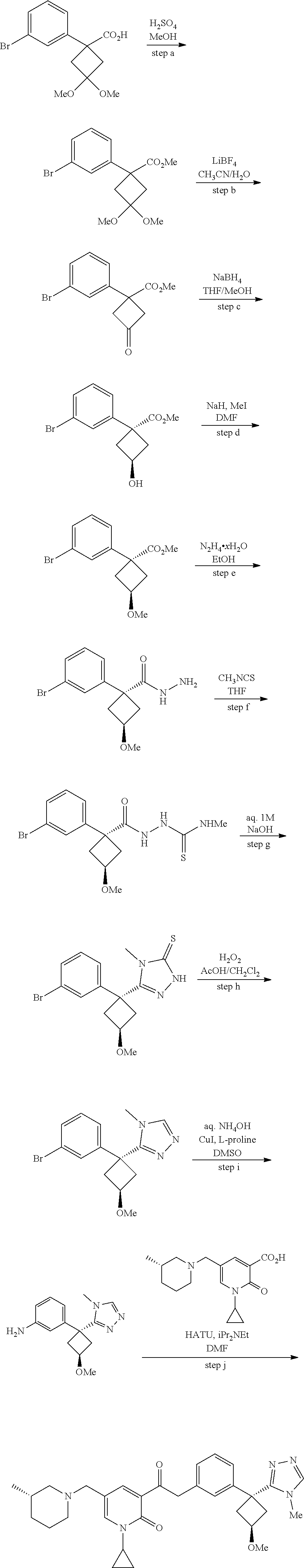

Step a: 1-(3-Bromophenyl)-3,3-dimethoxycyclobutane-1-carboxylic acid (6.2 g, 20.0 mmol, 1 equiv.) was dissolved in MeOH (100 mL, 0.2 M), concentrated sulfuric acid (3.0 mL) was added, and the reaction mixture was refluxed for 1 h. The resulting solution was cooled to room temperature and concentrated to ˜10 mL under reduced pressure. The concentrate was diluted with water (100 mL) and ethyl acetate (100 mL), the organic phase was separated, and the aqueous phase was additionally extracted with EtOAc (2×30 mL). The combined organic extract was washed with aq. sat. sodium bicarbonate (100 mL) and brine (100 mL), dried over sodium sulfate and concentrated to dryness under reduced pressure to provide the corresponding ester that was used for the next step without purification.

Step b: Ester from step a (˜20.0 mmol, 1 equiv.) and LiBF₄ (2.0 g, 21.9 mmol, 1.1 equiv.) were dissolved in acetonitrile (63 mL) and water (1.30 mL) mixture, and the solution was stirred at 60° C. for 1 h. The reaction was cooled to room temperature and diluted with water (150 mL) and ethyl acetate (100 mL). The organic phase was separated, additionally washed with water (100 mL) and brine (50 mL), dried over sodium sulfate and concentrated to dryness under reduced pressure to yield the corresponding γ-ketoester that was used for the next step without purification.

Step c: γ-Ketoester from step b (0.5 g, 1.8 mmol, 1 equiv.) was dissolved in a mixture of THF (9.0 mL) and MeOH (9.0 mL). The reaction mixture was cooled to −78° C. and a solution of NaBH₄ (134 mg, 3.5 mmol, 2 equiv.) in MeOH (2.0 mL) was added dropwise over 1 min. The resulting solution was maintained at −78° C. for 2 h. Once TLC analysis indicated complete transformation the reaction was diluted with ethyl acetate (30 mL) and 1 M aq. HCl (15 mL). The organic phase was separated, and the aqueous phase was extracted with EtOAc (2×15 mL). The combined organic extract was washed with brine (30 mL), dried over sodium sulfate and concentrated to dryness under reduced pressure. The crude product was purified by column chromatography (SiO₂, hexanes/EtOAc gradient) to produce the desired trans-methyl 1-(3-bromophenyl)-3-hydroxycyclobutane-1-carboxylate (dr=20:1).

Steps d-j were performed according to protocols described for steps f-l of Example 81. ¹H NMR (400 MHz, CDCl₃) δ 12.11 (s, 1H), 8.55 (d, J=2.6 Hz, 1H), 8.02 (s, 1H), 7.72-7.64 (m, 2H), 7.59 (br. s, 1H), 7.33-7.18 (m, 1H), 6.85 (dt, J=7.8, 1.2 Hz, 1H), 4.06 (p, J=7.3 Hz, 1H), 3.43 (tt, J=7.7, 4.3 Hz, 1H), 3.38-3.29 (m, 4H), 3.27 (s, 3H), 3.23 (s, 3H), 2.75 (dd, J=19.1, 9.7 Hz, 2H), 2.62 (ddd, J=10.8, 6.7, 2.7 Hz, 2H), 1.91 (t, J=11.1 Hz, 2H), 1.78-1.43 (m, 5H), 1.35-1.18 (m, J=6.9 Hz, 2H), 1.08-0.95 (m, 2H), 0.94-0.78 (m, 1H).

### Example 82: N-[4-[4-chloro-2-(3,3-difluorocyclobutanecarbonyl)phenyl]-6-(2,2,2-trifluoroethoxy)pyridin-2-yl]-1-cyclopropyl-5-[[[(2S)-2-methoxypropyl]amino]methyl]-2-oxopyridine-3-carboxamide

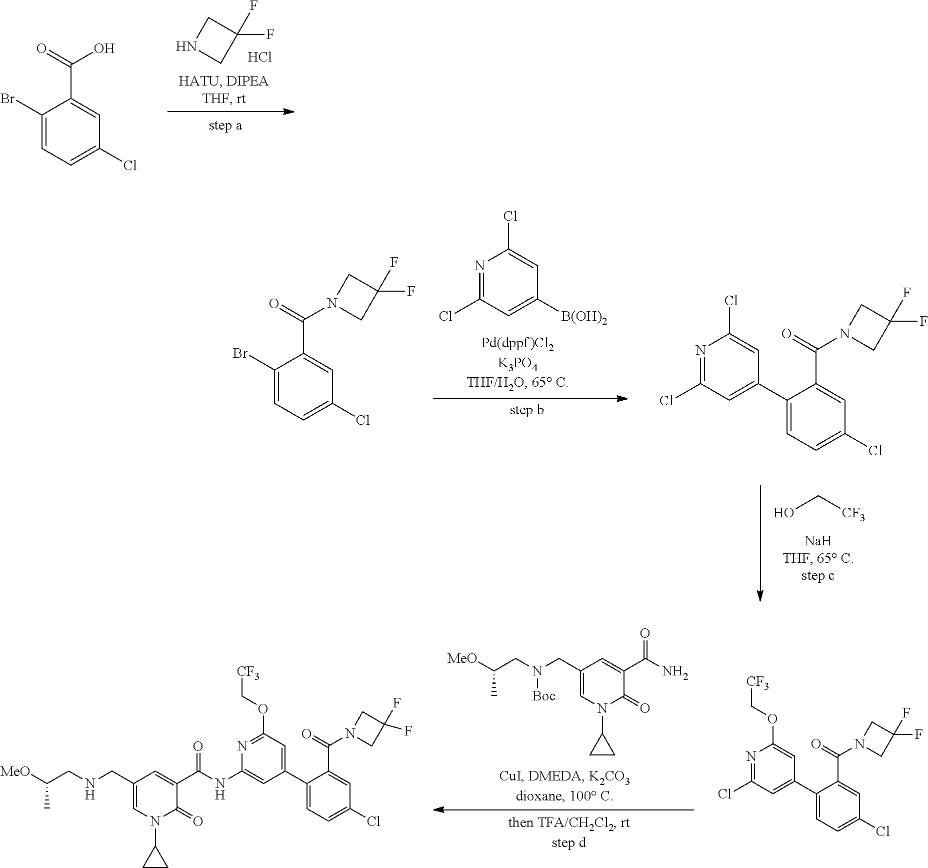

Step a: To a mixture of 2-bromo-5-chlorobenzoic acid (8.0 g, 0.034 mol, 1.0 equiv.), 3,3-difluorozetidine hydrochloride (5.30 g, 0.041 mol, 1.20 equiv.) and Hunig's base (17.8 mL, 0.10 mol, 3.0 equiv.) in THF (85 mL, 0.4 M) was added HATU (15.6 g, 0.041 mmol, 1.20 equiv.). The resulting mixture was stirred at room temperature for 16 h. Once cooled down to the room temperature, the mixture was poured into 500 mL of water. After 20 min, the precipitate was filtered and washed with water (2×100 mL), then with a mixture of Et₂O/hexanes (1:1, 30 mL) to give desired product.

Step b: A suspension of the product from step a (5.0 g, 0.016 mmol, 1.0 equiv.), (2,6-dichloropyridin-4-yl)boronic acid (3.10 g, 0.016 mol, 1.05 equiv.) and K₃PO₄ (6.80 g, 0.032 mol, 2.0 equiv.) in THF/water mixture (80 mL, 3:1 v/v, 0.2 M) was purged with N₂ for 10 minutes. Pd(dppf)Cl₂ (0.59 g, 0.80 mmol, 0.05 equiv.) was added, and the reaction was heated to 60° C. for 1 h. Then it was cooled to room temperature, diluted with a ˜1:1 mixture of saturated aqueous NaCl/water (50 mL) and extracted with EtOAc (2×40 mL). The combined organics were dried over Na₂SO₄, filtered, and concentrated under vacuum. The crude residue was purified via column chromatography (SiO₂, EtOAc in hexanes, 0 to 40%) to afford the desired biaryl product.

Step c: To a solution of 2,2,2-trifluoroethanol (0.16 mL, 2.22 mmol, 1.20 equiv.) in THF (9.20 mL, 0.2 M) was added NaH (90 mg, 60% in mineral oil, 2.22 mmol, 1.20 equiv.) at 0° C. The cooling bath was removed, and the mixture was stirred at the room temperature for 10 minutes, until all NaH was dissolved. The product from step b (700 mg, 1.85 mmol, 1.0 equiv.) was added to the reaction mixture and stirred for 12 h at 65° C. Then, the reaction mixture was quenched with water (4.0 mL), diluted with EtOAc (8.0 mL), washed with sat. aq. NH₄Cl (5.0 ml), and then water (5.0 mL). The organic phase was dried over Na₂SO₄, concentrated and the crude residue was purified by column chromatography (SiO₂, EtOAc in hexanes, 0 to 20%) to give desired product.

Step d: A solution of the product from step c (81 mg, 0.18 mmol, 1.0 equiv.), tert-butyl N-[(5-carbamoyl-1-cyclopropyl-6-oxopyridin-3-yl)methyl]-N-[(2S)-2-methoxypropyl]carbamate (70 mg, 0.18 mmol, 1.0 equiv., prepared according to example 89) and K₂CO₃ (152 mg, 1.10 mmol, 6.0 equiv.) in dioxane (3.70 ml, 0.05 M) was degassed with a stream of bubbling nitrogen for ten minutes. CuI (70 mg, 0.36 mmol, 2.0 equiv.) and DMEDA (82 μL, 0.36 mmol, 2.0 equiv.) were added, and the reaction was heated for 16 h at 100° C. Then the mixture was cooled to room temperature, diluted with aq. NH₄Cl (2 mL) and extracted with EtOAc (2×5 mL). The combined organic phase was washed with water (2×4 mL) and brine (4 mL), dried over Na₂SO₄, and concentrated to dryness. The crude residue was purified by reversed-phase column chromatography (C18, 10-100% CH₃CN in water with 0.1% formic acid) to afford corresponding coupling product. The residual material was then treated with TFA/DCM (v/v 1:10, 2 mL) at room temperature for 1 h. The mixture was concentrated under vacuum, and the crude residue was then purified by reversed-phase column chromatography (C18, 10-100% CH₃CN in water with 0.1% formic acid) to afford the title compound. ¹H NMR (400 MHz, CDCl₃) δ 12.38 (s, 1H), 8.57 (d, J=2.7 Hz, 1H), 8.17 (d, J=1.3 Hz, 1H), 7.69 (d, J=2.6 Hz, 1H), 7.50 (d, J=1.8 Hz, 3H), 6.69 (d, J=1.3 Hz, 1H), 4.80 (q, J=8.6 Hz, 2H), 4.37 (t, J=11.9 Hz, 2H), 4.06 (t, J=11.5 Hz, 2H), 3.67 (d, J=2.2 Hz, 2H), 3.56-3.41 (m, 2H), 3.35 (s, 3H), 2.74-2.54 (m, 2H), 2.03 (s, 2H), 1.38-1.22 (m, 2H), 1.15 (d, J=6.1 Hz, 3H), 0.99 (dq, J=6.6, 4.5, 3.4 Hz, 2H). ESI MS [M+H]⁺ for C₃₂H₃₂ClF₅N₄O₅ calcd 683.2, found 683.2.

### Example 83: N-[4-[4-chloro-2-(3,3-difluorocyclobutanecarbonyl)phenyl]-6-(2,2-difluoroethoxy)pyridin-2-yl]-1-cyclopropyl-5-[[[(2S)-2-methoxypropyl]amino]methyl]-2-oxopyridine-3-carboxamide

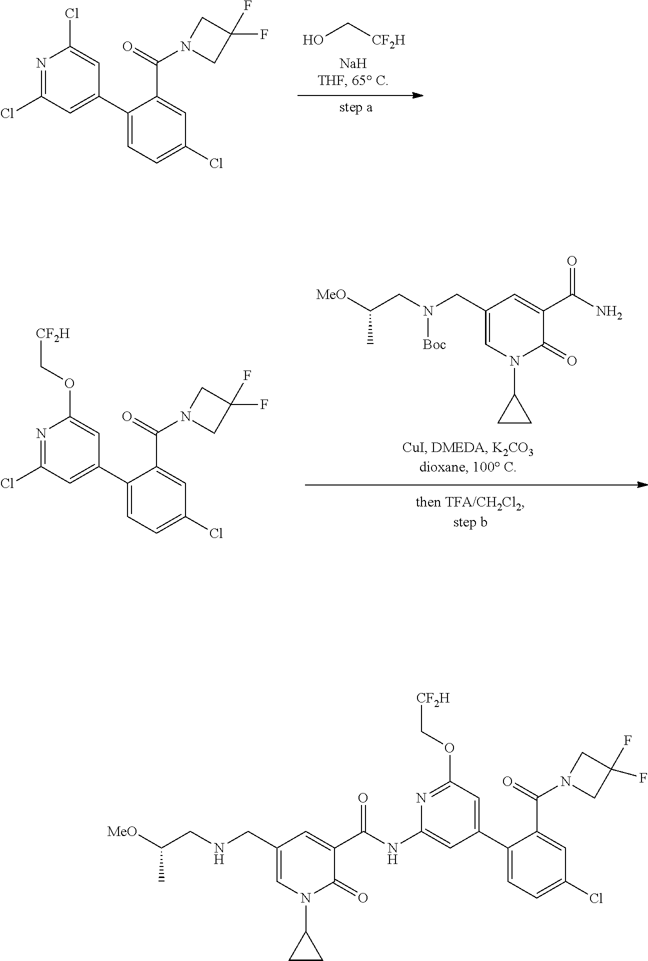 [SMILES: O=C(c1cc(Cl)ccc1-c1cc(Cl)nc(Cl)c1)N1CC(F)(F)C1 ; OCC(F)F ; O=C(c1cc(Cl)ccc1-c1cc(Cl)nc(OCC(F)F)c1)N1CC(F)(F)C1 ; CO[C@@H](C)CN(Cc1cc(C(N)=O)c(=O)n(C2CC2)c1)C(=O)OC(C)(C)C ; CO[C@@H](C)CNCc1cc(C(=O)Nc2cc(-c3ccc(Cl)cc3C(=O)N3CC(F)(F)C3)cc(OCC(F)F)n2)c(=O)n(C2CC2)c1]

Step a: To a solution of 2,2-difluoroethanol (180 mg, 2.22 mmol, 1.20 equiv.) in THF mL, 0.2 M) was added NaH (90 mg, 60% in mineral oil, 2.22 mmol, 1.20 equiv.) at 0° C. The cooling bath was removed, and the mixture was stirred at the room temperature for 10 minutes, until all NaH was dissolved. [5-chloro-2-(2,6-dichloropyridin-4-yl)phenyl]-(3,3-difluoroazetidin-1-yl)methanone (700 mg, 1.85 mmol, 1.0 equiv., prepared according to example 82) was added to the reaction mixture and stirred for 12 h at 65° C. Then, the reaction mixture was quenched with water (4.0 mL), diluted with EtOAc (8.0 mL), washed with sat. aq. NH₄Cl (5.0 ml), and then water (5.0 mL). The organic phase was dried over Na₂SO₄, concentrated and the crude residue was purified by column chromatography (SiO₂, EtOAc in hexanes, 0 to 20%) to give desired product.

Step b: A solution of the product from step a (81 mg, 0.18 mmol, 1.0 equiv.), tert-butyl N-[(5-carbamoyl-1-cyclopropyl-6-oxopyridin-3-yl)methyl]-N-[(2S)-2-methoxypropyl]carbamate (70 mg, 0.18 mmol, 1.0 equiv., prepared according to example 89) and K₂CO₃ (152 mg, 1.10 mmol, 6.0 equiv.) in dioxane (3.70 ml, 0.05 M) was degassed with a stream of bubbling nitrogen for ten minutes. CuI (70 mg, 0.36 mmol, 2.0 equiv.) and DMEDA (82 μL, 0.36 mmol, 2.0 equiv.) were added, and the reaction was heated for 16 h at 100° C. Then the mixture was cooled to room temperature, diluted with aq. NH₄Cl (2 mL) and extracted with EtOAc (2×5 mL). The combined organic phase was washed with water (2×4 mL) and brine (4 mL), dried over Na₂SO₄, and concentrated to dryness. The crude residue was purified by reversed-phase column chromatography (C18, 10-100% CH₃CN in water with 0.1% formic acid) to afford corresponding coupling product. The residual material was then treated with TFA/DCM (v/v 1:10, 2 mL) at room temperature for 1 h. The mixture was concentrated under vacuum, and the crude residue was then purified by reversed-phase column chromatography (C18, 10-100% CH₃CN in water with 0.1% formic acid) to afford the title compound. ¹H NMR (400 MHz, CDCl₃) δ 12.35 (s, 1H), 8.55 (d, J=2.6 Hz, 1H), 8.12 (d, J=1.3 Hz, 1H), 7.65 (d, J=2.7 Hz, 1H), 7.48 (d, J=2.0 Hz, 3H), 6.63 (d, J=1.3 Hz, 1H), 6.11 (tt, J=55.5, 4.2 Hz, 1H), 4.58 (td, J=13.6, 4.2 Hz, 2H), 4.36 (t, J=11.9 Hz, 2H), 4.04 (t, J=11.5 Hz, 2H), 3.64 (d, J=2.0 Hz, 2H), 3.46 (dtt, J=11.7, 7.6, 4.3 Hz, 2H), 3.34 (s, 3H), 2.68-2.55 (m, 2H), 1.86 (s, 2H), 1.34-1.20 (m, 2H), 1.14 (d, J=6.2 Hz, 3H), 0.97 (dq, J=4.7, 2.8, 2.2 Hz, 2H). ESI MS [M+H]⁺ for C₃₂H₃₃ClF₄N₄O₅ calcd 665.2, found 665.2.

### Example 84: N-(6-cyclopropyl-4-{2-[(3,3-difluoro-1-azetidinyl)carbonyl]-4-difluoromethoxyphenyl}-2-pyridyl)-5-[({[(R)-tetrahydro-2-furyl]methyl}amino)methyl]-1-cyclopropyl-2-oxo-1,2-dihydronicotinamide

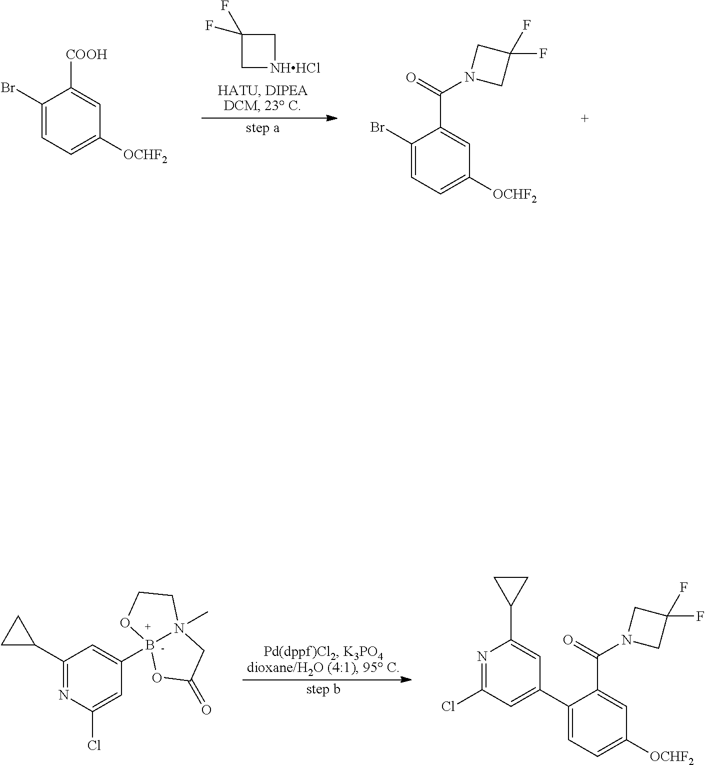

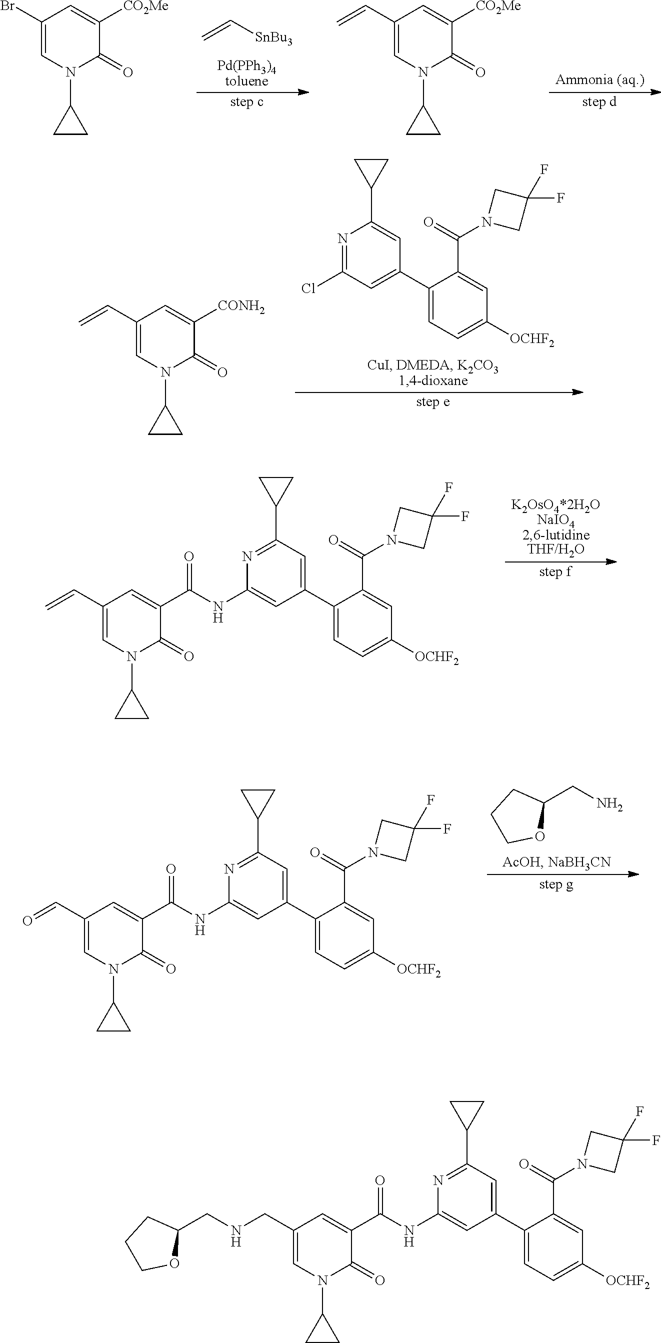

Step a: To a solution of a 2-bromo-5-(difluoromethoxy)benzoic acid (1.02 g, 3.82 mmol, 1 equiv.) in DCM (10 mL, 0.3 M) was added i-Pr₂NEt (1.6 mL, 11.46 mmol, 3 equiv.), HATU (2.9 g, 7.64 mmol, 2 equiv.) and 3,3-difluoroazetidine hydrochloride (0.75 g, 5.73 mmol, 1.5 equiv.). The reaction mixture was stirred overnight at room temperature and diluted with EtOAc and water. The organic phase was separated, washed with brine, dried over Na₂SO₄ and concentrated to dryness under reduced pressure. The crude residue was purified by silica gel flash column chromatography (SiO₂, hextane in EtOAc, 0 to 60%) to afford [2-bromo-5-(difluoromethoxy)phenyl]-(3,3-difluoroazetidin-1-yl)methanone.

Step b: To a solution of the product of step a (0.2 g, 0.5848 mmol, 1.0 equiv.), 1-(2-chloro-6-cyclopropylpyridin-4-yl)-5-methyl-2,8-dioxa-5-azonia-1-boranuidabicyclo[3.3.0]octane-3,7-dione (0.18 g, 0.5848 mmol, 1.0 equiv.) and K₃PO₄ (0.373 g, 1.7544 mmol, 3.0 equiv.) in dioxane (6 mL, 0.1 M) and H₂O (1 mL, 0.1 M) was added Pd(dppf)Cl₂ (45 m g, 0.0585 mmol, 0.1 equiv.) at rt under nitrogen atmosphere. The resulting mixture was stirred for 1 h at 95° C. The mixture was allowed to cool down to rt. The reaction was quenched with sat. aq. NH₄Cl solution, the organic phase was separated, and the aqueous layer was extracted with EtOAc. The combined organic phase was dried over Na₂SO₄, concentrated and the crude residue was purified by column chromatography (SiO₂, hextane in EtOAc, 0 to 60%) to afford methyl 2-(2-chloro-6-cyclopropylpyridin-4-yl)-5-cyanobenzoate.

Step c: A mixture of methyl 5-bromo-1-cyclopropyl-2-oxopyridine-3-carboxylate (8.06 g, 29.6 mmol, 1 equiv.), vinyl tributyltin (11.3 mL, 38.5 mmol, 1.3 equiv.), Pd(PPh₃)₄ (3.42 g, 2.96 mmol, 0.1 equiv.) and toluene (148 mL, 0.05 M) was loaded in a 100 mL round bottom flask equipped with a stirring bar, reflux condenser and nitrogen inlet. The resulting mixture was degassed under vacuum and backfilled with nitrogen (repeated 3 times), then refluxed for 3 h. Once TLC analysis indicated complete conversion of the starting material the heating bath was removed, and the reaction mixture was allowed to cool to room temperature. Then 1M aq. potassium fluoride solution was added (50 mL), and the resulting mixture was vigorously stirred at room temperature overnight. The formed precipitate was removed by filtration through a pad of CELITE®. The filtrate was transferred into separatory funnel, the organic layer was separated, and the aqueous phase was additionally extracted with EtOAc (2×40 mL). Combined organic solution was dried over Na₂SO₄ and concentrated to dryness under reduced pressure. The crude product was purified by column chromatography (SiO₂, 0-100% EtOAc gradient in hexanes) to produce methyl 1-cyclopropyl-5-ethenyl-2-oxopyridine-3-carboxylate.

Step d: A mixture of the product from step c (5 g, 22.7 mmol, 1 equiv.) was treated with NH₃ in MeOH (32 mL, 10 equiv., 7 N) overnight. The mixture was then concentration to afford 1-cyclopropyl-5-ethenyl-2-oxopyridine-3-carboxamide, which is used for the next step without further purification.

Step e: To a mixture of the product of step d (425 mg, 1.9324 mmol, 1.0 equiv.), the product of step b (800 mg, 1.9324 mmol, 1.0 equiv.) in dioxane (5.0 mL) was added CuI (380 mg, 1.9324 mmol, 1.0 equiv.), N,N′-dimethylethylenediamine (0.91 ml, 7.7296 mmol, 4.0 equiv.) and K₂CO₃ (1.5 g, 9.662 mmol, 5.0 equiv.) The resulting mixture was heated at 110° C. overnight. The reaction mixture was cooled to room temperature, diluted with EtOAc, washed with H₂O and brine, filtered through Na₂SO₄, and concentrated. The crude product was purified by column chromatography (SiO₂, 0-100% EtOAc gradient in hexanes) to produce 1-cyclopropyl-N-[6-cyclopropyl-4-[2-(3,3-difluoroazetidine-1-carbonyl)-4-(difluoromethoxy)phenyl]pyridin-2-yl]-5-ethenyl-2-oxopyridine-3-carboxamide.

Step f: To a solution the product of step e (0.3 g, 0.5155 mmol, 1 equiv.) in a mixture of THF (3 mL) and water (3 mL), 2,4-lutidine (111 mg, 1.0309 mmol, 2 equiv.) was added. The resulting mixture was placed in cold water bath (˜15° C.), then sodium periodate (0.442 g, 2.062 mmol, 4 equiv.) and K₂OsO₄*2H₂O (10 mg, 0.026 mmol, 0.05 equiv.) were added sequentially. The resulting mixture was vigorously stirred for 4 h. During the reaction time a significant amount of white precipitate formed. Once TLC analysis confirmed complete reaction, the mixture was diluted with water (30 mL) and EtOAc (40 mL). The aqueous phase was extracted with EtOAc (3×30 mL). Combined organic phase was washed with brine (100 mL), dried over Na₂SO₄ and concentrated. The crude product was purified by column chromatography (SiO₂, 0-100% EtOAc gradient in hexanes) to produce 1-cyclopropyl-N-[6-cyclopropyl-4-[2-(3,3-difluoroazetidine-1-carbonyl)-4-(difluoromethoxy)phenyl]pyridin-2-yl]-5-formyl-2-oxopyridine-3-carboxamide.

Step g: To the solution of step f (50 mg, 0.08576 mmol, 1.0 equiv.) in DCM (3 mL, 0.03 M) was added [(2S)-oxolan-2-yl]methanamine (18 mg, 0.1715 mmol, 2.0 equiv.) and AcOH (1 drop, 1.0 equiv.) and the mixture was stirred for 1 h. NaBH₃CN (12 mg, 0.1715 mmol, 2.0 equiv.) was added and the mixture was stirred at rt for 12 h. The reaction mixture was cooled to room temperature, diluted with DCM, washed with H₂O and brine, filtered through Na₂SO₄, and concentrated. The crude residue was then purified by prep-HPLC (C18, 10-90% CH₃CN in water, 0.1% formic acid) to afford title compound. ¹H NMR (400 MHz, Chloroform-d) δ 12.27 (s, 1H), 8.55 (d, J=2.6 Hz, 1H), 8.24 (d, J=1.5 Hz, 1H), 7.71 (d, J=2.6 Hz, 1H), 7.56 (d, J=9.3 Hz, 1H), 7.28 (d, J=2.9 Hz, 2H), 6.97 (d, J=1.5 Hz, 1H), 6.59 (t, J=73.0 Hz, 1H), 4.34 (t, J=11.9 Hz, 2H), 4.03 (qd, J=7.4, 3.4 Hz, 1H), 3.95 (t, J=11.6 Hz, 2H), 3.85 (dt, J=8.3, 6.6 Hz, 1H), 3.79-3.74 (m, 1H), 3.73 (s, 2H), 3.46 (tt, J=7.5, 4.2 Hz, 1H), 2.78 (dd, J=12.0, 3.4 Hz, 1H), 2.66 (dd, J=12.0, 8.0 Hz, 1H), 2.07-1.94 (m, 2H), 1.90 (p, J=6.7 Hz, 2H), 1.56 (dq, J=11.6, 7.6 Hz, 1H), 1.25 (q, J=6.9 Hz, 2H), 1.09 (dt, J=6.2, 3.1 Hz, 2H), 1.04-0.90 (m, 4H). ESI MS [M+H]⁺ for C₃₄H₃₆F₄N₅O₅, calcd 670.3, found 670.2.

### Example 85: N-(6-cyclopropyl-4-{2-[(3,3-difluoro-1-azetidinyl)carbonyl]-4-difluoromethoxyphenyl}-2-pyridyl)-1-cyclopropyl-5-[(isobutylamino)methyl]-2-oxo-1,2-dihydronicotinamide

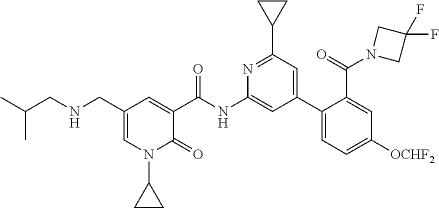 [SMILES: CC(C)CNCc1cc(C(=O)Nc2cc(-c3ccc(OC(F)F)cc3C(=O)N3CC(F)(F)C3)cc(C3CC3)n2)c(=O)n(C2CC2)c1]

The title compound was prepared in a similar fashion to that described for Example 84 using isobutylamine in step g. ¹H NMR (400 MHz, Chloroform-d) δ 12.24 (s, 1H), 8.56 (d, J=2.6 Hz, 1H), 8.20 (d, J=1.5 Hz, 1H), 7.77 (d, J=2.6 Hz, 1H), 7.56 (d, J=9.3 Hz, 1H), 7.33-7.27 (m, 2H), 6.97 (d, J=1.5 Hz, 1H), 6.60 (t, J=73.0 Hz, 1H), 4.34 (t, J=11.9 Hz, 2H), 3.95 (t, J=11.5 Hz, 2H), 3.77 (s, 2H), 3.44 (dt, J=7.3, 3.4 Hz, 1H), 2.55 (d, J=6.9 Hz, 2H), 1.99 (tq, J=11.6, 6.8, 5.7 Hz, 1H), 1.84 (dt, J=13.4, 6.7 Hz, 1H), 1.23 (q, J=6.9 Hz, 2H), 1.09 (dt, J=6.2, 3.1 Hz, 2H), 0.95 (dd, J=16.3, 4.8 Hz, 10H). ESI MS [M+H]⁺ for C₃₃H₃₆F₄N₅O₄, calcd 642.3, found 642.2.

### Example 86: N-(6-cyclopropyl-4-{2-[(3,3-difluoro-1-azetidinyl)carbonyl]-4-difluoromethoxyphenyl}-2-pyridyl)-5-{[(S)-1-cyclobutylethylamino]methyl}-1-cyclopropyl-2-oxo-1,2-dihydronicotinamide

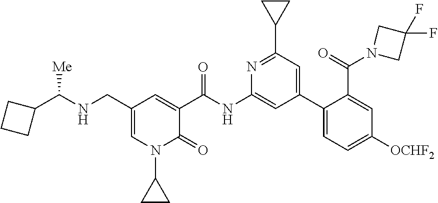 [SMILES: C[C@H](NCc1cc(C(=O)Nc2cc(-c3ccc(OC(F)F)cc3C(=O)N3CC(F)(F)C3)cc(C3CC3)n2)c(=O)n(C2CC2)c1)C1CCC1]

The title compound was prepared in a similar fashion to that described for Example 84 using (S)-1-cyclobutyl-ethylamine in step g. ¹H NMR (400 MHz, Chloroform-d) δ 12.25 (s, 1H), 8.55 (d, J=2.7 Hz, 1H), 8.22 (d, J=1.5 Hz, 1H), 7.79 (d, J=2.6 Hz, 1H), 7.64-7.45 (m, 1H), 7.28 (s, 2H), 6.98 (d, J=1.5 Hz, 1H), 6.59 (t, J=73.0 Hz, 1H), 4.34 (t, J=12.0 Hz, 2H), 3.95 (t, J=11.5 Hz, 2H), 3.79 (d, J=13.4 Hz, 1H), 3.67 (d, J=13.4 Hz, 1H), 3.46 (tt, J=7.6, 4.1 Hz, 1H), 2.82-2.64 (m, 1H), 2.35 (dt, J=16.3, 8.1 Hz, 1H), 2.09-1.93 (m, 2H), 1.88 (h, J=8.4, 7.6 Hz, 1H), 1.80-1.60 (m, 4H), 1.23 (m, 2H), 1.13-1.02 (m, 5H), 0.96 (td, J=7.7, 7.0, 3.2 Hz, 4H). ESI MS [M+H]⁺ for C₃₅H₃₈F₄N₅O₄, calcd 668.3, found 668.2.

### Example 87: N-[6-cyclopropyl-4-[2-(3,3-difluoroazetidine-1-carbonyl)-4-(difluoromethoxy)phenyl]pyridin-2-yl]-5-[[[(2S)-2-methoxypropyl]amino]methyl]-1-methyl-2-oxopyridine-3-carboxamide

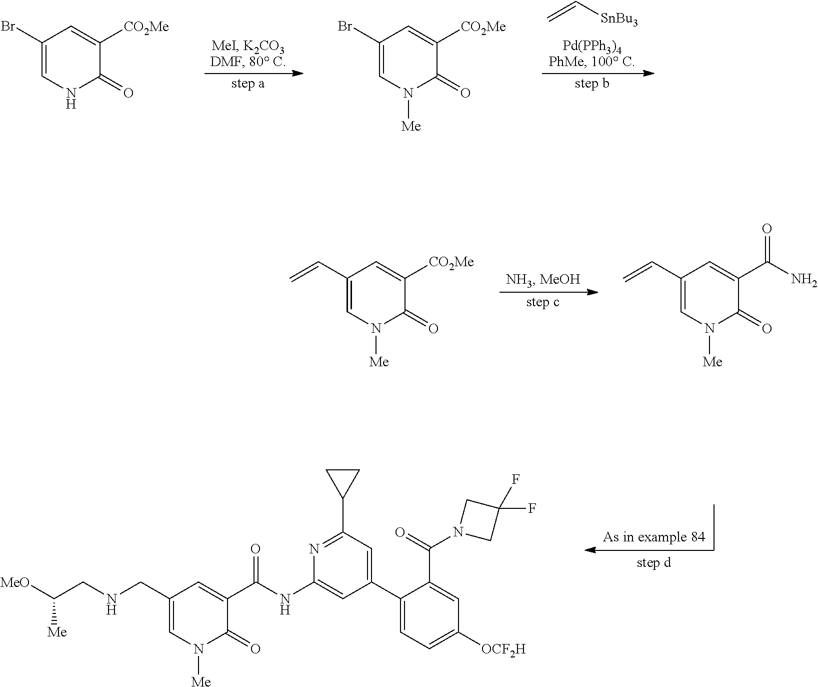

Step a: To a suspension of methyl 5-bromo-2-oxo-1H-pyridine-3-carboxylate (5.0 g, 21.6 mmol, 1.0 equiv.) and K₂CO₃ (8.9 g, 64.7 mmol, 3.0 equiv.) in DMF (100 mL) was added MeI (2.0 mL, 32.3 mmol, 1.5 equiv.). The reaction mixture was heated to 80° C. and stirred for 1 hour. The reaction was cooled to room temperature and partitioned between EtOAc (250 mL) and water (500 mL). The aqueous layer was extracted with EtOAc (2×100 mL) and the combined organics were dried over Na₂SO₄, filtered, and concentrated under vacuum. The crude residue was purified by column chromatography (SiO₂, 50-100% EtOAc in hexanes) to afford the desired product.

Step b: To a suspension of the product from step a (2.7 g, 11.0 mmol, 1.0 equiv.) in PhMe (100 mL) was added tributyl(ethenyl)stannane (4.8 mL, 16.5 mmol, 1.5 equiv.). The resulting mixture was sparged with N₂ for 10 minutes. Pd(PPh₃)₄ (630 mg, 1.1 mmol, 0.05 equiv.) was added and the reaction was heated to 100° C. for 16 hours at which point it was cooled to room temperature and directly concentrated under vacuum. The crude residue was purified by column chromatography (SiO₂, 0-100% EtOAc in hexanes) to afford the desired product.

Step c: The product from step b (729 mg, 3.8 mmol, 1.0 equiv.) was dissolved in 7M NH₃ in MeOH (15 mL) and the resulting solution was stirred at room temperature for 4 hours. The reaction mixture was directly concentrated under vacuum and the crude residue was used without further purification.

Step d: The title compound was prepared in a similar fashion to that described for Example 84 using (2S)-2-methoxypropan-1-amine in step g. ¹H NMR (400 MHz, Chloroform-d) δ 12.32 (s, 1H), 8.56 (d, J=2.6 Hz, 1H), 8.25 (d, J=1.4 Hz, 1H), 7.64 (d, J=2.6 Hz, 1H), 7.59-7.52 (m, 1H), 7.31-7.23 (m, 2H), 6.97 (d, J=1.5 Hz, 1H), 6.59 (t, J=73.1 Hz, 1H), 4.34 (t, J=11.9 Hz, 2H), 3.96 (t, J=11.6 Hz, 2H), 3.70 (s, 3H), 3.66 (d, J=4.3 Hz, 2H), 3.53-3.44 (m, 1H), 3.35 (s, 3H), 2.68-2.57 (m, 2H), 2.07-1.96 (m, 1H), 1.15 (d, J=6.2 Hz, 3H), 1.13-1.06 (m, 2H), 0.98-0.91 (m, 2H). ESI MS [M+H]⁺ for C₃₁H₃₃F₄N₅O₅ calcd 632.3 found 632.3.

### Example 88: N-[6-cyclopropyl-4-[2-(3,3-difluoroazetidine-1-carbonyl)-4-(difluoromethoxy)phenyl]pyridin-2-yl]-1-methyl-2-oxo-5-[[[(2S)-oxolan-2-yl]methylamino]methyl]pyridine-3-carboxamide

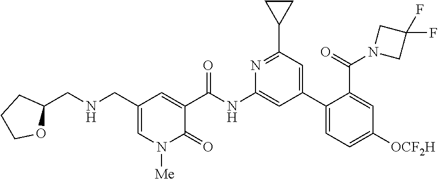

The title compound was prepared in a similar fashion to that described for Example 84 using 5-ethenyl-1-methyl-2-oxopyridine-3-carboxamide in step e. ¹H NMR (400 MHz, Chloroform-d) δ 12.32 (s, 1H), 8.56 (d, J=2.6 Hz, 1H), 8.24 (d, J=1.5 Hz, 1H), 7.67 (d, J=2.6 Hz, 1H), 7.59-7.51 (m, 1H), 7.32-7.24 (m, 2H), 6.97 (d, J=1.5 Hz, 1H), 6.59 (t, J=73.1 Hz, 1H), 4.34 (t, J=11.9 Hz, 2H), 4.06-3.90 (m, 3H), 3.85 (dt, J=8.3, 6.6 Hz, 1H), 3.75 (dt, J=8.3, 6.7 Hz, 1H), 3.70 (s, 5H), 2.74 (dd, J=12.0, 3.5 Hz, 1H), 2.64 (dd, J=12.0, 7.8 Hz, 1H), 2.10-1.85 (m, 4H), 1.12-1.06 (m, 2H), 0.98-0.90 (m, 2H). ESI MS [M+H]⁺ for C₃₂H₃₃F₄N₅O₅ calcd 644.3 found 644.3.

### Example 89: N-(6-cyclopropyl-4-{2-[(3,3-difluoro-1-azetidinyl)carbonyl]-4-difluoromethoxyphenyl}-2-pyridyl)-5-{[(S)-2-methoxypropylamino]methyl}-1-cyclopropyl-2-oxo-1,2-dihydronicotinamide

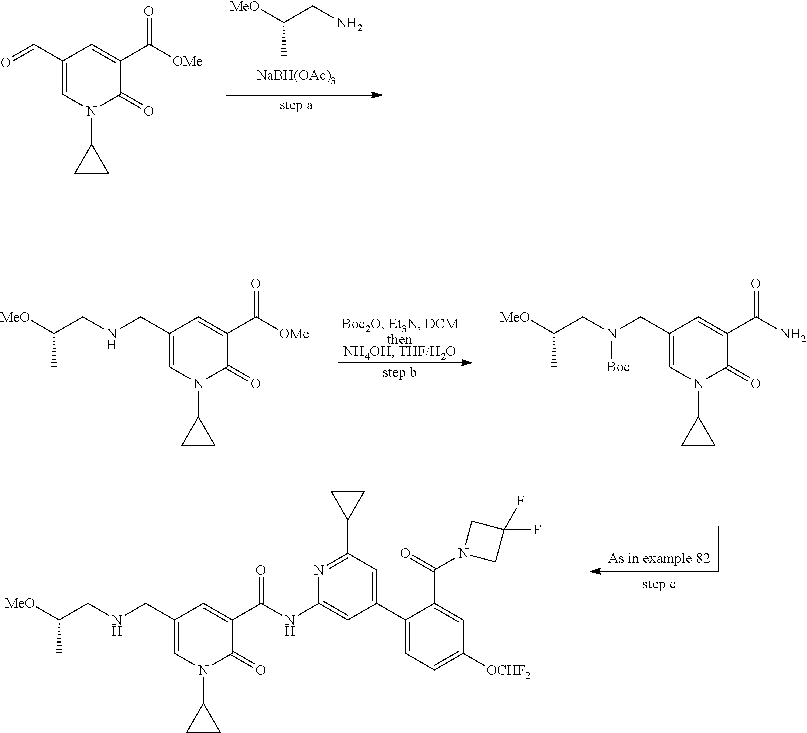

Step a: To the solution of methyl 1-cyclopropyl-5-formyl-2-oxopyridine-3-carboxylate (7.5 g, 33.7 mmol, 1.0 equiv., prepared according to example 1) in dichloromethane (100 mL, 0.3 M) was added (2S)-2-methoxypropan-1-amine (3 g, 33.7 mmol, 1.0 equiv.) and AcOH (1.93 mL, 33.7 mmol, 1.0 equiv.) and the mixture was stirred at 23° C. for 10 mins. NaBH(OAc)₃ (11 g, 50.6 mmol, 1.5 equiv.) was added and the mixture was stirred at 23° C. overnight. The reaction was quenched with aq. sat. NaHCO₃ (3 mL) and diluted with EtOAc (10 mL). The organic phase was separated, and the aqueous layer was additionally extracted with EtOAc (2×10 mL). The combined organic extract was dried over Na₂SO₄ and concentrated to dryness under reduced pressure. The crude residue was purified by column chromatography (SiO₂, 0-25% MeOH in dichlorormethane) to afford the desired amination product methyl 1-cyclopropyl-5-[[[(2S)-2-methoxypropyl]amino]methyl]-2-oxopyridine-3-carboxylate.

Step b: To a solution of the product from step a (6.5 g, 22.1 mmol, 1.0 equiv.) in DCM (200 mL, 0.1 M) was added triethylamine (6.2 ml, 44.2 mmol, 2.0 equiv.) and Boc₂O (7.5 g, 33.2 mmol, 1.5 equiv.) and the mixture was stirred at 23° C. overnight. Then the mixture was concentrated to dryness under vacuum. The crude residue was dissolved in THF and water (90 mL, 2:1 v/v). Aq. conc. NH₄OH (80 ml) was added dropwise over 5 min, and the mixture was stirred overnight at 23° C. The product was extracted with EtOAc (2×50 mL), the combined organic phase was washed with water, dried over Na₂SO₄ and concentrated to dryness under reduced pressure. The crude residue was purified by column chromatography (SiO₂, 0-80% MeOH gradient in DCM) to yield tert-butyl N-[(5-carbamoyl-1-cyclopropyl-6-oxopyridin-3-yl)methyl]-N-[(2S)-2-methoxypropyl]carbamate.

Step c: The title compound was prepared in a similar fashion to that described for step d of Example 82 using the product of Example 84, step b. ¹H NMR (400 MHz, Chloroform-d) δ 12.29 (s, 1H), 8.55 (d, J=2.6 Hz, 1H), 8.25 (d, J=1.4 Hz, 1H), 7.75-7.51 (m, 2H), 7.28 (d, J=3.0 Hz, 2H), 6.97 (d, J=1.4 Hz, 1H), 6.59 (t, J=73.0 Hz, 1H), 4.34 (t, J=11.9 Hz, 2H), 3.95 (t, J=11.6 Hz, 2H), 3.65 (d, J=2.4 Hz, 2H), 3.47 (ddt, J=11.2, 7.7, 4.5 Hz, 2H), 3.35 (s, 3H), 2.74-2.51 (m, 2H), 2.00 (tt, J=8.4, 4.7 Hz, 1H), 1.26 (t, J=3.7 Hz, 2H), 1.15 (d, J=6.2 Hz, 3H), 1.09 (dt, J=6.4, 3.3 Hz, 2H), 0.96 (ddp, J=11.3, 7.0, 4.1 Hz, 4H). ESI MS [M+H]⁺ for C₃₃H₃₆F₄N₅O₅, calcd 658.3, found 658.2.

### Example 90: N-(4-{4-chloro-2-[(3,3-difluoro-1-azetidinyl)carbonyl]phenyl}-6-cyclopropyl-2-pyridyl)-5-[({[(S)-tetrahydro-2-furyl]methyl}amino)methyl]-1-cyclopropyl-2-oxo-1,2-dihydronicotinamide

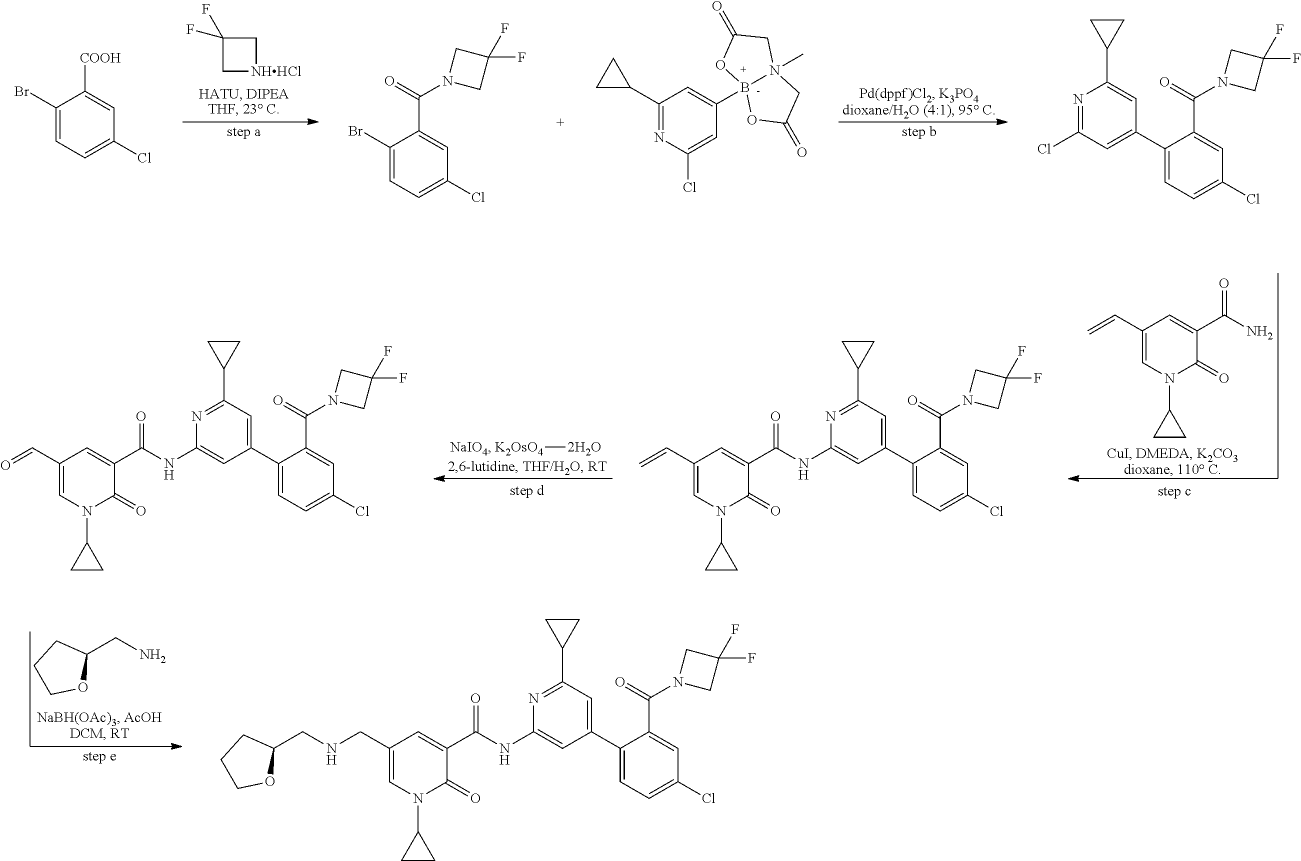

Step a: To a solution of 2-bromo-5-chlororobenzoic acid (1.0 g, 4.2 mmol, 1 equiv.) in THF (21 mL, 0.2 M) was added DIPEA (1.4 mL, 8.4 mmol, 2 equiv.), HATU (2.4 g, 6.3 mmol, 1.5 equiv.) and 3,3-difluoroazetidine hydrochloride (0.81 g, 20.54 mmol, 1.5 equiv.). The reaction mixture was stirred overnight at room temperature and diluted with EtOAc (100 mL) and water (50 mL). The organic phase was separated, washed with brine (50 mL), dried over Na₂SO₄ and concentrated to dryness under reduced pressure. The crude residue was purified by silica gel flash column chromatography (SiO₂, 0-80% EtOAc gradient in hexane) to afford the desired product.

Step b: A 40 mL vial was charged with the product from step a (0.61 g, 2.0 mmol. 1 equiv.), 1-(2-chloro-6-cyclopropylpyridin-4-yl)-5-methyl-2,8-dioxa-5-azonia-1-boranuidabicyclo[3.3.0]octane-3,7-dione (0.62 g, 2.0 mmol, 1 equiv.), and K₃PO₄ (1.27 g, 6.0 mmol, 3 equiv.). The reagents were suspended in a mixture of dioxane/water (20 mL, 4:1 v/v, 0.1 M), and the resulting solution was sparged with N₂ for 10 minutes. Then Pd(dppf)Cl₂ (146 mg, 0.2 mmol, 10%) was added, and the mixture was heated to 95° C. for 4 hours. The reaction mixture was partitioned between EtOAc (50 mL) and water (50 mL). The organic phase was separated, and the aqueous phase was additionally extracted with EtOAc (3×50 mL). The combined organics were dried over Na₂SO₄ and concentrated to dryness under reduced pressure. The crude residue was purified via column chromatography (SiO₂, 0-80% EtOAc gradient in hexane) to afford [5-chloro-2-(2-chloro-6-cyclopropylpyridin-4-yl)phenyl]-(3,3-difluoroazetidin-1-yl)methanone as the desired product.

Step c: To a suspension of the product from step b (0.31 g, 0.82 mmol, 1.0 equiv.), 1-cyclopropyl-5-ethenyl-2-oxopyridine-3-carboxamide (0.16 g, 0.82 mmol, 1.0 equiv.), and K₂CO₃ (0.34 g, 2.46 mmol, 3.0 equiv.) in dioxane (16 mL) was added CuI (0.15 g, 0.82 mmol, 1.0 equiv.) and DMEDA (0.17 mL, 1.64 mmol, 2.0 equiv.). The reaction mixture was heated to 110° C. and stirred for 16 hours, at which point it was quenched with a 1:1 mixture of water/brine (50 mL) and extracted with EtOAc (2×75 mL). The combined organics were dried over Na₂SO₄, filtered, and concentrated under a vacuum. The crude residue was purified by column chromatography (SiO₂, EtOAc in hexanes, 0 to 100%) to afford the desired product.

Step d: To a solution of the product from step c (0.24 g, 0.43 mmol, 1.0 equiv.) in a 1:1 mixture of THF/H₂O (3 mL) was added 2,6-lutidene (0.1 mL, 0.86 mmol, 2.0 equiv.) followed by NaIO₄ (0.37 g, 1.74 mmol, 4.0 equiv.) and K₂OsO₄*2H₂O (15.8 mg, 0.04 mmol, 0.1 equiv.). The reaction mixture was stirred for 4 hours at room temperature, at which point it was quenched with saturated aqueous Na₂S₂O₃ (20 mL) and extracted with EtOAc (2×50 mL). The combined organics were dried over Na₂SO₄, filtered, and concentrated under a vacuum. The crude residue was used directly without further purification.

Step d: To a solution of the product from step e (27.6 mg, 0.05 mmol, 1.0 equiv.) in DCM (1.0 mL) was added [(2S)-oxolan-2-yl]methanamine (10 mg, 0.1 mmol, 2.0 equiv.) followed by sodium triacetoxyborohydride (30 mg, 0.13 mmol, 2.5 equiv.) and AcOH (8 uL, 0.13 mmol, 2.5 equiv.). The reaction mixture was stirred for 16 hours at room temperature, at which point it was quenched with saturated aqueous NaHCO₃ (10 mL) and extracted with EtOAc (2×25 mL). The combined organics were dried over Na₂SO₄, filtered, and concentrated under a vacuum. The crude residue was purified by reverse phase prep-HPLC (C18 column, 5 to 60% MeCN/H₂O) to afford the desired product. ¹H NMR (400 MHz, Chloroform-d) δ 12.28 (s, 1H), 8.55 (d, J=2.6 Hz, 1H), 8.24 (d, J=1.5 Hz, 1H), 7.68 (d, J=2.6 Hz, 1H), 7.52-7.47 (m, 3H), 6.97 (d, J=1.5 Hz, 1H), 4.33 (t, J=11.9 Hz, 2H), 4.06-3.91 (m, 3H), 3.89-3.81 (m, 1H), 3.79-3.67 (m, 3H), 3.49-3.43 (m, 1H), 2.78-2.72 (m, 1H), 2.68-2.60 (m, 1H), 2.03-1.84 (m, 5H), 1.62-1.50 (m, 1H), 1.30-1.22 (m, 2H), 1.13-1.05 (m, 2H), 1.00-0.91 (m, 4H). ESI MS [M+H]⁺ for C₃₃H₃₄ClF₂N₅O₄, calcd 638.2, found 638.2.

### Example 91: N-(4-{4-chloro-2-[(3,3-difluoro-1-azetidinyl)carbonyl]phenyl}-6-ethoxy-2-pyridyl)-5-[({[(S)-tetrahydro-2-furyl]methyl}amino)methyl]-1-cyclopropyl-2-oxo-1,2-dihydronicotinamide

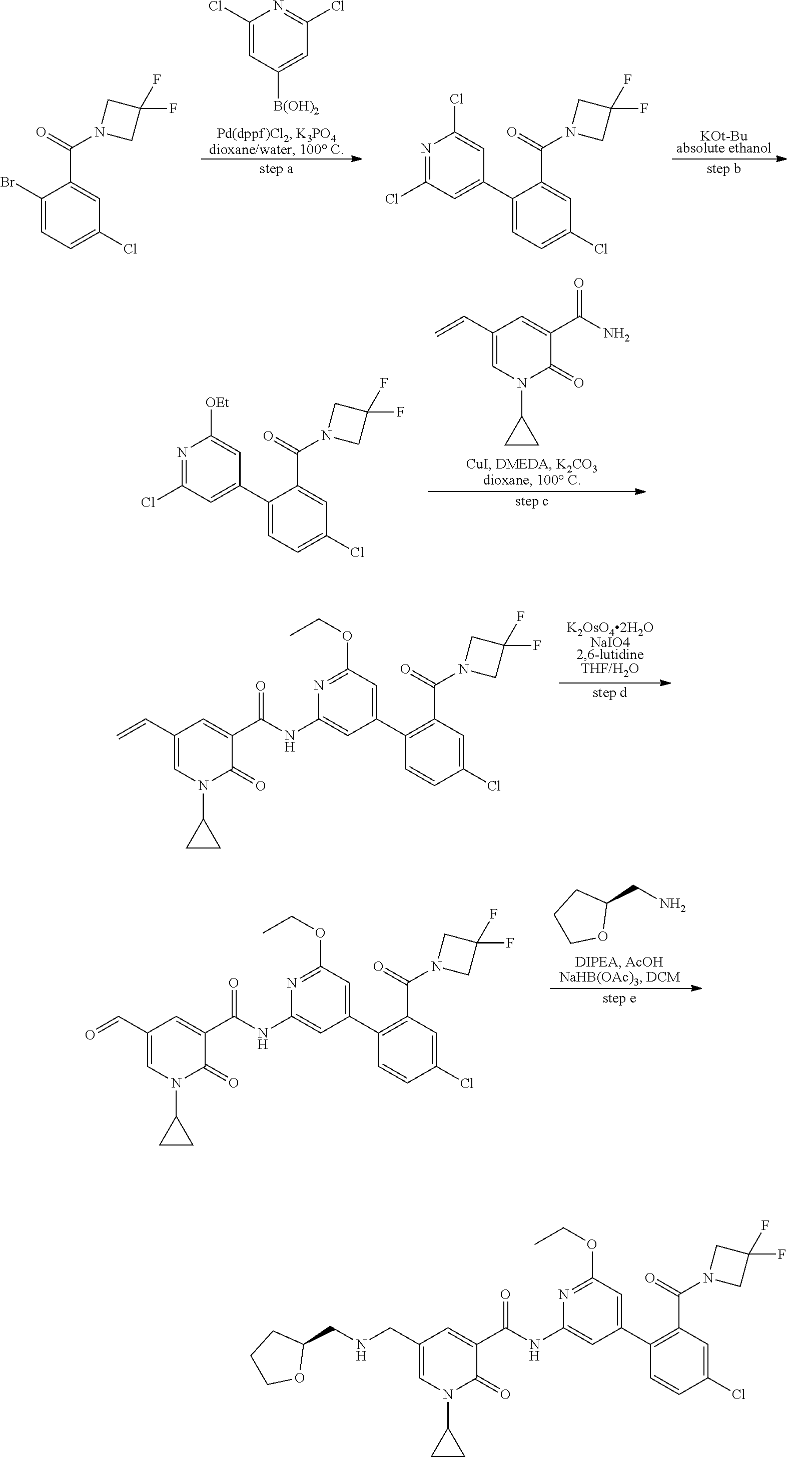

Step a: A 250 mL round bottom flask was charged with (2-bromo-5-chlorophenyl)-(3,3-difluoroazetidin-1-yl)methanone (3.2 g, 10.1 mmol, 1.0 equiv.), (2,6-dichloropyridin-4-yl)boronic acid (5.1 g, 26.6 mmol. 2.6 equiv.) and potassium phosphate (7.0 g, 32.97 mmol, 3.2 equiv.). The reagents were suspended in the 4:1 mixture of dioxane/water (81 mL). Then Pd(dppf)Cl₂ (1.2 g, 1.64 mmol, 0.16 equiv.) was added, and the mixture was heated to 100° C. for 6 hours. The reaction mixture was partitioned between EtOAc and water. The organic phase was separated, and the aqueous phase was additionally extracted with EtOAc. The combined organics were dried over Na₂SO₄ and concentrated to dryness. The crude residue was purified via silica gel flash column chromatography (EtOAc in hexanes, 0 to 100%) to afford [5-chloro-2-(2,6-dichloropyridin-4-yl)phenyl]-(3,3-difluoroazetidin-1-yl)methanone.

Step b: A solution of KOtBu (0.89 g, 7.94 mmol, 3.0 equiv.) in absolute ethanol (13 mL) was stirred for 5 min at room temperature. Then the product from step a (1.0 g, 2.65 mmol, 1.0 equiv.) was added and heated at 90° C. for 30 minutes at which point it was quenched with a ˜1:1 mixture of saturated aqueous NaCl/water and extracted with EtOAc. The combined organics were dried over Na₂SO₄, filtered, and concentrated under vacuum. The crude residue was purified via silica gel flash column chromatography (EtOAc in hexanes, 0 to 100%) to afford the desired product.

Step c: To a mixture of the product from step b (947 mg, 2.44 mmol, 1.0 equiv.) and 1-cyclopropyl-5-ethenyl-2-oxopyridine-3-carboxamide (500 mg, 2.44 mmol, 1.0 equiv.) in dioxane (49 mL, 0.05 M) was added CuI (511 mg, 2.68 mmol, 1.0 equiv.), DMEDA (430 mg, 88.2 mmol, 1.1 equiv.) and K₂CO₃ (2.0 g, 14.64 mmol, 6.0 equiv.). The resulting mixture was heated at 100° C. under N₂ for 40 h under vigorous stirring. The reaction was allowed to cool to rt, diluted with water and EtOAc, the organic phase was separated, and the aqueous layer was additionally extracted with EtOAc. The combined organic phase was dried over MgSO₄, concentrated and the crude residue was purified by column chromatography (EtOAc in hexanes, 0 to 100%) to provide corresponding coupling product.

Step d: The reaction was performed in a similar fashion to Example 84 step f using the product from step c.

Step e: The reaction was performed in a similar fashion to Example 84 step g using the product from step d and (S)-(+)-tetrahydrofurfurylamine to afford the title compound. ¹H NMR (400 MHz, CDCl₃) δ 12.20 (s, 1H), 8.58 (d, J=2.6 Hz, 1H), 8.00 (s, 1H), 7.84 (d, J=2.5 Hz, 1H), 7.53-7.44 (m, 3H), 6.56 (d, J=1.3 Hz, 1H), 4.34 (d, J=12.1 Hz, 2H), 4.10 (d, J=7.5 Hz, 1H), 4.00 (t, J=11.6 Hz, 2H), 3.95-3.80 (m, 4H), 3.74 (q, J=7.2 Hz, 1H), 3.50-3.39 (m, 1H), 2.94 (d, J=11.1 Hz, 1H), 2.82-2.72 (m, 1H), 2.06-1.92 (m, 6H), 1.61-1.50 (m, 2H), 1.23 (q, J=6.9 Hz, 3H), 0.98 (d, J=4.6 Hz, 2H). ESI MS [M+H]⁺ C₃₂H₃₅ClF₂N₅O₅, calcd 642.2, found 642.2

### Example 92: N-(4-{4-chloro-2-[(3,3-difluoro-1-azetidinyl)carbonyl]phenyl}-6-(isopropylamino)-2-pyridyl)-5-[({[(S)-tetrahydro-2-furyl]methyl}amino)methyl]-1-cyclopropyl-2-oxo-1,2-dihydronicotinamide

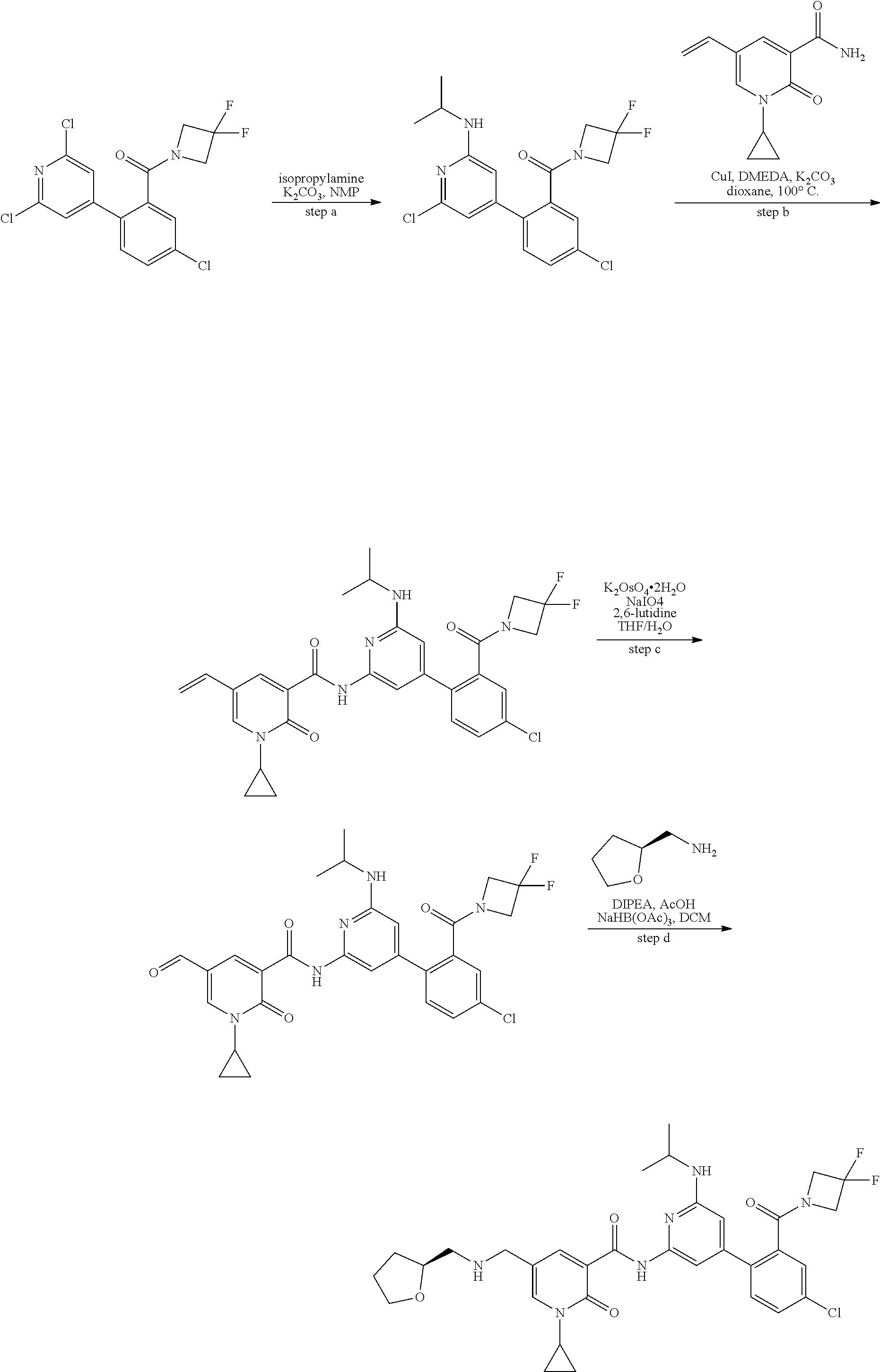

Step a: To a solution of [5-chloro-2-(2,6-dichloropyridin-4-yl)phenyl]-(3,3-difluoroazetidin-1-yl)methanone (1.3 g, 3.48 mmol, 1.0 equiv.) in NMP (18 mL, 0.2 M) was added potassium carbonate (720 mg, 5.22 mmol, 1.5 equiv.) and isopropylamine (1.5 mL, 17.4 mmol, 5.0 equiv). The reaction was stirred at 100° C. for 16 hours in a sealed vial. The reaction mixture was diluted with water and extracted with EtOAc. The combined organics were dried over Na₂SO₄, filtered, and concentrated under vacuum. The crude residue was purified via silica gel flash column chromatography (EtOAc in hexanes, 0 to 20%) to afford the desired product.

Step b: To a mixture of the product from step a (309 mg, 0.73 mmol, 1.0 equiv.) and 1-cyclopropyl-5-ethenyl-2-oxopyridine-3-carboxamide (150 mg, 0.73 mmol, 1.0 equiv.) in dioxane (14.6 mL, 0.05 M) was added CuI (139 mg, 0.73 mmol, 1.0 equiv.), DMEDA (129 mg, 0.73 mmol, 2.0 equiv.) and K₂CO₃ (606.7 g, 4.39 mmol, 6.0 equiv.). The resulting mixture was heated at 100° C. under N₂ for 40 h under vigorous stirring. The reaction was allowed to cool to rt, diluted with water and EtOAc, the organic phase was separated, and the aqueous layer was additionally extracted with EtOAc. The combined organic phase was dried over MgSO₄, concentrated and the crude residue was purified by column chromatography (EtOAc in hexanes, 0 to 100%) to provide corresponding coupling product.

Step d: The reaction was performed in a similar fashion to Example 84 step f using the product from step c.

Step e: The reaction was performed in a similar fashion to Example 84 step g using the product from step d and (S)-(+)-tetrahydrofurfurylamine to afford the title compound. ¹H NMR (400 MHz, CDCl₃) δ 12.24 (s, 1H), 8.53 (d, J=2.6 Hz, 1H), 7.73 (d, J=1.3 Hz, 1H), 7.64 (d, J=2.6 Hz, 1H), 7.54-7.43 (m, 3H), 6.16 (d, J=1.3 Hz, 1H), 4.41 (d, J=8.2 Hz, 1H), 4.30 (t, J=11.9 Hz, 2H), 4.03 (d, J=3.6 Hz, 1H), 4.02-3.89 (m, 3H), 3.85 (dt, J=8.3, 6.6 Hz, 1H), 3.75 (dt, J=8.2, 6.7 Hz, 1H), 3.67 (s, 2H), 3.44 (tt, J=7.6, 4.3 Hz, 1H), 2.73 (dd, J=12.0, 3.5 Hz, 1H), 2.63 (dd, J=12.0, 7.7 Hz, 1H), 2.03-1.85 (m, 3H), 1.63-1.50 (m, 1H), 1.21 (d, J=6.3 Hz, 6H), 1.00-0.91 (m, 2H). ESI MS [M+H]⁺ C₃₂H₃₅ClF₂N₅O₄, calcd 626.3, found 626.3.

### Example 93: N-[4-[4-cyano-2-(3,3-difluoroazetidine-1-carbonyl)phenyl]-6-cyclopropylpyridin-2-yl]-2-oxo-5-[[[(2S)-oxolan-2-yl]methylamino]methyl]-1-(2,2,2-trifluoroethyl)pyridine-3-carboxamide

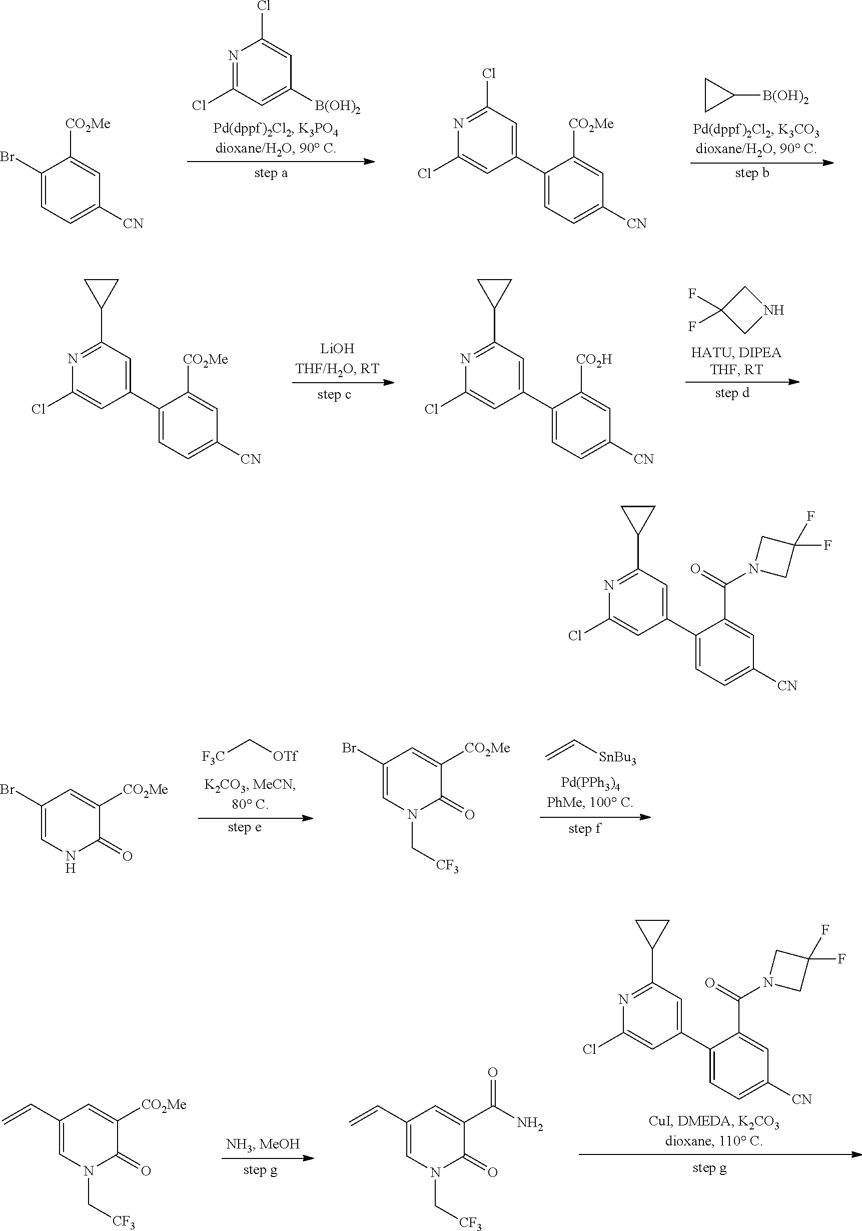

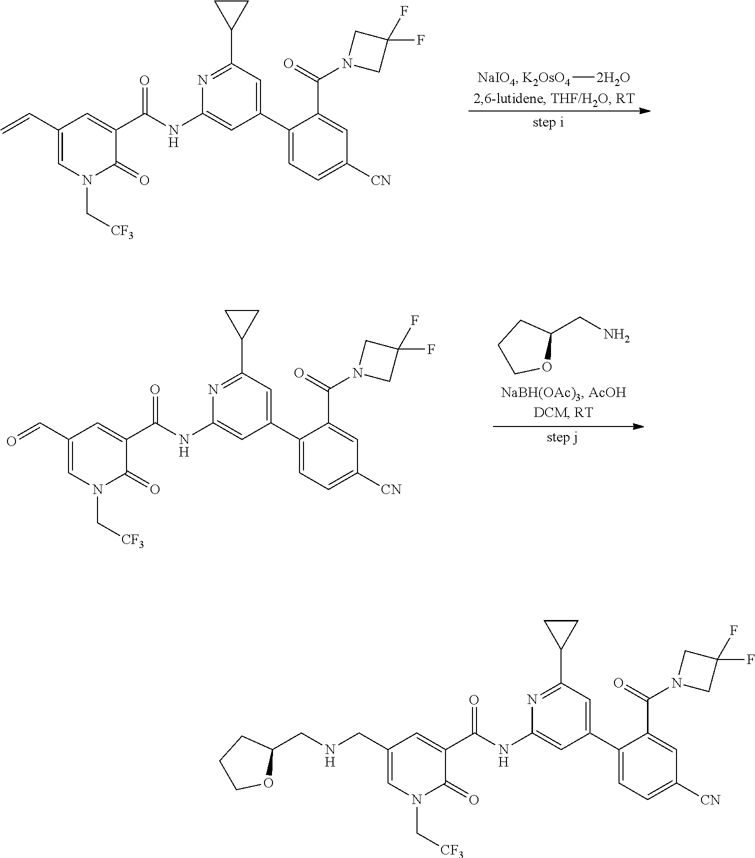

Step a: A suspension of methyl 2-bromo-5-cyanobenzoate (10.0 g, 41.6 mmol, 1.0 equiv.), 2,6-dichloropyridine-4-boronic acid (8.4 g, 43.73 mmol, 1.05 equiv.) and K₃PO₄ (8.83 g, 41.6 mmol, 1.0 equiv.) in a 3:1 mixture of dioxane/H₂O (70 mL) was sparged with N₂ for 10 minutes. Pd(dppf)₂Cl₂ (1.55 g, 2.08 mmol, 0.05 equiv.) was added and the reaction was heated to 90° C. for 2 hours. The reaction was cooled to room temperature and partitioned between EtOAc (300 mL) and water (500 mL). The aqueous layer was extracted with EtOAc (2×100 mL) and the combined organics were dried over Na₂SO₄, filtered, and concentrated under vacuum. The crude residue was purified by column chromatography (SiO₂, 0-40% EtOAc in hexanes) to afford the desired product.

Step b: A suspension of the product from step a (6.0 g, 19.5 mmol, 1.0 equiv.), cyclopropylboronic acid (3.4 g, 39.1 mmol, 2.0 equiv.) and K₂CO₃ (8.2 g, 58.5 mmol, 3.0 equiv.) in a 5:1 mixture of dioxane/H₂O (120 mL) was sparged with N₂ for 10 minutes. Pd(dppf)₂Cl₂ (1.4 g, 1.95 mmol, 0.10 equiv.) was added and the reaction was heated to 90° C. for 12 hours. The reaction was cooled to room temperature and partitioned between EtOAc (300 mL) and water (500 mL). The aqueous layer was extracted with EtOAc (2×100 mL) and the combined organics were dried over Na₂SO₄, filtered, and concentrated under vacuum. The crude residue was purified by column chromatography (SiO₂, 0-40% EtOAc in hexanes) to afford the desired product.

Step c: To a solution of the product from step b (1.0 g, 3.19 mmol, 1.0 equiv.) in a 3:1 mixture of THF/H₂O (20 mL) was added LiOH (230 mg, 9.58 mmol, 3.0 equiv.). The reaction mixture was stirred at room temperature for 3 hours at which point it was quenched with 1 M HCl (pH˜3). The reaction mixture was partitioned between EtOAc (100 mL) and water (200 mL). The aqueous layer was extracted with EtOAc (2×100 mL) and the combined organics were dried over Na₂SO₄, filtered, and concentrated under vacuum. The crude residue was purified by column chromatography (SiO₂, 0-5% MeOH in DCM) to afford the desired product.

Step d: To a solution of the product from step c (10.0 g, 33.6 mmol, 1.0 equiv.) and 3,3-difluoroazetidine hydrochloride (5.6 g, 43.6 mmol, 1.3 equiv.) in THF (100 mL) was added DIPEA (17.5 mL, 101 mmol, 3.0 equiv.) followed by HATU (19.1 g, 50.3 mmol, 1.5 equiv.). The reaction mixture was stirred for 16 hours at room temperature at which point it was partitioned between EtOAc (300 mL) and water (500 mL). The aqueous layer was extracted with EtOAc (2×100 mL) and the combined organics were dried over Na₂SO₄, filtered, and concentrated under vacuum. The crude residue was purified by column chromatography (SiO₂, 0-100% EtOAc in hexanes) to afford the desired product.

Step e: To a suspension of methyl 5-bromo-2-oxo-1H-pyridine-3-carboxylate (10.0 g, 43.1 mmol, 1.0 equiv.) and K₂CO₃ (11.9 g, 86.2 mmol, 2.0 equiv.) in MeCN was added 2,2,2-trifluoroethyl trifluoromethanesulfonate (15.0 g, 64.7 mmol, 1.5 equiv.). The reaction mixture was heated to 80° C. and stirred for 16 hours. The reaction was cooled to room temperature and partitioned between EtOAc (300 mL) and water (500 mL). The aqueous layer was extracted with EtOAc (2×100 mL) and the combined organics were dried over Na₂SO₄, filtered, and concentrated under vacuum. The crude residue was purified by column chromatography (SiO₂, 10-100% EtOAc in hexanes) to afford the desired product.

Step f: To a suspension of the product from step e (10.9 g, 34.7 mmol, 1.0 equiv.) in PhMe (200 mL) was added tributyl(ethenyl)stannane (12 mL, 41.6 mmol, 1.2 equiv.). The resulting mixture was sparged with N₂ for 10 minutes. Pd(PPh₃)₄ (2.0 g, 3.5 mmol, 0.05 equiv.) was added and the reaction was heated to 100° C. for 16 hours at which point it was directly concentrated under vacuum. The crude residue was purified by column chromatography (SiO₂, 0-100% EtOAc in hexanes) to afford the desired product.

Step g: The product from step f (500 mg, 2.0 mmol, 1.0 equiv.) was dissolved in 7M NH₃ in MeOH (10 mL) and the resulting solution was stirred at room temperature for 4 hours. The reaction mixture was directly concentrated under vacuum and the crude residue was used without further purification.

Step h: To a suspension of the product from step g (98 mg, 0.40 mmol, 1.5 equiv.), 4-(2-chloro-6-cyclopropylpyridin-4-yl)-3-(3,3-difluoroazetidine-1-carbonyl)benzonitrile (100 mg, 0.27 mmol, 1.0 equiv.), and K₂CO₃ (111 mg, 0.80 mmol, 3.0 equiv.) in dioxane (5.5 mL) was added CuI (51 mg, 0.27 mmol, 1.0 equiv.) and DMEDA (60 uL, 0.54 mmol, 2.0 equiv.). The reaction mixture was heated to 110° C. and stirred for 16 hours at which point it was quenched with a 1:1 mixture of water/brine (100 mL) and extracted with EtOAc (2×50 mL). The combined organics were dried over Na₂SO₄, filtered, and concentrated under vacuum. The crude residue was purified by column chromatography (SiO₂, EtOAc in hexanes, 0 to 100%) to afford the desired product.

Step i: To a solution of the product from step h (88 mg, 0.15 mmol, 1.0 equiv.) in a 1:1 mixture of THF/H₂O (3 mL) was added 2,6-lutidene (35 uL, 0.30 mmol, 2.0 equiv.) followed by NaIO₄ (128 mg, 0.60 mmol, 4.0 equiv.) and K₂OsO₄*2H₂O (8 mg, 0.02 mmol, 0.1 equiv.). The reaction mixture was stirred for 2 hours at room temperature at which point it was quenched with saturated aqueous Na₂S₂O₃ (20 mL) and extracted with EtOAc (2×10 mL). The combined organics were dried over Na₂SO₄, filtered, and concentrated under vacuum. The crude residue was used directly without further purification.

Step j: To a solution of the product from step i (30 mg, 0.05 mmol, 1.0 equiv.) in DCM (1.0 mL) was added [(2S)-oxolan-2-yl]methanamine (10 mg, 0.1 mmol, 2.0 equiv.) followed by sodium triacetoxyborohydride (30 mg, 0.13 mmol, 2.5 equiv.) and AcOH (8 uL, 0.13 mmol, 2.5 equiv.). The reaction mixture was stirred for 16 hours at room temperature at which point it was quenched with saturated aqueous NaHCO₃ (20 mL) and extracted with EtOAc (2×10 mL). The combined organics were dried over Na₂SO₄, filtered, and concentrated under vacuum. The crude residue was purified by reverse phase prep-HPLC (C18 column, 5 to 60% MeCN/H₂O) to afford the desired product. ¹H NMR (400 MHz, Chloroform-d) δ 12.00 (s, 1H), 8.62 (d, J=2.6 Hz, 1H), 8.25 (d, J=1.4 Hz, 1H), 7.85-7.78 (m, 2H), 7.70-7.62 (m, 2H), 7.00 (d, J=1.5 Hz, 1H), 4.76 (q, J=8.4 Hz, 2H), 4.37 (t, J=11.8 Hz, 2H), 4.07-3.95 (m, 3H), 3.89-3.82 (m, 1H), 3.80-3.74 (m, 1H), 3.73 (s, 2H), 2.75 (dd, J=12.0, 3.4 Hz, 1H), 2.64 (dd, J=12.0, 7.8 Hz, 1H), 2.08-1.86 (m, 4H), 1.61-1.51 (m, 1H), 1.13-1.06 (m, 2H), 1.02-0.93 (m, 2H). ESI MS [M+H]⁺ for C₃₃H₃₁F₅N₆O₄ calcd 671.2 found 671.3.

### Example 94: N-[4-[4-cyano-2-(3,3-difluoroazetidine-1-carbonyl)phenyl]-6-cyclopropylpyridin-2-yl]-1-methyl-2-oxo-5-[[[(2S)-oxolan-2-yl]methylamino]methyl]pyridine-3-carboxamide

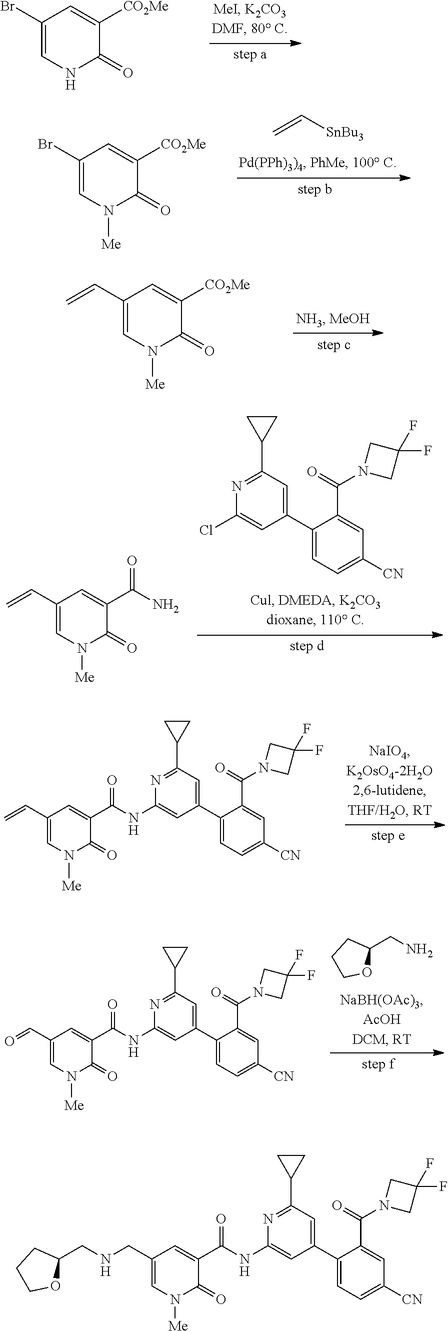

Step a: To a suspension of methyl 5-bromo-2-oxo-1H-pyridine-3-carboxylate (5.0 g, 21.6 mmol, 1.0 equiv.) and K₂CO₃ (8.9 g, 64.7 mmol, 3.0 equiv.) in DMF (100 mL) was added MeI (2.0 mL, 32.3 mmol, 1.5 equiv.). The reaction mixture was heated to 80° C. and stirred for 1 hour. The reaction was cooled to room temperature and partitioned between EtOAc (250 mL) and water (500 mL). The aqueous layer was extracted with EtOAc (2×100 mL) and the combined organics were dried over Na₂SO₄, filtered, and concentrated under vacuum. The crude residue was purified by column chromatography (SiO₂, 50-100% EtOAc in hexanes) to afford the desired product.

Step b: To a suspension of the product from step a (2.7 g, 11.0 mmol, 1.0 equiv.) in PhMe (100 mL) was added tributyl(ethenyl)stannane (4.8 mL, 16.5 mmol, 1.5 equiv.). The resulting mixture was sparged with N₂ for 10 minutes. Pd(PPh₃)₄ (630 mg, 1.1 mmol, 0.05 equiv.) was added and the reaction was heated to 100° C. for 16 hours at which point it was cooled to room temperature and directly concentrated under vacuum. The crude residue was purified by column chromatography (SiO₂, 0-100% EtOAc in hexanes) to afford the desired product.

Step c: The product from step b (729 mg, 3.8 mmol, 1.0 equiv.) was dissolved in 7M NH₃ in MeOH (15 mL) and the resulting solution was stirred at room temperature for 4 hours. The reaction mixture was directly concentrated under vacuum and the crude residue was used without further purification.

Step d: To a suspension of the product from step c (200 mg, 1.12 mmol, 1.0 equiv.), [2-(2-chloro-6-cyclopropylpyridin-4-yl)-5-fluorophenyl]-(3,3-difluoroazetidin-1-yl)methanone (prepared according to Example 93), 419 mg, 1.12 mmol, 1.0 equiv.), and K₂CO₃ (464 mg, 3.36 mmol, 3.0 equiv.) in dioxane (20 mL) was added CuI (215 mg, 1.12 mmol, 1.0 equiv.) and DMEDA (250 uL, 2.24 mmol, 2.0 equiv.). The reaction mixture was heated to 110° C. and stirred for 16 hours at which point it was quenched with a 1:1 mixture of water/brine (150 mL) and extracted with EtOAc (2×100 mL). The combined organics were dried over Na₂SO₄, filtered, and concentrated under vacuum. The crude residue was purified by column chromatography (SiO₂, EtOAc in hexanes, 0 to 100%) to afford the desired product.

Step e: To a solution of the product from step d (253 mg, 0.49 mmol, 1.0 equiv.) in a 1:1 mixture of THF/H₂O (10 mL) was added 2,6-lutidene (120 uL, 0.98 mmol, 2.0 equiv.) followed by NaIO₄ (419 mg, 1.98 mmol, 4.0 equiv.) and K₂OsO₄*2H₂O (18 mg, 0.05 mmol, 0.1 equiv.). The reaction mixture was stirred for 16 hours at room temperature at which point it was quenched with saturated aqueous Na₂S₂O₃ (100 mL) and extracted with EtOAc (2×100 mL). The combined organics were dried over Na₂SO₄, filtered, and concentrated under vacuum. The crude residue was used directly without further purification.

Step f: To a solution of the product from step e (35 mg, 0.07 mmol, 1.0 equiv.) in DCM (1.5 mL) was added [(2S)-oxolan-2-yl]methanamine (14 mg, 0.14 mmol, 2.0 equiv.) followed by sodium triacetoxyborohydride (38 mg, 0.17 mmol, 2.5 equiv.) and AcOH (10 uL, 0.17 mmol, 2.5 equiv.). The reaction mixture was stirred for 1 hour at room temperature at which point it was quenched with saturated aqueous NaHCO₃ (20 mL) and extracted with EtOAc (2×10 mL). The combined organics were dried over Na₂SO₄, filtered, and concentrated under vacuum. The crude residue was purified by reverse phase prep-HPLC (C18 column, 5 to 60% MeCN/H₂O) to afford the desired product. ¹H NMR (400 MHz, Chloroform-d) δ 12.38 (s, 1H), 8.55 (d, J=2.6 Hz, 1H), 8.26 (d, J=1.4 Hz, 1H), 7.84-7.78 (m, 2H), 7.70-7.64 (m, 2H), 6.99 (d, J=1.5 Hz, 1H), 4.36 (t, J=11.8 Hz, 2H), 4.05-3.94 (m, 3H), 3.90-3.82 (m, 1H), 3.80-3.72 (m, 1H), 3.70 (s, 5H), 2.73 (dd, J=12.0, 3.5 Hz, 1H), 2.64 (dd, J=12.0, 7.8 Hz, 1H), 2.08-1.84 (m, 4H), 1.15-1.07 (m, 2H), 1.00-0.93 (m, 2H). ESI MS [M+H]⁺ for C₃₂H₃₂F₂N₆O₄ calcd 603.3 found 603.3.

### Example 95: N-(4-{4-cyano-2-[(3,3-difluoro-1-azetidinyl)carbonyl]phenyl}-6-cyclopropyl-2-pyridyl)-5-{[(S)-2-methoxypropylamino]methyl}-1-cyclopropyl-2-oxo-1,2-dihydronicotinamide

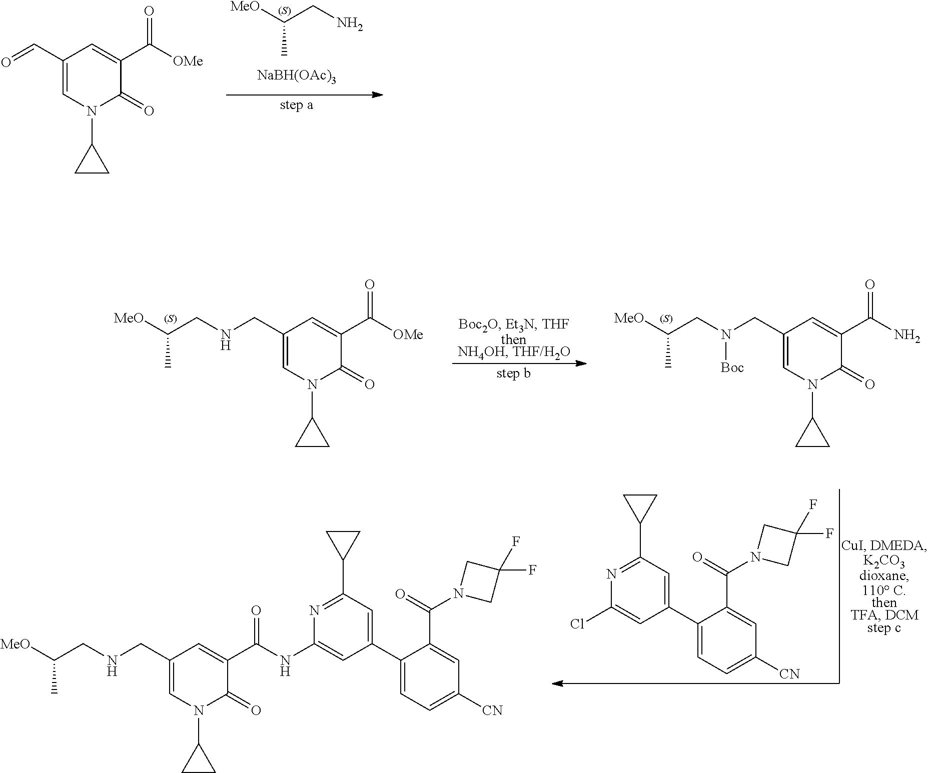

Step a: To a solution of methyl 1-cyclopropyl-5-formyl-2-oxopyridine-3-carboxylate (4.0 g, 18.09 mmol, 1.0 equiv.) in dichloromethane (90 mL, 0.2 M) was added 2-methoxyethanamine (1.9 g, 21.71 mmol, 1.2 equiv.) and AcOH (2.06 mL 36.18 mmol, 2.0 equiv.) and the mixture was stirred at 23° C. for 30 mins. Then, NaBH(OAc)₃ (7.6 g, 36.18 mmol, 2.0 equiv.) was added, and the mixture was stirred at 23° C. overnight. The reaction was quenched with a slow addition of aq. Sat. NaHCO₃ (200 mL) and diluted with 3:1 mixture of CHCl₃/iPrOH (300 mL). The organic phase was separated, and the aqueous layer was additionally extracted with CHCl₃/iPrOH (2×100 mL). The combined organic extract was dried over Na₂SO₄ and concentrated to dryness under reduced pressure. The crude residue was purified by column chromatography (SiO₂, 0-25% MeOH in dichloromethane) to afford the desired product.

Step b: To the solution of the product from step a (5.6 g, 19.04 mmol, 1.0 equiv.) in THF (65 mL, 0.3 M) was added triethylamine (7.9 mL, 57.12 mmol, 3.0 equiv.) and Boc₂O (5.4 g, 24.75 mmol, 1.3 equiv.) The resulting mixture was stirred at 23° C. overnight. Then, the mixture was concentrated to dryness under a vacuum. The crude residue was purified by reversed-phase column chromatography (C18, 10-100% CH₃CN in water with 0.1% formic acid) to afford the desired product. The resulting product was dissolved in 7 N NH₃ in MeOH and stirred overnight. After completion, the reaction mixture was concentrated to dryness under reduced pressure to afford the tert-butyl N-[(5-carbamoyl-1-cyclopropyl-6-oxopyridin-3-yl)methyl]-N-[(2S)-2-methoxypropyl]carbamate as the desired product.

Step c: To a solution of the product from step b (2.2 g, 5.80 mmol, 1.0 equiv.) and 4-(2-chloro-6-cyclopropylpyridin-4-yl)-3-(3,3-difluoroazetidine-1-carbonyl)benzonitrile (2.3 g, 6.38 mmol, 1.1 equiv., prepared according to Example 93) in dioxane (116 mL, 0.05 M) were added CuI (1.10 g, 5.80 mmol, 1.0 equiv.), DMEDA (1.2 mL, 11.6 mmol, 2.0 equiv.) and K₂CO₃ (2.4 g, 17. mmol, 3.0 equiv.). The resulting mixture was degassed under vacuum and backfilled with nitrogen (repeated 3 times). The resulting solution was stirred at 110° C. for 24 h. The mixture was cooled to room temperature, diluted with EtOAc (250 mL), aq. sat NH₄Cl (50 mL) and water (50 mL). The organic phase was separated, and the aqueous layer was extracted with EtOAc ((2×100 mL). The combined organic phase was dried over Na₂SO₄, concentrated to dryness, and the crude residue was purified by column chromatography (SiO₂, 0-20% MeOH in dichloromethane) to yield the desired product. The obtained product was dissolved in a 3:1 mixture of dichloromethane (30 mL) and TFA (10 ml). After stirring for 2 h, the solution was concentrated to dryness. The crude residue was purified by reversed-phase column chromatography (C18, 10-100% CH₃CN in water with 0.1% formic acid) to afford the title compound. ¹H NMR (400 MHz, Chloroform-d) δ 12.34 (s, 1H), 8.55 (d, J=2.5 Hz, 1H), 8.27 (s, 1H), 7.85-7.77 (m, 2H), 7.71-7.61 (m, 2H), 6.99 (s, 1H), 4.36 (t, J=11.8 Hz, 2H), 3.99 (t, J=11.8 Hz, 2H), 3.70-3.60 (m, 2H), 3.52-3.43 (m, 2H), 3.35 (s, 3H), 2.66-2.58 (m, 2H), 2.07-1.97 (m, 1H), 1.30-1.22 (m, 2H), 1.15 (d, J=6.2 Hz, 3H), 1.13-1.09 (m, 2H), 1.01-0.93 (m, 4H). ESI MS [M+H]⁺ for C₃₃H₃₄F₂N₆O₄, calcd 617.2, found 617.2.

### Example 96: N-(4-{4-cyano-2-[(3,3-difluoro-1-azetidinyl)carbonyl]phenyl}-6-isopropoxy-2-pyridyl)-5-[({[(S)-tetrahydro-2-furyl]methyl}amino)methyl]-1-cyclopropyl-2-oxo-1,2-dihydronicotinamide

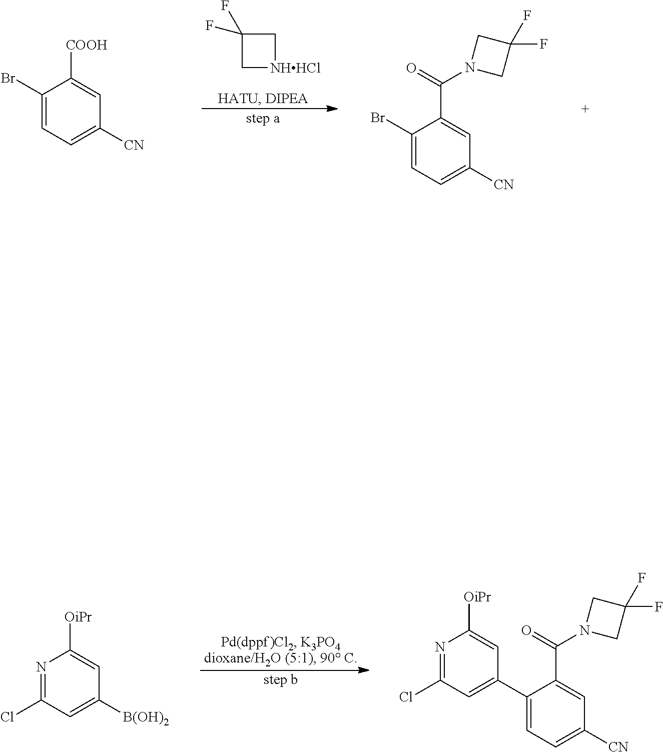

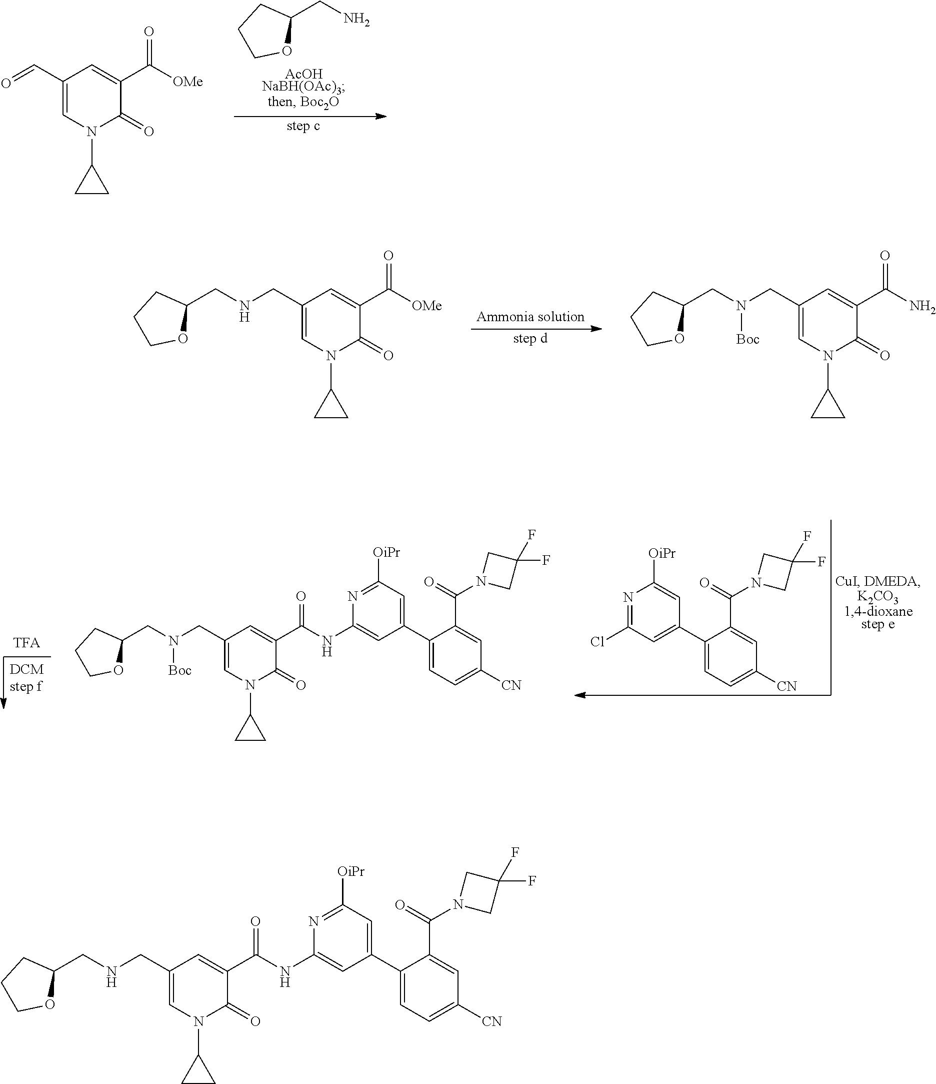

Step a: To a solution of a 2-bromo-5-cyanobenzoic acid (10.0 g, 44.24 mmol, 1 equiv.) in DCM (200 mL, 0.2 M) was added i-Pr₂NEt (12.4 mL, 88.48 mmol, 2 equiv.), HATU (25.2 g, 66.36 mmol, 1.5 equiv.) and 3,3-difluoroazetidine hydrochloride (6.88 g, 53.1 mmol, 1.2 equiv.). The reaction mixture was stirred overnight at room temperature and diluted with EtOAc and water. The organic phase was separated, washed with brine, dried over Na₂SO₄ and concentrated to dryness under reduced pressure. The crude residue was purified by silica gel flash column chromatography (SiO₂, hextane in EtOAc, 0 to 60%) to afford 4-bromo-3-(3,3-difluoroazetidine-1-carbonyl)benzonitrile.

Step b: To a solution of the product of step a (10.55 g, 35.04 mmol, 1.0 equiv.), (2-chloro-6-propan-2-yloxypyridin-4-yl)boronic acid (7.55 g, 35.04 mmol, 1.0 equiv.) and K₃PO₄ (22.3 g, 105.12 mmol, 3.0 equiv.) in dioxane (200 mL, 0.15 M) and H₂O (40 mL, 0.15 M) was added Pd(dppf)Cl₂ (2.56 g, 3.504 mmol, 0.1 equiv.) at rt under nitrogen atmosphere. The resulting mixture was stirred for 1 h at 90° C. The mixture was allowed to cool down to rt. The reaction was quenched with sat. aq. NH₄Cl solution, the organic phase was separated, and the aqueous layer was extracted with EtOAc. The combined organic phase was dried over Na₂SO₄, concentrated and the crude residue was purified by column chromatography (SiO₂, hexane in EtOAc, 0 to 60%) to afford 4-(2-chloro-6-propan-2-yloxypyridin-4-yl)-3-(3,3-difluoroazetidine-1-carbonyl)benzonitrile.

Step c: To the solution of 1-cyclopropyl-5-formyl-2-oxopyridine-3-carboxylate (7.08 g, 32 mmol, 1.0 equiv.) in DCM (200 mL, 0.15 M) was added [(2S)-oxolan-2-yl]methanamine (4.21 g, 41.6 mmol, 1.3 equiv.) and AcOH (2.75 ml, 48 mmol, 1.5 equiv.) and the mixture was stirred for 10 min. NaBH(OAc)₃ (13.6 g, 64 mmol, 2.0 equiv.) was added and the mixture was stirred at rt for 2 h. (Boc)₂O (10.5 g, 48 mmol, 1.5 equiv.) was added into the above solution and was stirred at rt for 12 h. The reaction mixture was diluted with DCM, washed with H₂O and brine, filtered through Na₂SO₄, and concentrated and the crude residue was purified by column chromatography (SiO₂, 0-20% MeOH gradient in DCM) to afford methyl 1-cyclopropyl-2-oxo-5-[[[(2S)-oxolan-2-yl]methylamino]methyl]pyridine-3-carboxylate.

Step d: The solution of the product from step c (10.2 g, 33.3 mmol, 1.0 equiv.) in ammonia solution (50 mL, 0.66 M) was stirred overnight. Then the mixture was concentrated to dryness under vacuum to yield tert-butyl N-[(5-carbamoyl-1-cyclopropyl-6-oxopyridin-3-yl)methyl]-N-[[(2S)-oxolan-2-yl]methyl]carbamate. The crude residue was used for the next step directly without further purification.

Step e: To a mixture of the product of step d (3.5 g, 8.94 mmol, 1.05 equiv.) and the product of step b (3.34 g, 8.52 mmol, 1.0 equiv.) in dioxane (100 mL) was added CuI (3.24 g, 17.03 mmol, 1.0 equiv.), N,N′-dimethylethylenediamine (7.32 ml, 68.12 mmol, 4.0 equiv.) and K₂CO₃ (5.88 g, 42.58 mmol, 5.0 equiv.) The resulting mixture was heated at 110° C. overnight. The reaction mixture was cooled to room temperature, diluted with EtOAc, washed with H₂O and brine, filtered through Na₂SO₄, and concentrated. The crude product was purified by column chromatography (SiO₂, 0-100% EtOAc gradient in hexanes) to produce tert-butyl N-[[5-[[4-[4-cyano-2-(3,3-difluoroazetidine-1-carbonyl)phenyl]-6-propan-2-yloxypyridin-2-yl]carbamoyl]-1-cyclopropyl-6-oxopyridin-3-yl]methyl]-N-[[(2S)-oxolan-2-yl]methyl]carbamate.

Step f: The solution of the product of step e (4.26 g, 5.7 mmol, 1.0 equiv.) was treated with TFA/DCM (v/v 1:5, 120 mL) at room temperature for 1.5 h. The mixture was concentrated under vacuum, and the crude residue was then purified by reversed-phase column chromatography (C18, 10-100% CH₃CN in water with 0.1% formic acid) to afford the title compound. ¹H NMR (400 MHz, DMSO-d₆) δ 12.54 (s, 1H), 8.46 (d, J=2.6 Hz, 1H), 8.14 (d, J=1.7 Hz, 1H), 8.06 (dd, J=8.1, 1.7 Hz, 1H), 7.91 (t, J=2.5 Hz, 2H), 7.76 (d, J=8.0 Hz, 1H), 6.58 (d, J=1.3 Hz, 1H), 5.29 (p, J=6.2 Hz, 1H), 4.41 (dt, J=28.5, 12.2 Hz, 5H), 3.92-3.80 (m, 1H), 3.72 (dt, J=8.1, 6.5 Hz, 1H), 3.64-3.56 (m, 3H), 3.51 (tt, J=7.6, 4.3 Hz, 1H), 2.13 (s, 1H), 1.96-1.71 (m, 3H), 1.60-1.45 (m, 1H), 1.34 (d, J=6.1 Hz, 6H), 1.20-1.07 (m, 2H), 1.03-0.94 (m, 2H). ESI MS [M+H]⁺ for C₃₅H₃₇F₂N₆O₅, calcd 647.3, found 647.2.

### Example 97: N-(4-{4-cyano-2-[(3,3-difluoro-1-azetidinyl)carbonyl]phenyl}-6-isopropoxy-2-pyridyl)-1-cyclopropyl-5-[(isobutylamino)methyl]-2-oxo-1,2-dihydronicotinamide

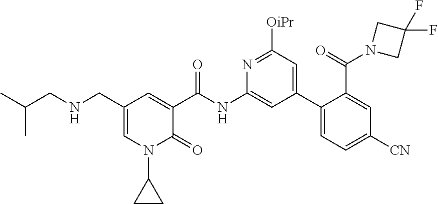 [SMILES: CC(C)CNCc1cc(C(=O)Nc2cc(-c3ccc(C#N)cc3C(=O)N3CC(F)(F)C3)cc(OC(C)C)n2)c(=O)n(C2CC2)c1]

The title compound was prepared in a similar fashion to that described for Example 95 using isobutylamine in step a. ¹H NMR (400 MHz, DMSO-d₆) δ 12.54 (s, 1H), 8.48 (d, J=2.5 Hz, 1H), 8.14 (d, J=1.7 Hz, 1H), 8.06 (dd, J=8.1, 1.7 Hz, 1H), 7.91 (d, J=1.3 Hz, 2H), 7.76 (d, J=8.1 Hz, 1H), 6.59 (s, 1H), 5.29 (p, J=6.1 Hz, 1H), 4.41 (dt, J=29.9, 12.2 Hz, 4H), 3.77-3.44 (m, 3H), 2.29 (d, J=6.7 Hz, 2H), 1.66 (dp, J=13.6, 6.7 Hz, 1H), 1.34 (d, J=6.1 Hz, 6H), 1.27-1.22 (m, 1H), 1.11 (td, J=7.4, 5.6 Hz, 2H), 1.03-0.92 (m, 2H), 0.86 (d, J=6.5 Hz, 6H). ESI MS [M+H]⁺ for C₃₃H₃₇F₂N₆O₄, calcd 619.3, found 619.2.

### Example 98: N-(4-{2-[(3,3-difluoro-1-azetidinyl)carbonyl]-4-difluoromethoxyphenyl}-6-isopropoxy-2-pyridyl)-5-{[((S)-1-cyclobutylethylamino]methyl}-1-cyclopropyl-2-oxo-1,2-dihydronicotinamide

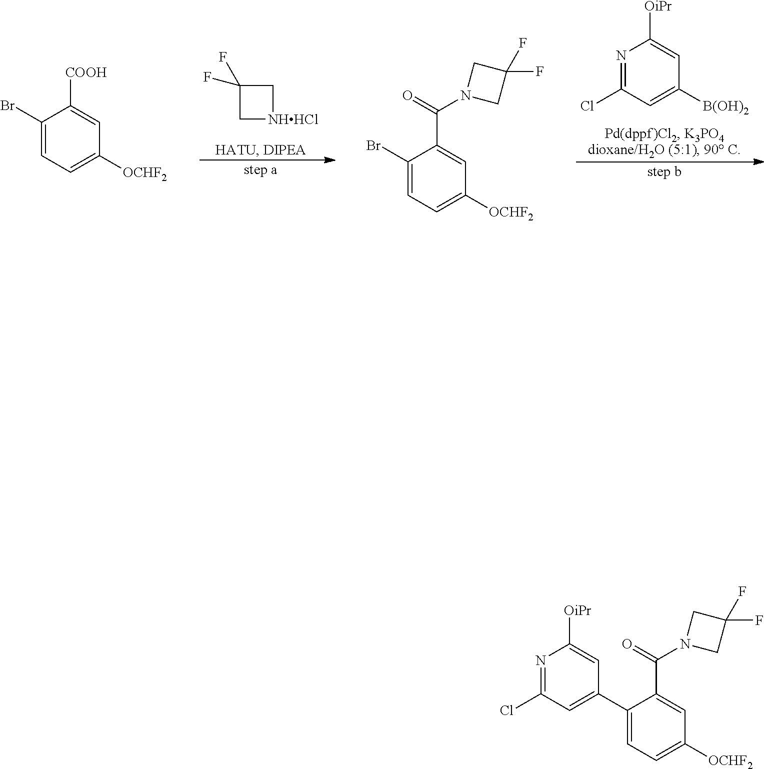

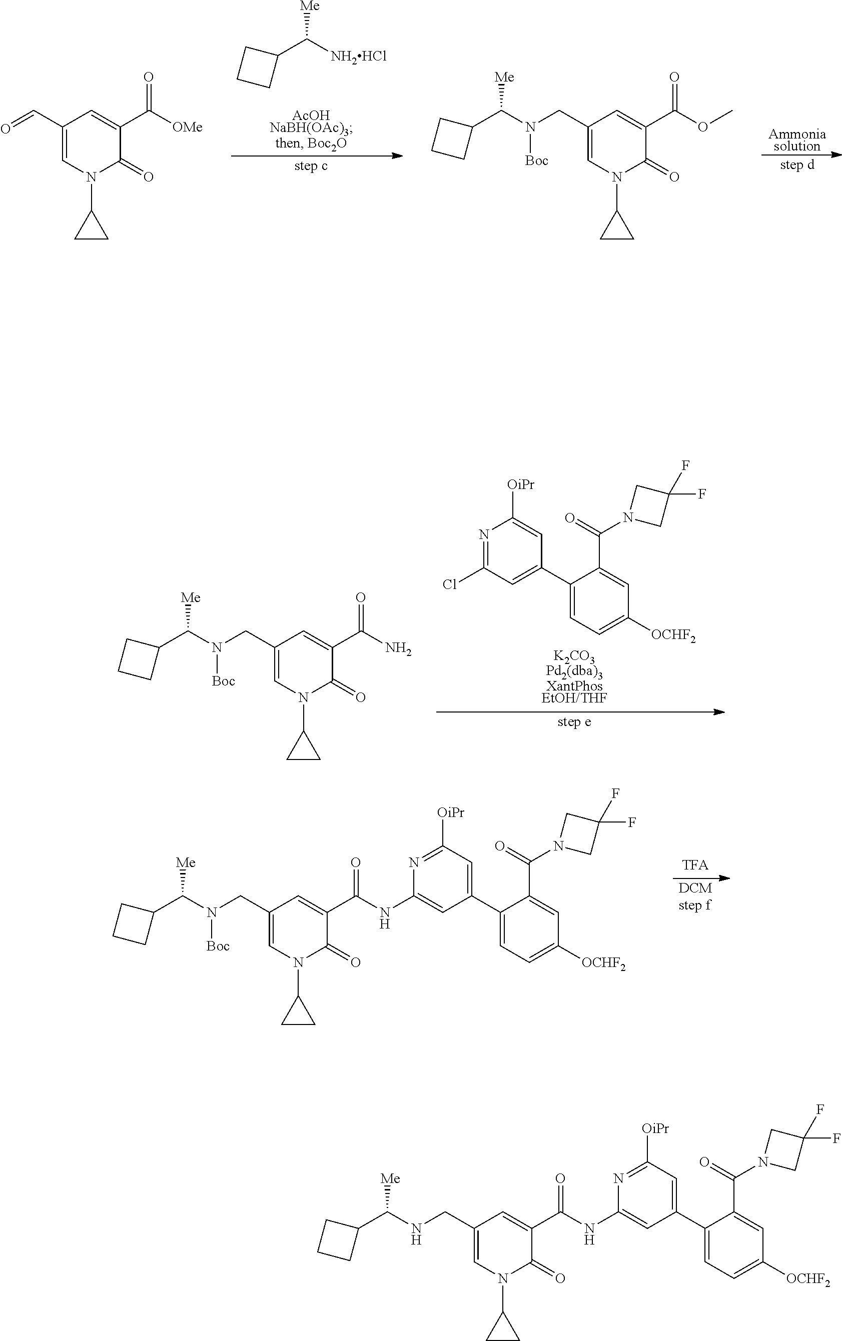

Step a: To a solution of a 2-bromo-5-(difluoromethoxy)benzoic acid (16.9 g, 63.29 mmol, 1 equiv.) in DCM (200 mL, 0.3 M) was added i-Pr₂NEt (18 mL, 126.58 mmol, 2 equiv.), HATU (36.1 g, 94.94 mmol, 2 equiv.) and 3,3-difluoroazetidine hydrochloride (9.84 g, 75.95 mmol, 1.2 equiv.). The reaction mixture was stirred overnight at room temperature and diluted with EtOAc and water. The organic phase was separated, washed with brine, dried over Na₂SO₄ and concentrated to dryness under reduced pressure. The crude residue was purified by silica gel flash column chromatography (SiO₂, hextane in EtOAc, 0 to 60%) to afford [2-bromo-5-(difluoromethoxy)phenyl]-(3,3-difluoroazetidin-1-yl)methanone.

Step b: To a solution of the product of step a (16.68 g, 44.24 mmol, 1.0 equiv.), (2-chloro-6-propan-2-yloxypyridin-4-yl)boronic acid (10.5 g, 48.76 mmol, 1.0 equiv.) and K₃PO₄ (31.05 g, 146.28 mmol, 3.0 equiv.) in dioxane (250 mL, 0.15 M) and H₂O (50 mL, 0.15 M) was added Pd(dppf)Cl₂ (3.6 g, 4.876 mmol, 0.1 equiv.) at rt under nitrogen atmosphere. The resulting mixture was stirred for 1 h at 90° C. The mixture was allowed to cool down to rt. The reaction was quenched with sat. aq. NH₄Cl solution, the organic phase was separated, and the aqueous layer was extracted with EtOAc. The combined organic phase was dried over Na₂SO₄, concentrated and the crude residue was purified by column chromatography (SiO₂, hextane in EtOAc, 0 to 60%) to afford [2-(2-chloro-6-propan-2-yloxypyridin-4-yl)-5-(difluoromethoxy)phenyl]-(3,3-difluoroazetidin-1-yl)methanone.

Step c: To the solution of 1-cyclopropyl-5-formyl-2-oxopyridine-3-carboxylate (6.8 g, 30.7 mmol, 1.0 equiv.) in DCM (200 mL, 0.15 M) was added (1S)-1-cyclobutylethan-1-amine hydrochloride (5.0 g, 36.8 mmol, 1.2 equiv.) and AcOH (3.5 ml, 46.1 mmol, 1.5 equiv.) and the mixture was stirred for 10 min. NaBH(OAc)₃ (13 g, 61.4 mmol, 2.0 equiv.) was added and the mixture was stirred at rt for 2 h. (Boc)₂O (10.1 g, 48 mmol, 1.5 equiv.) was added into the above solution and was stirred at rt for 12 h. The reaction mixture was diluted with DCM, washed with H₂O and brine, filtered through Na₂SO₄, and concentrated and the crude residue was purified by column chromatography (SiO₂, 0-20% MeOH gradient in DCM) to afford methyl 5-[[[(1S)-1-cyclobutylethyl]-[(2-methylpropan-2-yl)oxycarbonyl]amino]methyl]-1-cyclopropyl-2-oxopyridine-3-carboxylate.

Step d: The solution of the product from step c (8 g, 16.34 mmol, 1.0 equiv.) in ammonia solution (40 mL, 0.4 M) was stirred overnight. Then the mixture was concentrated to dryness under vacuum to yield tert-butyl N-[(5-carbamoyl-1-cyclopropyl-6-oxopyridin-3-yl)methyl]-N-[(1S)-1-cyclobutylethyl]carbamate. The crude residue was used for the next step directly without further purification.

Step e: To a mixture of the product of step d (1.88 g, 4.83 mmol, 1.06 equiv.) and the product of step b (1.97 g, 4.55 mmol, 1.0 equiv.) in EtOH (20 mL) was added K₂CO₃ (723 mg, 5.24 mmol, 1.15 equiv.) and the mixture stirred for 10 min at rt to obtain a white suspension. Pd₂(dba)₃ (20.8 mg, 0.023 mmol, 0.005 equiv.) and XantPhos (26.4 mg, 0.046 mmol, 0.01 equiv.) were dissolved in THF (5 ml) and stirred for 10 min at rt to obtain a red mixture. The red mixture was added to the white suspension, and the mixture turned yellow-orange. The resulting mixture was heated at 85° C. overnight. The reaction mixture was cooled to room temperature, diluted with EtOAc, washed with H₂O and brine, filtered through Na₂SO₄, and concentrated. The crude product was purified by column chromatography (SiO₂, 0-100% EtOAc gradient in hexanes) to produce tert-butyl N-[(1S)-1-cyclobutylethyl]-N-[[1-cyclopropyl-5-[[4-[2-(3,3-difluoroazetidine-1-carbonyl)-4-(difluoromethoxy)phenyl]-6-propan-2-yloxypyridin-2-yl]carbamoyl]-6-oxopyridin-3-yl]methyl]carbamate.

Step f: A solution of the product of step e (3 g, 3.8 mmol, 1.0 equiv.) was treated with TFA/DCM (v/v 1:5, 120 mL) at room temperature for 1.5 h. The mixture was concentrated under vacuum, and the crude residue was then purified by reversed-phase column chromatography (C18, 10-100% CH₃CN in water with 0.1% formic acid) to afford the title compound. ¹H NMR (400 MHz, DMSO-d₆) δ 12.51 (s, 1H), 8.49 (d, J=2.5 Hz, 1H), 7.91 (dd, J=6.6, 1.9 Hz, 2H), 7.73-7.54 (m, 1H), 7.39 (m, 3H), 6.52 (d, J=1.3 Hz, 1H), 5.28 (p, J=6.2 Hz, 1H), 4.34 (q, J=11.4 Hz, 4H), 3.80-3.46 (m, 4H), 2.44 (t, J=7.2 Hz, 1H), 2.20-2.10 (m, 1H), 1.97 (ddd, J=10.0, 7.4, 3.3 Hz, 1H), 1.87 (tq, J=7.8, 3.9 Hz, 1H), 1.81-1.61 (m, 3H), 1.33 (d, J=6.1 Hz, 6H), 1.21-1.07 (m, 2H), 1.05-0.94 (m, 2H), 0.88 (d, J=6.2 Hz, 3H). ESI MS [M+H]⁺ for C₃₅H₄₀F₄N₅O₅, calcd 686.3, found 686.2.

### Example 99: N-(4-{2-[(3,3-difluoro-1-azetidinyl)carbonyl]-4-difluoromethoxyphenyl}-6-isopropoxy-2-pyridyl)-1-cyclopropyl-5-[(isobutylamino)methyl]-2-oxo-1,2-dihydronicotinamide

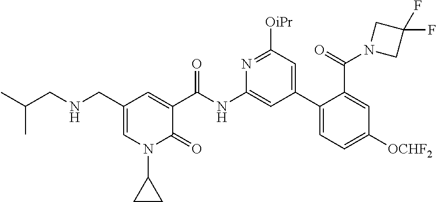 [SMILES: CC(C)CNCc1cc(C(=O)Nc2cc(-c3ccc(OC(F)F)cc3C(=O)N3CC(F)(F)C3)cc(OC(C)C)n2)c(=O)n(C2CC2)c1]

The title compound was prepared in a similar fashion to that described for Example 98 using isobutylamine in step c. ¹H NMR (400 MHz, DMSO-d₆) δ 12.51 (s, 1H), 8.48 (d, J=2.5 Hz, 1H), 7.90 (t, J=2.2 Hz, 2H), 7.63 (d, J=9.3 Hz, 1H), 7.59-7.03 (m, 3H), 6.52 (d, J=1.3 Hz, 1H), 5.28 (p, J=6.2 Hz, 1H), 4.34 (q, J=11.5 Hz, 4H), 3.67-3.45 (m, 3H), 2.29 (d, J=6.7 Hz, 2H), 1.66 (dt, J=13.3, 6.6 Hz, 1H), 1.33 (d, J=6.2 Hz, 6H), 1.11 (td, J=7.5, 5.4 Hz, 2H), 1.03-0.96 (m, 2H), 0.86 (d, J=6.6 Hz, 6H). ESI MS [M+H]⁺ for C₃₃H₃₈F₄N₅O₅, calcd 660.3, found 660.2.

### Example 100: N-[4-[4-cyano-2-(3,3-difluoroazetidine-1-carbonyl)phenyl]-6-propan-2-yloxypyridin-2-yl]-5-[[[(1S)-1-cyclobutylethyl]amino]methyl]-1-methyl-2-oxopyridine-3-carboxamide

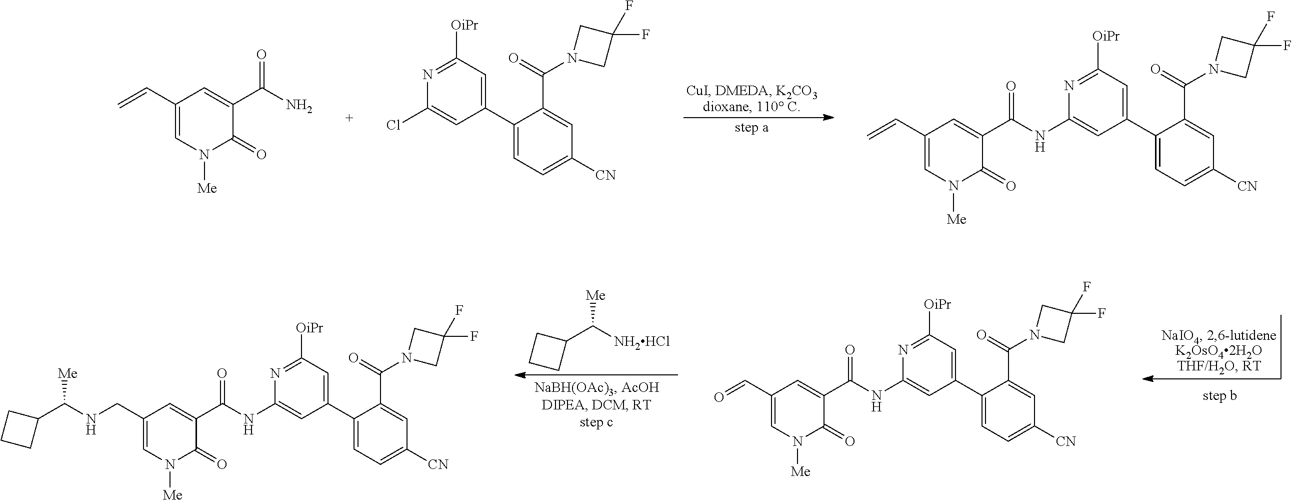

Step a: To a suspension of 5-ethenyl-1-methyl-2-oxopyridine-3-carboxamide (191 mg, 1.1 mmol, 1.0 equiv.) (prepared according to Example 94), 4-(2-chloro-6-propan-2-yloxypyridin-4-yl)-3-(3,3-difluoroazetidine-1-carbonyl)benzonitrile (419 mg, 1.1 mmol, 1.0 equiv., prepared according to Example 96), and K₂CO₃ (886 mg, 6.4 mmol, 6.0 equiv.) in dioxane (21.4 mL) was added CuI (407 mg, 2.1 mmol, 2.0 equiv.) and DMEDA (377 mg, 4.3 mmol, 4.0 equiv.). The reaction mixture was heated to 110° C. and stirred for 90 hours at which point it was quenched with a 1:1 mixture of saturated NH₄Cl solution (20 mL) and extracted with EtOAc (2×10 mL). The combined organics were dried over Na₂SO₄, filtered, and concentrated under vacuum. The crude residue was purified by column chromatography (SiO₂, EtOAc in hexanes, 0 to 100%) to afford the desired product.

Step b: To a solution of the product from step a (332 mg, 0.62 mmol, 1.0 equiv.) in a mixture of THF (6 mL) and H₂O (1.5 mL) was added 2,6-lutidene (0.15 mL, 1.2 mmol, 2.0 equiv.) followed by NaIO₄ (530 mg, 2.5 mmol, 4.0 equiv.) and K₂OsO₄·2H₂O (46 mg, 0.12 mmol, 0.2 equiv.). The reaction mixture was stirred for 16 hours at room temperature at which point it was quenched with saturated aqueous Na₂S₂O₃ (10 mL) and extracted with EtOAc (2×10 mL). The combined organics were dried over Na₂SO₄, filtered, and concentrated under vacuum. The crude residue was used directly without further purification.

Step c: To a solution of the product from step b (53.5 mg, 0.1 mmol, 1.0 equiv.) in DCM (1.0 mL) was added (1S)-1-cyclobutylethanamine hydrochloride (27.0 mg, 0.2 mmol, 2.0 equiv.) followed by sodium triacetoxyborohydride (64 mg, 0.3 mmol, 3.0 equiv.), DIPEA (70 μL, 0.4 mmol, 2.0 equiv.) and AcOH (12 μL, 0.2 mmol, 2.0 equiv.). The reaction mixture was stirred for 16 hours at room temperature at which point it was quenched with H₂O (2 mL) and extracted with DCM (2×2 mL). The combined organics were dried over Na₂SO₄, filtered, and concentrated under vacuum. The crude residue was purified by reverse phase prep-HPLC (C18 column, 5 to 60% MeCN/H₂O) to afford the desired product. ¹H NMR (400 MHz, Chloroform-d) δ 12.31 (s, 1H), 8.57 (d, J=2.6 Hz, 1H), 8.04 (d, J=1.3 Hz, 1H), 7.82-7.77 (m, 2H), 7.70-7.64 (m, 2H), 6.54 (d, J=1.3 Hz, 1H), 5.43 (p, J=6.2 Hz, 1H), 4.38 (t, J=11.9 Hz, 2H), 4.04 (t, J=11.5 Hz, 2H), 3.77-3.67 (m, 4H), 3.60 (d, J=13.6 Hz, 1H), 2.65-2.54 (m, 1H), 2.25 (q, J=8.2 Hz, 1H), 2.09-1.92 (m, 1H), 1.86 (t, J=8.1 Hz, 1H), 1.81-1.63 (m, 5H), 1.34 (d, J=6.1 Hz, 6H), 0.99 (d, J=6.2 Hz, 3H). ESI MS [M+H]⁺ for C₃₃H₃₇F₂N₆O₄ calcd. 619.3 found 619.3.

### Example 101: N-[4-[4-cyano-2-(3,3-difluoroazetidine-1-carbonyl)-6-fluorophenyl]-6-propan-2-yloxypyridin-2-yl]-1-cyclopropyl-2-oxo-5-[[[(2S)-oxolan-2-yl]methylamino]methyl]pyridine-3-carboxamide

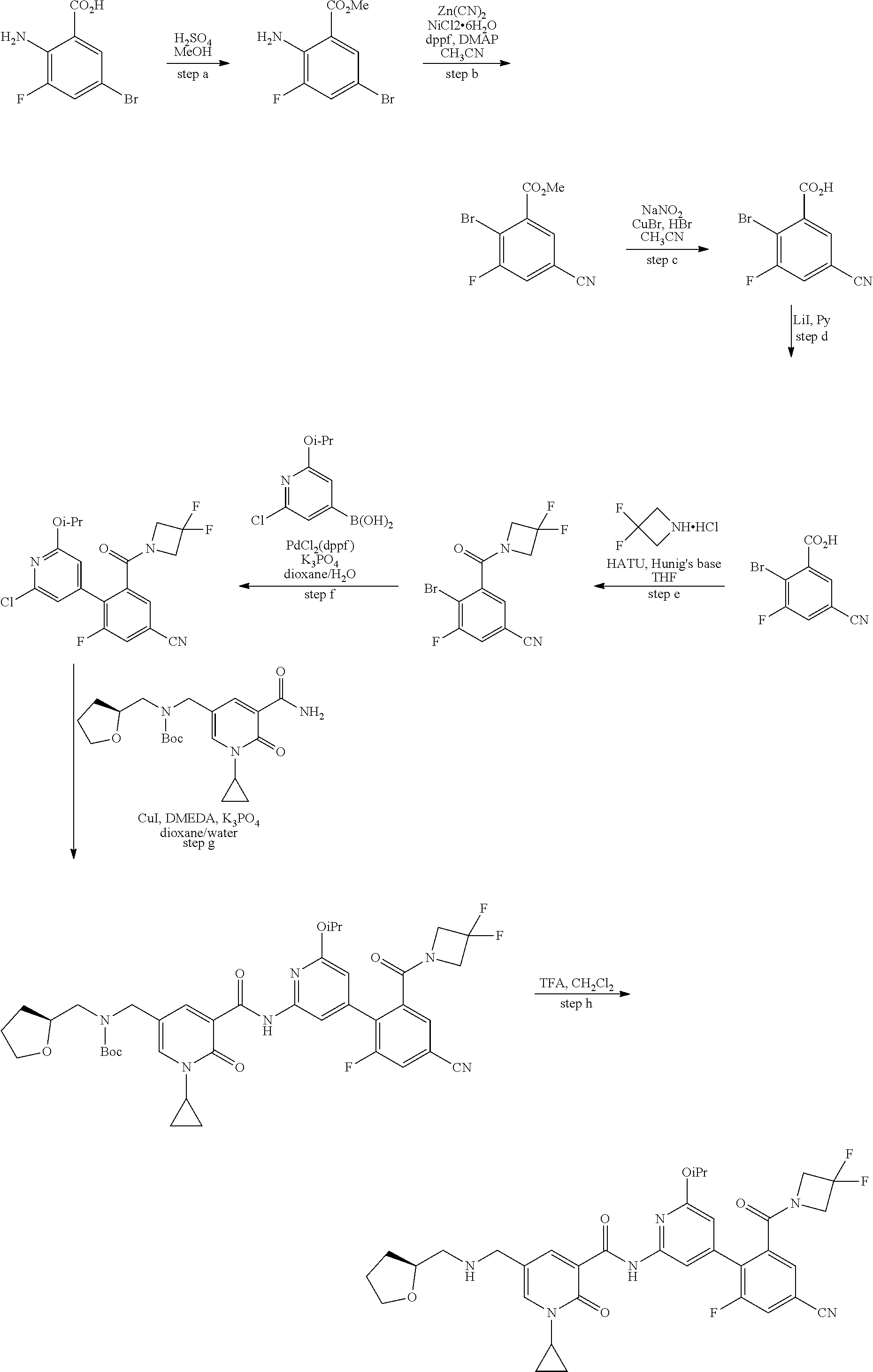

Step a: 2-Amino-5-bromo-3-fluorobenzoic acid (3.0 g, 12.8 mmol, 1 equiv.) was dissolved in an anhydrous methanol (12.5 mL) and concentrated sulfuric acid (0.5 mL) was added dropwise over 3 min. period. The resulting mixture was refluxed for 3 h. Once TLC analysis indicated complete consumption of the starting material the reaction mixture was cooled to room temperature. The obtained solution was diluted with water and basified with aq. Na₂CO₃ (2 M) to pH˜8. The formed precipitate was collected by filtration and washed with water (3×30 mL). The product was dried on filter under vacuum for 24 h to afford methyl 2-amino-5-bromo-3-fluorobenzoate with sufficient purity for the next step.

Step b: The product of step a (14.9 g, 0.06 mol, 1 equiv.), Zn(CN)₂ (7.0 g, 0.06 mol, 1 equiv.), NiCl₂·6H₂O (1.4 g, 6 mmol, 0.1 equiv.), dppf (4.0 g, 7.2 mmol, 0.12 equiv.), DMAP (7.3 g, 0.06 mol, 1 equiv.) and Zn (1.2 g, 0.02 mol, 0.3 equiv., powder preactivated by sequential wash with 1 M aq. HCl, acetone and dry diethyl ether) were suspended in CH₃CN (300 mL). The resulting mixture was degassed under vacuum and backfilled with nitrogen (repeated 3 times) and refluxed under vigorous stirring for 24 h. The obtained dark orange mixture was cooled to room temperature and partitioned between water (400 mL) and EtOAc (500 mL). The obtained heterogenous mixture was filtered through CELITE®, the organic phase was separated, and the aqueous phase was additionally extracted with EtOAc (2×100 ml). The combined organic extract was dried over Na₂SO₄ and concentrated to dryness under reduced pressure. The crude product was purified by column chromatography (SiO₂, 0-60% EtOAc gradient in hexanes) to produce methyl 2-amino-5-cyano-3-fluorobenzoate.

Step c: To a solution of the product of step b (9.3 g, 0.05 mol, 1 equiv.) in CH₃CN (250 mL) was added aq. HBr (48% solution, 0.2 mol, 4 equiv.) at 0° C. The reaction mixture was stirred for 10 min before NaNO₂ (4.8 g, 0.07 mol, 1.3 equiv.) solution in water (35 mL) was added in portions over 15 min at 0° C. The obtained mixture was stirred for 30 min, during which time the initially formed hydrobromide of the aniline completely dissolved and the corresponding diazonium salt began to precipitate. CuBr (8.6 g, 0.06 mol, 1.2 equiv.) was added in portions over 5 min at 0° C., then the cooling bath was removed, and the reaction mixture was stirred for 24 h at room temperature. The obtained dark solution was diluted with EtOAc (300 mL) and washed with NH₄Cl (250 mL). The aqueous phase was separated and additionally extracted with EtOAc (2×100 mL). The combined organic extract was dried over Na₂SO₄ and concentrated to dryness under reduced pressure. The crude product was purified by column chromatography (SiO₂, 0-60% EtOAc gradient in hexanes) to produce methyl 2-bromo-5-cyano-3-fluorobenzoate.

Step d: A mixture of the product from step c (8.7 g, 34 mmol, 1 equiv.) and LiI (22.6 g, 0.17 mol, 5 equiv.) in pyridine (170 ml, 0.2 M) was refluxed for 3 h. TLC and LCMS analysis indicated complete transformation of the starting material. The obtained solution was cooled to room temperature and concentrated to ˜40 mL. This material was partitioned between EtOAc (250 mL) and aq. HCl solution (1 M, 300 mL). The organic phase was separated, and the aqueous phase was additionally extracted with EtOAc (2×70 mL). The combined organic extract was dried over Na₂SO₄ and concentrated to dryness under reduced pressure to afford crude 2-bromo-5-cyano-3-fluorobenzoic acid that was used for the next step without purification.

Step e: The crude carboxylic acid from step d (approx. 34 mmol, 1 equiv.) was dissolved in THF (70 mL) followed by the sequential addition of 3,3-difluoroazetidine hydrochloride (5.2 g, 40.0 mmol, 1.2 equiv.), Hunig's base (12.2 mL, 70 mmol, 2 equiv.) and HATU (15.2 g, 40 mmol, 1.2 equiv.). The resulting suspension was stirred at room temperature for 24 h, then it was concentrated to dryness with CELITE® and directly fractionated by column chromatography (SiO₂, 0-80% EtOAc gradient in hexanes) to afford 4-bromo-3-(3,3-difluoroazetidine-1-carbonyl)-5-fluorobenzonitrile.

Step f: A mixture of the product from step e (5.65 g, 18.0 mmol, 1 equiv.), (2-chloro-6-propan-2-yloxypyridin-4-yl)boronic acid (4.2 g, 19.5 mmol, 1.1 equiv.), PdCl₂(dppf) (1.3 g, 1.8 mmol, 0.1 equiv.) and potassium phosphate (7.6 g, 36.0 mmol, 2 equiv.) in dioxane/water mixture (100 mL, 4:1 v/v) was degassed under vacuum and backfilled with nitrogen (repeated 3 times). The resulting suspension was stirred at 80° C. for 1.5 h. Once LCMS analysis confirmed complete consumption of the starting material the reaction mixture was allowed to cool to room temperature and partitioned between brine (50 mL) and EtOAc (150 mL). The organic phase was separated and the aqueous phase was additionally extracted with EtOAc (2×50 mL). The combined organic phase was dried over Na₂SO₄ and concentrated to dryness under reduced pressure. The crude product was purified by column chromatography (SiO₂, 0-30% EtOAc gradient in hexanes) to produce 4-(2-chloro-6-propan-2-yloxypyridin-4-yl)-3-(3,3-difluoroazetidine-1-carbonyl)-5-fluorobenzonitrile.

Steps g, h: The title compound was prepared in a similar fashion to that described for Example 96, steps e and f, to afford the title compound. ¹H NMR (400 MHz, CDCl₃) δ 12.26 (s, 1H), 8.54 (d, J=2.6 Hz, 1H), 7.98 (d, J=1.4 Hz, 1H), 7.67 (d, J=2.6 Hz, 1H), 7.59 (s, 1H), 7.54 (dd, J=8.8, 1.6 Hz, 1H), 6.51 (t, J=1.3 Hz, 1H), 5.45 (hept, J=6.2 Hz, 1H), 4.46-4.24 (m, 2H), 4.25-4.10 (m, 2H), 4.06-3.94 (m, 1H), 3.92-3.80 (m, 1H), 3.80-3.71 (m, 1H), 3.68 (s, 2H), 3.55-3.40 (m, 1H), 2.73 (dd, J=12.0, 3.5 Hz, 1H), 2.63 (dd, J=12.0, 7.8 Hz, 1H), 2.13-1.81 (m, 3H), 1.63-1.51 (m, 1H), 1.34 (d, J=6.2 Hz, 6H), 1.31-1.21 (m, 2H), 1.12-0.95 (m, 2H). ESI MS [M+H]⁺ for C₃₄H₃₅F₃N₆O₅, calcd 665.3, found 665.3

### Example 102: N-[4-[4-chloro-2-(3,3-difluoroazetidine-1-carbonyl)-6-fluorophenyl]-6-cyclopropylpyridin-2-yl]-1-cyclopropyl-5-[[[(2S)-2-methoxypropyl]amino]methyl]-2-oxopyridine-3-carboxamide

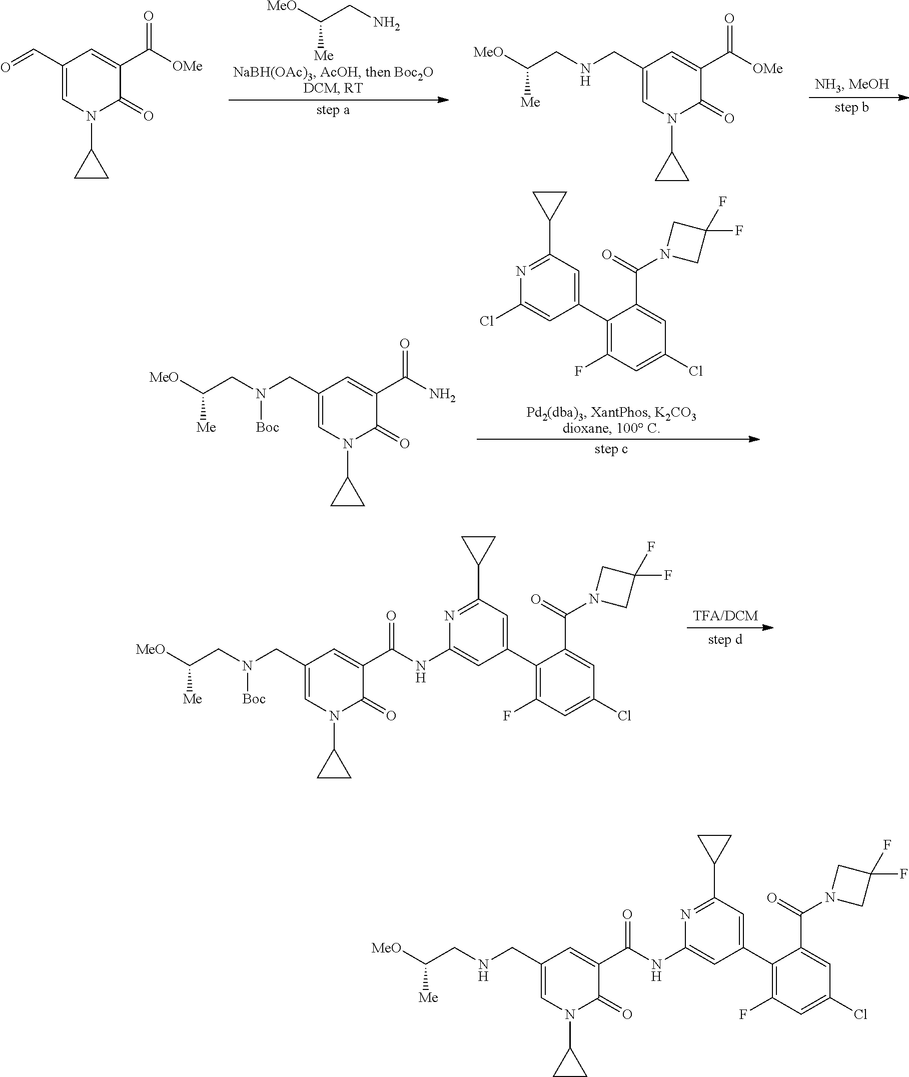

Step a: To a solution of methyl 1-cyclopropyl-5-formyl-2-oxopyridine-3-carboxylate (5.0 g, 22.6 mmol, 1.0 equiv.) in DCM (150 mL) was added (2S)-2-methoxypropan-1-amine (2.6 g, 29.4 mmol, 1.3 equiv.) followed by AcOH (1.9 mL, 33.9 mmol, 1.5 equiv.) and sodium triacetoxyborohydride (10.0 g, 45.2 mmol, 2.0 equiv.). The reaction mixture was stirred for 1.5 hours at room temperature at which point di-tert-butyl dicarbonate (7.4 g, 33.9 mmol, 1.5 equiv.) was added and the reaction was stirred for an additional 16 hours at room temperature. The reaction mixture was partitioned between H₂O (500 mL) and DCM (200 mL). The aqueous phase was extracted with DCM (2×100 mL) and the combined organics were dried over Na₂SO₄, filtered, and concentrated under vacuum. The crude residue was purified by column chromatography (SiO₂, 0-100% EtOAc in DCM) to afford the desired product.

Step b: A pressure vessel was charged with the product from step a (8.9 g, 22.6 mmol, 1.0 equiv.) and 7M NH₃ in MeOH (200 mL). The resulting solution was heated to 40° C. and stirred for 16 hours. The reaction mixture was cooled to room temperature and directly concentrated under vacuum. The crude residue was used without further purification.

Step c: A suspension of the product from step b (3.2 g, 8.5 mmol, 1.0 equiv.), [5-chloro-2-(2-chloro-6-cyclopropylpyridin-4-yl)-3-fluorophenyl]-(3,3-difluoroazetidin-1-yl)methanone (prepared in an analogous manner to that described in Example 84 using 2-bromo-5-chloro-3-fluorobenzoic acid in step a, 3.4 g, 8.5 mmol, 1.0 equiv.), and K₂CO₃ (3.5 g, 25.4 mmol, 3.0 equiv.) in dioxane (100 mL) was sparged with N₂ for 10 minutes. Pd₂(dba)₃ (192 mg, 0.21 mmol, 0.025 equiv.) and XantPhos (243 mg, 0.42 mmol, 0.05 equiv.) were added and the reaction mixture was heated to 100° C. and stirred for 2 hours. The reaction was cooled to room temperature and partitioned between EtOAc (200 mL) and a 2:1 mixture of water and saturated aqueous NaCl (250 mL). The aqueous phase was extracted with DCM (2×100 mL) and the combined organics were dried over Na₂SO₄, filtered, and concentrated under vacuum. The crude residue was purified by column chromatography (SiO₂, 0-100% EtOAc in hexanes) to afford the desired product.

Step d: To a solution of the product from step c (5.6 g, 5.6 mmol, 1.0 equiv.) in DCM (40 mL) was added TFA (10 mL). The reaction mixture was stirred at room temperature for 1 hour at which point it was diluted with PhMe (50 mL) and directly concentrated under vacuum. The crude residue was purified by column chromatography (C18, 5 to 60% MeCN in H₂O) to afford the desired product. ¹H NMR (400 MHz, Chloroform-d) δ 12.27 (s, 1H), 8.53 (d, J=2.6 Hz, 1H), 8.19 (s, 1H), 7.63 (d, J=2.6 Hz, 1H), 7.30-7.25 (m, 2H), 6.94 (s, 1H), 4.27 (t, J=11.8 Hz, 2H), 4.11 (t, J=11.5 Hz, 2H), 3.63 (d, J=2.5 Hz, 2H), 3.53-3.40 (m, 2H), 3.35 (s, 3H), 2.68-2.53 (m, 2H), 1.99 (tt, J=8.3, 4.7 Hz, 1H), 1.28-1.22 (m, 2H), 1.14 (d, J=6.1 Hz, 3H), 1.12-1.08 (m, 2H), 1.01-0.91 (m, 4H). ESI MS [M+H]⁺ for C₃₂H₃₃ClF₃N₅O₄ calcd 644.2 found 644.2.

### Example 103: N-(4-{4-Difluoromethoxy-2-[(3-fluoro-1-azetidinyl)carbonyl]phenyl}-6-(ethylamino)-2-pyridyl)-5-{[((S)-1-cyclobutylethylamino]methyl}-1-methyl-2-oxo-1,2-dihydronicotinamide

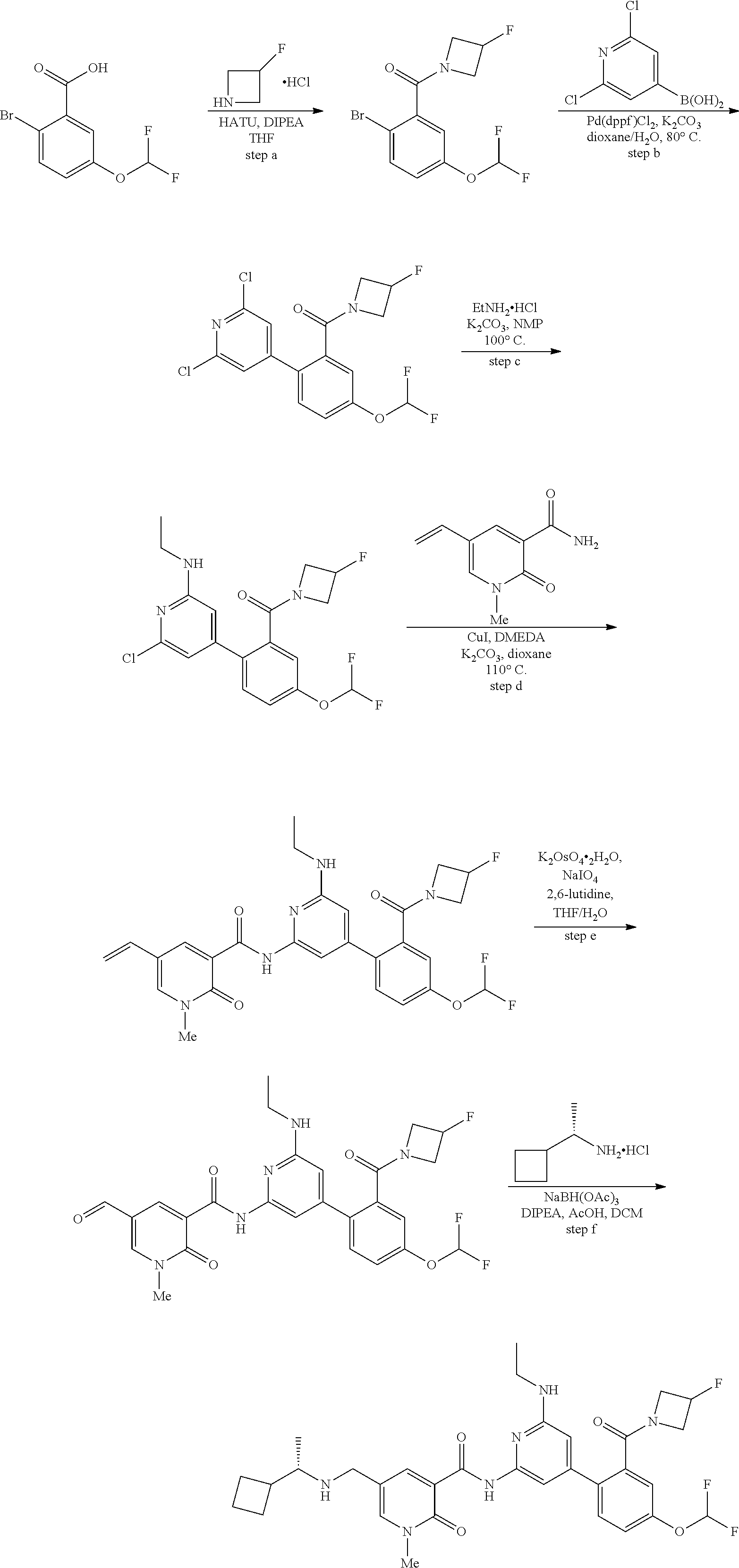

Step a: To a solution of 2-bromo-5-(difluoromethoxy)benzoic acid (2.67 g, 10 mmol) and 3-fluoroazetidine hydrochloride (1.34 g, 12 mmol) in THF (25 mL) was added HATU (4.56 g, 15 mmol) and DIPEA (2.6 mL, 1.94 g, 15 mmol) at room temperature. The resulting mixture was stirred at room temperature for overnight and then concentrated directly. The crude residue was purified by column chromatography (SiO₂, EtOAc in hexanes, 0 to 50%) to give the product.

Step b: To a solution of the product from step a (1.62 g, 5.0 mmol) and (2,6-dichloropyridin-4-yl)boronic acid (959 mg, 5.0 mmol) in dioxane (10 mL) was added Pd(dppf)Cl₂ (366 mg, 0.50 mmol) and 1M K₂CO₃ aqueous solution (10 mL, 10 mmol). The resulting mixture was then heated at 80° C. under N₂ for 1 h. After cooled to room temperature, the mixture was diluted with EtOAc and separated. The aqueous phase was extracted with EtOAc twice. The combined organic solution was then washed with brine, dried over Na₂SO₄, and concentrated. The crude residue was purified by column chromatography (SiO₂, EtOAc in hexanes, 0 to 50%) to give the product, [2-(2,6-dichloropyridin-4-yl)-5-(difluoromethoxy)phenyl]-(3-fluoroazetidin-1-yl)methanone.

Step c: To a solution of the product from step b (395 mg, 1.0 mmol) in NMP (4.0 mL) was added ethylamine hydrochloride (408 mg, 5.0 mmol) and K₂CO₃ (967 mg, 7.0 mmol). The resulting mixture was then heated at 100° C. for overnight. After cooled to room temperature, the mixture was diluted with EtOAc, washed with water three times, dried over Na₂SO₄, and concentrated. The crude residue was purified by column chromatography (SiO₂, EtOAc in hexanes, 0 to 50%) to give the product.

Step d: To a solution of the product from step c (157 mg, 0.39 mmol, 1.0 equiv.) and 5-ethenyl-1-methyl-2-oxopyridine-3-carboxamide (69.5 mg, 0.39 mmol, 1.0 equiv.) in dioxane (3.0 mL) was added CuI (150 mg, 0.78 mmol, 2.0 equiv.), DMEDA (0.17 mL, 141 mg, 1.6 mmol, 4.0 equiv.) and K₂CO₃ (166 mg, 1.2 mmol, 3.0 equiv.). The resulting mixture was heated at 110° C. under N₂ for overnight. After cooled to room temperature, the mixture was diluted with EtOAc and water, and then separated. The aqueous phase was extracted with EtOAc twice. The combined organic solution was washed with brine, dried over Na₂SO₄, and concentrated. The crude residue was purified by column chromatography (SiO₂, EtOAc in hexanes, 0 to 80%) to give the product.

Step e: To a solution of the product from step d (121 mg, 0.22 mmol, 1.0 equiv.) in THF/H₂O (4:1 v/v, 4.0 mL) was added K₂OsO₄·2H₂O (8.1 mg, 22 μmol, 10 mol %), NaIO₄ (385 mg, 1.8 mmol, 8.0 equiv.) and 2,6-lutidine (47.1 mg, 0.44 mmol, 2.0 equiv.). The resulting mixture was stirred at room temperature for 1 h and then quenched with saturated NaS₂O₃ and NaHCO₃ aqueous solution. The aqueous phase was extracted with EtOAc twice. The combined organic solution was then washed with brine, dried over Na₂SO₄, and concentrated. The crude residue was directly used in the next step.

Step f: To a solution of the crude product from step e (˜73 μmol) in DCM (1.0 mL) was added (S)-1-cyclobutylethanamine hydrochloride (20.3 mg, 0.15 mmol, 2.0 equiv.) and DIPEA (19.4 mg, 0.15 mmol, 2.0 equiv.). The resulting mixture was stirred at room temperature for 30 min before NaBH(OAc)₃ (31.8 mg, 0.15 mmol, 2.0 equiv.) and AcOH (9.0 mg, 0.15 mmol, 2.0 equiv.) was added. The reaction mixture was then stirred at room temperature for overnight. The mixture was then diluted with EtOAc, washed with water and bine, dried over Na₂SO₄, and concentrated. The crude residue was purified by HPLC to afford the title compound. ¹H NMR (400 MHz, DMSO-d₆) δ 12.29 (d, J=2.9 Hz, 1H), 8.51 (dd, J=6.1, 2.6 Hz, 1H), 8.06 (d, J=2.6 Hz, 1H), 7.61-7.15 (m, 5H), 6.69 (t, J=5.5 Hz, 1H), 6.21 (s, 1H), 5.64-5.35 (m, 1H), 4.21 (ddd, J=25.8, 20.9, 6.2 Hz, 2H), 3.98 (dt, J=23.7, 11.9 Hz, 2H), 3.78-3.58 (m, 4H), 3.55-3.46 (m, 1H), 3.30-3.19 (m, 2H), 2.47-2.38 (m, 1H), 2.27-2.09 (m, 1H), 2.09-1.92 (m, 1H), 1.92-1.82 (m, 2H), 1.76-1.51 (m, 4H), 1.16 (t, J=7.1 Hz, 3H), 0.94-0.77 (m, 3H). ESI MS [M+H]⁺ for C₃₂H₃₇F₃N₆O₄, calcd 627.3, found 627.3.

### Example 104: N-(4-{4-Difluoromethoxy-2-[(3-fluoro-1-azetidinyl)carbonyl]phenyl}-6-ethoxy-2-pyridyl)-5-{[(S)-2-methoxypropylamino]methyl}-1-cyclopropyl-2-oxo-1,2-dihydronicotinamide

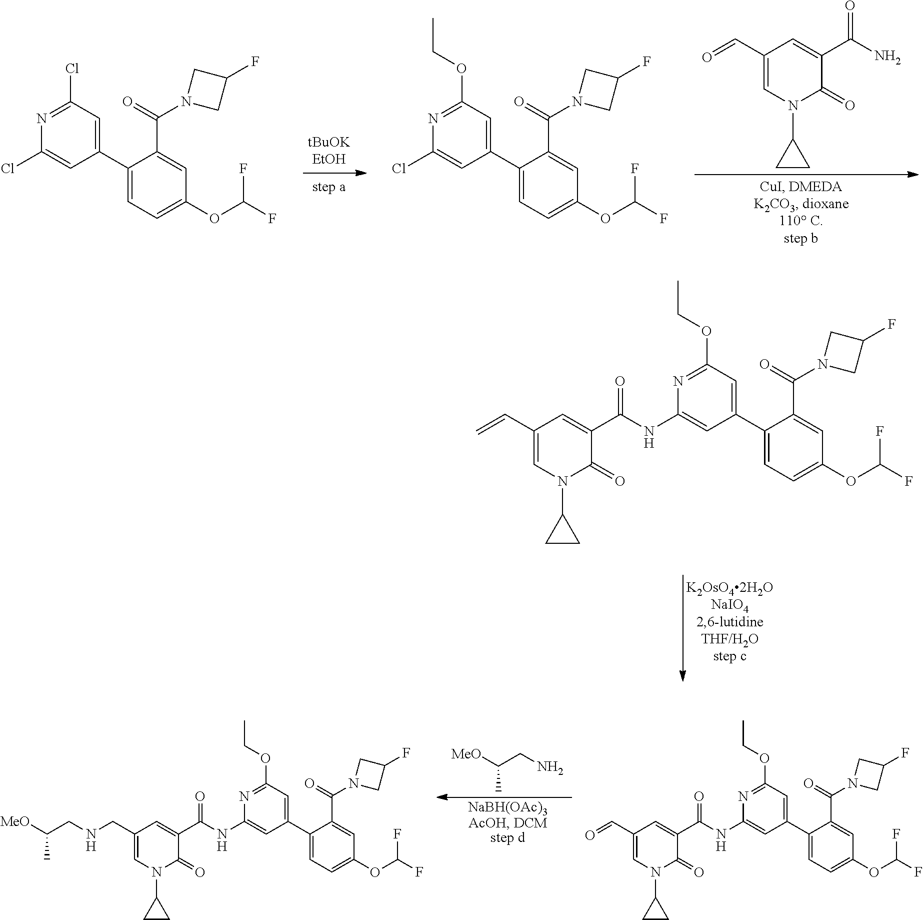

Step a: To a solution of [2-(2,6-dichloropyridin-4-yl)-5-(difluoromethoxy)phenyl]-(3-fluoroazetidin-1-yl)methanone (500 mg, 1.3 mmol, 1.0 equiv., prepared according to example 82 using 2-bromo-5-(difluoromethoxy)benzoic acid and 3-fluoroazetidine hydrochloride in step a) in ethanol (5.0 mL) was added t-BuOK (287 mg, 2.5 mmol, 2.0 equiv.) at room temperature. The resulting mixture was then heated at 80° C. for 2 h. After cooled to room temperature, the mixture was diluted with EtOAc and water, and then separated. The aqueous phase was extracted with EtOAc twice. The combined organic solution was then washed with brine, dried over Na₂SO₄, and concentrated. The crude residue was purified by column chromatography (SiO₂, EtOAc in hexanes, 0 to 50%) to give the product.

Step b: To a solution of the product from step a (227 mg, 0.57 mmol, 1.0 equiv.) and 1-cyclopropyl-5-ethenyl-2-oxopyridine-3-carboxamide (116 mg, 0.57 mmol, 1.0 equiv.) in dioxane (3.0 mL) was added CuI (217 mg, 1.1 mmol, 2.0 equiv.), DMEDA (0.25 mL, 203 mg, 2.3 mmol, 4.0 equiv.) and K₂CO₃ (235 mg, 1.7 mmol, 3.0 equiv.). The resulting mixture was heated at 110° C. under N₂ for overnight. After cooled to room temperature, the mixture was diluted with EtOAc and water, and then separated. The aqueous phase was extracted with EtOAc twice. The combined organic solution was then washed with brine, dried over Na₂SO₄, and concentrated. The crude residue was purified by column chromatography (SiO₂, EtOAc in hexanes, 0 to 80%) to give the product.

Step c: To a solution of the product from step b (179 mg, 0.31 mmol, 1.0 equiv.) in THF/H₂O (4:1 v/v, 4.0 mL) was added K₂OsO₄·2H₂O (11.6 mg, 31 μmol, 10 mol %), NaIO₄ (535 mg, 2.5 mmol, 8.0 equiv.) and 2,6-lutidine (66.4 mg, 0.62 mmol, 2.0 equiv.). The resulting mixture was stirred at room temperature for 1 h and then quenched with saturated NaS₂O₃ and NaHCO₃ aqueous solution. The aqueous phase was extracted with EtOAc twice. The combined organic solution was then washed with brine, dried over Na₂SO₄, and concentrated. The crude residue was directly used in the next step.

Step d: To a solution of the crude product from step c (˜0.10 mmol) in DCM (1.0 mL) was added (S)-2-methoxypropan-1-amine (17.8 mg, 0.20 mmol, 2.0 equiv.) and AcOH (12.1 mg, 0.20 mmol, 2.0 equiv.). The resulting mixture was stirred at room temperature for 30 min before NaBH(OAc)₃ (42.4 mg, 0.20 mmol, 2.0 equiv.) was added. The reaction mixture was then stirred at room temperature for overnight. The mixture was then diluted with EtOAc, washed with water and bine, dried over Na₂SO₄, and concentrated. The crude residue was purified by HPLC to afford the title compound. ¹H NMR (400 MHz, DMSO-d₆) δ 12.55 (s, 1H), 8.50 (d, J=2.5 Hz, 1H), 7.93 (d, J=2.6 Hz, 1H), 7.91 (d, J=1.2 Hz, 1H), 7.61 (d, J=8.5 Hz, 1H), 7.59-7.16 (m, 3H), 6.58 (d, J=1.2 Hz, 1H), 5.63-5.35 (m, 1H), 4.34 (q, J=7.1 Hz, 2H), 4.23 (ddq, J=17.6, 10.5, 6.0 Hz, 2H), 3.99 (td, J=22.0, 11.0 Hz, 2H), 3.58 (s, 2H), 3.51 (tt, J=7.6, 4.3 Hz, 1H), 3.44-3.35 (m, 1H), 3.23 (s, 3H), 2.57-2.40 (m, 2H), 1.36 (t, J=7.0 Hz, 3H), 1.16-1.08 (m, 2H), 1.06 (d, J=6.2 Hz, 3H), 1.03-0.93 (m, 2H). ESI MS [M+H]⁺ for C₃₂H₃₆F₃N₅O₆, calcd 644.3, found 644.3.

### Example 105: N-[4-[4-chloro-2-(3,3-difluoroazetidine-1-carbonyl)phenyl]-6-cyclopropylpyridin-2-yl]-5-[[[(2S)-2-methoxypropyl]amino]methyl]-1-methyl-2-oxopyridine-3-carboxamide

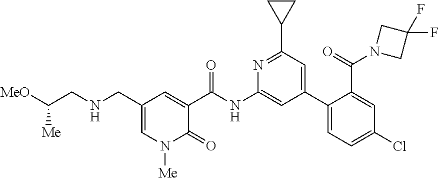 [SMILES: CO[C@@H](C)CNCc1cc(C(=O)Nc2cc(-c3ccc(Cl)cc3C(=O)N3CC(F)(F)C3)cc(C3CC3)n2)c(=O)n(C)c1]

The title compound was prepared in a similar fashion to that described for Example 90 using 5-ethenyl-1-methyl-2-oxopyridine-3-carboxamide in step c, and (2S)-2-methoxypropan-1-amine in step e. ¹H NMR (400 MHz, Chloroform-d) δ 12.32 (s, 1H), 8.56 (s, 1H), 8.24 (s, 1H), 7.65 (s, 1H), 7.53-7.46 (m, 3H), 6.97 (s, 1H), 4.33 (t, J=11.9 Hz, 2H), 3.96 (t, J=11.6 Hz, 2H), 3.70 (s, 3H), 3.67 (d, J=3.6 Hz, 2H), 3.54-3.43 (m, 1H), 3.35 (s, 3H), 2.70-2.56 (m, 2H), 2.00 (p, J=7.7, 7.1 Hz, 1H), 1.15 (d, J=6.1 Hz, 3H), 1.12-1.07 (m, 2H), 0.98-0.91 (m, 2H). ESI MS [M+H]⁺ for C₃₀H₃₂ClF₂N₅O₄ calcd 600.2 found 600.2.

### Example 106: N-[4-[4-chloro-2-(3,3-difluoroazetidine-1-carbonyl)phenyl]-6-cyclopropylpyridin-2-yl]-1-methyl-5-[(2-methylpropylamino)methyl]-2-oxopyridine-3-carboxamide

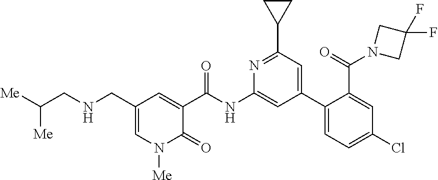 [SMILES: CC(C)CNCc1cc(C(=O)Nc2cc(-c3ccc(Cl)cc3C(=O)N3CC(F)(F)C3)cc(C3CC3)n2)c(=O)n(C)c1]

The title compound was prepared in a similar fashion to that described for Example 90 using 5-ethenyl-1-methyl-2-oxopyridine-3-carboxamide in step c, and isobutyl amine in step e. ¹H NMR (400 MHz, Chloroform-d) δ 12.33 (s, 1H), 8.56 (d, J=2.5 Hz, 1H), 8.24 (t, J=1.2 Hz, 1H), 7.65 (s, 1H), 7.53-7.43 (m, 3H), 6.97 (s, 1H), 4.33 (t, J=11.9 Hz, 2H), 3.97 (t, J=11.6 Hz, 2H), 3.70 (s, 3H), 3.66 (s, 2H), 2.44 (d, J=6.8 Hz, 2H), 2.00 (tt, J=8.5, 4.9 Hz, 1H), 1.84-1.70 (m, 1H), 1.17-1.04 (m, 2H), 0.98-0.93 (m, 2H), 0.93 (d, J=6.6 Hz, 6H). ESI MS [M+H]⁺ for C₃₀H₃₂ClF₂N₅O₃ calcd 584.2 found 584.3.

### Example 107: N-[4-[4-chloro-2-(3,3-difluoroazetidine-1-carbonyl)phenyl]-6-cyclopropylpyridin-2-yl]-1-methyl-2-oxo-5-[[[(2S)-oxolan-2-yl]methylamino]methyl]pyridine-3-carboxamide

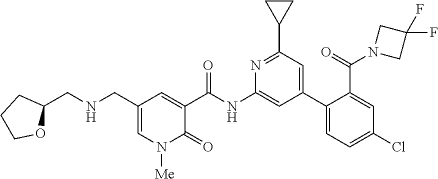 [SMILES: Cn1cc(CNC[C@@H]2CCCO2)cc(C(=O)Nc2cc(-c3ccc(Cl)cc3C(=O)N3CC(F)(F)C3)cc(C3CC3)n2)c1=O]

The title compound was prepared in a similar fashion to that described for Example 90 using 5-ethenyl-1-methyl-2-oxopyridine-3-carboxamide in step c. ¹H NMR (400 MHz, Chloroform-d) δ 12.33 (s, 1H), 8.56 (d, J=2.6 Hz, 1H), 8.24 (d, J=1.5 Hz, 1H), 7.66 (d, J=2.6 Hz, 1H), 7.51-7.47 (m, 3H), 6.97 (d, J=1.5 Hz, 1H), 4.33 (t, J=11.9 Hz, 2H), 4.06-3.91 (m, 3H), 3.85 (dt, J=8.3, 6.6 Hz, 1H), 3.75 (dt, J=8.3, 6.7 Hz, 1H), 3.70 (s, 5H), 2.74 (dd, J=12.0, 3.5 Hz, 1H), 2.64 (dd, J=12.0, 7.8 Hz, 1H), 2.03-1.80 (m, 4H), 1.14-1.04 (m, 2H), 1.01-0.90 (m, 2H). ESI MS [M+H]⁺ for C₃₁H₃₂ClF₂N₅O₄ calcd 612.2 found 612.3.

### Example 108: N-[4-[4-chloro-2-(3,3-difluoroazetidine-1-carbonyl)phenyl]-6-cyclopropylpyridin-2-yl]-1-methyl-5-[[[(2S)-oxan-2-yl]methylamino]methyl]-2-oxopyridine-3-carboxamide

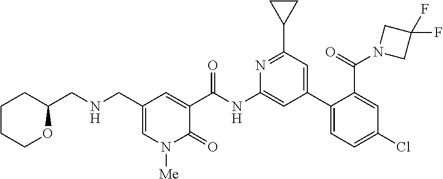 [SMILES: Cn1cc(CNC[C@@H]2CCCCO2)cc(C(=O)Nc2cc(-c3ccc(Cl)cc3C(=O)N3CC(F)(F)C3)cc(C3CC3)n2)c1=O]

The title compound was prepared in a similar fashion to that described for Example 90 using 5-ethenyl-1-methyl-2-oxopyridine-3-carboxamide in step c, and (2S)-tetrahydro-2H-pyran-2-methanamine in step e. ¹H NMR (400 MHz, Chloroform-d) δ 12.32 (s, 1H), 8.55 (d, J=2.5 Hz, 1H), 8.24 (s, 1H), 7.67 (d, J=2.5 Hz, 1H), 7.53-7.46 (m, 3H), 6.97 (s, 1H), 4.33 (t, J=11.9 Hz, 2H), 3.96 (t, J=11.8 Hz, 3H), 3.70 (s, 3H), 3.67 (d, J=4.5 Hz, 2H), 3.43 (td, J=11.0, 3.4 Hz, 2H), 2.69-2.59 (m, 2H), 2.00 (tt, J=8.5, 4.8 Hz, 1H), 1.84 (d, J=11.1 Hz, 1H), 1.59-1.45 (m, 4H), 1.40-1.24 (m, 1H), 1.12-1.06 (m, 2H), 0.98-0.89 (m, 2H). ESI MS [M+H]⁺ for C₃₂H₃₄ClF₂N₅O₄ calcd 626.2 found 626.3.

### Example 109: N-[6-cyclopropyl-4-[2-(3,3-difluoroazetidine-1-carbonyl)-4-(difluoromethoxy)phenyl]pyridin-2-yl]-1-methyl-5-[(2-methylpropylamino)methyl]-2-oxopyridine-3-carboxamide

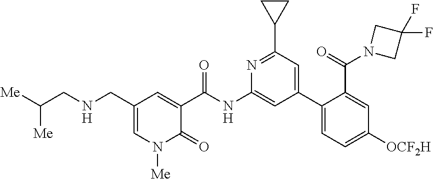

The title compound was prepared in a similar fashion to that described for Example 84 using 5-ethenyl-1-methyl-2-oxopyridine-3-carboxamide in step e and isobutyl amine in step g. ¹H NMR (400 MHz, Chloroform-d) δ 12.31 (s, 1H), 8.57 (d, J=2.6 Hz, 1H), 8.24 (d, J=1.5 Hz, 1H), 7.67 (s, 1H), 7.56 (d, J=9.2 Hz, 1H), 7.31-7.22 (m, 2H), 6.97 (d, J=1.5 Hz, 1H), 6.59 (t, J=73.1 Hz, 1H), 4.34 (t, J=11.9 Hz, 2H), 3.96 (t, J=11.6 Hz, 2H), 3.70 (s, 3H), 3.67 (s, 2H), 2.46 (d, J=6.8 Hz, 2H), 2.08-1.94 (m, 1H), 1.87-1.71 (m, 1H), 1.12-1.06 (m, 2H), 0.98-0.95 (m, 2H), 0.93 (d, J=6.7 Hz, 6H). ESI MS [M+H]⁺ for C₃₁H₃₃F₄N₅O₄ calcd 616.3 found 616.3.

### Example 110: N-(6-cyclopropyl-4-{2-[(3,3-difluoro-1-azetidinyl)carbonyl]-4-difluoromethoxyphenyl}-2-pyridyl)-5-[({[(R)-1,4-dioxan-2-yl]methyl}amino)methyl]-1-methyl-2-oxo-1,2-dihydronicotinamide

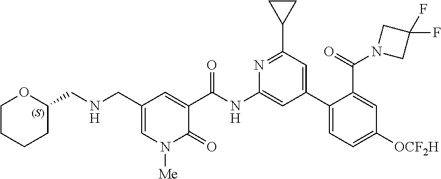

The title compound was prepared in a similar fashion to that described for Example 84 using 5-ethenyl-1-methyl-2-oxopyridine-3-carboxamide in step e and (2R)-1,4-dioxane-2-methanamine in step g. ¹H NMR (400 MHz, Chloroform-d) δ 12.30 (s, 1H), 8.56 (d, J=2.6 Hz, 1H), 8.24 (d, J=1.5 Hz, 1H), 7.67-7.52 (m, 2H), 7.32-7.26 (m, 2H), 6.97 (d, J=1.6 Hz, 1H), 6.80-6.37 (m, 1H), 4.34 (t, J=11.9 Hz, 2H), 3.96 (t, J=11.6 Hz, 2H), 3.77-3.69 (m, 7H), 3.66 (s, 2H), 3.63-3.55 (m, 1H), 3.43-3.34 (m, 1H), 2.68-2.57 (m, 2H), 2.06-1.96 (m, 1H), 1.35-1.23 (m, 1H), 1.13-1.06 (m, 2H), 0.99-0.92 (m, 2H). ESI MS [M+H]⁺ for C₃₃H₃₃F₄N₅O₆ calcd 660.2 found 660.2.

### Example 111: N-[6-cyclopropyl-4-[2-(3,3-difluoroazetidine-1-carbonyl)-4-(difluoromethoxy)phenyl]pyridin-2-yl]-1-methyl-5-[[[(2S)-oxan-2-yl]methylamino]methyl]-2-oxopyridine-3-carboxamide

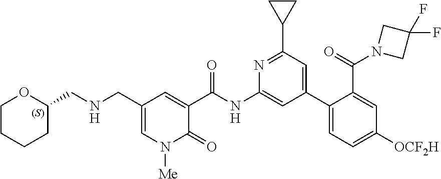

The title compound was prepared in a similar fashion to that described for Example 84 using 5-ethenyl-1-methyl-2-oxopyridine-3-carboxamide in step e and (2S)-tetrahydro-2H-pyran-2-methanamine in step g. ¹H NMR (400 MHz, Chloroform-d) δ 12.32 (s, 1H), 8.55 (d, J=2.6 Hz, 1H), 8.25 (d, J=1.5 Hz, 1H), 7.66 (d, J=2.6 Hz, 1H), 7.60-7.51 (m, 1H), 7.33-7.27 (m, 2H), 6.97 (d, J=1.5 Hz, 1H), 6.81-6.36 (m, 1H), 4.34 (t, J=11.9 Hz, 2H), 4.02-3.90 (m, 3H), 3.74-3.61 (m, 5H), 3.49-3.38 (m, 2H), 2.70-2.57 (m, 2H), 2.04-1.96 (m, 1H), 1.91-1.77 (m, 4H), 1.50-1.24 (m, 2H), 1.14-1.06 (m, 2H), 0.99-0.90 (m, 2H). ESI MS [M+H]⁺ for C₃₃H₃₅F₄N₅O₅ calcd 658.3 found 658.2.

### Example 112: N-[4-[4-cyano-2-(3,3-difluoroazetidine-1-carbonyl)phenyl]-6-propan-2-yloxypyridin-2-yl]-5-[[[(1S)-1-cyclobutylethyl]amino]methyl]-1-methyl-2-oxopyridine-3-carboxamide

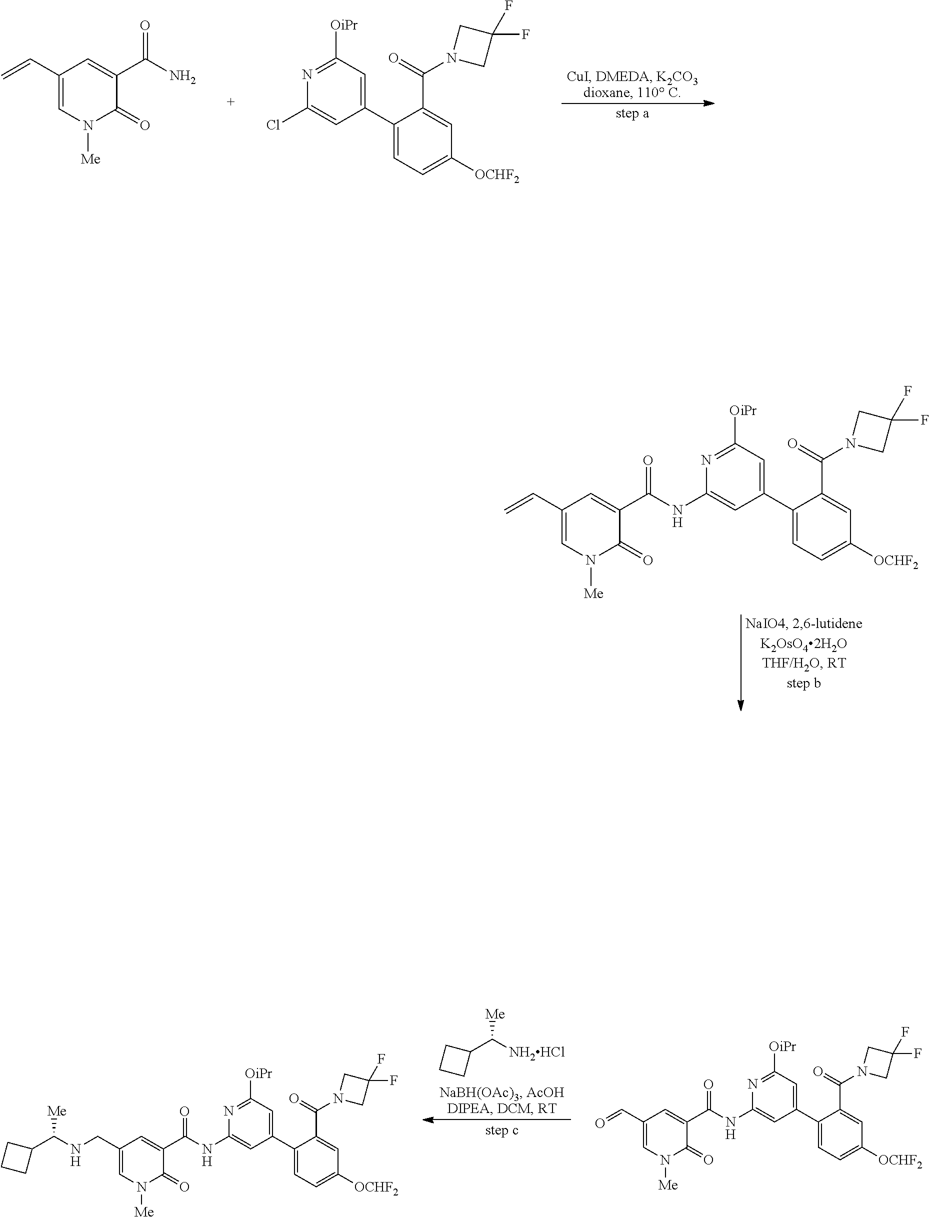

Step a: To a suspension of 5-ethenyl-1-methyl-2-oxopyridine-3-carboxamide (267 mg, 1.5 mmol, 1.0 equiv.) (prepared according to Example 94), [2-(2-chloro-6-propan-2-yloxypyridin-4-yl)-5-(difluoromethoxy)phenyl]-(3,3-difluoroazetidin-1-yl)methanone (650 mg, 1.5 mmol, 1.0 equiv., prepared according to Example 98), and K₂CO₃ (1040 mg, 7.5 mmol, 5.0 equiv.) in dioxane (5 mL) was added CuI (285 mg, 1.5 mmol, 1.0 equiv.) and DMEDA (528 mg, 4.3 mmol, 4.0 equiv.). The reaction mixture was heated to 110° C. and stirred for 90 hours, at which point it was quenched with saturated NH₄Cl solution (20 mL) and extracted with EtOAc (2×10 mL). The combined organics were dried over Na₂SO₄, filtered, and concentrated under a vacuum. The crude residue was purified by column chromatography (SiO₂, EtOAc in hexanes, 0 to 100%) to afford N-[4-[2-(3,3-difluoroazetidine-1-carbonyl)-4-(difluoromethoxy)phenyl]-6-propan-2-yloxypyridin-2-yl]-5-ethenyl-1-methyl-2-oxopyridine-3-carboxamide.

Step b: To a solution of the product from step a (332 mg, 0.62 mmol, 1.0 equiv.) in a mixture of THF (3 mL) and H₂O (3 mL) was added 2,6-lutidene (0.19 mL, 1.6 mmol, 2.0 equiv.) followed by NaIO₄ (686 mg, 3.2 mmol, 4.0 equiv.) and K₂OsO₄·2H₂O (15 mg, 0.04 mmol, 0.05 equiv.). The reaction mixture was stirred for 2 hours at room temperature at which point it was quenched with saturated aqueous Na₂S₂O₃ (10 mL) and extracted with EtOAc (2×10 mL). The combined organics were dried over Na₂SO₄, filtered, and concentrated under vacuum. The crude residue was used directly without further purification.

Step c: To a solution of the product from step b (60 mg, 0.1 mmol, 1.0 equiv.) in DCM (1.0 mL) was added (1S)-1-cyclobutylethanamine hydrochloride (27.0 mg, 0.2 mmol, 2.0 equiv.) followed by sodium triacetoxyborohydride (64 mg, 0.3 mmol, 3.0 equiv.), DIPEA (70 μL, 0.4 mmol, 2.0 equiv.) and AcOH (12 μL, 0.2 mmol, 2.0 equiv.). The reaction mixture was stirred for 16 hours at room temperature at which point it was quenched with H₂O (2 mL) and extracted with DCM (2×2 mL). The combined organics were dried over Na₂SO₄, filtered, and concentrated under a vacuum. The crude residue was purified by reverse phase prep-HPLC (C18 column, 5 to 60% MeCN/H₂O) to afford the desired product. ¹H NMR (400 MHz, Chloroform-d) δ 12.27 (s, 1H), 8.57 (d, J=2.6 Hz, 1H), 8.02 (d, J=1.3 Hz, 1H), 7.67 (d, J=2.6 Hz, 1H), 7.56 (d, J=9.3 Hz, 1H), 7.28 (d, J=2.8 Hz, 1H), 6.93-6.22 (m, 2H), 5.43 (p, J=6.2 Hz, 1H), 4.36 (t, J=12.0 Hz, 2H), 4.00 (t, J=11.6 Hz, 2H), 3.66 (d, J=35.8 Hz, 5H), 2.60 (dq, J=8.7, 6.3 Hz, 1H), 2.24 (q, J=8.3 Hz, 1H), 2.11-1.93 (m, 2H), 1.91-1.71 (m, 4H), 1.34 (d, J=6.2 Hz, 6H), 0.99 (d, J=6.2 Hz, 3H). ESI MS [M+H]⁺ for C₃₃H₃₈F₄N₅O₅, calcd 660.3, found 660.2.

### Example 113: N-(4-{2-[(3,3-difluoro-1-azetidinyl)carbonyl]-4-difluoromethoxyphenyl}-6-isopropoxy-2-pyridyl)-5-{[(S)-2-methoxypropylamino]methyl}-1-methyl-2-oxo-1,2-dihydronicotinamide

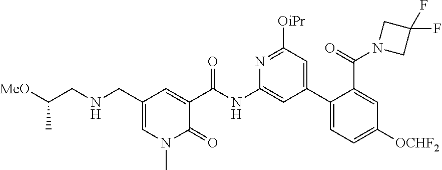 [SMILES: CO[C@@H](C)CNCc1cc(C(=O)Nc2cc(-c3ccc(OC(F)F)cc3C(=O)N3CC(F)(F)C3)cc(OC(C)C)n2)c(=O)n(C)c1]

The title compound was prepared in a similar fashion to that described for Example 112 using (2S)-2-methoxypropan-1-amine in step c. ¹H NMR (400 MHz, DMSO-d6) δ 12.43 (s, 1H), 8.61 (s, 1H), 7.88 (s, 1H), 7.70-7.15 (m, 4H), 6.55 (s, 1H), 5.76 (s, 1H), 5.26 (p, J=6.2 Hz, 1H), 4.33 (dt, J=15.7, 12.0 Hz, 4H), 3.91 (s, 2H), 3.66 (s, 3H), 3.53 (s, 1H), 3.28 (s, 3H), 2.73-2.56 (m, 3H), 1.34 (d, J=6.1 Hz, 6H), 1.11 (d, J=5.4 Hz, 3H). ESI MS [M+H]⁺ for C₃₁H₃₆F₄N₅O₆, calcd 650.3, found 650.2.

### Example 114: N-(4-{2-[(3,3-difluoro-1-azetidinyl)carbonyl]-4-difluoromethoxyphenyl}-6-isopropoxy-2-pyridyl)-5-{[((S)-1,2-dimethylpropylamino]methyl}-1-methyl-2-oxo-1,2-dihydronicotinamide

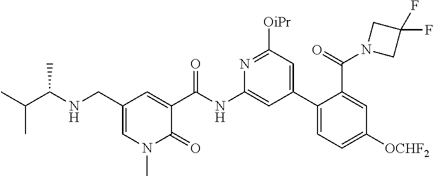 [SMILES: CC(C)Oc1cc(-c2ccc(OC(F)F)cc2C(=O)N2CC(F)(F)C2)cc(NC(=O)c2cc(CN[C@@H](C)C(C)C)cn(C)c2=O)n1]

The title compound was prepared in a similar fashion to that described for Example 112 using (2S)-3-methylbutan-2-amine in step c. ¹H NMR (400 MHz, Chloroform-d) δ 12.25 (s, 1H), 8.59 (d, J=2.6 Hz, 1H), 7.98 (d, J=1.3 Hz, 1H), 7.72 (d, J=2.5 Hz, 1H), 7.55 (d, J=9.3 Hz, 1H), 7.29 (d, J=2.6 Hz, 1H), 6.91-6.07 (m, 2H), 5.42 (p, J=6.1 Hz, 1H), 4.36 (t, J=12.0 Hz, 2H), 4.01 (t, J=11.6 Hz, 2H), 3.79 (d, J=13.5 Hz, 1H), 3.68 (d, J=8.2 Hz, 4H), 2.62 (q, J=6.1 Hz, 1H), 1.79 (ddd, J=13.7, 9.4, 5.9 Hz, 1H), 1.34 (d, J=6.2 Hz, 6H), 1.05 (d, J=6.5 Hz, 3H), 0.90 (t, J=6.6 Hz, 6H). ESI MS [M+H]⁺ for C₃₂H₃₈F₄N₅O₅, calcd 648.3, found 648.2.

### Example 115: N-(4-{2-[(3,3-difluoro-1-azetidinyl)carbonyl]-4-difluoromethoxyphenyl}-6-isopropoxy-2-pyridyl)-5-{[(S)-1-cyclopropylethylamino]methyl}-1-methyl-2-oxo-1,2-dihydronicotinamide

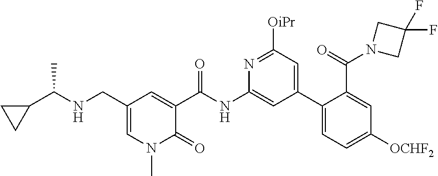 [SMILES: CC(C)Oc1cc(-c2ccc(OC(F)F)cc2C(=O)N2CC(F)(F)C2)cc(NC(=O)c2cc(CN[C@@H](C)C3CC3)cn(C)c2=O)n1]

The title compound was prepared in a similar fashion to that described for Example 112 using (1S)-1-cyclopropylethanamine in step c. ¹H NMR (400 MHz, Chloroform-d) δ 12.25 (s, 1H), 8.59 (d, J=2.6 Hz, 1H), 7.98 (d, J=1.3 Hz, 1H), 7.72 (d, J=2.5 Hz, 1H), 7.55 (d, J=9.3 Hz, 1H), 7.29 (d, J=2.6 Hz, 1H), 6.91-6.07 (m, 2H), 5.42 (p, J=6.1 Hz, 1H), 4.36 (t, J=12.0 Hz, 2H), 4.01 (t, J=11.6 Hz, 2H), 3.79 (d, J=13.5 Hz, 1H), 3.68 (d, J=8.2 Hz, 4H), 2.62 (q, J=6.1 Hz, 1H), 1.79 (ddd, J=13.7, 9.4, 5.9 Hz, 1H), 1.34 (d, J=6.2 Hz, 6H), 1.05 (d, J=6.5 Hz, 3H), 0.90 (t, J=6.6 Hz, 6H). ESI MS [M+H]⁺ for C₃₂H₃₆F₄N₅O₅, calcd 646.3, found 646.2.

### Example 116: N-(4-{2-[(3,3-difluoro-1-azetidinyl)carbonyl]-4-difluoromethoxyphenyl}-6-isopropoxy-2-pyridyl)-5-{[(1S,2S)-2-methoxy-1-methylpropylamino]methyl}-1-methyl-2-oxo-1,2-dihydronicotinamide

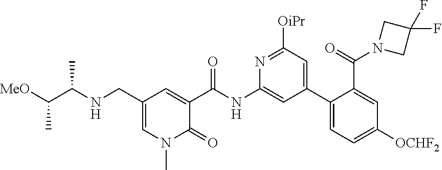 [SMILES: CO[C@@H](C)[C@H](C)NCc1cc(C(=O)Nc2cc(-c3ccc(OC(F)F)cc3C(=O)N3CC(F)(F)C3)cc(OC(C)C)n2)c(=O)n(C)c1]

The title compound was prepared in a similar fashion to that described for Example 112 using (2S,3S)-3-methoxybutan-2-amine in step c. ¹H NMR (400 MHz, Chloroform-d) δ 12.25 (s, 1H), 8.79-8.52 (m, 1H), 8.01 (s, 1H), 7.71-7.67 (m, 1H), 7.61-7.52 (m, 1H), 7.27 (s, 1H), 6.86-6.34 (m, 2H), 5.42 (p, J=6.2 Hz, 1H), 4.36 (t, J=12.0 Hz, 2H), 4.01 (t, J=11.7 Hz, 2H), 3.85-3.55 (m, 5H), 3.34 (s, 3H), 3.18 (s, 1H), 2.68 (d, J=8.0 Hz, 1H), 1.34 (d, J=6.1 Hz, 6H), 1.14 (d, J=5.8 Hz, 3H), 1.09 (d, J=6.5 Hz, 3H). ESI MS [M+H]⁺ for C₃₂H₃₈F₄N₅O₆, calcd 664.3, found 664.2.

### Example 117: N-(4-{4-cyano-2-[(3,3-difluoro-1-azetidinyl)carbonyl]phenyl}-6-isopropoxy-2-pyridyl)-5-{[(S)-2-methoxypropylamino]methyl}-1-methyl-2-oxo-1,2-dihydronicotinamide

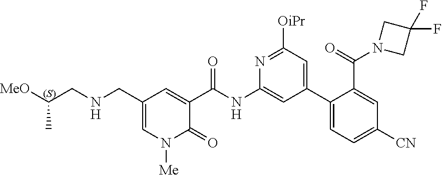

The title compound was prepared in a similar fashion to that described for Example 100 using (2S)-2-methoxypropan-1-amine in step c. ¹H NMR (400 MHz, Chloroform-d) δ 12.31 (s, 1H), 8.57 (d, J=2.5 Hz, 1H), 8.05 (d, J=1.3 Hz, 1H), 7.79 (d, J=6.9 Hz, 2H), 7.67 (d, J=6.5 Hz, 2H), 6.53 (d, J=1.4 Hz, 1H), 5.43 (p, J=6.2 Hz, 1H), 4.38 (t, J=12.0 Hz, 2H), 4.04 (t, J=11.5 Hz, 2H), 3.71 (d, J=1.4 Hz, 3H), 3.67 (d, J=3.5 Hz, 2H), 3.55-3.42 (m, 1H), 3.36 (d, J=1.4 Hz, 3H), 2.69-2.57 (m, 2H), 1.34 (dd, J=6.1, 1.4 Hz, 6H), 1.15 (dd, J=6.2, 1.4 Hz, 3H). ESI MS [M+H]⁺ for C₃₁H₃₄F₂N₆O₅ calcd. 609.3 found 609.3.

### Example 118: N-[4-[4-cyano-2-(3,3-difluoroazetidine-1-carbonyl)-6-fluorophenyl]-6-ethoxypyridin-2-yl]-1-methyl-2-oxo-5-[[[(2S)-oxolan-2-yl]methylamino]methyl]pyridine-3-carboxamide

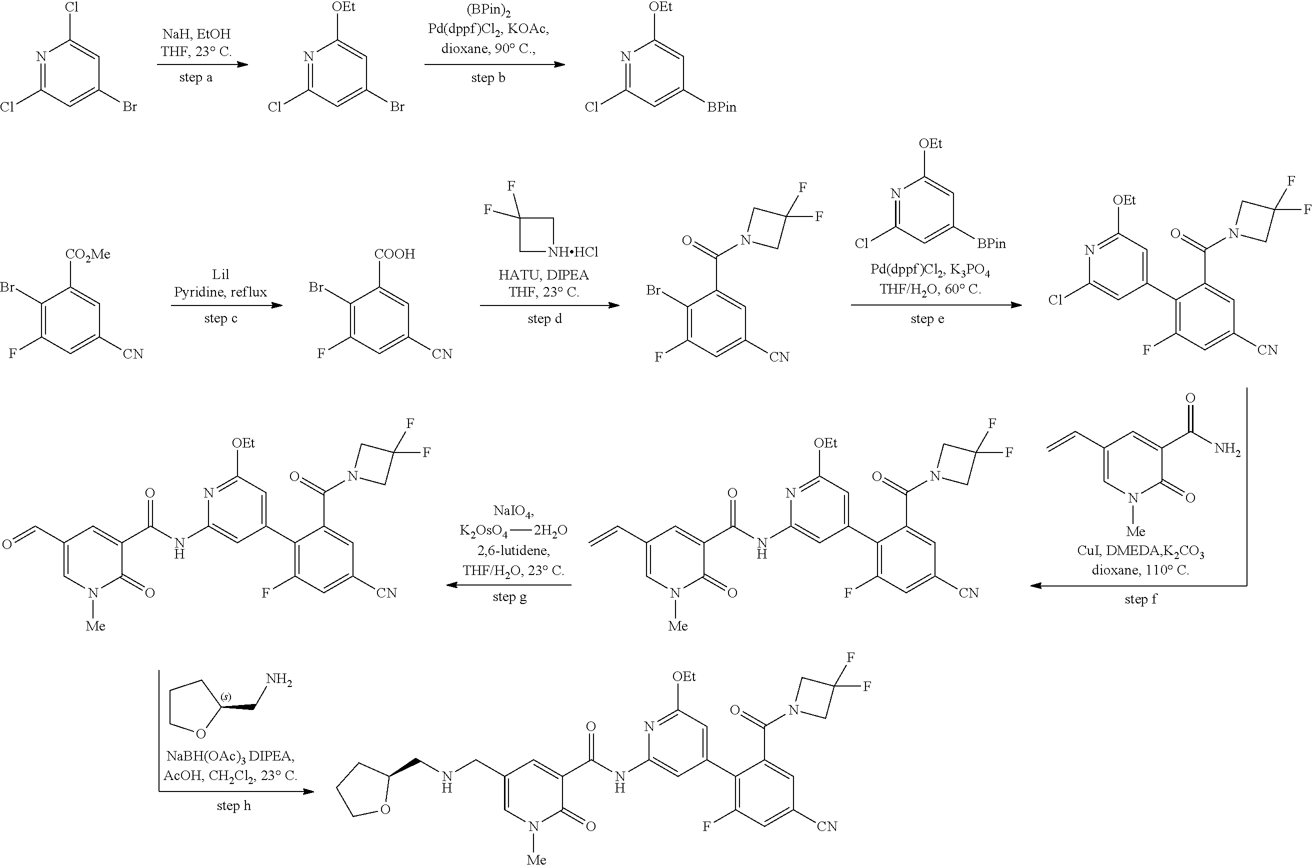

Step a: To a mixture of ethanol (12.8 mL, 0.22 mmol, 10.0 equiv.) in THF (110 mL, 0.2 M) was added NaH (0.97 g, 60% in mineral oil, 24.2 mmol, 1.10 equiv.) at 0° C. The cooling bath was removed, the mixture was stirred at the room temperature for 1 h, and 4-bromo-2,6-dichloropyridine (5.0 g, 22.0 mmol, 1.0 equiv.) was added to the reaction mixture. It was stirred for 4 h at room temperature. Then, the reaction mixture was quenched with water (40 mL) and extracted with EtOAc (2×80 mL). The organic phase was dried over Na₂SO₄, concentrated and the crude residue was purified by column chromatography (SiO₂, EtOAc in hexanes, 0 to 10%) to give the desired product.

Step b: A 100 mL round bottom flask was charged with product from step a (1.0 g, 4.22 mmol, 1.0 equiv.), B₂Pin₂ (1.16 g, 4.64 mmol, 1.10 equiv.), KOAc (1.44 g, 14.8 mmol, 3.50 equiv.) and Pd(dppf)Cl₂ (0.31 g, 0.42 mmol, 0.10 equiv.). The reagents were suspended in the dioxane (21 mL, 0.20 M). Then, the reaction mixture was sparged with N₂ for 10 minutes and heated to 80° C. for 1 hour. The resulting solution was cooled to ambient temperature and diluted with brine (10 mL) and EtOAc (40 mL). The organic phase was separated, and the aqueous phase was additionally extracted with EtOAc (20 mL). The combined organics were dried over Na₂SO₄, filtered, and concentrated to dryness under reduced pressure to give the desired product.

Step c: A mixture of methyl 2-bromo-5-cyano-3-fluorobenzoate (6.0 g, 23.3 mmol, 1.0 equiv.) and LiI (15.6 g, 0.12 mol, 5.0 equiv.) in pyridine (230 ml, 0.10 M) was heated to reflux for 3 h. The reaction mixture was cooled to room temperature and concentrated to ˜40 mL. The residue was acidified to pH-1 with aq. HCl (4 M), and the formed product was extracted with EtOAc (2×40 mL). The combined organics were dried over Na₂SO₄, filtered, and concentrated under reduced pressure. The crude residue was used in the next step without further purification.

Step d: To a solution of crude material from step c (5.70 g, 23.3 mmol, 1.0 equiv.) in THF (120 mL, 0.20 M) were added DIPEA (12.0 mL, 70.0 mmol, 3.0 equiv.), HATU (11.5 g, 30.3 mmol, 1.30 equiv.) and 3,3-difluoroazetidine hydrochloride (3.90 g, 30.3 mmol, 1.30 equiv.). The reaction mixture was stirred overnight at room temperature and diluted with EtOAc (200 mL) and water (100 mL). The organic phase was separated, washed with brine (100 mL), dried over Na₂SO₄ and concentrated to dryness under reduced pressure. The crude residue was purified by column chromatography (SiO₂, EtOAc in hexanes, 0 to 80%) to afford the desired product.

Step e: A suspension of the product from step d (1.48 g, 4.64 mmol, 1.10 equiv.), the product from step b (1.20 g, 4.22 mmol, 1.0 equiv.) and K₃PO₄ (2.0 g, 9.28 mmol, 2.0 equiv.) in THF/water mixture (24 mL, 4:1 v/v, 0.2 M) was purged with N₂ for 10 minutes. Pd(dppf)Cl₂ (0.15 g, 0.21 mmol, 0.05 equiv.) was added, and the reaction was heated to 60° C. for 1 h. Then, it was cooled to room temperature, diluted with a ˜1:1 mixture of saturated aqueous NaCl/water (20 mL) and extracted with EtOAc (2×30 mL). The combined organics were dried over Na₂SO₄, filtered, and concentrated under vacuum. The crude residue was purified via column chromatography (SiO₂, EtOAc in hexanes, 0 to 40%), followed by purification via reversed-phase column chromatography (C18, 10-100% CH₃CN in water with 0.1% formic acid) to afford the desired product.

Step f: A solution of 5-ethenyl-1-methyl-2-oxopyridine-3-carboxamide (0.23 g, 1.26 mmol, 1.0 equiv., prepared according to the protocol described for Example 87) and the product from step e (0.50 g, 1.26 mmol, 1.0 equiv.) and K₂CO₃ (1.0 g, 7.56 mmol, 6.0 equiv.) in dioxane (25 mL, 0.05M) was degassed with a stream of bubbling nitrogen for ten minutes. CuI (0.24 g, 1.26 mmol, 1.0 equiv.) and DMEDA (0.12 mL, 1.26 mmol, 1.0 equiv.) were added, and the reaction was heated for 16 h at 100° C. Then, the mixture was cooled to room temperature and diluted with aq. NH₄Cl (20 mL) and extracted with EtOAc (2×50 mL). The combined organic phase was washed with water (2×20 mL) and brine (20 mL), dried over Na₂SO₄, and concentrated to dryness. The crude residue was purified by reversed-phase column chromatography (C18, 10-100% CH₃CN in water with 0.1% formic acid) to afford the desired product.

Step g: To a solution of the product from step f (0.36 g, 0.67 mmol, 1.0 equiv.) in THF/H₂O (1:1 v/v, 6.80 mL) was added K₂OsO₄·2H₂O (25 mg, 67 μmol, 0.10 equiv.), NaIO₄ (0.57 g, 2.68 mmol, 4.0 equiv.) and 2,6-lutidine (0.16 mL, 1.34 mmol, 2.0 equiv.). The resulting mixture was stirred at room temperature for 1 h and then quenched with saturated NaS₂O₃ (6 mL) and NaHCO₃ (6 mL) aqueous solution. The aqueous phase was extracted with EtOAc (2×10 mL). The combined organic solution was then washed with brine (10 mL), dried over Na₂SO₄, and concentrated under reduced pressure. The crude residue was directly used in the next step.

Step h: To a solution of the product from step g (33 mg, 0.061 mmol, 1.0 equiv.) in DCM (1.20 mL) was added [(2S)-oxolan-2-yl]methanamine (12 mg, 0.12 mmol, 2.0 equiv.) followed by AcOH (7 μL, 0.12 mmol, 2.0 equiv.) and DIPEA (21 μL, 0.12 mmol, 2.0 equiv.). The resulting mixture was stirred at room temperature for 30 min before NaBH(OAc)₃ (26 mg, 0.12 mmol, 2.0 equiv.) was added. The reaction mixture was stirred for 16 hours at room temperature at which point it was quenched with MeOH (1.0 mL), and evaporated to dryness. The crude residue was purified by reverse phase prep-HPLC (C18 column, 5 to 60% MeCN/H₂O) to afford the title compound. ¹H NMR (400 MHz, Chloroform-d) δ 12.35 (s, 1H), 8.54 (d, J=2.5 Hz, 1H), 7.98 (s, 1H), 7.67 (d, J=2.6 Hz, 1H), 7.58 (d, J=1.6 Hz, 1H), 7.54 (dd, J=8.7, 1.6 Hz, 1H), 6.54 (d, J=1.4 Hz, 1H), 4.40 (q, J=7.1 Hz, 2H), 4.32 (t, J=11.7 Hz, 2H), 4.16 (q, J=11.2 Hz, 2H), 4.00 (qd, J=7.4, 3.4 Hz, 1H), 3.89-3.79 (m, 1H), 3.74 (q, J=7.3 Hz, 2H), 3.69 (d, J=3.6 Hz, 3H), 2.72 (dd, J=11.9, 3.5 Hz, 1H), 2.63 (dd, J=11.9, 7.8 Hz, 1H), 1.92 (ddt, J=31.7, 13.5, 6.6 Hz, 3H), 1.75 (s, 2H), 1.55 (dq, J=11.2, 7.6 Hz, 1H), 1.38 (t, J=7.1 Hz, 3H). ESI MS [M+H]⁺ for C₃₁H₃₁F₃N₆O₅ calcd 625.2 found 625.2.

### Example 119: N-[4-[4-cyano-2-(3,3-difluoroazetidine-1-carbonyl)-6-fluorophenyl]-6-ethoxypyridin-2-yl]-5-[[[(1S)-1-cyclopropylethyl]amino]methyl]-1-methyl-2-oxopyridine-3-carboxamide

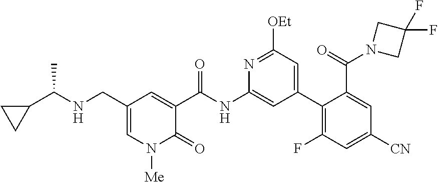 [SMILES: CCOc1cc(-c2c(F)cc(C#N)cc2C(=O)N2CC(F)(F)C2)cc(NC(=O)c2cc(CN[C@@H](C)C3CC3)cn(C)c2=O)n1]

The title compound was prepared in a similar fashion to that described for example 118 using (1S)-1-cyclopropylethanamine hydrochloride in step h. ¹H NMR (400 MHz, Chloroform-d) δ 12.34 (s, 1H), 8.54 (d, J=2.6 Hz, 1H), 7.97 (s, 1H), 7.68 (d, J=2.6 Hz, 1H), 7.58 (d, J=1.4 Hz, 1H), 7.54 (dd, J=8.8, 1.5 Hz, 1H), 6.54 (d, J=1.5 Hz, 1H), 4.40 (q, J=7.1 Hz, 2H), 4.31 (t, J=11.8 Hz, 2H), 4.17 (t, J=11.3 Hz, 2H), 3.71 (d, J=16.6 Hz, 5H), 1.95 (dp, J=12.7, 6.4 Hz, 1H), 1.37 (t, J=7.1 Hz, 3H), 1.17 (d, J=6.3 Hz, 3H), 0.73 (qt, J=8.5, 5.0 Hz, 1H), 0.48 (dtt, J=26.5, 8.9, 4.4 Hz, 2H), 0.19 (dq, J=9.3, 4.8 Hz, 1H), 0.12-0.07 (m, 1H). ESI MS [M+H]⁺ for C₃₁H₃₁F₃N₆O₄ calcd 609.2 found 609.2.

### Example 120: N-[4-[4-cyano-2-(3,3-difluoroazetidine-1-carbonyl)-6-fluorophenyl]-6-ethoxypyridin-2-yl]-5-[[[(2S)-2-methoxypropyl]amino]methyl]-1-methyl-2-oxopyridine-3-carboxamide

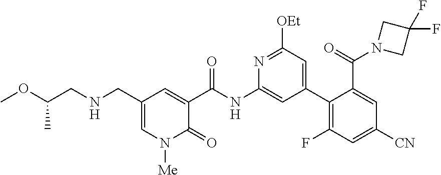 [SMILES: CCOc1cc(-c2c(F)cc(C#N)cc2C(=O)N2CC(F)(F)C2)cc(NC(=O)c2cc(CNC[C@H](C)OC)cn(C)c2=O)n1]

The title compound was prepared in a similar fashion to that described for example 118 using (2S)-2-methoxypropan-1-amine in step h. ¹H NMR (400 MHz, Chloroform-d) δ 12.32 (s, 1H), 8.56 (d, J=2.6 Hz, 1H), 7.97 (s, 1H), 7.64 (d, J=2.6 Hz, 1H), 7.58 (d, J=1.6 Hz, 1H), 7.54 (dd, J=8.7, 1.6 Hz, 1H), 7.31-7.23 (m, 1H), 6.54 (d, J=1.4 Hz, 1H), 4.40 (q, J=7.1 Hz, 2H), 4.32 (t, J=11.7 Hz, 2H), 4.16 (q, J=11.2 Hz, 2H), 3.70 (s, 3H), 3.66 (d, J=4.3 Hz, 2H), 3.53-3.44 (m, 1H), 3.35 (s, 3H), 2.68-2.57 (m, 2H), 1.37 (t, J=7.1 Hz, 3H), 1.15 (d, J=6.2 Hz, 3H). ESI MS [M+H]⁺ for C₃₀H₃₁F₃N₆O₅ calcd 613.2 found 613.2.

### Example 121: N-(4-{4-chloro-2-[(3,3-difluoro-1-azetidinyl)carbonyl]phenyl}-6-ethoxy-2-pyridyl)-1-cyclopropyl-5-({[(1-fluorocyclobutyl)methyl]amino}methyl)-2-oxo-1,2-dihydronicotinamide

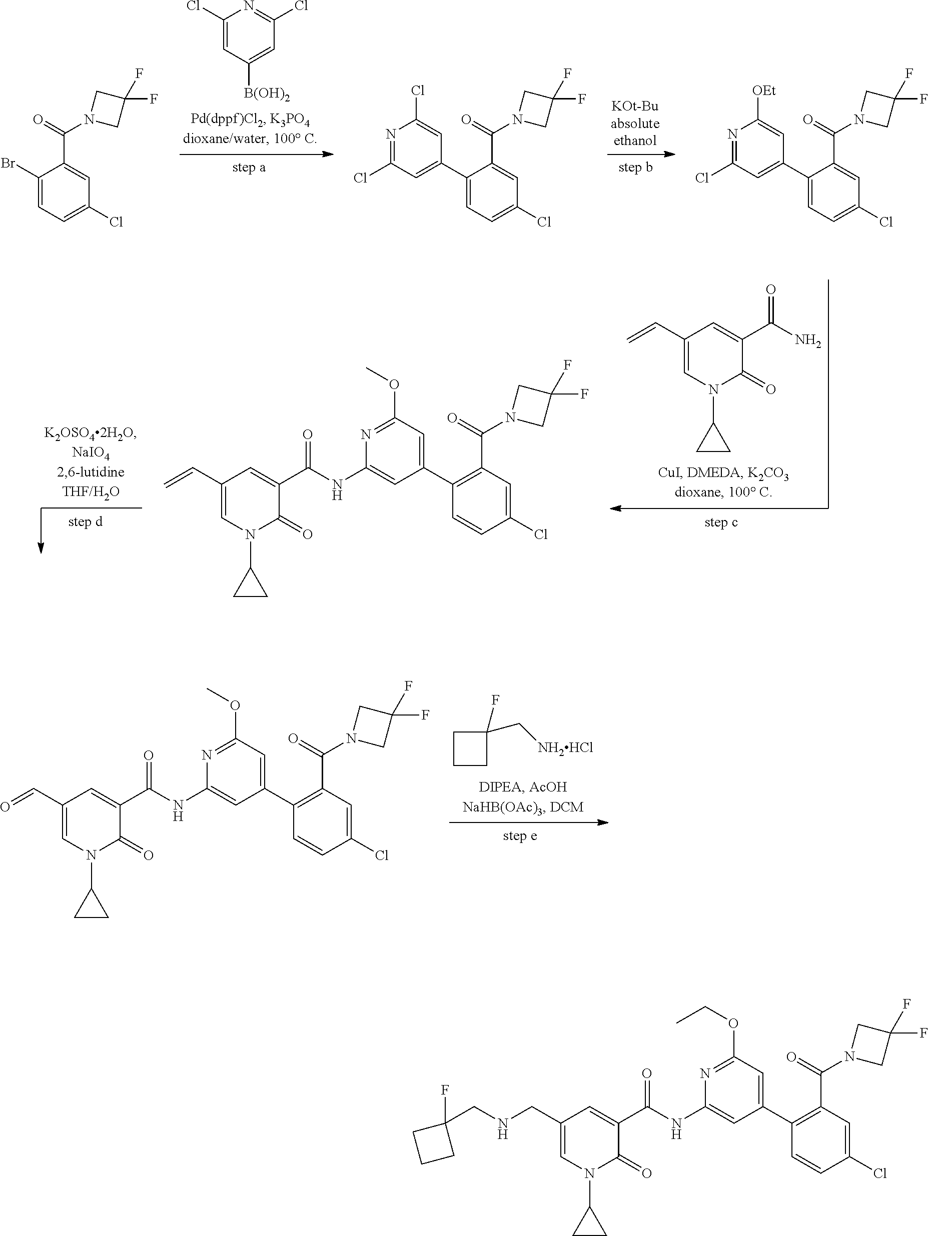

Step a: A 250 mL round bottom flask was charged with (2-bromo-5-chlorophenyl)-(3,3-difluoroazetidin-1-yl)methanone (3.2 g, 10.1 mmol, 1.0 equiv.), (2,6-dichloropyridin-4-yl)boronic acid (5.1 g, 26.6 mmol. 2.6 equiv.) and potassium phosphate (7.0 g, 32.97 mmol, 3.2 equiv.). The reagents were suspended in the 4:1 mixture of dioxane/water (81 mL). Then Pd(dppf)Cl₂ (1.2 g, 1.64 mmol, 0.16 equiv.) was added, and the mixture was heated to 100° C. for 6 hours. The reaction mixture was partitioned between EtOAc and water. The organic phase was separated, and the aqueous phase was additionally extracted with EtOAc (2×50 mL). The combined organics were dried over Na₂SO₄ and concentrated to dryness. The crude residue was purified via silica gel flash column chromatography (EtOAc in hexanes, 0 to 100%) to afford [5-chloro-2-(2,6-dichloropyridin-4-yl)phenyl]-(3,3-difluoroazetidin-1-yl)methanone.

Step b: A solution of KOtBu (0.89 g, 7.94 mmol, 3.0 equiv.) in absolute ethanol (13 mL) was stirred for 5 minutes at room temperature. Then the product from step a (1.0 g, 2.65 mmol, 1.0 equiv.) was added and heated at 90° C. for 30 minutes, at which point it was quenched with a ˜1:1 mixture of saturated aqueous NaCl/water (10 mL) and extracted with EtOAc (2×50 mL). The combined organics were dried over Na₂SO₄, filtered, and concentrated under a vacuum. The crude residue was purified via silica gel flash column chromatography (EtOAc in hexanes, 0 to 100%) to afford the desired product.

Step c: To a mixture of the product from step b (947 mg, 2.44 mmol, 1.0 equiv.) and 1-cyclopropyl-5-ethenyl-2-oxopyridine-3-carboxamide (500 mg, 2.44 mmol, 1.0 equiv.) in dioxane (49 mL, 0.05 M) was added CuI (511 mg, 2.68 mmol, 1.0 equiv.), DMEDA (430 mg, 88.2 mmol, 1.1 equiv.) and K₂CO₃ (2.0 g, 14.64 mmol, 6.0 equiv.). The resulting mixture was heated at 100° C. under N₂ for 40 h under vigorous stirring. The reaction was allowed to cool to rt, diluted with water (20 mL) and EtOAc (50 mL), the organic phase was separated, and the aqueous layer was additionally extracted with EtOAc (2×25 mL). The combined organic phase was dried over MgSO₄, concentrated and the crude residue was purified by column chromatography (EtOAc in hexanes, 0 to 100%) to afford the desired product.

Step d: To a solution of the product from step c (0.27 g, 0.47 mmol, 1.0 equiv.) in a 1:1 mixture of THF/H₂O (5 mL) was added 2,6-lutidine (0.1 mL, 0.93 mmol, 2.0 equiv.) followed by NaIO₄ (995 mg, 4.7 mmol, 10.0 equiv.) and K₂OsO₄*2H₂O (17 mg, 0.05 mmol, 0.1 equiv.). The reaction mixture was stirred for 4 hours at room temperature, at which point it was quenched with saturated aqueous Na₂S₂O₃ (10 mL) and extracted with EtOAc (2×50 mL). The combined organics were dried over Na₂SO₄, filtered, and concentrated under a vacuum. The crude residue was used directly without further purification.

Step e: To a solution of the crude product from step d (˜0.07 mmol, 1.0 equiv.) in DCM (1.0 mL) was added (1-fluorocyclobutyl)methanamine hydrochloride (20 mg, 0.14 mmol, 2.0 equiv.) followed by sodium triacetoxyborohydride (30 mg, 0.14 mmol, 2.0 equiv.) and AcOH (13 μL, 0.14 mmol, 2.0 equiv.). The reaction mixture was stirred for 16 hours at room temperature. The mixture was then diluted with EtOAc, washed with water and brine, dried over Na₂SO₄, and concentrated. The crude residue was purified by HPLC to afford the title compound. ¹H NMR (400 MHz, CDCl₃) δ 12.25 (s, 1H), 8.56 (d, J=2.6 Hz, 1H), 8.03 (d, J=1.1 Hz, 1H), 7.71 (d, J=2.6 Hz, 1H), 7.49 (d, J=4.5 Hz, 3H), 6.55 (d, J=1.1 Hz, 1H), 4.45-4.30 (m, 3H), 4.01 (t, J=11.6 Hz, 2H), 3.70 (s, 2H), 3.46 (tt, J=7.7, 4.2 Hz, 1H), 2.48 (d, J=6.8 Hz, 3H), 1.79 (hept, J=6.7 Hz, 1H), 1.42-1.34 (m, 3H), 1.24 (p, J=8.2, 7.6 Hz, 2H), 0.98 (dd, J=6.7, 4.4 Hz, 2H), 0.95-0.86 (m, 6H). ESI MS [M+H]⁺ C₃₂H₃₃ClF₃N₅O₄, calcd 644.2, found 644.2.

### Example 122: N-(4-{4-chloro-2-[(3,3-difluoro-1-azetidinyl)carbonyl]phenyl}-6-ethoxy-2-pyridyl)-5-{[(S)-1-cyclobutylethylamino]methyl}-1-cyclopropyl-2-oxo-1,2-dihydronicotinamide

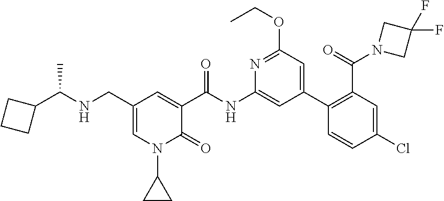 [SMILES: CCOc1cc(-c2ccc(Cl)cc2C(=O)N2CC(F)(F)C2)cc(NC(=O)c2cc(CN[C@@H](C)C3CCC3)cn(C3CC3)c2=O)n1]

The title compound was prepared in a similar fashion to that described in Example 121 using (1S)-1-cyclobutylethan-1-amine hydrochloride in step e. ¹H NMR (400 MHz, CDCl₃) δ 12.23 (s, 1H), 8.54 (d, J=2.6 Hz, 1H), 8.02 (d, J=1.3 Hz, 1H), 7.72 (d, J=2.6 Hz, 1H), 7.51-7.45 (m, 3H), 6.55 (d, J=1.3 Hz, 1H), 4.37 (dd, J=13.3, 6.4 Hz, 4H), 4.00 (t, J=11.6 Hz, 2H), 3.73 (d, J=13.4 Hz, 1H), 3.62 (d, J=13.4 Hz, 1H), 3.44 (tt, J=7.6, 4.3 Hz, 1H), 2.81 (s, 1H), 2.68-2.59 (m, 1H), 2.29 (p, J=8.2 Hz, 1H), 2.01-1.93 (m, 1H), 1.71 (tt, J=16.4, 8.2 Hz, 3H), 1.37 (t, J=7.1 Hz, 3H), 1.22 (t, J=6.7 Hz, 2H), 0.98 (dd, J=15.8, 5.5 Hz, 5H). ESI MS [M+H]⁺ for C₃₃H₃₇ClF₂N₅O₄, calcd 640.2, found 640.3.

### Example 123: N-(4-{4-chloro-2-[(3,3-difluoro-1-azetidinyl)carbonyl]phenyl}-6-ethoxy-2-pyridyl)-1-cyclopropyl-5-{[(S)-2-methoxypropylamino]methyl}-2-oxo-1,2-dihydronicotinamide

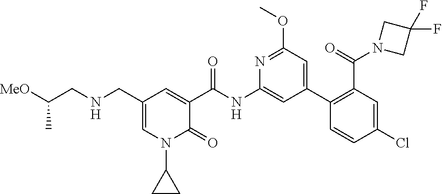 [SMILES: COc1cc(-c2ccc(Cl)cc2C(=O)N2CC(F)(F)C2)cc(NC(=O)c2cc(CNC[C@H](C)OC)cn(C3CC3)c2=O)n1]

The title compound was prepared in a similar fashion to that described in Example 121 using (2S)-2-methoxypropan-1-amine in step e. ¹H NMR (400 MHz, CDCl₃) δ 12.25 (s, 1H), 8.55 (d, J=2.6 Hz, 1H), 8.03 (d, J=1.2 Hz, 1H), 7.67 (d, J=2.6 Hz, 1H), 7.48 (d, J=4.2 Hz, 3H), 6.55 (d, J=1.2 Hz, 1H), 4.45-4.30 (m, 4H), 4.00 (t, J=11.6 Hz, 2H), 3.67 (s, 2H), 3.47 (dtt, J=15.8, 7.5, 4.1 Hz, 2H), 3.34 (s, 3H), 2.71-2.56 (m, 2H), 2.17 (d, J=2.5 Hz, 2H), 1.37 (t, J=7.1 Hz, 3H), 1.24 (t, J=6.8 Hz, 2H), 1.14 (d, J=6.2 Hz, 3H), 1.01-0.92 (m, 2H). ESI MS [M+H]⁺ for C₃₂H₃₅ClF₂N₅O₄, calcd 630.2, found 630.2.

### Example 124: N-(4-{4-chloro-2-[(3,3-difluoro-1-azetidinyl)carbonyl]phenyl}-6-ethoxy-2-pyridyl)-1-cyclopropyl-5-{[((S)-1-cyclopropylethylamino]methyl}-2-oxo-1,2-dihydronicotinamide

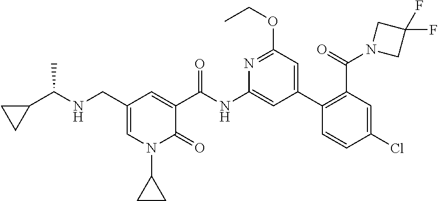 [SMILES: CCOc1cc(-c2ccc(Cl)cc2C(=O)N2CC(F)(F)C2)cc(NC(=O)c2cc(CN[C@@H](C)C3CC3)cn(C3CC3)c2=O)n1]

The title compound was prepared in a similar fashion to that described in Example 121 using (S)-1-cyclopropylethan-1-amine hydrochloride in step e. ¹H NMR (400 MHz, CDCl₃) δ 12.19 (s, 1H), 8.56 (dd, J=2.7, 1.0 Hz, 1H), 8.39 (s, 1H), 7.98 (t, J=1.2 Hz, 1H), 7.92-7.87 (m, 1H), 7.49 (t, J=1.5 Hz, 3H), 6.55 (t, J=1.2 Hz, 1H), 4.44-4.30 (m, 4H), 4.05-3.93 (m, 2H), 3.83 (d, J=13.2 Hz, 2H), 3.43 (tt, J=7.4, 4.1 Hz, 1H), 2.24-2.15 (m, 1H), 1.37 (td, J=7.1, 1.0 Hz, 3H), 1.29-1.14 (m, 5H), 0.98 (dd, J=6.5, 4.4 Hz, 2H), 0.92-0.85 (m, 1H), 0.62 (p, J=8.0, 7.4 Hz, 1H), 0.53 (tt, J=9.2, 5.0 Hz, 1H), 0.31 (dq, J=9.8, 5.1 Hz, 1H), 0.11 (dq, J=10.1, 5.2 Hz, 1H). ESI MS [M+H]⁺ for C₃₂H₃₅ClF₂N₅O₄, calcd 626.2, found 626.3.

### Example 125: N-(4-{4-cyano-2-[(3,3-difluoro-1-azetidinyl)carbonyl]-6-fluorophenyl}-6-cyclopropyl-2-pyridyl)-1-cyclopropyl-5-{[(1S,2S)-2-methoxy-1-methylpropylamino]methyl}-2-oxo-1,2-dihydronicotinamide

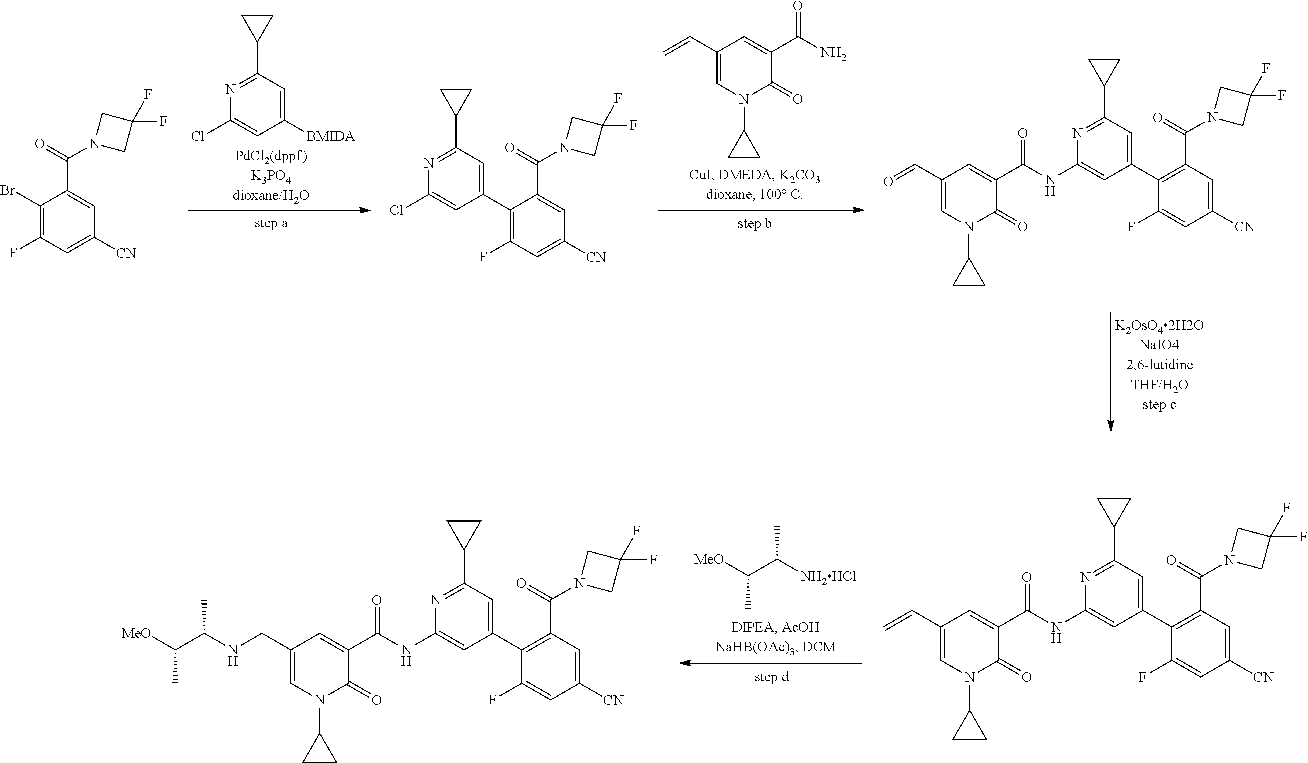

Step a: A 40 mL vial was charged with 4-bromo-3-(3,3-difluoroazetidine-1-carbonyl)-5-fluorobenzonitrile (1.0 g, 3.1 mmol. 1 equiv., prepared according to example 101), 1-(2-chloro-6-cyclopropylpyridin-4-yl)-5-methyl-2,8-dioxa-5-azonia-1-boranuidabicyclo[3.3.0]octane-3,7-dione (1.3 g, 4.1 mmol, 1.1 equiv.), and K₃PO₄ (2.0 g, 9.4 mmol, 3 equiv.). The reagents were suspended in a mixture of dioxane/water (16 mL, 4:1 v/v, 0.1 M), and the resulting solution was sparged with N₂ for 10 minutes. Then Pd(dppf)Cl₂ (230 mg, 0.3 mmol, 10 mol %) was added, and the mixture was heated to 95° C. for 3 hours. The reaction mixture was partitioned between EtOAc (50 mL) and water (50 mL). The organic phase was separated, and the aqueous phase was additionally extracted with EtOAc (3×50 mL). The combined organics were dried over Na₂SO₄ and concentrated to dryness under reduced pressure. The crude residue was purified via column chromatography (SiO₂, 0-50% EtOAc gradient in hexane) to afford the desired product.

Step b: To a suspension of the product from step a (468 mg, 1.2 mmol, 1.0 equiv.), 1-cyclopropyl-5-ethenyl-2-oxopyridine-3-carboxamide (246 g, 1.2 mmol, 1.0 equiv.), and K₂CO₃ (994 mg, 7.2 mmol, 6.0 equiv.) in dioxane (24 mL) was added CuI (457 mg, 2.4 mmol, 2.0 equiv.) and DMEDA (212 mg, 2.4 mmol, 2.0 equiv.). The reaction mixture was heated to 110° C. and stirred for 16 hours, at which point it was quenched with a 1:1 mixture of water/brine (50 mL) and extracted with EtOAc (2×75 mL). The combined organics were dried over Na₂SO₄, filtered, and concentrated under a vacuum. The crude residue was purified by column chromatography (SiO₂, EtOAc in hexanes, 0 to 100%) to afford the desired product.

Step c: To a solution of the product from step c (0.29 g, 0.5 mmol, 1.0 equiv.) in a 1:1 mixture of THF/H₂O (22 mL) was added 2,6-lutidine (111 mg, 1.0 mmol, 2.0 equiv.) followed by NaIO₄ (0.44 g, 2.1 mmol, 4.0 equiv.) and K₂OsO₄*2H₂O (19.2 mg, 0.05 mmol, 0.1 equiv.). The reaction mixture was stirred for 4 hours at room temperature, at which point it was quenched with saturated aqueous Na₂S₂O₃ (20 mL) and extracted with EtOAc (2×50 mL). The combined organics were dried over Na₂SO₄, filtered, and concentrated under a vacuum. The crude residue was used directly without further purification.

Step d: To a solution of the crude product from step c (˜87 μmol) in DCM (1.0 mL) was added (2S,3S)-3-methoxybutan-2-amine hydrochloride (36 mg, 0.3 mmol, 3 equiv.) and DIPEA (75 μL, 0.5 mmol, 5.0 equiv.). The resulting mixture was stirred at room temperature for 30 min before NaBH(OAc)₃ (37 mg, 0.2 mmol, 2 equiv.) and AcOH (37 μL, 0.5 mmol, 5 equiv. was added. The reaction mixture was then stirred at room temperature for overnight. The mixture was then diluted with EtOAc, washed with water and brine, dried over Na₂SO₄, and concentrated. The crude residue was purified by HPLC to afford the title compound. ¹H NMR (400 MHz, CDCl₃) δ 12.32 (s, 1H), 8.53 (d, J=2.7 Hz, 1H), 8.20 (t, J=1.4 Hz, 1H), 7.69 (d, J=2.6 Hz, 1H), 7.59 (d, J=0.9 Hz, 1H), 7.55 (dd, J=8.8, 1.6 Hz, 1H), 6.96 (t, J=1.5 Hz, 1H), 4.30 (t, J=11.8 Hz, 2H), 4.14 (t, J=11.4 Hz, 2H), 3.75 (d, J=13.5 Hz, 1H), 3.56 (d, J=13.6 Hz, 1H), 3.46 (tt, J=7.5, 4.2 Hz, 1H), 3.34 (s, 3H), 3.19-3.14 (m, 2H), 2.63 (q, J=6.7 Hz, 2H), 2.00 (td, J=8.2, 4.1 Hz, 1H), 1.26 (dt, J=7.3, 3.7 Hz, 2H), 1.13 (dd, J=10.3, 5.5 Hz, 4H), 1.06 (d, J=6.3 Hz, 3H), 0.97 (dq, J=6.9, 3.8 Hz, 4H). ESI MS [M+H]⁺ for C₃₄H₃₆F₃N₆O₄, calcd 649.3, found 649.3.

### Example 126: N-(4-{4-cyano-2-[(3,3-difluoro-1-azetidinyl)carbonyl]-6-fluorophenyl}-6-isopropoxy-2-pyridyl)-5-{[((S)-1-cyclobutylethylamino]methyl}-1-cyclopropyl-2-oxo-1,2-dihydronicotinamide

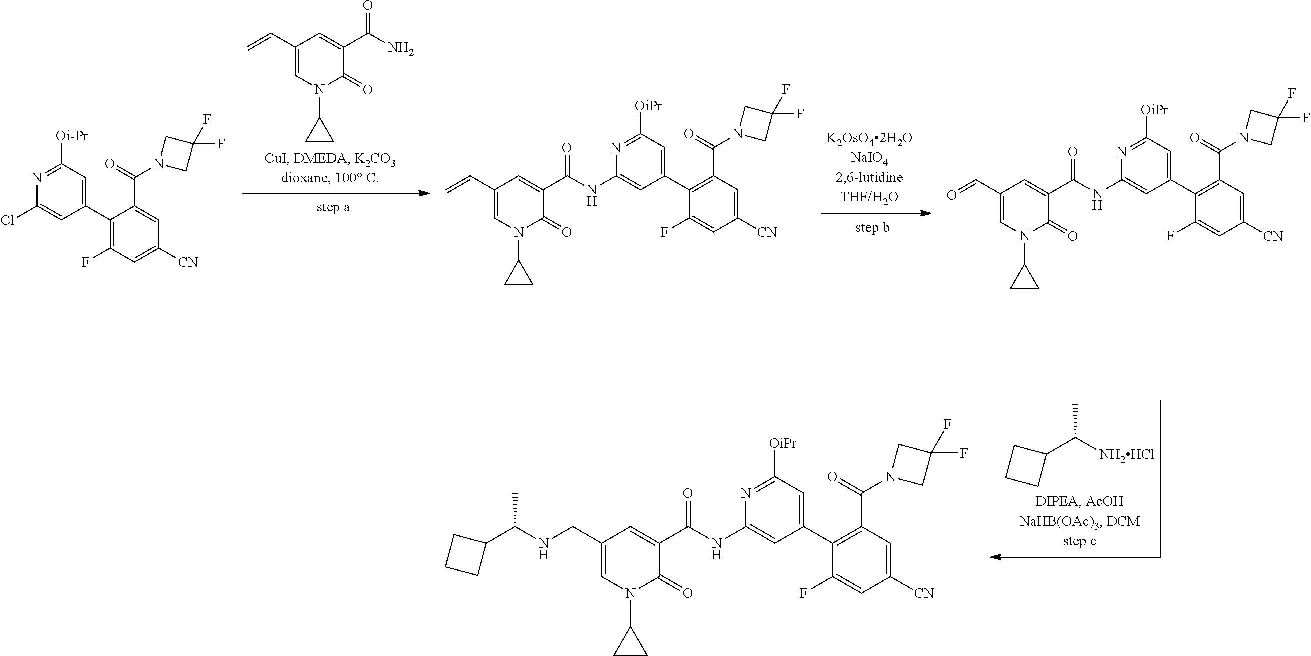

Step a: To a suspension of 4-(2-chloro-6-propan-2-yloxypyridin-4-yl)-3-(3,3-difluoroazetidine-1-carbonyl)-5-fluorobenzonitrile (259 mg, 0.6 mmol, 1.0 equiv., prepared according to Example 101), 1-cyclopropyl-5-ethenyl-2-oxopyridine-3-carboxamide (130 mg, 0.6 mmol, 1.0 equiv.), and K₂CO₃ (523 mg, 3.8 mmol, 6.0 equiv.) in dioxane (13 mL) was added CuI (240 mg, 1.3 mmol, 2.0 equiv.) and DMEDA (111 mL, 1.3 mmol, 2.0 equiv.). The reaction mixture was heated to 100° C. and stirred for 72 hours, at which point it was quenched with a 1:1 mixture of water/brine (50 mL) and extracted with EtOAc (2×75 mL). The combined organics were dried over Na₂SO₄, filtered, and concentrated under a vacuum. The crude residue was purified by column chromatography (SiO₂, EtOAc in hexanes, 0 to 100%) to afford the desired product.

Step b: To a solution of the product from step a (180 g, 0.3 mmol, 1.0 equiv.) in a 1:1 mixture of THF/H₂O (3.2 mL) was added 2,6-lutidine (67 mg, 0.62 mmol, 2.0 equiv.) followed by NaIO₄ (667 mg, 3.1 mmol, 10.0 equiv.) and K₂OsO₄*2H₂O (11 mg, 0.03 mmol, 0.1 equiv.). The reaction mixture was stirred for 3 hours at room temperature, at which point it was quenched with saturated aqueous Na₂S₂O₃ (20 mL) and extracted with EtOAc (2×50 mL). The combined organics were dried over Na₂SO₄, filtered, and concentrated under a vacuum. The crude residue was used directly without further purification.

Step c: To a solution of the crude product from step b (˜73 μmol) in DCM (1.0 mL) was added (1S)-1-cyclobutylethan-1-amine hydrochloride (20 mg, 0.15 mmol, 2 equiv.) and DIPEA (25 mg, 0.15 mmol, 2 equiv.). The resulting mixture was stirred at room temperature for 30 min before NaBH(OAc)₃ (31 mg, 0.15 mmol, 2 equiv.) and AcOH (12 μL, 0.15 mmol, 2 equiv. was added. The reaction mixture was then stirred at room temperature for overnight. The mixture was then diluted with EtOAc, washed with water and brine, dried over Na₂SO₄, and concentrated. The crude residue was purified by HPLC to afford the title compound. ¹H NMR (400 MHz, CDCl₃)¹H NMR (400 MHz, Chloroform-d) δ 12.24 (s, 1H), 8.53 (d, J=2.6 Hz, 1H), 7.97 (s, 1H), 7.69 (d, J=2.6 Hz, 1H), 7.59 (s, 1H), 7.54 (dd, J=8.8, 1.5 Hz, 1H), 6.51 (d, J=1.6 Hz, 1H), 5.45 (p, J=6.2 Hz, 1H), 4.32 (t, J=11.8 Hz, 2H), 4.17 (t, J=11.4 Hz, 2H), 3.71 (d, J=13.4 Hz, 1H), 3.59 (d, J=13.4 Hz, 1H), 3.47 (tt, J=7.6, 4.2 Hz, 1H), 2.67-2.55 (m, 1H), 2.27 (p, J=8.4 Hz, 1H), 2.09-2.01 (m, 2H), 1.85 (d, J=8.0 Hz, 1H), 1.72 (td, J=15.0, 14.3, 8.5 Hz, 4H), 1.34 (d, J=6.2 Hz, 6H), 1.25 (dd, J=7.6, 5.5 Hz, 2H), 0.99 (dd, J=8.4, 5.3 Hz, 5H). ESI MS [M+H]⁺ for C₃₅H₃₈F₃N₆O₄, calcd 663.3, found 663.2.

### Example 127: N-(4-{4-cyano-2-[(3,3-difluoro-1-azetidinyl)carbonyl]-6-fluorophenyl}-6-isopropoxy-2-pyridyl)-1-cyclopropyl-5-{[(S)-2-methoxypropylamino]methyl}-2-oxo-1,2-dihydronicotinamide

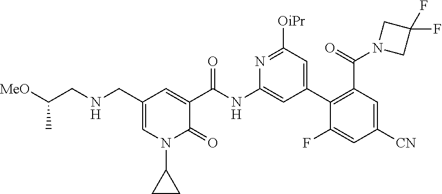 [SMILES: CO[C@@H](C)CNCc1cc(C(=O)Nc2cc(-c3c(F)cc(C#N)cc3C(=O)N3CC(F)(F)C3)cc(OC(C)C)n2)c(=O)n(C2CC2)c1]

The title compound was prepared in a similar fashion to that described in Example 126 using (2S)-2-methoxypropan-1-amine in step c. ¹H NMR (400 MHz, CDCl₃) δ 12.20 (s, 1H), 8.58 (d, J=2.6 Hz, 1H), 8.00 (s, 1H), 7.84 (d, J=2.5 Hz, 1H), 7.53-7.43 (m, 3H), 6.56 (d, J=1.3 Hz, 1H), 4.45-4.30 (m, 4H), 4.10 (d, J=7.5 Hz, 1H), 4.00 (t, J=11.6 Hz, 2H), 3.95-3.80 (m, 3H), 3.74 (q, J=7.2 Hz, 1H), 3.50-3.39 (m, 1H), 2.94 (d, J=11.1 Hz, 1H), 2.82-2.72 (m, 1H), 2.02 (dq, J=13.0, 6.7 Hz, 1H), 1.90 (p, J=7.0 Hz, 2H), 1.62-1.50 (m, 2H), 1.38 (t, J=7.1 Hz, 3H), 1.23 (q, J=6.9 Hz, 2H), 0.98 (d, J=4.6 Hz, 2H). ESI MS [M+H]⁺ for C₃₃H₃₆F₃N₆O₅, calcd 642.2, found 642.2.

### Example 128: N-(4-{4-cyano-2-[(3,3-difluoro-1-azetidinyl)carbonyl]-6-fluorophenyl}-6-isopropoxy-2-pyridyl)-1-cyclopropyl-5-[(isobutylamino)methyl]-2-oxo-1,2-dihydronicotinamide

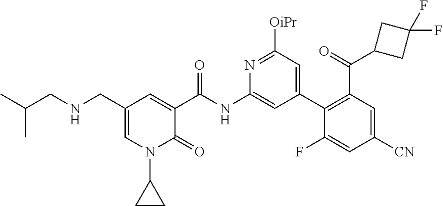 [SMILES: CC(C)CNCc1cc(C(=O)Nc2cc(-c3c(F)cc(C#N)cc3C(=O)C3CC(F)(F)C3)cc(OC(C)C)n2)c(=O)n(C2CC2)c1]

The title compound was prepared in a similar fashion to that described in Example 126 using isobutylamine in step c. ¹H NMR (400 MHz, CDCl₃) δ 12.26 (s, 1H), 8.54 (d, J=2.7 Hz, 1H), 7.98 (d, J=1.5 Hz, 1H), 7.67 (s, 1H), 7.61-7.50 (m, 2H), 6.51 (d, J=1.5 Hz, 1H), 5.45 (p, J=6.1 Hz, 1H), 4.32 (t, J=11.8 Hz, 2H), 4.17 (t, J=11.3 Hz, 2H), 3.65 (s, 2H), 3.47 (tt, J=7.7, 4.3 Hz, 1H), 2.44 (d, J=6.8 Hz, 2H), 1.76 (dt, J=13.3, 6.7 Hz, 3H), 1.34 (d, J=6.1 Hz, 5H), 1.26 (q, J=6.9 Hz, 2H), 0.98 (d, J=4.3 Hz, 2H), 0.93 (d, J=6.7 Hz, 5H). ESI MS [M+H]⁺ for C₃₃H₃₅F₃N₆O₄, calcd 637.3, found 637.3.

### Example 129: N-(4-{4-cyano-2-[(3,3-difluoro-1-azetidinyl)carbonyl]phenyl}-6-(2,2-difluoroethoxy)-2-pyridyl)-1-cyclopropyl-5-[(isobutylamino)methyl]-2-oxo-1,2-dihydronicotinamide

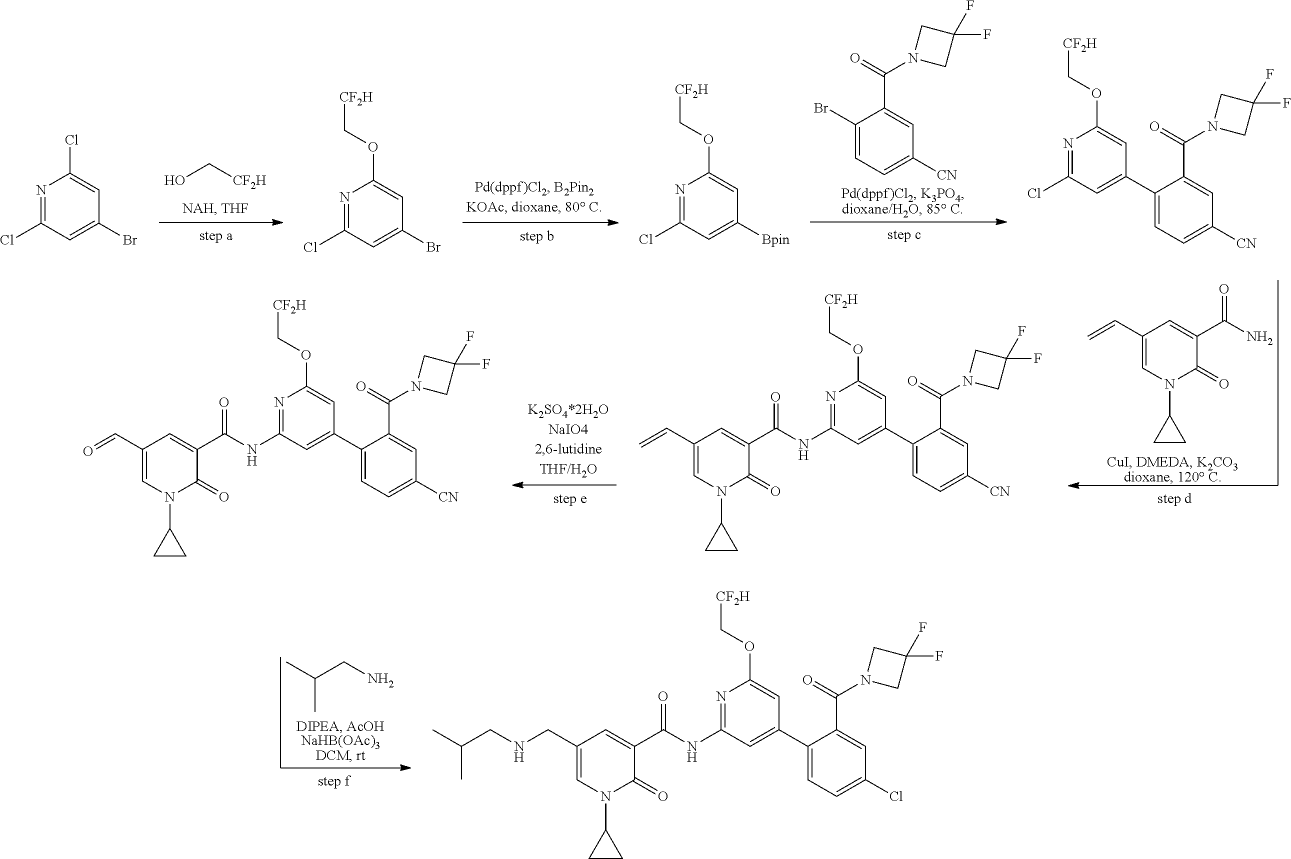

Step a: To a solution of 2,2-difluoroethanol (2.2 g, 21.2 mmol, 1.5 equiv.) in THF (88 mL, 0.2 M) was added NaH (1.8 g, 60% in mineral oil, 21.2 mmol, 1.5 equiv.) at 0° C. The cooling bath was removed, and the mixture was stirred at the room temperature for 10 minutes, until all NaH was dissolved. 4-bromo-2,6-dichloropyridine (4.0 g, 17.6 mmol, 1.0 equiv.) was added to the reaction mixture and stirred for 17 h at room temperature. Then, the reaction mixture was quenched with water (100 mL), diluted with EtOAc (200 mL), washed with sat. aq. NH₄Cl (100 ml), and then water (100 mL). The organic phase was dried over Na₂SO₄, concentrated and the crude residue was purified by column chromatography (SiO₂, EtOAc in hexanes, 0 to 15%) to give the desired product.

Step b: A 40 mL vial was charged with the product from step a (3.5 g, 12.8 mmol, 1.1 equiv.), bis(pinacolato)diboron (3.6 g, 14.1 mmol, 1.1 equiv.), KOAc (4.4 g, 44.8 mmol, 3.5 equiv.), Pd(dppf)Cl₂ (936 mg, 1.3 mmol, 0.1 equiv.), and dry dioxane (64 mL, 0.2 M) was added, the reaction mixture was sparged with N₂ for 10 minutes and heated to 80° C. for 2 h. The resulting solution was cooled to ambient temperature and diluted with brine (100 mL) and EtOAc (75 mL). The organic phase was separated and the aqueous phase was additionally extracted with EtOAc (75 mL). The combined organic extract was dried over Na₂SO₄, filtered, and concentrated to dryness under reduced pressure. The crude residue was used without further purification.

Step c: To a solution of the crude product of step b (˜2.56 mmol, 1.0 equiv.), 4-bromo-3-(3,3-difluoroazetidine-1-carbonyl)benzonitrile (768 mg, 2.56 mmol, 1.0 equiv., prepared according to Example 96), and K₃PO₄ (1.7 g, 7.7 mmol, 3.0 equiv.) in dioxane (10 mL, 0.2 M)/H₂O (2.5 mL, 0.2 M) was added Pd(dppf)Cl₂ (187 g, 0.3 mmol, 0.1 equiv.) at rt under nitrogen atmosphere. The resulting mixture was stirred for 3 h at 85° C. The mixture was allowed to cool down to rt. The reaction was quenched with sat. aq. NH₄Cl solution, the organic phase was separated, and the aqueous layer was extracted with EtOAc. The combined organic phase was dried over Na₂SO₄, concentrated and the crude residue was purified by column chromatography (SiO₂, hexane in EtOAc, 0 to 60%) to afford the desired product.

Step d: To a mixture of the product of step c (348 mg, 0.8 mmol, 1.0 equiv.) and 1-cyclopropyl-5-ethenyl-2-oxopyridine-3-carboxamide (173 mg, 0.8 mmol, 1.0 equiv.) in dioxane (17 mL) was added CuI (319 mg, 1.7 mmol, 2.0 equiv.), N,N′-dimethylethylenediamine (148 mg, 1.7 mmol, 2.0 equiv.) and K₂CO₃ (712 mg, 5.2 mmol, 6.0 equiv.) The resulting mixture was heated at 120° C. overnight. The reaction mixture was cooled to room temperature, diluted with EtOAc, washed with H₂O and brine, filtered through Na₂SO₄, and concentrated. The crude product was purified by column chromatography (SiO₂, 0-80% EtOAc gradient in hexanes).

Step e: To a solution of the product from step d (0.27 g, 0.47 mmol, 1.0 equiv.) in a 1:1 mixture of THF/H₂O (5 mL) was added 2,6-lutidine (0.1 mL, 0.93 mmol, 2.0 equiv.) followed by NaIO₄ (995 mg, 4.7 mmol, 10.0 equiv.) and K₂OsO₄*2H₂O (17 mg, 0.05 mmol, 0.1 equiv.). The reaction mixture was stirred for 4 hours at room temperature, at which point it was quenched with saturated aqueous Na₂S₂O₃ (10 mL) and extracted with EtOAc (2×20 mL). The combined organics were dried over Na₂SO₄, filtered, and concentrated under a vacuum. The crude residue was used directly without further purification.

Step f: To a solution of the crude product from step e (˜69 μmol) in DCM (1.0 mL) was added isobutylamine (10 mg, 0.14 mmol, 1 equiv.) and DIPEA (24 μL, 0.14 mmol, 2 equiv.). The resulting mixture was stirred at room temperature for 30 min before NaBH(OAc)₃ (29 mg, 0.14 mmol, 2 equiv.) and AcOH (12 μL, 0.14 mmol, 2 equiv.) was added. The reaction mixture was then stirred at room temperature for overnight. The mixture was then diluted with EtOAc, washed with water and brine, dried over Na₂SO₄, and concentrated. The crude residue was purified by HPLC to afford the title compound. ¹H NMR (400 MHz, CDCl₃) δ 12.43 (s, 1H), 8.56 (d, J=2.6 Hz, 1H), 8.16 (d, J=1.4 Hz, 1H), 7.85-7.78 (m, 2H), 7.70-7.63 (m, 2H), 6.67 (d, J=1.4 Hz, 1H), 6.13 (tt, J=55.4, 4.1 Hz, 1H), 4.60 (td, J=13.6, 4.2 Hz, 2H), 4.40 (t, J=11.8 Hz, 2H), 4.10 (dt, J=12.0, 7.9 Hz, 2H), 3.65 (s, 2H), 3.48 (tt, J=7.6, 4.3 Hz, 1H), 2.43 (d, J=6.7 Hz, 2H), 1.74 (dh, J=13.4, 6.6 Hz, 1H), 1.31-1.24 (m, 3H), 1.00-0.95 (m, 2H), 0.93 (d, J=6.6 Hz, 6H). ESI MS [M+H]⁺ for C₃₂H₃₃F₄N₆O₄, calcd 641.2, found 641.3.

### Example 130: N-(4-{4-cyano-2-[(3,3-difluoro-1-azetidinyl)carbonyl]phenyl}-6-(2,2-difluoroethoxy)-2-pyridyl)-5-{[(S)-1-cyclobutylethylamino]methyl}-1-cyclopropyl-2-oxo-1,2-dihydronicotinamide

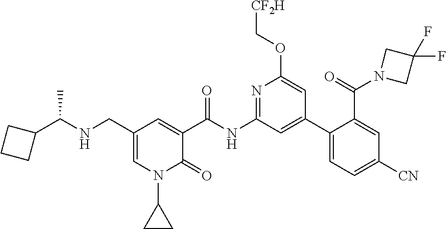 [SMILES: C[C@H](NCc1cc(C(=O)Nc2cc(-c3ccc(C#N)cc3C(=O)N3CC(F)(F)C3)cc(OCC(F)F)n2)c(=O)n(C2CC2)c1)C1CCC1]

The title compound was prepared in a similar fashion to that described in Example 129 using (1S)-1-cyclobutylethan-1-amine hydrochloride in step f. ¹H NMR (400 MHz, CDCl₃) δ 12.42 (s, 1H), 8.55 (d, J=2.6 Hz, 1H), 8.16 (d, J=1.3 Hz, 1H), 7.85-7.78 (m, 2H), 7.71-7.62 (m, 2H), 6.67 (d, J=1.2 Hz, 1H), 6.12 (tt, J=55.2, 4.1 Hz, 2H), 4.60 (td, J=13.6, 4.2 Hz, 2H), 4.39 (t, J=11.8 Hz, 2H), 4.08 (t, J=11.4 Hz, 2H), 3.70 (d, J=13.5 Hz, 1H), 3.58 (d, J=13.6 Hz, 1H), 3.47 (tt, J=7.6, 4.3 Hz, 1H), 2.59 (q, J=6.9 Hz, 1H), 2.23 (q, J=8.1 Hz, 1H), 2.08-1.95 (m, 2H), 1.87 (q, J=8.6, 8.2 Hz, 1H), 1.79-1.67 (m, 4H), 1.28 (t, J=6.9 Hz, 2H), 0.98 (d, J=6.0 Hz, 4H). ESI MS [M+H]⁺ for C₃₄H₃₅F₄N₆O₄, calcd 667.3, found 667.3.

### Example 131: N-(4-{4-cyano-2-[(3,3-difluoro-1-azetidinyl)carbonyl]phenyl}-6-(2,2-difluoroethoxy)-2-pyridyl)-1-cyclopropyl-5-{[(S)-2-methoxypropylamino]methyl}-2-oxo-1,2-dihydronicotinamide

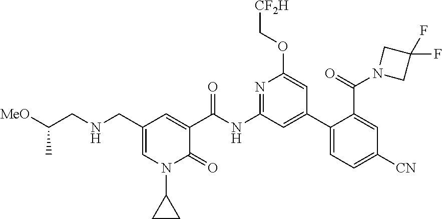 [SMILES: CO[C@@H](C)CNCc1cc(C(=O)Nc2cc(-c3ccc(C#N)cc3C(=O)N3CC(F)(F)C3)cc(OCC(F)F)n2)c(=O)n(C2CC2)c1]

The title compound was prepared in a similar fashion to that described in Example 129 using (2S)-2-methoxypropan-1-amine in step f. ¹H NMR (400 MHz, CDCl₃) δ 12.42 (s, 1H), 8.55 (d, J=2.7 Hz, 1H), 8.15 (d, J=1.4 Hz, 1H), 7.81 (d, J=7.4 Hz, 2H), 7.70-7.62 (m, 2H), 6.66 (d, J=1.4 Hz, 1H), 6.12 (tt, J=55.4, 4.2 Hz, 1H), 4.60 (td, J=13.6, 4.2 Hz, 2H), 4.44-4.33 (m, 2H), 4.17-4.02 (m, 2H), 3.72-3.59 (m, 2H), 3.51-3.43 (m, 2H), 3.35 (s, 3H), 2.68-2.55 (m, 2H), 1.30-1.24 (m, 3H), 1.15 (d, J=6.2 Hz, 3H), 1.01-0.95 (m, 2H). ESI MS [M+H]⁺ for C₃₂H₃₃F₄N₆O₅, calcd 657.2, found 657.3.

### Example 132: N-(4-{4-cyano-2-[(3,3-difluoro-1-azetidinyl)carbonyl]phenyl}-6-(2,2-difluoroethoxy)-2-pyridyl)-1-cyclopropyl-2-oxo-5-[({[(S)-perhydro-2-furyl]methyl}amino)methyl]-1,2-dihydronicotinamide

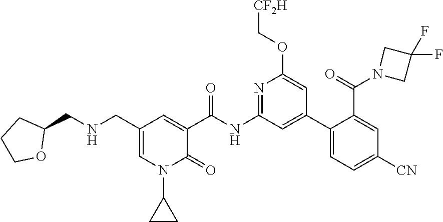 [SMILES: N#Cc1ccc(-c2cc(NC(=O)c3cc(CNC[C@@H]4CCCO4)cn(C4CC4)c3=O)nc(OCC(F)F)c2)c(C(=O)N2CC(F)(F)C2)c1]

The title compound was prepared in a similar fashion to that described in Example 129 using (S)-tetrahydrofurfurylamine in step f. ¹H NMR (400 MHz, CDCl₃) δ 12.43 (s, 1H), 8.55 (d, J=2.6 Hz, 1H), 8.16 (d, J=1.4 Hz, 1H), 7.85-7.78 (m, 2H), 7.72-7.63 (m, 2H), 6.67 (d, J=1.4 Hz, 1H), 6.12 (tt, J=55.3, 4.1 Hz, 1H), 4.60 (td, J=13.6, 4.2 Hz, 2H), 4.40 (t, J=11.9 Hz, 2H), 4.11-3.96 (m, 3H), 3.86 (dt, J=8.1, 6.6 Hz, 1H), 3.81-3.73 (m, 1H), 3.69 (s, 2H), 3.47 (tt, J=7.5, 4.2 Hz, 1H), 2.74 (dd, J=12.0, 3.5 Hz, 1H), 2.64 (dd, J=12.0, 7.7 Hz, 1H), 2.02-1.96 (m, 1H), 1.96-1.83 (m, 2H), 1.64-1.50 (m, 1H), 1.33-1.23 (m, 4H), 1.03-0.94 (m, 2H). ESI MS [M+H]⁺ for C₃₃H₃₃F₄N₆O₅, calcd 669.3, found 669.3.

### Example 133: N-(4-{4-chloro-2-[(3,3-difluoro-1-azetidinyl)carbonyl]phenyl}-6-cyclopropyl-2-pyridyl)-1-cyclopropyl-5-[({[(R)-1,4-dioxan-2-yl]methyl}amino)methyl]-2-oxo-1,2-dihydronicotinamide

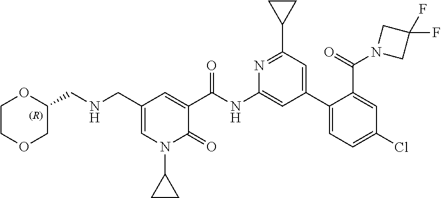

The title compound was prepared in a similar fashion to that described for Example 90 using N-[4-[4-chloro-2-(3,3-difluoroazetidine-1-carbonyl)phenyl]-6-cyclopropylpyridin-2-yl]-1-cyclopropyl-5-formyl-2-oxopyridine-3-carboxamide and [(2R)-1,4-dioxan-2-yl]methanamine in step e. ¹H NMR (400 MHz, Chloroform-d) δ 12.27 (s, 1H), 8.54 (d, J=2.6 Hz, 1H), 8.24 (d, J=1.5 Hz, 1H), 7.63 (d, J=2.6 Hz, 1H), 7.53-7.47 (m, 3H), 6.97 (d, J=1.5 Hz, 1H), 4.33 (t, J=11.9 Hz, 2H), 3.96 (t, J=11.6 Hz, 2H), 3.83-3.68 (m, 5H), 3.67-3.54 (m, 3H), 3.50-3.35 (m, 2H), 2.67-2.58 (m, 2H), 2.05-1.96 (m, 1H), 1.29-1.23 (m, 2H), 1.13-1.07 (m, 2H), 1.00-0.92 (m, 4H). ESI MS [M+H]⁺ for C₃₃H₃₄F₂N₅O₅ calcd 654.2 found 654.2.

### Example 134: N-(4-{4-chloro-2-[(3,3-difluoro-1-azetidinyl)carbonyl]phenyl}-6-cyclopropyl-2-pyridyl)-1-cyclopropyl-5-[(isobutylamino)methyl]-2-oxo-1,2-dihydronicotinamide

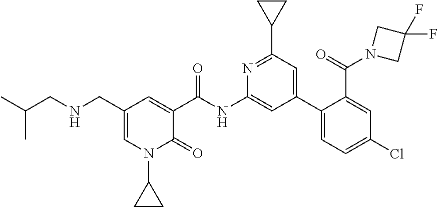 [SMILES: CC(C)CNCc1cc(C(=O)Nc2cc(-c3ccc(Cl)cc3C(=O)N3CC(F)(F)C3)cc(C3CC3)n2)c(=O)n(C2CC2)c1]

The title compound was prepared in a similar fashion to that described for Example 90 using N-[4-[4-chloro-2-(3,3-difluoroazetidine-1-carbonyl)phenyl]-6-cyclopropylpyridin-2-yl]-1-cyclopropyl-5-formyl-2-oxopyridine-3-carboxamide and isobutylamine in step e. ¹H NMR (400 MHz, Chloroform-d) δ 12.26 (s, 1H), 8.55 (d, J=2.6 Hz, 1H), 8.23 (d, J=1.5 Hz, 1H), 7.71 (d, J=2.6 Hz, 1H), 7.55-7.45 (m, 3H), 6.97 (d, J=1.5 Hz, 1H), 4.33 (t, J=11.9 Hz, 2H), 3.96 (t, J=11.6 Hz, 2H), 3.70 (s, 2H), 3.46 (tt, J=7.6, 4.2 Hz, 1H), 2.48 (d, J=6.8 Hz, 2H), 2.19 (s, 1H), 2.04-1.96 (m, 1H), 1.86-1.74 (m, 1H), 1.26 (t, J=7.1 Hz, 2H), 1.13-1.06 (m, 2H), 0.99-0.89 (m, 10H). ESI MS [M+H]⁺ for C₃₂H₃₄ClF₂N₅O₃ calcd 609.2 found 610.2.

### Example 135: N-(4-{4-chloro-2-[(3,3-difluoro-1-azetidinyl)carbonyl]phenyl}-6-cyclopropyl-2-pyridyl)-1-cyclopropyl-5-{[(S)-1-cyclopropylethylamino]methyl}-2-oxo-1,2-dihydronicotinamide

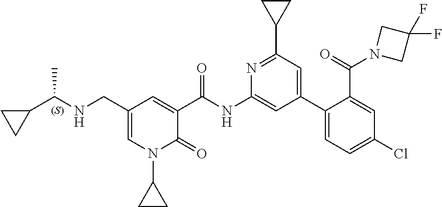

The title compound was prepared in a similar fashion to that described for Example 90 using N-[4-[4-chloro-2-(3,3-difluoroazetidine-1-carbonyl)phenyl]-6-cyclopropylpyridin-2-yl]-1-cyclopropyl-5-formyl-2-oxopyridine-3-carboxamide and (1S)-1-cyclopropylethanamine in step e. ¹H NMR (400 MHz, Chloroform-d) δ 12.22 (s, 1H), 8.58 (d, J=2.6 Hz, 1H), 8.18 (d, J=1.4 Hz, 1H), 7.90 (s, 1H), 7.50 (d, J=1.5 Hz, 3H), 6.97 (d, J=1.5 Hz, 1H), 4.33 (t, J=11.9 Hz, 2H), 4.02-3.89 (m, 3H), 3.86-3.77 (m, 1H), 3.48-3.38 (m, 1H), 2.22-2.15 (m, 1H), 2.03-1.96 (m, 1H), 1.28 (d, J=6.4 Hz, 3H), 1.25-1.18 (m, 2H), 1.12-1.05 (m, 2H), 1.03-0.96 (m, 2H), 0.97-0.92 (m, 2H), 0.65-0.48 (m, 2H), 0.35-0.27 (m, 1H), 0.16-0.05 (m, 2H). ESI MS [M+H]⁺ for C₃₃H₃₄ClF₂N₅O₃ calcd 622.2 found 622.2.

### Example 136: N-(4-{4-chloro-2-[(3,3-difluoro-1-azetidinyl)carbonyl]phenyl}-6-cyclopropyl-2-pyridyl)-1-cyclopropyl-5-[(3-fluoropropylamino)methyl]-2-oxo-1,2-dihydronicotinamide

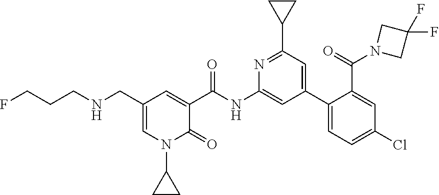 [SMILES: O=C(Nc1cc(-c2ccc(Cl)cc2C(=O)N2CC(F)(F)C2)cc(C2CC2)n1)c1cc(CNCCCF)cn(C2CC2)c1=O]

The title compound was prepared in a similar fashion to that described for Example 90 using N-[4-[4-chloro-2-(3,3-difluoroazetidine-1-carbonyl)phenyl]-6-cyclopropylpyridin-2-yl]-1-cyclopropyl-5-formyl-2-oxopyridine-3-carboxamide and 3-fluoropropan-1-amine in step e. ¹H NMR (400 MHz, Chloroform-d) δ 12.25 (s, 1H), 8.55 (d, J=2.7 Hz, 1H), 8.22 (d, J=1.5 Hz, 1H), 7.69 (d, J=2.7 Hz, 1H), 7.54-7.46 (m, 3H), 6.97 (d, J=1.5 Hz, 1H), 4.60 (t, J=5.7 Hz, 1H), 4.48 (t, J=5.7 Hz, 1H), 4.33 (t, J=11.9 Hz, 2H), 3.96 (t, J=11.6 Hz, 2H), 3.72 (s, 2H), 3.51-3.40 (m, 1H), 2.84 (t, J=6.9 Hz, 2H), 2.05-1.84 (m, 3H), 1.30-1.21 (m, 2H), 1.13-1.06 (m, 2H), 1.02-0.90 (m, 4H). ESI MS [M+H]⁺ for C₃₁H₃₁ClF₃N₅O₃ calcd 614.2 found 614.2.

### Example 137: N-(4-{2-[(3,3-difluoro-1-azetidinyl)carbonyl]-4-fluorophenyl}-6-isopropoxy-2-pyridyl)-1-cyclopropyl-5-{[(S)-2-methoxypropylamino]methyl}-2-oxo-1,2-dihydronicotinamide

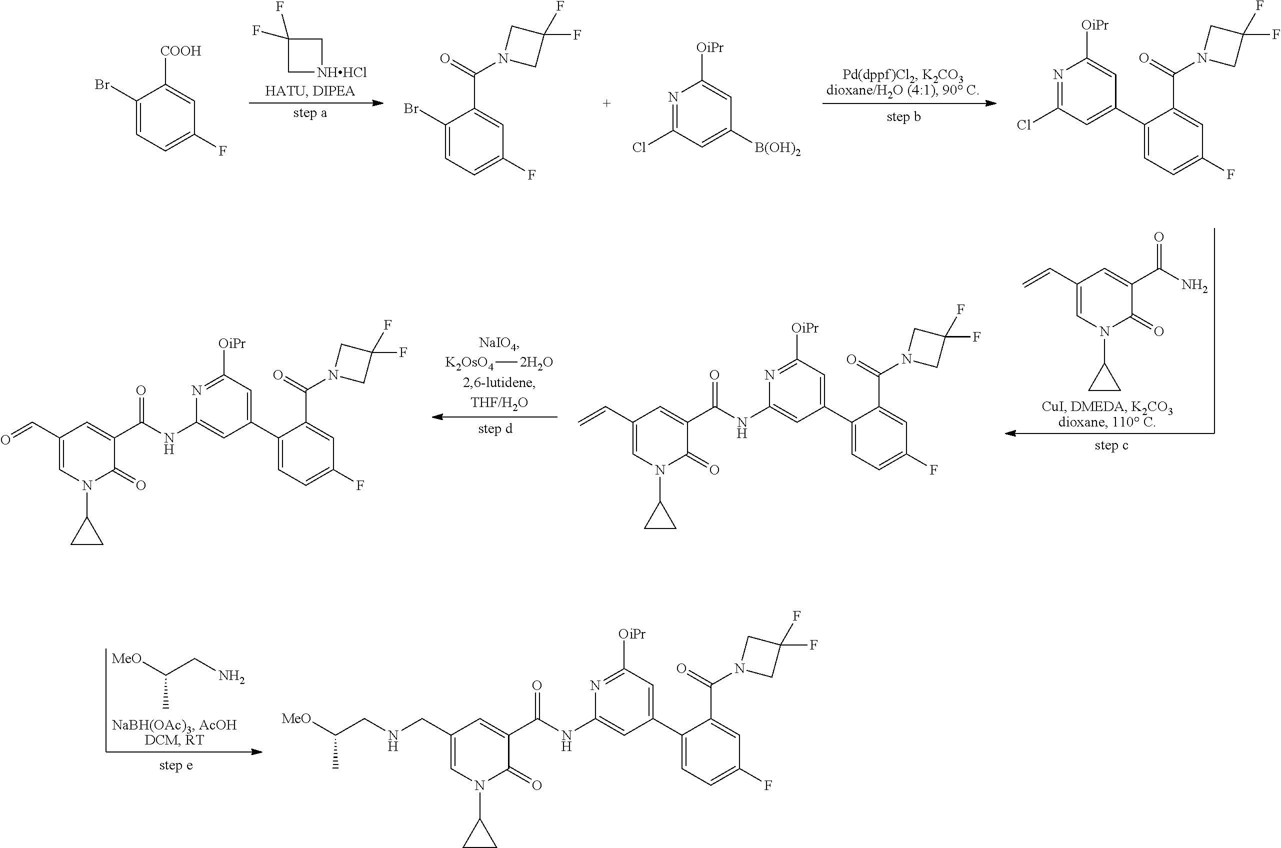

Step a: To a solution of a 2-bromo-5-fluorobenzoic acid (0.65 g, 3.0 mmol, 1 equiv.) and 3,3-difluoroazetidine hydrochloride (0.50 g, 3.9 mmol, 1.3 equiv.) in THF (15 mL, 0.2 M) was added DIPEA (1.06 mL, 6.0 mmol, 2 equiv) followed by HATU (1.7 g, 4.5 mmol, 1.5 equiv.). The reaction mixture was stirred for 16 hours at room temperature, at which point it was partitioned between EtOAc (50 mL) and water (10 mL). The aqueous layer was extracted with EtOAc (2×25 mL), and the combined organics were dried over Na₂SO₄, filtered, and concentrated under a vacuum. The crude residue was purified by column chromatography (SiO₂, 0-100% EtOAc in hexanes) to afford the desired product.

Step b: To a solution of the product of step a (0.58 g, 2.0 mmol, 1.0 equiv.), (2-chloro-6-propan-2-yloxypyridin-4-yl)boronic acid (0.43 g, 2.0 mmol, 1.0 equiv.) and K₂CO₃ (0.82 g, 6.0 mmol, 3.0 equiv.) in dioxane/H₂O (4:1, 10 mL, 0.2 M) was added Pd(dppf)Cl₂ (0.14 g, 0.2 mmol, 0.1 equiv.) at rt under nitrogen atmosphere. The resulting mixture was stirred for 4 h at 90° C. The mixture was allowed to cool down to rt. The reaction was quenched with sat. aq. NH₄Cl solution, the organic phase was separated, and the aqueous layer was extracted with EtOAc. The combined organic phase was dried over Na₂SO₄, concentrated and the crude residue was purified by column chromatography (SiO₂, hexane in EtOAc, 0 to 60%) to afford the desired product.

Step c: To a suspension of the product from step b (0.25 g, 0.64 mmol, 1.0 equiv.), 1-cyclopropyl-5-ethenyl-2-oxopyridine-3-carboxamide (0.15 g, 0.76 mmol, 1.2 equiv.), and K₂CO₃ (0.26 g, 1.92 mmol, 3.0 equiv.) in dioxane (12 mL) was added CuI (0.12 g, 0.64 mmol, 1.0 equiv.) and DMEDA (0.14 mL, 1.28 mmol, 2.0 equiv.). The reaction mixture was heated to 110° C. and stirred for 16 hours, at which point it was quenched with a 1:1 mixture of water/brine (10 mL) and extracted with EtOAc (2×75 mL). The combined organics were dried over Na₂SO₄, filtered, and concentrated under a vacuum. The crude residue was purified by column chromatography (SiO₂, EtOAc in hexanes, 0 to 100%) to afford the desired product.

Step d: To a solution of the product from step c (0.26 g, 0.5 mmol, 1.0 equiv.) in a 1:1 mixture of THF/H₂O (5 mL) was added 2,6-lutidene (0.11 mL, 1.0 mmol, 2.0 equiv.) followed by NaIO₄ (0.42 g, 2.0 mmol, 4.0 equiv.) and K₂OsO₄*2H₂O (18 mg, 0.05 mmol, 0.1 equiv.). The reaction mixture was stirred for 12 hours at room temperature, at which point it was quenched with saturated aqueous Na₂S₂O₃ (20 mL) and extracted with EtOAc (2×75 mL). The combined organics were dried over Na₂SO₄, filtered, and concentrated under a vacuum. The crude residue was used directly without further purification.

Step e: To a solution of the product from step d (26.8 mg, 0.05 mmol, 1.0 equiv.) in DCM (1.0 mL) was added (2S)-2-methoxypropan-1-amine (8.9 mg, 0.1 mmol, 2.0 equiv.) followed by sodium triacetoxyborohydride (30 mg, 0.13 mmol, 2.5 equiv.) and AcOH (8 L, 0.13 mmol, 2.5 equiv.). The reaction mixture was stirred for 12 hours at room temperature, at which point it was quenched with saturated aqueous NaHCO₃ (10 mL) and extracted with EtOAc (2×25 mL). The combined organics were dried over Na₂SO₄, filtered, and concentrated under a vacuum. The crude residue was purified by reverse phase prep-HPLC (C18 column, 5 to 80% MeCN/H₂O) to afford the desired product. ¹H NMR (400 MHz, Chloroform-d) δ 12.21 (s, 1H), 8.57 (d, J=2.6 Hz, 1H), 8.03 (d, J=1.4 Hz, 1H), 7.64 (d, J=2.6 Hz, 1H), 7.58-7.48 (m, 1H), 7.25-7.17 (m, 2H), 6.52 (d, J=1.3 Hz, 1H), 5.50-5.38 (m, 1H), 4.35 (t, J=11.9 Hz, 2H), 4.00 (t, J=11.6 Hz, 2H), 3.70-3.60 (m, 2H), 3.53-3.43 (m, 2H), 3.36 (s, 3H), 2.66-2.56 (m, 2H), 1.33 (d, J=6.1 Hz, 6H), 1.27-1.24 (m, 2H), 1.15 (d, J=6.1 Hz, 3H), 1.01-0.92 (m, 2H). ESI MS [M+H]⁺ for C₃₂H₃₆F₃N₅O₅, calcd 628.2, found 628.2.

### Example 138: N-(4-{2-[(3,3-difluoro-1-azetidinyl)carbonyl]-4-fluorophenyl}-6-isopropoxy-2-pyridyl)-1-cyclopropyl-2-oxo-5-[({[(S)-perhydro-2-furyl]methyl}amino)methyl]-1,2-dihydronicotinamide

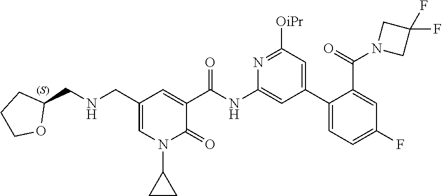

The title compound was prepared in a similar fashion to that described for Example 137 using 1-cyclopropyl-N-[4-[2-(3,3-difluoroazetidine-1-carbonyl)-4-fluorophenyl]-6-propan-2-yloxypyridin-2-yl]-5-formyl-2-oxopyridine-3-carboxamide and [(2S)-oxolan-2-yl]methanamine in step e. ¹H NMR (400 MHz, Chloroform-d) δ 12.21 (s, 1H), 8.56 (d, J=2.6 Hz, 1H), 8.03 (d, J=1.3 Hz, 1H), 7.66 (d, J=2.6 Hz, 1H), 7.57-7.49 (m, 1H), 7.24-7.16 (m, 2H), 6.52 (d, J=1.3 Hz, 1H), 5.50-5.39 (m, 1H), 4.35 (t, J=11.9 Hz, 2H), 4.07-3.92 (m, 3H), 3.91-3.72 (m, 2H), 3.68 (s, 2H), 3.53-3.43 (m, 1H), 2.81-2.55 (m, 2H), 2.07-1.84 (m, 3H), 1.58-1.54 (m, 1H), 1.33 (d, J=6.2 Hz, 6H), 1.30-1.22 (m, 2H), 1.02-0.94 (m, 2H). ESI MS [M+H]⁺ for C₃₃H₃₆F₃N₅O₅, calcd 640.2, found 640.2.

### Example 139: N-(4-{2-[(3,3-difluoro-1-azetidinyl)carbonyl]-4-fluorophenyl}-6-isopropoxy-2-pyridyl)-1-cyclopropyl-5-{[(cyclopropylmethyl)amino]methyl}-2-oxo-1,2-dihydronicotinamide

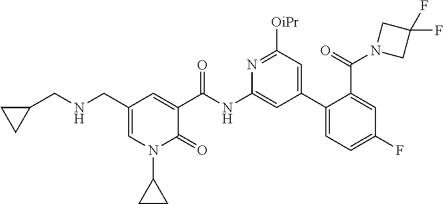 [SMILES: CC(C)Oc1cc(-c2ccc(F)cc2C(=O)N2CC(F)(F)C2)cc(NC(=O)c2cc(CNCC3CC3)cn(C3CC3)c2=O)n1]

The title compound was prepared in a similar fashion to that described for Example 137 using 1-cyclopropyl-N-[4-[2-(3,3-difluoroazetidine-1-carbonyl)-4-fluorophenyl]-6-propan-2-yloxypyridin-2-yl]-5-formyl-2-oxopyridine-3-carboxamide and cyclopropylmethanamine in step e. ¹H NMR (400 MHz, Chloroform-d) δ 12.21 (s, 1H), 8.56 (d, J=2.7 Hz, 1H), 8.03 (d, J=1.4 Hz, 1H), 7.65 (d, J=2.6 Hz, 1H), 7.58-7.48 (m, 1H), 7.25-7.15 (m, 2H), 6.52 (d, J=1.3 Hz, 1H), 5.56-5.39 (m, 1H), 4.35 (t, J=12.0 Hz, 2H), 3.99 (t, J=11.6 Hz, 2H), 3.67 (s, 2H), 3.54-3.40 (m, 1H), 2.50 (d, J=6.8 Hz, 2H), 1.34 (d, J=6.2 Hz, 6H), 1.30-1.25 (m, 3H), 1.00-0.95 (m, 2H), 0.55-0.47 (m, 2H), 0.15-0.10 (m, 2H). ESI MS [M+H]⁺ for C₃₂H₃₄F₃N₅O₄, calcd 640.2, found 640.2.

### Example 140: N-(4-{4-cyano-2-[(3,3-difluoro-1-azetidinyl)carbonyl]phenyl}-6-isopropoxy-2-pyridyl)-1-cyclopropyl-5-{[(S)-1-cyclopropylethylamino]methyl}-2-oxo-1,2-dihydronicotinamide

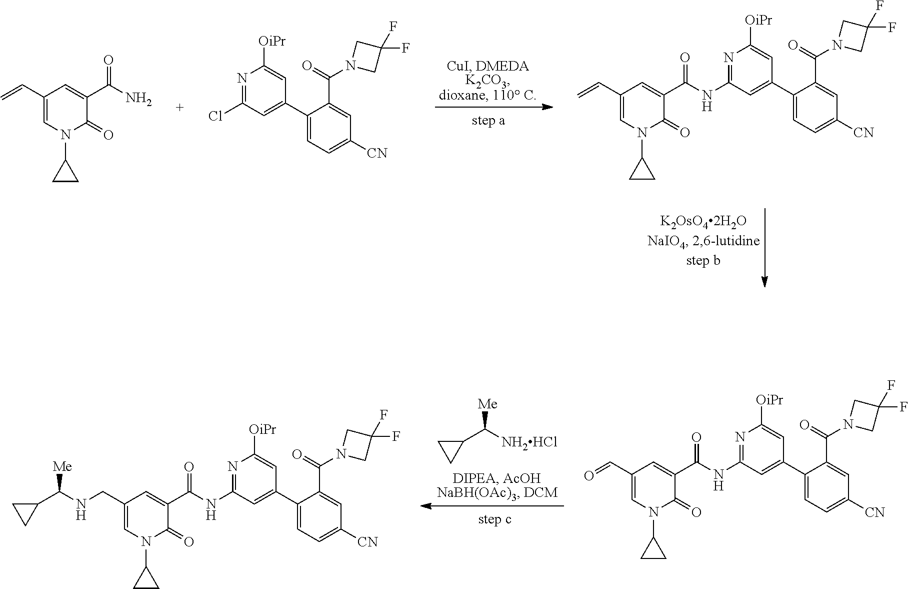

Step a: To a mixture of the 1-cyclopropyl-5-ethenyl-2-oxopyridine-3-carboxamide (332 mg, 1.63 mmol, 1.0 equiv.) and 4-(2-chloro-6-propan-2-yloxypyridin-4-yl)-3-(3,3-difluoroazetidine-1-carbonyl)benzonitrile (520 mg, 1.63 mmol, 1.0 equiv., prepared as described in example 96) in dioxane (10 mL) was added CuI (619 mg, 3.26 mmol, 2.0 equiv.), N, N′-dimethylethylenediamine (0.7 ml, 6.52 mmol, 4.0 equiv.) and K₂CO₃ (1.35 g, 9.78 mmol, 6.0 equiv.) The resulting mixture was heated at 110° C. overnight. The reaction mixture was cooled to room temperature, diluted with EtOAc, washed with H₂O and brine, filtered through Na₂SO₄, and concentrated. The crude product was purified by column chromatography (SiO₂, 0-100% EtOAc gradient in hexanes) to afford desired product.

Step b: To a solution of the product from step a (505 mg, 0.9 mmol, 1.0 equiv.) in a 1:1 mixture of THF/H₂O (10 mL) was added 2,6-lutidene (0.2 mL, 1.8 mmol, 2.0 equiv.) followed by NaIO₄ (770 mg, 3.6 mmol, 4.0 equiv.) and K₂OsO₄·2H₂O (33 mg, 0.09 mmol, 0.1 equiv.). The reaction mixture was stirred for 16 hours at room temperature at which point it was quenched with saturated aqueous Na₂S₂O₃ (100 mL) and extracted with EtOAc (2×100 mL). The combined organics were dried over Na₂SO₄, filtered, and concentrated under vacuum. The crude residue was used directly without further purification.

Step c: To a solution of the product of step b (56.1 mg, 0.1 mmol, 1.0 equiv.) in DCM (1.0 mL) was added (αR)-α-methylcyclopropanemethanamine (27.0 mg, 0.2 mmol, 2.0 equiv.) followed by sodium triacetoxyborohydride (64 mg, 0.3 mmol, 3.0 equiv.), DIPEA (70 μL, 0.4 mmol, 2.0 equiv.) and AcOH (12 μL, 0.2 mmol, 2.0 equiv.). The reaction mixture was stirred for 16 hours at room temperature at which point it was quenched with H₂O (2 mL) and extracted with DCM (2×2 mL). The combined organics were dried over Na₂SO₄, filtered, and concentrated under vacuum. The crude residue was purified by reverse phase prep-HPLC (C18 column, 5 to 60% MeCN/H₂O) to afford the desired product. ¹H NMR (400 MHz, Chloroform-d) δ 12.23 (s, 1H), 8.57 (d, J=2.7 Hz, 1H), 8.03 (d, J=1.4 Hz, 1H), 7.80 (d, J=1.4 Hz, 1H), 7.78 (d, J=1.7 Hz, 1H), 7.68-7.64 (m, 1H), 6.54 (d, J=1.4 Hz, 1H), 5.45 (p, J=6.1 Hz, 1H), 4.38 (t, J=11.9 Hz, 2H), 4.02 (t, J=11.4 Hz, 2H), 3.91-3.68 (m, 2H), 3.46 (tt, J=7.7, 4.3 Hz, 1H), 2.15-1.95 (m, 3H), 1.34 (d, J=6.1 Hz, 6H), 1.24 (t, J=6.3 Hz, 5H), 1.00 (dd, J=6.3, 3.9 Hz, 2H), 0.82 (dt, J=9.2, 4.8 Hz, 1H), 0.54 (dtt, J=27.0, 8.9, 4.4 Hz, 2H), 0.25 (dq, J=9.5, 4.8 Hz, 1H), 0.13 (dq, J=9.8, 4.7 Hz, 1H). ESI MS [M+H]⁺ for C₃₄H₃₇F₂N₆O₄ calcd. 631.3 found 631.3.

### Example 141: N-(4-{4-cyano-2-[(3,3-difluoro-1-azetidinyl)carbonyl]phenyl}-6-isopropoxy-2-pyridyl)-1-cyclopropyl-5-{[(S)-1-cyclopropylethylamino]methyl}-2-oxo-1,2-dihydronicotinamide

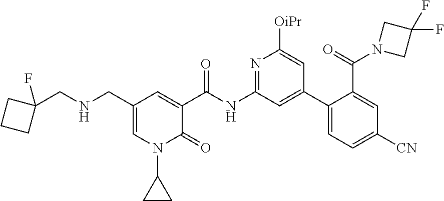 [SMILES: CC(C)Oc1cc(-c2ccc(C#N)cc2C(=O)N2CC(F)(F)C2)cc(NC(=O)c2cc(CNCC3(F)CCC3)cn(C3CC3)c2=O)n1]

The title compound was prepared in a similar fashion to that described for Example 140 using (1-fluorocyclobutyl)methanamine hydrochloride in step c. ¹H NMR (400 MHz, Chloroform-d) δ 12.26 (s, 1H), 8.58 (d, J=2.6 Hz, 1H), 8.05 (d, J=1.5 Hz, 1H), 7.79 (d, J=7.4 Hz, 2H), 7.66 (dd, J=5.8, 2.7 Hz, 2H), 6.54 (d, J=1.4 Hz, 1H), 5.46 (p, J=6.2 Hz, 1H), 4.38 (t, J=11.9 Hz, 2H), 4.03 (t, J=11.6 Hz, 2H), 3.72 (s, 2H), 3.59-3.35 (m, 1H), 2.89 (s, 1H), 2.83 (s, 1H), 2.31 (dq, J=21.0, 10.5 Hz, 2H), 2.18 (t, J=9.3 Hz, 2H), 1.90-1.75 (m, 2H)1.46 (q, J=9.2 Hz, 1H), 1.34 (d, J=6.1 Hz, 6H), 1.27 (q, J=6.8 Hz, 2H), 0.98 (t, J=5.6 Hz, 2H). ESI MS [M+H]+ for C₃₄H₃₆F₃N₆O₄ calcd. 649.3 found 649.3.

### Example 142: N-(4-{4-cyano-2-[(3,3-difluoro-1-azetidinyl)carbonyl]phenyl}-6-isopropoxy-2-pyridyl)-1-cyclopropyl-5-({[(1-fluorocyclobutyl)methyl]amino}methyl)-2-oxo-1,2-dihydronicotinamide

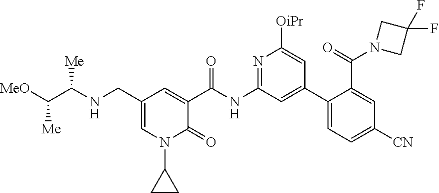 [SMILES: CO[C@@H](C)[C@H](C)NCc1cc(C(=O)Nc2cc(-c3ccc(C#N)cc3C(=O)N3CC(F)(F)C3)cc(OC(C)C)n2)c(=O)n(C2CC2)c1]

The title compound was prepared in a similar fashion to that described for Example 140 using (2S,3S)-3-methoxybutan-2-amine in step c. ¹H NMR (400 MHz, Chloroform-d) δ 12.27 (s, 1H), 8.57 (d, J=2.7 Hz, 1H), 8.06 (d, J=1.3 Hz, 1H), 7.82-7.77 (m, 2H), 7.72-7.62 (m, 2H), 6.53 (d, J=1.4 Hz, 1H), 5.46 (p, J=6.2 Hz, 1H), 4.38 (t, J=11.8 Hz, 2H), 4.03 (t, J=11.5 Hz, 2H), 3.74 (d, J=13.5 Hz, 1H), 3.57 (d, J=13.6 Hz, 1H), 3.47 (tt, J=7.7, 4.1 Hz, 1H), 3.35 (s, 3H), 3.16 (dd, J=13.0, 6.5 Hz, 1H), 2.62 (p, J=6.6 Hz, 1H), 1.84-1.53 (m, 1H), 1.34 (d, J=6.2 Hz, 6H), 1.26 (dd, J=7.3, 3.7 Hz, 2H), 1.14 (d, J=6.1 Hz, 3H), 1.06 (d, J=6.4 Hz, 3H), 1.02-0.93 (m, 2H). ESI MS [M+H]⁺ for C₃₄H₃₉F₂N₆O₅ calcd. 649.3 found 649.3.

### Example 143: N-(4-{4-cyano-2-[(3,3-difluoro-1-azetidinyl)carbonyl]phenyl}-6-isopropoxy-2-pyridyl)-5-{[(S)-1-cyclobutylethylamino]methyl}-1-cyclopropyl-2-oxo-1,2-dihydronicotinamide

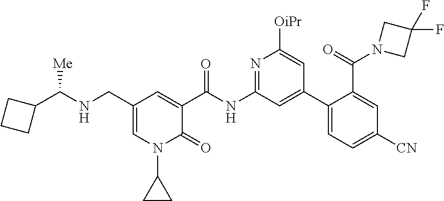 [SMILES: CC(C)Oc1cc(-c2ccc(C#N)cc2C(=O)N2CC(F)(F)C2)cc(NC(=O)c2cc(CN[C@@H](C)C3CCC3)cn(C3CC3)c2=O)n1]

The title compound was prepared in a similar fashion to that described for Example 140 using (1S)-1-cyclobutylethan-1-amine hydrochloride in step c. ¹H NMR (400 MHz, Chloroform-d) δ 12.25 (s, 1H), 8.55 (d, J=2.6 Hz, 1H), 8.04 (d, J=1.4 Hz, 1H), 7.84-7.77 (m, 2H), 7.71 (d, J=2.7 Hz, 1H), 7.69-7.63 (m, 1H), 6.53 (d, J=1.4 Hz, 1H), 5.45 (p, J=6.2 Hz, 1H), 4.38 (t, J=11.9 Hz, 2H), 4.03 (t, J=11.5 Hz, 2H), 3.73 (d, J=13.5 Hz, 1H), 3.61 (d, J=13.5 Hz, 1H), 3.47 (tt, J=7.6, 4.3 Hz, 1H), 2.70-2.54 (m, 1H), 2.27 (q, J=8.3 Hz, 1H), 2.09-1.82 (m, 5H), 1.72 (dq, J=17.1, 8.5 Hz, 3H), 1.34 (d, J=6.1 Hz, 6H), 1.31-1.22 (m, 2H), 0.99 (dd, J=10.3, 5.3 Hz, 5H). ESI MS [M+H]+ for C₃₅H₃₉F₂N₆O₄ calcd. 645.3 found 645.3.

### Example 144: N-(4-{4-chloro-2-[(3,3-difluoro-1-azetidinyl)carbonyl]-6-fluorophenyl}-6-cyclopropyl-2-pyridyl)-1-cyclopropyl-5-[(isobutylamino)methyl]-2-oxo-1,2-dihydronicotinamide

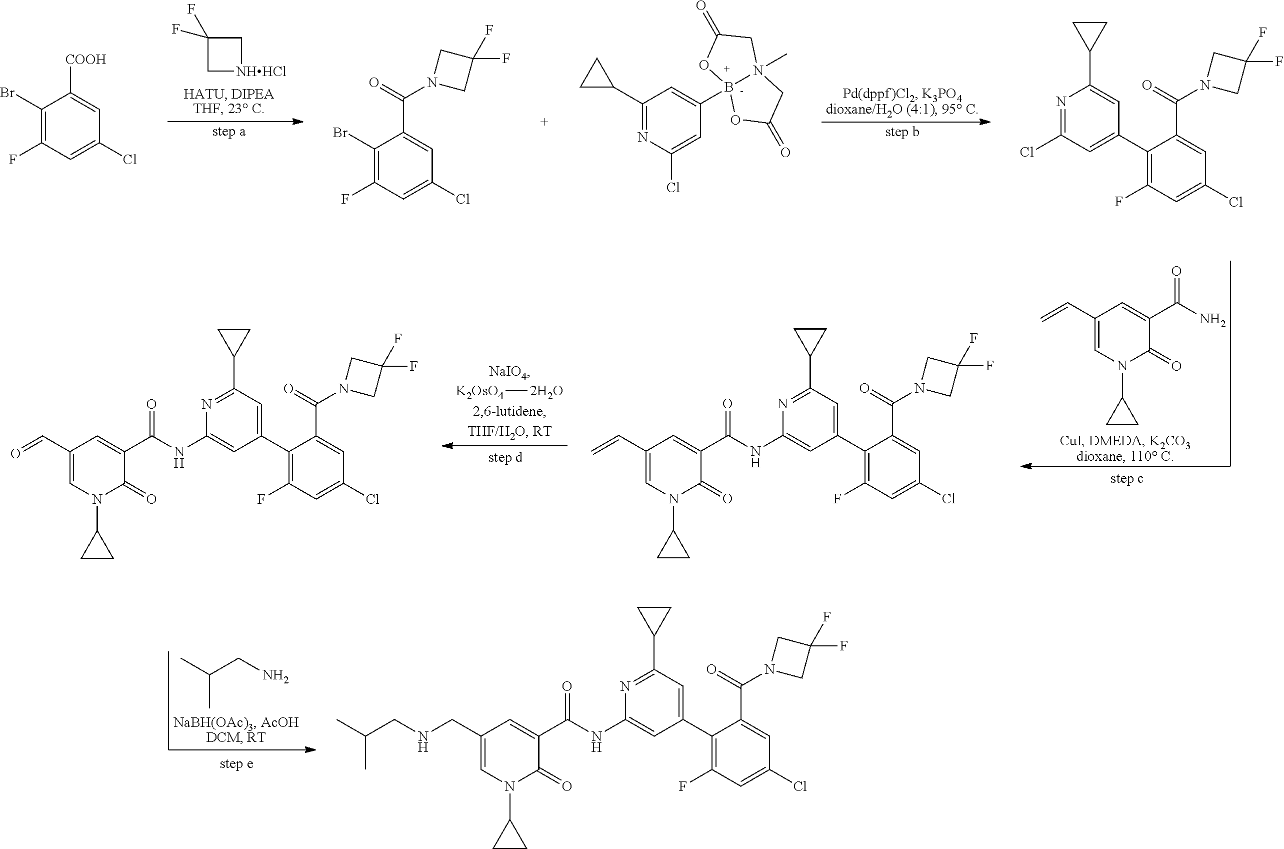

Step a: To a solution of 2-bromo-5-chloro-3-fluorobenzoic acid (0.76 g, 3.0 mmol, 1 equiv.) and 3,3-difluoroazetidine hydrochloride (0.50 g, 3.9 mmol, 1.3 equiv.) in THF (15 mL, 0.2 M) was added DIPEA (1.06 mL, 6.0 mmol, 2 equiv) followed by HATU (1.7 g, 4.5 mmol, 1.5 equiv.). The reaction mixture was stirred for 16 hours at room temperature, at which point it was partitioned between EtOAc (50 mL) and water (10 mL). The aqueous layer was extracted with EtOAc (2×25 mL), and the combined organics were dried over Na₂SO₄, filtered, and concentrated under a vacuum. The crude residue was purified by column chromatography (SiO₂, 0-100% EtOAc in hexanes) to afford the desired product.

Step b: A 40 mL vial was charged with the product from step a (0.32 g, 1.0 mmol. 1 equiv.), 1-(2-chloro-6-cyclopropylpyridin-4-yl)-5-methyl-2,8-dioxa-5-azonia-1-boranuidabicyclo[3.3.0]octane-3,7-dione (0.33 g, 1.1 mmol, 1.1 equiv., prepared according to example 15), and K₃PO₄ (0.63 g, 6.0 mmol, 3 equiv.). The reagents were suspended in a mixture of dioxane/water (10 mL, 4:1 v/v, 0.1 M), and the resulting solution was sparged with N₂ for 10 minutes. Then Pd(dppf)Cl₂ (73.1 mg, 0.1 mmol, 0.1 equiv) was added, and the mixture was heated to 95° C. for 4 hours. The reaction mixture was partitioned between EtOAc (75 mL) and water (25 mL). The organic phase was separated, and the aqueous phase was additionally extracted with EtOAc (3×50 mL). The combined organics were dried over Na₂SO₄ and concentrated to dryness under reduced pressure. The crude residue was purified via column chromatography (SiO₂, 0-80% EtOAc gradient in hexane) to afford the desired product.

Step c: To a suspension of the product from step b (0.32 g, 0.82 mmol, 1.0 equiv.), 1-cyclopropyl-5-ethenyl-2-oxopyridine-3-carboxamide (0.17 g, 0.82 mmol, 1.0 equiv.), and K₂CO₃ (0.34 g, 2.46 mmol, 3.0 equiv.) in dioxane (16 mL) was added CuI (0.15 g, 0.82 mmol, 1.0 equiv.) and DMEDA (0.17 mL, 1.64 mmol, 2.0 equiv.). The reaction mixture was heated to 110° C. and stirred for 16 hours, at which point it was quenched with a 1:1 mixture of water/brine (20 mL) and extracted with EtOAc (2×75 mL). The combined organics were dried over Na₂SO₄, filtered, and concentrated under a vacuum. The crude residue was purified by column chromatography (SiO₂, EtOAc in hexanes, 0 to 100%) to afford the desired product.

Step d: To a solution of the product from step c (0.24 g, 0.43 mmol, 1.0 equiv.) in a 1:1 mixture of THF/H₂O (3 mL) was added 2,6-lutidene (0.1 mL, 0.86 mmol, 2.0 equiv.) followed by NaIO₄ (0.37 g, 1.74 mmol, 4.0 equiv.) and K₂OsO₄*2H₂O (15.8 mg, 0.04 mmol, 0.1 equiv.). The reaction mixture was stirred for 4 hours at room temperature, at which point it was quenched with saturated aqueous Na₂S₂O₃ (20 mL) and extracted with EtOAc (2×50 mL). The combined organics were dried over Na₂SO₄, filtered, and concentrated under a vacuum. The crude residue was purified by column chromatography (SiO₂, EtOAc in hexanes, 0 to 100%) to afford the desired product.

Step e: To a solution of the product from step d (27.6 mg, 0.05 mmol, 1.0 equiv.) in DCM (1.0 mL) was added isobutylamine (7.3 mg, 0.1 mmol, 2.0 equiv.) followed by sodium triacetoxyborohydride (30 mg, 0.13 mmol, 2.5 equiv.) and AcOH (8 L, 0.13 mmol, 2.5 equiv.). The reaction mixture was stirred for 16 hours at room temperature, at which point it was quenched with saturated aqueous NaHCO₃ (10 mL) and extracted with EtOAc (2×25 mL). The combined organics were dried over Na₂SO₄, filtered, and concentrated under a vacuum. The crude residue was purified by reverse phase prep-HPLC (C18 column, 5 to 60% MeCN/H₂O) to afford the desired product. ¹H NMR (400 MHz, Chloroform-d) δ 12.26 (s, 1H), 8.53 (d, J=2.6 Hz, 1H), 8.18 (t, J=1.4 Hz, 1H), 7.71 (d, J=2.6 Hz, 1H), 7.34-7.27 (m, 2H), 6.95 (t, J=1.4 Hz, 1H), 4.28 (t, J=11.9 Hz, 2H), 4.11 (t, J=11.5 Hz, 2H), 3.69 (s, 2H), 3.49-3.40 (m, 1H), 2.47 (d, J=6.8 Hz, 2H), 2.00-1.96 (m, 1H), 1.85-1.74 (m, 1H), 1.29-1.21 (m, 2H), 1.15-1.04 (m, 2H), 1.03-0.83 (m, 10H). ESI MS [M+H]⁺ for C₃₂H₃₃ClF₃N₅O₃, calcd 628.2, found 628.2.

### Example 145: N-(4-{4-chloro-2-[(3,3-difluoro-1-azetidinyl)carbonyl]-6-fluorophenyl}-6-cyclopropyl-2-pyridyl)-1-cyclopropyl-2-oxo-5-[({[(S)-perhydro-2-furyl]methyl}amino)methyl]-1,2-dihydronicotinamide

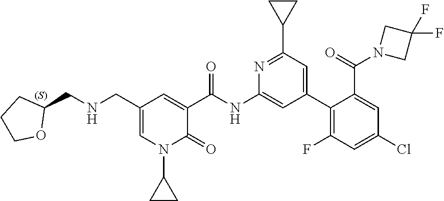

The title compound was prepared in a similar fashion to that described for Example 144 using N-[4-[4-chloro-2-(3,3-difluoroazetidine-1-carbonyl)-6-fluorophenyl]-6-cyclopropylpyridin-2-yl]-1-cyclopropyl-5-formyl-2-oxopyridine-3-carboxamide and [(2S)-oxolan-2-yl]methanamine in step e. ¹H NMR (400 MHz, Chloroform-d) δ 12.26 (s, 1H), 8.53 (d, J=2.6 Hz, 1H), 8.18 (t, J=1.5 Hz, 1H), 7.71 (d, J=2.6 Hz, 1H), 7.32-7.27 (m, 2H), 6.95 (t, J=1.4 Hz, 1H), 4.28 (t, J=11.8 Hz, 2H), 4.11 (t, J=11.5 Hz, 2H), 4.06-3.98 (m, 1H), 3.89-3.82 (m, 1H), 3.79-3.69 (m, 3H), 3.50-3.41 (m, 1H), 2.80-2.74 (m, 1H), 2.69-2.59 (m, 1H), 1.99-1.85 (m, 5H), 1.62-1.50 (m, 1H), 1.29-1.21 (m, 2H), 1.14-1.06 (m, 2H), 1.01-0.92 (m, 4H). ESI MS [M+H]⁺ for C₃₃H₃₃ClF₃N₅O₄, calcd 656.2, found 656.2.

### Example 146: N-(4-{4-chloro-2-[(3,3-difluoro-1-azetidinyl)carbonyl]-6-fluorophenyl}-6-cyclopropyl-2-pyridyl)-1-cyclopropyl-2-oxo-5-[({[(S)-tetrahydro-2H-pyran-2-yl]methyl}amino)methyl]-1,2-dihydronicotinamide

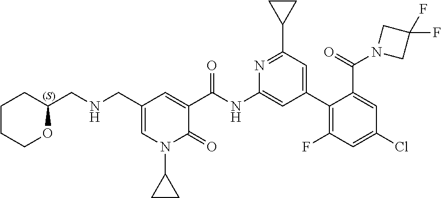

The title compound was prepared in a similar fashion to that described for Example 144 using N-[4-[4-chloro-2-(3,3-difluoroazetidine-1-carbonyl)-6-fluorophenyl]-6-cyclopropylpyridin-2-yl]-1-cyclopropyl-5-formyl-2-oxopyridine-3-carboxamide and [(2S)-oxan-2-yl]methanamine in step e. ¹H NMR (400 MHz, Chloroform-d) δ 12.25 (s, 1H), 8.53 (d, J=2.6 Hz, 1H), 8.24-8.11 (m, 1H), 7.81 (s, 1H), 7.29 (d, J=8.0 Hz, 2H), 6.95 (t, J=1.3 Hz, 1H), 4.28 (t, J=11.9 Hz, 2H), 4.10 (t, J=11.5 Hz, 2H), 3.99-3.92 (m, 1H), 3.74 (s, 2H), 3.56-3.37 (m, 3H), 2.77-2.60 (m, 2H), 2.01-1.97 (m, 1H), 1.89-1.80 (m, 1H), 1.58-1.50 (m, 3H), 1.37-1.20 (m, 4H), 1.13-1.07 (m, 2H), 1.04-0.92 (m, 4H). ESI MS [M+H]⁺ for C₃₄H₃₅ClF₃N₅O₄, calcd 670.2, found 670.2.

### Example 147: N-(4-{4-chloro-2-[(3,3-difluoro-1-azetidinyl)carbonyl]-6-fluorophenyl}-6-cyclopropyl-2-pyridyl)-1-cyclopropyl-5-[(2-methoxyethylamino)methyl]-2-oxo-1,2-dihydronicotinamide

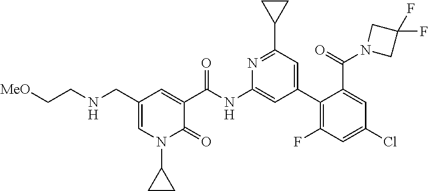 [SMILES: COCCNCc1cc(C(=O)Nc2cc(-c3c(F)cc(Cl)cc3C(=O)N3CC(F)(F)C3)cc(C3CC3)n2)c(=O)n(C2CC2)c1]

The title compound was prepared in a similar fashion to that described for Example 144 using N-[4-[4-chloro-2-(3,3-difluoroazetidine-1-carbonyl)-6-fluorophenyl]-6-cyclopropylpyridin-2-yl]-1-cyclopropyl-5-formyl-2-oxopyridine-3-carboxamide and 2-methoxyethanamine in step e. ¹H NMR (400 MHz, Chloroform-d) δ 12.25 (s, 1H), 8.54 (d, J=2.6 Hz, 1H), 8.18 (d, J=1.5 Hz, 1H), 7.70 (d, J=2.6 Hz, 1H), 7.33-7.27 (m, 2H), 6.95 (d, J=1.4 Hz, 1H), 4.28 (t, J=11.9 Hz, 2H), 4.11 (t, J=11.5 Hz, 2H), 3.70 (s, 2H), 3.56-3.50 (m, 2H), 3.48-3.41 (m, 1H), 3.36 (s, 3H), 2.84 (t, J=5.0 Hz, 2H), 2.03-1.95 (m, 1H), 1.24 (q, J=6.8 Hz, 2H), 1.14-1.07 (m, 2H), 1.00-0.92 (m, 4H). ESI MS [M+H]⁺ for C₃₁H₃₁ClF₃N₅O₄, calcd 630.2, found 630.2.

### Example 148: N-(4-{4-chloro-2-[(3,3-difluoro-1-azetidinyl)carbonyl]-6-fluorophenyl}-6-cyclopropyl-2-pyridyl)-1-cyclopropyl-5-{[(cyclopropylmethyl)amino]methyl}-2-oxo-1,2-dihydronicotinamide

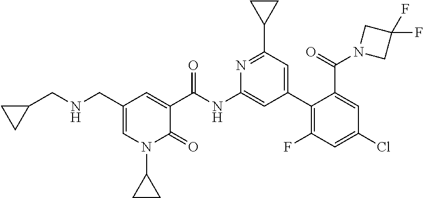 [SMILES: O=C(Nc1cc(-c2c(F)cc(Cl)cc2C(=O)N2CC(F)(F)C2)cc(C2CC2)n1)c1cc(CNCC2CC2)cn(C2CC2)c1=O]

The title compound was prepared in a similar fashion to that described for Example 144 using N-[4-[4-chloro-2-(3,3-difluoroazetidine-1-carbonyl)-6-fluorophenyl]-6-cyclopropylpyridin-2-yl]-1-cyclopropyl-5-formyl-2-oxopyridine-3-carboxamide and cyclopropylmethanamine in step e. ¹H NMR (400 MHz, Chloroform-d) δ 12.24 (s, 1H), 8.53 (d, J=2.7 Hz, 1H), 8.17 (t, J=1.5 Hz, 1H), 7.71 (d, J=2.6 Hz, 1H), 7.34-7.27 (m, 2H), 6.95 (t, J=1.4 Hz, 1H), 4.27 (t, J=11.9 Hz, 2H), 4.10 (t, J=11.5 Hz, 2H), 3.72 (s, 2H), 3.50-3.41 (m, 1H), 2.54 (d, J=7.0 Hz, 2H), 2.40-2.39 (m, 2H), 2.04-1.94 (m, 1H), 1.30-1.21 (m, 2H), 1.14-1.07 (m, 2H), 1.00-0.92 (m, 4H), 0.56-0.48 (m, 2H), 0.18-0.12 (m, 2H). ESI MS [M+H]⁺ for C₃₂H₃₁ClF₃N₅O₃, calcd 626.2, found 626.2.

### Example 149: N-(6-chloro-4-{2-[(3,3-difluoro-1-azetidinyl)carbonyl]-4-difluoromethoxyphenyl}-2-pyridyl)-1-cyclopropyl-5-{[(S)-2-methoxypropylamino]methyl}-2-oxo-1,2-dihydronicotinamide

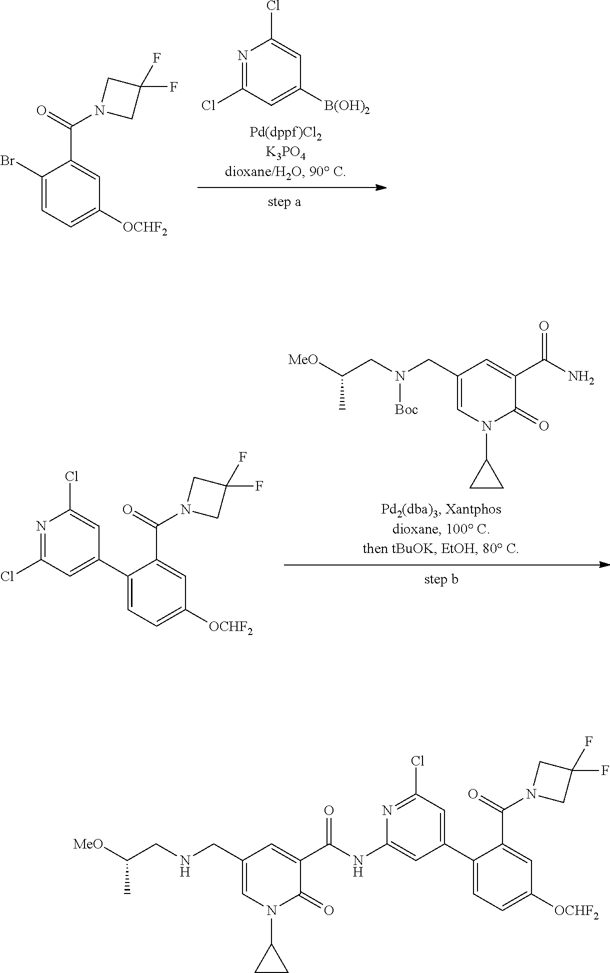

Step a: To a solution of [2-bromo-5-(difluoromethoxy)phenyl]-(3,3-difluoroazetidin-1-yl)methanone (260 mg, 0.7602 mmol, 1.0 equiv.) (prepared according to Example 98), (2,6-dichloropyridin-4-yl)boronic acid (161 mg, 0.8363 mmol. 1.1 equiv.) and K₃PO₄ (484 mg, 2.2806 mmol, 3.0 equiv.) in dioxane (10 mL, 0.05 M) and H₂O (2 mL, 0.05 M) was added Pd(dppf)Cl₂ (56 mg, 0.07602 mmol, 0.1 equiv.) at rt under nitrogen atmosphere. The resulting mixture was stirred for 2 h at 90° C. The mixture was allowed to cool down to rt. The reaction was quenched with sat. aq. NH₄Cl solution, the organic phase was separated, and the aqueous layer was extracted with EtOAc. The combined organic phase was dried over Na₂SO₄, concentrated and the crude residue was purified by column chromatography (SiO₂, hexane in EtOAc, 0 to 60%) to afford [2-(2,6-dichloropyridin-4-yl)-5-(difluoromethoxy)phenyl]-(3,3-difluoroazetidin-1-yl)methanone.

Step b: A solution of the product from step a (136 mg, 0.33 mmol, 1.0 equiv.), tert-butyl N-[(5-carbamoyl-1-cyclopropyl-6-oxopyridin-3-yl)methyl]-N-[(2S)-2-methoxypropyl]carbamate (127 mg, 0.33 mmol, 1.0 equiv., prepared according to example 89), K₂CO₃ (140 mg, 0.99 mmol, 3.0 equiv.) and Xantphos (13 mg, 0.033 mmol, 0.1 equiv.) in dioxane (5 ml, 0.06 M) was degassed with a stream of bubbling nitrogen for ten minutes. Then, Pd₂(dba)₃ (15 mg, 0.017 mmol, 0.05 equiv.) were added, and the reaction was heated for 16 h at 100° C. Then the reaction mixture was cooled to room temperature, diluted with aq. NH₄Cl (2 mL) and extracted with EtOAc (2×5 mL). The combined organic phase was washed with water (2×4 mL) and brine (4 mL), dried over Na₂SO₄, and concentrated to dryness. The crude residue was purified by reversed-phase column chromatography (C18, 10-100% CH₃CN in water with 0.1% formic acid) to afford the corresponding coupling product. The residual material was then treated with tBuOK (30 mg, 0.266 mmol, 2.0 equiv.) in EtOH (5 ml, 0.01 M) at 80° C. for 6 h. The mixture was concentrated under vacuum, and the crude residue was then purified by reversed-phase column chromatography (C18, 10-100% CH₃CN in water with 0.1% formic acid) to afford the title compound. ¹H NMR (400 MHz, Chloroform-d) δ 12.67 (s, 1H), 8.53 (d, J=2.7 Hz, 1H), 8.40 (d, J=1.4 Hz, 1H), 7.65 (d, J=2.6 Hz, 1H), 7.57-7.49 (m, 1H), 7.30 (dd, J=10.7, 2.4 Hz, 1H), 7.13 (d, J=1.3 Hz, 1H), 7.01-6.36 (m, 1H), 4.40 (t, J=11.9 Hz, 2H), 4.06 (t, J=11.5 Hz, 2H), 3.65 (d, J=2.6 Hz, 2H), 3.53-3.40 (m, 2H), 3.36 (s, 3H), 2.70-2.55 (m, 2H), 1.31-1.21 (m, 3H), 1.15 (d, J=6.1 Hz, 3H), 1.05-0.92 (m, 2H). ESI MS [M+H]⁺ for C₃₀H₃₁F₄N₅O₅, calcd 653.1, found 653.1.

### Example 150: N-(6-Cyclopropyl-4-{2-[(3,3-difluoro-1-azetidinyl)carbonyl]-4-methoxyphenyl}-2-pyridyl)-1-cyclopropyl-5-{[(S)-2-methoxypropylamino]methyl}-2-oxo-1,2-dihydronicotinamide

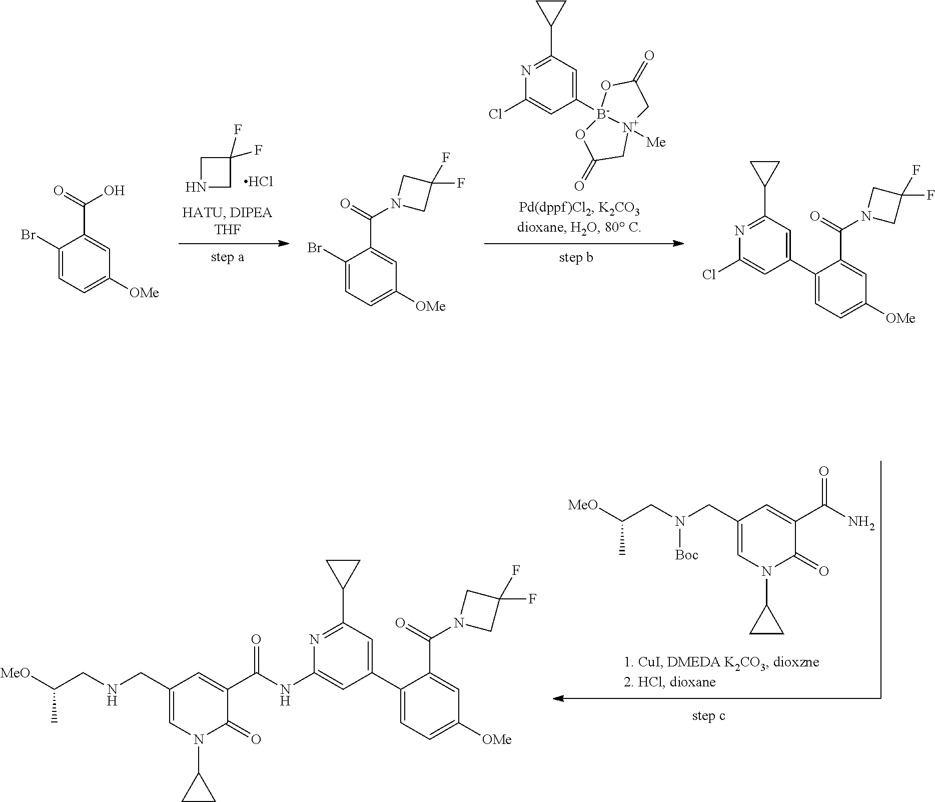

Step a: To a solution of 2-bromo-5-methoxybenzoic acid (1.00 g, 4.3 mmol, 1.0 equiv.) and 3,3-difluoroazetidine hydrochloride (674 mg, 5.2 mmol, 1.2 equiv.) in THF (20 mL) was added HATU (1.98 g, 5.2 mmol, 1.2 equiv.) and DIPEA (2.2 mL, 1.67 g, 13 mmol, 3.0 equiv.) at room temperature. The resulting mixture was stirred at room temperature for overnight and then concentrated. The residue was purified by column chromatography (SiO₂, EtOAc in hexanes, 20 to 65%) to give the product.

Step b: To a mixture of the product from step a (612 mg, 2.0 mmol, 1.0 equiv.) and 1-(2-chloro-6-cyclopropylpyridin-4-yl)-5-methyl-2,8-dioxa-5-azonia-1-boranuidabicyclo[3.3.0]octane-3,7-dione (617 mg, 2.0 mmol, 1.0 equiv., prepared according to example 15) in dioxane (5.0 mL) was added Pd(dppf)Cl₂ (146 mg, 0.20 mmol, 10 mol %) and 1M K₂CO₃ aqueous solution (4.0 mL, 4.0 mmol, 2.0 equiv.). The resulting mixture was heated at 80° C. for 2 h and cooled to room temperature. The organic phase was separated, and the aqueous layer was extracted with EtOAc (2×25 mL). The combined organic phase was dried over Na₂SO₄, concentrated, and the crude residue was purified by column chromatography (SiO₂, EtOAc in hexanes, 0 to 50%) to give the product.

Step c: To a solution of the product from step b (56.8 mg, 0.15 mmol, 1.0 equiv.) and tert-butyl N-[(5-carbamoyl-1-cyclopropyl-6-oxopyridin-3-yl)methyl]-N-[(2S)-2-methoxypropyl]carbamate (the product of step b in Example 102, 56.9 mg, 0.15 mmol, 1.0 equiv.) in dioxane (1.0 mL) was added CuI (28.6 mg, 0.15 mmol, 1.0 equiv.), DMEDA (26.4 mg, 0.30 mmol, 2.0 equiv.) and K₂CO₃ (66.2 mg, 0.45 mmol, 3.0 equiv.). The resulting mixture was heated at 110° C. under N₂ for overnight. After cooled to room temperature, 4M HCl in dioxane (1 mL) was added. The reaction mixture was then stirred at room temperature for another 1 h before diluted with EtOAc and H₂O. The mixture was then filtered through Celite® to remove the precipitates. The filtrate was neutralized with saturated K₂CO₃ aqueous solution and separated. The aqueous layer was then extracted with EtOAc (2×25 mL). The combined organic phase was washed with brine, dried over Na₂SO₄, concentrated, and the crude residue was purified by HPLC to afford the title compound. ¹H NMR (400 MHz, DMSO-d₆) δ 12.44 (s, 1H), 8.47 (d, J=2.6 Hz, 1H), 8.07 (d, J=1.5 Hz, 1H), 7.92 (d, J=2.6 Hz, 1H), 7.52 (d, J=8.5 Hz, 1H), 7.17 (dd, J=8.6, 2.7 Hz, 1H), 7.11 (d, J=2.7 Hz, 1H), 7.01 (d, J=1.5 Hz, 1H), 4.33 (dt, J=23.0, 12.2 Hz, 4H), 3.86 (s, 3H), 3.58 (s, 2H), 3.50 (tt, J=7.6, 4.3 Hz, 1H), 3.37 (td, J=6.3, 4.9 Hz, 1H), 3.23 (s, 3H), 2.57-2.52 (m, 1H), 2.48-2.44 (m, 1H), 2.13-2.05 (m, 1H), 1.14-1.08 (m, 2H), 1.06 (d, J=6.2 Hz, 3H), 1.03-0.89 (m, 6H). ESI MS [M+H]⁺ for C₃₃H₃₇F₂N₅O₅, calcd 622.3, found 622.3.

### Example 151: N-(6-Cyclopropyl-4-{2-[(3,3-difluoro-1-azetidinyl)carbonyl]-4-methoxyphenyl}-2-pyridyl)-1-cyclopropyl-5-[(isobutylamino)methyl]-2-oxo-1,2-dihydronicotinamide

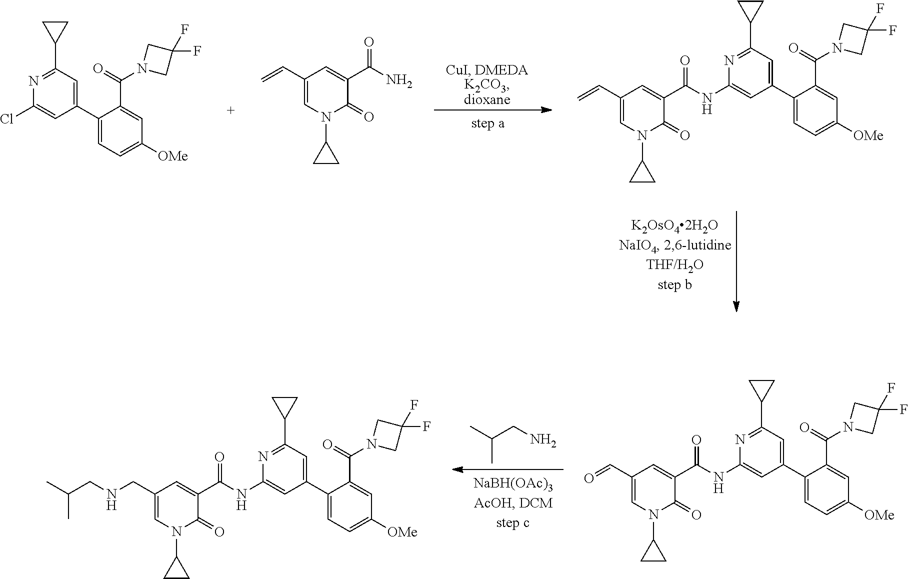

Step a: The reaction was performed in a similar fashion to Example 90, step c using [2-(2-chloro-6-cyclopropylpyridin-4-yl)-5-methoxyphenyl]-(3,3-difluoroazetidin-1-yl)methanone and 1-cyclopropyl-5-ethenyl-2-oxopyridine-3-carboxamide.

Step b: The reaction was performed in a similar fashion to Example 90, step d.

Step c: To a solution of the crude product from step b (˜0.10 mmol) in DCM (1.0 mL) was added 2-methylpropan-1-amine (14.6 mg, 0.20 mmol, 2.0 equiv.) and AcOH (12.0 mg, 0.20 mmol, 2.0 equiv.). The resulting mixture was stirred at room temperature for 30 min before NaBH(OAc)₃ (42.4 mg, 0.20 mmol, 2.0 equiv.) was added. The reaction mixture was then stirred at room temperature for overnight. The mixture was then diluted with EtOAc, washed with water and brine, dried over Na₂SO₄, and concentrated. The crude residue was purified by HPLC to afford the title compound. ¹H NMR (400 MHz, DMSO-d₆) δ 12.45 (s, 1H), 8.48 (d, J=2.6 Hz, 1H), 8.07 (d, J=1.5 Hz, 1H), 7.90 (d, J=2.6 Hz, 1H), 7.52 (d, J=8.6 Hz, 1H), 7.16 (dd, J=8.6, 2.7 Hz, 1H), 7.11 (d, J=2.7 Hz, 1H), 7.01 (d, J=1.5 Hz, 1H), 4.33 (dt, J=22.1, 12.3 Hz, 4H), 3.86 (s, 3H), 3.55 (s, 2H), 3.50 (tt, J=7.6, 4.3 Hz, 1H), 2.29 (d, J=6.7 Hz, 2H), 2.09 (tt, J=8.0, 4.9 Hz, 1H), 1.66 (hept, J=6.7 Hz, 1H), 1.17-1.07 (m, 2H), 1.01-0.90 (m, 6H), 0.86 (d, J=6.6 Hz, 6H). ESI MS [M+H]⁺ for C₃₃H₃₇F₂N₅O₄, calcd 606.3, found 606.3.

### Example 152: N-(6-Cyclopropyl-4-{2-[(3,3-difluoro-1-azetidinyl)carbonyl]-4-methoxyphenyl}-2-pyridyl)-1-cyclopropyl-5-{[(1S,2S)-2-methoxy-1-methylpropylamino]methyl}-2-oxo-1,2-dihydronicotinamide

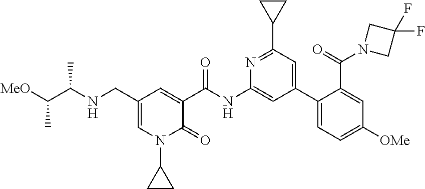 [SMILES: COc1ccc(-c2cc(NC(=O)c3cc(CN[C@@H](C)[C@H](C)OC)cn(C4CC4)c3=O)nc(C3CC3)c2)c(C(=O)N2CC(F)(F)C2)c1]

The title compound was prepared in a similar fashion to that described for Example 151 with (2S,3S)-3-methoxybutan-2-amine hydrochloride in step c. ¹H NMR (400 MHz, DMSO-d₆) δ 12.45 (s, 1H), 8.47 (d, J=2.6 Hz, 1H), 8.07 (d, J=1.5 Hz, 1H), 7.92 (d, J=2.6 Hz, 1H), 7.52 (d, J=8.6 Hz, 1H), 7.16 (dd, J=8.6, 2.7 Hz, 1H), 7.11 (d, J=2.7 Hz, 1H), 7.01 (d, J=1.5 Hz, 1H), 4.33 (dt, J=21.4, 12.2 Hz, 4H), 3.86 (s, 3H), 3.63 (d, J=13.7 Hz, 1H), 3.55-3.46 (m, 2H), 3.26-3.16 (m, 4H), 2.61 (p, J=6.4 Hz, 1H), 2.09 (ddd, J=12.8, 8.1, 4.9 Hz, 1H), 1.14-1.07 (m, 2H), 1.02 (d, J=6.3 Hz, 3H), 1.02-0.91 (m, 6H), 0.92 (d, J=6.4 Hz, 3H). ESI MS [M+H]⁺ for C₃₄H₃₉F₂N₅O₅, calcd 636.3, found 636.3.

### Example 153: N-(6-Cyclopropyl-4-{2-[(3,3-difluoro-1-azetidinyl)carbonyl]-4-methoxyphenyl}-2-pyridyl)-1-cyclopropyl-2-oxo-5-[({[(S)-perhydro-2-furyl]methyl}amino)methyl]-1,2-dihydronicotinamide

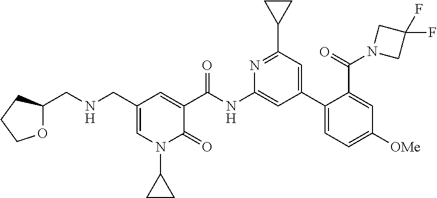 [SMILES: COc1ccc(-c2cc(NC(=O)c3cc(CNC[C@@H]4CCCO4)cn(C4CC4)c3=O)nc(C3CC3)c2)c(C(=O)N2CC(F)(F)C2)c1]

The title compound was prepared in a similar fashion to that described for example 151 with [(S)-oxolan-2-yl]methanamine in step c. ¹H NMR (400 MHz, DMSO-d₆) δ 12.45 (s, 1H), 8.46 (d, J=2.6 Hz, 1H), 8.07 (d, J=1.5 Hz, 1H), 7.90 (d, J=2.6 Hz, 1H), 7.52 (d, J=8.6 Hz, 1H), 7.17 (dd, J=8.6, 2.7 Hz, 1H), 7.11 (d, J=2.6 Hz, 1H), 7.01 (d, J=1.5 Hz, 1H), 4.33 (dt, J=23.3, 12.3 Hz, 4H), 3.93-3.80 (m, 4H), 3.72 (dt, J=8.0, 6.5 Hz, 1H), 3.64-3.54 (m, 3H), 3.50 (tt, J=7.7, 4.3 Hz, 1H), 2.57-2.52 (m, 2H), 2.09 (ddd, J=11.2, 8.1, 4.8 Hz, 1H), 1.94-1.84 (m, 1H), 1.83-1.71 (m, 2H), 1.59-1.45 (m, 1H), 1.15-1.06 (m, 2H), 1.04-0.86 (m, 6H). ESI MS [M+H]⁺ for C₃₄H₃₇F₂N₅O₅, calcd 634.3, found 634.3.

### Example 154: N-[4-[4-cyano-2-(3,3-difluoroazetidine-1-carbonyl)phenyl]-6-cyclopropylpyridin-2-yl]-5-[[[(1S,2S)-2-methoxycyclopentyl]amino]methyl]-2-oxo-1-(2,2,2-trifluoroethyl)pyridine-3-carboxamide

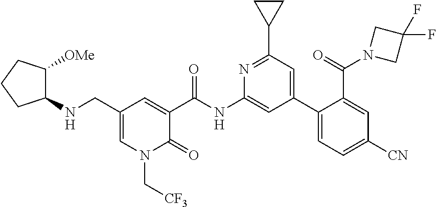 [SMILES: CO[C@H]1CCC[C@@H]1NCc1cc(C(=O)Nc2cc(-c3ccc(C#N)cc3C(=O)N3CC(F)(F)C3)cc(C3CC3)n2)c(=O)n(CC(F)(F)F)c1]

The title compound was prepared in a similar fashion to that described for Example 93 using (1S,2S)-2-methoxycyclopentan-1-amine in step j. ¹H NMR (400 MHz, Chloroform-d) δ 11.99 (s, 1H), 8.64 (d, J=2.6 Hz, 1H), 8.24 (d, J=1.5 Hz, 1H), 7.83-7.78 (m, 2H), 7.67 (d, J=8.5 Hz, 1H), 7.63 (d, J=2.4 Hz, 1H), 6.99 (d, J=1.6 Hz, 1H), 4.76 (dq, J=10.4, 8.5, 8.0 Hz, 2H), 4.37 (t, J=11.8 Hz, 2H), 4.00 (t, J=11.5 Hz, 2H), 3.72 (s, 2H), 3.58-3.45 (m, 1H), 3.32 (s, 3H), 3.00 (td, J=7.4, 5.1 Hz, 1H), 2.10-1.90 (m, 3H), 1.76-1.53 (m, 3H), 1.43-1.28 (m, 1H), 1.13-1.06 (m, 2H), 1.04-0.94 (m, 2H). ESI MS [M+H]⁺ for C₃₄H₃₃F₅N₆O₄ calcd 685.3 found 685.3.

### Example 155: N-(4-{4-Chloro-2-[(3-cyano-1-azetidinyl)carbonyl]phenyl}-6-cyclopropyl-2-pyridyl)-1-cyclopropyl-5-{[(S)-2-methoxypropylamino]methyl}-2-oxo-1,2-dihydronicotinamide

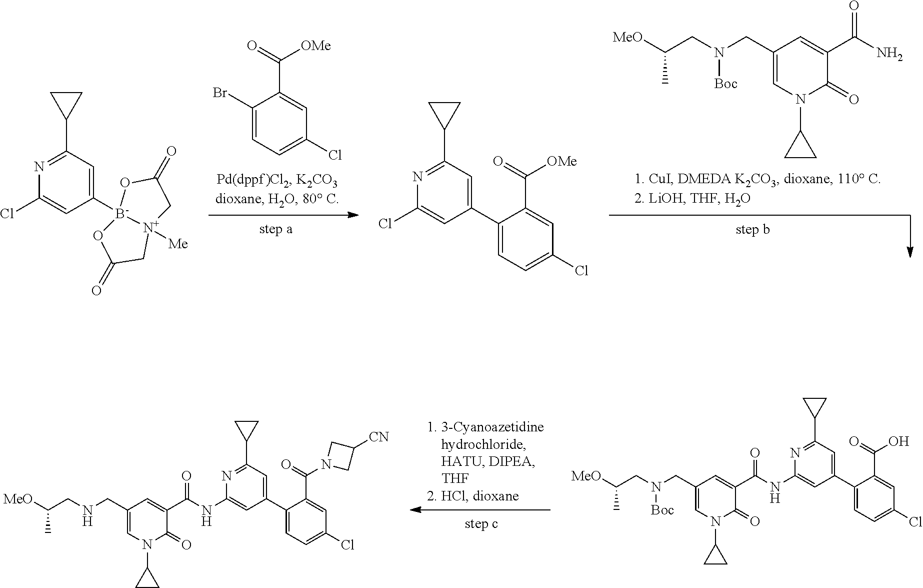 [SMILES: C[N+]12CC(=O)O[B-]1(c1cc(Cl)nc(C3CC3)c1)OC(=O)C2 ; COC(=O)c1cc(Cl)ccc1Br ; COC(=O)c1cc(Cl)ccc1-c1cc(Cl)nc(C2CC2)c1 ; CO[C@@H](C)CN(Cc1cc(C(N)=O)c(=O)n(C2CC2)c1)C(=O)OC(C)(C)C ; CO[C@@H](C)CNCc1cc(C(=O)Nc2cc(-c3ccc(Cl)cc3C(=O)N3CC(C#N)C3)cc(C3CC3)n2)c(=O)n(C2CC2)c1 ; CO[C@@H](C)CN(Cc1cc(C(=O)Nc2cc(-c3ccc(Cl)cc3C(=O)O)cc(C3CC3)n2)c(=O)n(C2CC2)c1)C(=O)OC(C)(C)C]

Step a: To a mixture of 1-(2-chloro-6-cyclopropylpyridin-4-yl)-5-methyl-2,8-dioxa-5-azonia-1-boranuidabicyclo[3.3.0]octane-3,7-dione (3.09 g, 10 mmol, 1.0 equiv., prepared according to example 15) and methyl 2-bromo-5-chlorobenzoate (2.74 g, 11 mmol, 1.1 equiv.) in dioxane (40 mL) was added Pd(dppf)Cl₂ (366 mg, 0.50 mmol, 5 mol %) and 1M K₂CO₃ aqueous solution (20 mL, 20 mmol, 2.0 equiv.). The resulting mixture was heated at 80° C. for 3 h and cooled to room temperature. The organic phase was separated, and the aqueous layer was extracted with EtOAc. The combined organic phase was dried over Na₂SO₄, concentrated, and the crude residue was purified by column chromatography (SiO₂, EtOAc in hexanes, 0 to 15%) to give the product.

Step b: To a solution of the product from step a (1.42 g, 4.4 mmol, 1.1 equiv.) and tert-butyl N-[(5-carbamoyl-1-cyclopropyl-6-oxopyridin-3-yl)methyl]-N-[(2S)-2-methoxypropyl]carbamate (the product of step b in Example 102, 1.52 g, 4.0 mmol, 1.0 equiv.) in dioxane (20 mL) was added CuI (762 mg, 4.0 mmol, 1.0 equiv.), DMEDA (0.86 mL, 705 mg, 8.0 mmol, 2.0 equiv.) and K₂CO₃ (1.66 g, 12 mmol, 3.0 equiv.). The resulting mixture was heated at 110° C. under N₂ for overnight. After cooled to room temperature, the mixture was diluted with EtOAc and water, and then separated. The aqueous phase was extracted with EtOAc twice. The combined organic solution was concentrated. The crude reaction residue was diluted with THF/H₂O (v/v 2:1, 30 mL), followed by the addition of LiOH (288 mg, 12 mmol, 3.0 equiv.). The resulting mixture was heated at 40° C. for 2 h to give a full conversion and then cooled to room temperature. The reaction mixture was acidified with 6M HCl (aq., 2 mL) and then extracted with EtOAc three times. The combined organic solution was washed with brine, dried over Na₂SO₄, and concentrated. The residue was then purified by column chromatography (C18, MeCN in H₂O, 10 to 100%, 0.1% formic acid) to give the product.

Step c: To a solution of the product from step b (40.0 mg, 0.061 mmol, 1.0 equiv.) and 3-cyanoazetidine hydrochloride (9.5 mg, 0.080 mmol, 1.3 equiv.) in THF (1.0 mL) was added HATU (30.4 mg, 0.080 mmol, 1.3 equiv.) and DIPEA (23.3 mg, 0.18 mmol, 3.0 equiv.) at room temperature. The resulting mixture was stirred at room temperature for 2 h and then 4M HCl in dioxane (1 mL) was added. The reaction mixture was then stirred at room temperature for another 2 h to give a full conversion. The mixture was quenched with saturated K₂CO₃ aqueous solution and then extracted with EtOAc (2×25 mL). The combined organic solution was washed with brine, dried over Na₂SO₄, and concentrated. The residue was then purified by HPLC to afford the title compound. ¹H NMR (400 MHz, DMSO-d₆) δ 12.51 (s, 1H), 8.56 (d, J=2.6 Hz, 1H), 8.10 (d, J=1.5 Hz, 1H), 7.92 (d, J=2.6 Hz, 1H), 7.70-7.64 (m, 2H), 7.63-7.58 (m, 1H), 7.12 (d, J=1.5 Hz, 1H), 4.30-4.17 (m, 3H), 4.11 (dd, J=9.7, 6.2 Hz, 1H), 3.92 (dt, J=9.4, 6.6 Hz, 1H), 3.57 (s, 2H), 3.50 (tt, J=7.6, 4.3 Hz, 1H), 3.39-3.34 (m, 1H), 3.22 (s, 3H), 2.57-2.50 (m, 1H), 2.50-2.40 (m, 1H), 2.14 (tt, J=7.6, 5.2 Hz, 1H), 1.14-1.08 (m, 2H), 1.06 (d, J=6.1 Hz, 3H), 1.03-0.92 (m, 6H). ESI MS [M+H]⁺ for C₃₃H₃₅ClN₆O₄, calcd 615.2, found 615.3.

### Example 156: N-(4-{2-[(1-Azetidinyl)carbonyl]-4-chlorophenyl}-6-cyclopropyl-2-pyridyl)-1-cyclopropyl-5-{[(S)-2-methoxypropylamino]methyl}-2-oxo-1,2-dihydronicotinamide

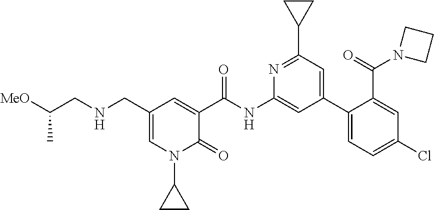 [SMILES: CO[C@@H](C)CNCc1cc(C(=O)Nc2cc(-c3ccc(Cl)cc3C(=O)N3CCC3)cc(C3CC3)n2)c(=O)n(C2CC2)c1]

The title compound was prepared in a similar fashion to that described for example 155 with azetidine hydrochloride in step c. ¹H NMR (400 MHz, DMSO-d₆) δ 12.52 (s, 1H), 8.49 (d, J=2.5 Hz, 1H), 8.13 (d, J=1.4 Hz, 1H), 7.92 (d, J=2.6 Hz, 1H), 7.63 (dd, J=8.3, 2.2 Hz, 1H), 7.57 (d, J=8.3 Hz, 1H), 7.55 (d, J=2.2 Hz, 1H), 7.08 (d, J=1.5 Hz, 1H), 3.92 (q, J=8.1 Hz, 4H), 3.57 (s, 2H), 3.50 (tt, J=7.7, 4.3 Hz, 1H), 3.42-3.30 (m, 2H), 3.22 (s, 3H), 2.57-2.50 (m, 1H), 2.45 (dd, J=12.1, 4.9 Hz, 1H), 2.32-2.19 (m, 2H), 2.13 (tt, J=7.9, 5.0 Hz, 1H), 1.14-1.08 (m, 2H), 1.06 (d, J=6.2 Hz, 3H), 1.02-0.94 (m, 6H). ESI MS [M+H]⁺ for C₃₂H₃₆ClN₅O₄, calcd 590.3, found 590.2.

### Example 157: N-[4-(4-Chloro-2-{[3-(trifluoromethyl)-1-azetidinyl]carbonyl}phenyl)-6-cyclopropyl-2-pyridyl]-1-cyclopropyl-5-{[(S)-2-methoxypropylamino]methyl}-2-oxo-1,2-dihydronicotinamide

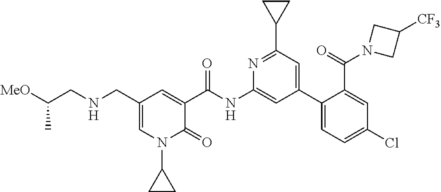 [SMILES: CO[C@@H](C)CNCc1cc(C(=O)Nc2cc(-c3ccc(Cl)cc3C(=O)N3CC(C(F)(F)F)C3)cc(C3CC3)n2)c(=O)n(C2CC2)c1]

The title compound was prepared in a similar fashion to that described for example 155 with 3-(trifluoromethyl)azetidine hydrochloride in step c. ¹H NMR (400 MHz, DMSO-d₆) δ 12.52 (s, 1H), 8.49 (d, J=2.6 Hz, 1H), 8.09 (d, J=1.4 Hz, 1H), 7.92 (d, J=2.6 Hz, 1H), 7.68 (dd, J=8.4, 2.2 Hz, 1H), 7.62-7.55 (m, 2H), 7.06 (d, J=1.5 Hz, 1H), 4.15 (q, J=10.0, 9.6 Hz, 2H), 3.82 (ddd, J=14.3, 9.9, 5.3 Hz, 2H), 3.69-3.58 (m, 1H), 3.57 (s, 2H), 3.50 (tt, J=7.7, 4.3 Hz, 1H), 3.42-3.33 (m, 1H), 3.22 (s, 3H), 2.57-2.50 (m, 1H), 2.45 (dd, J=11.9, 5.2 Hz, 1H), 2.11 (ddd, J=12.6, 8.1, 4.9 Hz, 1H), 1.16-1.06 (m, 2H), 1.06 (d, J=6.2 Hz, 3H), 1.02-0.91 (m, 6H). ESI MS [M+H]⁺ for C₃₃H₃₅ClF₃N₅O₄, calcd 658.2, found 658.3.

### Example 158: N-(4-{4-Chloro-2-[(3-fluoro-3-methyl-1-azetidinyl)carbonyl]phenyl}-6-cyclopropyl-2-pyridyl)-1-cyclopropyl-5-{[(S)-2-methoxypropylamino]methyl}-2-oxo-1,2-dihydronicotinamide

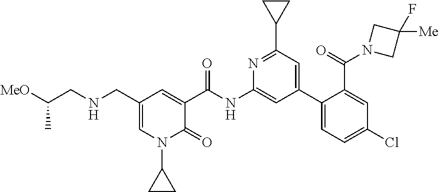 [SMILES: CO[C@@H](C)CNCc1cc(C(=O)Nc2cc(-c3ccc(Cl)cc3C(=O)N3CC(C)(F)C3)cc(C3CC3)n2)c(=O)n(C2CC2)c1]

The title compound was prepared in a similar fashion to that described for example 155 with 3-fluoro-3-methylazetidine hydrochloride in step c. ¹H NMR (400 MHz, DMSO-d₆) δ 12.50 (s, 1H), 8.46 (d, J=2.6 Hz, 1H), 8.09 (d, J=1.4 Hz, 1H), 7.93 (d, J=2.6 Hz, 1H), 7.68-7.62 (m, 2H), 7.59 (d, J=8.1 Hz, 1H), 7.06 (d, J=1.5 Hz, 1H), 4.08-3.93 (m, 3H), 3.89 (dd, J=17.9, 10.0 Hz, 1H), 3.58 (s, 2H), 3.50 (tt, J=7.7, 4.3 Hz, 1H), 3.40-3.34 (m, 1H), 3.23 (s, 3H), 2.58-2.50 (m, 1H), 2.48-2.43 (m, 1H), 2.11 (ddd, J=12.7, 8.1, 4.8 Hz, 1H), 1.50 (d, J=22.0 Hz, 3H), 1.16-1.08 (m, 2H), 1.06 (d, J=6.1 Hz, 3H), 1.05-0.90 (m, 6H). ESI MS [M+H]⁺ for C₃₃H₃₇ClFN₅O₄, calcd 622.3, found 622.3.

### Example 159: N-(4-{4-Chloro-2-[(3-methyl-1-azetidinyl)carbonyl]phenyl}-6-cyclopropyl-2-pyridyl)-1-cyclopropyl-5-{[(S)-2-methoxypropylamino]methyl}-2-oxo-1,2-dihydronicotinamide

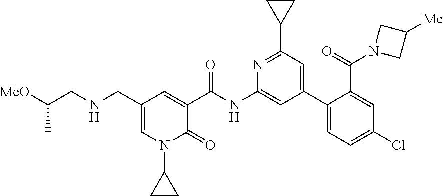 [SMILES: CO[C@@H](C)CNCc1cc(C(=O)Nc2cc(-c3ccc(Cl)cc3C(=O)N3CC(C)C3)cc(C3CC3)n2)c(=O)n(C2CC2)c1]

The title compound was prepared in a similar fashion to that described for example 155 with 3-methylazetidine hydrochloride in step c. ¹H NMR (400 MHz, DMSO-d₆) δ 12.51 (s, 1H), 8.48 (d, J=2.6 Hz, 1H), 8.13 (d, J=1.4 Hz, 1H), 7.91 (d, J=2.6 Hz, 1H), 7.63 (dd, J=8.4, 2.2 Hz, 1H), 7.59-7.54 (m, 2H), 7.07 (d, J=1.5 Hz, 1H), 3.99 (q, J=8.7, 8.2 Hz, 2H), 3.57 (s, 2H), 3.50 (tt, J=7.5, 4.3 Hz, 1H), 3.45-3.33 (m, 3H), 3.22 (s, 3H), 2.71-2.59 (m, 1H), 2.57-2.50 (m, 1H), 2.45 (dd, J=12.1, 5.0 Hz, 1H), 2.12 (ddd, J=12.5, 7.9, 4.9 Hz, 1H), 1.15-1.07 (m, 2H), 1.10-1.03 (m, 6H), 1.02-0.93 (m, 6H). ESI MS [M+H]⁺ for C₃₃H₃₈ClN₅O₄, calcd 604.3, found 604.3.

### Example 160: N-(4-{4-Chloro-2-[(2-oxa-6-aza-6-spiro[3.3]heptyl)carbonyl]phenyl}-6-cyclopropyl-2-pyridyl)-1-cyclopropyl-5-{[(S)-2-methoxypropylamino]methyl}-2-oxo-1,2-dihydronicotinamide

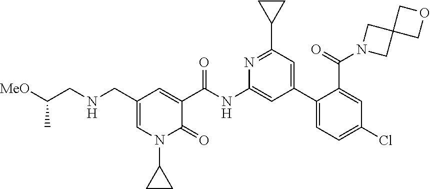 [SMILES: CO[C@@H](C)CNCc1cc(C(=O)Nc2cc(-c3ccc(Cl)cc3C(=O)N3CC4(COC4)C3)cc(C3CC3)n2)c(=O)n(C2CC2)c1]

The title compound was prepared in a similar fashion to that described for example 155 with 2-oxa-6-azaspiro[3.3]heptane in step c. ¹H NMR (400 MHz, DMSO-d₆) δ 12.50 (s, 1H), 8.45 (d, J=2.6 Hz, 1H), 8.07 (d, J=1.4 Hz, 1H), 7.92 (d, J=2.5 Hz, 1H), 7.67-7.57 (m, 3H), 7.10 (d, J=1.5 Hz, 1H), 4.75-4.60 (m, 4H), 4.20 (s, 2H), 4.10 (s, 2H), 3.57 (s, 2H), 3.50 (tt, J=7.6, 4.3 Hz, 1H), 3.39-3.35 (m, 1H), 3.22 (s, 3H), 2.56-2.50 (m, 1H), 2.44 (dd, J=12.0, 4.9 Hz, 1H), 2.12 (tt, J=7.9, 4.9 Hz, 1H), 1.16-1.06 (m, 2H), 1.05 (d, J=6.2 Hz, 3H), 1.02-0.91 (m, 6H). ESI MS [M+H]⁺ for C₃₄H₃₈ClN₅O₅, calcd 632.3, found 632.3.

### Example 161: N-{4-[4-Chloro-2-(N,N-dimethylcarbamoyl)phenyl]-6-cyclopropyl-2-pyridyl}-1-cyclopropyl-5-{[(S)-2-methoxypropylamino]methyl}-2-oxo-1,2-dihydronicotinamide

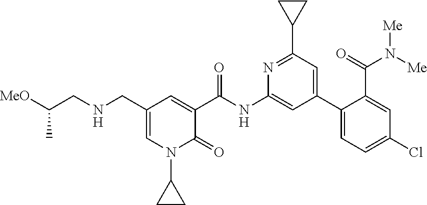 [SMILES: CO[C@@H](C)CNCc1cc(C(=O)Nc2cc(-c3ccc(Cl)cc3C(=O)N(C)C)cc(C3CC3)n2)c(=O)n(C2CC2)c1]

The title compound was prepared in a similar fashion to that described for example 155 with dimethylamine hydrochloride in step c. ¹H NMR (400 MHz, DMSO-d₆) δ 12.49 (s, 1H), 8.48 (d, J=2.6 Hz, 1H), 8.05 (d, J=1.4 Hz, 1H), 7.92 (d, J=2.5 Hz, 1H), 7.62 (dd, J=8.3, 2.1 Hz, 1H), 7.58 (d, J=8.3 Hz, 1H), 7.50 (d, J=2.1 Hz, 1H), 7.03 (d, J=1.5 Hz, 1H), 3.59 (s, 2H), 3.50 (tt, J=7.6, 4.3 Hz, 1H), 3.37 (td, J=6.3, 4.9 Hz, 1H), 3.23 (s, 3H), 2.85 (s, 3H), 2.78 (s, 3H), 2.59-2.44 (m, 2H), 2.11 (ddd, J=12.6, 8.0, 4.9 Hz, 1H), 1.15-1.08 (m, 2H), 1.06 (d, J=6.2 Hz, 3H), 1.03-0.83 (m, 6H). ESI MS [M+H]⁺ for C₃₁H₃₆ClN₅O₄, calcd 578.3, found 578.2.

Examples 162-421 were prepared in an analogous manner as procedures described herein from the appropriate starting materials and are shown in Table 6.

TABLE 6

| Example | | [M + H] | [M + H] |
|---|---|---|---|
| # | Structure | calcd. | found |
| 162 | 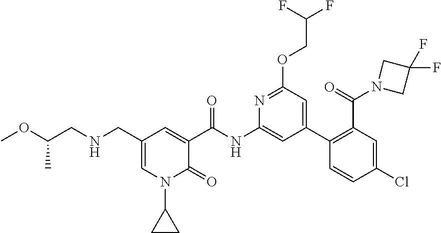 [SMILES: CO[C@@H](C)CNCc1cc(C(=O)Nc2cc(-c3ccc(Cl)cc3C(=O)N3CC(F)(F)C3)cc(OCC(F)F)n2)c(=O)n(C2CC2)c1] | 666.2 | 666.1 |
| 163 | 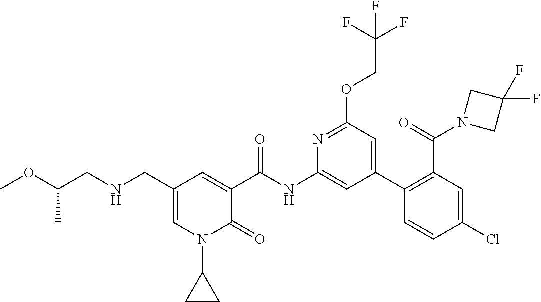 [SMILES: CO[C@@H](C)CNCc1cc(C(=O)Nc2cc(-c3ccc(Cl)cc3C(=O)N3CC(F)(F)C3)cc(OCC(F)(F)F)n2)c(=O)n(C2CC2)c1] | 684.2 | 684.1 |
| 164 | 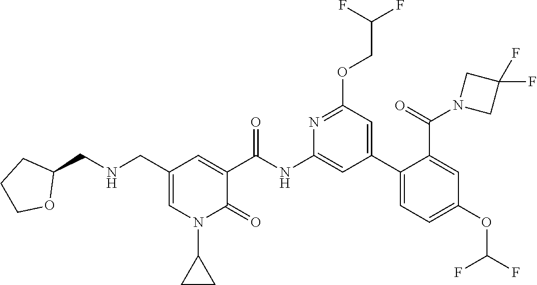 [SMILES: O=C(Nc1cc(-c2ccc(OC(F)F)cc2C(=O)N2CC(F)(F)C2)cc(OCC(F)F)n1)c1cc(CNC[C@@H]2CCCO2)cn(C2CC2)c1=O] | 710.2 | 710.2 |
| 165 | 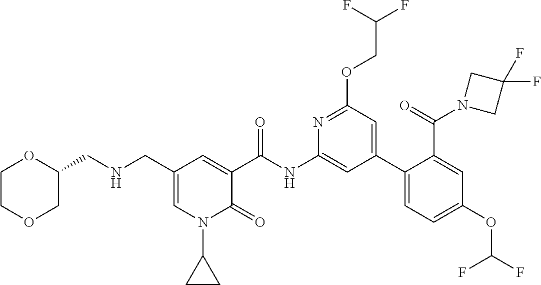 [SMILES: O=C(Nc1cc(-c2ccc(OC(F)F)cc2C(=O)N2CC(F)(F)C2)cc(OCC(F)F)n1)c1cc(CNC[C@@H]2COCCO2)cn(C2CC2)c1=O] | 726.2 | 726.2 |
| 166 | 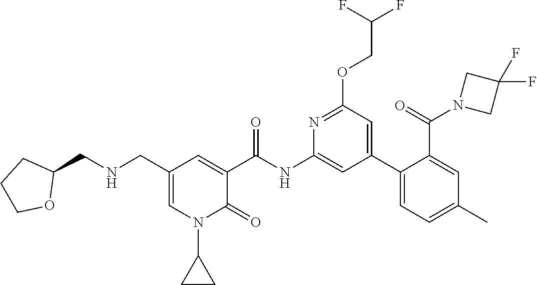 [SMILES: Cc1ccc(-c2cc(NC(=O)c3cc(CNC[C@@H]4CCCO4)cn(C4CC4)c3=O)nc(OCC(F)F)c2)c(C(=O)N2CC(F)(F)C2)c1] | 658.3 | 658.2 |
| 167 | 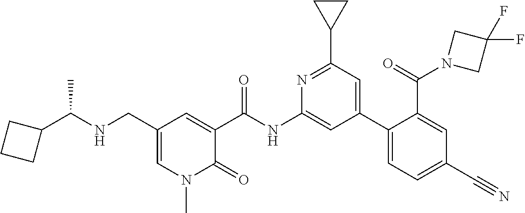 [SMILES: C[C@H](NCc1cc(C(=O)Nc2cc(-c3ccc(C#N)cc3C(=O)N3CC(F)(F)C3)cc(C3CC3)n2)c(=O)n(C)c1)C1CCC1] | 601.3 | 601.3 |
| 168 | 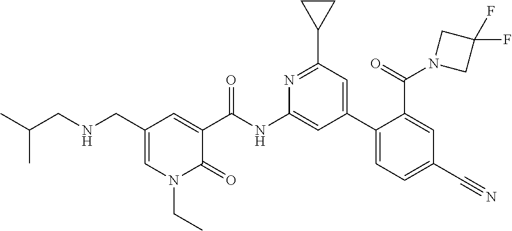 [SMILES: CCn1cc(CNCC(C)C)cc(C(=O)Nc2cc(-c3ccc(C#N)cc3C(=O)N3CC(F)(F)C3)cc(C3CC3)n2)c1=O] | 589.3 | 589.3 |
| 169 | 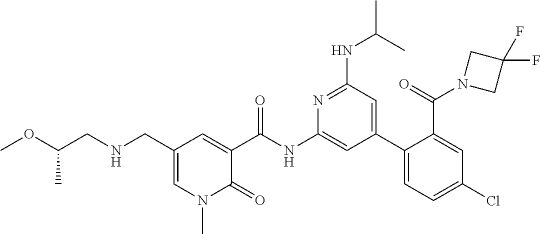 [SMILES: CO[C@@H](C)CNCc1cc(C(=O)Nc2cc(-c3ccc(Cl)cc3C(=O)N3CC(F)(F)C3)cc(NC(C)C)n2)c(=O)n(C)c1] | 617.2 | 617.2 |
| 170 | 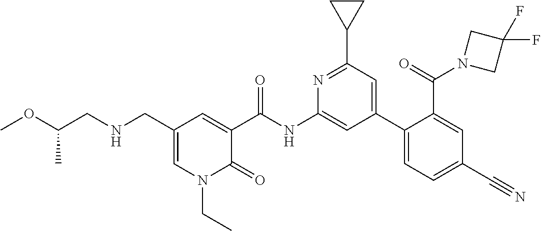 [SMILES: CCn1cc(CNC[C@H](C)OC)cc(C(=O)Nc2cc(-c3ccc(C#N)cc3C(=O)N3CC(F)(F)C3)cc(C3CC3)n2)c1=O] | 605.3 | 605.3 |
| 171 | 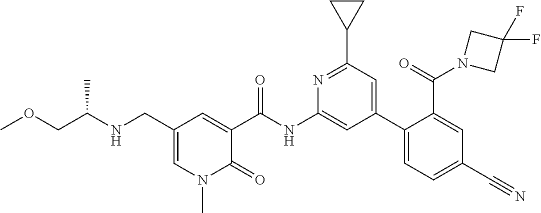 [SMILES: COC[C@H](C)NCc1cc(C(=O)Nc2cc(-c3ccc(C#N)cc3C(=O)N3CC(F)(F)C3)cc(C3CC3)n2)c(=O)n(C)c1] | 591.2 | 591.3 |
| 172 | 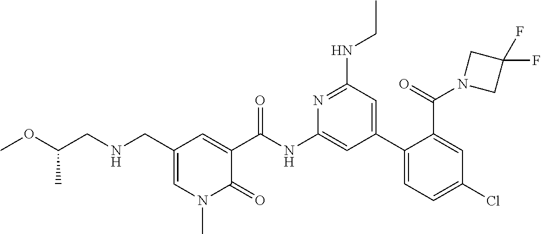 [SMILES: CCNc1cc(-c2ccc(Cl)cc2C(=O)N2CC(F)(F)C2)cc(NC(=O)c2cc(CNC[C@H](C)OC)cn(C)c2=O)n1] | 603.2 | 603.2 |
| 173 | 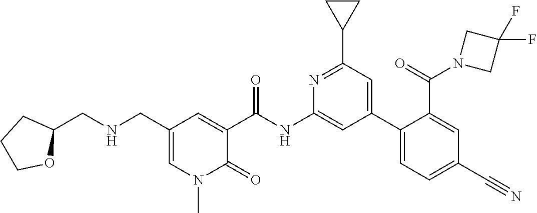 [SMILES: Cn1cc(CNC[C@@H]2CCCO2)cc(C(=O)Nc2cc(-c3ccc(C#N)cc3C(=O)N3CC(F)(F)C3)cc(C3CC3)n2)c1=O] | 603.2 | 603.3 |
| 174 | 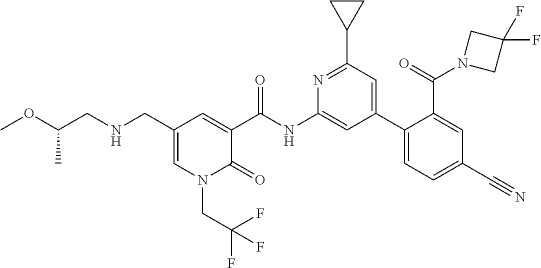 [SMILES: CO[C@@H](C)CNCc1cc(C(=O)Nc2cc(-c3ccc(C#N)cc3C(=O)N3CC(F)(F)C3)cc(C3CC3)n2)c(=O)n(CC(F)(F)F)c1] | 659.2 | 659.3 |
| 175 | 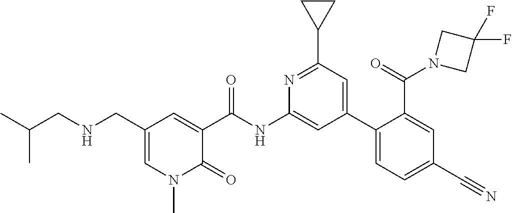 [SMILES: CC(C)CNCc1cc(C(=O)Nc2cc(-c3ccc(C#N)cc3C(=O)N3CC(F)(F)C3)cc(C3CC3)n2)c(=O)n(C)c1] | 575.3 | 575.3 |
| 176 | 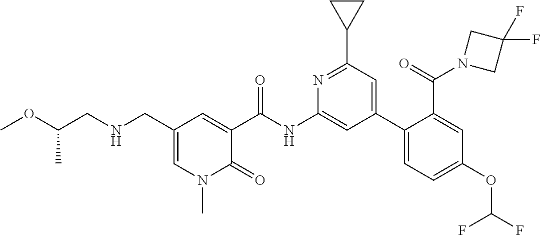 [SMILES: CO[C@@H](C)CNCc1cc(C(=O)Nc2cc(-c3ccc(OC(F)F)cc3C(=O)N3CC(F)(F)C3)cc(C3CC3)n2)c(=O)n(C)c1] | 632.2 | 632.3 |
| 177 | 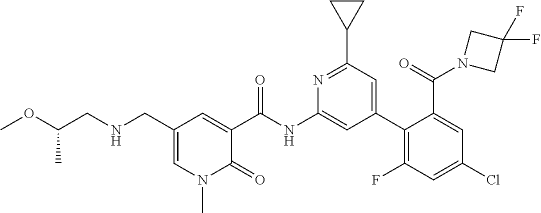 [SMILES: CO[C@@H](C)CNCc1cc(C(=O)Nc2cc(-c3c(F)cc(Cl)cc3C(=O)N3CC(F)(F)C3)cc(C3CC3)n2)c(=O)n(C)c1] | 618.2 | 618.2 |
| 178 | 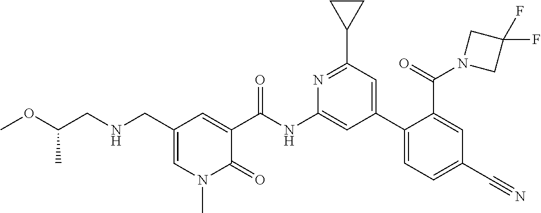 [SMILES: CO[C@@H](C)CNCc1cc(C(=O)Nc2cc(-c3ccc(C#N)cc3C(=O)N3CC(F)(F)C3)cc(C3CC3)n2)c(=O)n(C)c1] | 591.2 | 591.3 |
| 179 | 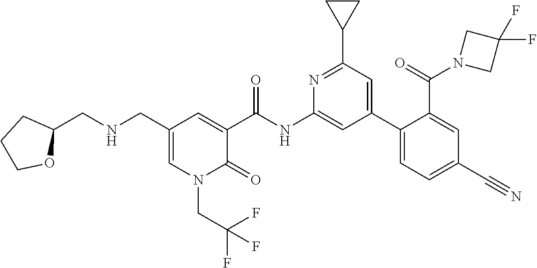 [SMILES: N#Cc1ccc(-c2cc(NC(=O)c3cc(CNC[C@@H]4CCCO4)cn(CC(F)(F)F)c3=O)nc(C3CC3)c2)c(C(=O)N2CC(F)(F)C2)c1] | 671.2 | 671.3 |
| 180 | 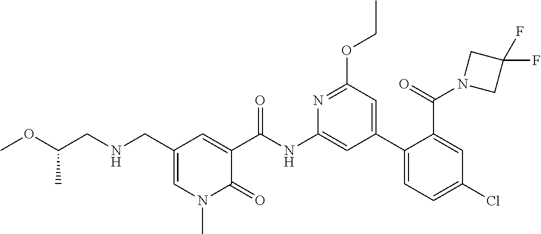 [SMILES: CCOc1cc(-c2ccc(Cl)cc2C(=O)N2CC(F)(F)C2)cc(NC(=O)c2cc(CNC[C@H](C)OC)cn(C)c2=O)n1] | 604.2 | 604.2 |
| 181 | 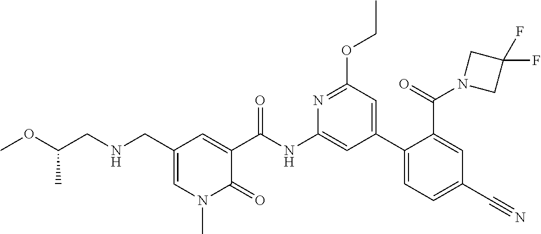 [SMILES: CCOc1cc(-c2ccc(C#N)cc2C(=O)N2CC(F)(F)C2)cc(NC(=O)c2cc(CNC[C@H](C)OC)cn(C)c2=O)n1] | 595.2 | 595.3 |
| 182 | 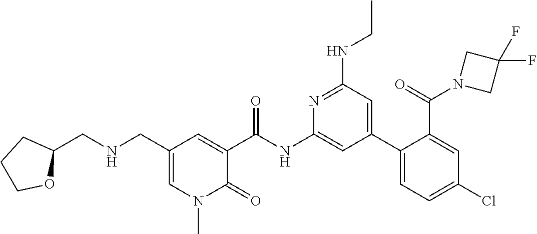 [SMILES: CCNc1cc(-c2ccc(Cl)cc2C(=O)N2CC(F)(F)C2)cc(NC(=O)c2cc(CNC[C@@H]3CCCO3)cn(C)c2=O)n1] | 615.2 | 615.2 |
| 183 | 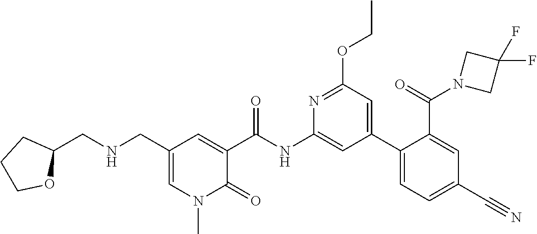 [SMILES: CCOc1cc(-c2ccc(C#N)cc2C(=O)N2CC(F)(F)C2)cc(NC(=O)c2cc(CNC[C@@H]3CCCO3)cn(C)c2=O)n1] | 607.2 | 607.3 |
| 184 | 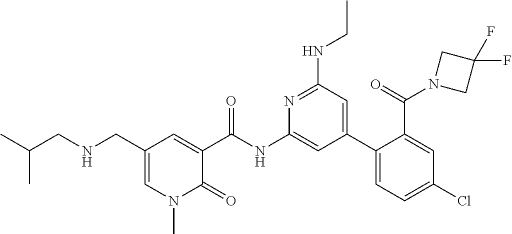 [SMILES: CCNc1cc(-c2ccc(Cl)cc2C(=O)N2CC(F)(F)C2)cc(NC(=O)c2cc(CNCC(C)C)cn(C)c2=O)n1] | 587.2 | 587.2 |
| 185 | 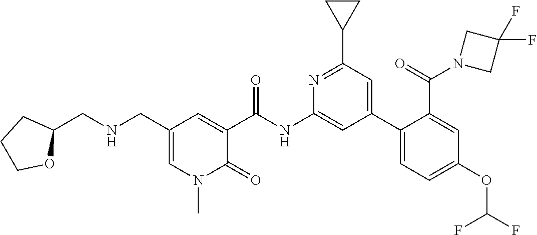 [SMILES: Cn1cc(CNC[C@@H]2CCCO2)cc(C(=O)Nc2cc(-c3ccc(OC(F)F)cc3C(=O)N3CC(F)(F)C3)cc(C3CC3)n2)c1=O] | 644.2 | 644.3 |
| 186 | 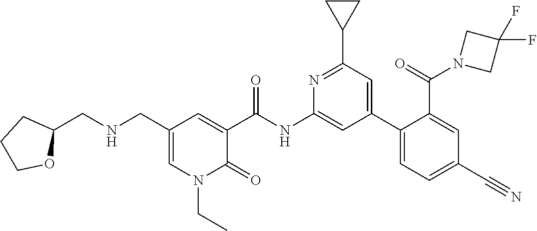 [SMILES: CCn1cc(CNC[C@@H]2CCCO2)cc(C(=O)Nc2cc(-c3ccc(C#N)cc3C(=O)N3CC(F)(F)C3)cc(C3CC3)n2)c1=O] | 617.3 | 617.3 |
| 187 | 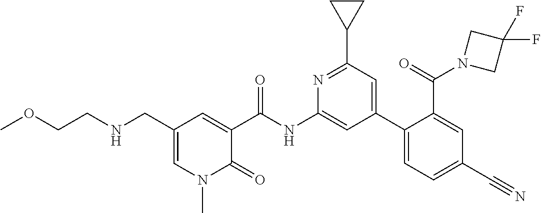 [SMILES: COCCNCc1cc(C(=O)Nc2cc(-c3ccc(C#N)cc3C(=O)N3CC(F)(F)C3)cc(C3CC3)n2)c(=O)n(C)c1] | 577.2 | 577.3 |
| 188 | 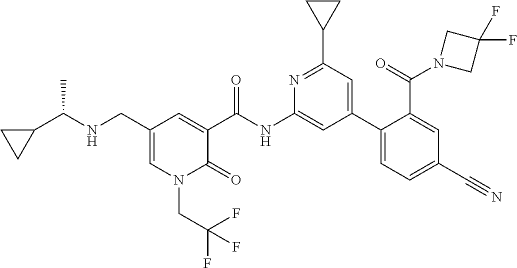 [SMILES: C[C@H](NCc1cc(C(=O)Nc2cc(-c3ccc(C#N)cc3C(=O)N3CC(F)(F)C3)cc(C3CC3)n2)c(=O)n(CC(F)(F)F)c1)C1CC1] | 655.2 | 655.2 |
| 189 | 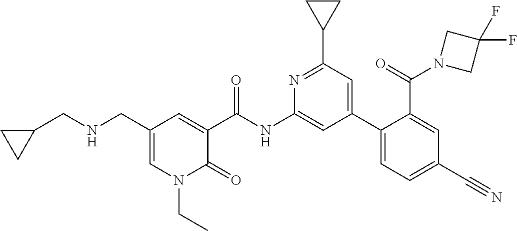 [SMILES: CCn1cc(CNCC2CC2)cc(C(=O)Nc2cc(-c3ccc(C#N)cc3C(=O)N3CC(F)(F)C3)cc(C3CC3)n2)c1=O] | 587.3 | 587.3 |
| 190 | 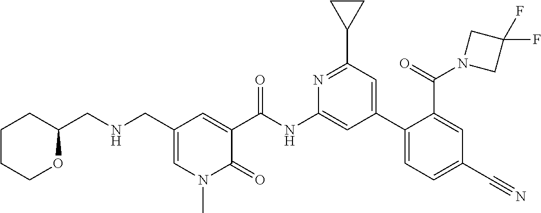 [SMILES: Cn1cc(CNC[C@@H]2CCCCO2)cc(C(=O)Nc2cc(-c3ccc(C#N)cc3C(=O)N3CC(F)(F)C3)cc(C3CC3)n2)c1=O] | 617.3 | 617.3 |
| 191 | 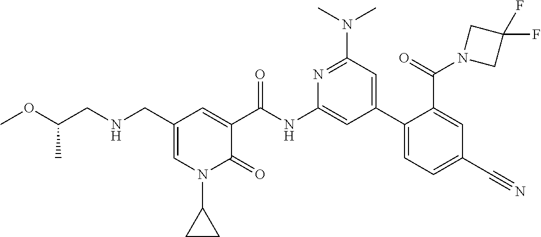 [SMILES: CO[C@@H](C)CNCc1cc(C(=O)Nc2cc(-c3ccc(C#N)cc3C(=O)N3CC(F)(F)C3)cc(N(C)C)n2)c(=O)n(C2CC2)c1] | 620.3 | 620.3 |
| 192 | 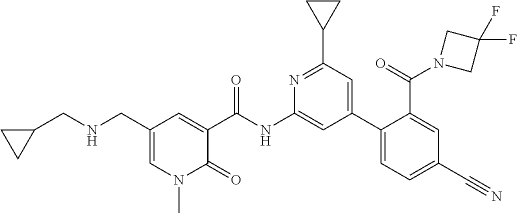 [SMILES: Cn1cc(CNCC2CC2)cc(C(=O)Nc2cc(-c3ccc(C#N)cc3C(=O)N3CC(F)(F)C3)cc(C3CC3)n2)c1=O] | 573.2 | 573.3 |
| 193 | 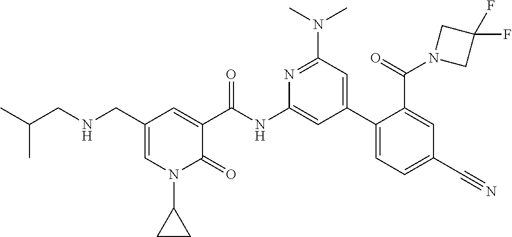 [SMILES: CC(C)CNCc1cc(C(=O)Nc2cc(-c3ccc(C#N)cc3C(=O)N3CC(F)(F)C3)cc(N(C)C)n2)c(=O)n(C2CC2)c1] | 604.3 | 604.3 |
| 194 | 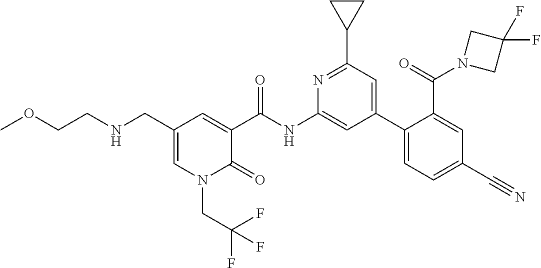 [SMILES: COCCNCc1cc(C(=O)Nc2cc(-c3ccc(C#N)cc3C(=O)N3CC(F)(F)C3)cc(C3CC3)n2)c(=O)n(CC(F)(F)F)c1] | 645.2 | 645.2 |
| 195 | 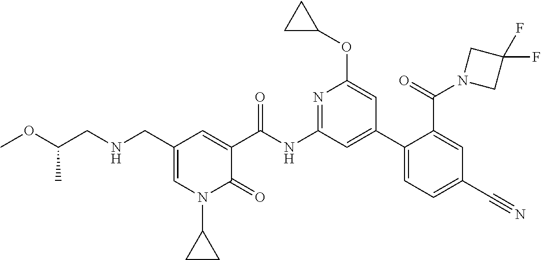 [SMILES: CO[C@@H](C)CNCc1cc(C(=O)Nc2cc(-c3ccc(C#N)cc3C(=O)N3CC(F)(F)C3)cc(OC3CC3)n2)c(=O)n(C2CC2)c1] | 633.3 | 633.3 |
| 196 | 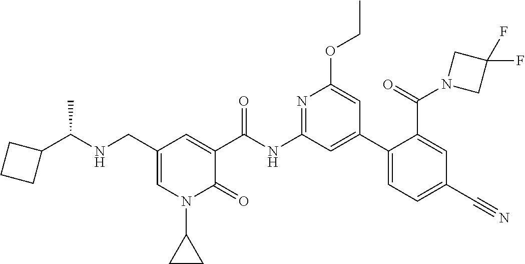 [SMILES: CCOc1cc(-c2ccc(C#N)cc2C(=O)N2CC(F)(F)C2)cc(NC(=O)c2cc(CN[C@@H](C)C3CCC3)cn(C3CC3)c2=O)n1] | 631.3 | 631.3 |
| 197 | 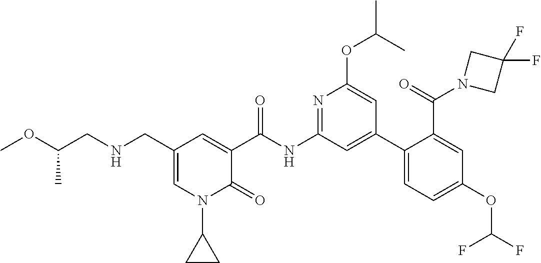 [SMILES: CO[C@@H](C)CNCc1cc(C(=O)Nc2cc(-c3ccc(OC(F)F)cc3C(=O)N3CC(F)(F)C3)cc(OC(C)C)n2)c(=O)n(C2CC2)c1] | 676.3 | 676.3 |
| 198 | 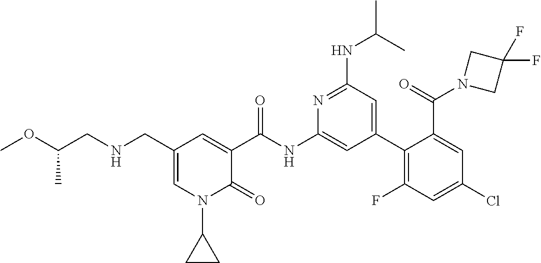 [SMILES: CO[C@@H](C)CNCc1cc(C(=O)Nc2cc(-c3c(F)cc(Cl)cc3C(=O)N3CC(F)(F)C3)cc(NC(C)C)n2)c(=O)n(C2CC2)c1] | 661.2 | 661.3 |
| 199 | 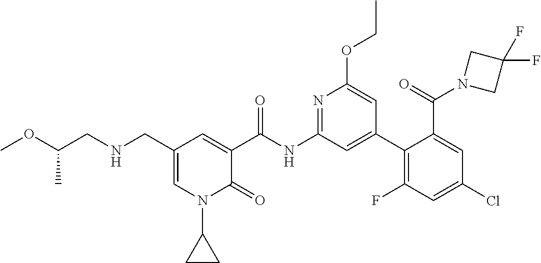 [SMILES: CCOc1cc(-c2c(F)cc(Cl)cc2C(=O)N2CC(F)(F)C2)cc(NC(=O)c2cc(CNC[C@H](C)OC)cn(C3CC3)c2=O)n1] | 648.2 | 648.3 |
| 200 | 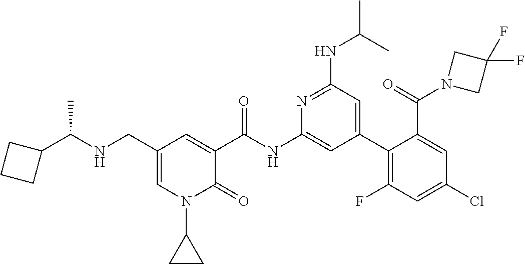 [SMILES: CC(C)Nc1cc(-c2c(F)cc(Cl)cc2C(=O)N2CC(F)(F)C2)cc(NC(=O)c2cc(CN[C@@H](C)C3CCC3)cn(C3CC3)c2=O)n1] | 671.3 | 671.3 |
| 201 | 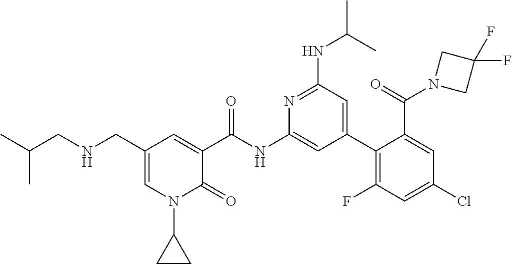 [SMILES: CC(C)CNCc1cc(C(=O)Nc2cc(-c3c(F)cc(Cl)cc3C(=O)N3CC(F)(F)C3)cc(NC(C)C)n2)c(=O)n(C2CC2)c1] | 645.2 | 645.3 |
| 202 | 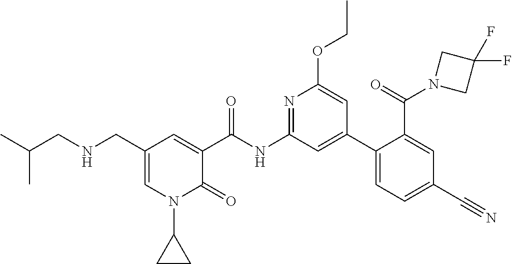 [SMILES: CCOc1cc(-c2ccc(C#N)cc2C(=O)N2CC(F)(F)C2)cc(NC(=O)c2cc(CNCC(C)C)cn(C3CC3)c2=O)n1] | 605.3 | 605.3 |
| 203 | 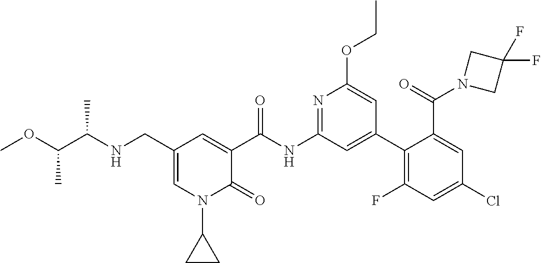 [SMILES: CCOc1cc(-c2c(F)cc(Cl)cc2C(=O)N2CC(F)(F)C2)cc(NC(=O)c2cc(CN[C@@H](C)[C@H](C)OC)cn(C3CC3)c2=O)n1] | 662.2 | 662.3 |
| 204 | 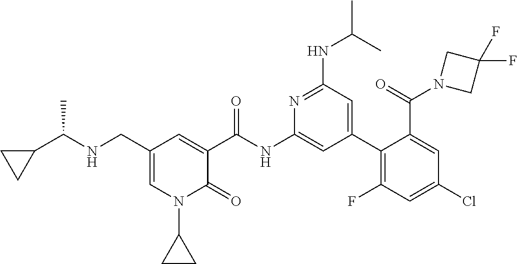 [SMILES: CC(C)Nc1cc(-c2c(F)cc(Cl)cc2C(=O)N2CC(F)(F)C2)cc(NC(=O)c2cc(CN[C@@H](C)C3CC3)cn(C3CC3)c2=O)n1] | 657.2 | 657.3 |
| 205 | 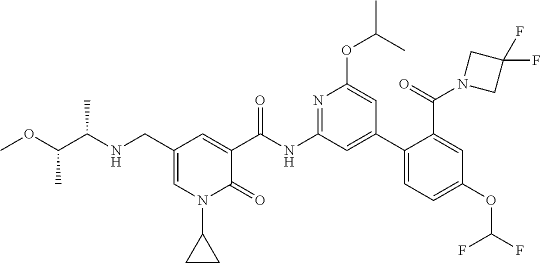 [SMILES: CO[C@@H](C)[C@H](C)NCc1cc(C(=O)Nc2cc(-c3ccc(OC(F)F)cc3C(=O)N3CC(F)(F)C3)cc(OC(C)C)n2)c(=O)n(C2CC2)c1] | 690.3 | 690.3 |
| 206 | 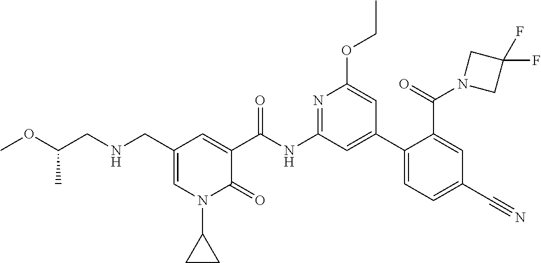 [SMILES: CCOc1cc(-c2ccc(C#N)cc2C(=O)N2CC(F)(F)C2)cc(NC(=O)c2cc(CNC[C@H](C)OC)cn(C3CC3)c2=O)n1] | 621.3 | 621.3 |
| 207 | 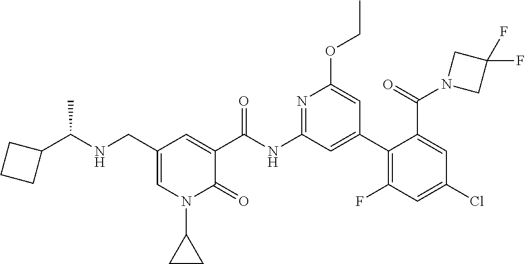 [SMILES: CCOc1cc(-c2c(F)cc(Cl)cc2C(=O)N2CC(F)(F)C2)cc(NC(=O)c2cc(CN[C@@H](C)C3CCC3)cn(C3CC3)c2=O)n1] | 658.2 | 658.3 |
| 208 | 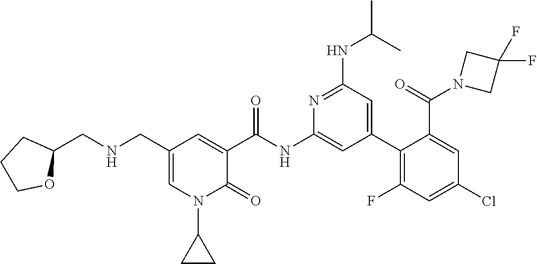 [SMILES: CC(C)Nc1cc(-c2c(F)cc(Cl)cc2C(=O)N2CC(F)(F)C2)cc(NC(=O)c2cc(CNC[C@@H]3CCCO3)cn(C3CC3)c2=O)n1] | 673.2 | 673.3 |
| 209 | 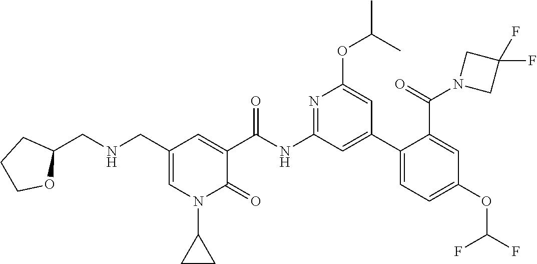 [SMILES: CC(C)Oc1cc(-c2ccc(OC(F)F)cc2C(=O)N2CC(F)(F)C2)cc(NC(=O)c2cc(CNC[C@@H]3CCCO3)cn(C3CC3)c2=O)n1] | 688.3 | 688.3 |
| 210 | 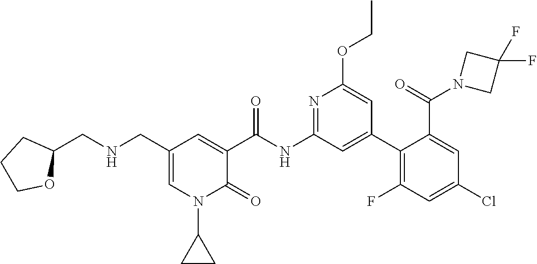 [SMILES: CCOc1cc(-c2c(F)cc(Cl)cc2C(=O)N2CC(F)(F)C2)cc(NC(=O)c2cc(CNC[C@@H]3CCCO3)cn(C3CC3)c2=O)n1] | 660.2 | 660.2 |
| 211 | 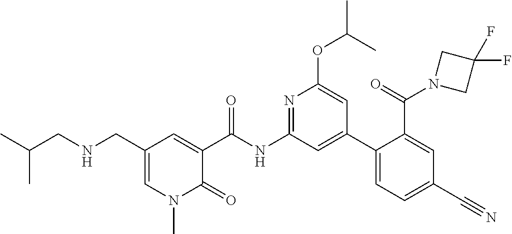 [SMILES: CC(C)CNCc1cc(C(=O)Nc2cc(-c3ccc(C#N)cc3C(=O)N3CC(F)(F)C3)cc(OC(C)C)n2)c(=O)n(C)c1] | 593.3 | 593.3 |
| 212 | 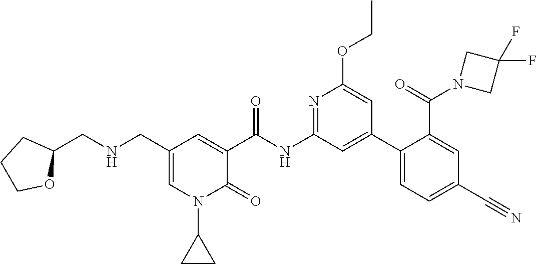 [SMILES: CCOc1cc(-c2ccc(C#N)cc2C(=O)N2CC(F)(F)C2)cc(NC(=O)c2cc(CNC[C@@H]3CCCO3)cn(C3CC3)c2=O)n1] | 633.3 | 633.3 |
| 213 | 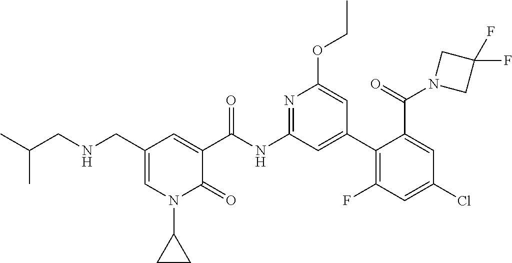 [SMILES: CCOc1cc(-c2c(F)cc(Cl)cc2C(=O)N2CC(F)(F)C2)cc(NC(=O)c2cc(CNCC(C)C)cn(C3CC3)c2=O)n1] | 632.2 | 632.3 |
| 214 | 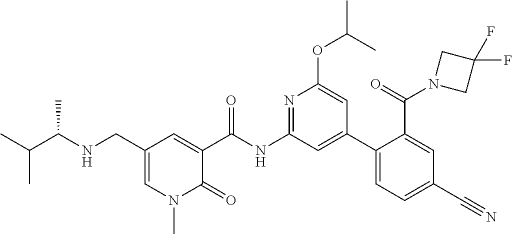 [SMILES: CC(C)Oc1cc(-c2ccc(C#N)cc2C(=O)N2CC(F)(F)C2)cc(NC(=O)c2cc(CN[C@@H](C)C(C)C)cn(C)c2=O)n1] | 607.3 | 607.3 |
| 215 | 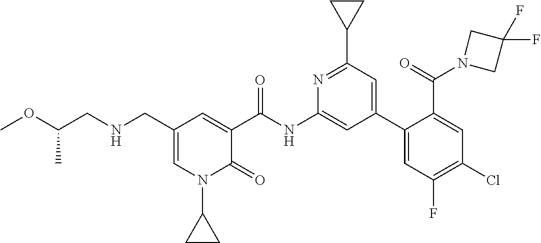 [SMILES: CO[C@@H](C)CNCc1cc(C(=O)Nc2cc(-c3cc(F)c(Cl)cc3C(=O)N3CC(F)(F)C3)cc(C3CC3)n2)c(=O)n(C2CC2)c1] | 644.2 | 644.2 |
| 216 | 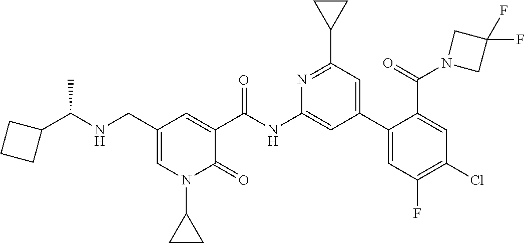 [SMILES: C[C@H](NCc1cc(C(=O)Nc2cc(-c3cc(F)c(Cl)cc3C(=O)N3CC(F)(F)C3)cc(C3CC3)n2)c(=O)n(C2CC2)c1)C1CCC1] | 654.2 | 654.3 |
| 217 | 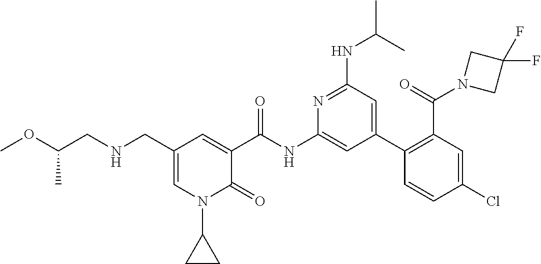 [SMILES: CO[C@@H](C)CNCc1cc(C(=O)Nc2cc(-c3ccc(Cl)cc3C(=O)N3CC(F)(F)C3)cc(NC(C)C)n2)c(=O)n(C2CC2)c1] | 643.3 | 643.2 |
| 218 | 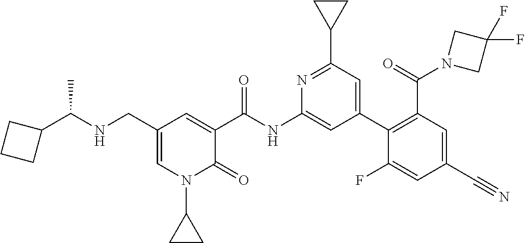 [SMILES: C[C@H](NCc1cc(C(=O)Nc2cc(-c3c(F)cc(C#N)cc3C(=O)N3CC(F)(F)C3)cc(C3CC3)n2)c(=O)n(C2CC2)c1)C1CCC1] | 645.3 | 645.2 |
| 219 | 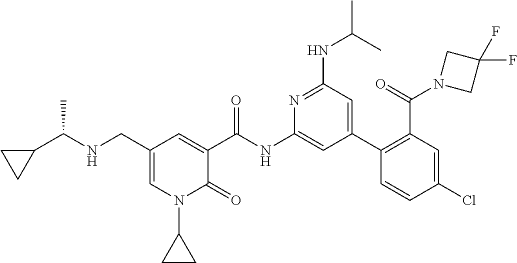 [SMILES: CC(C)Nc1cc(-c2ccc(Cl)cc2C(=O)N2CC(F)(F)C2)cc(NC(=O)c2cc(CN[C@@H](C)C3CC3)cn(C3CC3)c2=O)n1] | 639.3 | 639.3 |
| 220 | 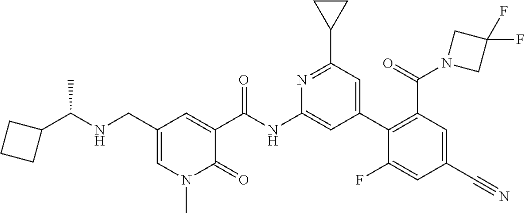 [SMILES: C[C@H](NCc1cc(C(=O)Nc2cc(-c3c(F)cc(C#N)cc3C(=O)N3CC(F)(F)C3)cc(C3CC3)n2)c(=O)n(C)c1)C1CCC1] | 619.3 | 619.2 |
| 221 | 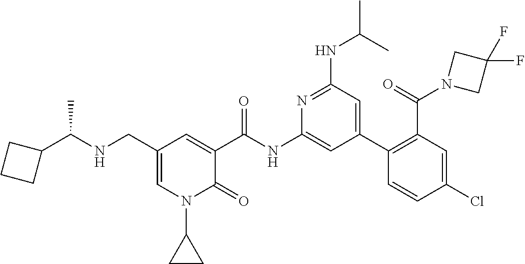 [SMILES: CC(C)Nc1cc(-c2ccc(Cl)cc2C(=O)N2CC(F)(F)C2)cc(NC(=O)c2cc(CN[C@@H](C)C3CCC3)cn(C3CC3)c2=O)n1] | 653.3 | 653.3 |
| 222 | 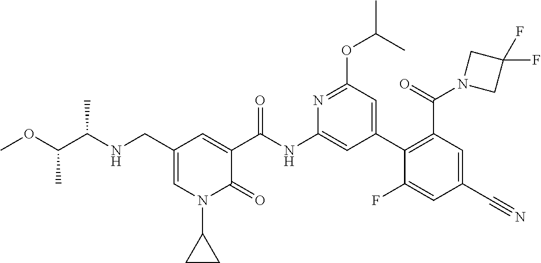 [SMILES: CO[C@@H](C)[C@H](C)NCc1cc(C(=O)Nc2cc(-c3c(F)cc(C#N)cc3C(=O)N3CC(F)(F)C3)cc(OC(C)C)n2)c(=O)n(C2CC2)c1] | 667.3 | 667.2 |
| 223 | 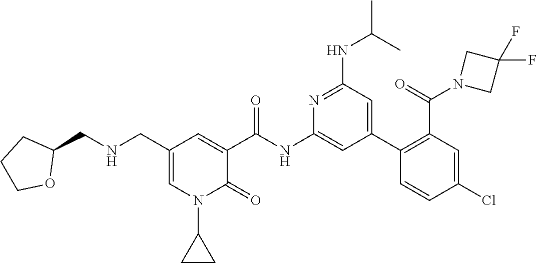 [SMILES: CC(C)Nc1cc(-c2ccc(Cl)cc2C(=O)N2CC(F)(F)C2)cc(NC(=O)c2cc(CNC[C@@H]3CCCO3)cn(C3CC3)c2=O)n1] | 655.3 | 655.3 |
| 224 | 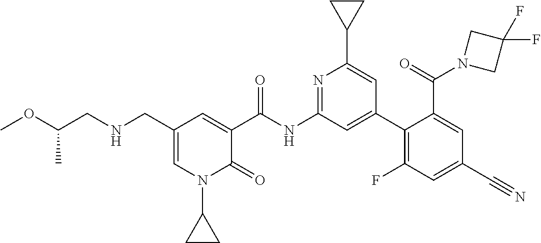 [SMILES: CO[C@@H](C)CNCc1cc(C(=O)Nc2cc(-c3c(F)cc(C#N)cc3C(=O)N3CC(F)(F)C3)cc(C3CC3)n2)c(=O)n(C2CC2)c1] | 635.3 | 635.2 |
| 225 | 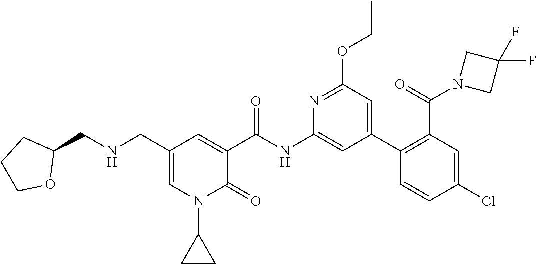 [SMILES: CCOc1cc(-c2ccc(Cl)cc2C(=O)N2CC(F)(F)C2)cc(NC(=O)c2cc(CNC[C@@H]3CCCO3)cn(C3CC3)c2=O)n1] | 642.2 | 642.2 |
| 226 | 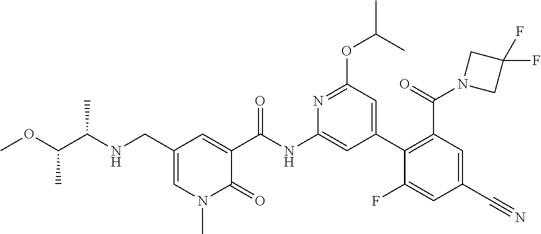 [SMILES: CO[C@@H](C)[C@H](C)NCc1cc(C(=O)Nc2cc(-c3c(F)cc(C#N)cc3C(=O)N3CC(F)(F)C3)cc(OC(C)C)n2)c(=O)n(C)c1] | 641.3 | 641.3 |
| 227 | 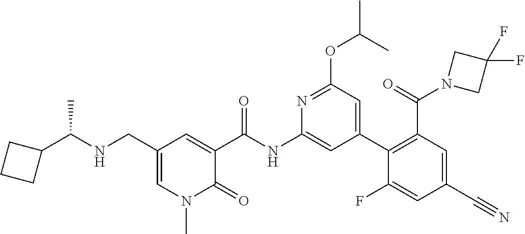 [SMILES: CC(C)Oc1cc(-c2c(F)cc(C#N)cc2C(=O)N2CC(F)(F)C2)cc(NC(=O)c2cc(CN[C@@H](C)C3CCC3)cn(C)c2=O)n1] | 637.3 | 637.3 |
| 228 | 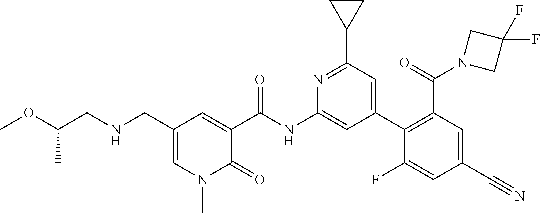 [SMILES: CO[C@@H](C)CNCc1cc(C(=O)Nc2cc(-c3c(F)cc(C#N)cc3C(=O)N3CC(F)(F)C3)cc(C3CC3)n2)c(=O)n(C)c1] | 609.2 | 609.2 |
| 229 | 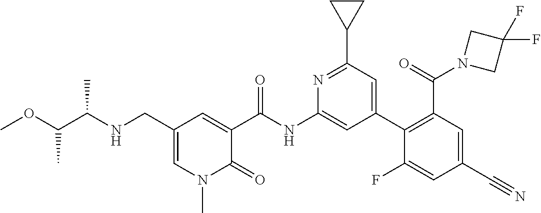 | 623.3 | 623.3 |
| 230 | 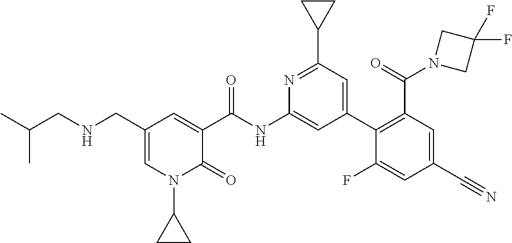 [SMILES: CC(C)CNCc1cc(C(=O)Nc2cc(-c3c(F)cc(C#N)cc3C(=O)N3CC(F)(F)C3)cc(C3CC3)n2)c(=O)n(C2CC2)c1] | 619.3 | 619.3 |
| 231 | 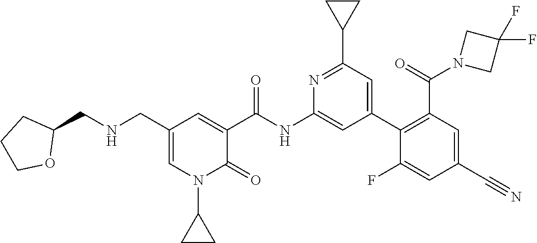 [SMILES: N#Cc1cc(F)c(-c2cc(NC(=O)c3cc(CNC[C@@H]4CCCO4)cn(C4CC4)c3=O)nc(C3CC3)c2)c(C(=O)N2CC(F)(F)C2)c1] | 647.3 | 647.3 |
| 232 | 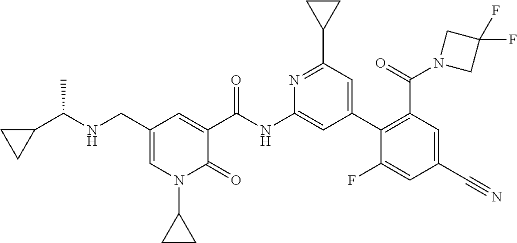 [SMILES: C[C@H](NCc1cc(C(=O)Nc2cc(-c3c(F)cc(C#N)cc3C(=O)N3CC(F)(F)C3)cc(C3CC3)n2)c(=O)n(C2CC2)c1)C1CC1] | 631.3 | 631.3 |
| 233 | 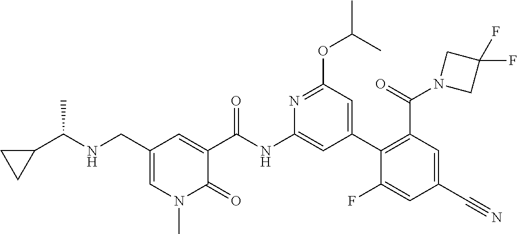 [SMILES: CC(C)Oc1cc(-c2c(F)cc(C#N)cc2C(=O)N2CC(F)(F)C2)cc(NC(=O)c2cc(CN[C@@H](C)C3CC3)cn(C)c2=O)n1] | 623.3 | 623.3 |
| 234 | 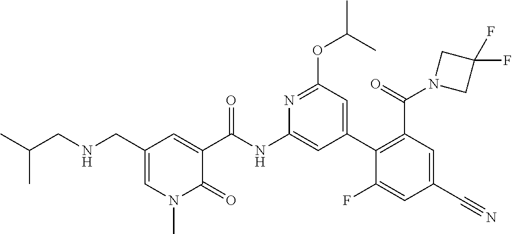 [SMILES: CC(C)CNCc1cc(C(=O)Nc2cc(-c3c(F)cc(C#N)cc3C(=O)N3CC(F)(F)C3)cc(OC(C)C)n2)c(=O)n(C)c1] | 611.3 | 611.3 |
| 235 | 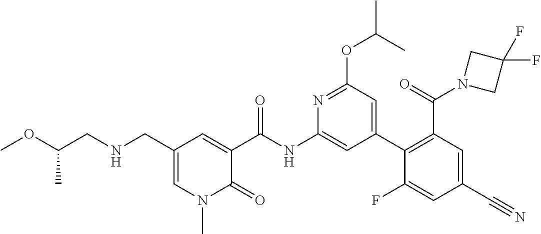 [SMILES: CO[C@@H](C)CNCc1cc(C(=O)Nc2cc(-c3c(F)cc(C#N)cc3C(=O)N3CC(F)(F)C3)cc(OC(C)C)n2)c(=O)n(C)c1] | 627.2 | 627.3 |
| 236 | 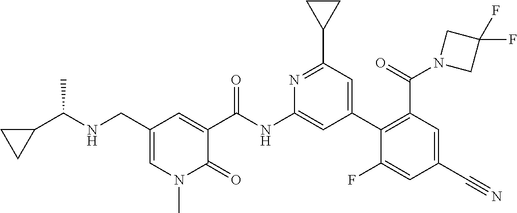 [SMILES: C[C@H](NCc1cc(C(=O)Nc2cc(-c3c(F)cc(C#N)cc3C(=O)N3CC(F)(F)C3)cc(C3CC3)n2)c(=O)n(C)c1)C1CC1] | 605.2 | 605.3 |
| 237 | 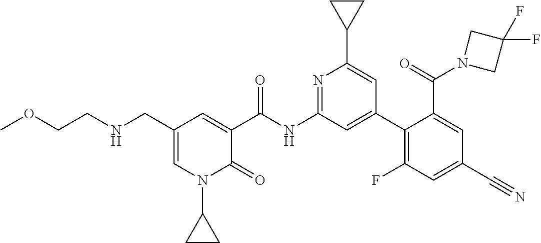 [SMILES: COCCNCc1cc(C(=O)Nc2cc(-c3c(F)cc(C#N)cc3C(=O)N3CC(F)(F)C3)cc(C3CC3)n2)c(=O)n(C2CC2)c1] | 621.2 | 621.2 |
| 238 | 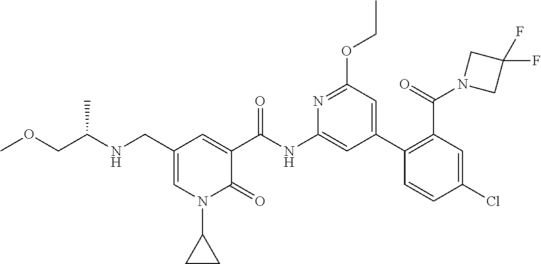 [SMILES: CCOc1cc(-c2ccc(Cl)cc2C(=O)N2CC(F)(F)C2)cc(NC(=O)c2cc(CN[C@@H](C)COC)cn(C3CC3)c2=O)n1] | 630.2 | 630.3 |
| 239 | 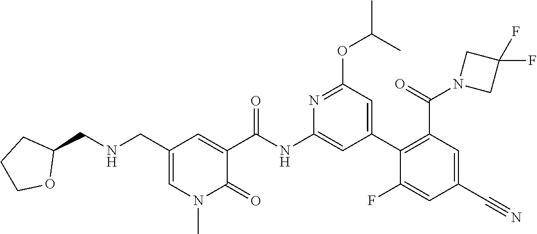 [SMILES: CC(C)Oc1cc(-c2c(F)cc(C#N)cc2C(=O)N2CC(F)(F)C2)cc(NC(=O)c2cc(CNC[C@@H]3CCCO3)cn(C)c2=O)n1] | 639.2 | 639.3 |
| 240 | 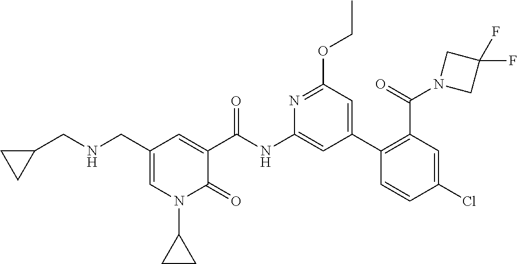 [SMILES: CCOc1cc(-c2ccc(Cl)cc2C(=O)N2CC(F)(F)C2)cc(NC(=O)c2cc(CNCC3CC3)cn(C3CC3)c2=O)n1] | 612.2 | 612.2 |
| 241 | 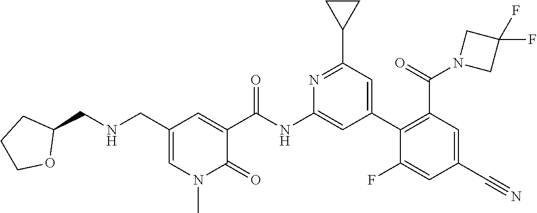 [SMILES: Cn1cc(CNC[C@@H]2CCCO2)cc(C(=O)Nc2cc(-c3c(F)cc(C#N)cc3C(=O)N3CC(F)(F)C3)cc(C3CC3)n2)c1=O] | 621.2 | 621.2 |
| 242 | 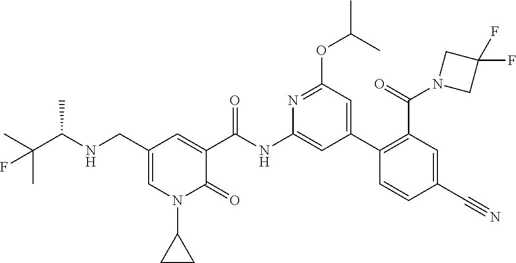 [SMILES: CC(C)Oc1cc(-c2ccc(C#N)cc2C(=O)N2CC(F)(F)C2)cc(NC(=O)c2cc(CN[C@@H](C)C(C)(C)F)cn(C3CC3)c2=O)n1] | 651.3 | 651.3 |
| 243 | 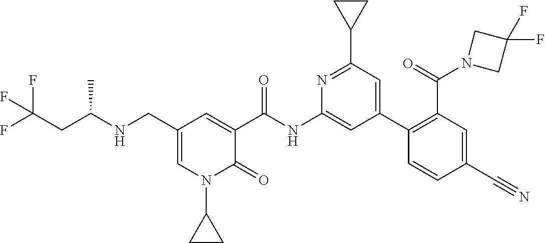 [SMILES: C[C@@H](CC(F)(F)F)NCc1cc(C(=O)Nc2cc(-c3ccc(C#N)cc3C(=O)N3CC(F)(F)C3)cc(C3CC3)n2)c(=O)n(C2CC2)c1] | 655.2 | 655.3 |
| 244 | 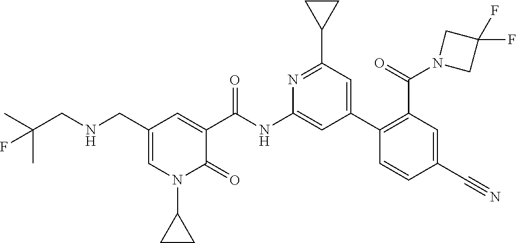 [SMILES: CC(C)(F)CNCc1cc(C(=O)Nc2cc(-c3ccc(C#N)cc3C(=O)N3CC(F)(F)C3)cc(C3CC3)n2)c(=O)n(C2CC2)c1] | 619.3 | 619.3 |
| 245 | 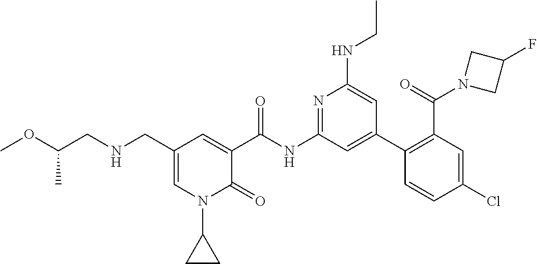 [SMILES: CCNc1cc(-c2ccc(Cl)cc2C(=O)N2CC(F)C2)cc(NC(=O)c2cc(CNC[C@H](C)OC)cn(C3CC3)c2=O)n1] | 611.2 | 611.3 |
| 246 | 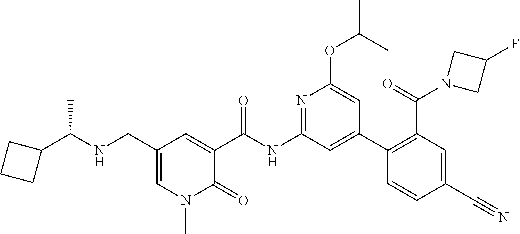 [SMILES: CC(C)Oc1cc(-c2ccc(C#N)cc2C(=O)N2CC(F)C2)cc(NC(=O)c2cc(CN[C@@H](C)C3CCC3)cn(C)c2=O)n1] | 601.3 | 601.3 |
| 247 | 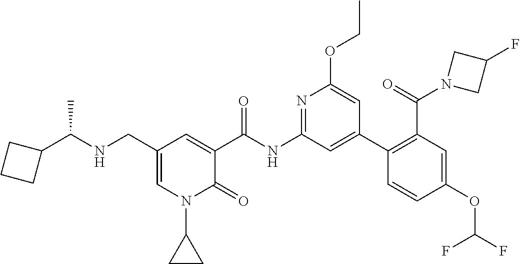 [SMILES: CCOc1cc(-c2ccc(OC(F)F)cc2C(=O)N2CC(F)C2)cc(NC(=O)c2cc(CN[C@@H](C)C3CCC3)cn(C3CC3)c2=O)n1] | 654.3 | 654.3 |
| 248 | 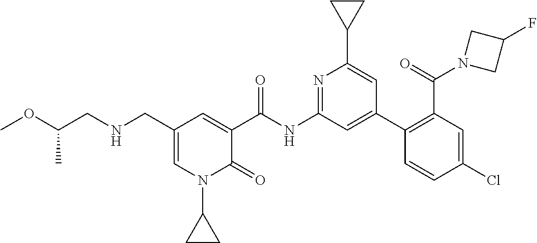 [SMILES: CO[C@@H](C)CNCc1cc(C(=O)Nc2cc(-c3ccc(Cl)cc3C(=O)N3CC(F)C3)cc(C3CC3)n2)c(=O)n(C2CC2)c1] | 608.2 | 608.3 |
| 249 | 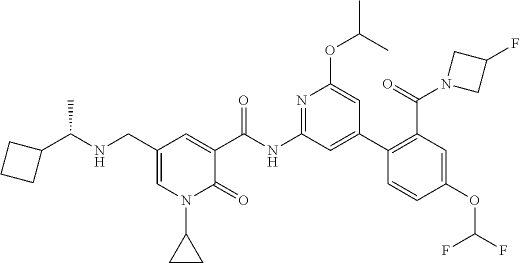 [SMILES: CC(C)Oc1cc(-c2ccc(OC(F)F)cc2C(=O)N2CC(F)C2)cc(NC(=O)c2cc(CN[C@@H](C)C3CCC3)cn(C3CC3)c2=O)n1] | 668.3 | 668.3 |
| 250 | 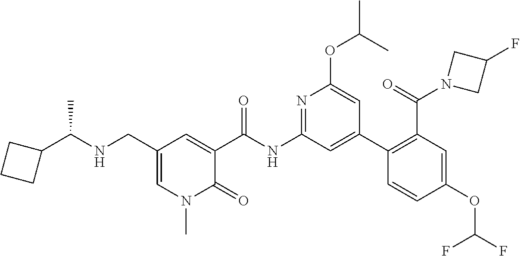 [SMILES: CC(C)Oc1cc(-c2ccc(OC(F)F)cc2C(=O)N2CC(F)C2)cc(NC(=O)c2cc(CN[C@@H](C)C3CCC3)cn(C)c2=O)n1] | 642.3 | 642.3 |
| 251 | 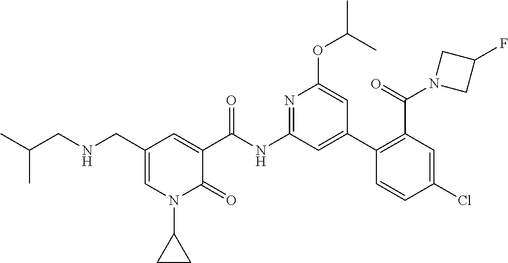 [SMILES: CC(C)CNCc1cc(C(=O)Nc2cc(-c3ccc(Cl)cc3C(=O)N3CC(F)C3)cc(OC(C)C)n2)c(=O)n(C2CC2)c1] | 610.3 | 610.3 |
| 252 | 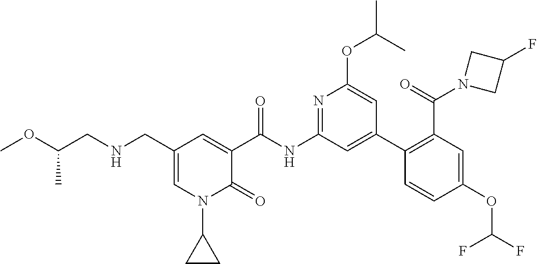 [SMILES: CO[C@@H](C)CNCc1cc(C(=O)Nc2cc(-c3ccc(OC(F)F)cc3C(=O)N3CC(F)C3)cc(OC(C)C)n2)c(=O)n(C2CC2)c1] | 658.3 | 658.3 |
| 253 | 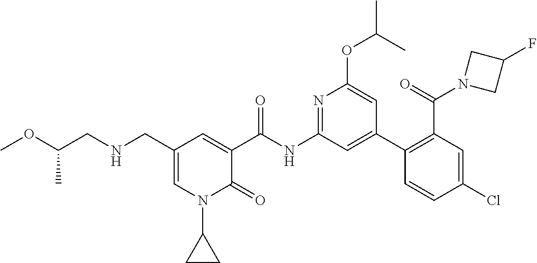 [SMILES: CO[C@@H](C)CNCc1cc(C(=O)Nc2cc(-c3ccc(Cl)cc3C(=O)N3CC(F)C3)cc(OC(C)C)n2)c(=O)n(C2CC2)c1] | 626.2 | 626.3 |
| 254 | 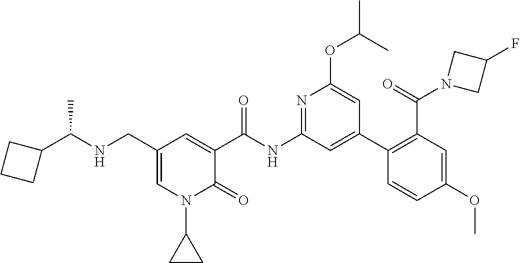 [SMILES: COc1ccc(-c2cc(NC(=O)c3cc(CN[C@@H](C)C4CCC4)cn(C4CC4)c3=O)nc(OC(C)C)c2)c(C(=O)N2CC(F)C2)c1] | 632.3 | 632.3 |
| 255 | 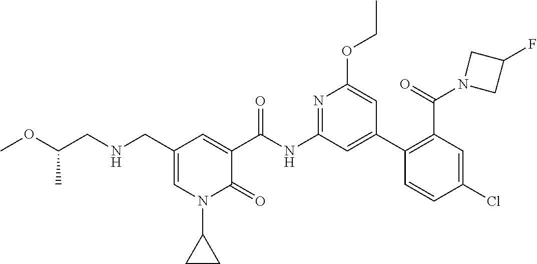 [SMILES: CCOc1cc(-c2ccc(Cl)cc2C(=O)N2CC(F)C2)cc(NC(=O)c2cc(CNC[C@H](C)OC)cn(C3CC3)c2=O)n1] | 612.2 | 612.3 |
| 256 | 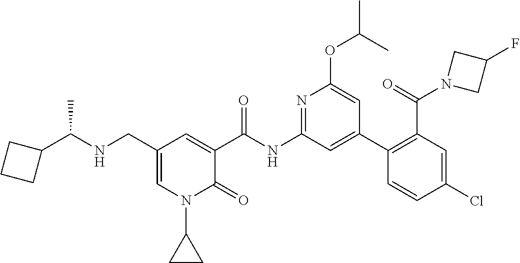 [SMILES: CC(C)Oc1cc(-c2ccc(Cl)cc2C(=O)N2CC(F)C2)cc(NC(=O)c2cc(CN[C@@H](C)C3CCC3)cn(C3CC3)c2=O)n1] | 636.3 | 636.3 |
| 257 | 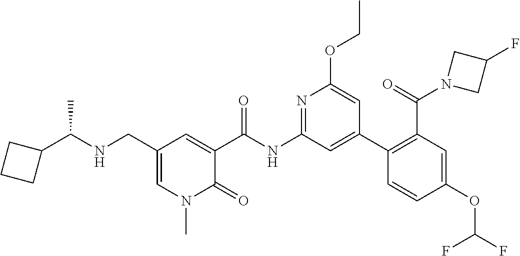 [SMILES: CCOc1cc(-c2ccc(OC(F)F)cc2C(=O)N2CC(F)C2)cc(NC(=O)c2cc(CN[C@@H](C)C3CCC3)cn(C)c2=O)n1] | 628.3 | 628.3 |
| 258 | 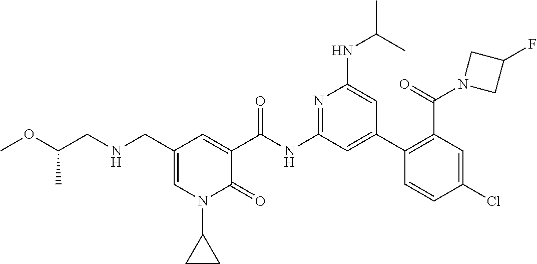 [SMILES: CO[C@@H](C)CNCc1cc(C(=O)Nc2cc(-c3ccc(Cl)cc3C(=O)N3CC(F)C3)cc(NC(C)C)n2)c(=O)n(C2CC2)c1] | 625.3 | 625.3 |
| 259 | 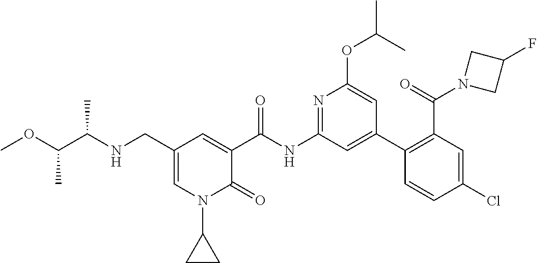 [SMILES: CO[C@@H](C)[C@H](C)NCc1cc(C(=O)Nc2cc(-c3ccc(Cl)cc3C(=O)N3CC(F)C3)cc(OC(C)C)n2)c(=O)n(C2CC2)c1] | 640.3 | 640.3 |
| 260 | 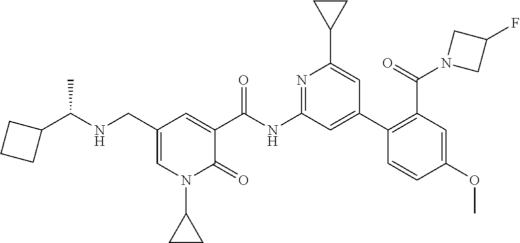 [SMILES: COc1ccc(-c2cc(NC(=O)c3cc(CN[C@@H](C)C4CCC4)cn(C4CC4)c3=O)nc(C3CC3)c2)c(C(=O)N2CC(F)C2)c1] | 614.3 | 614.4 |
| 261 | 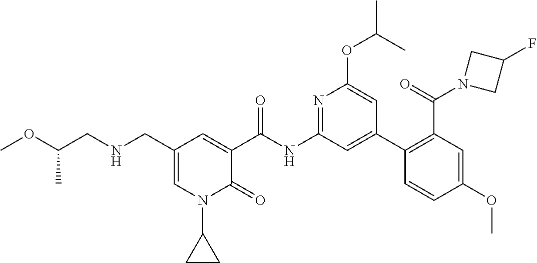 [SMILES: COc1ccc(-c2cc(NC(=O)c3cc(CNC[C@H](C)OC)cn(C4CC4)c3=O)nc(OC(C)C)c2)c(C(=O)N2CC(F)C2)c1] | 622.3 | 622.3 |
| 262 | 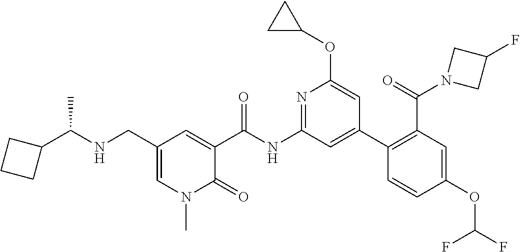 [SMILES: C[C@H](NCc1cc(C(=O)Nc2cc(-c3ccc(OC(F)F)cc3C(=O)N3CC(F)C3)cc(OC3CC3)n2)c(=O)n(C)c1)C1CCC1] | 640.3 | 640.3 |
| 263 | 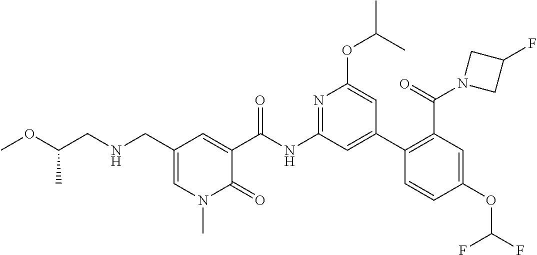 [SMILES: CO[C@@H](C)CNCc1cc(C(=O)Nc2cc(-c3ccc(OC(F)F)cc3C(=O)N3CC(F)C3)cc(OC(C)C)n2)c(=O)n(C)c1] | 632.3 | 632.3 |
| 264 | 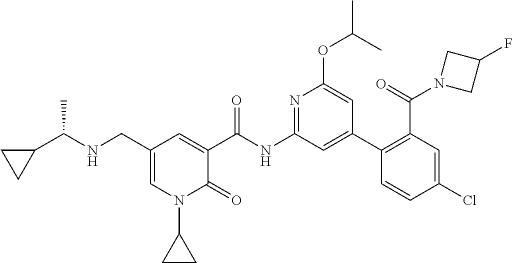 [SMILES: CC(C)Oc1cc(-c2ccc(Cl)cc2C(=O)N2CC(F)C2)cc(NC(=O)c2cc(CN[C@@H](C)C3CC3)cn(C3CC3)c2=O)n1] | 622.3 | 622.3 |
| 265 | 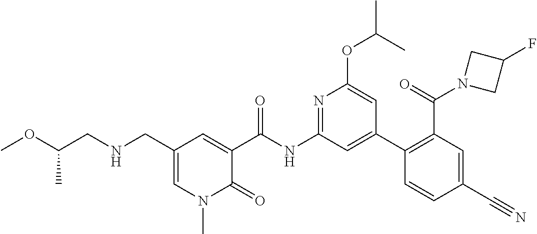 [SMILES: CO[C@@H](C)CNCc1cc(C(=O)Nc2cc(-c3ccc(C#N)cc3C(=O)N3CC(F)C3)cc(OC(C)C)n2)c(=O)n(C)c1] | 591.3 | 591.3 |
| 266 | 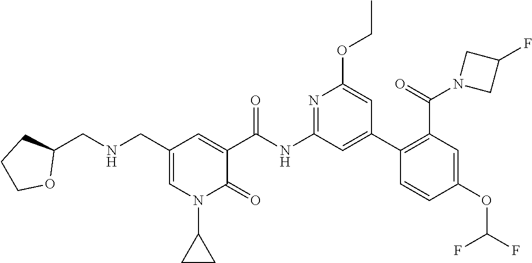 [SMILES: CCOc1cc(-c2ccc(OC(F)F)cc2C(=O)N2CC(F)C2)cc(NC(=O)c2cc(CNC[C@@H]3CCCO3)cn(C3CC3)c2=O)n1] | 656.3 | 656.3 |
| 267 | 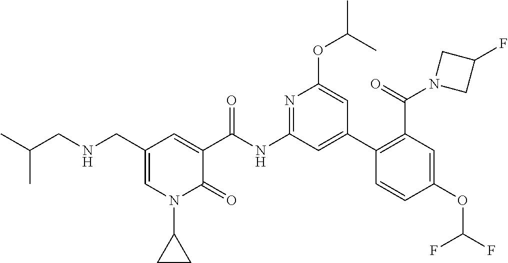 [SMILES: CC(C)CNCc1cc(C(=O)Nc2cc(-c3ccc(OC(F)F)cc3C(=O)N3CC(F)C3)cc(OC(C)C)n2)c(=O)n(C2CC2)c1] | 642.3 | 642.3 |
| 268 | 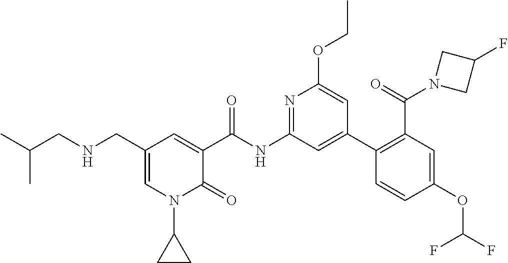 [SMILES: CCOc1cc(-c2ccc(OC(F)F)cc2C(=O)N2CC(F)C2)cc(NC(=O)c2cc(CNCC(C)C)cn(C3CC3)c2=O)n1] | 628.3 | 628.3 |
| 269 | 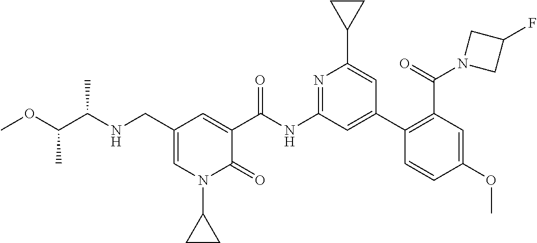 [SMILES: COc1ccc(-c2cc(NC(=O)c3cc(CN[C@@H](C)[C@H](C)OC)cn(C4CC4)c3=O)nc(C3CC3)c2)c(C(=O)N2CC(F)C2)c1] | 618.3 | 618.2 |
| 270 | 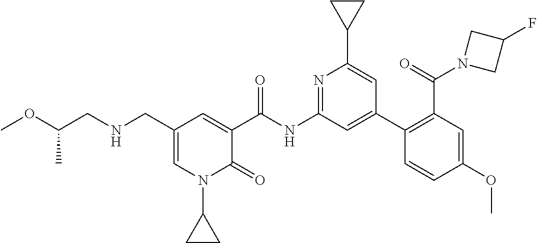 [SMILES: COc1ccc(-c2cc(NC(=O)c3cc(CNC[C@H](C)OC)cn(C4CC4)c3=O)nc(C3CC3)c2)c(C(=O)N2CC(F)C2)c1] | 604.3 | 604.2 |
| 271 | 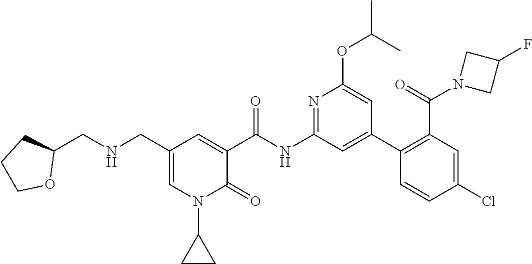 [SMILES: CC(C)Oc1cc(-c2ccc(Cl)cc2C(=O)N2CC(F)C2)cc(NC(=O)c2cc(CNC[C@@H]3CCCO3)cn(C3CC3)c2=O)n1] | 638.2 | 638.3 |
| 272 | 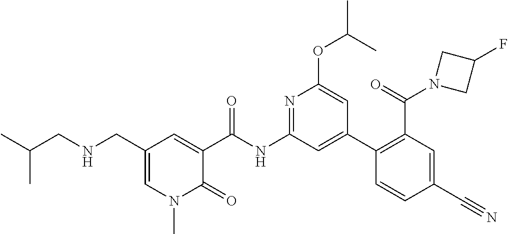 [SMILES: CC(C)CNCc1cc(C(=O)Nc2cc(-c3ccc(C#N)cc3C(=O)N3CC(F)C3)cc(OC(C)C)n2)c(=O)n(C)c1] | 575.3 | 575.3 |
| 273 | 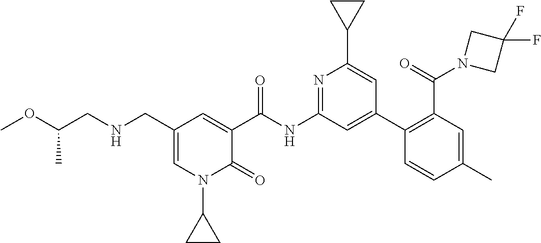 [SMILES: CO[C@@H](C)CNCc1cc(C(=O)Nc2cc(-c3ccc(C)cc3C(=O)N3CC(F)(F)C3)cc(C3CC3)n2)c(=O)n(C2CC2)c1] | 606.3 | 606.3 |
| 274 | 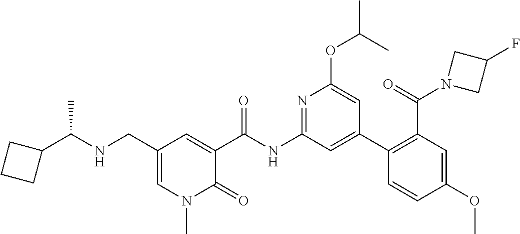 [SMILES: COc1ccc(-c2cc(NC(=O)c3cc(CN[C@@H](C)C4CCC4)cn(C)c3=O)nc(OC(C)C)c2)c(C(=O)N2CC(F)C2)c1] | 606.3 | 606.3 |
| 275 | 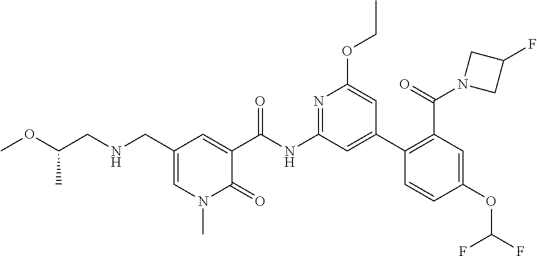 [SMILES: CCOc1cc(-c2ccc(OC(F)F)cc2C(=O)N2CC(F)C2)cc(NC(=O)c2cc(CNC[C@H](C)OC)cn(C)c2=O)n1] | 618.2 | 618.3 |
| 276 | 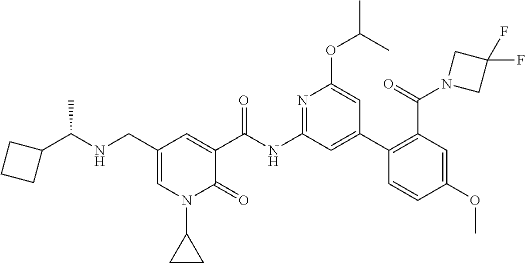 [SMILES: COc1ccc(-c2cc(NC(=O)c3cc(CN[C@@H](C)C4CCC4)cn(C4CC4)c3=O)nc(OC(C)C)c2)c(C(=O)N2CC(F)(F)C2)c1] | 650.3 | 650.3 |
| 277 | 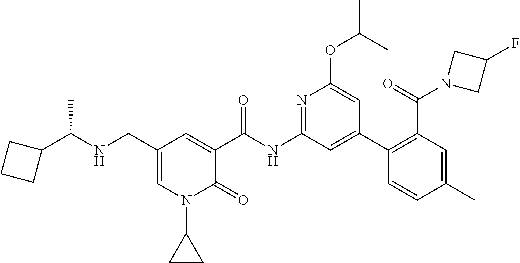 [SMILES: Cc1ccc(-c2cc(NC(=O)c3cc(CN[C@@H](C)C4CCC4)cn(C4CC4)c3=O)nc(OC(C)C)c2)c(C(=O)N2CC(F)C2)c1] | 616.3 | 616.3 |
| 278 | 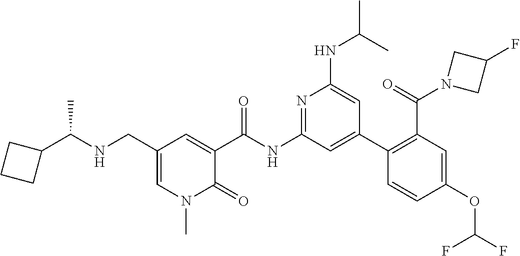 [SMILES: CC(C)Nc1cc(-c2ccc(OC(F)F)cc2C(=O)N2CC(F)C2)cc(NC(=O)c2cc(CN[C@@H](C)C3CCC3)cn(C)c2=O)n1] | 641.3 | 641.3 |
| 279 | 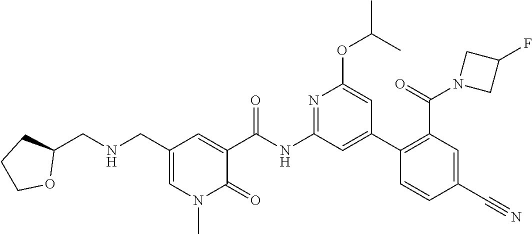 [SMILES: CC(C)Oc1cc(-c2ccc(C#N)cc2C(=O)N2CC(F)C2)cc(NC(=O)c2cc(CNC[C@@H]3CCCO3)cn(C)c2=O)n1] | 603.3 | 603.3 |
| 280 | 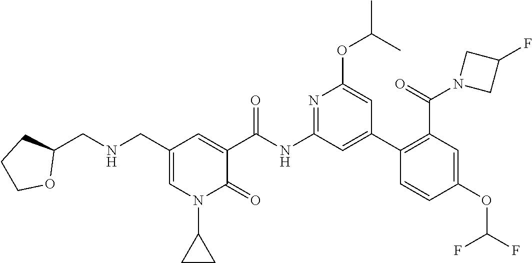 [SMILES: CC(C)Oc1cc(-c2ccc(OC(F)F)cc2C(=O)N2CC(F)C2)cc(NC(=O)c2cc(CNC[C@@H]3CCCO3)cn(C3CC3)c2=O)n1] | 670.3 | 670.3 |
| 281 | 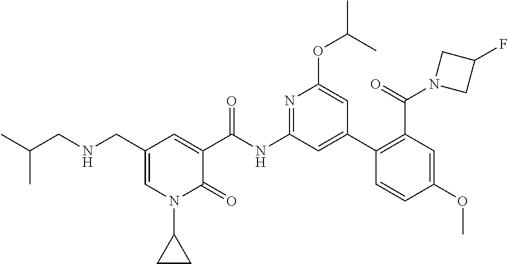 [SMILES: COc1ccc(-c2cc(NC(=O)c3cc(CNCC(C)C)cn(C4CC4)c3=O)nc(OC(C)C)c2)c(C(=O)N2CC(F)C2)c1] | 606.3 | 606.3 |
| 282 | 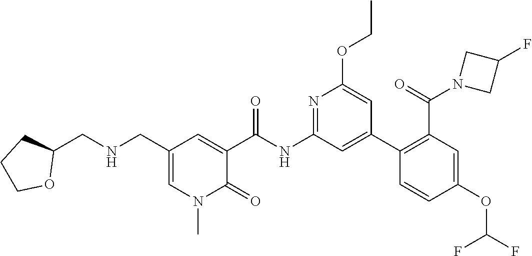 [SMILES: CCOc1cc(-c2ccc(OC(F)F)cc2C(=O)N2CC(F)C2)cc(NC(=O)c2cc(CNC[C@@H]3CCCO3)cn(C)c2=O)n1] | 630.2 | 630.3 |
| 283 | 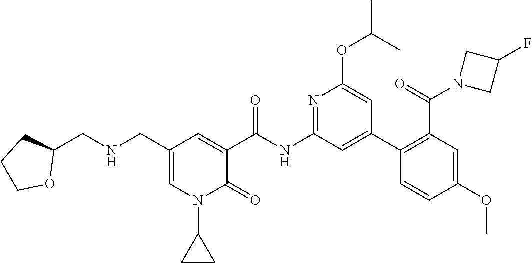 [SMILES: COc1ccc(-c2cc(NC(=O)c3cc(CNC[C@@H]4CCCO4)cn(C4CC4)c3=O)nc(OC(C)C)c2)c(C(=O)N2CC(F)C2)c1] | 634.3 | 634.3 |
| 284 | 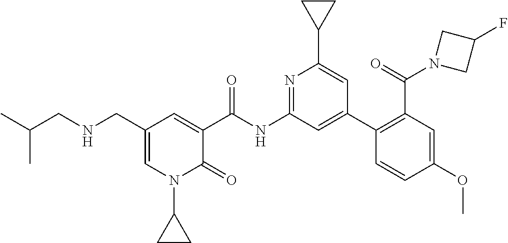 [SMILES: COc1ccc(-c2cc(NC(=O)c3cc(CNCC(C)C)cn(C4CC4)c3=O)nc(C3CC3)c2)c(C(=O)N2CC(F)C2)c1] | 588.3 | 588.3 |
| 285 | 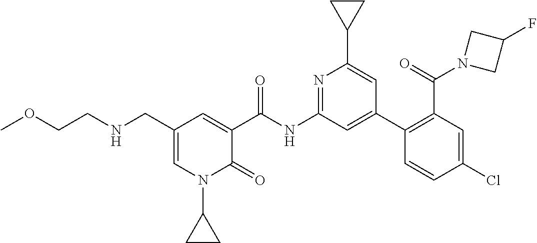 [SMILES: COCCNCc1cc(C(=O)Nc2cc(-c3ccc(Cl)cc3C(=O)N3CC(F)C3)cc(C3CC3)n2)c(=O)n(C2CC2)c1] | 594.2 | 594.2 |
| 286 | 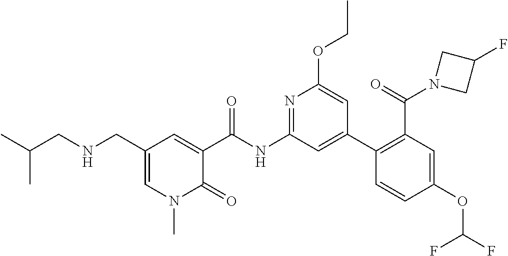 [SMILES: CCOc1cc(-c2ccc(OC(F)F)cc2C(=O)N2CC(F)C2)cc(NC(=O)c2cc(CNCC(C)C)cn(C)c2=O)n1] | 602.3 | 602.3 |
| 287 | 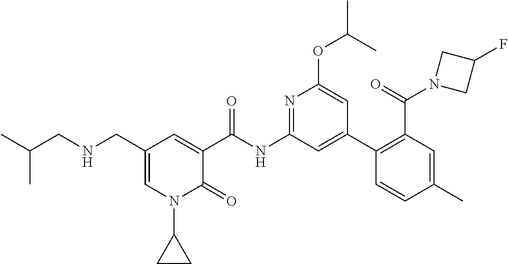 [SMILES: Cc1ccc(-c2cc(NC(=O)c3cc(CNCC(C)C)cn(C4CC4)c3=O)nc(OC(C)C)c2)c(C(=O)N2CC(F)C2)c1] | 590.3 | 590.3 |
| 288 | 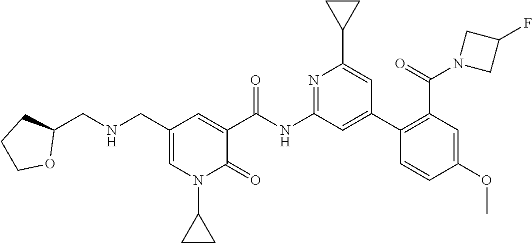 [SMILES: COc1ccc(-c2cc(NC(=O)c3cc(CNC[C@@H]4CCCO4)cn(C4CC4)c3=O)nc(C3CC3)c2)c(C(=O)N2CC(F)C2)c1] | 616.3 | 616.3 |
| 289 | 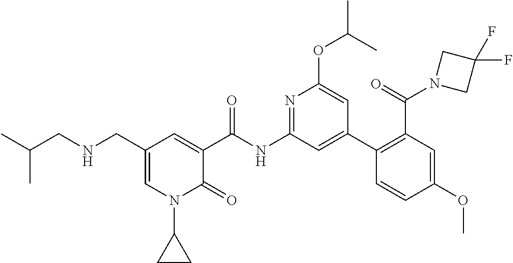 [SMILES: COc1ccc(-c2cc(NC(=O)c3cc(CNCC(C)C)cn(C4CC4)c3=O)nc(OC(C)C)c2)c(C(=O)N2CC(F)(F)C2)c1] | 624.3 | 624.3 |
| 290 | 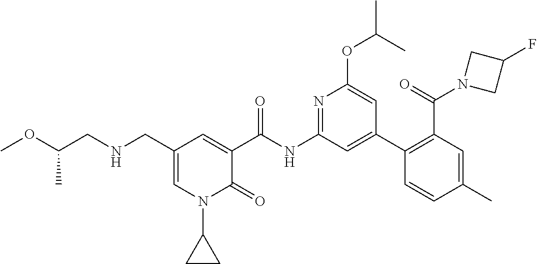 [SMILES: CO[C@@H](C)CNCc1cc(C(=O)Nc2cc(-c3ccc(C)cc3C(=O)N3CC(F)C3)cc(OC(C)C)n2)c(=O)n(C2CC2)c1] | 606.3 | 606.3 |
| 291 | 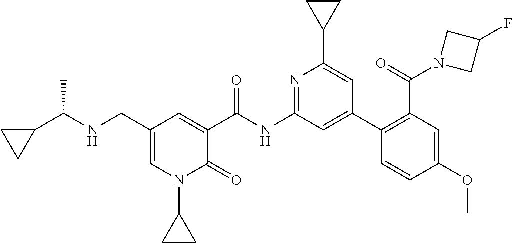 [SMILES: COc1ccc(-c2cc(NC(=O)c3cc(CN[C@@H](C)C4CC4)cn(C4CC4)c3=O)nc(C3CC3)c2)c(C(=O)N2CC(F)C2)c1] | 600.3 | 600.2 |
| 292 | 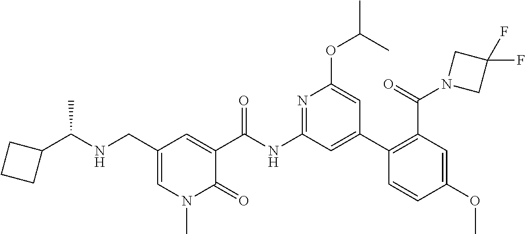 [SMILES: COc1ccc(-c2cc(NC(=O)c3cc(CN[C@@H](C)C4CCC4)cn(C)c3=O)nc(OC(C)C)c2)c(C(=O)N2CC(F)(F)C2)c1] | 624.3 | 624.3 |
| 293 | 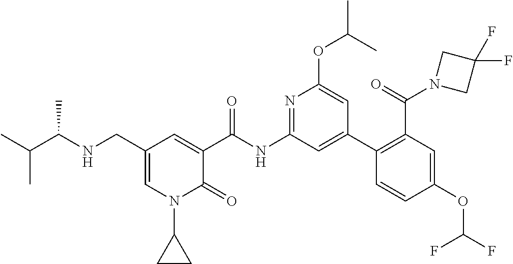 [SMILES: CC(C)Oc1cc(-c2ccc(OC(F)F)cc2C(=O)N2CC(F)(F)C2)cc(NC(=O)c2cc(CN[C@@H](C)C(C)C)cn(C3CC3)c2=O)n1] | 674.3 | 674.3 |
| 294 | 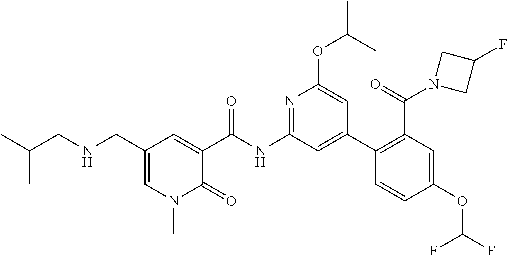 [SMILES: CC(C)CNCc1cc(C(=O)Nc2cc(-c3ccc(OC(F)F)cc3C(=O)N3CC(F)C3)cc(OC(C)C)n2)c(=O)n(C)c1] | 616.3 | 616.3 |
| 295 | 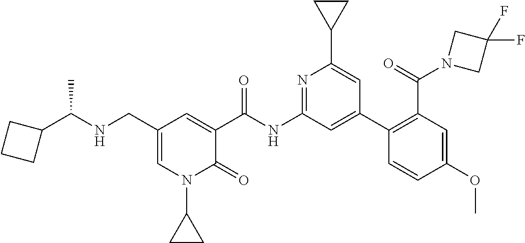 [SMILES: COc1ccc(-c2cc(NC(=O)c3cc(CN[C@@H](C)C4CCC4)cn(C4CC4)c3=O)nc(C3CC3)c2)c(C(=O)N2CC(F)(F)C2)c1] | 632.3 | 632.3 |
| 296 | 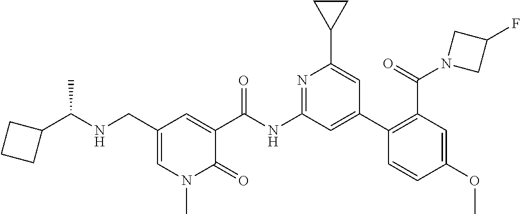 [SMILES: COc1ccc(-c2cc(NC(=O)c3cc(CN[C@@H](C)C4CCC4)cn(C)c3=O)nc(C3CC3)c2)c(C(=O)N2CC(F)C2)c1] | 588.3 | 588.3 |
| 297 | 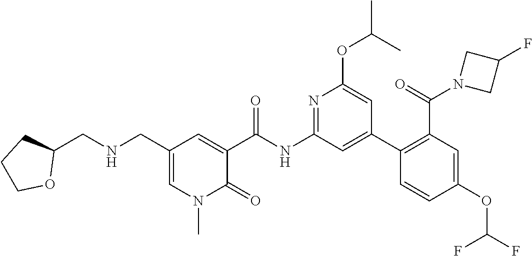 [SMILES: CC(C)Oc1cc(-c2ccc(OC(F)F)cc2C(=O)N2CC(F)C2)cc(NC(=O)c2cc(CNC[C@@H]3CCCO3)cn(C)c2=O)n1] | 644.3 | 644.3 |
| 298 | 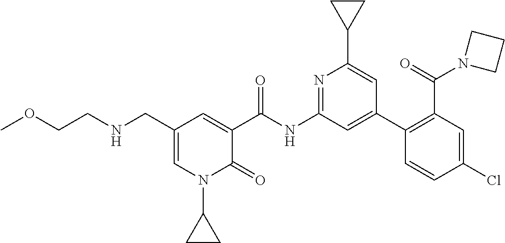 [SMILES: COCCNCc1cc(C(=O)Nc2cc(-c3ccc(Cl)cc3C(=O)N3CCC3)cc(C3CC3)n2)c(=O)n(C2CC2)c1] | 576.2 | 576.2 |
| 299 | 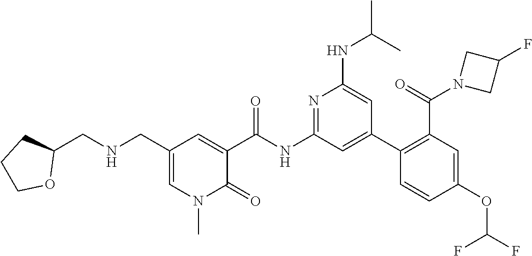 [SMILES: CC(C)Nc1cc(-c2ccc(OC(F)F)cc2C(=O)N2CC(F)C2)cc(NC(=O)c2cc(CNC[C@@H]3CCCO3)cn(C)c2=O)n1] | 643.3 | 643.3 |
| 300 | 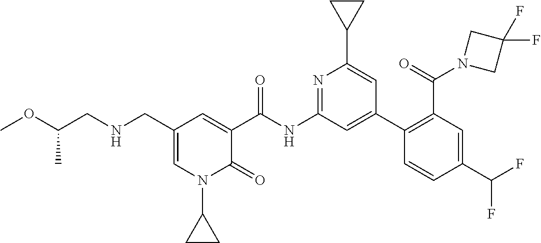 [SMILES: CO[C@@H](C)CNCc1cc(C(=O)Nc2cc(-c3ccc(C(F)F)cc3C(=O)N3CC(F)(F)C3)cc(C3CC3)n2)c(=O)n(C2CC2)c1] | 642.3 | 642.3 |
| 301 | 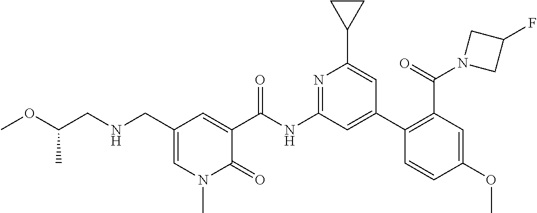 [SMILES: COc1ccc(-c2cc(NC(=O)c3cc(CNC[C@H](C)OC)cn(C)c3=O)nc(C3CC3)c2)c(C(=O)N2CC(F)C2)c1] | 578.3 | 578.3 |
| 302 | 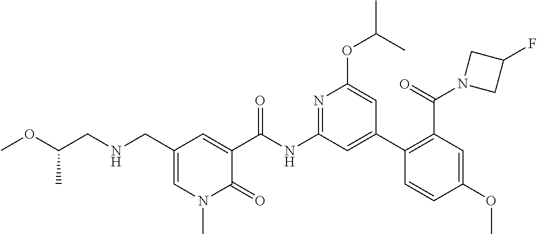 [SMILES: COc1ccc(-c2cc(NC(=O)c3cc(CNC[C@H](C)OC)cn(C)c3=O)nc(OC(C)C)c2)c(C(=O)N2CC(F)C2)c1] | 596.3 | 596.3 |
| 303 | 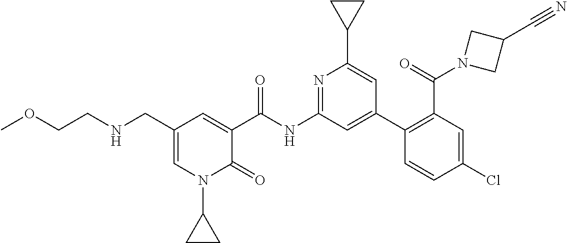 [SMILES: COCCNCc1cc(C(=O)Nc2cc(-c3ccc(Cl)cc3C(=O)N3CC(C#N)C3)cc(C3CC3)n2)c(=O)n(C2CC2)c1] | 601.2 | 601.2 |
| 304 | 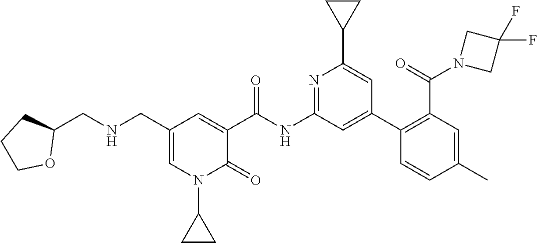 [SMILES: Cc1ccc(-c2cc(NC(=O)c3cc(CNC[C@@H]4CCCO4)cn(C4CC4)c3=O)nc(C3CC3)c2)c(C(=O)N2CC(F)(F)C2)c1] | 618.3 | 618.3 |
| 305 | 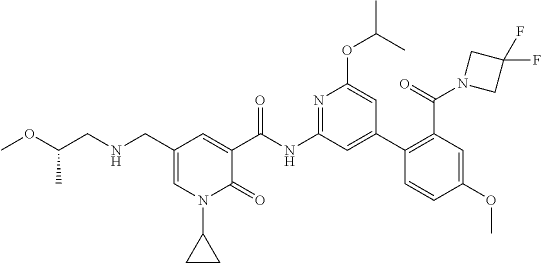 [SMILES: COc1ccc(-c2cc(NC(=O)c3cc(CNC[C@H](C)OC)cn(C4CC4)c3=O)nc(OC(C)C)c2)c(C(=O)N2CC(F)(F)C2)c1] | 640.3 | 640.3 |
| 306 | 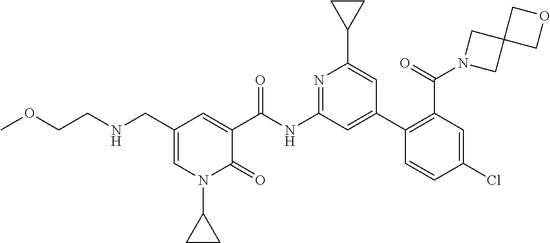 [SMILES: COCCNCc1cc(C(=O)Nc2cc(-c3ccc(Cl)cc3C(=O)N3CC4(COC4)C3)cc(C3CC3)n2)c(=O)n(C2CC2)c1] | 618.2 | 618.3 |
| 307 | 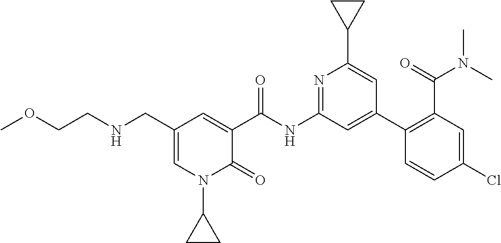 | 564.2 | 564.2 |
| 308 | 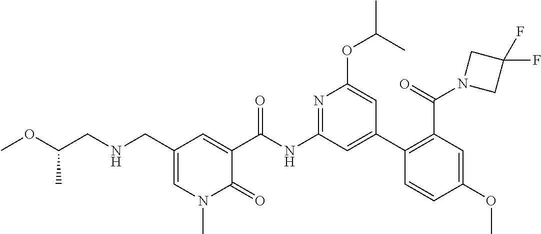 [SMILES: COc1ccc(-c2cc(NC(=O)c3cc(CNC[C@H](C)OC)cn(C)c3=O)nc(OC(C)C)c2)c(C(=O)N2CC(F)(F)C2)c1] | 614.3 | 614.3 |
| 309 | 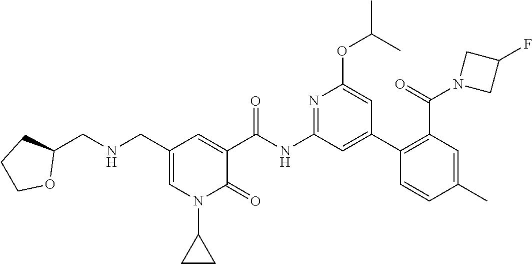 [SMILES: Cc1ccc(-c2cc(NC(=O)c3cc(CNC[C@@H]4CCCO4)cn(C4CC4)c3=O)nc(OC(C)C)c2)c(C(=O)N2CC(F)C2)c1] | 618.3 | 618.3 |
| 310 | 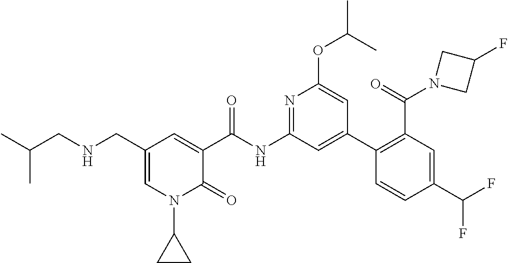 [SMILES: CC(C)CNCc1cc(C(=O)Nc2cc(-c3ccc(C(F)F)cc3C(=O)N3CC(F)C3)cc(OC(C)C)n2)c(=O)n(C2CC2)c1] | 626.3 | 626.3 |
| 311 | 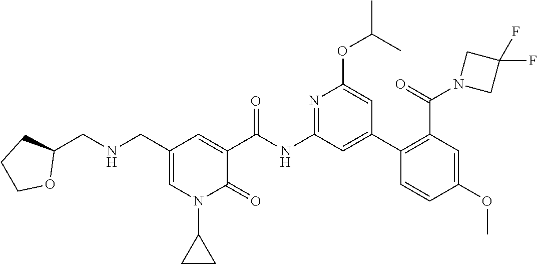 [SMILES: COc1ccc(-c2cc(NC(=O)c3cc(CNC[C@@H]4CCCO4)cn(C4CC4)c3=O)nc(OC(C)C)c2)c(C(=O)N2CC(F)(F)C2)c1] | 652.3 | 652.3 |
| 312 | 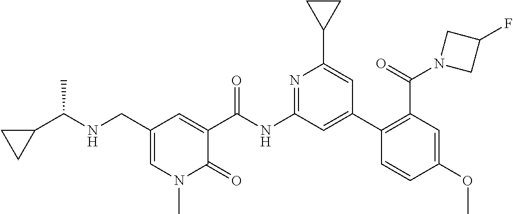 [SMILES: COc1ccc(-c2cc(NC(=O)c3cc(CN[C@@H](C)C4CC4)cn(C)c3=O)nc(C3CC3)c2)c(C(=O)N2CC(F)C2)c1] | 574.3 | 574.3 |
| 313 | 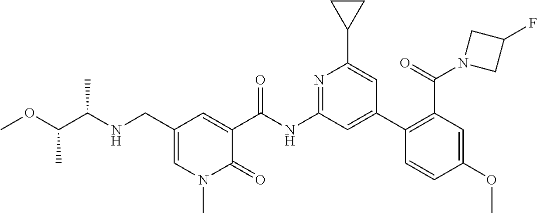 [SMILES: COc1ccc(-c2cc(NC(=O)c3cc(CN[C@@H](C)[C@H](C)OC)cn(C)c3=O)nc(C3CC3)c2)c(C(=O)N2CC(F)C2)c1] | 592.3 | 592.2 |
| 314 | 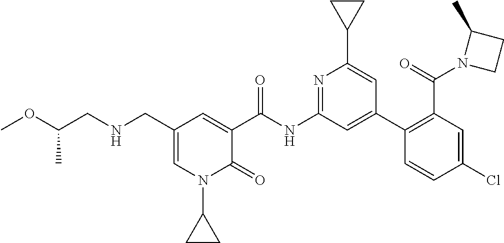 [SMILES: CO[C@@H](C)CNCc1cc(C(=O)Nc2cc(-c3ccc(Cl)cc3C(=O)N3CC[C@@H]3C)cc(C3CC3)n2)c(=O)n(C2CC2)c1] | 604.3 | 604.3 |
| 315 | 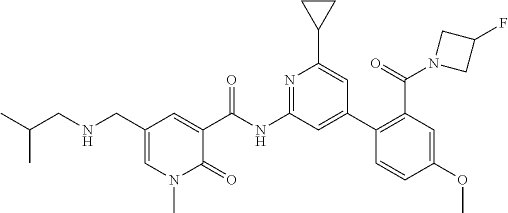 [SMILES: COc1ccc(-c2cc(NC(=O)c3cc(CNCC(C)C)cn(C)c3=O)nc(C3CC3)c2)c(C(=O)N2CC(F)C2)c1] | 562.3 | 562.3 |
| 316 | 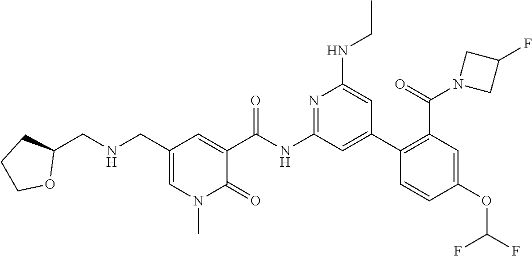 [SMILES: CCNc1cc(-c2ccc(OC(F)F)cc2C(=O)N2CC(F)C2)cc(NC(=O)c2cc(CNC[C@@H]3CCCO3)cn(C)c2=O)n1] | 629.3 | 629.3 |
| 317 | 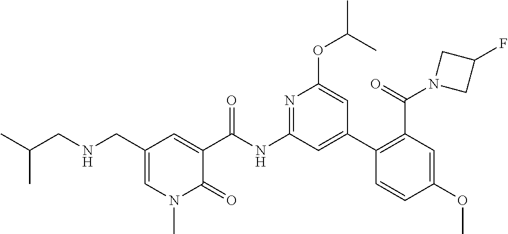 [SMILES: COc1ccc(-c2cc(NC(=O)c3cc(CNCC(C)C)cn(C)c3=O)nc(OC(C)C)c2)c(C(=O)N2CC(F)C2)c1] | 580.3 | 580.3 |
| 318 | 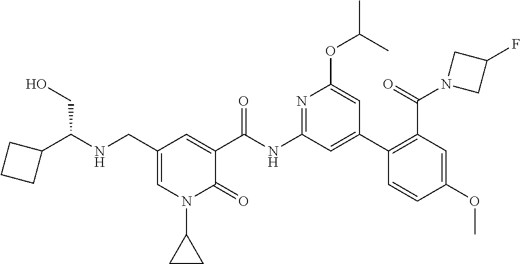 [SMILES: COc1ccc(-c2cc(NC(=O)c3cc(CN[C@@H](CO)C4CCC4)cn(C4CC4)c3=O)nc(OC(C)C)c2)c(C(=O)N2CC(F)C2)c1] | 648.3 | 648.3 |
| 319 | 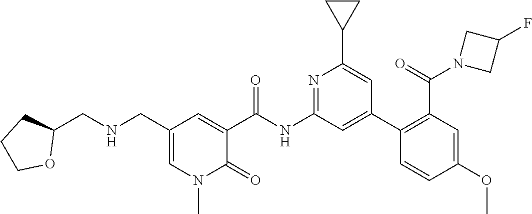 [SMILES: COc1ccc(-c2cc(NC(=O)c3cc(CNC[C@@H]4CCCO4)cn(C)c3=O)nc(C3CC3)c2)c(C(=O)N2CC(F)C2)c1] | 590.3 | 590.3 |
| 320 | 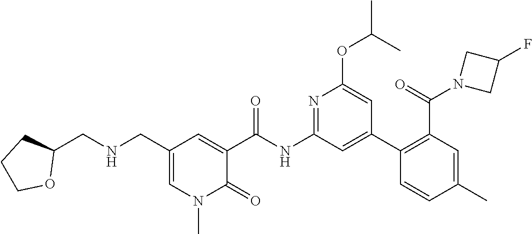 [SMILES: Cc1ccc(-c2cc(NC(=O)c3cc(CNC[C@@H]4CCCO4)cn(C)c3=O)nc(OC(C)C)c2)c(C(=O)N2CC(F)C2)c1] | 592.3 | 592.3 |
| 321 | 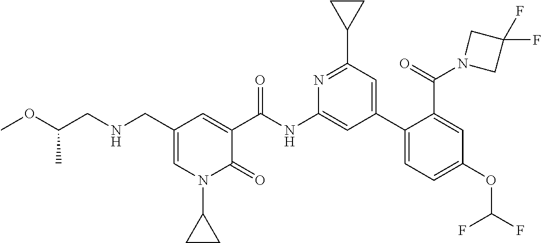 [SMILES: CO[C@@H](C)CNCc1cc(C(=O)Nc2cc(-c3ccc(OC(F)F)cc3C(=O)N3CC(F)(F)C3)cc(C3CC3)n2)c(=O)n(C2CC2)c1] | 658.3 | 658.2 |
| 322 | 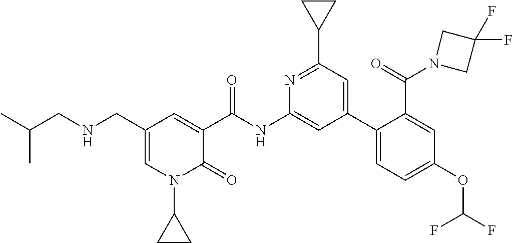 [SMILES: CC(C)CNCc1cc(C(=O)Nc2cc(-c3ccc(OC(F)F)cc3C(=O)N3CC(F)(F)C3)cc(C3CC3)n2)c(=O)n(C2CC2)c1] | 642.3 | 642.2 |
| 323 | 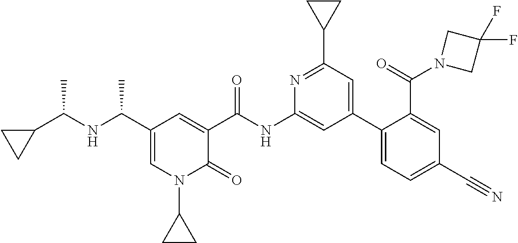 [SMILES: C[C@H](N[C@H](C)c1cc(C(=O)Nc2cc(-c3ccc(C#N)cc3C(=O)N3CC(F)(F)C3)cc(C3CC3)n2)c(=O)n(C2CC2)c1)C1CC1] | 627.3 | 627.2 |
| 324 | 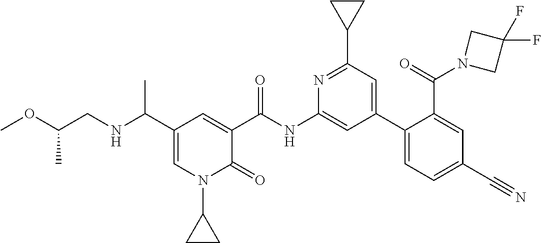 [SMILES: CO[C@@H](C)CNC(C)c1cc(C(=O)Nc2cc(-c3ccc(C#N)cc3C(=O)N3CC(F)(F)C3)cc(C3CC3)n2)c(=O)n(C2CC2)c1] | 631.3 | 631.2 |
| 325 | 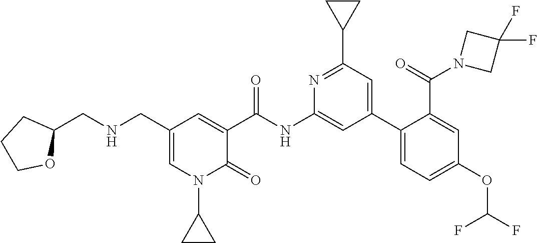 [SMILES: O=C(Nc1cc(-c2ccc(OC(F)F)cc2C(=O)N2CC(F)(F)C2)cc(C2CC2)n1)c1cc(CNC[C@@H]2CCCO2)cn(C2CC2)c1=O] | 670.3 | 670.2 |
| 326 | 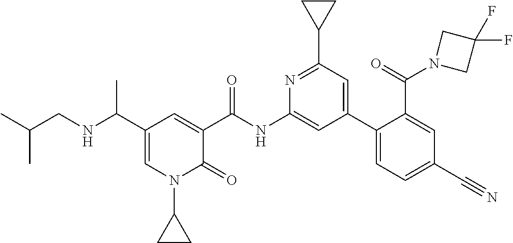 [SMILES: CC(C)CNC(C)c1cc(C(=O)Nc2cc(-c3ccc(C#N)cc3C(=O)N3CC(F)(F)C3)cc(C3CC3)n2)c(=O)n(C2CC2)c1] | 615.3 | 615.2 |
| 327 | 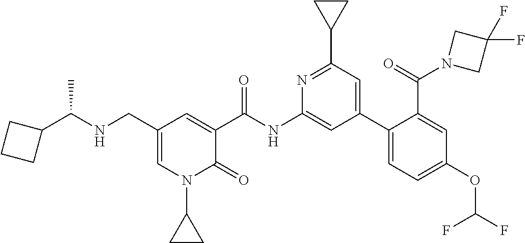 [SMILES: C[C@H](NCc1cc(C(=O)Nc2cc(-c3ccc(OC(F)F)cc3C(=O)N3CC(F)(F)C3)cc(C3CC3)n2)c(=O)n(C2CC2)c1)C1CCC1] | 668.3 | 668.2 |
| 328 | 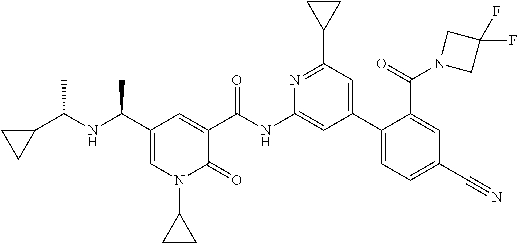 [SMILES: C[C@H](N[C@@H](C)C1CC1)c1cc(C(=O)Nc2cc(-c3ccc(C#N)cc3C(=O)N3CC(F)(F)C3)cc(C3CC3)n2)c(=O)n(C2CC2)c1] | 627.3 | 627.2 |
| 329 | 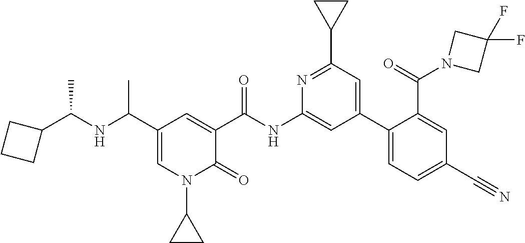 [SMILES: CC(N[C@@H](C)C1CCC1)c1cc(C(=O)Nc2cc(-c3ccc(C#N)cc3C(=O)N3CC(F)(F)C3)cc(C3CC3)n2)c(=O)n(C2CC2)c1] | 641.3 | 641.2 |
| 330 | 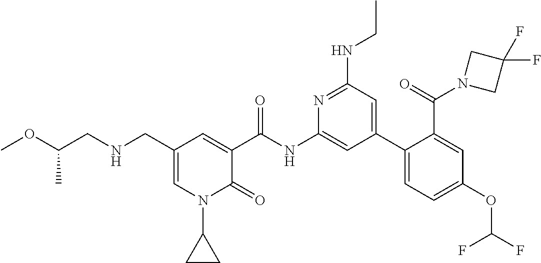 [SMILES: CCNc1cc(-c2ccc(OC(F)F)cc2C(=O)N2CC(F)(F)C2)cc(NC(=O)c2cc(CNC[C@H](C)OC)cn(C3CC3)c2=O)n1] | 661.3 | 661.2 |
| 331 | 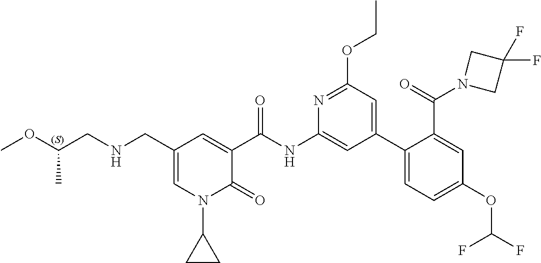 | 662.3 | 662.3 |
| 332 | 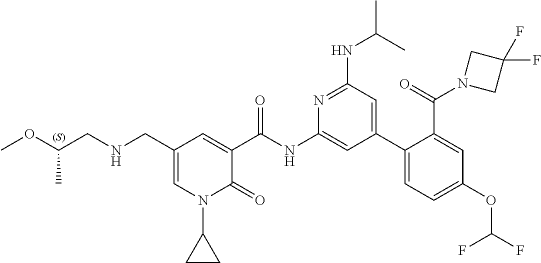 | 675.3 | 675.3 |
| 333 | 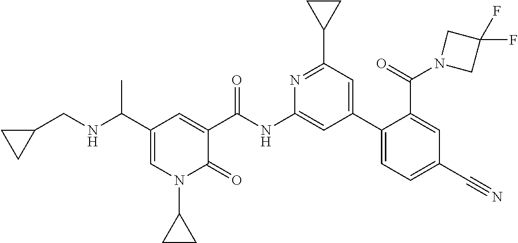 [SMILES: CC(NCC1CC1)c1cc(C(=O)Nc2cc(-c3ccc(C#N)cc3C(=O)N3CC(F)(F)C3)cc(C3CC3)n2)c(=O)n(C2CC2)c1] | 613.3 | 613.2 |
| 334 | 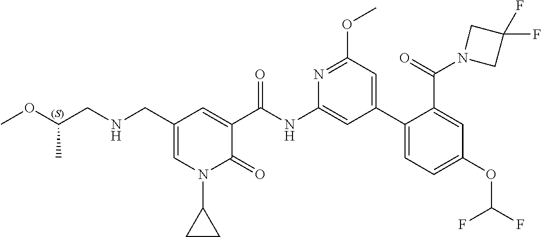 | 648.2 | 648.2 |
| 335 | 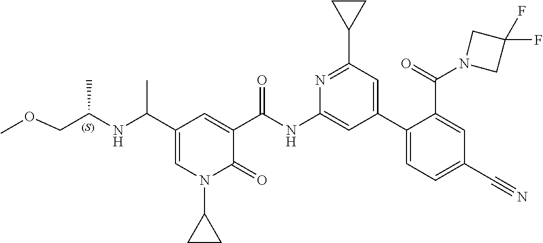 | 631.3 | 631.2 |
| 336 | 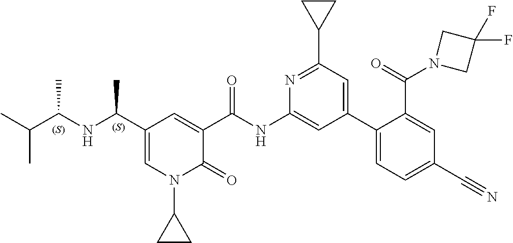 | 629.3 | 629.2 |
| 337 | 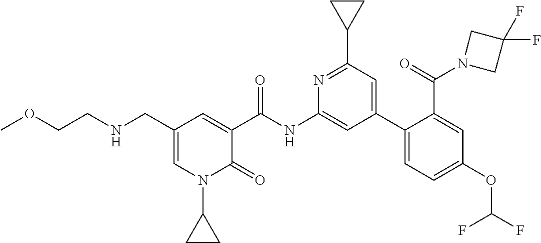 [SMILES: COCCNCc1cc(C(=O)Nc2cc(-c3ccc(OC(F)F)cc3C(=O)N3CC(F)(F)C3)cc(C3CC3)n2)c(=O)n(C2CC2)c1] | 644.2 | 644.2 |
| 338 | 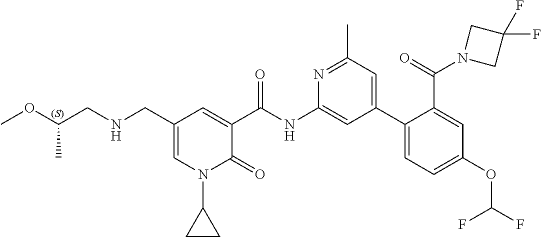 | 632.2 | 632.3 |
| 339 | 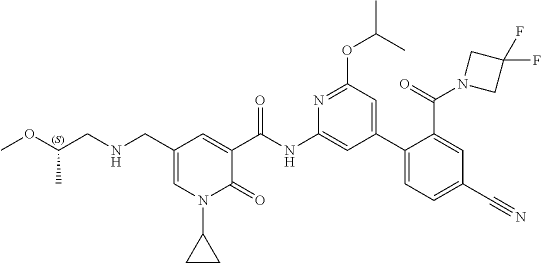 | 635.3 | 635.3 |
| 340 | 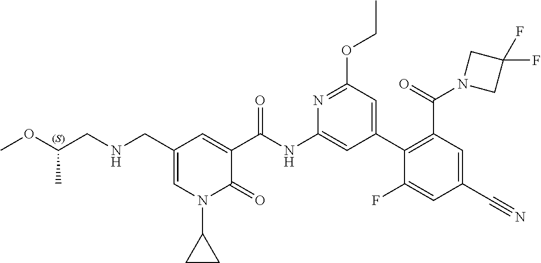 | 639.2 | 639.2 |
| 341 | 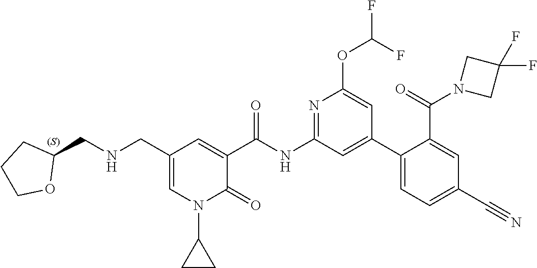 | 655.2 | 655.3 |
| 342 | 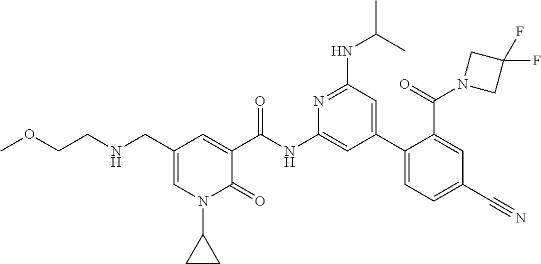 [SMILES: COCCNCc1cc(C(=O)Nc2cc(-c3ccc(C#N)cc3C(=O)N3CC(F)(F)C3)cc(NC(C)C)n2)c(=O)n(C2CC2)c1] | 620.3 | 620.3 |
| 343 | 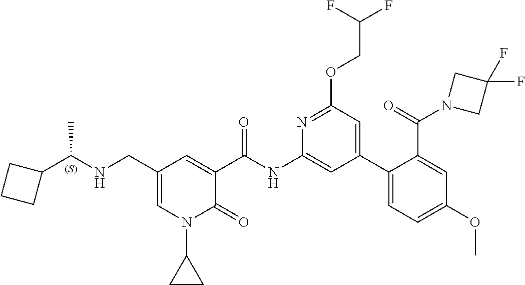 | 672.3 | 672.3 |
| 344 | 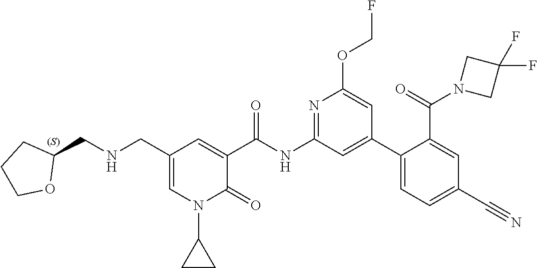 | 637.2 | 637.3 |
| 345 | 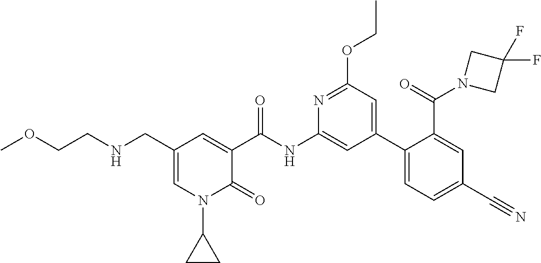 [SMILES: CCOc1cc(-c2ccc(C#N)cc2C(=O)N2CC(F)(F)C2)cc(NC(=O)c2cc(CNCCOC)cn(C3CC3)c2=O)n1] | 607.2 | 607.2 |
| 346 | 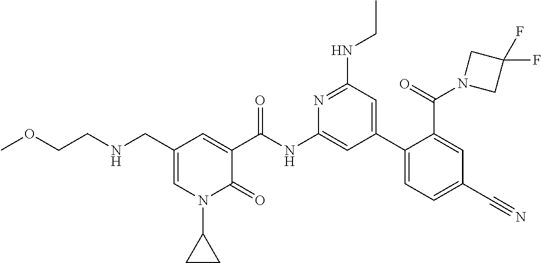 [SMILES: CCNc1cc(-c2ccc(C#N)cc2C(=O)N2CC(F)(F)C2)cc(NC(=O)c2cc(CNCCOC)cn(C3CC3)c2=O)n1] | 606.3 | 606.2 |
| 347 | 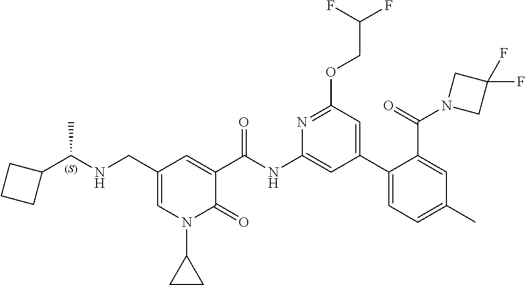 | 656.3 | 656.3 |
| 348 | 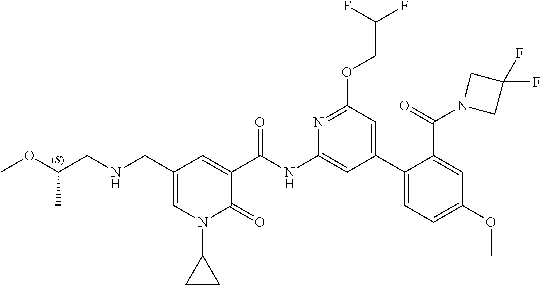 | 662.3 | 662.3 |
| 349 | 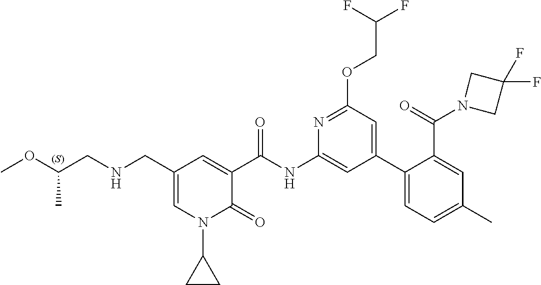 | 646.3 | 646.3 |
| 350 | 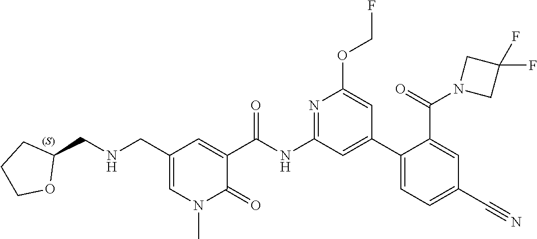 | 611.2 | 611.2 |
| 351 | 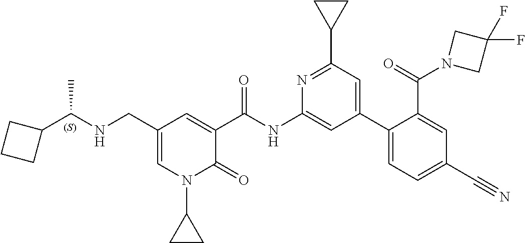 | 627.3 | 627.2 |
| 352 | 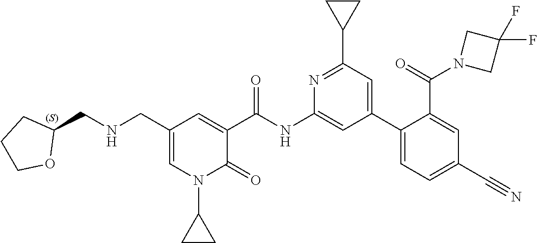 | 629.3 | 629.2 |
| 353 | 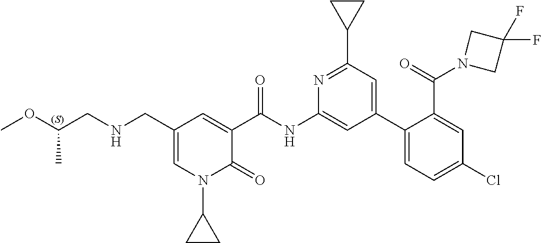 | 626.2 | 626.2 |
| 354 | 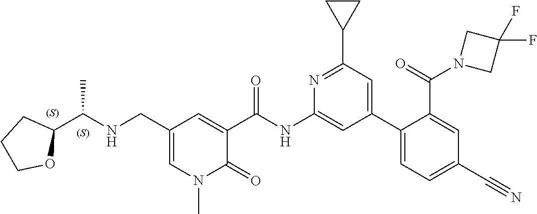 | 617.3 | 617.2 |
| 355 | 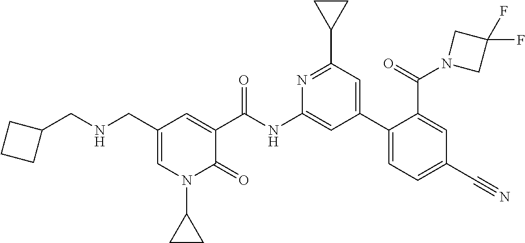 [SMILES: N#Cc1ccc(-c2cc(NC(=O)c3cc(CNCC4CCC4)cn(C4CC4)c3=O)nc(C3CC3)c2)c(C(=O)N2CC(F)(F)C2)c1] | 613.3 | 613.2 |
| 356 | 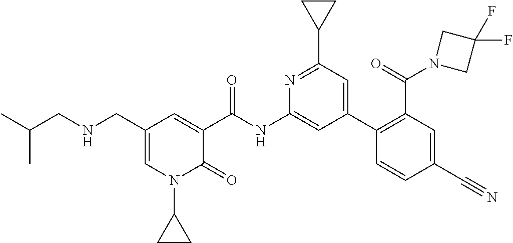 [SMILES: CC(C)CNCc1cc(C(=O)Nc2cc(-c3ccc(C#N)cc3C(=O)N3CC(F)(F)C3)cc(C3CC3)n2)c(=O)n(C2CC2)c1] | 601.3 | 601.2 |
| 357 | 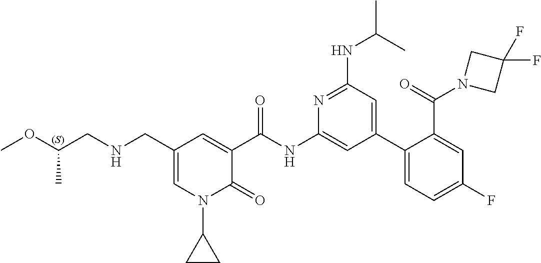 | 627.3 | 627.2 |
| 358 | 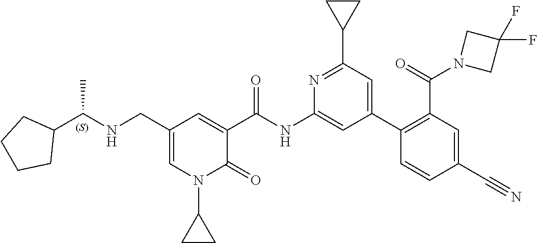 | 641.3 | 641.3 |
| 359 | 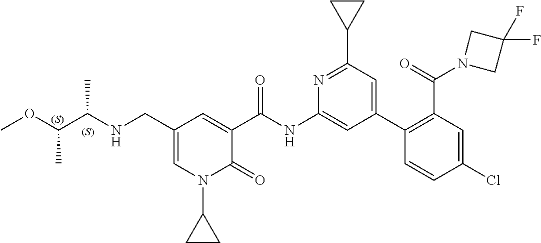 | 640.2 | 640.2 |
| 360 | 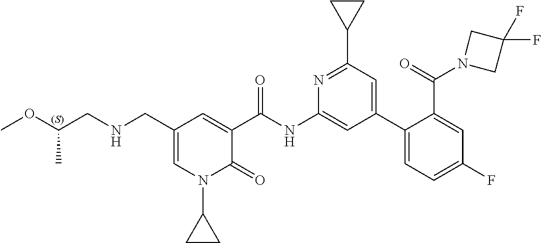 | 610.3 | 610.2 |
| 361 | 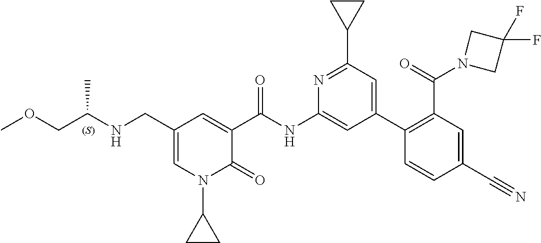 | 617.3 | 617.2 |
| 362 | 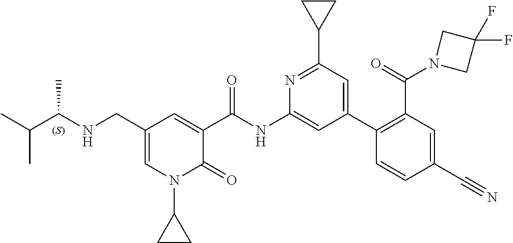 | 615.3 | 615.2 |
| 363 | 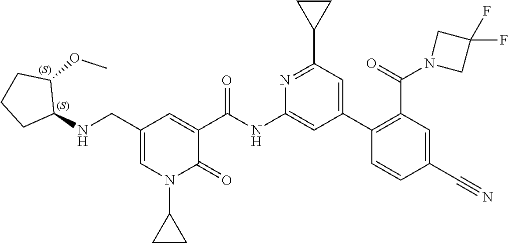 | 643.3 | 643.2 |
| 364 | 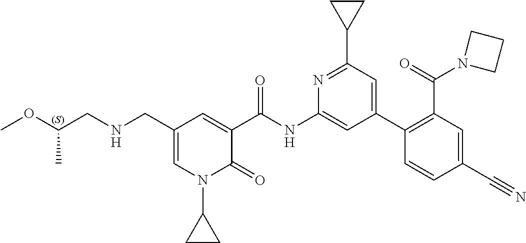 | 581.3 | 581.2 |
| 365 | 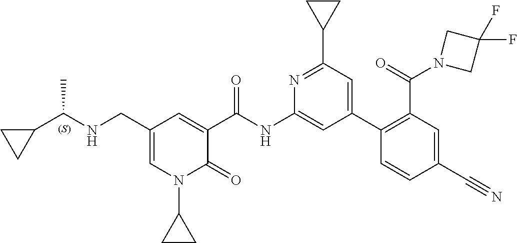 | 613.3 | 613.2 |
| 366 | 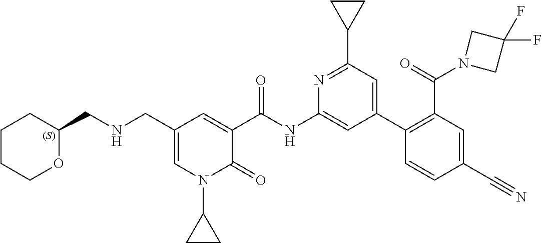 | 643.3 | 643.2 |
| 367 | 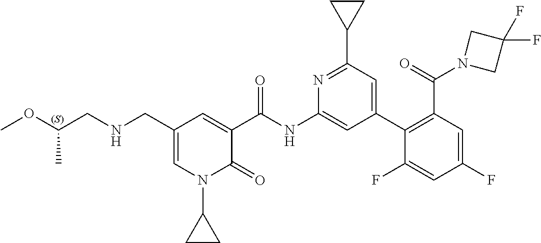 | 628.2 | 628.2 |
| 368 | 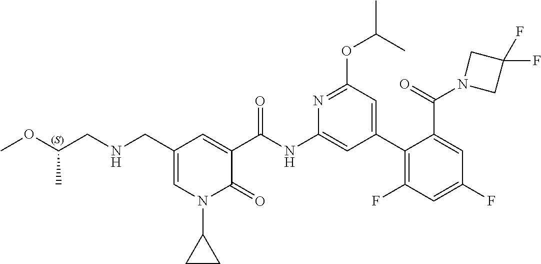 | 646.3 | 646.2 |
| 369 | 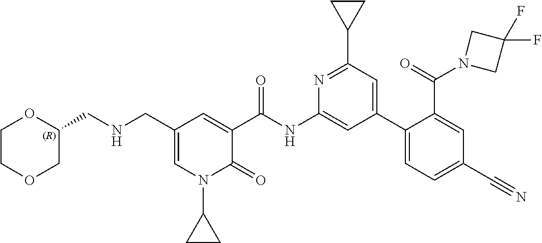 | 645.3 | 645.2 |
| 370 | 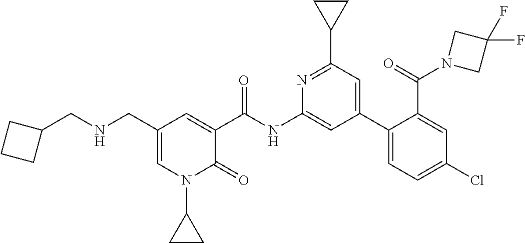 [SMILES: O=C(Nc1cc(-c2ccc(Cl)cc2C(=O)N2CC(F)(F)C2)cc(C2CC2)n1)c1cc(CNCC2CCC2)cn(C2CC2)c1=O] | 622.2 | 622.2 |
| 371 | 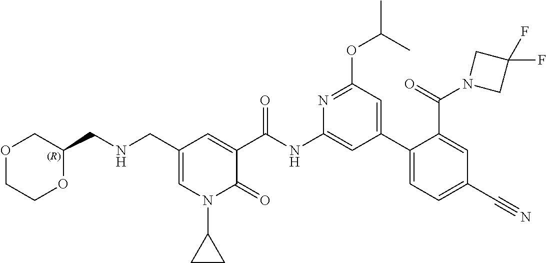 | 663.3 | 663.2 |
| 372 | 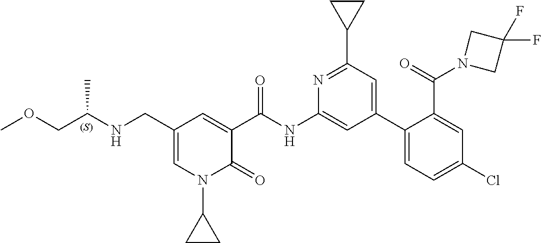 | 626.2 | 626.2 |
| 373 | 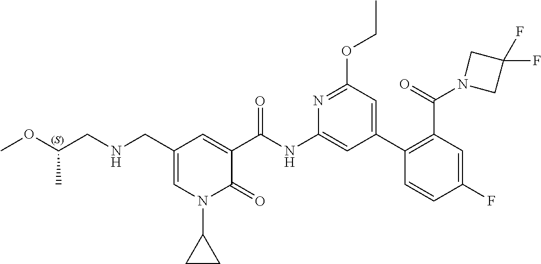 | 614.3 | 614.2 |
| 374 | 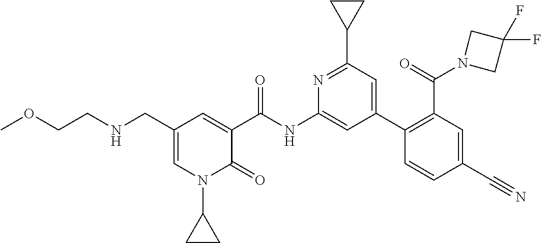 [SMILES: COCCNCc1cc(C(=O)Nc2cc(-c3ccc(C#N)cc3C(=O)N3CC(F)(F)C3)cc(C3CC3)n2)c(=O)n(C2CC2)c1] | 603.2 | 603.2 |
| 375 | 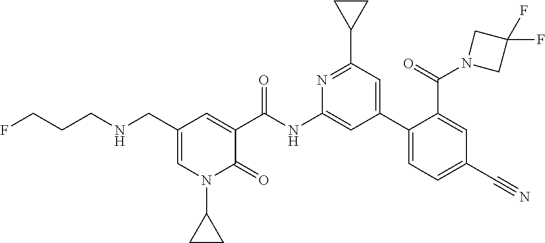 [SMILES: N#Cc1ccc(-c2cc(NC(=O)c3cc(CNCCCF)cn(C4CC4)c3=O)nc(C3CC3)c2)c(C(=O)N2CC(F)(F)C2)c1] | 605.2 | 605.2 |
| 376 | 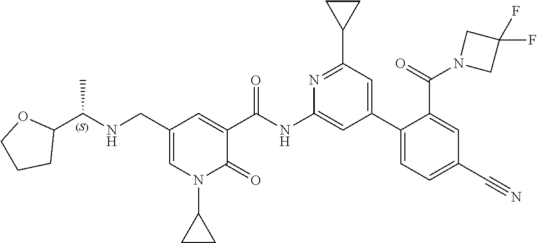 | 643.3 | 643.2 |
| 377 | 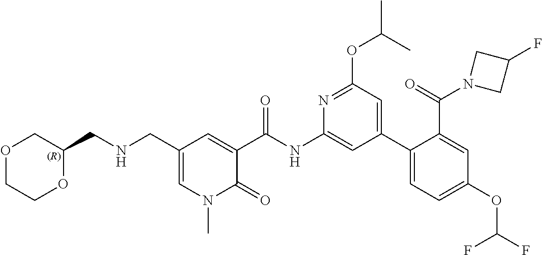 | 660.3 | 660.2 |
| 378 | 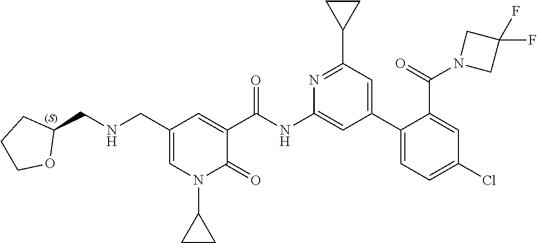 | 638.2 | 638.2 |
| 379 | 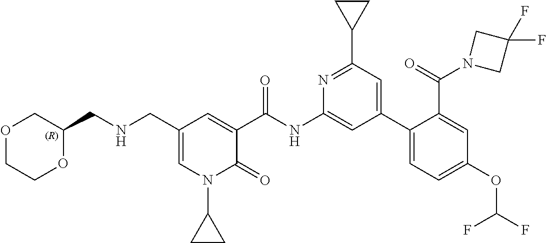 | 686.3 | 686.2 |
| 380 | 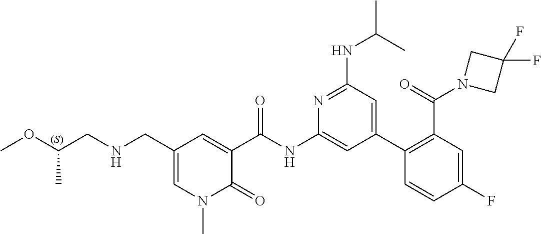 | 601.3 | 601.3 |
| 381 | 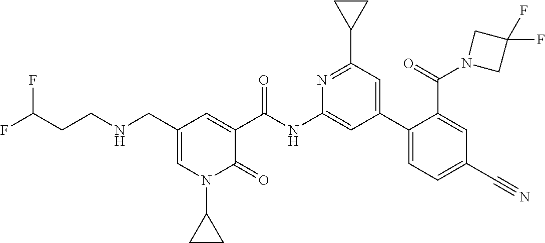 [SMILES: N#Cc1ccc(-c2cc(NC(=O)c3cc(CNCCC(F)F)cn(C4CC4)c3=O)nc(C3CC3)c2)c(C(=O)N2CC(F)(F)C2)c1] | 623.2 | 623.2 |
| 382 | 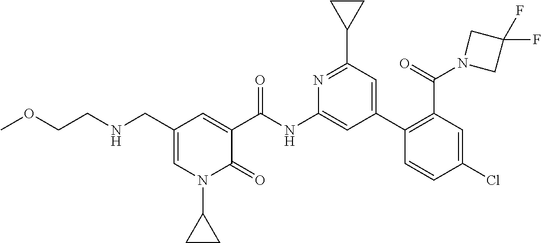 [SMILES: COCCNCc1cc(C(=O)Nc2cc(-c3ccc(Cl)cc3C(=O)N3CC(F)(F)C3)cc(C3CC3)n2)c(=O)n(C2CC2)c1] | 612.2 | 612.2 |
| 383 | 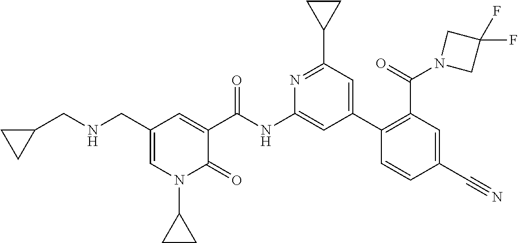 [SMILES: N#Cc1ccc(-c2cc(NC(=O)c3cc(CNCC4CC4)cn(C4CC4)c3=O)nc(C3CC3)c2)c(C(=O)N2CC(F)(F)C2)c1] | 599.3 | 599.2 |
| 384 | 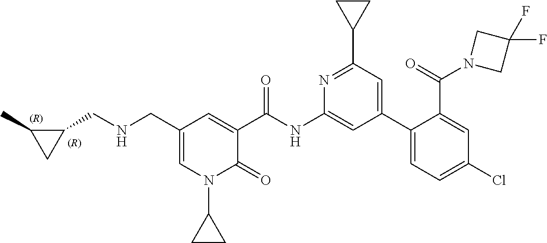 | 622.2 | 622.2 |
| 385 | 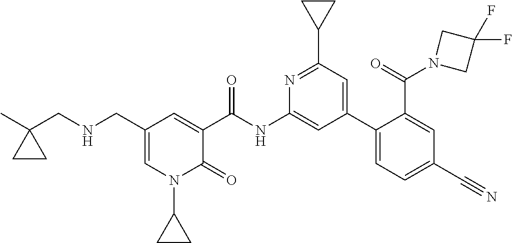 [SMILES: CC1(CNCc2cc(C(=O)Nc3cc(-c4ccc(C#N)cc4C(=O)N4CC(F)(F)C4)cc(C4CC4)n3)c(=O)n(C3CC3)c2)CC1] | 613.3 | 613.2 |
| 386 | 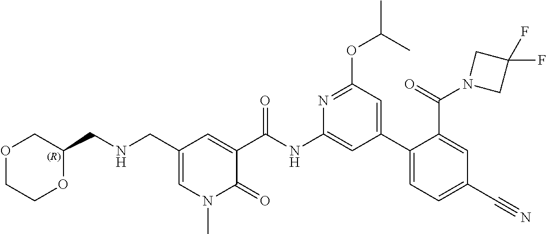 | 637.3 | 637.2 |
| 387 | 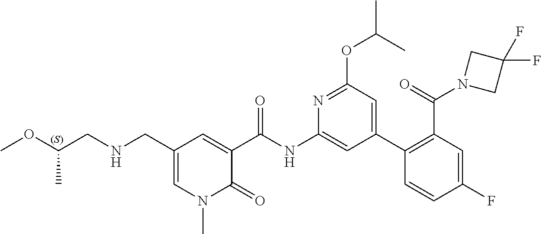 | 602.3 | 602.2 |
| 388 | 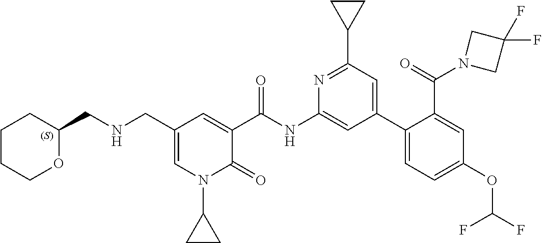 | 684.3 | 684.2 |
| 389 | 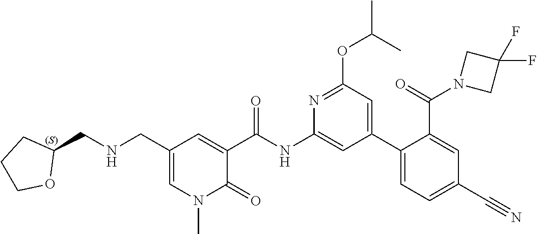 | 621.3 | 621.2 |
| 390 | 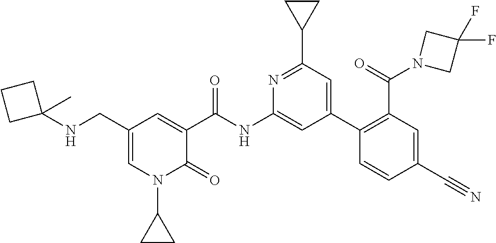 [SMILES: CC1(NCc2cc(C(=O)Nc3cc(-c4ccc(C#N)cc4C(=O)N4CC(F)(F)C4)cc(C4CC4)n3)c(=O)n(C3CC3)c2)CCC1] | 613.3 | 613.2 |
| 391 | 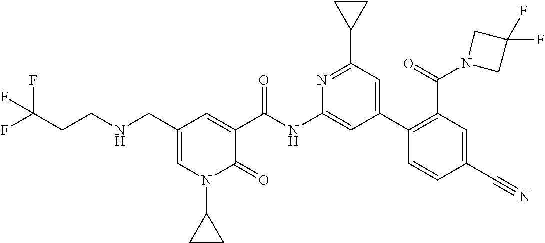 [SMILES: N#Cc1ccc(-c2cc(NC(=O)c3cc(CNCCC(F)(F)F)cn(C4CC4)c3=O)nc(C3CC3)c2)c(C(=O)N2CC(F)(F)C2)c1] | 641.2 | 641.2 |
| 392 | 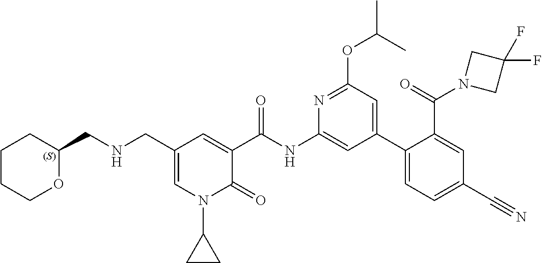 | 661.3 | 661.3 |
| 393 | 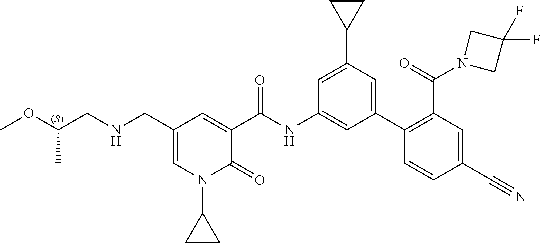 | 616.3 | 616.2 |
| 394 | 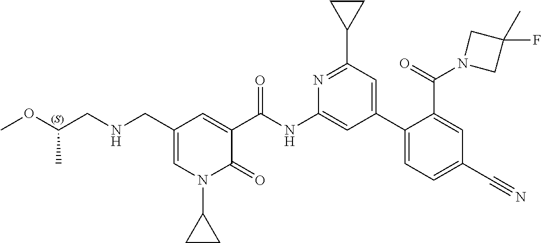 | 613.3 | 613.2 |
| 395 | 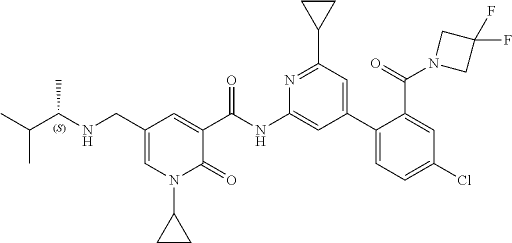 | 624.2 | 624.2 |
| 396 | 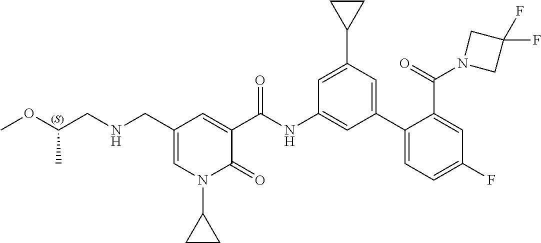 | 609.3 | 609.3 |
| 397 | 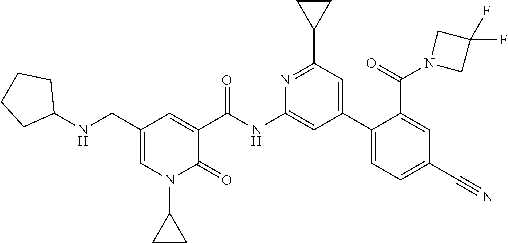 [SMILES: N#Cc1ccc(-c2cc(NC(=O)c3cc(CNC4CCCC4)cn(C4CC4)c3=O)nc(C3CC3)c2)c(C(=O)N2CC(F)(F)C2)c1] | 613.3 | 613.3 |
| 398 | 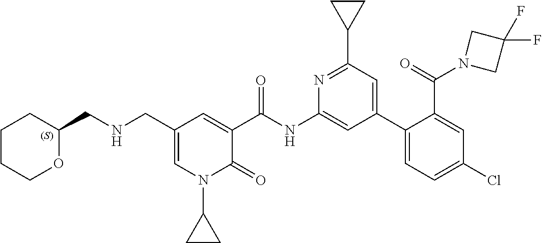 | 652.2 | 652.2 |
| 399 | 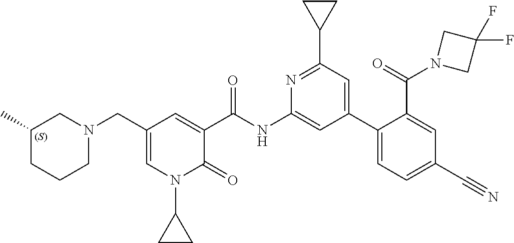 | 627.3 | 627.3 |
| 400 | 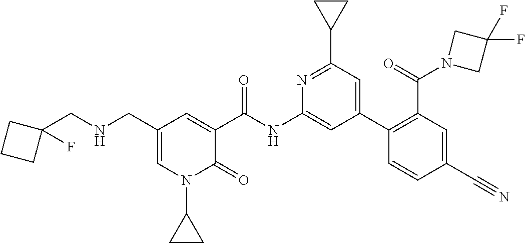 [SMILES: N#Cc1ccc(-c2cc(NC(=O)c3cc(CNCC4(F)CCC4)cn(C4CC4)c3=O)nc(C3CC3)c2)c(C(=O)N2CC(F)(F)C2)c1] | 631.3 | 631.2 |
| 401 | 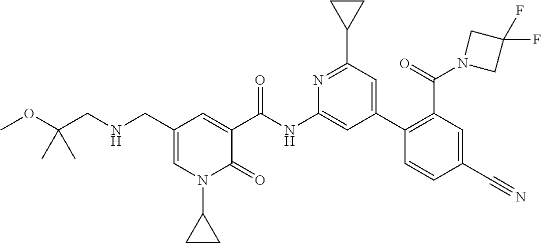 [SMILES: COC(C)(C)CNCc1cc(C(=O)Nc2cc(-c3ccc(C#N)cc3C(=O)N3CC(F)(F)C3)cc(C3CC3)n2)c(=O)n(C2CC2)c1] | 631.3 | 631.2 |
| 402 | 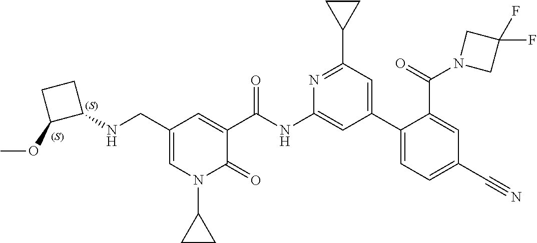 | 629.3 | 629.2 |
| 403 | 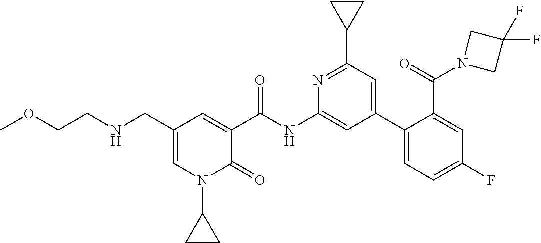 [SMILES: COCCNCc1cc(C(=O)Nc2cc(-c3ccc(F)cc3C(=O)N3CC(F)(F)C3)cc(C3CC3)n2)c(=O)n(C2CC2)c1] | 596.2 | 596.2 |
| 404 | 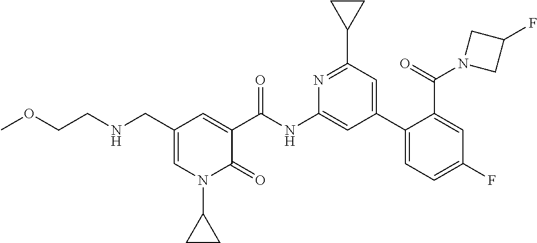 [SMILES: COCCNCc1cc(C(=O)Nc2cc(-c3ccc(F)cc3C(=O)N3CC(F)C3)cc(C3CC3)n2)c(=O)n(C2CC2)c1] | 578.3 | 578.2 |
| 405 | 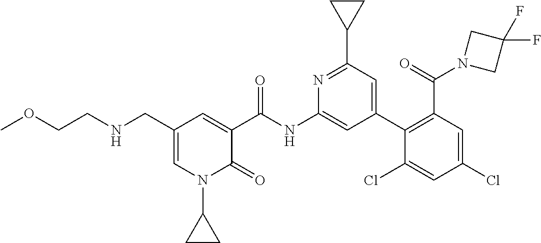 [SMILES: COCCNCc1cc(C(=O)Nc2cc(-c3c(Cl)cc(Cl)cc3C(=O)N3CC(F)(F)C3)cc(C3CC3)n2)c(=O)n(C2CC2)c1] | 646.2 | 646.2 |
| 406 | 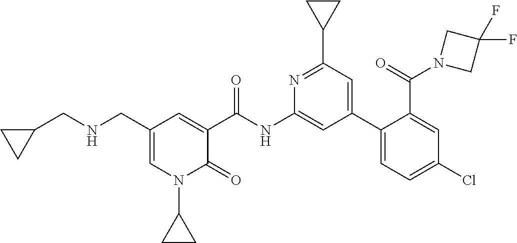 [SMILES: O=C(Nc1cc(-c2ccc(Cl)cc2C(=O)N2CC(F)(F)C2)cc(C2CC2)n1)c1cc(CNCC2CC2)cn(C2CC2)c1=O] | 608.2 | 608.2 |
| 407 | 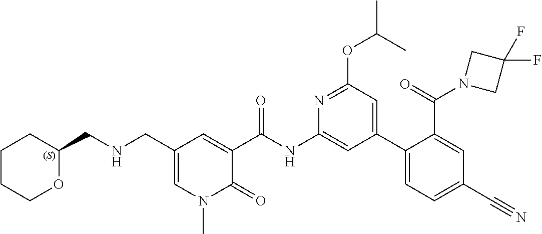 | 635.3 | 635.2 |
| 408 | 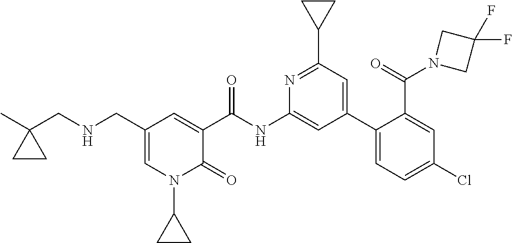 [SMILES: CC1(CNCc2cc(C(=O)Nc3cc(-c4ccc(Cl)cc4C(=O)N4CC(F)(F)C4)cc(C4CC4)n3)c(=O)n(C3CC3)c2)CC1] | 622.2 | 622.2 |
| 409 | 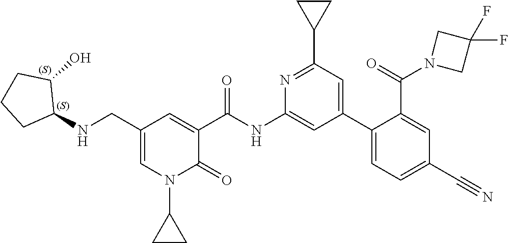 | 629.3 | 629.2 |
| 410 | 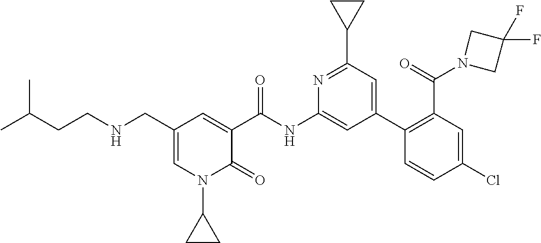 [SMILES: CC(C)CCNCc1cc(C(=O)Nc2cc(-c3ccc(Cl)cc3C(=O)N3CC(F)(F)C3)cc(C3CC3)n2)c(=O)n(C2CC2)c1] | 624.2 | 624.2 |
| 411 | 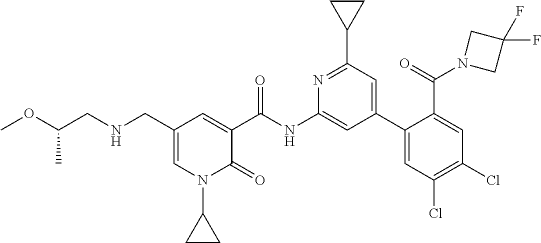 [SMILES: CO[C@@H](C)CNCc1cc(C(=O)Nc2cc(-c3cc(Cl)c(Cl)cc3C(=O)N3CC(F)(F)C3)cc(C3CC3)n2)c(=O)n(C2CC2)c1] | 660.2 | 660.2 |
| 412 | 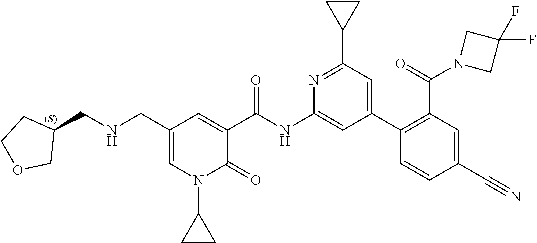 | 629.3 | 629.2 |

### Biological Assay Examples

The affinity with which compounds of the present disclosure bind to Cbl-b was assessed using probe displacement homologous time resolved fluorescence (HTRF) assays. The assays used a BODIPY™ conjugated probe (Example 54 from WO 2020264398) and biotinylated Cbl-b. The assays were performed in assay buffer consisting of 20 mM Hepes, 150 mM NaCl, 0.01% Triton X-100, 0.5 mM TCEP, 0.01% BSA. On the day of the assay, a 20 point, 1:2 master serial dilution of each compound was prepared in DMSO to span a final concentration range of 10 M to 0 nM. Two hundred nanoliters of diluted compound was added to each well of a 384-well plate. Fifteen microliters of biotinylated- Cbl-b resuspended in assay buffer were added to each well and the plate incubated for 60 minutes at room temperature prior to addition of 5 μL of BODIPY™ probe in assay buffer. Final assay conditions included 0.4 nM of Cbl-b and 150 nM of BODIPY™ probe. After a further 15 minutes of incubation at room temperature, 5 μl of Streptavidin-Terbium cryptate reagent at 5-fold final concentration was added to each well of the 384-well plate. Binding of a probe to Cbl-b results in an increase of HTRF signal. HTRF signal was measured by Envision plate reader, while competition of a compound of the present disclosure with probe results in a decrease of signal. Percentage maximum activity in each test well was calculated based on DMSO (maximum activity, 0% displacement) and no protein control wells (baseline activity, 100% displacement). Binding affinity was determined from a dose response curve fitted using a standard four parameter fit equation. See Table 7 for data for compounds (Cbl-b Binding (IC₅₀)).

Certain compounds were also evaluated in an IL-2 secretion assay. On the day of the assay, a 16 point, 1:2 master serial dilution of each compound was prepared in Opti-MEM to span a final concentration range of 10 μM to 300 pM. Assays were set up in CORNING® tissue culture-treated 384-well microplates containing 60 nL of each dilution. Jurkat cells grown in RPMI-1640 supplemented with 10% FBS, 1% Glutamax, and 1% Pen/Strep were collected, resuspended in Opti-MEM. 50,000 cells/well and added to the compound plates. After a short spin (1200 rpm for 1 min), the plates were incubated at 37° C. for 1 hour. The cells were activated by adding 15 μL of IMMUNOCULT™ Human CD3/CD28 T Cell Activator (STEMCELL Technologies) diluted in Opti-MEM. After 24 h of incubation at 37° C., aliquots of culture supernatants were transferred to OptiPlate-384 (PerkinElmer) microplates. The level of IL-2 secretion in the supernatants was then determined using the IL-2 (human) AlphaLISA Detection Kit (PerkinElmer) according to the manufacturer's recommendations. The AlphaLISA signal was measured using an EnVision plate reader (PerkinElmer). EC₅₀ values were determined by fitting the data to a standard 4-parameter logistic equation. See Table 7 for data for select compounds (IL-2 Secretion (EC₅₀)).

TABLE 7
Potency of select compounds.

| Example # | Cbl-b Binding (IC₅₀) | IL-2 Secretion (EC₅₀) |
|---|---|---|
| 1 | +++ | +++ |
| 2 | +++ | +++ |
| 3 | +++ | +++ |
| 4 | +++ | +++ |
| 5 | +++ | +++ |
| 6 | +++ | + |
| 7 | +++ | +++ |
| 8 | +++ | ++ |
| 9 | +++ | +++ |
| 10 | +++ | +++ |
| 11 | +++ | ++ |
| 12 | +++ | ++ |
| 13 | +++ | ++ |
| 14 | +++ | +++ |
| 15 | +++ | +++ |
| 16 | +++ | ++ |
| 17 | +++ | +++ |
| 18 | +++ | +++ |
| 19 | +++ | ++ |
| 20 | +++ | +++ |
| 21 | +++ | +++ |
| 22 | +++ | ++ |
| 23 | +++ | +++ |
| 24 | +++ | +++ |
| 25 | +++ | ++ |
| 26 | +++ | ++ |
| 27 | ++ | − |
| 28 | ++ | − |
| 29 | ++ | + |
| 30 | ++ | − |
| 31 | ++ | − |
| 32 | +++ | + |
| 33 | +++ | ++ |
| 34 | +++ | + |
| 35 | +++ | ++ |
| 36 | ++ | + |
| 37 | ++ | − |
| 38 | ++ | + |
| 39 | +++ | +++ |
| 40 | +++ | ++ |
| 41 | +++ | ++ |
| 42 | +++ | +++ |
| 43 | +++ | +++ |
| 44 | ++ | + |
| 45 | +++ | +++ |
| 46 | ++ | + |
| 47 | ++ | + |
| 48 | +++ | +++ |
| 49 | +++ | +++ |
| 50 | +++ | +++ |
| 51 | ++ | + |
| 52 | + | − |
| 53 | +++ | ++ |
| 54 | +++ | ++ |
| 55 | +++ | +++ |
| 56 | +++ | +++ |
| 57 | +++ | +++ |
| 58 | +++ | +++ |
| 59 | +++ | +++ |
| 60 | +++ | ++ |
| 61 | +++ | +++ |
| 62 | +++ | +++ |
| 63 | +++ | ++ |
| 64 | +++ | ++ |
| 65 | +++ | ++ |
| 66 | ++ | + |
| 67 | +++ | +++ |
| 68 | +++ | +++ |
| 69 | +++ | ++ |
| 70 | +++ | ++ |
| 71 | +++ | +++ |
| 72 | +++ | ++ |
| 73 | ++ | + |
| 74 | ++ | + |
| 75 | ++ | + |
| 76 | +++ | +++ |
| 77 | +++ | n.d. |
| 78 | +++ | ++ |
| 79 | ++ | ++ |
| 80 | ++ | ++ |
| 81 | ++ | ++ |
| 82 | +++ | +++ |
| 83 | +++ | +++ |
| 84 | +++ | +++ |
| 85 | +++ | +++ |
| 86 | +++ | n.d. |
| 87 | +++ | − |
| 88 | +++ | +++ |
| 89 | +++ | +++ |
| 90 | +++ | +++ |
| 91 | +++ | +++ |
| 92 | +++ | +++ |
| 93 | +++ | +++ |
| 94 | +++ | n.d. |
| 95 | +++ | +++ |
| 96 | +++ | n.d. |
| 97 | +++ | n.d. |
| 98 | +++ | n.d. |
| 99 | +++ | n.d. |
| 100 | +++ | n.d. |
| 101 | +++ | n.d. |
| 102 | +++ | +++ |
| 103 | +++ | n.d. |
| 104 | +++ | n.d. |
| 105 | +++ | +++ |
| 106 | +++ | +++ |
| 107 | +++ | +++ |
| 108 | +++ | n.d. |
| 109 | +++ | n.d. |
| 110 | +++ | n.d. |
| 111 | +++ | n.d. |
| 112 | +++ | n.d. |
| 113 | +++ | n.d. |
| 114 | +++ | n.d. |
| 115 | +++ | n.d. |
| 116 | +++ | n.d. |
| 117 | +++ | n.d. |
| 118 | +++ | n.d. |
| 119 | +++ | n.d. |
| 120 | +++ | n.d. |
| 121 | +++ | n.d. |
| 122 | +++ | n.d. |
| 123 | +++ | n.d. |
| 124 | +++ | n.d. |
| 125 | +++ | n.d. |
| 126 | +++ | n.d. |
| 127 | +++ | n.d. |
| 128 | +++ | n.d. |
| 129 | +++ | n.d. |
| 130 | +++ | n.d. |
| 131 | +++ | n.d. |
| 132 | +++ | n.d. |
| 133 | +++ | n.d. |
| 134 | +++ | n.d. |
| 135 | +++ | n.d. |
| 136 | +++ | n.d. |
| 137 | +++ | n.d. |
| 138 | +++ | n.d. |
| 139 | +++ | n.d. |
| 140 | +++ | n.d. |
| 141 | +++ | n.d. |
| 142 | +++ | n.d. |
| 143 | +++ | n.d. |
| 144 | +++ | +++ |
| 145 | +++ | n.d. |
| 146 | +++ | n.d. |
| 147 | +++ | n.d. |
| 148 | +++ | n.d. |
| 149 | +++ | n.d. |
| 150 | +++ | n.d. |
| 151 | +++ | n.d. |
| 152 | +++ | n.d. |
| 153 | +++ | n.d. |
| 154 | +++ | +++ |
| 155 | +++ | n.d. |
| 156 | +++ | n.d. |
| 157 | +++ | n.d. |
| 158 | +++ | n.d. |
| 159 | +++ | n.d. |
| 160 | +++ | n.d. |
| 161 | +++ | n.d. |
| 162 | +++ | n.d. |
| 163 | +++ | n.d. |
| 164 | +++ | n.d. |
| 165 | +++ | n.d. |
| 166 | +++ | n.d. |
| 167 | +++ | n.d. |
| 168 | +++ | n.d. |
| 169 | +++ | n.d. |
| 170 | +++ | n.d. |
| 171 | +++ | n.d. |
| 172 | +++ | n.d. |
| 173 | +++ | n.d. |
| 174 | +++ | n.d. |
| 175 | +++ | n.d. |
| 176 | +++ | n.d. |
| 177 | +++ | n.d. |
| 178 | +++ | n.d. |
| 179 | +++ | n.d. |
| 180 | +++ | n.d. |
| 181 | +++ | n.d. |
| 182 | +++ | n.d. |
| 183 | +++ | n.d. |
| 184 | +++ | n.d. |
| 185 | +++ | n.d. |
| 186 | +++ | n.d. |
| 187 | +++ | n.d. |
| 188 | +++ | n.d. |
| 189 | +++ | n.d. |
| 190 | +++ | n.d. |
| 191 | +++ | n.d. |
| 192 | +++ | n.d. |
| 193 | +++ | n.d. |
| 194 | +++ | n.d. |
| 195 | +++ | n.d. |
| 196 | +++ | n.d. |
| 197 | +++ | n.d. |
| 198 | +++ | n.d. |
| 199 | +++ | n.d. |
| 200 | +++ | n.d. |
| 201 | +++ | n.d. |
| 202 | +++ | n.d. |
| 203 | +++ | n.d. |
| 204 | +++ | n.d. |
| 205 | +++ | n.d. |
| 206 | +++ | n.d. |
| 207 | +++ | n.d. |
| 208 | +++ | n.d. |
| 209 | +++ | n.d. |
| 210 | +++ | n.d. |
| 211 | +++ | n.d. |
| 212 | +++ | n.d. |
| 213 | +++ | n.d. |
| 214 | +++ | n.d. |
| 215 | +++ | n.d. |
| 216 | +++ | n.d. |
| 217 | +++ | n.d. |
| 218 | +++ | n.d. |
| 219 | +++ | n.d. |
| 220 | +++ | n.d. |
| 221 | +++ | n.d. |
| 222 | +++ | n.d. |
| 223 | +++ | n.d. |
| 224 | +++ | n.d. |
| 225 | +++ | n.d. |
| 226 | +++ | n.d. |
| 227 | +++ | n.d. |
| 228 | +++ | n.d. |
| 229 | +++ | n.d. |
| 230 | +++ | n.d. |
| 231 | +++ | n.d. |
| 232 | +++ | n.d. |
| 233 | +++ | n.d. |
| 234 | +++ | n.d. |
| 235 | +++ | n.d. |
| 236 | +++ | n.d. |
| 237 | +++ | n.d. |
| 238 | +++ | n.d. |
| 239 | +++ | n.d. |
| 240 | +++ | n.d. |
| 241 | +++ | n.d. |
| 242 | +++ | n.d. |
| 243 | +++ | n.d. |
| 244 | +++ | n.d. |
| 245 | +++ | n.d. |
| 246 | +++ | n.d. |
| 247 | +++ | n.d. |
| 248 | +++ | n.d. |
| 249 | +++ | n.d. |
| 250 | +++ | n.d. |
| 251 | +++ | n.d. |
| 252 | +++ | n.d. |
| 253 | +++ | n.d. |
| 254 | +++ | n.d. |
| 255 | +++ | n.d. |
| 256 | +++ | n.d. |
| 257 | +++ | n.d. |
| 258 | +++ | n.d. |
| 259 | +++ | n.d. |
| 260 | +++ | n.d. |
| 261 | +++ | n.d. |
| 262 | +++ | n.d. |
| 263 | +++ | n.d. |
| 264 | +++ | n.d. |
| 265 | +++ | n.d. |
| 266 | +++ | n.d. |
| 267 | +++ | n.d. |
| 268 | +++ | n.d. |
| 269 | +++ | n.d. |
| 270 | +++ | n.d. |
| 271 | +++ | n.d. |
| 272 | +++ | n.d. |
| 273 | +++ | n.d. |
| 274 | +++ | n.d. |
| 275 | +++ | n.d. |
| 276 | +++ | n.d. |
| 277 | +++ | n.d. |
| 278 | +++ | n.d. |
| 279 | +++ | n.d. |
| 280 | +++ | n.d. |
| 281 | +++ | n.d. |
| 282 | +++ | n.d. |
| 283 | +++ | n.d. |
| 284 | +++ | n.d. |
| 285 | +++ | n.d. |
| 286 | +++ | n.d. |
| 287 | +++ | n.d. |
| 288 | +++ | n.d. |
| 289 | +++ | n.d. |
| 290 | +++ | n.d. |
| 291 | +++ | n.d. |
| 292 | +++ | n.d. |
| 293 | +++ | n.d. |
| 294 | +++ | n.d. |
| 295 | +++ | n.d. |
| 296 | +++ | n.d. |
| 297 | +++ | n.d. |
| 298 | +++ | n.d. |
| 299 | +++ | n.d. |
| 300 | +++ | n.d. |
| 301 | +++ | n.d. |
| 302 | +++ | n.d. |
| 303 | +++ | n.d. |
| 304 | +++ | n.d. |
| 305 | +++ | n.d. |
| 306 | +++ | n.d. |
| 307 | +++ | n.d. |
| 308 | +++ | n.d. |
| 309 | +++ | n.d. |
| 310 | +++ | n.d. |
| 311 | +++ | n.d. |
| 312 | +++ | n.d. |
| 313 | +++ | n.d. |
| 314 | +++ | n.d. |
| 315 | +++ | n.d. |
| 316 | +++ | n.d. |
| 317 | +++ | n.d. |
| 318 | +++ | n.d. |
| 319 | +++ | n.d. |
| 320 | +++ | n.d. |
| 321 | +++ | n.d. |
| 322 | +++ | n.d. |
| 323 | +++ | n.d. |
| 324 | +++ | n.d. |
| 325 | +++ | n.d. |
| 326 | +++ | n.d. |
| 327 | +++ | n.d. |
| 328 | +++ | n.d. |
| 329 | +++ | n.d. |
| 330 | +++ | n.d. |
| 331 | +++ | n.d. |
| 332 | +++ | n.d. |
| 333 | +++ | n.d. |
| 334 | +++ | n.d. |
| 335 | +++ | n.d. |
| 336 | +++ | n.d. |
| 337 | +++ | n.d. |
| 338 | +++ | n.d. |
| 339 | +++ | n.d. |
| 340 | +++ | n.d. |
| 341 | +++ | n.d. |
| 342 | +++ | n.d. |
| 343 | +++ | n.d. |
| 344 | +++ | n.d. |
| 345 | +++ | n.d. |
| 346 | +++ | n.d. |
| 347 | +++ | n.d. |
| 348 | +++ | n.d. |
| 349 | +++ | n.d. |
| 350 | +++ | n.d. |
| 351 | +++ | n.d. |
| 352 | +++ | n.d. |
| 353 | +++ | n.d. |
| 354 | +++ | n.d. |
| 355 | +++ | n.d. |
| 356 | +++ | n.d. |
| 357 | +++ | n.d. |
| 358 | +++ | n.d. |
| 359 | +++ | n.d. |
| 360 | +++ | n.d. |
| 361 | +++ | n.d. |
| 362 | +++ | n.d. |
| 363 | +++ | n.d. |
| 364 | +++ | n.d. |
| 365 | +++ | n.d. |
| 366 | +++ | n.d. |
| 367 | +++ | n.d. |
| 368 | +++ | n.d. |
| 369 | +++ | n.d. |
| 370 | +++ | n.d. |
| 371 | +++ | n.d. |
| 372 | +++ | n.d. |
| 373 | +++ | n.d. |
| 374 | +++ | n.d. |
| 375 | +++ | n.d. |
| 376 | +++ | n.d. |
| 377 | +++ | n.d. |
| 378 | +++ | n.d. |
| 379 | +++ | n.d. |
| 380 | +++ | n.d. |
| 381 | +++ | n.d. |
| 382 | +++ | n.d. |
| 383 | +++ | n.d. |
| 384 | +++ | n.d. |
| 385 | +++ | n.d. |
| 386 | +++ | n.d. |
| 387 | +++ | n.d. |
| 388 | +++ | n.d. |
| 389 | +++ | n.d. |
| 390 | +++ | n.d. |
| 391 | +++ | n.d. |
| 392 | +++ | n.d. |
| 393 | +++ | n.d. |
| 394 | +++ | n.d. |
| 395 | +++ | n.d. |
| 396 | +++ | n.d. |
| 397 | +++ | n.d. |
| 398 | +++ | n.d. |
| 399 | +++ | n.d. |
| 400 | +++ | n.d. |
| 401 | +++ | n.d. |
| 402 | +++ | n.d. |
| 403 | +++ | n.d. |
| 404 | +++ | n.d. |
| 405 | +++ | n.d. |
| 406 | +++ | n.d. |
| 407 | +++ | n.d. |
| 408 | +++ | n.d. |
| 409 | +++ | n.d. |
| 410 | +++ | n.d. |
| 411 | +++ | n.d. |
| 412 | +++ | n.d. |

Less than 100 nM (+++), 100 nM to 1 μM (++), greater than 1 μM to 5 μM (+), >5 μM (−), n.d. = not determined

Particular embodiments of this disclosure are described herein, including the best mode known to the inventors for carrying out the disclosure. Upon reading the foregoing, description, variations of the disclosed embodiments may become apparent to individuals working in the art, and it is expected that those skilled artisans may employ such variations as appropriate. Accordingly, it is intended that the disclosure be practiced otherwise than as specifically described herein, and that the disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

All publications, patent applications, accession numbers, and other references cited in this specification are herein incorporated by reference for the purpose described herein.

1. A compound having a structure according to Formula I: 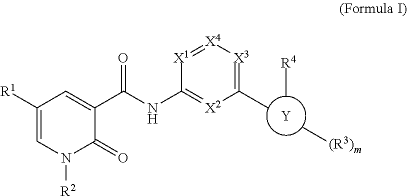
or a pharmaceutically acceptable salt thereof, wherein:
R¹ is selected from the group consisting of —H, —C₁-C₆ haloalkyl, —C₁-C₆ hydroxyalkyl, —C(O)NH₂, —C(O)—(C₁-C₆-alkyl), -(Q¹)-NR^{1a}R^{1b}, -(Q¹)-(C₃-C₇ cycloalkyl), -(Q¹)-phenyl, -(Q¹)-(5- to 6-membered heteroaryl), and -(Q¹)-(4- to 8-membered heterocycloalkyl); wherein said 5- to 6-membered heteroaryl has 1-3 ring heteroatoms independently selected from N, O, and S; said 4- to 8-membered heterocycloalkyl has 1-3 ring heteroatom or heteroatom groups independently selected from N, O, S, and S(O)₂; and said C₃-C₇ cycloalkyl, phenyl, 4- to 8-membered heterocycloalkyl, and 5- to 6-membered heteroaryl are unsubstituted or substituted with 1-2 substituents independently selected from halo, —OH, —C₁-C₃ alkyl, —C₁-C₃ alkoxy, and NH₂;
Q¹ is absent, unsubstituted —(C₁-C₃ alkylene)-, or —(C₁-C₃ alkylene)- substituted with 1-3 R^{q};
each R^{q} is independently halo, —OH, or —NH₂;
R^{1a} and R^{1b} are independently selected from the group consisting of —H, —C₁-C₆ alkyl, —C₁-C₆ haloalkyl, phenyl, —(C₁-C₆ alkylene)-O—(C₁-C₃ alkyl), —C₃-C₆ cycloalkyl, —(C₁-C₃ alkylene)-(C₃-C₆ cycloalkyl), —(C₁-C₃ alkylene)-O—(C₃-C₆ cycloalkyl), —S(O)₂(C₁-C₆ alkyl), 5- to 6-membered heteroaryl having 1-3 ring heteroatoms independently selected from N, O, and S, 4- to 8-membered heterocycloalkyl having 1-3 ring heteroatom or heteroatom groups independently selected from N, O, S, and S(O)₂, and —(C₁-C₃ alkylene)-(4- to 8-membered heterocycloalkyl) wherein said 4- to 8-membered heterocycloalkyl has 1-3 ring heteroatom or heteroatom groups independently selected from N, O, S, and S(O)₂; wherein said phenyl, —(C₁-C₆ alkylene)-O—(C₁-C₃ alkyl), —C₃-C₆ cycloalkyl, —(C₁-C₃ alkylene)-(C₃-C₆ cycloalkyl), 5- to 6-membered heteroaryl, 4- to 8-membered heterocycloalkyl, and —(C₁-C₃ alkylene)-(4- to 8-membered heterocycloalkyl) are unsubstituted or substituted with 1-3 R^{1c};
each R^{1c}, when present, is independently halo, —OH, —C₁-C₃ alkyl, —C₁-C₃ hydroxyalkyl, —C₁-C₃ haloalkyl, —C₁-C₃ alkoxy, or —(C₁-C₃ alkylene)-O—(C₁-C₃ alkyl);
R² is —H, —C₁-C₆ alkyl, —C₂-C₆ alkenyl, —C₁-C₆ haloalkyl, —C₁-C₆ hydroxyalkyl, —C₃-C₈ cycloalkyl, —(C₁-C₃ alkylene)-(C₃-C₈-cycloalkyl), and —(C₁-C₃ alkylene)-(3- to 8-membered heterocycloalkyl), wherein said 3- to 8-membered heterocycloalkyl has 1-3 ring heteroatoms independently selected from N, O, and S;
X¹, X² and X³ are each independently N or CH;
X⁴ is N or CR^{X};
R^{X}, when present, is —H, halo, —CN, —OH, —C₁-C₆ alkyl, —C₁-C₆ haloalkyl, —C₁-C₆ alkoxy, —C₁-C₆ haloalkoxy, —NR^{Xa}R^{Xb}, or —C₃-C₈ cycloalkyl;
R^{Xa} and R^{Xb} are independently —H, —C₁-C₃ alkyl, or —(C₁-C₃ alkylene)-NR^{Xc}R^{Xd};
R^{Xc} and R^{Xd} are independently —H, or —C₁-C₃ alkyl;
ring Y is —C₃-C₆ cycloalkylene, 3- to 6-membered heterocycloalkylene having 1-2 ring heteroatoms independently selected from N, O, and S, phenylene, or 5- to 6-membered heteroarylene having 1-2 ring heteroatoms independently selected from N, O, and S;
m is 0, 1, 2, or 3;
each R³ when present, is independently halo, —CN, —C₁-C₆ alkyl, —C₁-C₆ haloalkyl, —C(O)OR^{3a}, —C(O)NR^{3b}R^{3c}, —C₁-C₆ alkoxy, or —C₁-C₆ haloalkoxy;
R^{3a} is H or C₁-C₃ alkyl;
R^{3b} and R^{3c} are each independently —H or C₁-C₃ alkyl;
R⁴ is —C(O)NR^{4a}R^{4b}, or a 5- to 6-membered heteroaryl having 1 to 4 ring heteroatoms independently selected from N, O, and S, wherein said 5- to 6-membered heteroaryl is unsubstituted or substituted with 1-3 R^{4c};
R^{4a} and R^{4b} are each independently H, —C₁-C₆ alkyl, —C₁-C₆ haloalkyl, phenyl, or —(C₁-C₃ alkylene)-O—(C₁-C₃ alkyl); or
R^{4a} and R^{4b} taken together with the N atom to which they are attached form a 4- to 8-membered heterocycloalkyl having 0-1 additional ring heteroatom selected from N, O, and S; wherein said 4- to 8-membered heterocycloalkyl is unsubstituted or substituted with 1-3 substituents independently selected from the group consisting of halo, —CN, —C₁-C₆ alkyl, and —C₁-C₆ alkoxy; and
each R^{4c} is independently selected from the group consisting of —CN, —C₁-C₃ alkyl, —C₁-C₃ haloalkyl, and —C₁-C₃ hydroxyalkyl.

2. (canceled)

3. The compound of claim 1, or a pharmaceutically acceptable salt thereof, wherein X² is CH, X³ is CH, and/or X⁴ is CR^{X}.

4-5. (canceled)

6. The compound of claim 1, or a pharmaceutically acceptable salt thereof, wherein the compound has a structure according to Formula Ia:

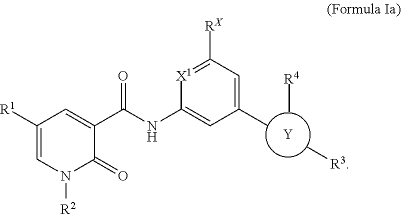

7. The compound of claim 6, or a pharmaceutically acceptable salt thereof, wherein:
R¹ is selected from the group consisting of —C₁-C₆ hydroxyalkyl, -(Q¹)-NR^{1a}R^{1b}, -(Q¹)-(C₃-C₇ cycloalkyl), -(Q¹)-phenyl, -(Q¹)-(5- to 6-membered heteroaryl), and -(Q¹)-(4- to 8-membered heterocycloalkyl); wherein said 5- to 6-membered heteroaryl and 4- to 8-membered heterocycloalkyl have 1-3 ring heteroatoms independently selected from N, O, and S; and wherein said phenyl, 4- to 8-membered heterocycloalkyl, and 5- to 6-membered heteroaryl are unsubstituted or substituted with 1-2 substituents independently selected from the group consisting of —C₁-C₃ alkyl, —C₁-C₃ alkoxy, and —NH₂;
Q¹ is absent, or unsubstituted —(C₁-C₃ alkylene)-;
R^{1a} and R^{1b} are independently selected from the group consisting of —H, —C₁-C₆ alkyl, —C₁-C₆ haloalkyl, —(C₁-C₃ alkylene)-O—(C₁-C₃ alkyl), —C₃-C₆ cycloalkyl, —(C₁-C₃ alkylene)-(C₃-C₆ cycloalkyl), and 4- to 8-membered heterocycloalkyl having 1-3 ring heteroatoms independently selected from N, O, and S, wherein said —(C₁-C₃ alkylene)-O—(C₁-C₃ alkyl), —C₃-C₆ cycloalkyl, —(C₁-C₃ alkylene)-(C₃-C₆ cycloalkyl), and 4- to 8-membered heterocycloalkyl are unsubstituted or substituted with 1-3 R^{1c};
each R^{1c}, when present, is independently halo, —C₁-C₃ alkyl, or —(C₁-C₃ alkylene)-O—(C₁-C₃ alkyl);
R² is —H, —C₁-C₆ alkyl, —C₂-C₆ alkenyl, —C₁-C₆ haloalkyl, —C₁-C₆ hydroxyalkyl, —C₃-C₈ cycloalkyl, —(C₁-C₃ alkylene)-(C₃-C₈-cycloalkyl), or —(C₁-C₃ alkylene)-(3- to 8-membered heterocycloalkyl), wherein said 3- to 8-membered heterocycloalkyl has 1-3 ring heteroatoms independently selected from N, O, and S;
X¹ is N or CH;
R^{X} is —H, halo, —CN, —C₁-C₆ alkoxy, —NH₂, or —C₃-C₈ cycloalkyl;
ring Y is —C₃-C₆ cycloalkylene, or phenylene;
R³ is halo, —CN, —C₁-C₆ alkyl, —C(O)OR^{3a}, —C(O)NH₂, or —C₁-C₆ alkoxy;
R^{3a} is C₁-C₃ alkyl;
R⁴ is —C(O)NR^{4a}R^{4b}, or a 5- to 6-membered heteroaryl having 1 to 3 heteroatoms independently selected from N, O, and S, wherein said 5- to 6-membered heteroaryl is unsubstituted or substituted with 1-3 R^{4c};
R^{4a} and R^{4b} taken together with the N atom to which they are attached form a 4- to 8-membered heterocycloalkyl having 0-1 additional ring heteroatom selected from N, O, and S; wherein said 4- to 8-membered heterocycloalkyl is unsubstituted or substituted with 1-3 halo; and
each R^{4c} is independently selected from the group consisting of —CN, —C₁-C₃ alkyl, and —C₁-C₃ haloalkyl.

8. The compound of claim 1, or a pharmaceutically acceptable salt thereof, wherein the compound has a structure according to Formula Ib or Formula Ic:

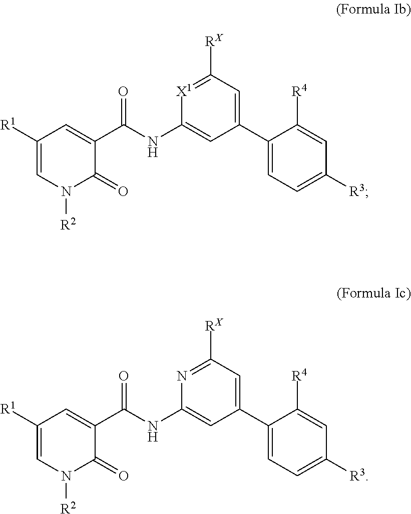

9. (canceled)

10. The compound according to claim 8, or a pharmaceutically acceptable salt thereof, wherein R⁴ is a 5- to 6-membered heteroaryl having 1-3 ring heteroatoms independently selected from N, and O, wherein said heteroaryl is substituted with 1-3 R^{4c}.

11. The compound according to claim 10, or a pharmaceutically acceptable salt thereof, where R⁴ is triazolyl, oxazolyl, imidazolyl, pyrazolyl, pyridinyl, or pyridazinyl, each of which is substituted with 1-3 R^{4c}.

12-14. (canceled)

15. The compound according to claim 1, or a pharmaceutically acceptable salt thereof, wherein each R^{4c} is independently —CH₃, —CH₂F, —CF₂H, —CF₃, or —CN.

16. (canceled)

17. The compound according to claim 1, or pharmaceutically acceptable salt thereof, wherein the compound has structure according to Formula Ig or Formula Ie:

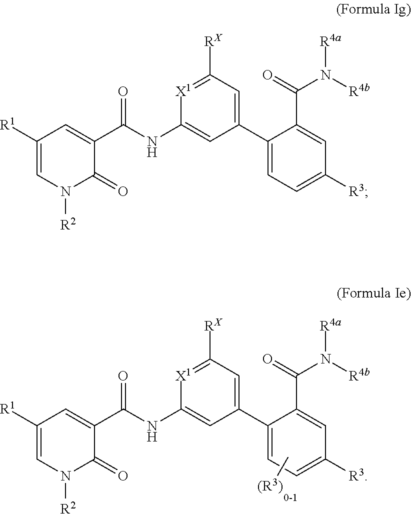

18. The compound according to claim 17, or a pharmaceutically acceptable salt thereof, wherein R^{4a} and R^{4b} are taken together with the N atom to which they are attached to form a 4- to 8-membered heterocycloalkyl having 0-1 additional ring heteroatom selected from N, O, and S; wherein said 4- to 8-membered heterocycloalkyl is unsubstituted or substituted with 1-3 halo.

19. The compound according to claim 18, or a pharmaceutically acceptable salt thereof, wherein R^{4a} and R^{4b} are taken together with the N atom to which they are attached to form an azetidine ring, wherein said azetidine ring is substituted with 1-2 halo.

20. The compound according to claim 1, or a pharmaceutically acceptable salt thereof, wherein each R³ is independently selected from the groups consisting of:
a) halo, —CN, —C(O)OR^{3a}, —C(O)NH₂, and —C₁-C₆ haloalkoxy; and
b) halo, —C₁-C₃ alkyl, and —C₁-C₃ alkoxy.

21. (canceled)

22. The compound according to claim 1, or a pharmaceutically acceptable salt thereof, wherein R³ is halo, —CN, or —OCF₂H.

23. (canceled)

24. The compound according to claim 1, or a pharmaceutically acceptable salt thereof, wherein R^{X} is halo, —CN, —C₁-C₆ alkoxy, —C₁-C₆ haloalkoxy, —NR^{Xa}R^{Xb}, or —C₃-C₈ cycloalkyl.

25-27. (canceled)

28. The compound according to claim 1, or a pharmaceutically acceptable salt thereof, wherein R² is —C₁-C₆ alkyl, —C₁-C₆ haloalkyl, —C₁-C₆ hydroxyalkyl, —C₃-C₈ cycloalkyl, —(C₁-C₃ alkylene)-(C₃-C₈-cycloalkyl), or —(C₁-C₃ alkylene)-(3- to 8-membered heterocycloalkyl), wherein said 3- to 8-membered heterocycloalkyl has 1-3 ring heteroatoms independently selected from N, O, and S.

29. The compound according to claim 1, or a pharmaceutically acceptable salt thereof, wherein R² is selected from the group consisting of:

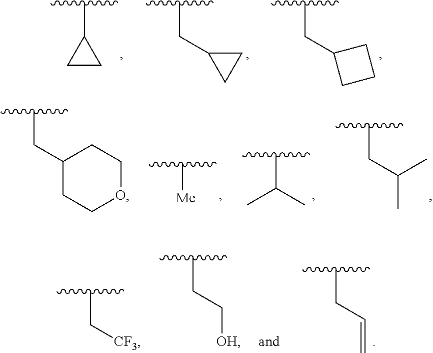

30-32. (canceled)

33. The compound according to claim 1, or a pharmaceutically acceptable salt thereof, wherein:
R¹ is —(CH₂)—NR^{1a}R^{1b}, or —(CH₂)-(4- to 8-membered heterocycloalkyl), wherein said 4- to 8-membered heterocycloalkyl has 1-2 ring N atoms, and is unsubstituted or substituted with 1-2 substituents independently selected from —C₁-C₃ alkyl and —C₁-C₃ alkoxy;
R^{1a} and R^{1b} are independently selected from the group consisting of —H, —C₁-C₆ alkyl, —C₁-C₆ haloalkyl, —(C₁-C₃ alkylene)-O—(C₁-C₃ alkyl), —C₃-C₆ cycloalkyl, —(C₁-C₃ alkylene)-(C₃-C₆ cycloalkyl), 4- to 8-membered heterocycloalkyl having 1-3 ring heteroatoms independently selected from N, O, and S, and —(C₁-C₃ alkylene)-(4- to 8-membered heterocycloalkyl) wherein said 4- to 8-membered heterocycloalkyl has 1-2 ring heteroatoms independently selected from N and O; wherein said —(C₁-C₃ alkylene)-O—(C₁-C₃ alkyl), —C₃-C₆ cycloalkyl, —(C₁-C₃ alkylene)-(C₃-C₆ cycloalkyl), 4- to 8-membered heterocycloalkyl, and —(C₁-C₃ alkylene)-(4- to 8-membered heterocycloalkyl) are unsubstituted or substituted with 1-3 R^{1c}; and
each R^{1c}, when present, is independently halo, —C₁-C₃ alkyl, or —(C₁-C₃ alkylene)-O—(C₁-C₃ alkyl).

34. (canceled)

35. The compound according to claim 33, or a pharmaceutically acceptable salt thereof, wherein R¹ is —(CH₂)—NR^{1a}R^{1b}; and one of R^{1a} and R^{1b} is —H.

36. The compound according to claim 33, or a pharmaceutically acceptable salt thereof, wherein R¹ is —(CH₂)-(4- to 6-membered heterocycloalkyl); wherein said 4- to 6-membered heterocycloalkyl has 1-2 ring N atoms and is unsubstituted or substituted with a —C₁-C₃ alkyl.

37. The compound according to claim 33, or a pharmaceutically acceptable salt thereof, wherein R¹ is selected from the group consisting of:

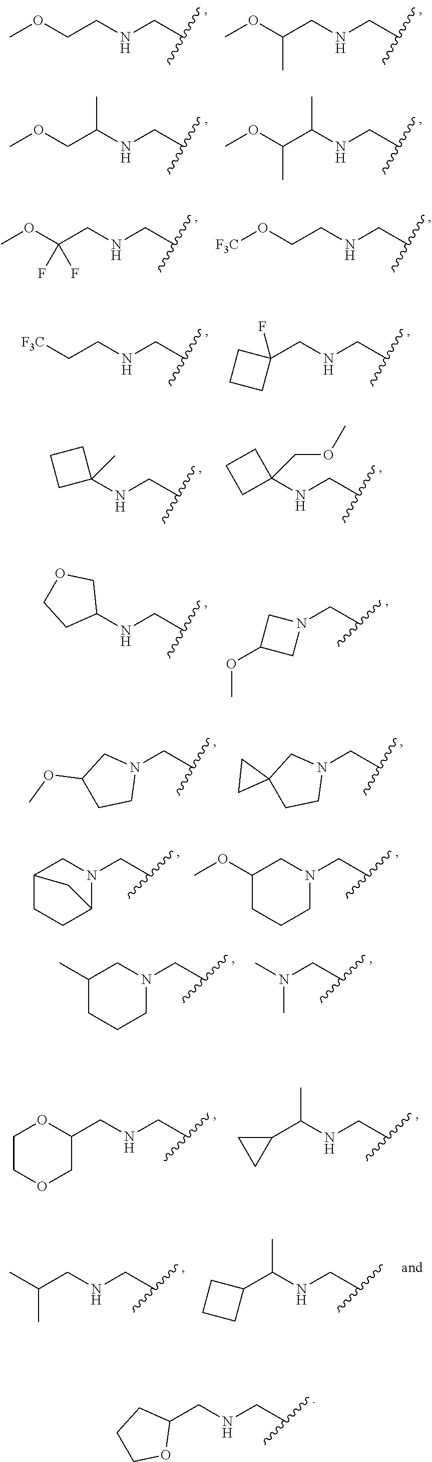

38. (canceled)

39. A compound, or a pharmaceutically acceptable salt thereof, selected from the group consisting of:

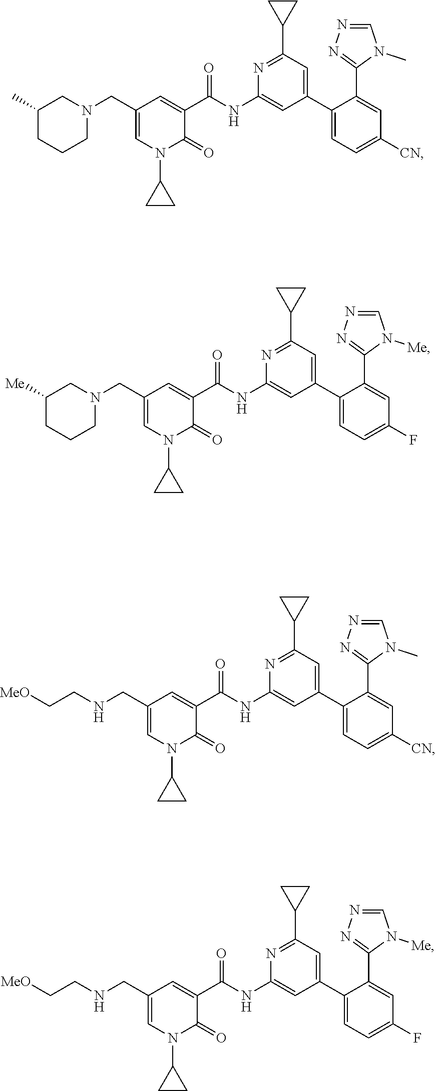

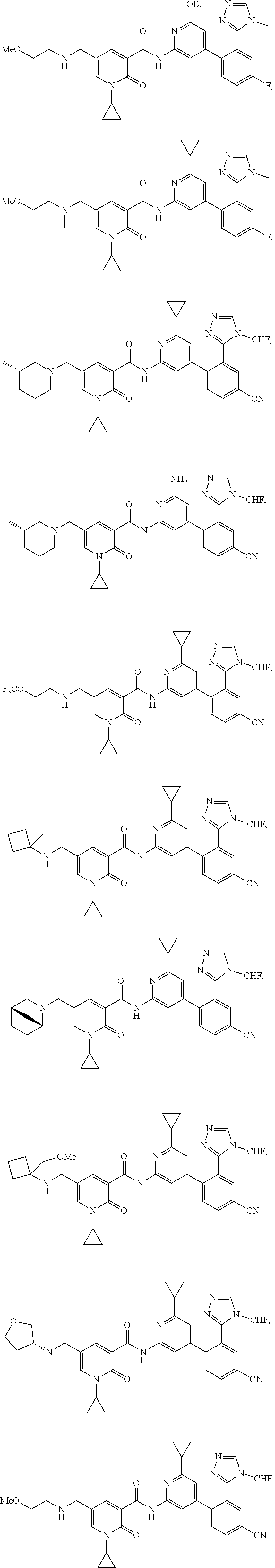

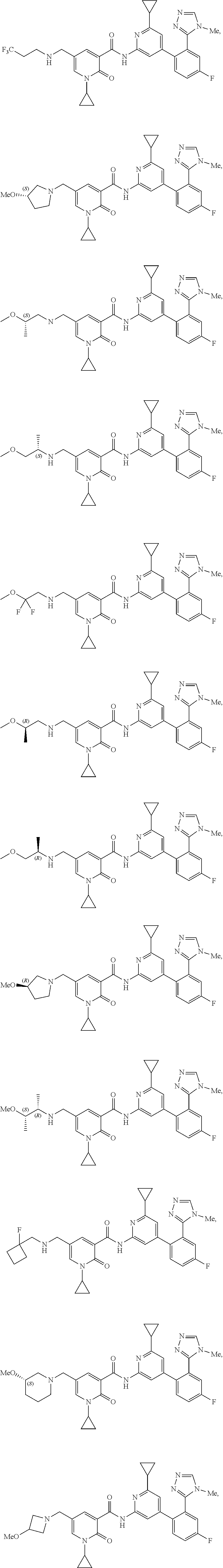

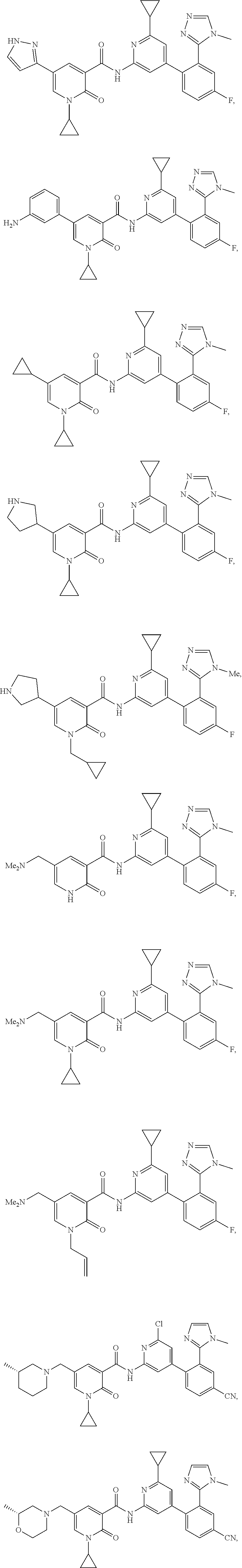

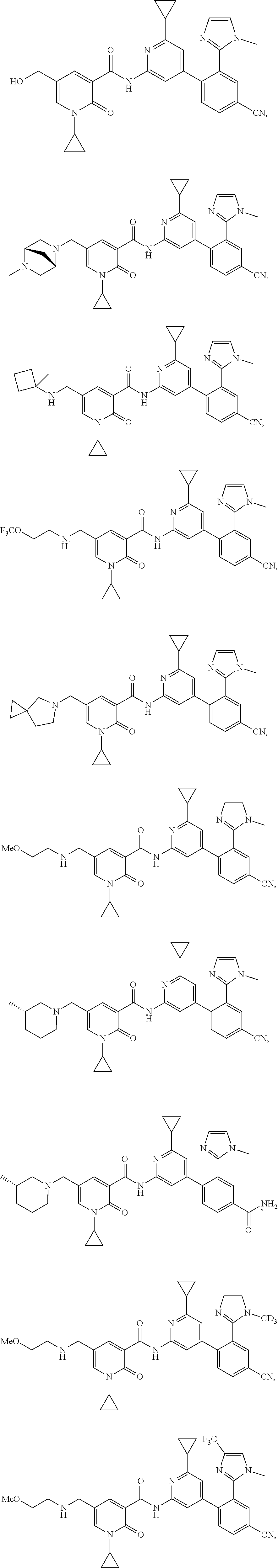

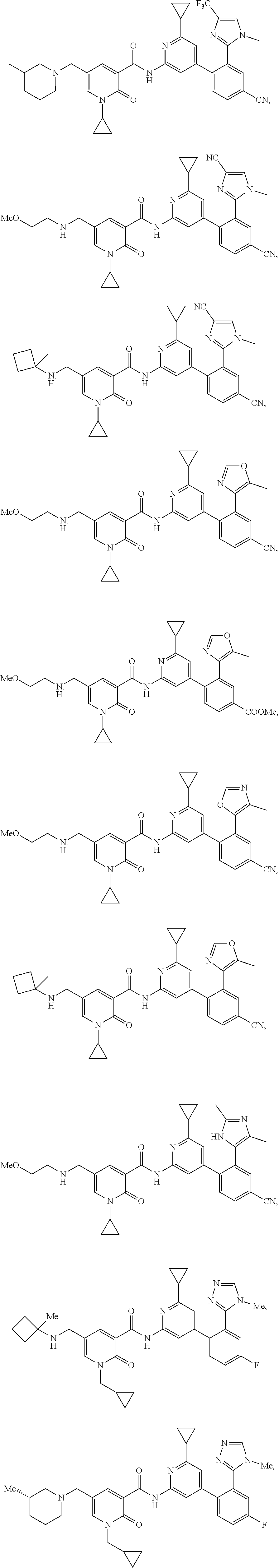

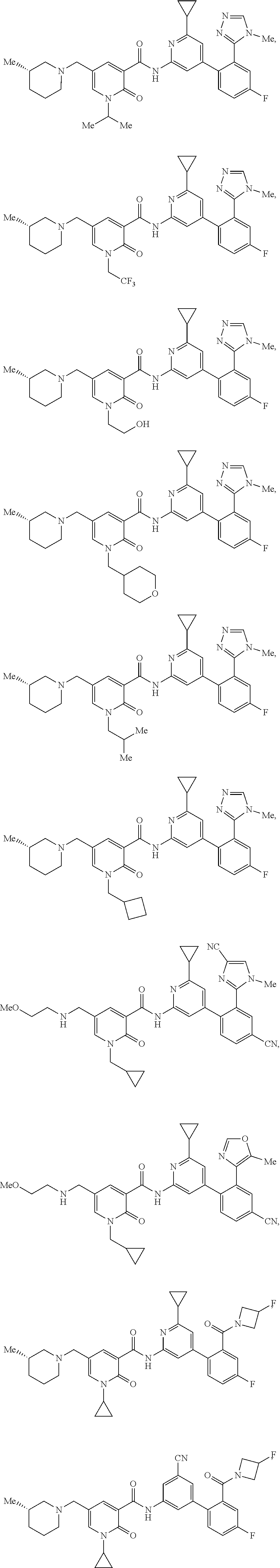

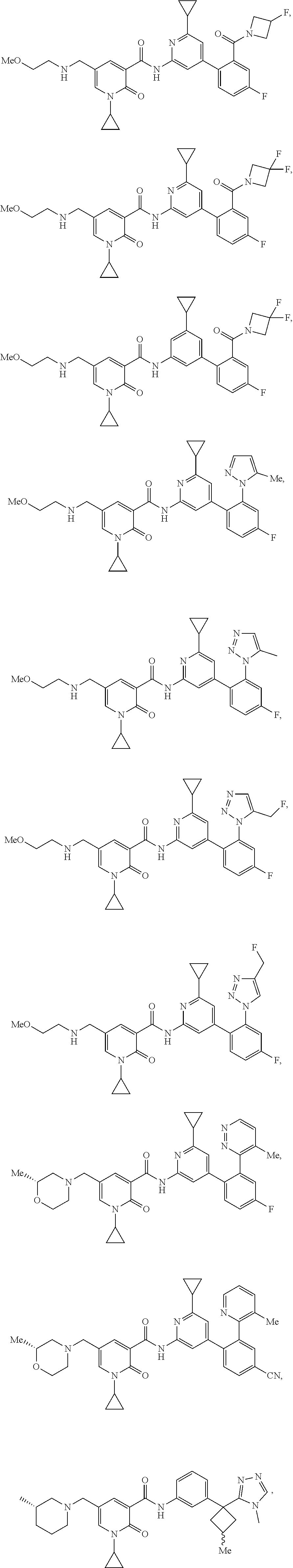

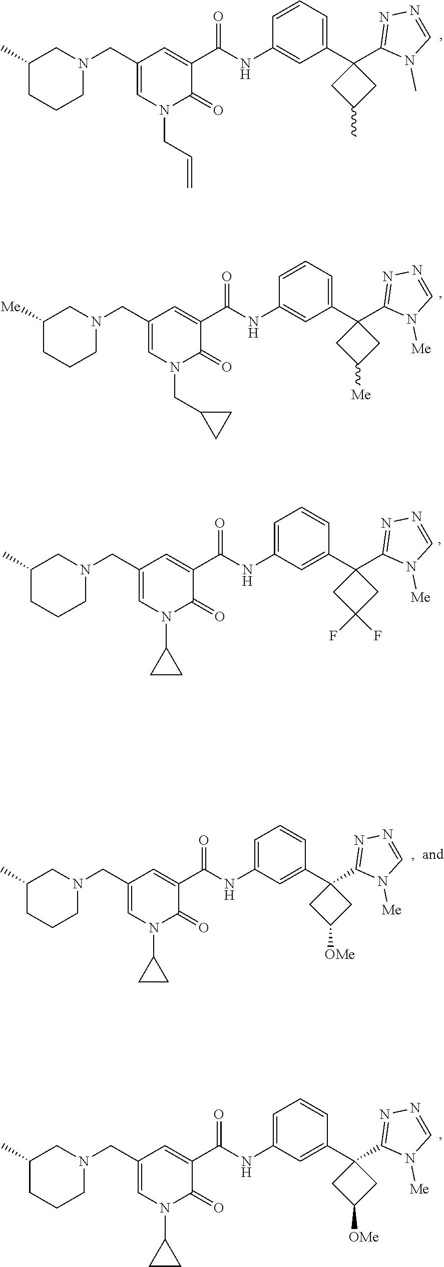

40. A compound selected from the group consisting of:

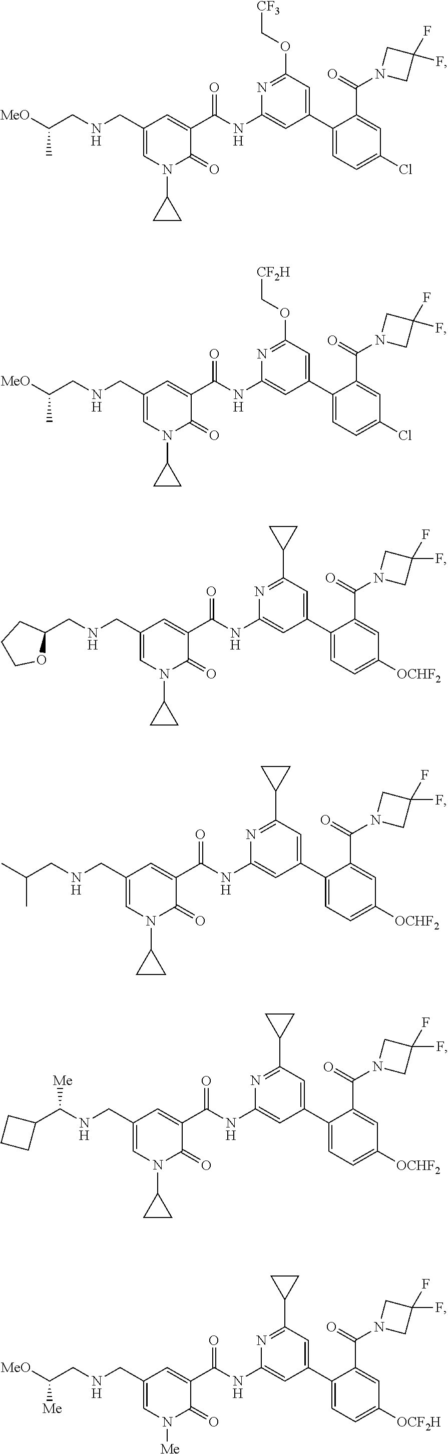

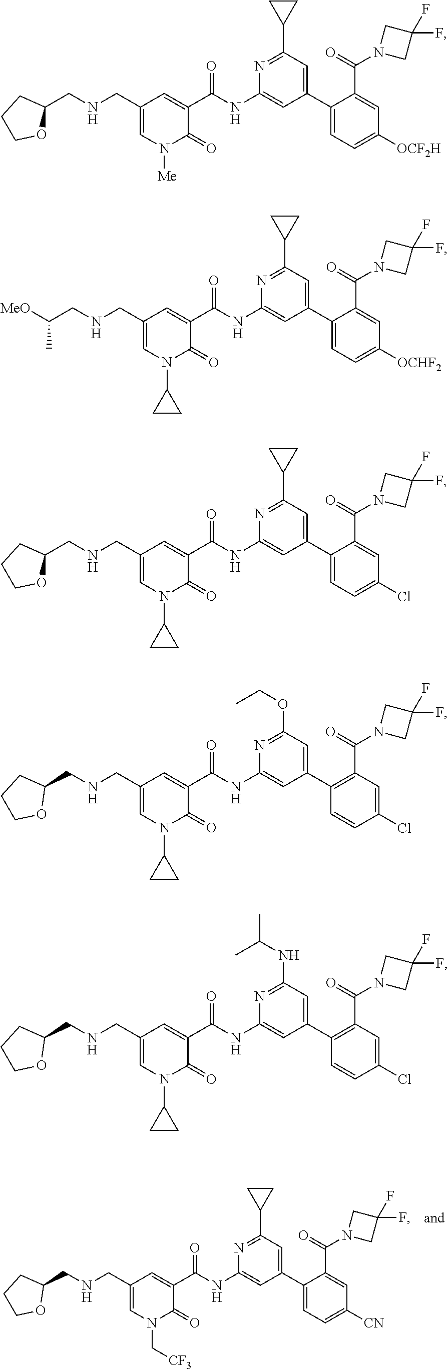

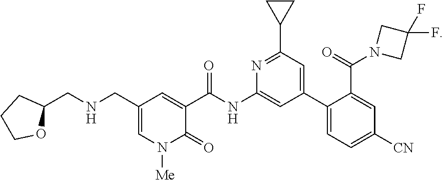 [SMILES: Cn1cc(CNC[C@@H]2CCCO2)cc(C(=O)Nc2cc(-c3ccc(C#N)cc3C(=O)N3CC(F)(F)C3)cc(C3CC3)n2)c1=O]

41. A compound selected from the group consisting of:

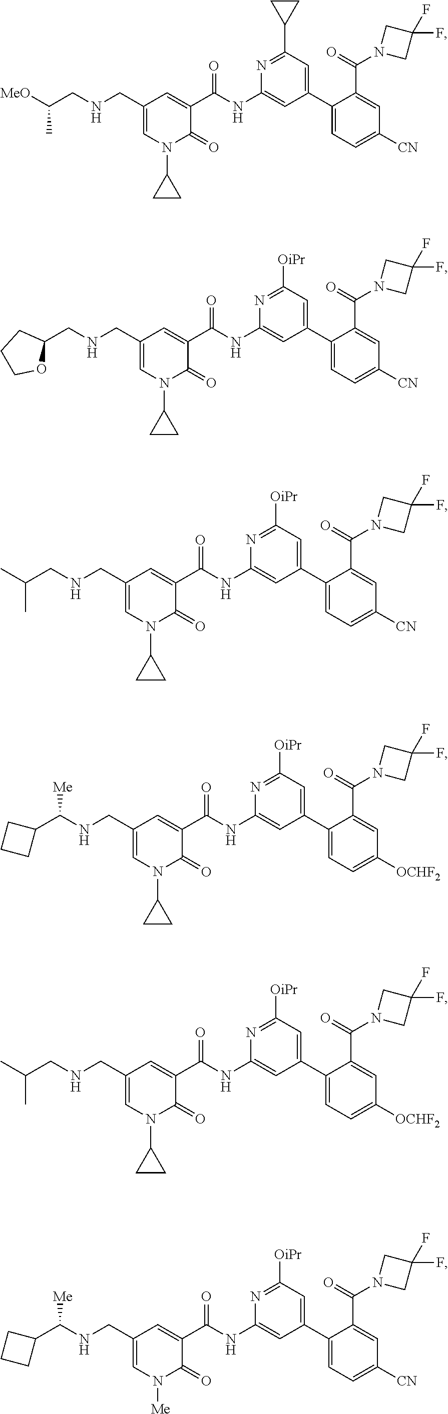

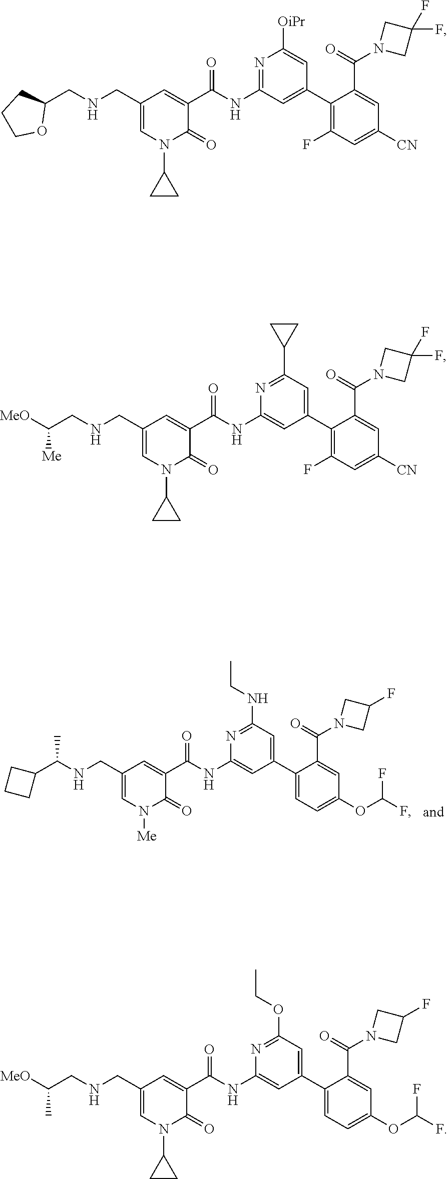 [SMILES: CC(C)Oc1cc(-c2c(F)cc(C#N)cc2C(=O)N2CC(F)(F)C2)cc(NC(=O)c2cc(CNC[C@@H]3CCCO3)cn(C3CC3)c2=O)n1 ; CO[C@@H](C)CNCc1cc(C(=O)Nc2cc(-c3c(F)cc(C#N)cc3C(=O)N3CC(F)(F)C3)cc(C3CC3)n2)c(=O)n(C2CC2)c1 ; CCNc1cc(-c2ccc(OC(F)F)cc2C(=O)N2CC(F)C2)cc(NC(=O)c2cc(CN[C@@H](C)C3CCC3)cn(C)c2=O)n1 ; CCOc1cc(-c2ccc(OC(F)F)cc2C(=O)N2CC(F)C2)cc(NC(=O)c2cc(CNC[C@H](C)OC)cn(C3CC3)c2=O)n1]

42. A compound selected from the group consisting of:

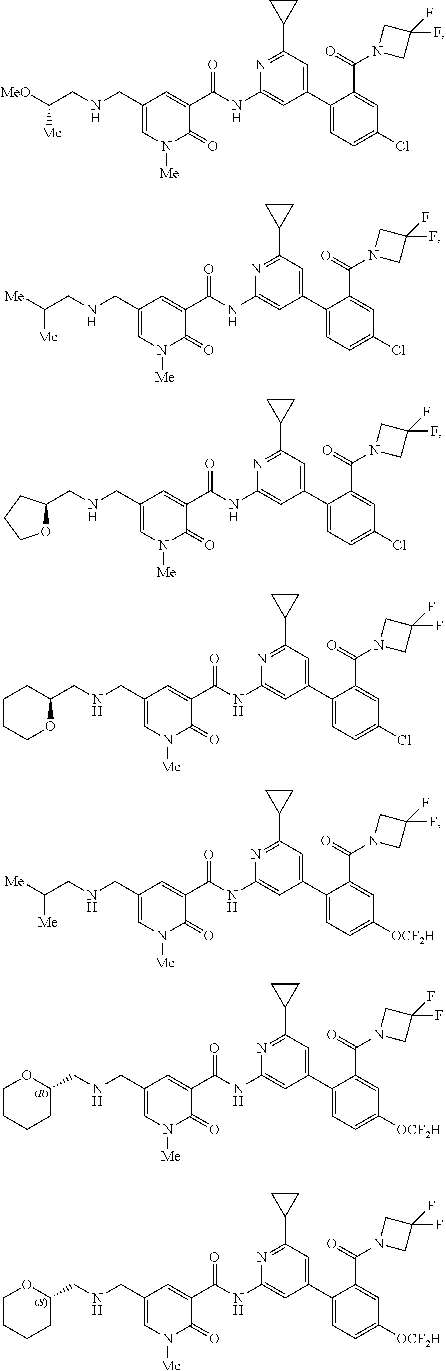

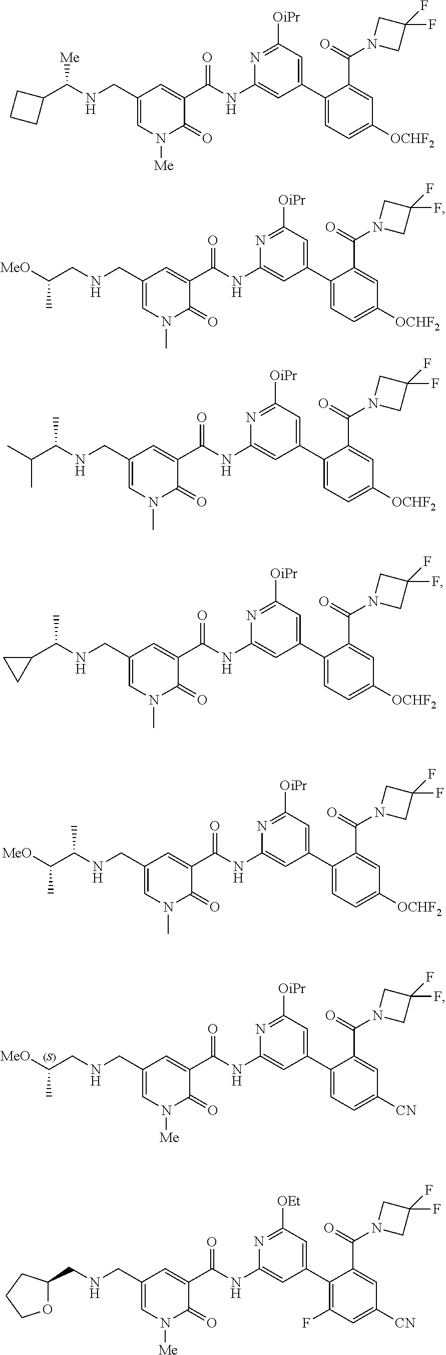

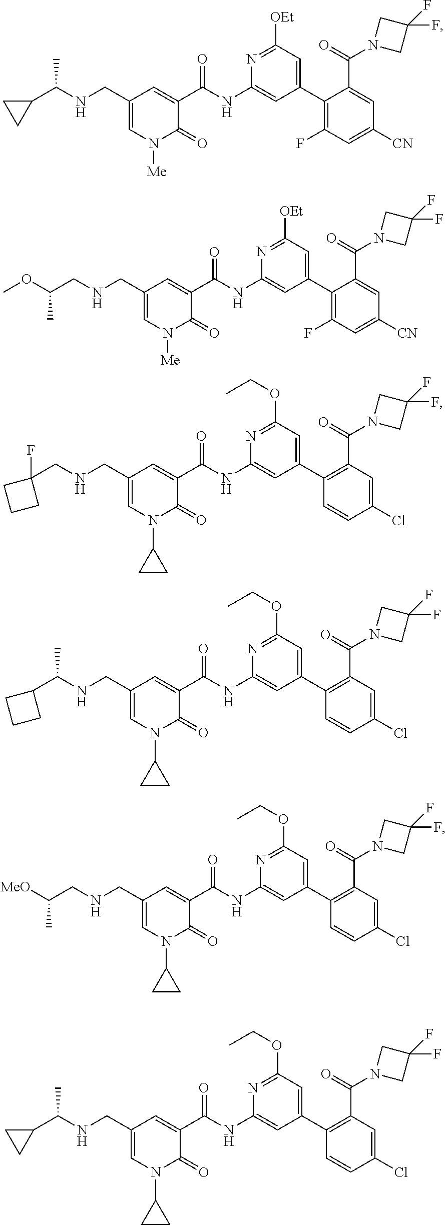

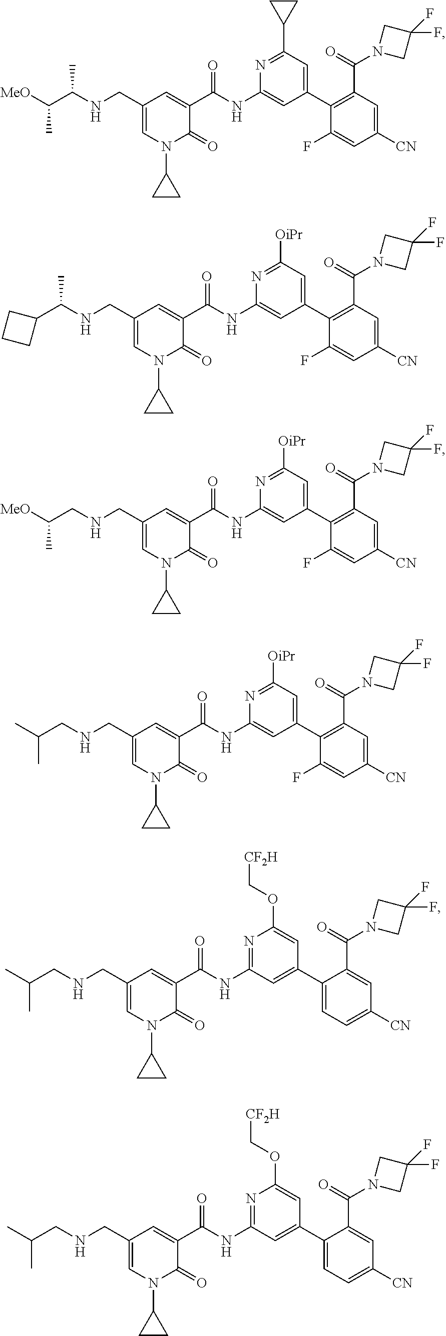

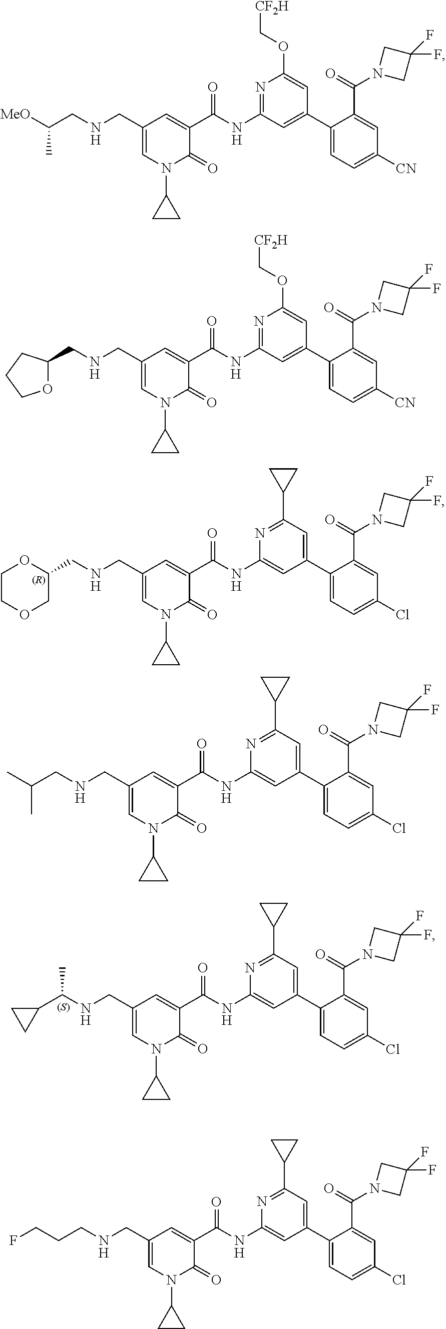

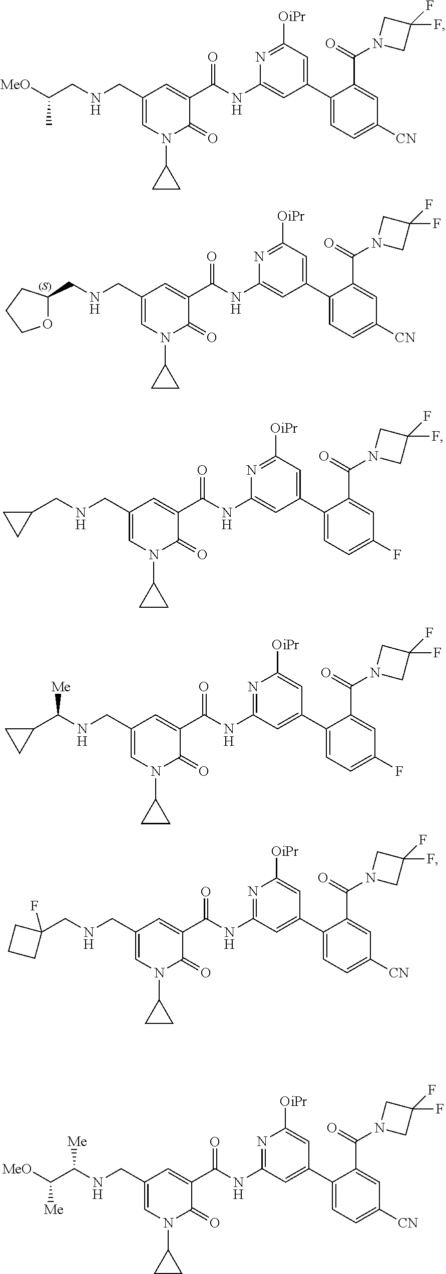

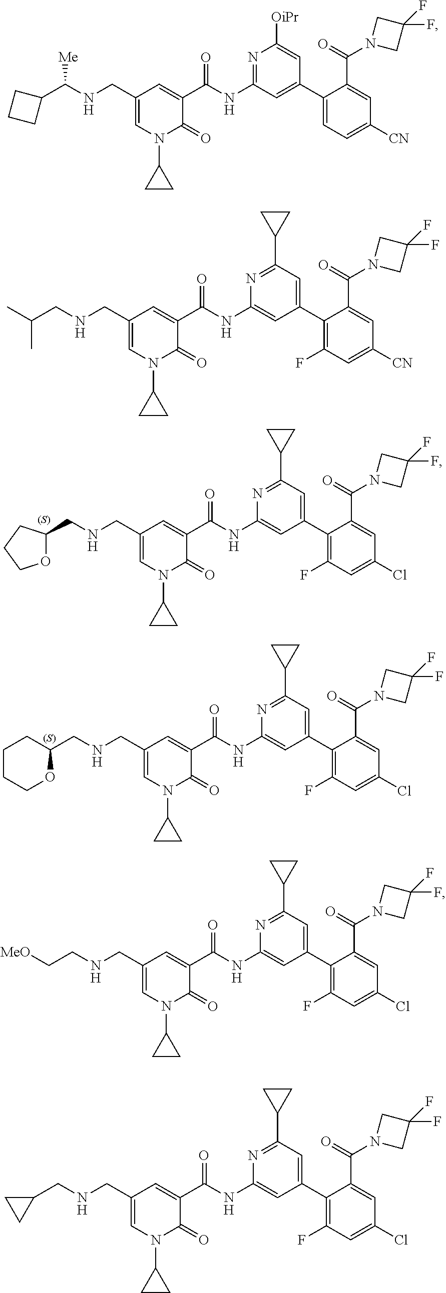

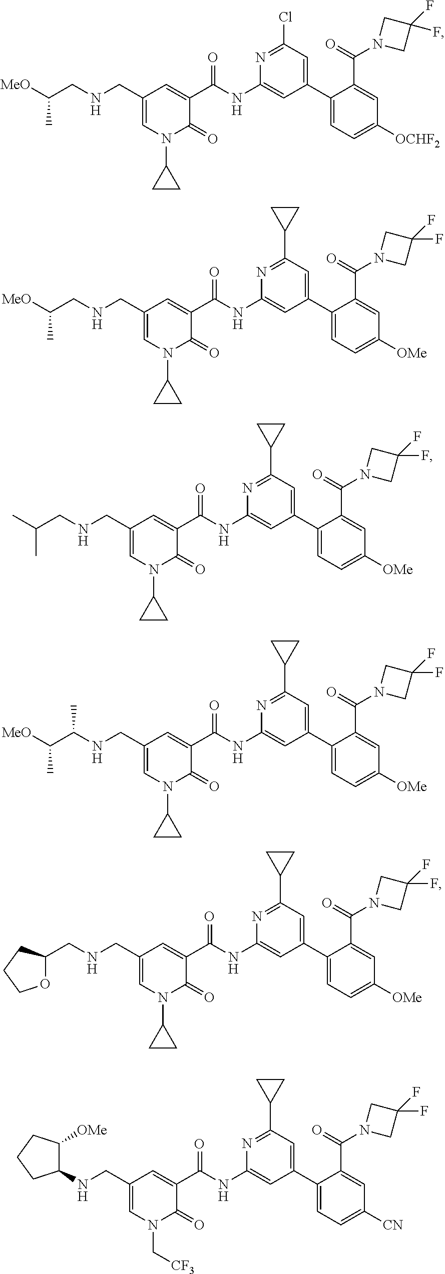

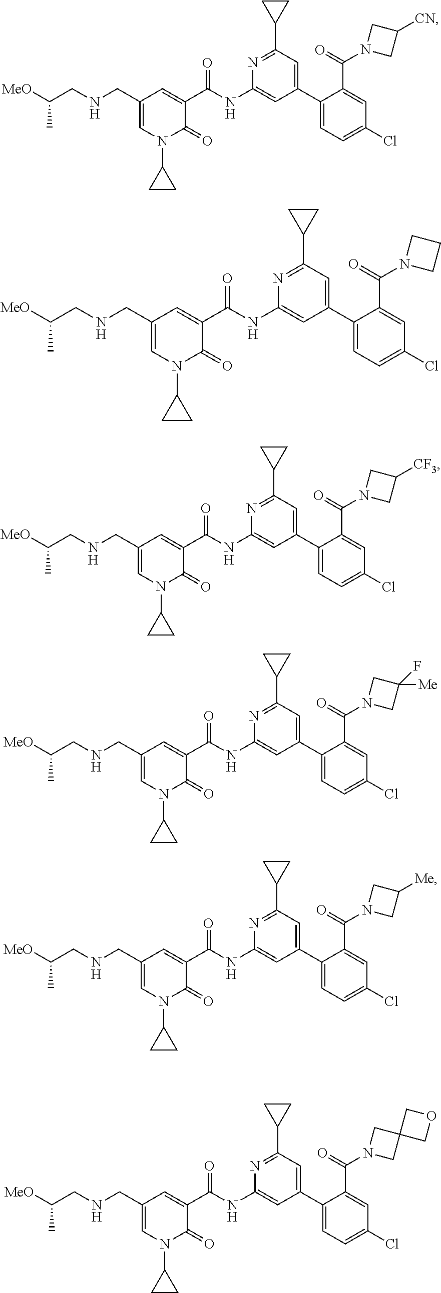

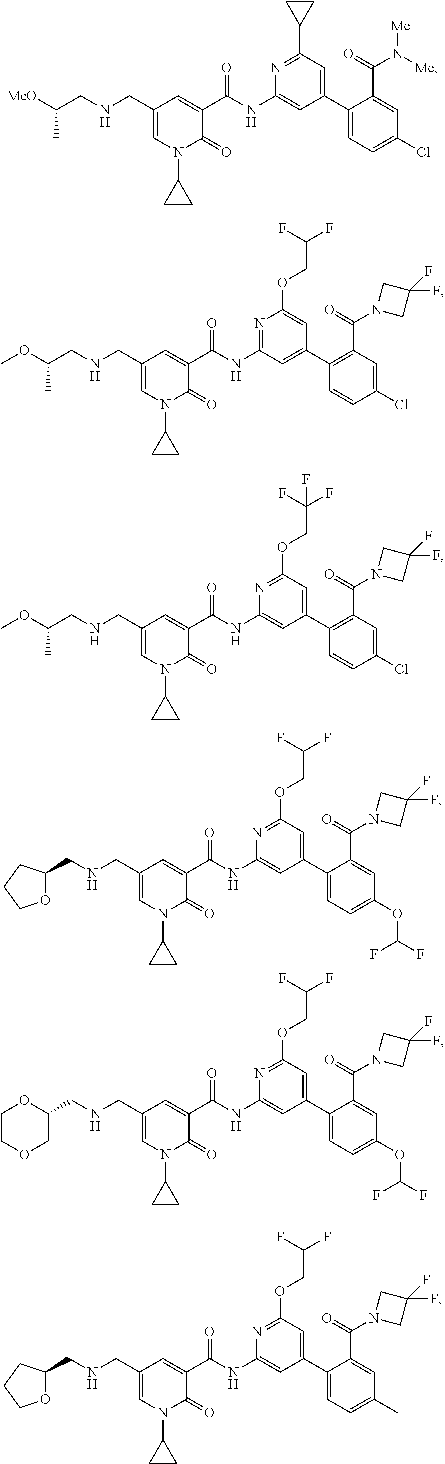

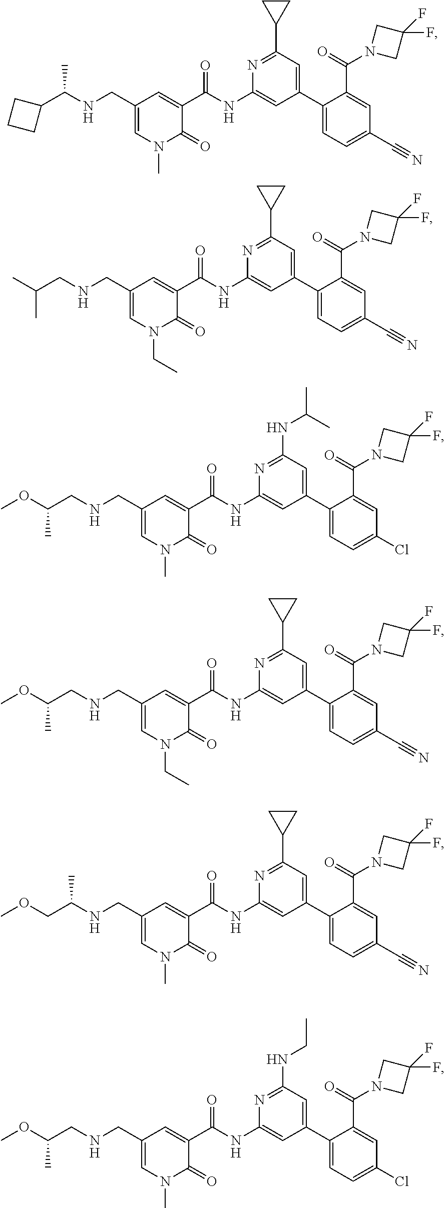

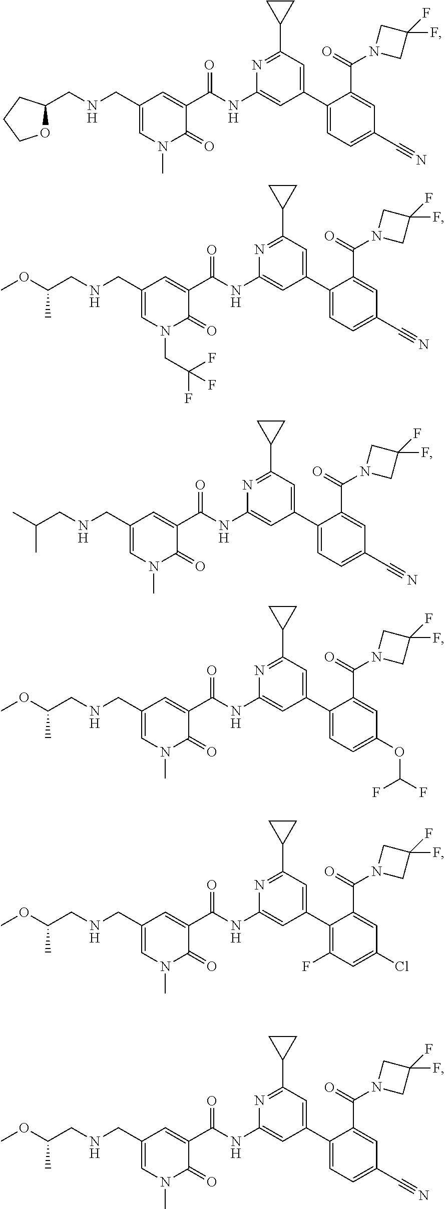

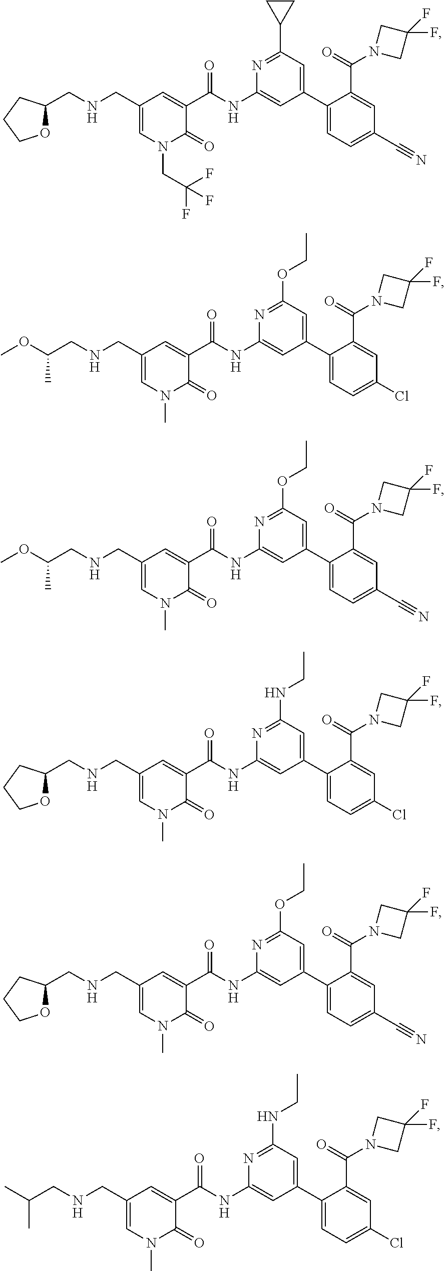

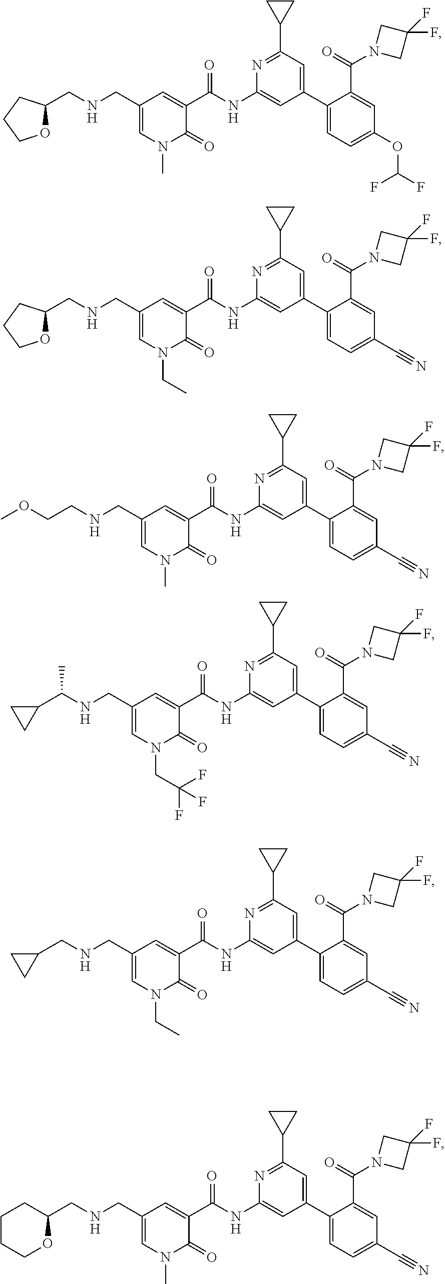

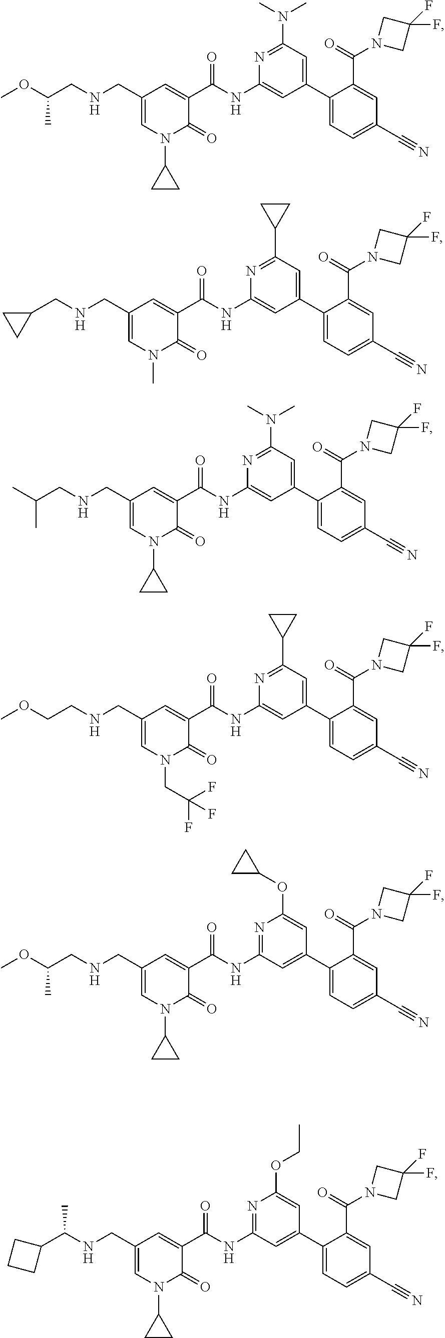

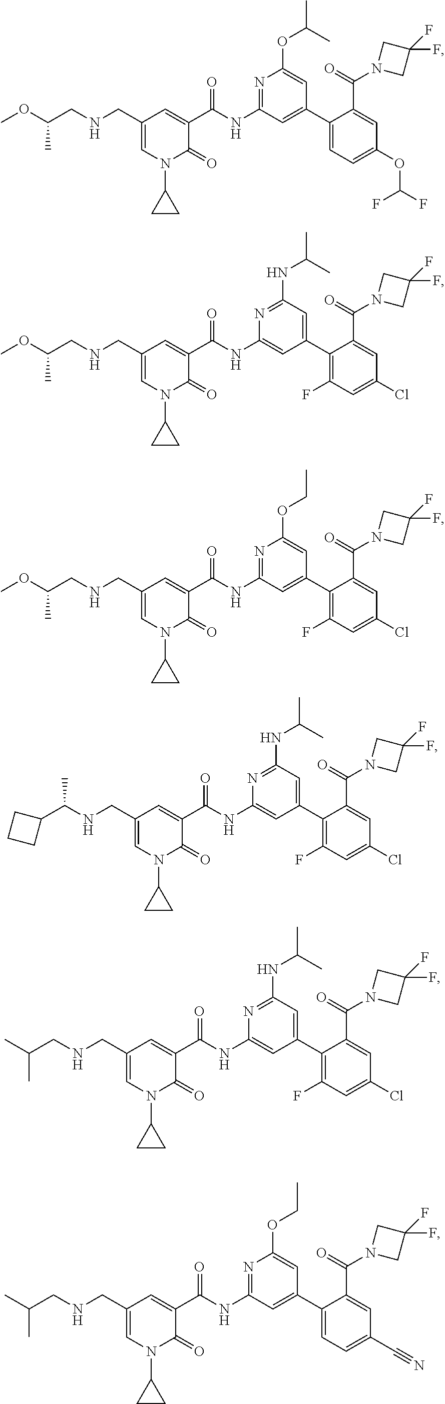

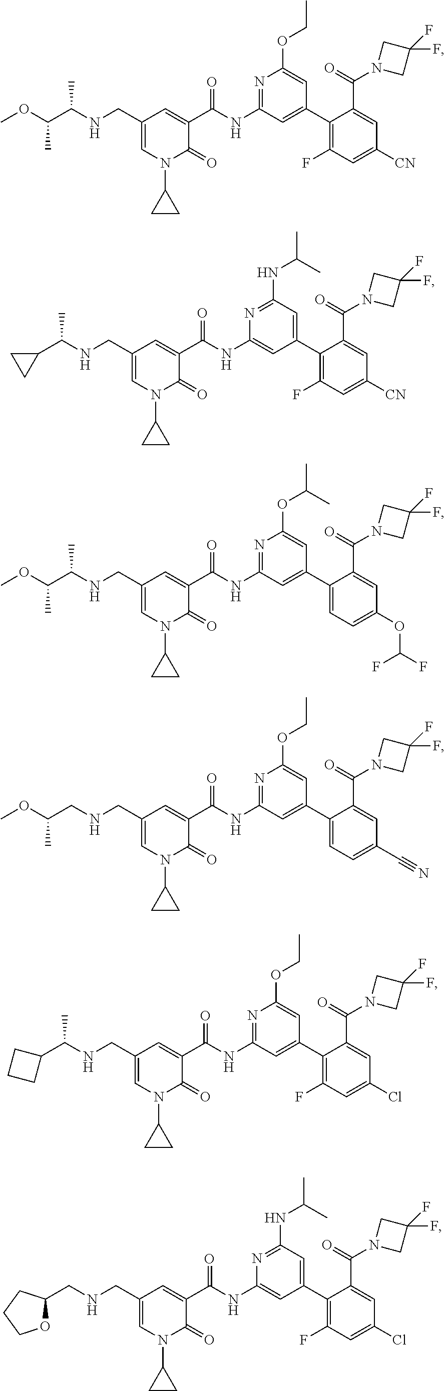

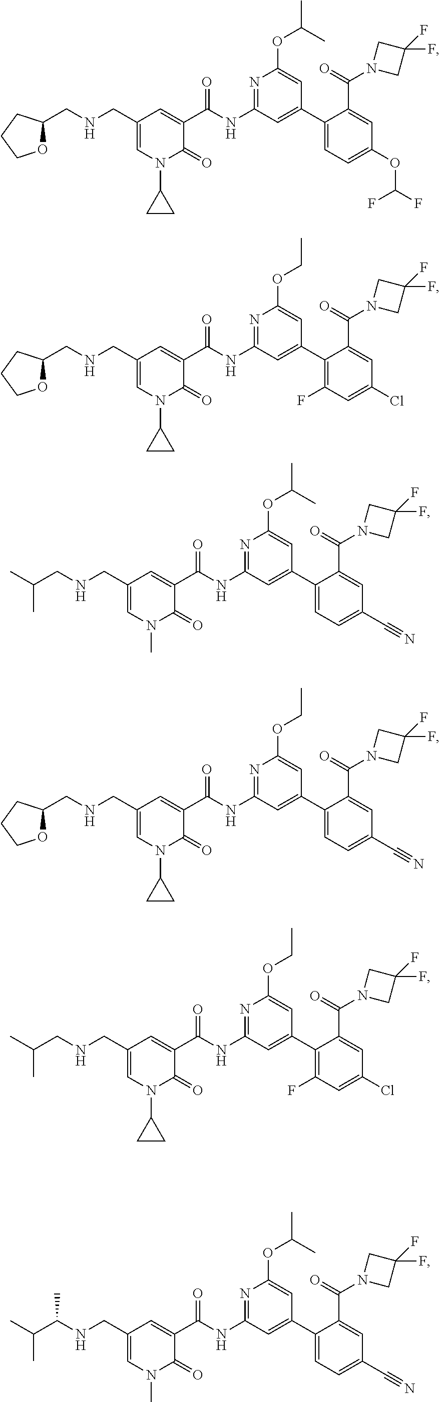

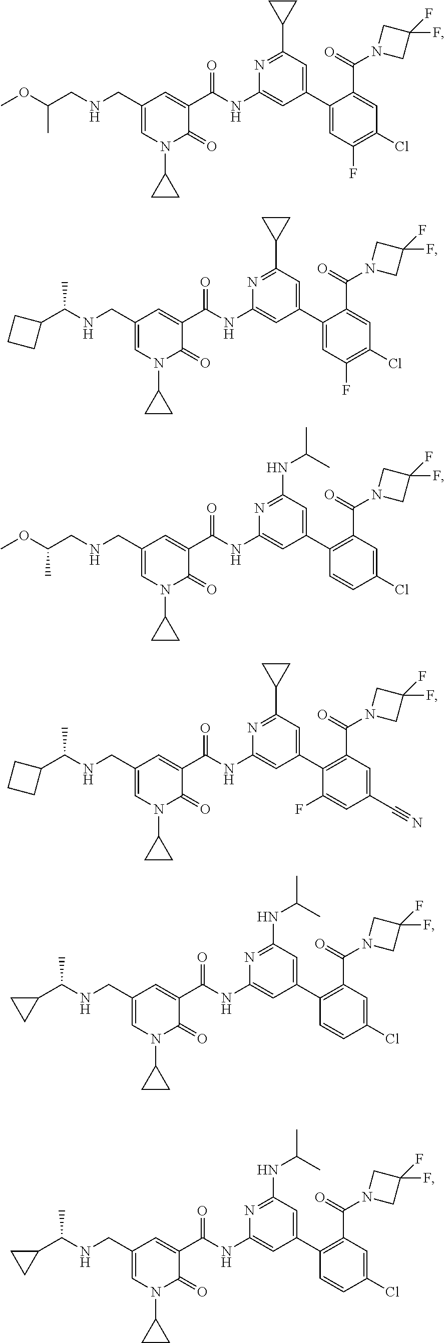

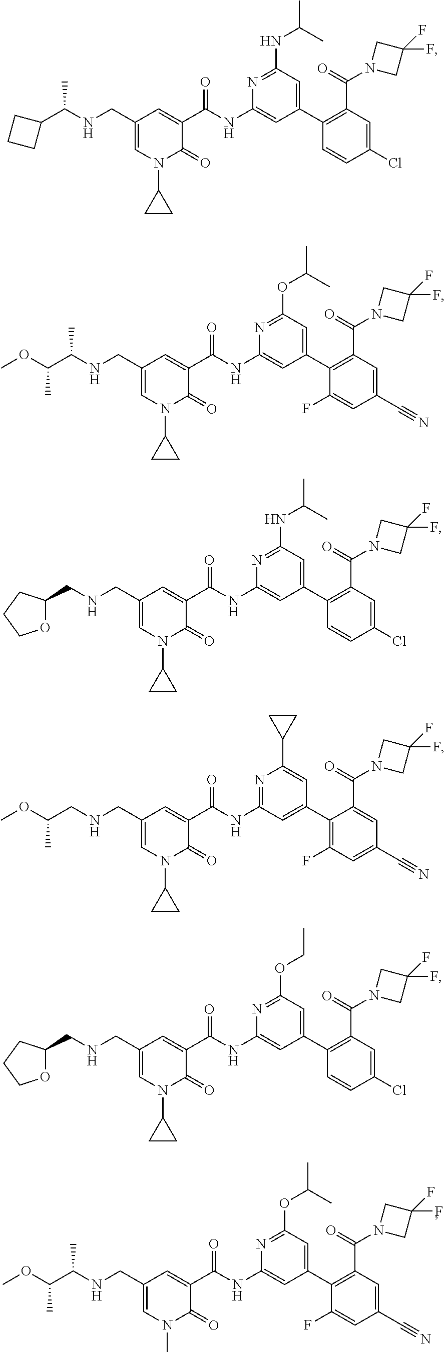

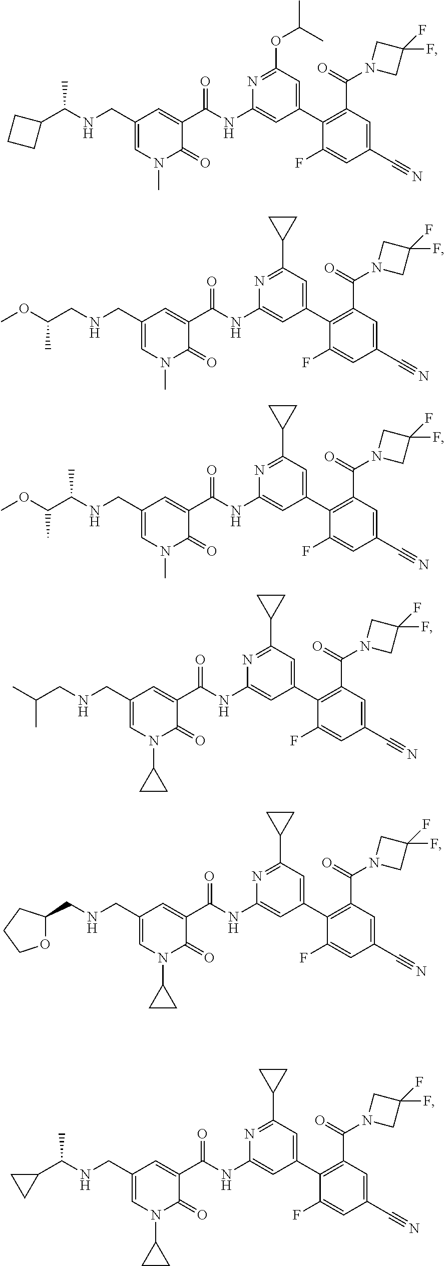

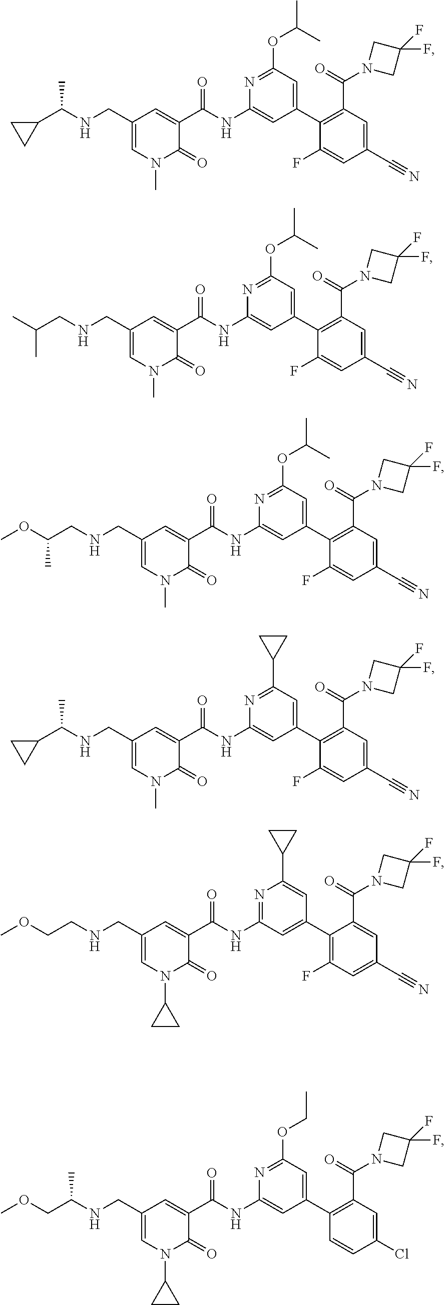

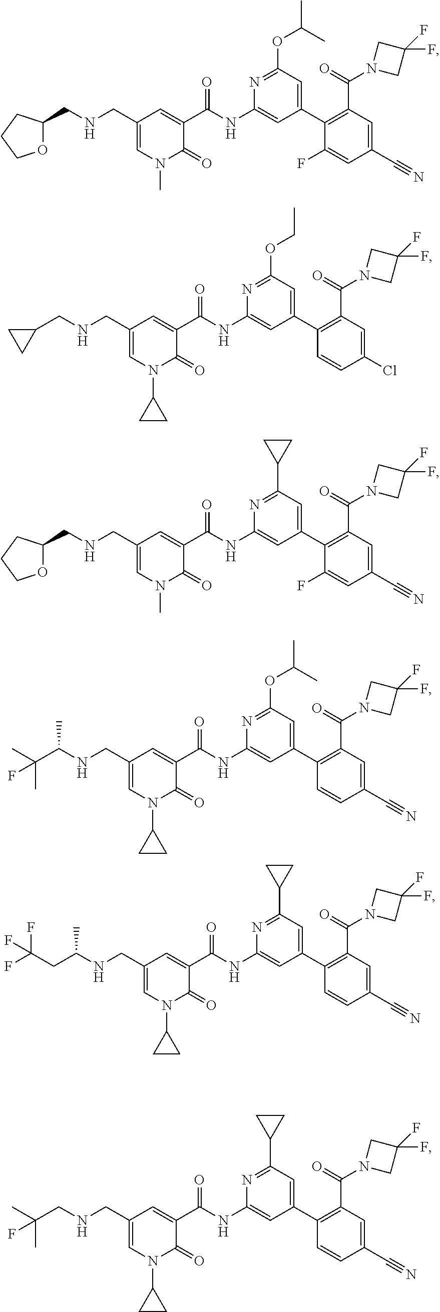

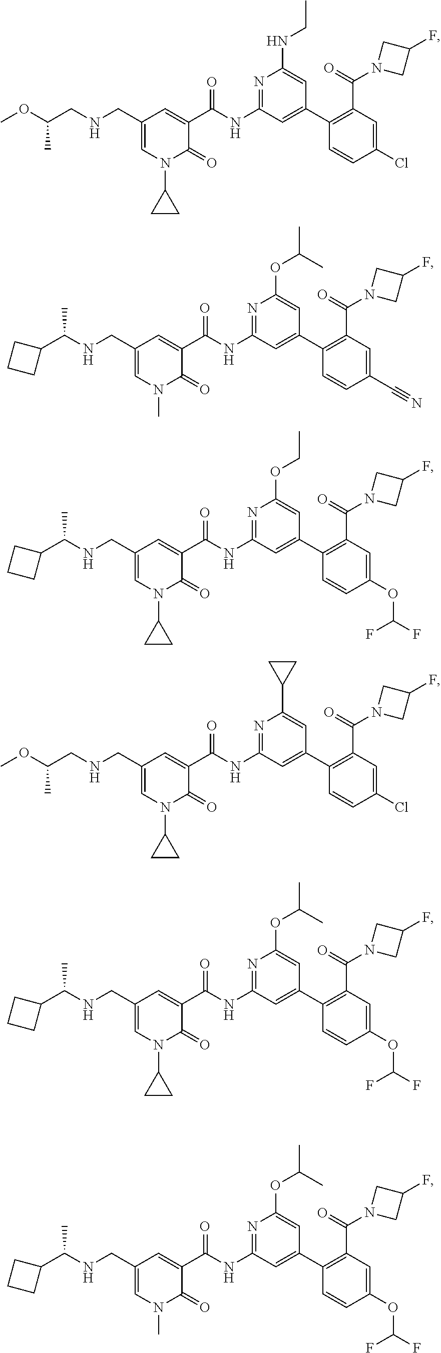

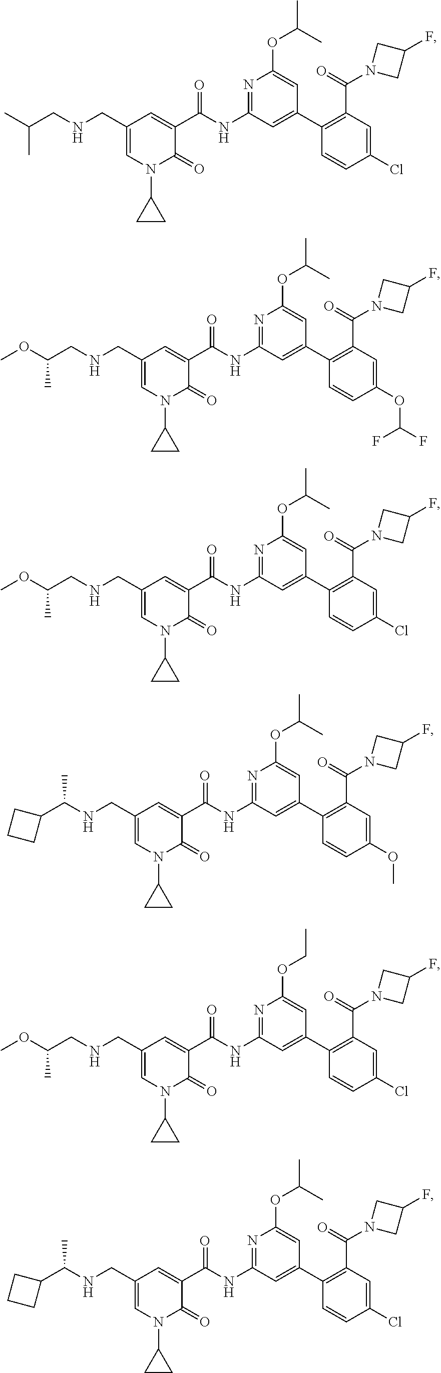

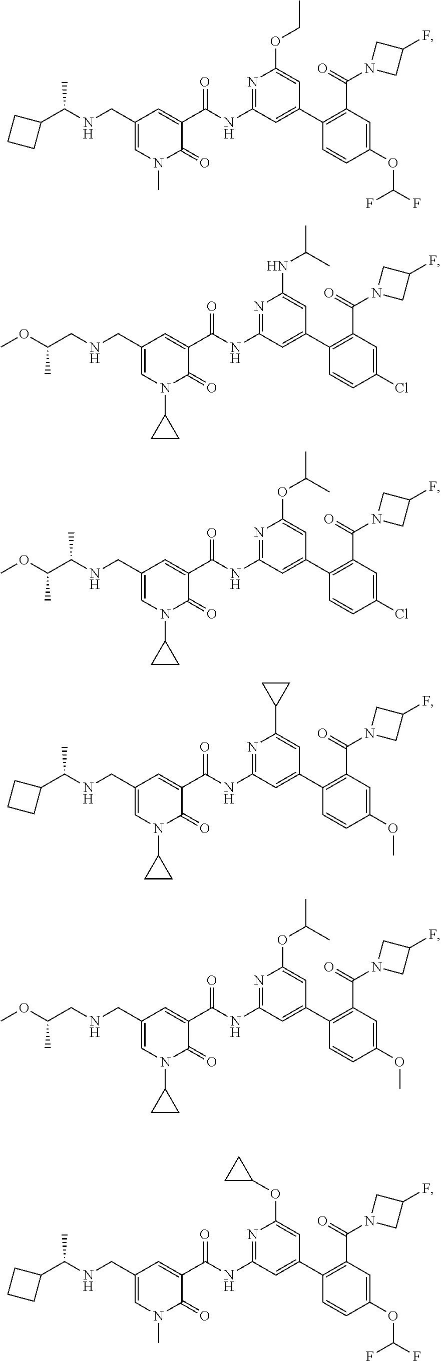

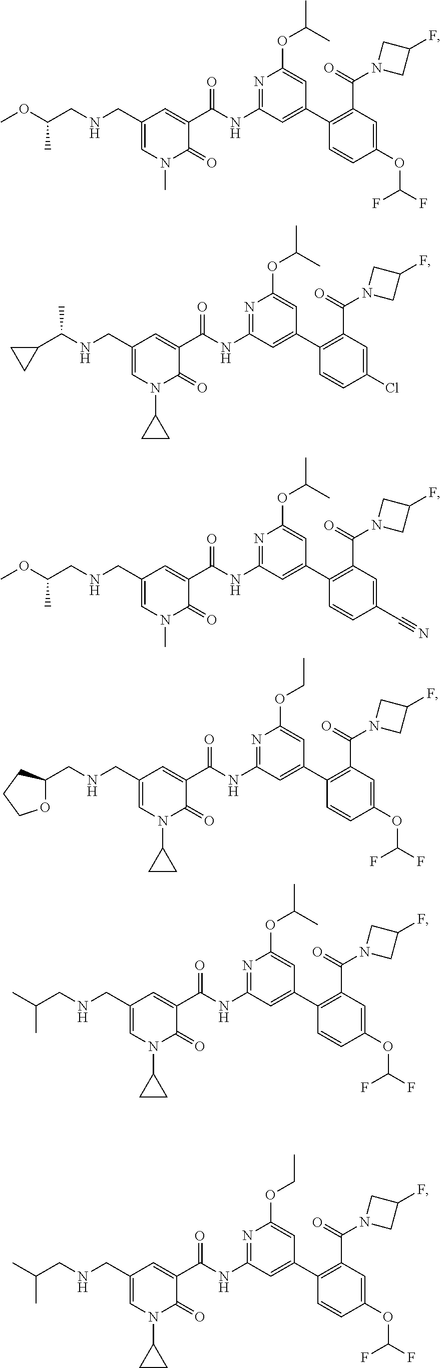

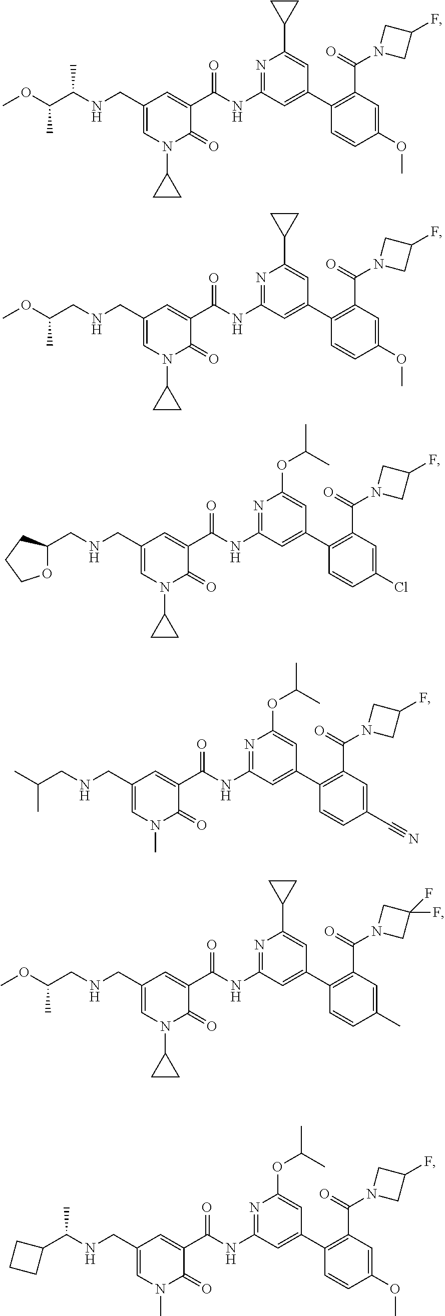

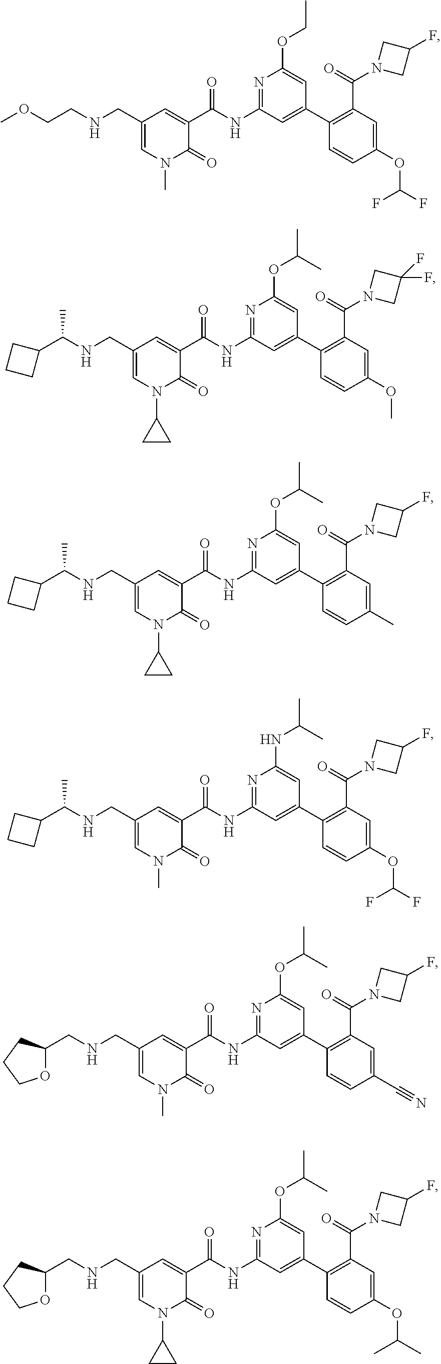

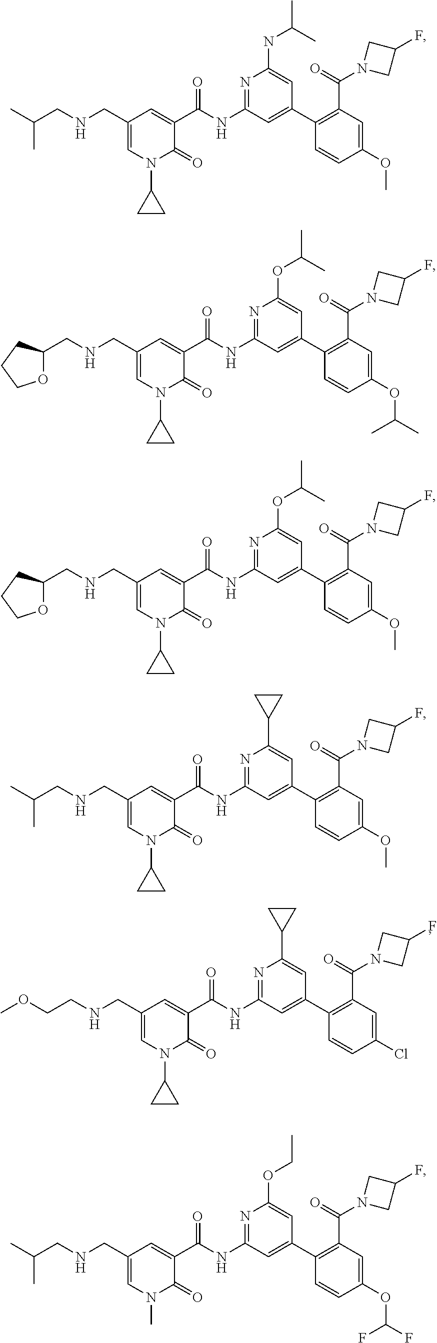

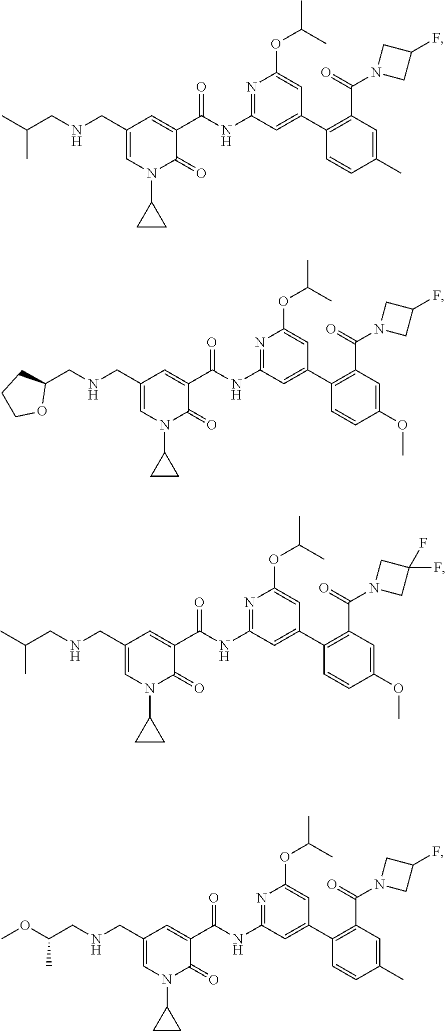 [SMILES: Cc1ccc(-c2cc(NC(=O)c3cc(CNCC(C)C)cn(C4CC4)c3=O)nc(OC(C)C)c2)c(C(=O)N2CC(F)C2)c1 ; COc1ccc(-c2cc(NC(=O)c3cc(CNC[C@@H]4CCCO4)cn(C4CC4)c3=O)nc(OC(C)C)c2)c(C(=O)N2CC(F)C2)c1 ; COc1ccc(-c2cc(NC(=O)c3cc(CNCC(C)C)cn(C4CC4)c3=O)nc(OC(C)C)c2)c(C(=O)N2CC(F)(F)C2)c1 ; CO[C@@H](C)CNCc1cc(C(=O)Nc2cc(-c3ccc(C)cc3C(=O)N3CC(F)C3)cc(OC(C)C)n2)c(=O)n(C2CC2)c1]

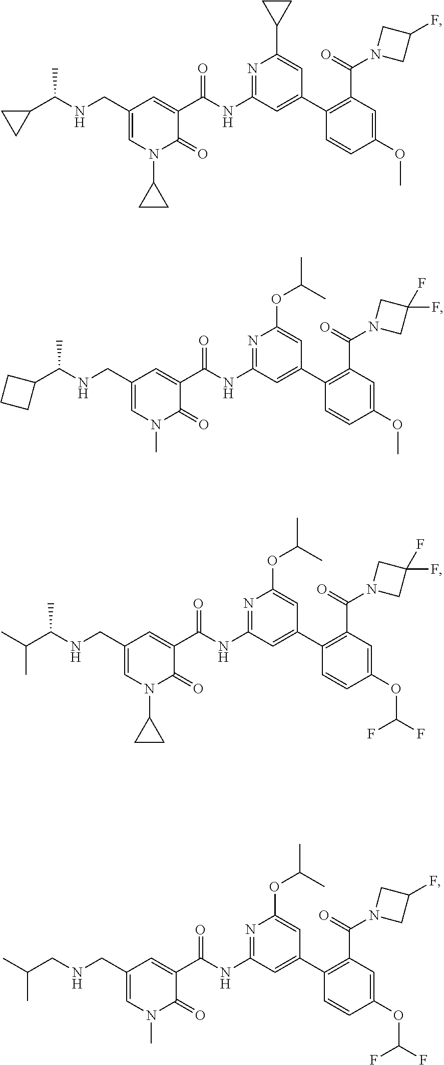 [SMILES: COc1ccc(-c2cc(NC(=O)c3cc(CN[C@@H](C)C4CC4)cn(C4CC4)c3=O)nc(C3CC3)c2)c(C(=O)N2CC(F)C2)c1 ; COc1ccc(-c2cc(NC(=O)c3cc(CN[C@@H](C)C4CCC4)cn(C)c3=O)nc(OC(C)C)c2)c(C(=O)N2CC(F)(F)C2)c1 ; CC(C)Oc1cc(-c2ccc(OC(F)F)cc2C(=O)N2CC(F)(F)C2)cc(NC(=O)c2cc(CN[C@@H](C)C(C)C)cn(C3CC3)c2=O)n1 ; CC(C)CNCc1cc(C(=O)Nc2cc(-c3ccc(OC(F)F)cc3C(=O)N3CC(F)C3)cc(OC(C)C)n2)c(=O)n(C)c1]

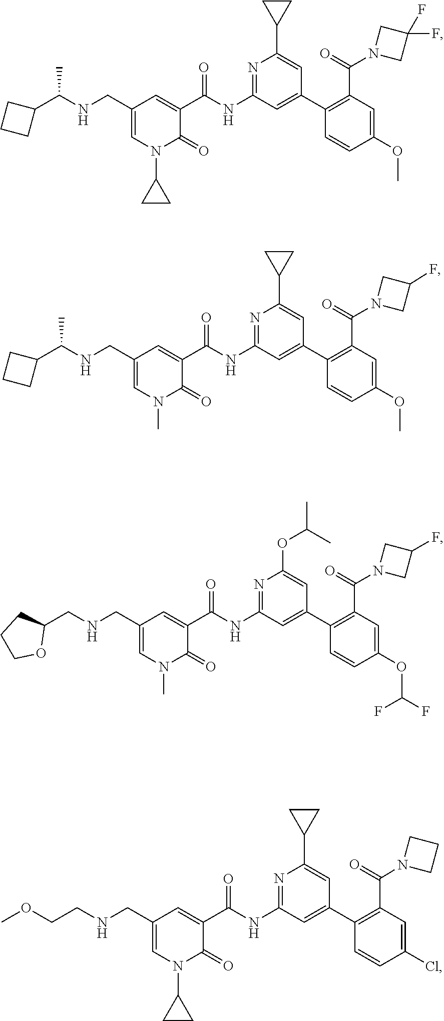 [SMILES: COc1ccc(-c2cc(NC(=O)c3cc(CN[C@@H](C)C4CCC4)cn(C4CC4)c3=O)nc(C3CC3)c2)c(C(=O)N2CC(F)(F)C2)c1 ; COc1ccc(-c2cc(NC(=O)c3cc(CN[C@@H](C)C4CCC4)cn(C)c3=O)nc(C3CC3)c2)c(C(=O)N2CC(F)C2)c1 ; CC(C)Oc1cc(-c2ccc(OC(F)F)cc2C(=O)N2CC(F)C2)cc(NC(=O)c2cc(CNC[C@@H]3CCCO3)cn(C)c2=O)n1 ; COCCNCc1cc(C(=O)Nc2cc(-c3ccc(Cl)cc3C(=O)N3CCC3)cc(C3CC3)n2)c(=O)n(C2CC2)c1]

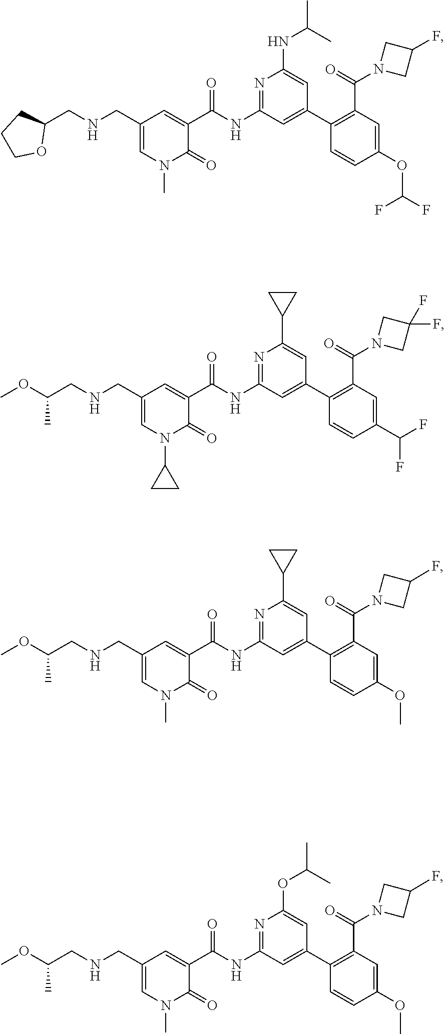 [SMILES: CC(C)Nc1cc(-c2ccc(OC(F)F)cc2C(=O)N2CC(F)C2)cc(NC(=O)c2cc(CNC[C@@H]3CCCO3)cn(C)c2=O)n1 ; CO[C@@H](C)CNCc1cc(C(=O)Nc2cc(-c3ccc(C(F)F)cc3C(=O)N3CC(F)(F)C3)cc(C3CC3)n2)c(=O)n(C2CC2)c1 ; COc1ccc(-c2cc(NC(=O)c3cc(CNC[C@H](C)OC)cn(C)c3=O)nc(C3CC3)c2)c(C(=O)N2CC(F)C2)c1 ; COc1ccc(-c2cc(NC(=O)c3cc(CNC[C@H](C)OC)cn(C)c3=O)nc(OC(C)C)c2)c(C(=O)N2CC(F)C2)c1]

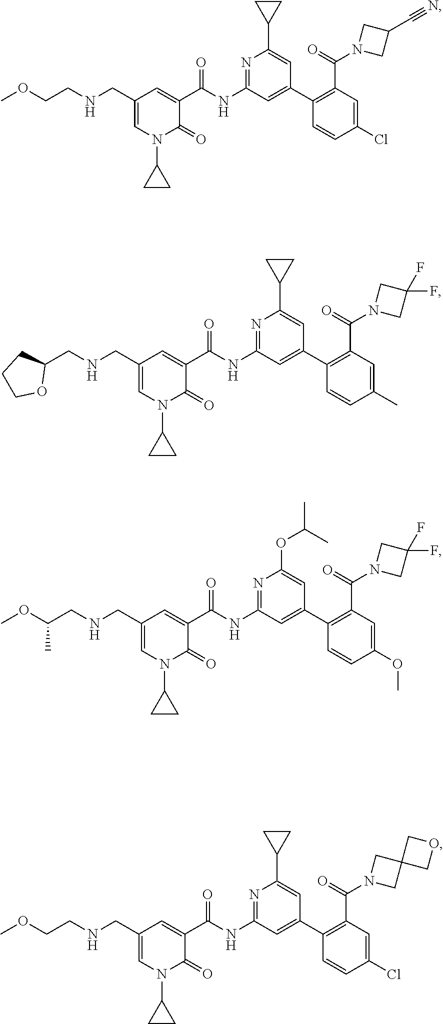 [SMILES: COCCNCc1cc(C(=O)Nc2cc(-c3ccc(Cl)cc3C(=O)N3CC(C#N)C3)cc(C3CC3)n2)c(=O)n(C2CC2)c1 ; Cc1ccc(-c2cc(NC(=O)c3cc(CNC[C@@H]4CCCO4)cn(C4CC4)c3=O)nc(C3CC3)c2)c(C(=O)N2CC(F)(F)C2)c1 ; COc1ccc(-c2cc(NC(=O)c3cc(CNC[C@H](C)OC)cn(C4CC4)c3=O)nc(OC(C)C)c2)c(C(=O)N2CC(F)(F)C2)c1 ; COCCNCc1cc(C(=O)Nc2cc(-c3ccc(Cl)cc3C(=O)N3CC4(COC4)C3)cc(C3CC3)n2)c(=O)n(C2CC2)c1]

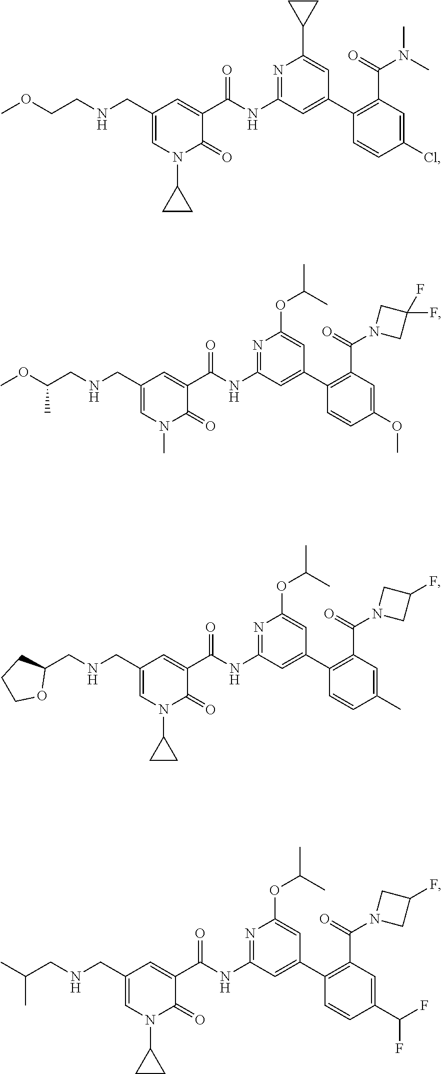

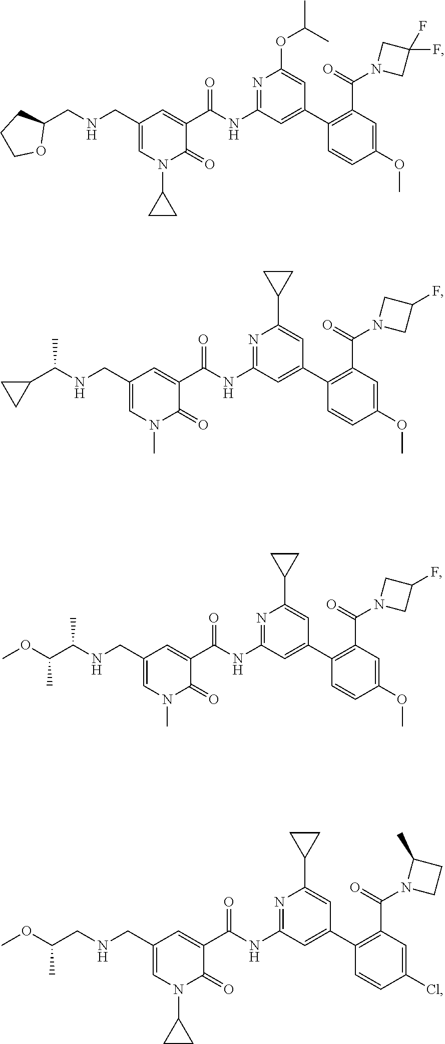 [SMILES: COc1ccc(-c2cc(NC(=O)c3cc(CNC[C@@H]4CCCO4)cn(C4CC4)c3=O)nc(OC(C)C)c2)c(C(=O)N2CC(F)(F)C2)c1 ; COc1ccc(-c2cc(NC(=O)c3cc(CN[C@@H](C)C4CC4)cn(C)c3=O)nc(C3CC3)c2)c(C(=O)N2CC(F)C2)c1 ; COc1ccc(-c2cc(NC(=O)c3cc(CN[C@@H](C)[C@H](C)OC)cn(C)c3=O)nc(C3CC3)c2)c(C(=O)N2CC(F)C2)c1 ; CO[C@@H](C)CNCc1cc(C(=O)Nc2cc(-c3ccc(Cl)cc3C(=O)N3CC[C@@H]3C)cc(C3CC3)n2)c(=O)n(C2CC2)c1]

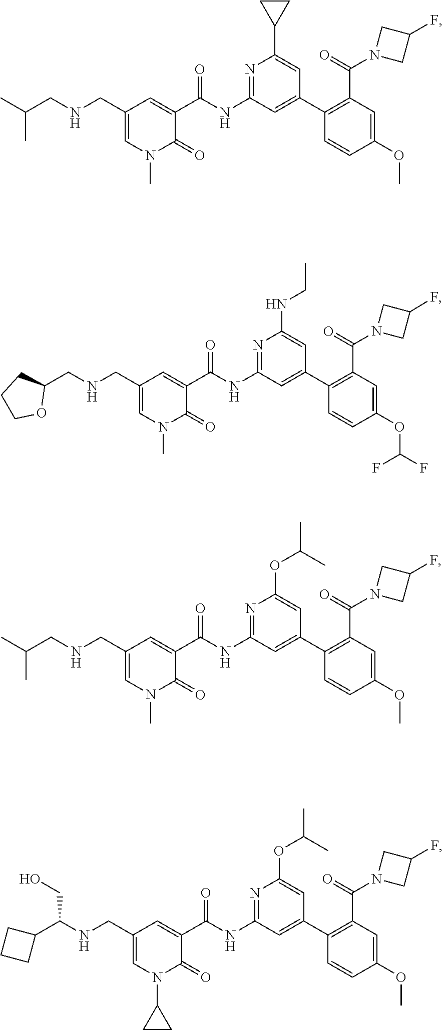 [SMILES: COc1ccc(-c2cc(NC(=O)c3cc(CNCC(C)C)cn(C)c3=O)nc(C3CC3)c2)c(C(=O)N2CC(F)C2)c1 ; CCNc1cc(-c2ccc(OC(F)F)cc2C(=O)N2CC(F)C2)cc(NC(=O)c2cc(CNC[C@@H]3CCCO3)cn(C)c2=O)n1 ; COc1ccc(-c2cc(NC(=O)c3cc(CNCC(C)C)cn(C)c3=O)nc(OC(C)C)c2)c(C(=O)N2CC(F)C2)c1 ; COc1ccc(-c2cc(NC(=O)c3cc(CN[C@@H](CO)C4CCC4)cn(C4CC4)c3=O)nc(OC(C)C)c2)c(C(=O)N2CC(F)C2)c1]

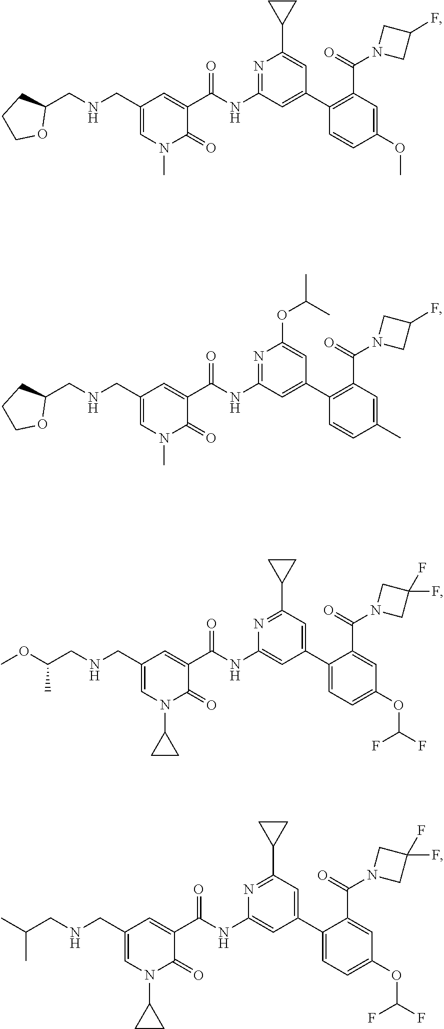 [SMILES: COc1ccc(-c2cc(NC(=O)c3cc(CNC[C@@H]4CCCO4)cn(C)c3=O)nc(C3CC3)c2)c(C(=O)N2CC(F)C2)c1 ; Cc1ccc(-c2cc(NC(=O)c3cc(CNC[C@@H]4CCCO4)cn(C)c3=O)nc(OC(C)C)c2)c(C(=O)N2CC(F)C2)c1 ; CO[C@@H](C)CNCc1cc(C(=O)Nc2cc(-c3ccc(OC(F)F)cc3C(=O)N3CC(F)(F)C3)cc(C3CC3)n2)c(=O)n(C2CC2)c1 ; CC(C)CNCc1cc(C(=O)Nc2cc(-c3ccc(OC(F)F)cc3C(=O)N3CC(F)(F)C3)cc(C3CC3)n2)c(=O)n(C2CC2)c1]

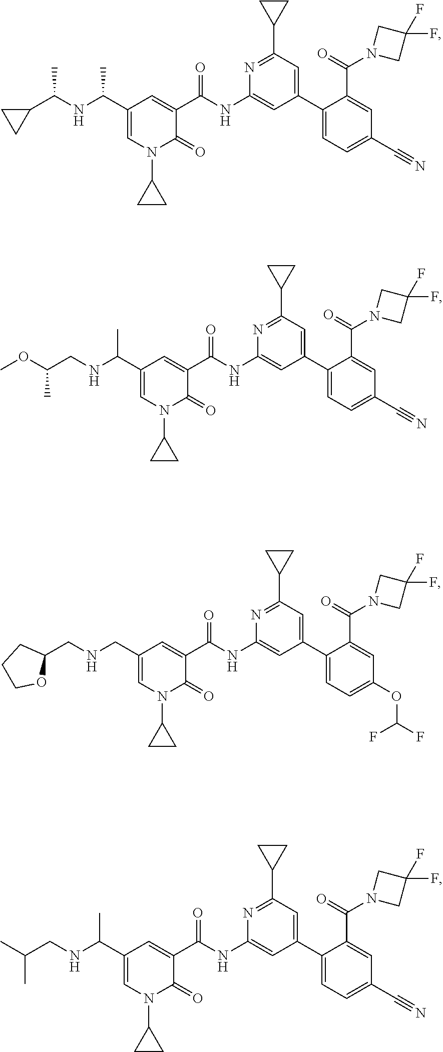 [SMILES: C[C@H](N[C@H](C)c1cc(C(=O)Nc2cc(-c3ccc(C#N)cc3C(=O)N3CC(F)(F)C3)cc(C3CC3)n2)c(=O)n(C2CC2)c1)C1CC1 ; CO[C@@H](C)CNC(C)c1cc(C(=O)Nc2cc(-c3ccc(C#N)cc3C(=O)N3CC(F)(F)C3)cc(C3CC3)n2)c(=O)n(C2CC2)c1 ; O=C(Nc1cc(-c2ccc(OC(F)F)cc2C(=O)N2CC(F)(F)C2)cc(C2CC2)n1)c1cc(CNC[C@@H]2CCCO2)cn(C2CC2)c1=O ; CC(C)CNC(C)c1cc(C(=O)Nc2cc(-c3ccc(C#N)cc3C(=O)N3CC(F)(F)C3)cc(C3CC3)n2)c(=O)n(C2CC2)c1]

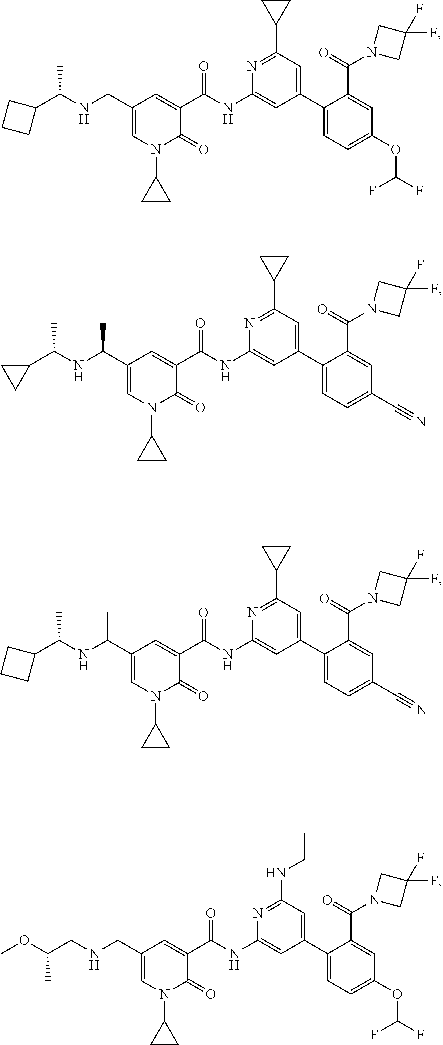 [SMILES: C[C@H](NCc1cc(C(=O)Nc2cc(-c3ccc(OC(F)F)cc3C(=O)N3CC(F)(F)C3)cc(C3CC3)n2)c(=O)n(C2CC2)c1)C1CCC1 ; C[C@H](N[C@@H](C)C1CC1)c1cc(C(=O)Nc2cc(-c3ccc(C#N)cc3C(=O)N3CC(F)(F)C3)cc(C3CC3)n2)c(=O)n(C2CC2)c1 ; CC(N[C@@H](C)C1CCC1)c1cc(C(=O)Nc2cc(-c3ccc(C#N)cc3C(=O)N3CC(F)(F)C3)cc(C3CC3)n2)c(=O)n(C2CC2)c1 ; CCNc1cc(-c2ccc(OC(F)F)cc2C(=O)N2CC(F)(F)C2)cc(NC(=O)c2cc(CNC[C@H](C)OC)cn(C3CC3)c2=O)n1]

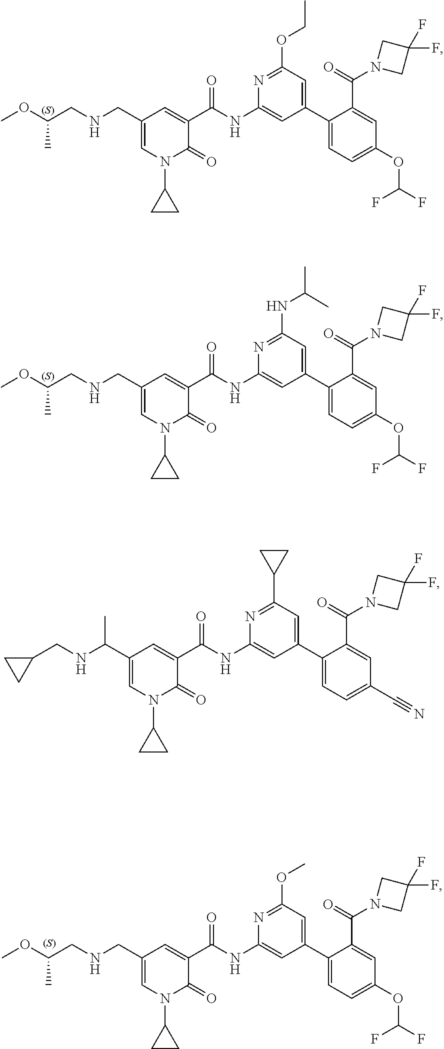

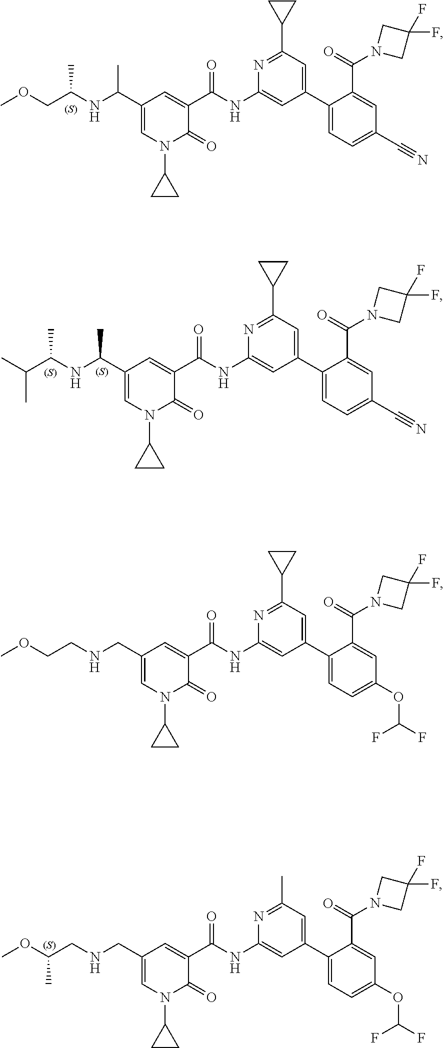

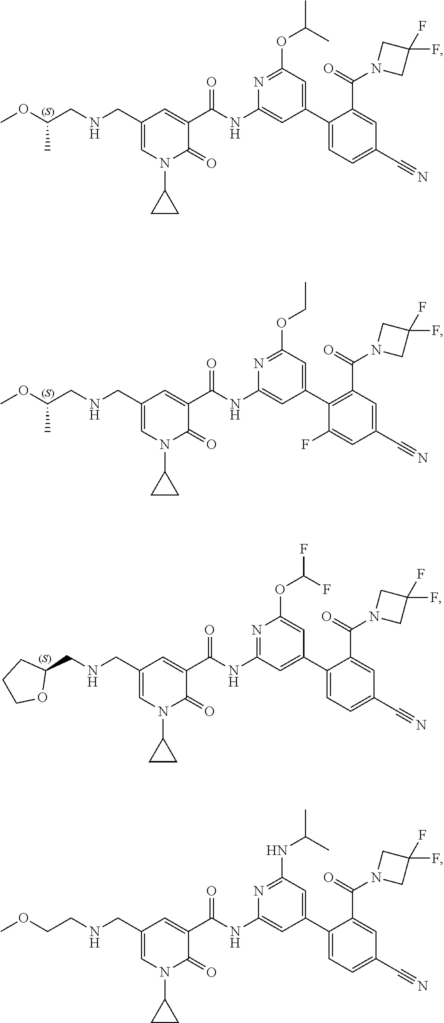

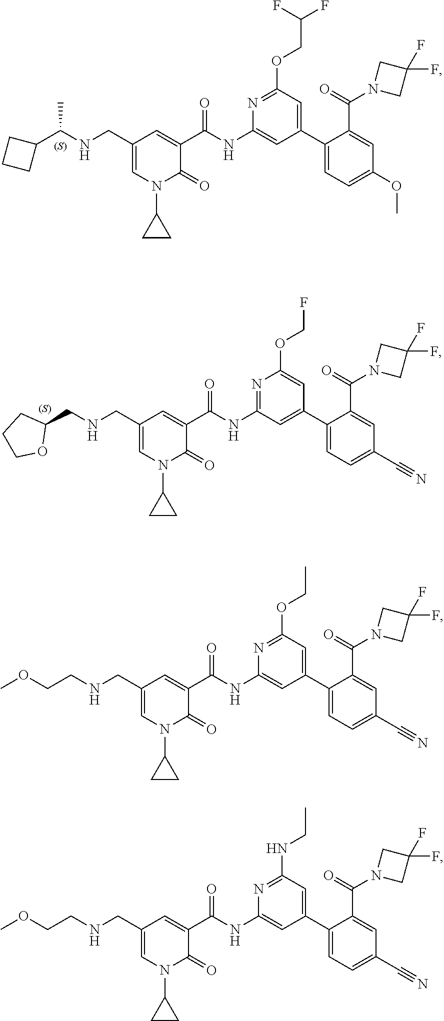

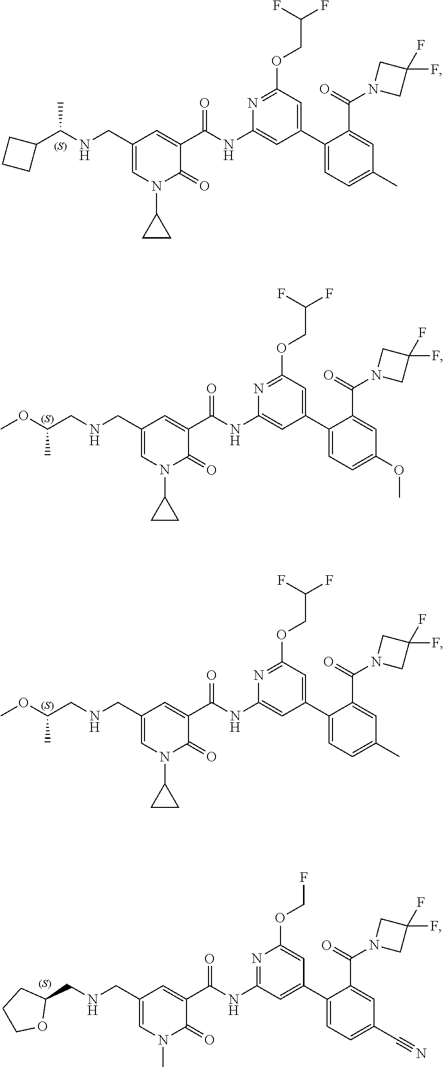

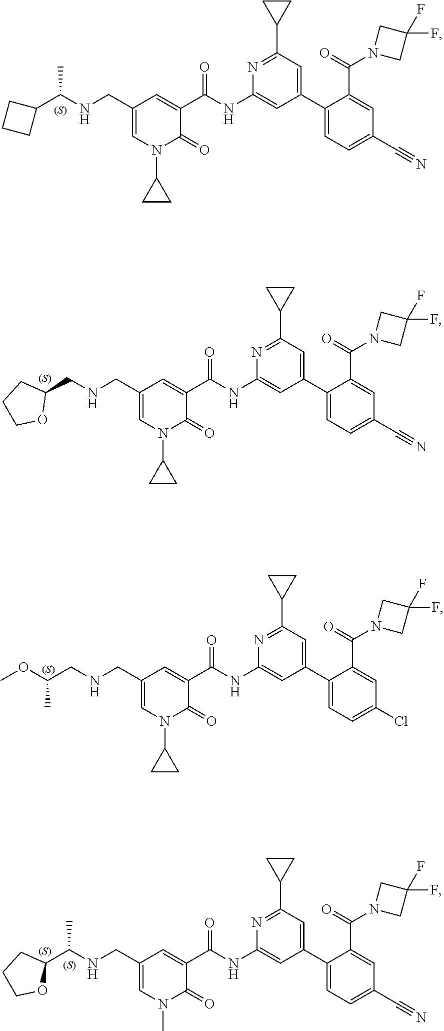

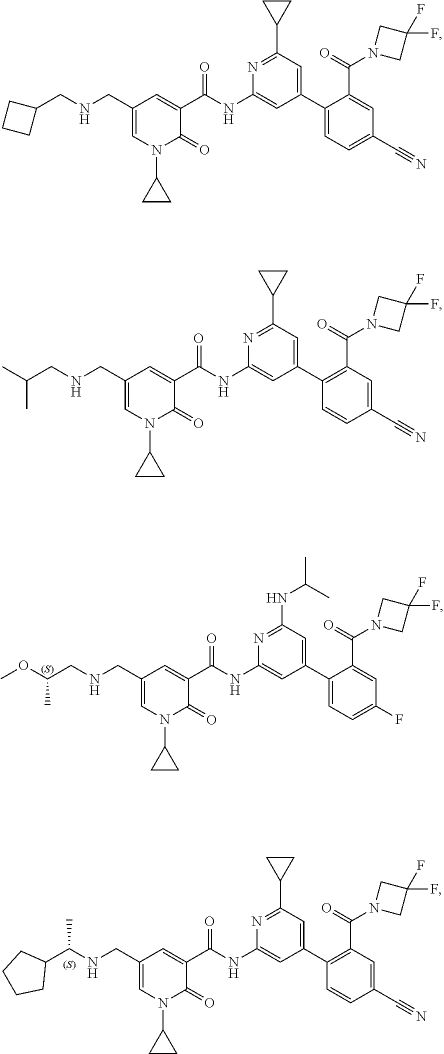

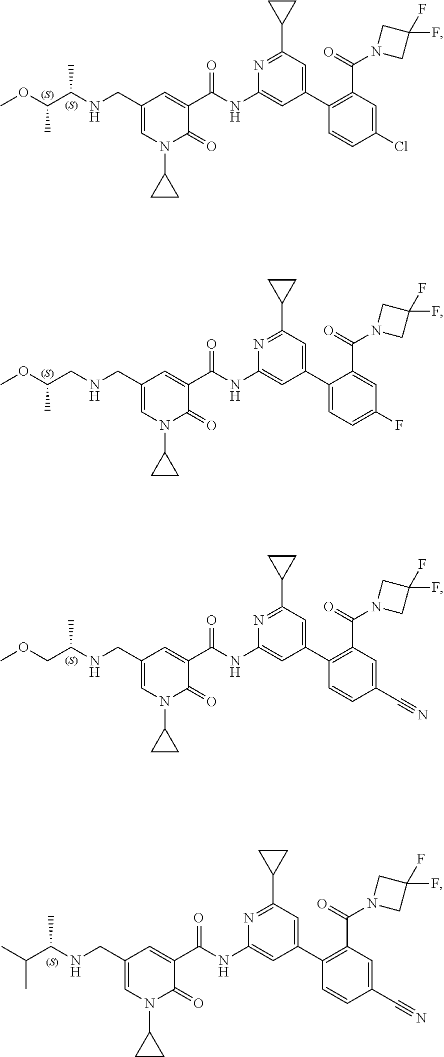

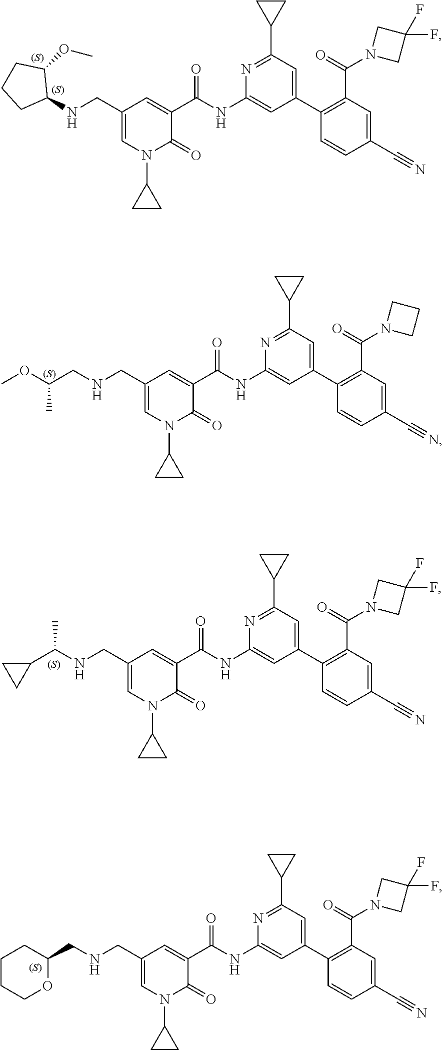

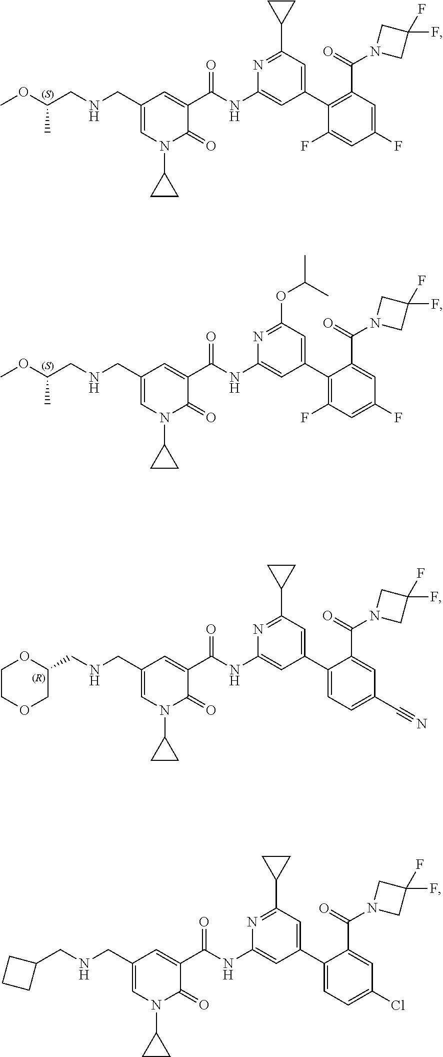

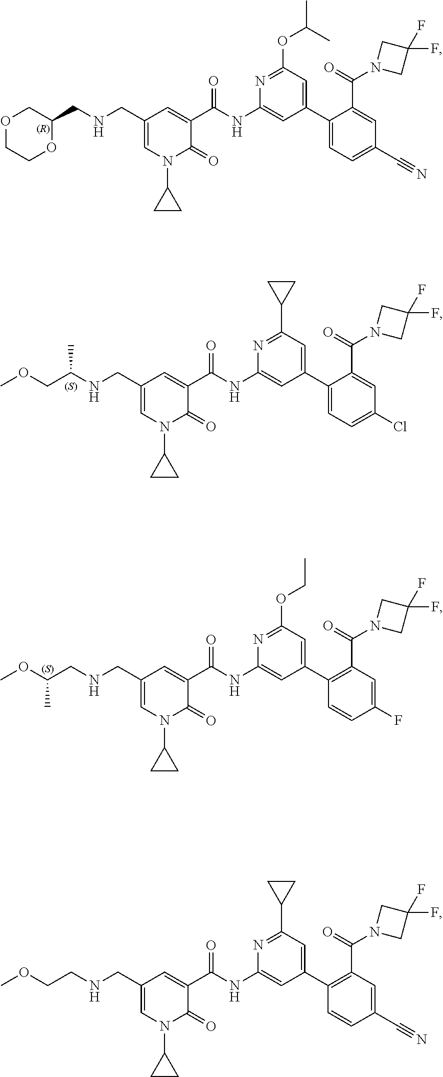

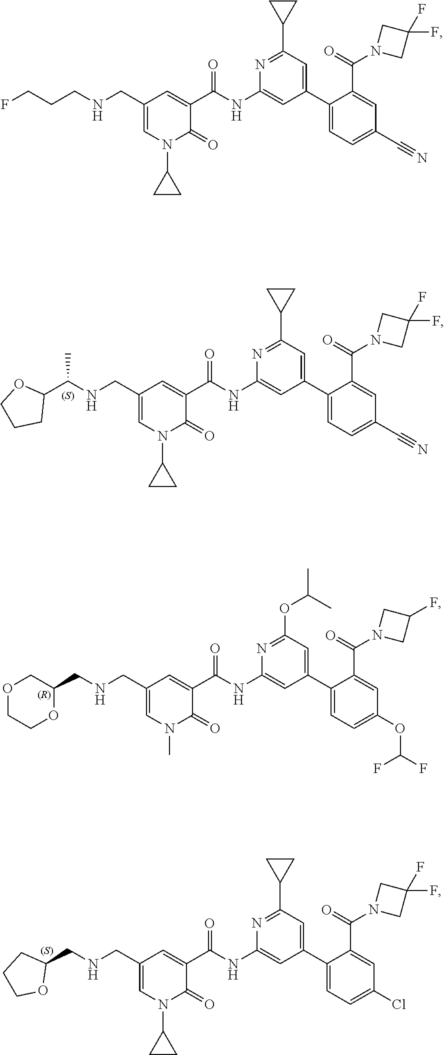

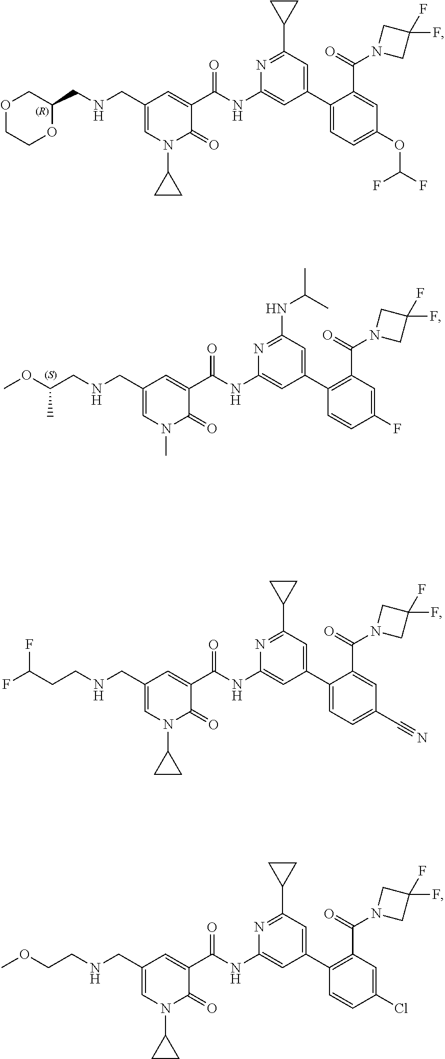

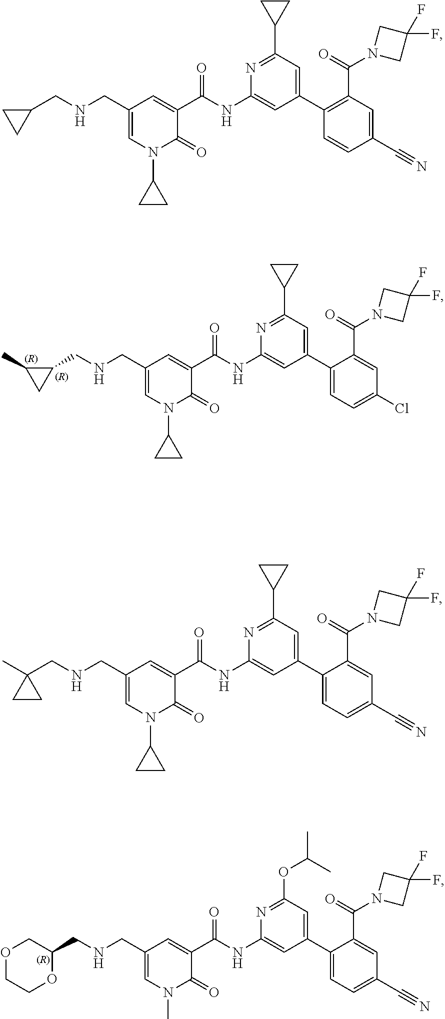

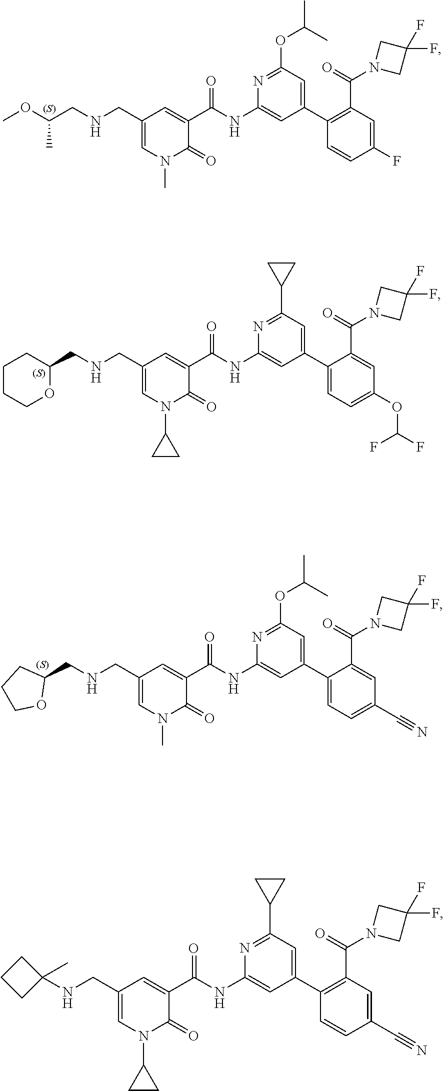

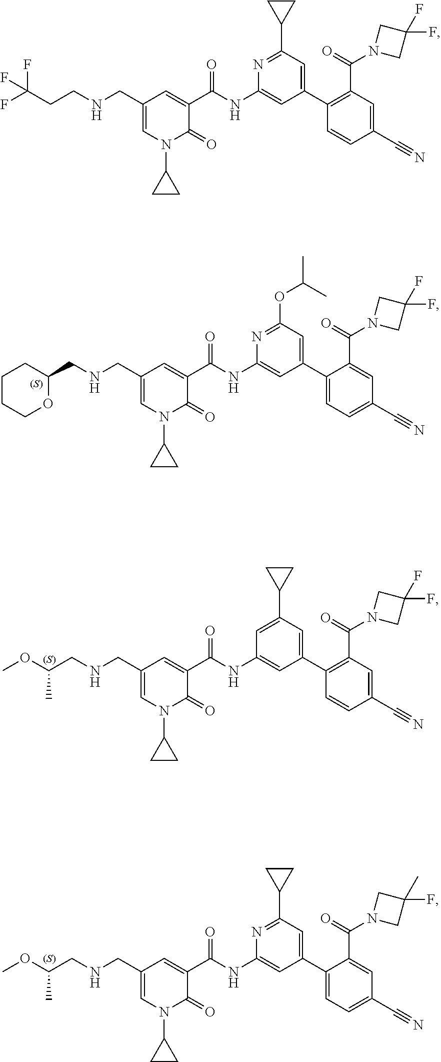

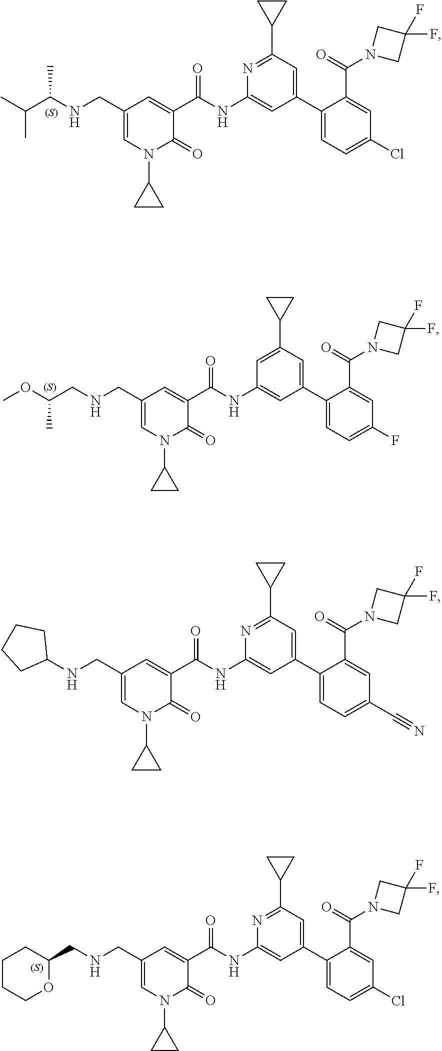

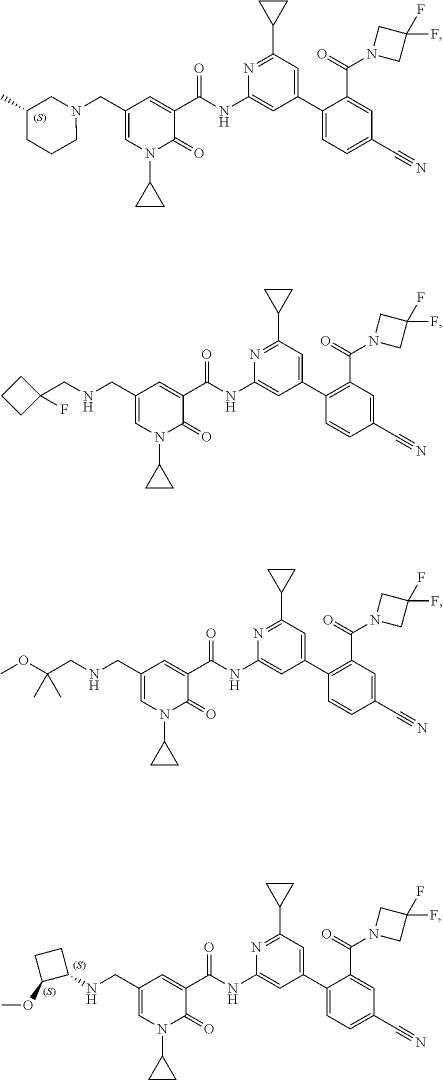

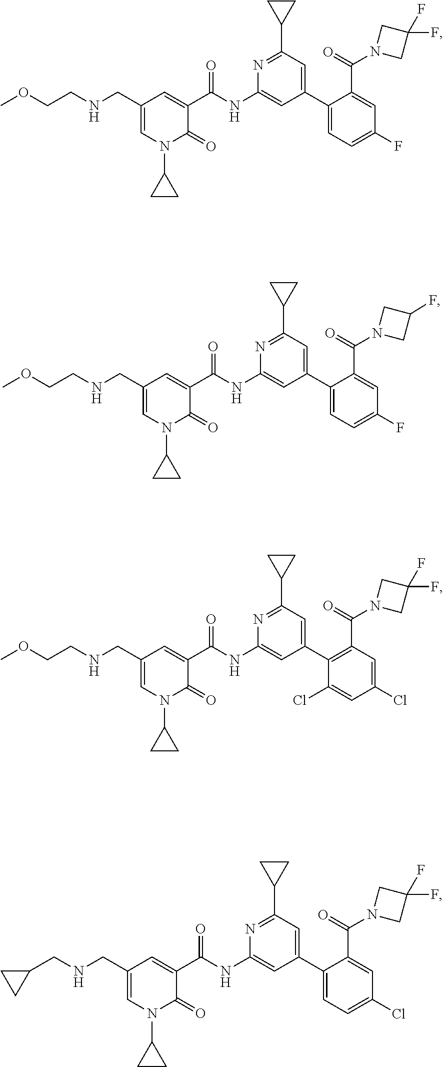 [SMILES: COCCNCc1cc(C(=O)Nc2cc(-c3ccc(F)cc3C(=O)N3CC(F)(F)C3)cc(C3CC3)n2)c(=O)n(C2CC2)c1 ; COCCNCc1cc(C(=O)Nc2cc(-c3ccc(F)cc3C(=O)N3CC(F)C3)cc(C3CC3)n2)c(=O)n(C2CC2)c1 ; COCCNCc1cc(C(=O)Nc2cc(-c3c(Cl)cc(Cl)cc3C(=O)N3CC(F)(F)C3)cc(C3CC3)n2)c(=O)n(C2CC2)c1 ; O=C(Nc1cc(-c2ccc(Cl)cc2C(=O)N2CC(F)(F)C2)cc(C2CC2)n1)c1cc(CNCC2CC2)cn(C2CC2)c1=O]

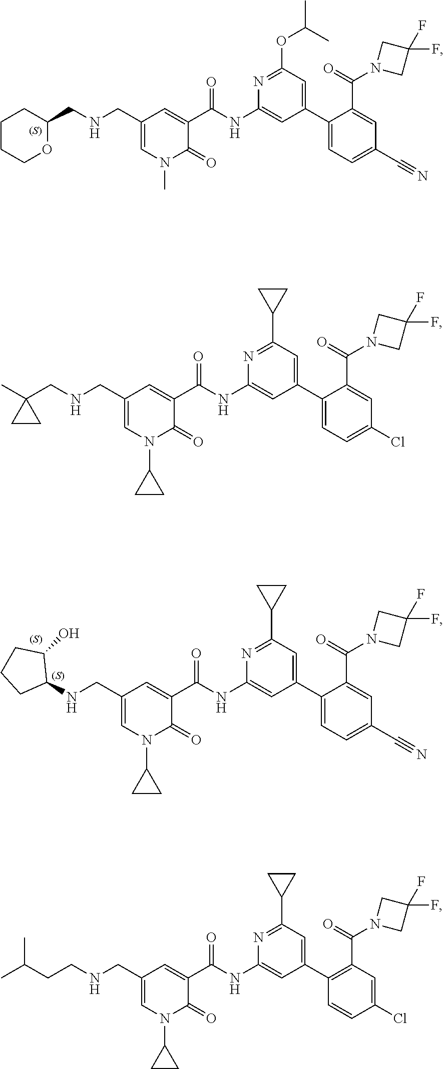

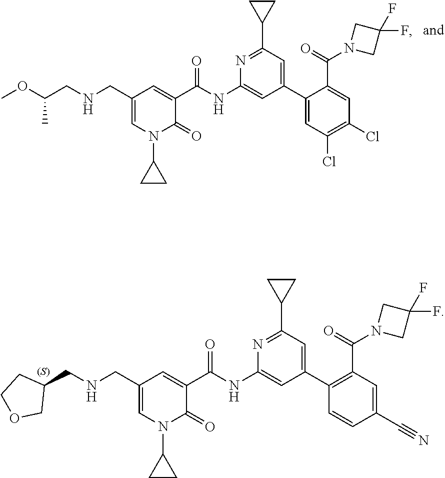

43. A pharmaceutical composition comprising a compound according to claim 1, or a pharmaceutically acceptable salt thereof, and a pharmaceutically acceptable excipient.

44-45. (canceled)

46. A method of treating a disease, disorder, or condition mediated at least in part by Cbl-b in a subject in need thereof, the method comprising administering to the subject a therapeutically effective amount of the compound of claim 1, or pharmaceutically acceptable salt thereof.

47. (canceled)

48. The method of claim 46, wherein the disease, disorder, or condition is cancer.

49-51. (canceled)

52. The method of claim 48, further comprising administering at least one additional therapeutic agent to the subject.

53. The method of claim 52, wherein said at least one additional therapeutic agent comprises one or more agents independently selected from the groups consisting of immune checkpoint inhibitors, agents that target the extracellular production of adenosine, inhibitors of HIF, kinase inhibitors, radiation therapy, and chemotherapeutic agents.
